(19) United States
(12) Patent Application Publication
(10) Pub. No.: US 2015/0344457 A1
(43) Pub. Date: Dec. 3, 2015

(54) METHODS OF INHIBITING PRMT5

(71) Applicant: EPIZYME, INC., Cambridge, MA (US)

(72) Inventors: Kenneth W. Duncan, Westwood, MA (US); Richard Chesworth, Concord, MA (US); Paula Ann Boriack-Sjodin, Lexington, MA (US); Lei Jin, Wellesley, MA (US)

(73) Assignee: Epizyme, inc., Cambridge, MA (US)

(21) Appl. No.: 14/654,213

(22) PCT Filed: Dec. 20, 2013
(86) PCT No.: PCT/US2013/077308

§ 371 Date: Jun. 19, 2015

Related U.S. Application Data
(60) Provisional application No. 61/745,537, filed on Dec. 21, 2012.

(51) Int. Cl. C07D 401/12; C07D 405/12; C07D 401/14; C07D 413/12; C07D 405/14; G06F 17/50; C07D 487/04; C07D 409/12; C07D 471/04; G01N 33/573; G06F 19/16; C07D 217/04; C07D 413/14

(52) U.S. Cl. CPC C07D 401/12; C07D 217/04; C07D 405/12; C07D 401/14; C07D 413/12; C07D 405/14; C07D 413/14; C07D 487/04; C07D 409/12; C07D 471/04; G01N 33/573; G06F 19/16; G06F 17/50; G01N 2333/91011

(57) ABSTRACT

Described herein are compounds of Formula (I) useful for inhibiting PRMT5 activity. The planes of Ring AA and Ring BB are between 75° and 105°. Ring AA-M-Ring BB (I)

### RELATED APPLICATIONS

The present application claims priority under 35 U.S.C. §119(e) to U.S. provisional patent application, U.S. Ser. No. 61/745,537, filed Dec. 21, 2012, the entire contents of which is incorporated herein by reference.

### BACKGROUND OF THE INVENTION

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., PRMT5), many of which are associated with specific genetic alterations that can cause human disease.

Disease-associated chromatin-modifying enzymes (e.g., PRMT5) play a role in diseases such as proliferative disorders, metabolic disorders, and blood disorders. Thus, there is a need for the development of small molecules that are capable of modulating the activity of PRMT5.

### BRIEF DESCRIPTION OF THE FIGURES

The drawings are exemplary and not required for enablement of the invention.

FIG. 1 shows examples of PRMT5-MEP50-compound crystals.

FIG. 2 depicts a stick-and-ribbon representation of the active site of PRMT5-Compound-FIG. 2.

FIG. 3 depicts a stick-and-ribbon representation of the active site of PRMT5-Compound A6.

FIG. 4 depicts a stick-and-ribbon representation of the active site of PRMT5-Compound-FIG. 4.

FIG. 5 depicts a stick-and-ribbon representation of the active site of PRMT5-Compound-FIG. 5.

FIG. 6 depicts a stick-and-ribbon representation of the active site of PRMT5-Compound-FIG. 6.

### DESCRIPTION

PRMT5 is an attractive target for modulation given its role in the regulation of diverse biological processes. It has now been found that compounds described herein, and pharmaceutically acceptable salts and compositions thereof, are effective as inhibitors of PRMT5 Inhibitors of PRMT5 may be useful in the treatment of a wide variety of diseases including proliferative disease (e.g., cancer), inflammatory diseases, autoimmune diseases, metabolic diseases (e.g., diabetes, obesity), and hematological diseases (e.g., hemoglobinopathies such as sickle cell disease).

In one aspect, the compounds described herein have Formula (I):

Ring AA-M-Ring BB I

wherein Ring AA, M and Ring BB are as defined herein. In certain embodiments, Ring AA is an optionally substituted aryl moiety; Ring BB is an optionally substituted aryl or heteroaryl moiety; M is an acyclic linker moiety 3-10 atoms in length; the planes of Ring AA and Ring BB to be between 75° and 105° relative to each other. In certain embodiments, the compounds of Formula (I) inhibit PRMT5 with an IC₅₀ less than 100 nM.

In another aspect, such compounds have Formula (II):

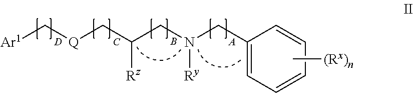

wherein Ar′, Q, Rx, Ry, Rz are as defined herein.

In another aspect, the disclosure provides compounds that inhibit PRMT5. The disclosure provides the structural parameters of a class of compounds that inhibit PRMT5. The disclosure provides compounds with structural elements for binding in the active site of PRMT5, thereby inhibiting the function (e.g., enzymatic activity) of PRMT5. The disclosure also provides structural elements of a compound that interact with S-adenosyl methionine (SAM) in the active site of PRMT5, thereby inhibiting the function of PRMT5. For example, in certain embodiments, the compounds disclosed herein possess an aryl moiety that interacts with SAM through a pi-cation interaction. In some embodiments, the compounds disclosed herein possess an aryl moiety that interacts with Phe327 of PRMT5 through a pi-stacking interaction.

In another aspect, the disclosure provides methods for designing and/or identifying compounds that bind PRMT5 comprising generating, on a computer, a three-dimensional structure of PRMT5 having the structural coordinates of Table A, followed by identifying amino acid residues forming the active site. The identified amino acids can be used to generate a three-dimensional model of the active site for further designing and/or selecting a compound that potentially binds to the active site. In certain embodiments, the active site of PRMT5 is modeled using S-adenosyl methionine (SAM), or an analog thereof, and amino acids Leu319, Phe327, Glu435, Leu437, Glu444, and Phe580.

In another aspect, the disclosure provides methods for designing and identifying compounds that bind protein arginine N-methyltransferase 5 (PRMT5) comprising the steps of: (a) generating, on a computer, a three-dimensional structure of methyltransferase PRMT5 having the structural coordinates of Table A; (b) identifying amino acid residues forming the active site of PRMT5 in three-dimensions from step (a), wherein the active site comprises S-adenosyl methionine (SAM), or an analog thereof, and amino acids Leu319, Phe327, Glu435, Leu437, Glu444, and Phe580, according to Table A; (c) generating a three-dimensional model of the active site; (d) designing and/or selecting a compound that potentially binds to the active site using the three-dimensional model of the active site; and (e) optionally, synthesizing and/or choosing the potential binding compound.

In another aspect, the disclosure provides methods of identifying a compound that binds protein arginine N-methyltransferase 5 (PRMT5), the method comprising computationally identifying a compound that binds to PRMT5 using the atomic coordinates of S-adenosyl methionine (SAM), or an analog thereof, and amino acids Leu319, Phe327, Glu435, Leu437, Glu444, and Phe580, according the atomic coordinates provided in Table A.

In another aspect, the disclosure provides methods of identifying a compound that binds protein arginine N-methyltransferase 5 (PRMT5), the method comprising computationally identifying a binding compound that binds to PRMT5 using the atomic coordinates of S-adenosyl methionine (SAM) and amino acids Leu319, Phe327, Lys333, Glu435, Leu437, Gly438, Ser439, Glu444, Val503, Ser578, Trp579, and Phe580, according to Table A.

In another aspect, the disclosure provides a method of identifying a binding compound of protein arginine N-methyltransferase 5 (PRMT5), the method comprising: computationally identifying a binding compound that binds to PRMT5 using the atomic coordinates of S-adenosyl methionine (SAM) and amino acids Leu312, Leu319, Thr323, Tyr324, Phe327, Glu328, Lys333, Tyr334, Glu435, Leu437, Gly438, Ser439, Glu444, Val503, Ser578, Trp579, Phe580, and Pro581, according to the atomic coordinates provided in Table A.

In one aspect, the disclosure provides compounds that inhibit PRMT5. In one aspect, the disclosure describes structural elements useful for a compound to bind in the active site of PRMT5. In one aspect, the disclosure provides what structural elements are needed for a compound of the formula Ring AA-M-Ring BB to act as an inhibitor of PRMT5. It was surprisingly found that compounds of the formula Ring AA-M-Ring BB, wherein M is a linker that allows for the planes of Ring AA and Ring BB to be at about 90° relative to each other, inhibit the enzymatic activity of PRMT5. Compounds of the formula Ring AA-M-Ring BB with the recited geometry fit into the PRMT5 active site, thereby inhibiting the enzymatic activity of PRMT5. In some embodiments, compounds of the formula Ring AA-M-Ring BB, ring BB can form a pi-cation interaction with one more amino acids in the active site. In some embodiments, compounds of the formula Ring AA-M-Ring BB, ring BB can form a pi-stacking interaction with one more amino acids in the active site. In some embodiments, compounds of the formula Ring AA-M-Ring BB with the recited geometry fit in the active site because they are capable of a pi-cation interaction with the PRMT5-bound S-adenosyl methionine (SAM) of the active site. In some embodiments, compounds of the formula Ring AA-M-Ring BB with the recited geometry fit in the active site because they are capable of undergoing a pi-stacking interaction with Phe327. In some embodiments, compounds of the formula Ring AA-M-Ring BB with the recited geometry fit in the active site because the flexibility of the linker allows Ring BB to interact with SAM and Phe327, while at the same time allowing Ring AA to interact with Ring BB.

In one aspect, the active site of PRMT5 comprises amino acids Leu319, Phe327, Glu435, Leu437, Glu444, and Phe580. In certain embodiments, the active site of PRMT5 comprises S-adenosyl methionine (SAM) and amino acids Leu319, Phe327, Glu435, Leu437, Glu444, and Phe580. In some embodiments, the active site of PRMT5 comprises S-adenosyl methionine (SAM) and amino acids Leu319, Phe327, Lys333, Glu435, Leu437, Gly438, Ser439, Glu444, Val503, Ser578, Trp579, and Phe580. In some embodiments, the active site of PRMT5 comprises S-adenosyl methionine (SAM) and amino acids Leu312, Leu319, Thr323, Tyr324, Phe327, Glu328, Lys333, Tyr334, Glu435, Leu437, Gly438, Ser439, Glu444, Val503, Ser578, Trp579, Phe580, and Pro581. In some embodiments, the atomic coordinates of the active site are provided in Table A. It should be appreciated that the active site may contain a SAM analog (e.g., sinefungin) instead of SAM.

In another aspect, the disclosure provides PRMT5 inhibitors having molecular dimensions compatible with the shape of the PRMT5-active site as defined by the atomic coordinates of amino acids Leu319, Phe327, Glu435, Leu437, Glu444, and Phe580 and, optionally, further including s-adenosyl methionine (SAM), according to Table A, wherein the compound has an IC₅₀ for PRMT5 of less than 100 nM.

In another aspect, the disclosure provides compositions comprising PRMT5 and a compound of the formula Ring AA-M-Ring BB. In some embodiments, the composition is an isolated composition comprising PRMT5 and a compound of the formula Ring AA-M-Ring BB. In some embodiments, the composition is a co-crystal comprising PRMT5 and a compound of the formula Ring AA-M-Ring BB.

In another aspect, the disclosure provides a computer readable medium comprising the atomic coordinates of the complex of PRMT5, and Compound A6 as set forth in Table A1

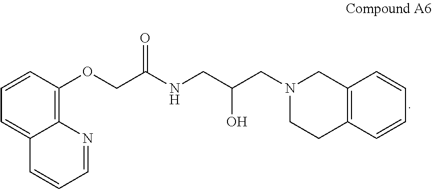 [SMILES: O=C(COc1cccc2cccnc12)NCC(O)CN1CCc2ccccc2C1]

In another aspect, the disclosure provides a crystal structure of the complex PRMT5-Compound A6.

In another aspect, the disclosure provides kits comprising any of the disclosed compounds, or a pharmaceutically acceptable salt thereof, or pharmaceutical compositions thereof, and instructions for use.

In another aspect, the disclosure provides methods of inhibiting PRMT5 comprising contacting a cell with an effective amount of a compound, or a pharmaceutically acceptable salt thereof, or composition thereof.

In another aspect, the disclosure provides methods of altering gene expression comprising contacting a cell with an effective amount of a compound disclosed herein, or a pharmaceutically acceptable salt thereof, or a composition thereof.

In another aspect, the disclosure provides methods of altering transcription in a call comprising contacting a cell with an effective amount of a compound disclosed herein, or a pharmaceutically acceptable salt thereof, or a composition thereof.

In another aspect, the disclosure provides methods of treating a PRMT5-mediated disorder comprising administering to a subject in need thereof a therapeutically effective amount of a compound disclosed herein, or a pharmaceutically acceptable salt thereof, or a pharmaceutical composition thereof.

In certain embodiments, compounds described herein inhibit activity of PRMT5 by interacting with the S-adenosyl methionine (SAM) in the active site of PRMT5. In some embodiments the compounds described herein are designed in silico. In some embodiments the compounds are designed based on the crystal structure coordinates provides herein (See e.g., Table A)

In certain embodiments, compounds described herein inhibit activity of PRMT5. In certain embodiments, methods of inhibiting PRMT5 are provided which comprise contacting PRMT5 with an effective amount of a compound of Formula (I) or (II), or a pharmaceutically acceptable salt thereof. The PRMT5 may be purified or crude, and may be present in a cell, tissue, or a subject. Thus, such methods encompass inhibition of PRMT5 activity in vitro and in vivo. In certain embodiments, the PRMT5 is wild-type PRMT5. In certain embodiments, the PRMT5 is overexpressed. In certain embodiments, the PRMT5 is a mutant. In certain embodiments, the PRMT5 is in a cell. In certain embodiments, the PRMT5 is in an animal, e.g., a human. In some embodiments, the PRMT5 is in a subject that is susceptible to normal levels of PRMT5 activity due to one or more mutations associated with a PRMT5 substrate. In some embodiments, the PRMT5 is in a subject known or identified as having abnormal PRMT5 activity (e.g., overexpression). In some embodiments, a provided compound is selective for PRMT5 over other methyltransferases. In certain embodiments, a provided compound is at least about 10-fold selective, at least about 20-fold selective, at least about 30-fold selective, at least about 40-fold selective, at least about 50-fold selective, at least about 60-fold selective, at least about 70-fold selective, at least about 80-fold selective, at least about 90-fold selective, or at least about 100-fold selective relative to one or more other methyltransferases.

In certain embodiments, methods of altering gene expression in a cell are provided which comprise contacting a cell with an effective amount of a compound of Formula (I), or a pharmaceutically acceptable salt thereof, or a composition thereof. In certain embodiments, the cell is cultured in vitro. In certain embodiments, the cell is in an animal, e.g., a human.

In certain embodiments, methods of altering transcription in a cell are provided which comprise contacting a cell with an effective amount of a compound of Formula (I), or a pharmaceutically acceptable salt thereof, or a composition thereof. In certain embodiments, the cell in culture in vitro. In certain embodiments, the cell is in an animal, e.g., a human.

In some embodiments, methods of treating a PRMT5-mediated disorder are provided which comprise administering to a subject suffering from a PRMT5-mediated disorder an effective amount of a compound described herein (e.g., a compound of Formula (I)), or a pharmaceutically acceptable salt thereof. In certain embodiments, the PRMT5-mediated disorder is a proliferative disorder, a metabolic disorder, or a blood disorder. In certain embodiments, compounds described herein are useful for treating cancer. In certain embodiments, compounds described herein are useful for treating hematopoietic cancers, lung cancer, prostate cancer, melanoma, or pancreatic cancer. In certain embodiments, compounds described herein are useful for treating a hemoglobinopathy. In certain embodiments, compounds described herein are useful for treating sickle cell anemia. In certain embodiments, compounds described herein are useful for treating diabetes or obesity.

Compounds described herein are also useful for the study of PRMT5 in biological and pathological phenomena, the study of intracellular signal transduction pathways mediated by PRMT5, and the comparative evaluation of new PRMT5 inhibitors.

This application refers to various issued patent, published patent applications, journal articles, and other publications, all of which are incorporated herein by reference.

Definitions of specific functional groups and chemical terms are described in more detail below. The chemical elements are identified in accordance with the Periodic Table of h Ed., the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The “active site” of an enzyme refers to the catalytic site of the enzyme (i.e., where the reaction catalyzed by the enzyme occurs). For example, in a methyltransferase such as PRMT5, the active site is where the transfer of the methyl group from SAM is transferred to the arginine of a histone protein occurs. The structure and chemical properties of the active site typically allow the recognition and binding of a substrate. The active site typically includes residues responsible for the binding specificity (e.g., charge, hydrophobicity, and/or steric hindrance) and catalytic residues of the enzyme.

In one aspect, the active site of PRMT5 comprises amino acids Leu319, Phe327, Glu435, Leu437, Glu444, and Phe580. In some embodiments, the active site of PRMT5 comprises amino acids Leu319, Phe327, Lys333, Glu435, Leu437, Gly438, Ser439, Glu444, Val503, Ser578, Trp579, and Phe580. In some embodiments, the active site of PRMT5 comprises amino acids Leu312, Leu319, Thr323, Tyr324, Phe327, Glu328, Lys333, Tyr334, Glu435, Leu437, Gly438, Ser439, Glu444, Val503, Ser578, Trp579, Phe580, and Pro581. In some embodiments, the active site of PRMT5 comprises amino acids Leu319, Phe327, Lys333, Glu435, Leu437, Gly438, Ser439, Glu444, Val503, Phe577, Ser578, Trp579, and Phe580. In some embodiments, the active site of PRMT5 comprises amino acids Phe300, Tyr304, Gln309, Ser310, Pro311, Leu312, Leu319, Phe327, Lys333, Glu435, Leu437, Gly438, Ser439, Glu444, Val503, Ser578, Trp579, and Phe580. In some embodiments, the active site of PRMT5 comprises amino acids Phe300, Leu312, Leu319, Gln322, Thr323, Tyr324, Val326, Phe327, Glu328, Lys333, Tyr33, Glu435, Leu437, Gly438, Ser439, Glu444, Val503, Phe577, Ser578, Trp579, Phe580, and Pro581. In some embodiments, the active site of PRMT5 comprises amino acids Phe300, Tyr304, Gln309, Ser310, Pro311, Leu312, Leu319, Thr323, Tyr324, Val326, Phe327, Glu328, Lys333, Tyr33, Glu435, Leu437, Gly438, Ser439, Glu444, Val503, Phe577, Ser578, Trp579, and Phe580. It should further be appreciated that one or more cofactors may also be present in the active site. In some embodiments, the cofactor is S-adenosyl methionine (SAM), or an analog thereof.

Amino acid residues in proteins or peptides are abbreviated as follows: phenylalanine is Phe or F; leucine is Leu or L; isoleucine is Ile or I; methionine is Met or M; valine is Val or V; serine is Ser or S; proline is Pro or P; threonine is Thr or T; alanine is Ala or A; tyrosine is Tyr or Y; histidine is His or H; glutamine is Gln or Q; asparagine is Asn or N; lysine is Lys or K; aspartic acid is Asp or D; glutamic Acid is Glu or E; cysteine is Cys or C; tryptophan is Trp or W; arginine is Arg or R; and glycine is Gly or G. For further description of amino acids, see Proteins: Structure and Molecular Properties by Creighton T. E. (1983), W. H. Freeman & Co., New York, incorporated herein by reference.

The term “atomic coordinates” refers to mathematical coordinates derived from mathematical equations related to the patterns obtained on diffraction of a monochromatic beam of x-rays by the atoms (scattering centers) of a protein molecule in crystal form. The diffraction data are used to calculate an electron density map of the repeating unit of the crystal. The electron density map is then used to establish the positions of the individual atoms within the unit cell of the crystal. The coordinates can also be obtained by the aid of computational analysis.

As used herein, a “binding compound” refers to a compound that reversibly or irreversibly binds to PRMT5. In certain embodiments, the binding compound binds in the active site of PRMT5. A binding compound may be an inhibitor of PRMT5 (e.g., eliciting inhibition or reduction in enzymatic activity) or an activator of PRMT5 (e.g., eliciting an increase in enzymatic activity). In certain embodiments, a small molecule binding compound is of Formula (I):

Ring AA-M-Ring BB.

By “choosing” is meant picking a chemical compound from a chemical library or commercially available source.

By “design” or “designing” is meant to provide a novel molecular structure of, for example, a compound, such as a small molecule, or a polypeptide or nucleic acid that has desired properties or characteristics.

By “identify” or “identifying” is meant to determine a condition, compound, polypeptide, amino acid, or nucleic acid that corresponds to or exhibits a desired characteristic or property.

As used herein the term “inhibit” means to reduce the amount of PRMT5 activity to a level or amount that is statistically significantly less than an initial level, which may be a baseline level of PRMT5 activity.

The term “modulate,” as used herein, means to increase or decrease PRMT5 enzymatic activity.

By “screen” or “screening” is meant to test for in silico, in vitro, or in vivo a compound with a particular characteristic or desired property. These characteristics or desired properties may be chemical, biological, or physical in nature or a combination thereof. For example, in screening for PRMT5 binding compounds the desired characteristics may include, but are not limited to, high affinity intracellular binding to PRMT5, high specificity for binding to one or multiple binding sites on PRMT5, low specificity for binding to one or multiple binding sites on PRMT5, high degree of inhibition of PRMT5 activity, high bioavailability of the compound, efficient cellular uptake of the compound, high solubility of the compound in pharmacological carriers, low pharmacological toxicity of the compound, etc. Screening may be performed in vitro or in vivo using compound libraries, such as small molecule libraries, peptide libraries, DNA libraries, or RNA libraries. Screening in silico may be performed using predefined or randomized screening parameters and data sets, for example, of known test compounds and/or test conditions.

By “select” or “selecting” is meant to provide a pre-existing molecular structure and to choose, for example, from a group of pre-existing compounds, such as a small molecules, polypeptides, or nucleic acids one or more members that have or exhibit a desired property or characteristic.

The term “subject,” as used herein, refers to any animal. In certain embodiments, the subject is a mammal. In certain embodiments, the term “subject”, as used herein, refers to a human (e.g., male, female, adult, or child). The subject may be at any stage of development. The subject may be a transgenic animal and/or experimental animal, e.g. a mammal (mouse, rat, hamster, pig, goat, cow, camel, sheep, cat, dog, etc.), a fish (zebrafish etc.), a nematode (Caenorhabditis elegans etc.), an insect (Drosophila melanogaster etc.), a frog (Xenopus laevis).

By the term “synthesizing” is meant making a chemical structure from precursors by chemical processes. Synthesizing implies making at least one compound, but is not limited to one compound. In certain aspects, synthesizing implies making more than one compound, such as a series of compounds synthesized in an effort to study structure-activity relationships (SAR) using standard chemistry methods, and/or a series of structurally similar compounds made using standard combinatorial techniques.

It will be appreciated that compounds that modulate PRMT5 activity may have chemical structures that can be altered. For example, one or more substituents of identified compounds may be substituted with any number of other substituents or functional moieties. When more than one position in any given structure may be substituted with more than one substituent selected from a specified group, the substituent may be either the same or different at every position. As used herein, the term “substituted” is contemplated to include substitution with all permissible substituents of organic compounds, any of the substituents described herein (for example, aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, etc.), and any combination thereof (for example, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like) that results in the formation of a stable moiety. Heteroatoms such as nitrogen may have hydrogen substituents and/or any suitable substituent as described herein which satisfy the valencies of the heteroatoms and results in the formation of a stable moiety.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds of the present invention may be depicted as different tautomers. It should also be understood that when compounds have tautomeric forms, all tautomeric forms are intended to be included in the scope of the present invention, and the naming of any compound described herein does not exclude any tautomer form.

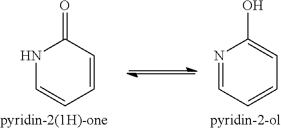 [SMILES: O=c1cccc[nH]1 ; Oc1ccccn1]

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “aliphatic,” as used herein, includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups. As will be appreciated by one of ordinary skill in the art, “aliphatic” is intended herein to include alkyl, alkenyl, alkynyl, cycloalkyl, and cycloalkenyl moieties.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. In certain embodiments, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In certain embodiments, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. In certain embodiments, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (Cₘ), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In certain embodiments, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). In certain embodiments, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl, and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 it electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. In certain embodiments, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 it electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. In certain embodiments, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” aliphatic, “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, including any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{aa})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R)₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two e groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

These and other exemplary substituents are described in more detail in the Detailed Description, Examples, and claims. The present disclosure is not intended to be limited in any manner by the above exemplary listing of substituents.

### OTHER DEFINITIONS

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

A “subject” to which administration is contemplated includes, but is not limited to, humans (e.g., a male or female of any age group, e.g., a pediatric subject (e.g, infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), rodents (e.g., rats and/or mice), reptiles, amphibians, and fish. In certain embodiments, the non-human animal is a mammal. The non-human animal may be a male or female at any stage of development. A non-human animal may be a transgenic animal.

“Condition,” “disease,” and “disorder” are used interchangeably herein.

“Treat,” “treating” and “treatment” encompasses an action that occurs while a subject is suffering from a condition which reduces the severity of the condition or retards or slows the progression of the condition (“therapeutic treatment”). “Treat,” “treating” and “treatment” also encompasses an action that occurs before a subject begins to suffer from the condition and which inhibits or reduces the severity of the condition (“prophylactic treatment”).

An “effective amount” of a compound refers to an amount sufficient to elicit the desired biological response, e.g., treat the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, the term “methyltransferase” represents transferase class enzymes that are able to transfer a methyl group from a donor molecule to an acceptor molecule, e.g., an amino acid residue of a protein or a nucleotide of a DNA molecule. Methytransferases typically use the reactive methyl group bound to sulfur in S-adenosyl methionine (SAM) as the methyl donor. In some embodiments, a methyltransferase described herein is a protein methyltransferase. In some embodiments, a methyltransferase described herein is a histone methyltransferase. Histone methyltransferases (HMT) are histone-modifying enzymes, (including histone-lysine N-methyltransferase and histone-arginine N-methyltransferase), that catalyze the transfer of one or more methyl groups to lysine and arginine residues of histone proteins. In certain embodiments, a methyltransferase described herein is a histone-arginine N-methyltransferase.

Protein arginine methyltransferase 5 (PRMT5) catalyzes the addition of two methyl groups to the two w-guanidino nitrogen atoms of arginine, resulting in ω-NG, N′G symmetric dimethylation of arginine (sDMA) of the target protein. PRMT5 functions in the nucleus as well as in the cytoplasm, and its substrates include histones, spliceosomal proteins, transcription factors (See e.g., Sun et al., 2011, PNAS 108: 20538-20543). PRMT5 generally functions as part of a molecule weight protein complex. While the protein complexes of PRMT5 can have a variety of components, they generally include the protein MEP50 (methylosome protein 50). In addition, PRMT5 acts in conjunction with cofactor SAM (S-adenosyl methionine).

It was surprisingly found that compounds with specific structural elements can bind PRMT5 and are effective in inhibiting the biological function of PRMT5 (e.g., have a low IC₅₀ value). It is shown herein that compounds that have two aromatic ring structures separated by a flexible linker can inhibit the function of PRMT5 if the compound can engage in certain interactions with amino acids in the active site of PRMT5. In some embodiments, the disclosure provides compounds of the formula Ring AA-M-Ring BB, wherein Ring AA and Ring BB are aromatic ring systems, and wherein M is a linker that allows for the planes of Ring AA and Ring BB to be at about 90° relative to each other. As shown herein, compounds of the formula Ring AA-M-Ring BB that can minimally interact with certain moieties (e.g., SAM and certain amino acids) in the active site of PRMT5 can inhibit the function of PRMT5. In some embodiments compounds of the formula Ring AA-M-Ring BB can form a pi-cation interaction with one or more amino acids in the active site of PRMT5.

In some embodiments Ring BB can form a pi-cation interaction with one or more amino acids in the active site of PRMT5. In some embodiments compounds of the formula Ring AA-M-Ring BB can form a pi-stacking interaction with one or more amino acids in the active site of PRMT5. In some embodiments Ring BB can form a pi-stacking interaction with one or more amino acids in the active site of PRMT5. In some embodiments, the compounds interact with SAM or Phe327 of the active site of PRMT5. In some embodiments, the compounds interact with SAM and Phe327 of the active site of PRMT5. In some embodiments, as provided herein, compounds of the formula Ring AA-M-Ring BB can undergo a cation-pi interaction with SAM through the aromatic ring system in Ring BB. In some embodiments, as provided herein, compounds of the formula Ring AA-M-Ring BB can interact with Phe327 (e.g., through pi stacking) through the aromatic ring system in Ring BB. In some embodiments, as provided herein, compounds of the formula Ring AA-M-Ring BB can inhibit the action of PRMT5 because Ring BB interacts with SAM and Phe327 and because the flexible linker allows for the second aromatic ring system (Ring AA) to interact with other residues in the active site of PRMT5 (e.g., Phe580). In some embodiments, such compounds can inhibit PRMT5 with an IC₅₀ of 100 nM or lower. In some embodiments, such compounds can inhibit PRMT5 with an IC₅₀ of 50 nM or lower. In some embodiments, such compounds can inhibit PRMT5 with an IC₅₀ of 10 nM or lower. In some embodiments, such compounds can inhibit PRMT5 with an IC₅₀ of 1 nM or lower.

In one aspect, the disclosure provides a compound that can bind PRMT5, wherein the compound has the formula:

Ring AA-M-Ring BB;

wherein

Ring AA is an optionally substituted aromatic moiety;

M is an aliphatic linker; and

Ring BB is an aromatic moiety capable of undergoing a pi-cation interaction with S-adenosyl methionine (SAM) and capable of undergoing a pi-stacking interaction with Phe327 of PRMT5;

wherein the planes of Ring A and Ring B are at an angle between 75° and 105° relative to each other. In some embodiments, the compound has an IC₅₀ for PRMT5 of less than 100 nM.

In addition to interacting with SAM and Phe327 the Ring AA-M-Ring BB compounds provided herein may interact with additional amino acids in the active site of PRMT5 such as Leu319, Glu435, Leu437, Glu444, and Phe580.

In some embodiments, the active site of PRMT5 comprises the amino acids Leu319, Phe327, Glu435, Leu437, Glu444, and Phe580. In addition, it should be appreciated that cofactors such as SAM or SAM analogs (e.g., SAH) may also be present in the active site of PRMT5. The numbering of the PRMT5 amino acids as provided herein relates to human PRMT5 isoform A (SEQ ID NO:1) depicted (in part) in Table A. However, it should be appreciated that the interactions between the compounds of formula Ring AA-M-Ring BB provided herein and the active site of PRMT5 should be applicable to homologous versions of PRMT5, just as long as the active site is conserved. The term “active site” (or “binding pocket”) relates to the three-dimensional structure of the portion of the protein responsible for the methyltransferase activity, e.g., the region of the protein that acts on the substrate(s) of the protein or catalyze the transfer of a methyl group from SAM to a protein substrate.

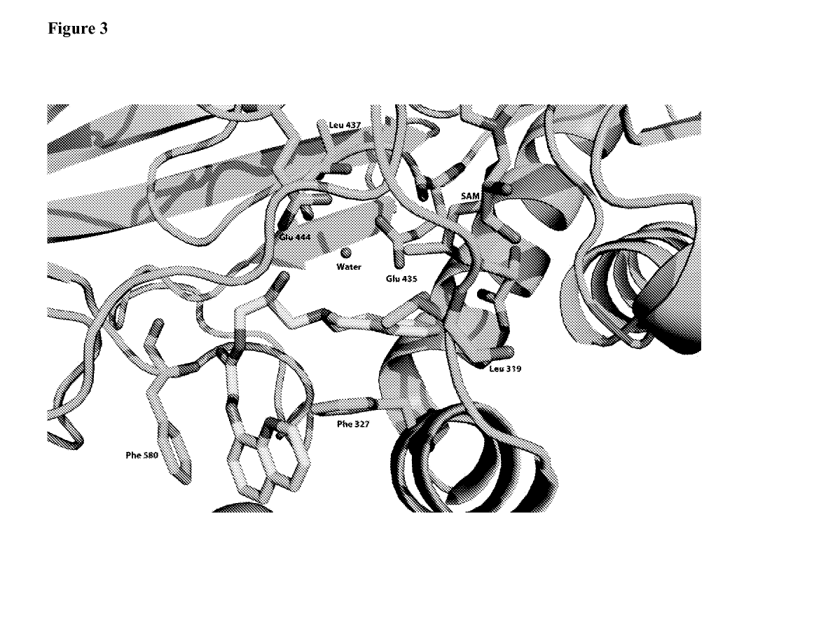

In some embodiments, the active site of PRMT5 comprises the amino acids Leu319, Phe327, Glu435, Leu437, Glu444, and Phe580. In some embodiments, the Ring AA-M-Ring BB compounds described herein interact with glutamic acid residues Glu435 and Glu444. In certain embodiments, the Ring BB portion of the compound interacts with Glu435 and Glu444. In some embodiments, the linker M interacts with Glu435 and Glu444. In certain embodiments, the Ring BB portion of the compound and the linker interact with Glu435 and Glu444. In some embodiments, the interaction of the compounds with glutamic acid residues Glu435 and Glu444 is in addition to the pi-cation interaction with SAM and the pi-stacking interaction with Phe327. In some embodiments, the interaction of the compound with Glu435 and Glu444 is a hydrogen bonding interaction. In some embodiments, the interaction of the compound with Glu435 and Glu444 is mediated through a water molecule. In some embodiments, the Ring BB of the compounds provided herein comprises a heteroatom such as a nitrogen atom. In some embodiments, the nitrogen atom is tetrahedrally coordinated. In some embodiments, the nitrogen atom of Ring BB interacts with the Glu435 and Glu444, either directly or through a coordinated water molecule (See e.g., FIG. 3).

In some embodiments, compounds of the formula Ring AA-M-Ring BB described herein interact with Leu437. In some embodiments, the Ring AA-M-Ring BB compounds described herein interact with the carbonyl group of Leu437. In some embodiments, the Ring BB of the compounds provided herein comprises a heteroatom such as a nitrogen atom. In some embodiments, the nitrogen atom of Ring BB is tetrahedrally coordinated. In some embodiments, the nitrogen interacts with the carbonyl group of Leu437, either directly or through a coordinated water molecule. In some embodiments, the nitrogen interacts with the Leu437, Glu435 and Glu444, either directly or through a coordinated water molecule. In some embodiments, the linker M interacts with Leu437.

In some embodiments, the active site of PRMT5 comprises SAM and the amino acids Leu319, Phe327, Glu435, Leu437, Glu444, and Phe580. In some embodiments, compounds of the formula Ring AA-M-Ring BB described herein interact with Leu319. In some embodiments Ring BB interacts with Leu319. In some embodiments, the interaction with Leu319 is in addition to the interaction with one or more of the interactions of the compound with SAM and amino acids Phe327, Glu435, Leu437 and Glu444.

In some embodiments, compounds of the formula Ring AA-M-Ring BB described herein interact with Phe580. In some embodiments, Ring AA of the compound interacts with Phe580. In some embodiments, the interaction with Phe580 is in addition to the interaction with one or more of the interactions of the compound with SAM and amino acids Leu319, Phe327, Glu435, Leu437 and Glu444.

In some embodiments, compounds of the formula Ring AA-M-Ring BB described herein interact with SAM and Phe327 through Ring BB and with Phe580 through Ring AA. The compounds described herein can interact with SAM and Phe327 through Ring BB and with Phe580 through Ring AA at the same because the linker M allows for the planes of Ring AA and Ring BB to be at an angle between 75° and 105°. In some embodiments, the angle of the planes of Ring AA and Ring BB is between 80° and 100°. In some embodiments, the angle of the planes of Ring AA and Ring BB is between 85° and 95°. In some embodiments, the angle of the planes of Ring AA and Ring BB is 70°, 71°, 72°, 73°, 74°, 75°, 76°, 77°, 78°, 79 °, 80°, 81°, 82°, 83°, 84°, 85°, 86°, 87°, 88°, 89°, 90°, 91°, 92°, 93°, 94°, 95°, 96°, 97°, 98°, 99°, 100°, 101°, 102°, 103°, 104°, 105°, 106°, 107°, 108°, 109°, or 110°.

In some embodiments the linker M is a flexible linker. In some embodiments, linker M is unbranched. In some embodiments, linker M is flexible enough to allow Ring BB of the compound to get sufficiently close to SAM to engage in a pi-cation interaction.

In some embodiments, the disclosure provides a compound of the formula Ring AA-M-Ring BB wherein the planes of Ring AA and Ring BB are at an angle of between 75° and 105° when the compound is bound to PRMT5. In some embodiments, the angle of the planes of Ring AA and Ring BB is between 80° and 100° when the compound is bound to PRMT5. In some embodiments, the angle of the planes of Ring AA and Ring BB is between 85° and 95° when the compound is bound to PRMT5. In some embodiments, the disclosure provides a compound of the formula Ring AA-M-Ring BB wherein the planes of Ring AA and Ring BB are at an angle of between 75° and 105° when the compound is co-crystallized with PRMT5. In some embodiments, the angle of the planes of Ring AA and Ring BB is between 80° and 100° when the compound is co-crystallized with PRMT5. In some embodiments, the angle of the planes of Ring AA and Ring BB is between 85° and 95° when the compound is co-crystallized with PRMT5.

In some embodiments, the active site of PRMT5 is identified by amino acids Leu319, Phe327, Lys333, Glu435, Leu437, Gly438, Ser439, Glu444, Val503, Ser578, Trp579, and Phe580. In some embodiments, the active site of PRMT5 further includes SAM or a SAM analog. In some embodiments, compounds of the formula Ring AA-M-Ring BB described herein interact with SAM, Phe327, and one or more additional amino acids selected from the group consisting of amino acids Leu319, Lys333, Glu435, Leu437, Gly438, Ser439, Glu444, Val503, Ser578, Trp579, and Phe580.

In some embodiments, the active site of PRMT5 is identified by amino acids Leu312, Leu319, Thr323, Tyr324, Phe327, Glu328, Lys333, Tyr334, Glu435, Leu437, Gly438, Ser439, Glu444, Val503, Ser578, Trp579, Phe580, and Pro581. In some embodiments, the active site of PRMT5 further includes SAM or a SAM analog. In some embodiments, compounds of formula Ring AA-M-Ring BB described herein interact with SAM, Phe327, and one or more additional amino acids selected from the group consisting of amino acids Leu312, Leu319, Thr323, Tyr324, Glu328, Lys333, Tyr334, Glu435, Leu437, Gly438, Ser439, Glu444, Val503, Ser578, Trp579, Phe580, and Pro581.

As disclosed herein, compounds of formula Ring AA-M-Ring BB described herein inhibit the enzymatic activity of PRMT5 (i.e., have a low IC₅₀ value). It is believed that the low IC₅₀ is due to the well-defined interactions within the active site the compounds can engage in. Thus, it is believed that the Ring AA-M-Ring BB compounds described herein have a low IC₅₀ because they can engage in a stabilizing pi-cation interaction with SAM and pi-stacking interaction with Phe327. In certain embodiments, a provided compound inhibits PRMT5 at an IC₅₀ less than or equal to 10 μM. In certain embodiments, a provided compound inhibits PRMT5 at an IC₅₀ less than or equal to 1 μM. In certain embodiments, a provided compound inhibits PRMT5 at an IC₅₀ less than or equal to 0.1 μM. In certain embodiments, a provided compound inhibits PRMT5 at an IC₅₀ less than or equal to 50 nM. In certain embodiments, a provided compound inhibits PRMT5 at an IC₅₀ less than or equal to 10 nM. In certain embodiments, a provided compound inhibits PRMT5 at an IC₅₀ less than or equal to 1 nM. In certain embodiments, a provided compound inhibits PRMT5 in a cell at an EC₅₀ less than or equal to 10 μM. In certain embodiments, a provided compound inhibits PRMT5 in a cell at an EC₅₀ less than or equal to 1 μM. In certain embodiments, a provided compound inhibits PRMT5 in a cell at an EC₅₀ less than or equal to 0.1 μM. In certain embodiments, a provided compound inhibits PRMT5 in a cell at an EC₅₀ less than or equal to 50 nM. In certain embodiments, a provided compound inhibits PRMT5 in a cell at an EC₅₀ less than or equal to 10 nM. In certain embodiments, a provided compound inhibits PRMT5 in a cell at an EC₅₀ less than or equal to 1 nM. In certain embodiments, a provided compound inhibits cell proliferation at an EC₅₀ less than or equal to 10 μM. In certain embodiments, a provided compound inhibits cell proliferation at an EC₅₀ less than or equal to 1 μM. In certain embodiments, a provided compound inhibits cell proliferation at an EC₅₀ less than or equal to 0.1 μM. In certain embodiments, a provided compound inhibits cell proliferation at an EC₅₀ less than or equal to 20 nM. In certain embodiments, a provided compound inhibits cell proliferation at an EC₅₀ less than or equal to 10 nM. In certain embodiments, a provided compound inhibits cell proliferation at an EC₅₀ less than or equal to 1 nM. In certain embodiments, the IC₅₀ and EC₅₀ values are calculated according to the methods provided in the Examples herein.

In one aspect, the disclosure provides PRMT5 inhibitors with the shape of the PRMT5-active site. In some embodiments, the PRMT5 active site is defined by amino acids Leu319, Phe327, Glu435, Leu437, Glu444, and Phe580. In some embodiments, the PRMT5 active site is defined by and amino acids Leu319, Phe327, Lys333, Glu435, Leu437, Gly438, Ser439, Glu444, Val503, Ser578, Trp579, and Phe580. In some embodiments, the PRMT5 active site is defined by and amino acids Leu312, Leu319, Thr323, Tyr324, Phe327, Glu328, Lys333, Tyr334, Glu435, Leu437, Gly438, Ser439, Glu444, Val503, Ser578, Trp579, Phe580, and Pro581. It should be appreciated that the active site may also include a SAM or an analog of SAM. Thus, for instance, the active site may include SAH (S-adenosyl-L-homocysteine). The active site may also include sinefungin (another SAM analog). Additional SAM analogs are known in the art and are described for instance in Peterli-Roth et al. (J. Org. Chem 1994: 59, 4186-4193).

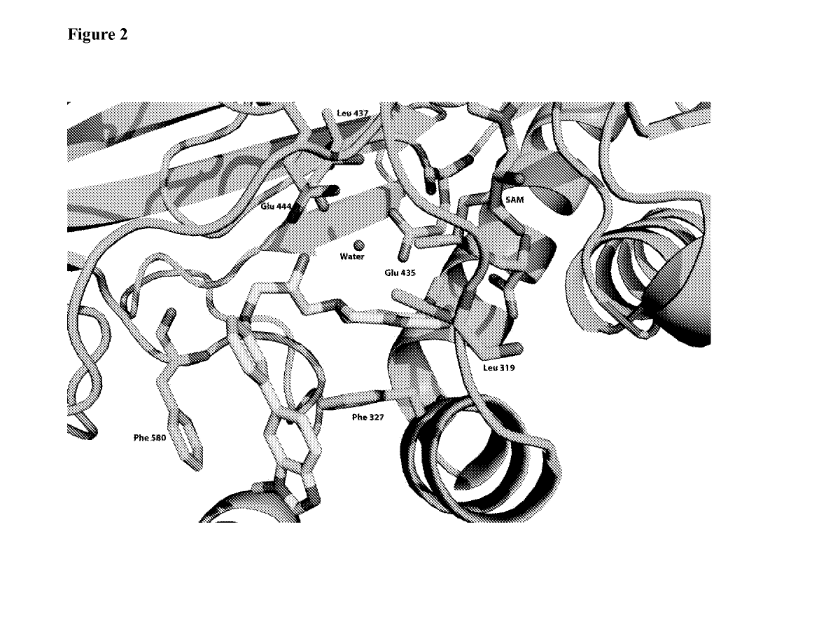

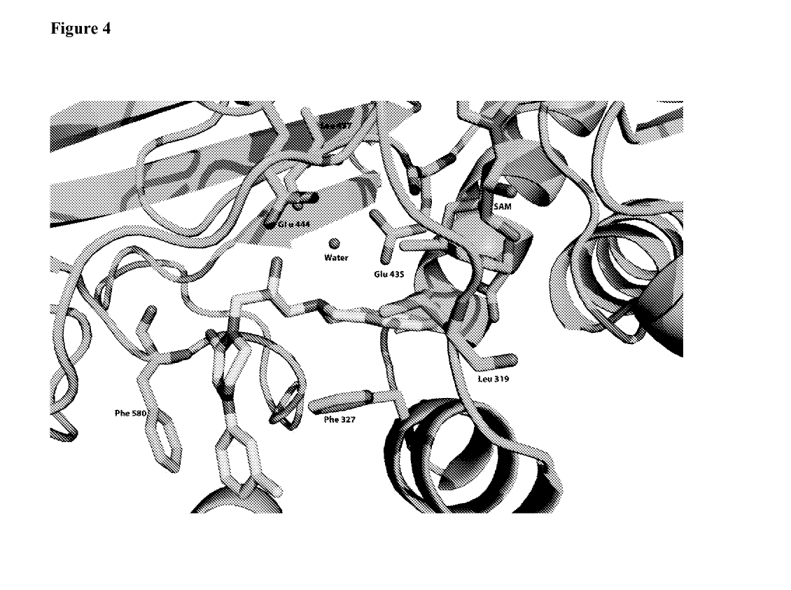

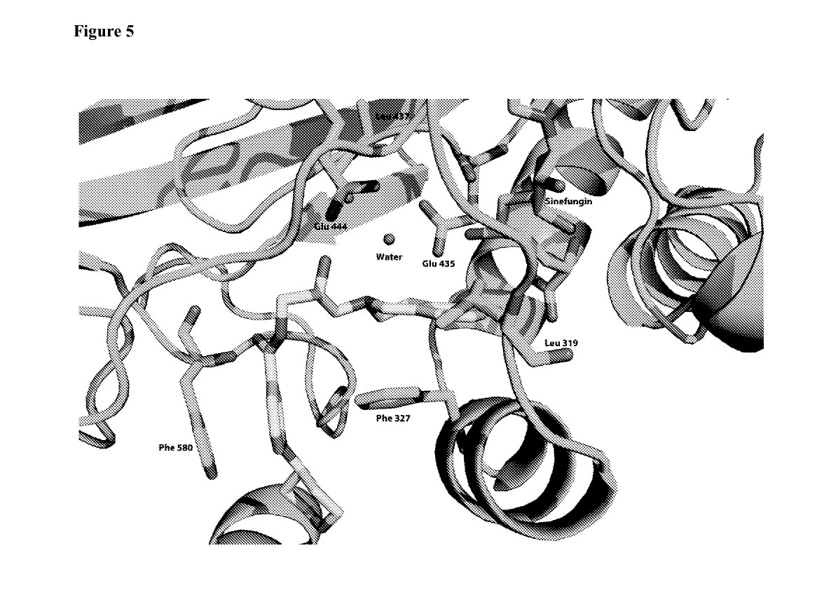

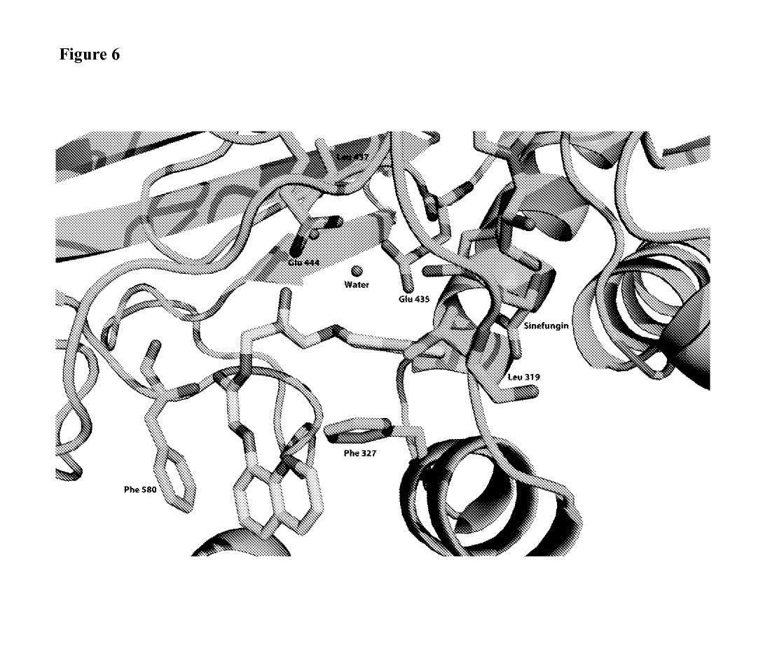

In some embodiments, the active site is defined by S-adenosyl methionine (SAM) and amino acids Leu319, Phe327, Glu435, Leu437, Glu444, and Phe580, according to the atomic coordinates in Table A provided herein. Table A provides the coordinates of an exemplary crystal structure of the complex PRMT5-SAM-compound 6A. In addition, FIG. 3 provides a representation of the active site, including SAM, and amino acids Leu319, Phe327, Glu435, Leu437, Glu444, and Phe580, based on the coordinates provided in Table A. FIGS. 2 and 4-6 provide additional examples of representations of compounds of formula Ring AA-M-Ring BB as described herein based on the coordinates of co-crystals of these compounds with PRMT5 (Compound FIG. 2, Compound FIG. 4, Compound FIG. 5, Compound FIG. 6, depicted below). As can be seen in the Figures all active sites have a similar geometry. It is also evident from the figures that the five exemplary compounds of formula Ring AA-M-Ring BB all show similar binding motifs. Thus, the compounds are characterized by a pi-cation interaction between Ring BB and SAM (or sinefungin or other SAM analog) and a pi-stacking interaction with Phe327. In addition, all compounds show the geometry of having planes of Rings A and B that are between 75° and 105° relative to each other. The compounds also show the interaction of the nitrogen of Ring AA with Glu435, Leu437, and Glu444, the interaction of the compounds with Leu319 and the interaction of Ring AA with Phe580.

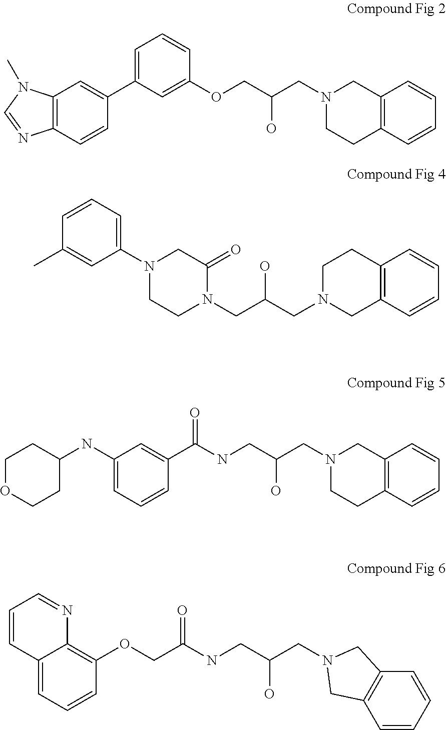 [SMILES: Cn1cnc2ccc(-c3cccc(OCC(O)CN4CCc5ccccc5C4)c3)cc21 ; Cc1cccc(N2CCN(CC(O)CN3CCc4ccccc4C3)C(=O)C2)c1 ; O=C(NCC(O)CN1CCc2ccccc2C1)c1cccc(NC2CCOCC2)c1 ; O=C(COc1cccc2cccnc12)NCC(O)CN1Cc2ccccc2C1]

In one aspect, the disclosure provides compositions comprising PRMT5 and a compound of the formula Ring AA-M-Ring BB as described herein. In some embodiments, the disclosure provides a composition comprising PRMT5 and a compound of the formula:

Ring AA-M-Ring BB;

wherein

Ring AA is an optionally substituted aromatic moiety;

M is an aliphatic linker; and

Ring BB is an aromatic moiety capable of undergoing a pi-cation interaction with SAM of the PRMT5-SAM complex and capable of undergoing a pi-stacking interaction with Phe327 of PRMT5;

wherein the planes of Ring A and Ring B are at an angle between 75° and 105° relative to each other. In some embodiments, the compound has an IC₅₀ for PRMT5 of less than 100 nM. In some embodiments, the compound has an IC₅₀ for PRMT5 of less than 50 nM. In some embodiments, the compound has an IC₅₀ for PRMT5 of less than 10 nM. In some embodiments, the compound has an IC₅₀ for PRMT5 of less than 1 nM. In some embodiments, the compositions also includes a co-factor, such as SAM.

The composition comprising PRMT5 and the compound of the formula Ring AA-M-Ring BB may include additional components such as additional proteins, including proteins with which PRMT5 is associated intracellularly (e.g., MEP50). In some embodiments, the complex is isolated or partially isolated. An isolated form of the complex, for instance, is a complex comprising PRMT5 and a compound as the main components, optionally with buffers, salts, etc. in addition to the main components. Such complexes are formed for instance in vitro. In some embodiments, an isolated complex is a complex purified from the cell. In some embodiments, an isolated complex does not include any major cellular components except for PRMT5. However, it should be appreciated that the complexes may be administered as a complex, for instance to compete out active PRMT5 with the inactive complexed PRMT5. In some embodiments, the complex is in vivo (e.g., intracellular and/or in a subject). The complex may be formed for instance upon addition of the compound of the formula Ring AA-M-Ring to a subject or cell resulting in the formation of the complex in vivo. Because of the ability of the compounds to inhibit the enzymatic activity of PRMT5 (e.g., IC₅₀<100 nM), it is expected that the complexes provide herein will be particularly stable. The affinity of the compound for PRMT5 is typically correlated to the IC₅₀.

### Crystal Structure of PRMT5

In certain embodiments, the present invention provides three-dimensional structural information for PRMT5 or for PRMT5 variants that comprise one or more amino acid substitutions, deletions, or duplications. In some embodiments, the invention provides methods for constructing models of these variants using the three-dimensional structural information for PRMT5 as a template. The method may include adjusting the backbone dihedral angles and the side chains of each amino acid that is modeled until a low energy conformation is obtained. (e.g., by using AMoRe, Phaser, MolRep or other crystallography software programs)

In certain embodiments, X-ray diffraction data collection is performed in an X-ray crystallography facility. One, two, three, or more diffraction data sets may be collected from one or more PRMT5 crystals. In certain embodiments, the crystals of the present invention diffract to a resolution limit of at least approximately 8 angstrom (Å). In certain embodiments, the crystals diffract to a resolution limit of at least approximately 6 Å. In certain embodiments, the crystals diffract to a resolution limit of at least approximately 4 Å. In certain embodiments, the crystals diffract to a resolution limit of at least approximately 2.5 Å. In certain embodiments, the crystal diffracts x-rays for a determination of structural coordinates to a maximum resolution of about 3.9 Å, of about 3.2 Å, or of about 2.9 Å. The crystals may diffract to a maximum resolution of about 2.5 Å to about 3.5 Å, of about 2.0 Å to about 3.0 Å, of about 2.5 Å to about 3.0 Å, or of about 3.0 Å to about 3.5 Å.

Diffraction data can be collected at variable oscillation angles, number of frames and exposure times that all depend on the equipment used and on the quality of the crystal(s) used to collect the data. One of ordinary skill would know how to optimize these parameters (Principles of protein X-ray crystallography by J. Drenth. 2nd ed. (1999) Springer-Verlag, Heidelberg, Germany; Structure Determination by X-ray Crystallography by M. Ladd and R. Palmer. 4th ed. (2003) Kluwer Academic/Plenum Publishers, New York, N.Y.). In certain embodiments, diffraction data can be collected with 1° oscillation. Other oscillation may be used, e.g. oscillations of less than or greater than 1°. For example, diffraction data can be collected with 0.1°, 0.3°, 0.5°, 1°, 1.5°, 2°, 3°, 4°, 5°, or 10° oscillation, or any oscillation angle in between these angles. In certain embodiments, 120 frames are collected. More or fewer than 120 frames may be collected. For example, 10, 20, 50, 100, 200, 300. 400, 500, 1000, or 5000 frames may be collected, or any number of frames in between these numbers. In certain embodiments, the exposure is 5 minutes per frame. Other frame exposure times may also be used, such as, for example 5 seconds, 10 seconds, 20 seconds, 30 seconds, 40 seconds, 50 seconds, 60 seconds, 120 seconds, 180 seconds, 3 minutes, 4 minutes, 10 minutes, 20 minutes, 30 minutes per frame or any exposure time in between these times. Data merging and scaling can be done, for example, using HKL2000 software suite (HKL Research, Inc., Charlottesville, Va.). Structure determination, model building, and refinement can be performed, for example, using software such as Molrep, coot and Refmac that are part of CCP4 software suite. MolRep is a program for automated molecular replacement (e.g., MolRep, version 10.2.35). Coot Graphical Interface by Paul Emsley (www.ysbl.york.ac.uk/˜emsley) for model building includes an interface to refmac5 (Gnu Public License; refmac5, e.g. version 5.5.0072 or version 5.5.0109). A macromolecular refinement program by Garib Murshudov et al. is integrated into the CCP4 program suite (www.ccp4.ac.uk, CCP4, version 6.1.3). Structural analyses may be performed using molecular viewer software PYMOL (pymol.org).

In certain embodiments, the atomic coordinates of crystalline PRMT5 are provided. In some embodiments, the coordinates are provides on a computer readable medium or in the memory of a computer.

The three-dimensional structure of the active site of PRMT5 is provided by the atomic coordinates listed in Table A.

It should be understood that while Table A provides atomic coordinates for crystalline PRMT5, the present invention also contemplates structural modifications thereof, for example, as having significant structural homology (e.g., significant structural overlap), particularly in the areas recognized as active, and thus providing the same or similar structural information as provided herewith. Significant structural homology refers to at least one of the following criteria: (i) at least 60%, 65%, 70%, 75%, 80%, 85%, 90%, 95%, or 99% structural homology with crystalline PRMT5; or (ii) at least 60%, 65%, 70%, 75%, 80%, 85%, 90%, 95%, or 99% structural homology with a recognized active binding site of crystalline PRMT5. In certain embodiments, significant structural homology may also refer to at least 60%, 65%, 70%, 75%, 80%, 85%, 90%, 95%, or 99% structural homology with the primary amino acid sequence of PRMT5. Furthermore, the primary amino acid sequence of PRMT5 may be a sequence included as a segment in a larger amino acid sequence, or may be a fragment thereof. In some embodiments, a fragment of a full-length, wild-type PRMT5 protein is provided or used in an inventive method or system provided herein. In some embodiments, a PRMT5 fragment comprises a PRMT5 sequence of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50-75, 75-100, 100-150, 150-200, 200-250, 250-500, or more than 500 amino acids. In some embodiments, a fragment of PRMT5 does not comprise a full-length PRMT5 sequence, for example, a full-length human PRMT5 sequence. In some embodiments, a fragment of PRMT5 comprises all or at least part of the protein responsible for the enzymatic activity of full-length PRMT5.

### Uses of PRMT5 Structural Information

In another aspect the invention provides methods and/or uses of PRMT5 structural information, for example, and methods for designing, identifying, and/or screening binding compounds to PRMT5 that may be useful in treating a disease. In certain embodiments, methods for designing, identifying, and/or screening binding compounds to PRMT5 (e.g. wild-type and/or mutated PRMT5) are provided. The compound may also affect aspects of intracellular trafficking of PRMT5 or aspects of enzymatic function, such as substrate recognition and/or PRMT5 catalytic activity. In certain embodiments, methods are provided for the in silico design, identification, and/or screening of PRMT5 binding compounds using the three-dimensional structural information provided herein. In certain embodiments, methods are provided that can be used to identify inhibitors, reversible inhibitors, and/or activators of PRMT5 activity. In certain embodiments, methods are provided that can be used to identify binding compounds. In certain embodiments, methods are provided that can be used to identify binding compounds that modulate PRMT5 activity. In certain embodiments, methods are provided that can be used to test potential binding compounds for their ability to modulate PRMT5 activity. In certain embodiments, these methods include in silico, in vitro, and in vivo methods. In certain embodiments, methods are provided, solving the structure of PRMT5 homologs or orthologs using the three-dimensional structural information provided herein. In certain embodiments, methods are provided, solving the (partial) structure of proteins comprising structurally or functionally homologous domains using the three-dimensional structural information for PRMT5 provided herein.

In certain embodiments, the present disclosure provides PRMT5 inhibitors having molecular dimensions compatible with the shape of a PRMT5-active site as defined by the atomic coordinates of S-adenosyl methionine (SAM) and amino acids Leu319, Phe327, Glu435, Leu437, Glu444, and Phe580, according to Table A, wherein the compound has a biochemical IC50 for PRMT5 of less than 100 nM. In certain embodiments, the PRMT5 inhibitor is capable of undergoing a pi-cation interaction with SAM. In certain embodiments, the PRMT5 inhibitor is capable of undergoing a pi-stacking interaction with Phe327. In certain embodiments, the PRMT5 inhibitor is capable of interacting with Glu444.

Further provided herein are computer-assisted methods for identifying potential PRMT5 binding compounds, using a programmed computer comprising a processor, a data storage system, an input device, and an output device, wherein the methods include a) inputting into the programmed computer through said input device data comprising the atomic coordinates of a subset of the atoms generated from a complex of PRMT5 and a binding compound, thereby generating a criteria data set; b) comparing, using said processor, said criteria data set to a computer database of chemical structures stored in said computer data storage system; c) selecting from said database, using computer methods, chemical structures having a portion that is structurally similar to said criteria data set; and d) outputting to said output device the selected chemical structures having a portion similar to said criteria data set. The subsets of atomic coordinates used in such methods may include one or more of amino acids Leu319, Phe327, Glu435, Leu437, Glu444, and Phe580, and optionally one or more of amino acids Leu312, Thr323, Tyr324, Glu328, Lys333, Tyr334, Gly438, Ser439, Val503, Ser578, Trp579, and Pro581 as set forth in Table A.

Further provided herein are computer readable media that include the atomic coordinates of PRMT5 as set forth in Table A and may optionally further include programming for displaying a molecular model of PRMT5, programming for identifying a binding compound to PRMT5, and/or a database of structures of drug candidates. Further provided herein are computer systems that include such computer-readable media.

Further provided herein are computer systems that include a memory unit comprising atomic coordinates defining PRMT5 as set forth in Table A; and a processor in electrical communication with the memory unit; wherein the processor generates a molecular model having a three dimensional structure representative of at least a portion of PRMT5.

Further provided herein are computer systems that include a memory unit comprising atomic coordinates of amino acids Leu319, Phe327, Glu435, Leu437, Glu444, and Phe580, of PRMT5 as set forth in Table A; and a processor in electrical communication with the memory unit; wherein the processor generates a molecular model having a three dimensional structure representative of at least a portion of PRMT5. The memory unit may optionally further comprise the atomic coordinates of one or more of amino acids Leu312, Thr323, Tyr324, Glu328, Lys333, Tyr334, Gly438, Ser439, Val503, Ser578, Trp579, and Pro581 as set forth in Table A.

In certain embodiments, the present disclosure provides a computer readable medium comprising the atomic coordinates of the complex PRMT5-Compound A6 as set forth in Table A1.

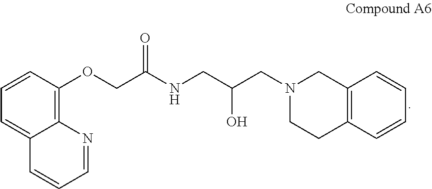 [SMILES: O=C(COc1cccc2cccnc12)NCC(O)CN1CCc2ccccc2C1]

In certain embodiments, the present disclosure provides compounds or pharmaceutical compositions to treat a proliferative disorder, cancer metabolic disorder, diabetes, obesity, blood disorder, hemoglobinopathies, sickle cell anemia, or β-thalessemia. In certain embodiments, the present disclosure provides compounds or pharmaceutical compositions to treat hematological cancers, lung cancer, prostate cancer, melanoma, or pancreatic cancer.

### Compounds

As generally described above, provided herein are compounds useful as PRMT5 inhibitors. In some embodiments, the present disclosure provides a compound of Formula (I):

Ring AA-M-Ring BB I

wherein:

Ring AA is an optionally substituted aryl moiety;

Ring BB is an optionally substituted aryl or heteroaryl moiety, wherein the aryl or heteroaryl moiety is capable of forming a cation-pi interaction with S-adenosyl methionine (SAM);

M is an acyclic linker moiety 3-10 atoms in length, which allows for the planes of Ring A and Ring B to be between 75° and 105° relative to each other, and includes a carbonyl group, wherein Ring A is attached directly to the carbonyl group, or to the alpha-carbon of the carbonyl group; and

wherein the compound has a biochemical IC₅₀ for PRMT5 of less than 100 nM.

In certain embodiments, Ring AA is a monocyclic or bicyclic, saturated, partially unsaturated, or aromatic ring having 0, 1, 2, 3, 4, or 5 heteroatoms independently selected from nitrogen, oxygen, and sulfur, wherein Ring AA is substituted with 0, 1, 2, 3, 4, or 5 R^{y} groups. In certain embodiments, Ring AA is a monocyclic aryl moiety. In certain embodiments, Ring AA is an optionally substituted, fused bicyclic heteroaryl moiety. In certain embodiments, Ring AA is an unsubstituted, fused bicyclic heteroaryl moiety. In certain embodiments, Ring AA is a phenyl moiety fused to a heterocyclic moiety. In certain embodiments, Ring AA is a phenyl moiety fused to a heteroaryl moiety. In certain embodiments, Ring AA is a phenyl moiety fused to a 5- or 6-membered heteroaryl moiety.

In certain embodiments, Ring AA is a phenyl moiety fused to a 5- or 6-membered heteroaryl moiety with one nitrogen. In certain embodiments, Ring AA is a phenyl moiety fused to a 5- or 6-membered heteroaryl moiety with two nitrogen.

In certain embodiments, Ring AA is of formula (Ia-1):

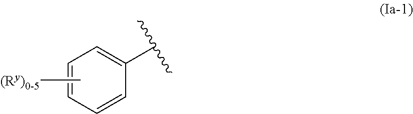

wherein

R^{y} is described herein.

In certain embodiments, Ring AA is of formula (Ia-2):

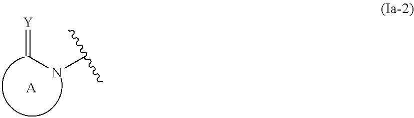

wherein:

Ring A is an optionally substituted, 5- to 14-membered, monocyclic or bicyclic, heterocyclyl or heteroaryl having 1-4 heteroatoms independently selected from nitrogen, oxygen, and sulfur; and

Y is O or S.

In certain embodiments, Ring AA is of formula (Ia-3):

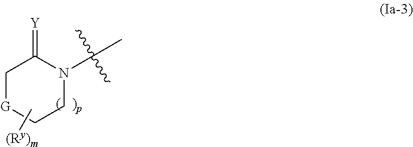

wherein:

Y is O or S;

G is NR^{2C}, CR³R^{4C}, O or S;

R^{2C} is selected from the group consisting of optionally substituted aliphatic, optionally substituted carbocyclyl, optionally substituted aryl, optionally substituted heterocyclyl, optionally substituted heteroaryl, —C(O)R^{A}, —C(O)OR^{A}, —C(O)SR^{A}, —C(O)N(R^{B})₂, —C(═NR^{B})R^{A}, —C(═NR^{B})N(R^{B})₂, —C(═S)R^{A}, —C(═S)N(R^{B})₂, —S(═O)R^{A}, —SO₂R^{A}, and —SO₂N(R^{B})₂;

R³ is selected from the group consisting of hydrogen, halo, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(O)R^{A}, —C(O)OR^{A}, —C(O)SR^{A}, —C(O)N(R^{B})₂, —OC(O)R^{A}, —NR^{B}C(═O)R^{A}, —NR^{B}C(═O)N(R^{B})₂, —SC(═O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —SO₂R^{A}, —NR^{B}SO₂R^{A}, and —SO₂N(R^{B})₂;

each R^{A} is independently selected from the group consisting of hydrogen, optionally substituted aliphatic, optionally substituted carbocyclyl, optionally substituted heterocyclyl, optionally substituted aryl, and optionally substituted heteroaryl;

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring;

R^{4C} is selected from the group consisting of hydrogen, halo, or optionally substituted aliphatic;

each R^{y} is independently selected from the group consisting of halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, C(═NR^{B})R^{A}, C(NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂, or two adjacent R^{y} groups may be taken together with their intervening atoms to form a saturated, partially unsaturated, or aromatic ring having 0-4 heteroatoms selected from nitrogen, oxygen, and sulfur;

p is 0, 1, or 2; and

m is 0, 1, 2, 3, or 4.

In certain embodiments, Ring AA is of formula (Ia-3):

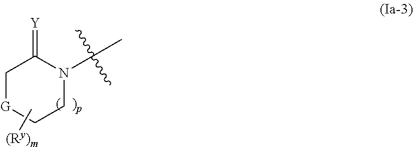

wherein:

Y, N, R^{y}, m and p are as described herein;

R^{2C} is selected from the group consisting of:

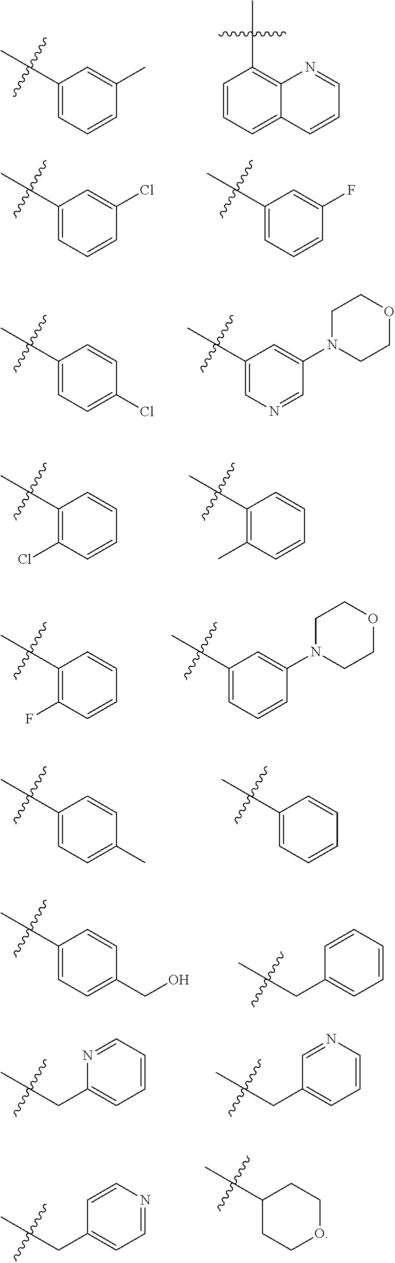

In certain embodiments, Ring AA is of formula (Ia-4):

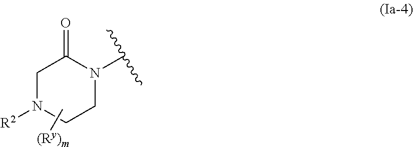

wherein:

R² is selected from the group consisting of optionally substituted aliphatic, optionally substituted carbocyclyl, optionally substituted aryl, optionally substituted heterocyclyl, optionally substituted heteroaryl, —C(O)R^{A}, —C(O)OR^{A}, —C(O)SR^{A}, —C(O)N(R^{B})₂, —C(═NR^{B})R^{A}, —C(═NR^{B})N(R^{B})₂, —C(═S)R^{A}, —C(═S)N(R^{B})₂, —S(═O)R^{A}, —SO₂R^{A}, and —SO₂N(R^{B})₂;

each R^{y} is independently selected from the group consisting of halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR_{B}SO₂R^{A}, and —SO₂N(R^{B})₂, or two adjacent R^{y} groups may be taken together with their intervening atoms to form a saturated, partially unsaturated, or aromatic ring having 0-4 heteroatoms selected from nitrogen, oxygen, and sulfur;

each R^{A} is independently selected from the group consisting of hydrogen, optionally substituted aliphatic, optionally substituted carbocyclyl, optionally substituted heterocyclyl, optionally substituted aryl, and optionally substituted heteroaryl;

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring;

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring;

m is 0, 1, 2, 3, or 4.

In certain embodiments, Ring AA is of formula (Ia-5):

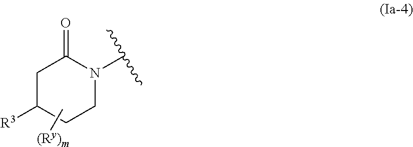

wherein:

R³ is selected from the group consisting of hydrogen, halo, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(O)R^{A}, —C(O)OR^{A}, —C(O)SR^{A}, —C(O)N(R^{B})₂, —OC(O)R^{A}, —NR^{B}C(═O)R^{A}, —NR^{B}C(═O)N(R^{B})₂, —SC(═O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —SO₂R^{A}, —NR^{B}SO₂R^{A}, and —SO₂N(R^{B})₂;

each R^{y} is independently selected from the group consisting of halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂, or two adjacent R^{y} groups may be taken together with their intervening atoms to form a saturated, partially unsaturated, or aromatic ring having 0-4 heteroatoms selected from nitrogen, oxygen, and sulfur;

each R^{A} is independently selected from the group consisting of hydrogen, optionally substituted aliphatic, optionally substituted carbocyclyl, optionally substituted heterocyclyl, optionally substituted aryl, and optionally substituted heteroaryl;

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring;

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring; and

m is 0, 1, 2, 3, or 4.

In certain embodiments, Ring AA is of formula (Ia-6):

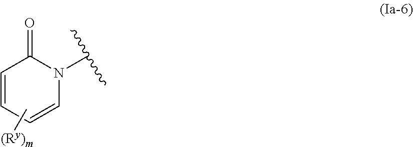

wherein R^{y} and m are described herein.

In certain embodiments, Ring AA is of formula (Ia-7):

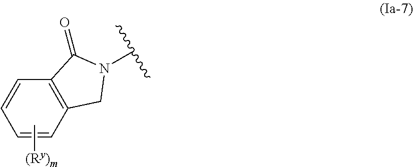

wherein R^{y} and m are described herein.

In certain embodiments, Ring AA is of formula (Ia-8):

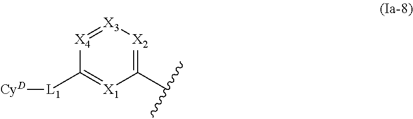

wherein:

L₁ is a bond, —O—, —S—, —N(R)—, —C(O)—, —C(O)N(R)—, —N(R)C(O)N(R)—, —N(R)C(O)—, —N(R)C(O)O—, —OC(O)N(R)—, —SO₂—, —SO₂N(R)—, —N(R)SO₂—, —OC(O)—, —C(O)O—, or an optionally substituted, straight or branched, C₁₋₆ aliphatic chain wherein one, two, or three methylene units of L₁ are optionally and independently replaced by —O—, —S—, —N(R)—, —C(O)—, —C(O)N(R)—, —N(R)C(O)N(R)—, —N(R)C(O)—, —N(R)C(O)O—, —OC(O)N(R)—, —SO₂—, —SO₂N(R)—, —N(R)SO₂—, —OC(O)—, or —C(O)O—;

Cy^{D} is an optionally substituted, monocyclic, bicyclic or tricyclic, saturated, partially unsaturated, or aromatic ring having 0-4 heteroatoms independently selected from nitrogen, oxygen, and sulfur;

X₁, X₂, X₃, and X₄ are independently selected from the group consisting of N, CH, and CR^{y}, provided that at least one of X₂, X₃, and X₄ is not N;

each R^{y} is independently selected from the group consisting of halo, —CN, —NO₂, optionally substituted aliphatic, optionally substituted carbocyclyl; optionally substituted phenyl, optionally substituted heterocyclyl, optionally substituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —OC(═O)R^{A}, —NR^{B}C(═O)R^{A}, —NR^{B}C(═O)N(R^{B})₂, —SC(═O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —SO₂R^{A}, —NR^{B}SO₂R^{A}, and —SO₂N(R^{B})₂;

each R^{A} is independently selected from the group consisting of hydrogen, optionally substituted aliphatic, optionally substituted carbocyclyl, optionally substituted heterocyclyl, optionally substituted aryl, and optionally substituted heteroaryl; and

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring.

In certain embodiments, Ring AA is of formula (Ia-9):

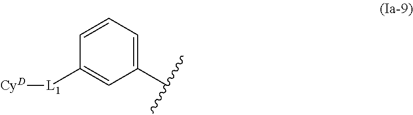

wherein L_{D}, L₁ are as defined herein.

In certain embodiments, Ring AA is of formula (Ia-10):

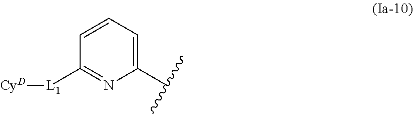

wherein L_{D}, L₁ are as defined herein.

In certain embodiments, Ring AA is of formula (Ia-11):

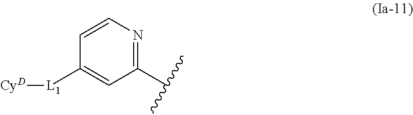

wherein L_{D}, L₁ are as defined herein.

In certain embodiments, Ring AA is of formula (Ia-12):

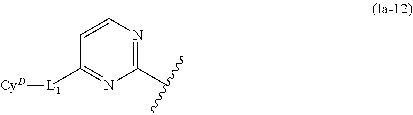

wherein L_{D}, L₁ are as defined herein.

In certain embodiments, Ring AA is of formula (Ia-13):

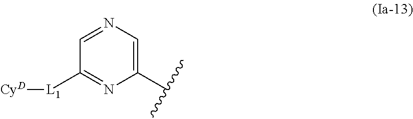

wherein L_{D}, L₁ are as defined herein.

In certain embodiments, Ring AA is of formula (Ia-14):

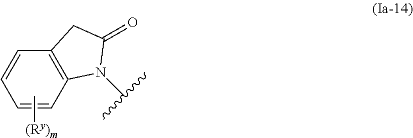

wherein R^{y} and m are described herein.

In certain embodiments, Ring AA is of formula (Ia-15):

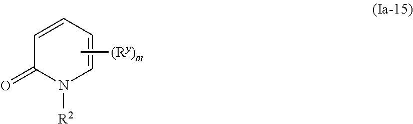

wherein R¹, R^{y} and m are described herein.

In certain embodiments, Ring AA is of formula (Ia-16):

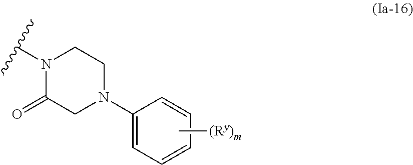

wherein R^{y} and m are described herein.

In certain embodiments, Ring AA is of formula (Ia-17):

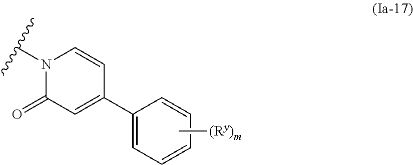

wherein R^{y} and m are described herein.

In certain embodiments, Ring AA is of formula (Ia-18):

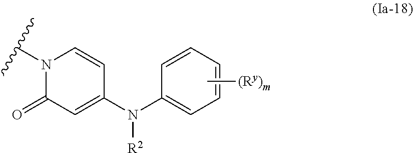

wherein R², R^{y} and m are described herein.

In certain embodiments, Ring AA is of formula (Ia-19):

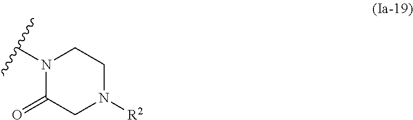

wherein R², R^{y} and m are described herein.

In certain embodiments, Ring AA is of formula (Ia-20):

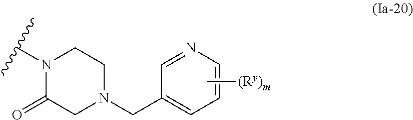

wherein R^{y} and m are described herein.

In certain embodiments, Ring AA is of formula (Ia-21):

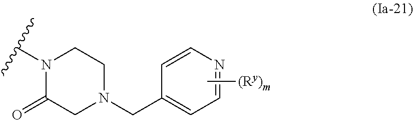

wherein R^{y} and m are described herein.

In certain embodiments, Ring AA is of formula (Ia-22):

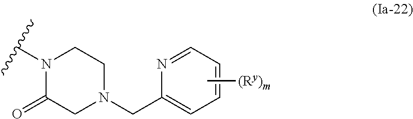

wherein R^{y} and m are described herein.

In certain embodiments, Ring AA is of formula (Ia-22):

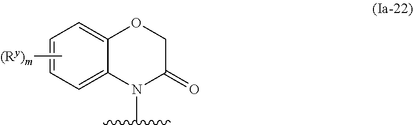

wherein R^{y} and m are described herein.

In certain embodiments, Ring AA is of formula (Ia-23):

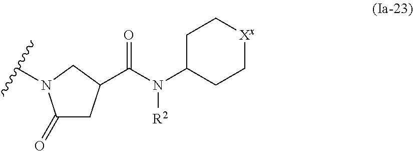

wherein X^{x} is NR², O, or S; and R², R^{y} and m each are independently described herein.

In certain embodiments, Ring AA is selected from the group consisting of:

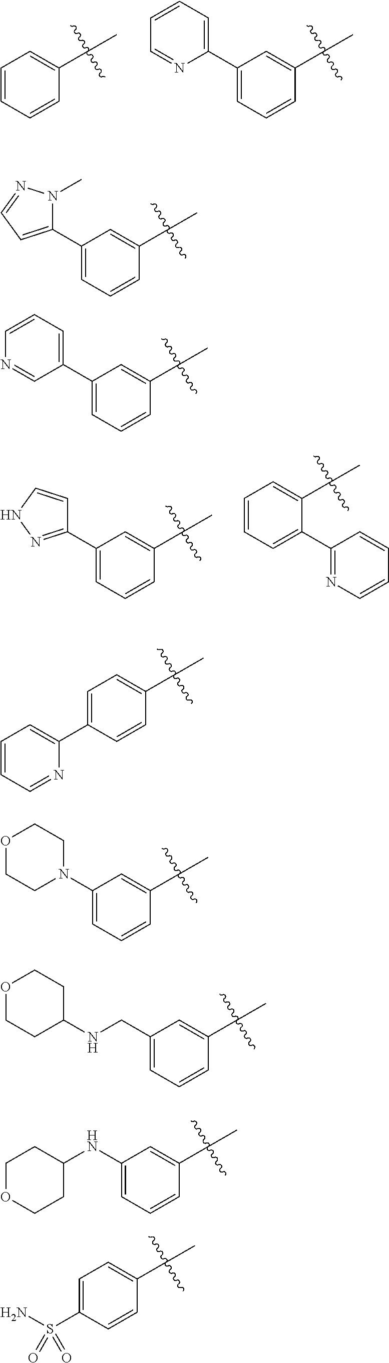

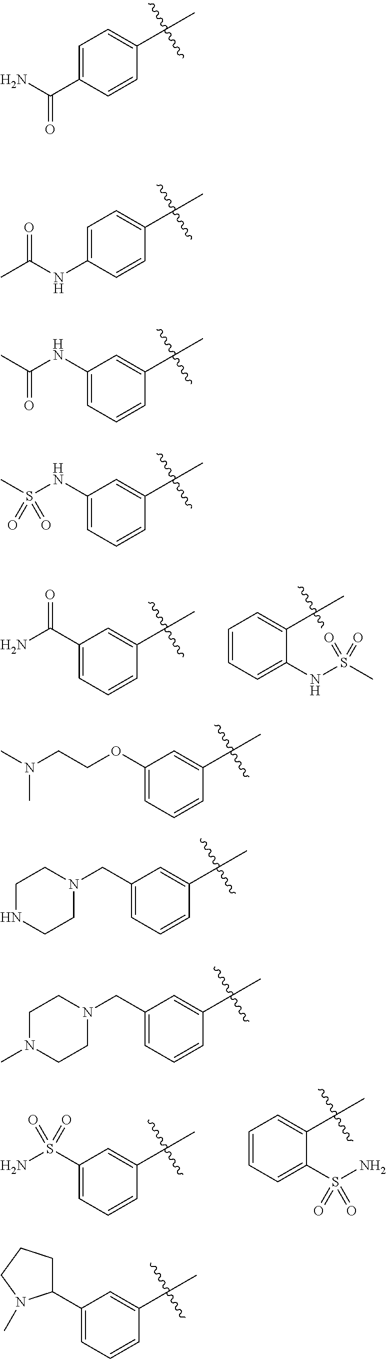

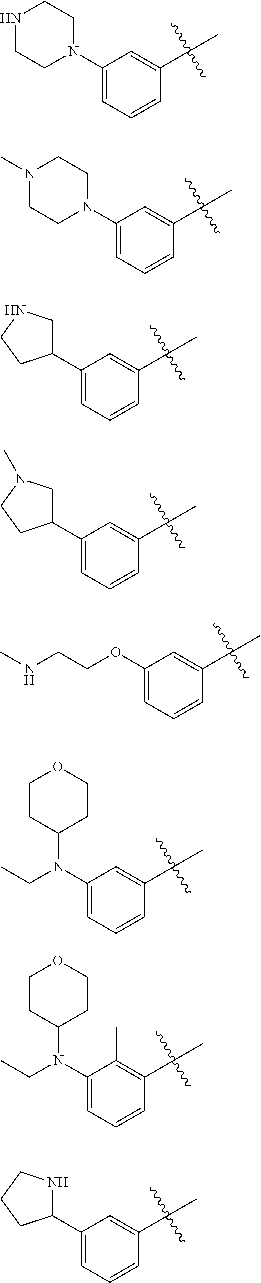

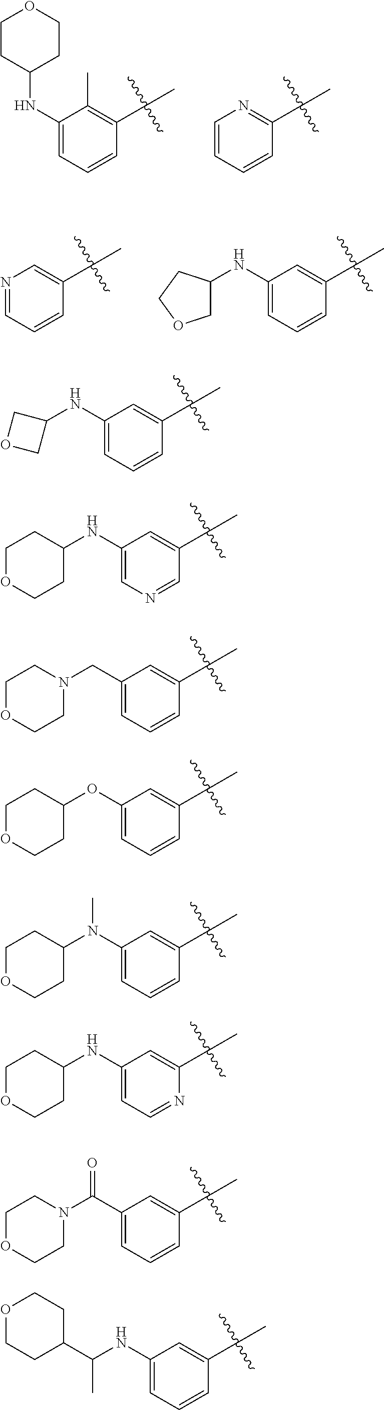

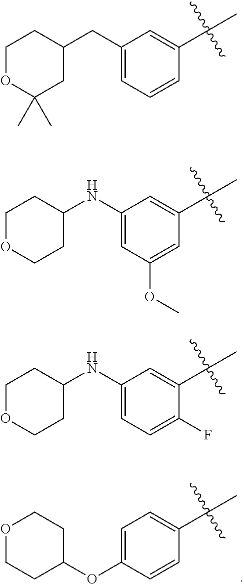 [SMILES: *c1cccc(CC2CCOC(C)(C)C2)c1 ; *c1cc(NC2CCOCC2)cc(OC)c1 ; *c1cc(NC2CCOCC2)ccc1F ; *c1ccc(OC2CCOCC2)cc1]

In certain embodiments, Ring BB is an optionally substituted, bicyclic heteroaryl moiety. In certain embodiments, Ring BB is an optionally substituted, bicyclic heteroaryl moiety with 1-4 nitrogen atoms. In certain embodiments, Ring BB is an unsubstituted bicyclic heteroaryl moiety. In certain embodiments, Ring BB is optionally substituted bihydroisoquinoline. In certain embodiments, Ring BB is optionally substituted tetrahydroisoquinoline. In certain embodiments, Ring BB is unsubstituted tetrahydroisoquinoline. In certain embodiments, Ring BB is optionally substituted isoindoline. In certain embodiments, Ring BB is unsubstituted isoindoline. In certain embodiments, Ring BB is an optionally substituted amino-aryl moiety. In certain embodiments, Ring BB is optionally substituted benzylamine. In certain embodiments, Ring BB is unsubstituted benzylamine.

In certain embodiments, Ring BB is of formula (Ib-1)

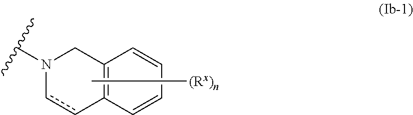

wherein: [symbol]
represents a single or double bond;

R^{x} is independently selected from the group consisting of halo, —CN, optionally substituted aliphatic, and —OR;

R′ is hydrogen or optionally substituted aliphatic; and

n is 0, 1, 2, 3, 4, 5, 6, 7, or 8.

In certain embodiments, Ring BB is of formula (Ib-2)

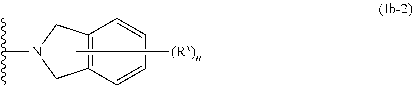

wherein:

R^{x} is independently selected from the group consisting of halo, —CN, optionally substituted aliphatic, and —OR;

R′ is hydrogen or optionally substituted aliphatic; and

n is 0, 1, 2, 3, 4, 5, 6, 7, or 8.

In certain embodiments, M is with the 4-8 atoms in length. In certain embodiments, M is a linker 4 atoms in length. In certain embodiments, M is a linker 5 atoms in length. In certain embodiments, the atoms of M are selected from the group consisting of C, N, O, and S. In certain embodiments, the atoms of M are selected from the group consisting of C, N, and O. In certain embodiments, M comprises an amide moiety. In certain embodiments, M comprises a hydroxyl moiety. In certain embodiments, M comprises a sulfonamide moiety. In certain embodiments, M comprises an ester moiety. In certain embodiments, M provides a distance between Ring A and Ring B ranging from approximately 6 Angstroms to approximately 10 Angstroms. In certain embodiments, M provides a distance between Ring A and Ring B ranging from approximately 8 Angstroms to approximately 9 Angstroms. In certain embodiments, M allows for the planes of Ring A and Ring B to be at an angle ranging from 85° to approximately 95°.

In certain embodiments, M is of formula (Ic):

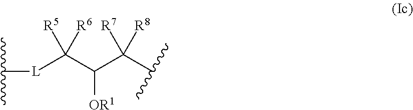

wherein:

R¹ is hydrogen, R^{z}, or —C(O)R^{z}, wherein R^{z} is optionally substituted C₁₋₆ alkyl;

L is a bond, —N(R)C(O)—, —C(O)N(R)—, —N(R)C(O)N(R)—, —N(R)C(O)O—, —OC(O)N(R)—, —O—, —N(R)—, —C(R²)(R³)—, —O—CR²R³, —N(R)—CR²R³—, —O—CR²R³—O—, —N(R)—CR²R³—O, —N(R)—CR²R³—N(R)—, —O—CR²R³—N(R)—, —CR²R³—O—, —CR²R³—N(R)—, —O—CR²R³—CR⁹R¹⁰—, —N(R)—CR²R³—CR⁹R¹⁰—, —CR²R³—CR⁹R¹⁰—O—, —CR²R³—CR⁹R¹⁰—N(R)—, or —CR²R³—CR⁹R¹⁰—;

R⁵, R⁶, R⁷, and R⁸ are independently hydrogen, halo, or optionally substituted aliphatic; and

R⁹ and R¹⁰ are independently selected from the group consisting of hydrogen, halo, —CN, —NO₂, optionally substituted aliphatic, optionally substituted carbocyclyl; optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂; or R⁹ and R¹⁰ are taken together with their intervening atoms to form an optionally substituted carbocyclic or heterocyclic ring.

In certain embodiments, M is of formula (Id):

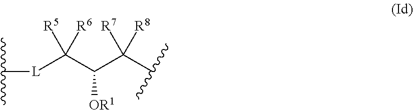

wherein R¹, R⁵, R⁶, R⁷ and R⁸ are as described herein.

In certain embodiments, M is of formula (Ie):

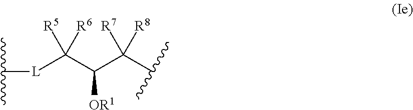

wherein R¹, R⁵, R⁶, R⁷ and R⁸ are as described herein.

In certain embodiments, M is of formula (If):

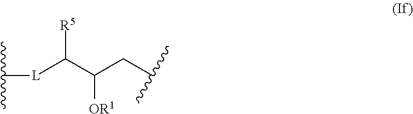

wherein R¹ as described herein.

In certain embodiments, M is of formula (Ic-1):

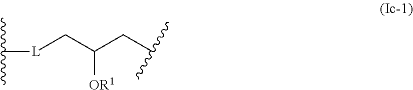

wherein R¹ as described herein.

In certain embodiments, M is of formula (Ic-2):

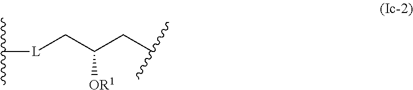

wherein R¹ as described herein.

In certain embodiments, M is of formula (Ic-3):

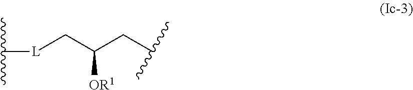

wherein R¹ as described herein.

In certain embodiments, M is of formula (Ic-4).

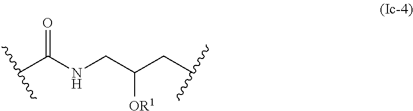

wherein R¹ as described herein.

In certain embodiments, M is of formula (Ic-5).

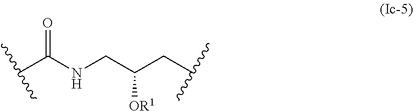

wherein R¹ as described herein.

In certain embodiments, M is of formula (Ic-6).

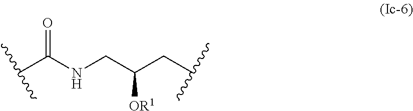

wherein R¹ as described herein.

In certain embodiments, M is of formula (Ic-7).

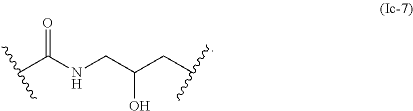 [SMILES: *CC(O)CNC(*)=O]

In certain embodiments, M is of formula (Ic-8).

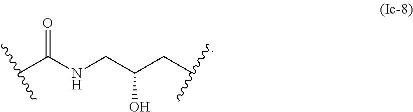 [SMILES: *C[C@H](O)CNC(*)=O]

In certain embodiments, M is of formula (Ic-9).

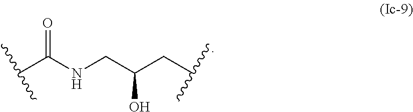 [SMILES: *C[C@@H](O)CNC(*)=O]

In certain embodiments, M is of formula (Ic-10):

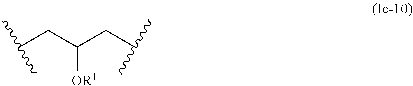

wherein R¹ as described herein.

In certain embodiments, M is of formula (Ic-11):

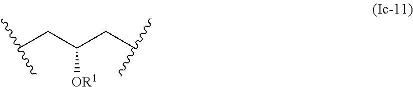

wherein R¹ as described herein.

In certain embodiments, M is of formula (Ic-12):

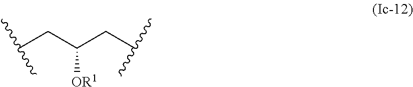

wherein R¹ as described herein.

In certain embodiments, M is of formula (Ie):

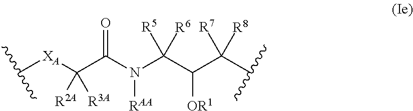

wherein:

R¹ and R^{AA} are each independently hydrogen, R^{z}, or —C(O)R^{z}, wherein R^{z} is optionally substituted C₁₋₆ alkyl;

X_{A} is a bond, —O—, —N(R)—, —CR^{4A}R^{5A}—, —O—CR^{4A}R^{5A}, —N(R)—CR^{4A}R^{5A}—, —O—CR^{4A}R^{5A}—O—, —N(R)—CR^{4A}R^{5A}—O, —N(R)—CR^{4A}R^{5A}—N(R)—, —O—CR^{4A}R^{5A}—N(R)—, —CR^{4A}R^{5A}—O—, —CR^{4A}R^{5A}—N(R)—, —O—CR^{4A}R^{5A}—CR^{6A}R^{7A}—, —N(R)—CR^{4A}R^{5A}—CR^{6A}R^{7A}—, —CR^{6A}R^{7A}—CR^{4A}R^{5A}—O—, —CR^{6A}R^{7A}—CR^{4A}R^{5A}—N(R)—, or —CR^{6A}R^{7A}—CR^{4A}R^{5A}—;

R^{2A} and R^{3A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂; or R^{2A} and R^{3A} are taken together with their intervening atoms to form an optionally substituted carbocyclic or heterocyclic ring;

R^{4A} and R^{5A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂; or R^{4A} and R^{5A} are taken together with their intervening atoms to form an optionally substituted carbocyclic or heterocyclic ring;

R^{6A} and R^{7A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂; or R^{6A} and R^{7A} are taken together with their intervening atoms to form an optionally substituted carbocyclic or heterocyclic ring;

each R^{A} is independently selected from the group consisting of hydrogen, optionally substituted aliphatic, optionally substituted carbocyclyl, optionally substituted heterocyclyl, optionally substituted aryl, and optionally substituted heteroaryl;

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring.

In certain embodiments, M is of formula (If):

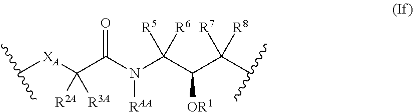

wherein X_{A}, R^{2A}, R^{3A}, R^{AA}, R¹, R⁵, R⁶, R⁷ and R⁸ are as described herein.

In certain embodiments, M is of formula (Ig):

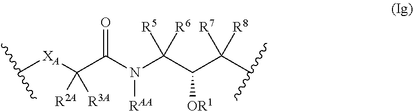

wherein X_{A}, R^{2A}, R^{3A}, R^{AA}, R¹, R⁵, R⁶, R⁷ and R⁸ are as described herein.

In certain embodiments, M is of formula (Ie-1):

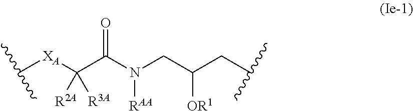

wherein X_{A}, R^{2A} and R¹ are as described herein.

In certain embodiments, M is of formula (Ie-2):

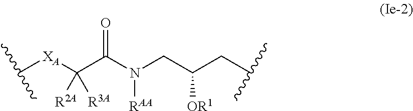

wherein X_{A}, R^{2A}, R^{3A}, R^{AA}, and R¹ are as described herein.

In certain embodiments, M is of formula (Ie-3):

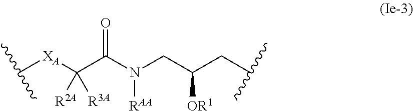

wherein X_{A}, R^{2A}, R^{3A}, R^{AA}, and R¹ are as described herein.

In certain embodiments, M is of formula (Ie-I):

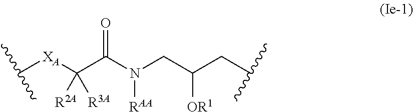

wherein X_{A} is C, O or N, R^{2A}, R^{3A}, R^{AA}, and R¹ are as described herein.

In certain embodiments, M is of formula (Ie-2):

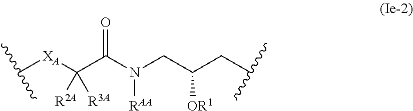

wherein X_{A} is C, O or N, R^{2A}, R^{3A}, R^{AA}, and R¹ are as described herein.

In certain embodiments, M is of formula (Ie-3):

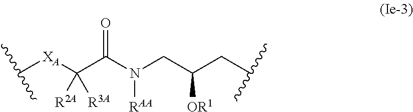

wherein X_{A} is C, O or N, R^{2A}, R^{3A}, R^{AA}, and R¹ are as described herein.

In certain embodiments, M is of formula (Ie-1):

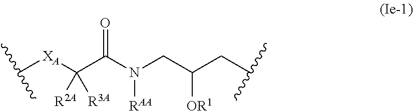

wherein X_{A} is C, O or N, R^{AA} and R¹ are H, R^{2A}, and R^{3A} are as described herein.

In certain embodiments, M is of formula (Ie-2):

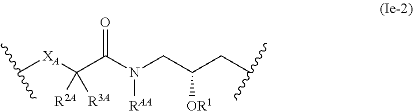

wherein X_{A} is C, O or N, R^{AA} and R¹ are H, R^{2A}, and R^{3A} are as described herein.

In certain embodiments, M is of formula (Ie-3):

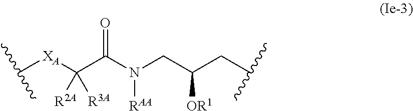

wherein X_{A} is C, O or N, R^{AA} and R¹ are H, R^{2A}, and R^{3A} are as described herein.

In certain embodiments, a provided compound is of Formula (II):

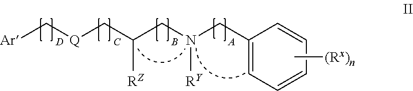

or a pharmaceutically acceptable salt thereof,
wherein:

Q is —N(R)C(O)—, —C(O)N(R)—, —N(R)C(O)N(R)—, —N(R)C(O)O—, SO₂NR—, or —OC(O)N(R)—;

each R is independently hydrogen, nitrogen protecting group or optionally substituted C₁₋₆ aliphatic;

Ar′ is a monocyclic or bicyclic aromatic ring having 0-4 heteroatoms independently selected from nitrogen, oxygen, and sulfur, wherein Ar′ is substituted with 0, 1, 2, 3, 4, or 5 R^{x} groups, as valency permits;

each R^{x} is independently selected from the group consisting of halo, —CN, optionally substituted aliphatic, —OR′, and —N(R″)₂;

R^{Y} is hydrogen, nitrogen protecting group or optionally substituted aliphatic;

R^{z} is hydrogen or hydroxyl group;

R′ and R″ are independently hydrogen, nitrogen or oxygen protecting group, optionally substituted C₁₋₆ aliphatic;

A, B, C, and D are independently 0, 1, or 2;

n is 0, 1, 2, 3, 4, or 5; and

the nitrogen substituted by R^{Y} optionally forms a ring system with the carbon linked with R^{z}, or forms a ring system with the phenyl group substituted by (R^{x}) n.

In certain embodiments, when Ring AA is of formula (Ia-1);

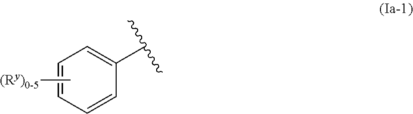

Ring BB is of formula (Ib-1) or formula (Ib-2):

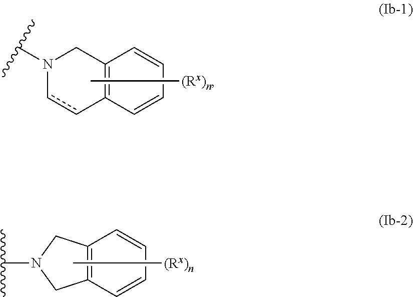

and M is of formula (Ie) or formula (If):

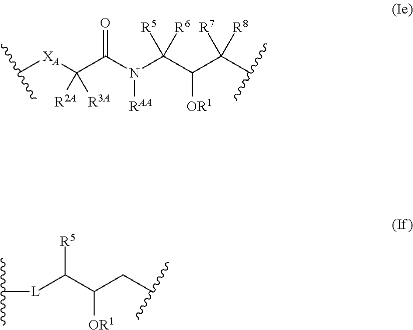

wherein R^{x}, R^{y}, R¹, R⁵, R⁷, R⁸, R^{2A}, R^{3A}, R^{AA}, X_{A}, G, Y are described herein.

In certain embodiments, when Ring AA is of formula (Ia-4):

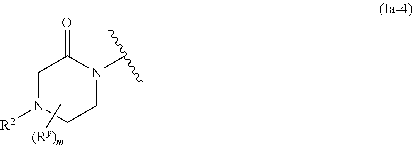

Ring BB is of formula (Ib-1) or formula (Ib-2):

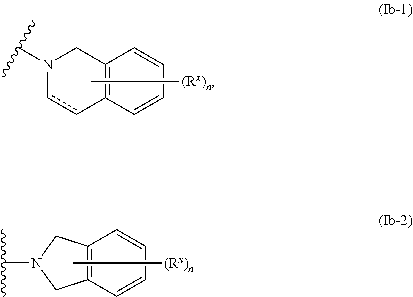

and M is of formula (Ie) or formula (If):

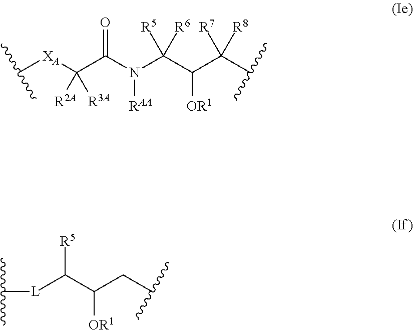

wherein R^{x}, R^{y}, R¹, R⁵, R⁷, R⁸, R^{2A}, R^{3A}, R^{AA}, X_{A}, G, Y are independently described herein.

In certain embodiments, when Ring AA is of formula (Ia-7):

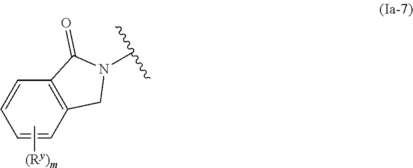

Ring BB is of formula (Ib-1) or formula (Ib-2):

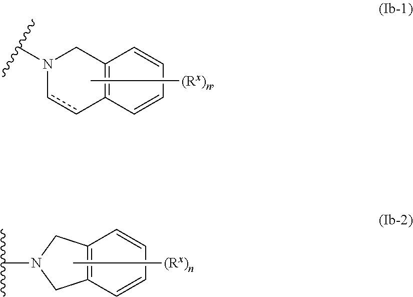

and M is of formula (Ie) or formula (If):

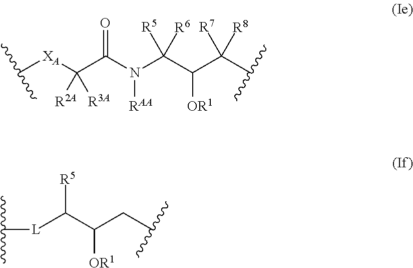

wherein R^{x}, R^{y}, R¹, R⁵, R⁷, R⁸, R^{2A}, R^{3A}, R^{AA}, X_{A}, m, and n are described herein.

In certain embodiments, when Ring is of formula (Ia-3):

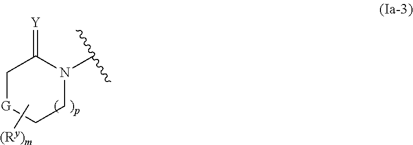

Ring BB is of formula (Ib-1) or formula (Ib-2):

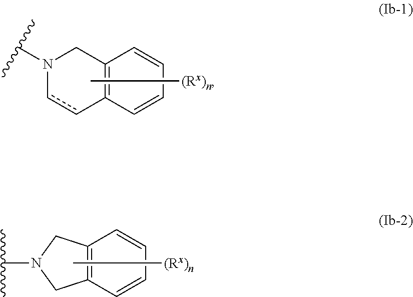

and M is of formula (Ie) or formula (If):

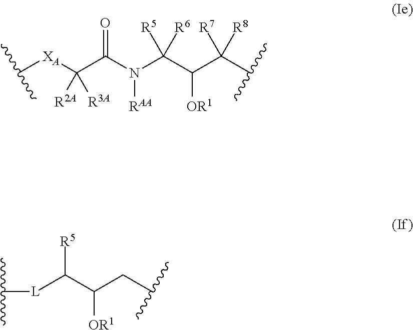

wherein R^{x}, R^{y}, R¹, R⁵, R⁷, R⁸, R^{2A}, R^{3A}, R^{AA}, X_{A}, G, Y are independently described herein.

In certain embodiments, when Ring is of formula (Ia-7):

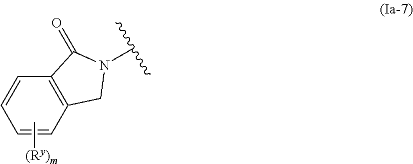

Ring BB is of formula (Ib-1) or formula (Ib-2):

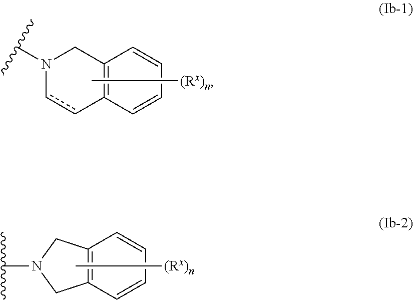

and M is of formula (Ie) or formula (If):

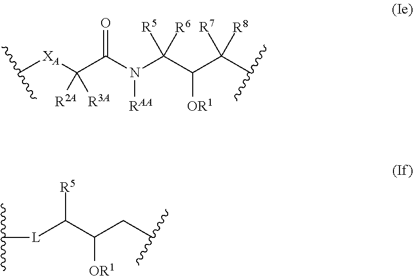

wherein R^{x}, R^{y}, R¹, R⁵, R⁷, R⁸, R^{2A}, R^{3A}, R^{AA}, X_{A}, G, Y are independently described herein.

In certain embodiments, when Ring is of formula (Ia-8):

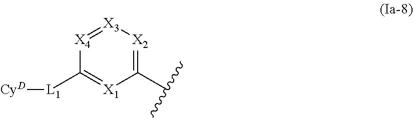

Ring BB is of formula (Ib-1) or formula (Ib-2):

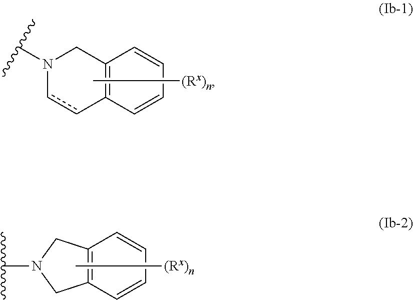

and M is of formula (Ie) or formula (If):

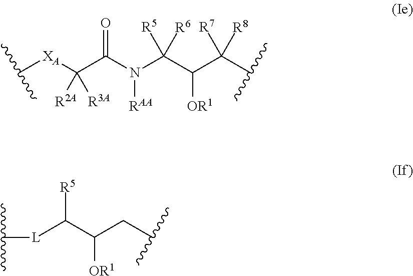

wherein C_{y}^{D}, L₁, X₁, X₂, X₃, X₄, R^{x}, R^{y}, R¹, R⁵, R⁷, R⁸, R^{2A}, R^{3A}, R^{AA}, X_{A}, G, Y are independently described herein.

In certain embodiments, when Ring is of formula (Ia-9):

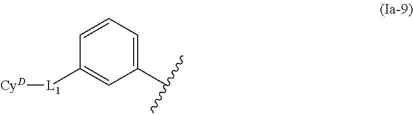

Ring BB is of formula (Ib-1) or formula (Ib-2):

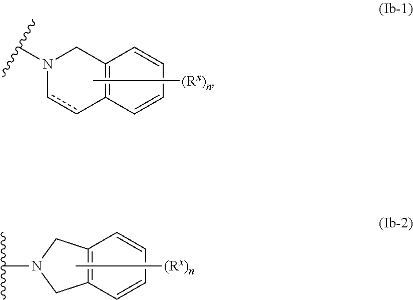

and M is of formula (Ie) or formula (If):

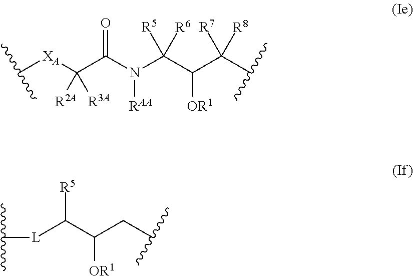

wherein C_{y}^{D}, L₁, X₁, X₂, X₃, X₄, R^{x}, R^{y}, R¹, R⁵, R⁷, R⁸, R^{2A}, R^{3A}, R^{AA}, X_{A}, G, Y are independently described herein.

In certain embodiments, when Ring is of formula (Ia-10):

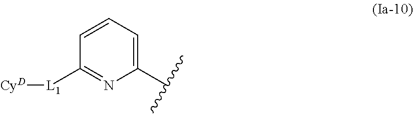

Ring BB is of formula (Ib-1) or formula (Ib-2):

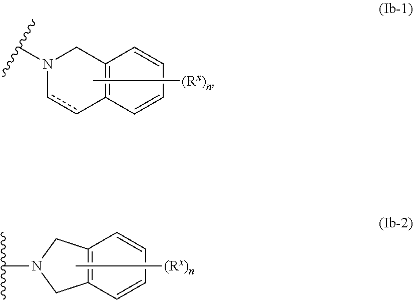

and M is of formula (Ie) or formula (If):

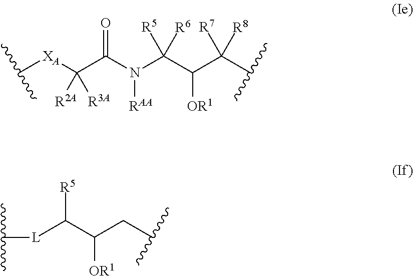

wherein C_{y}^{D}, L₁, X₁, X₂, X₃, X₄, R^{x}, R^{y}, R¹, R⁵, R⁷, R⁸, R^{2A}, R^{3A}, R^{AA}, X_{A}, G, Y are independently described herein.

In certain embodiments, a provided compound is of Formula (III):

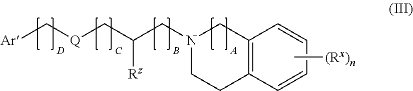

wherein Ar′, Q, R^{z}, R^{x}, A, B, C, and D are described as herein.

In certain embodiments, a provided compound is of Formula (IV):

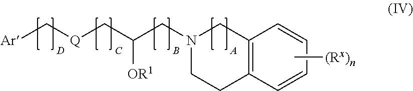

wherein Ar′, Q, R¹, A, B, C, and D are described as herein.

In certain embodiments, a provided compound is of Formula (IV):

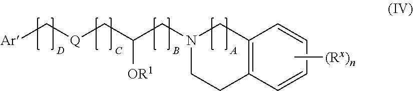

wherein:

Q, R¹, A, B, C, and D are described as herein;

Ar′ is selected from the group consisting of

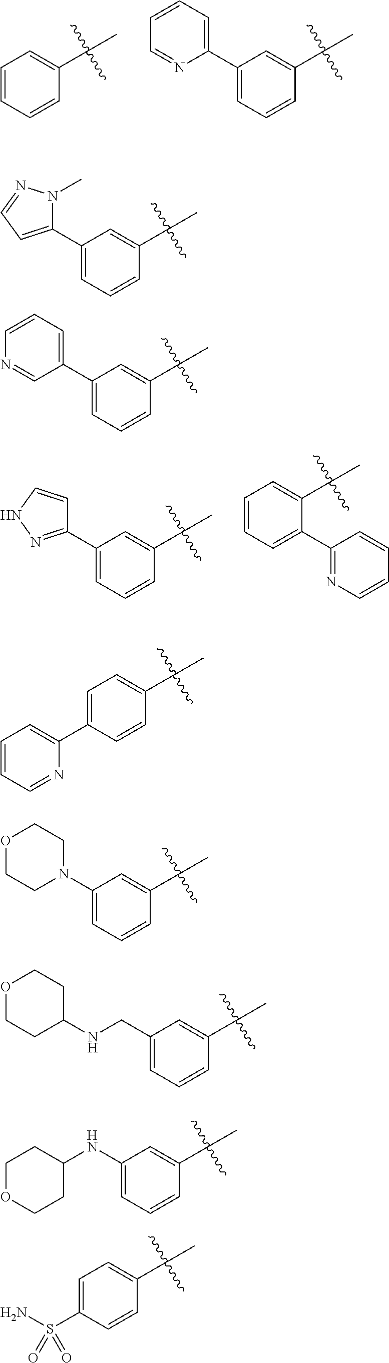

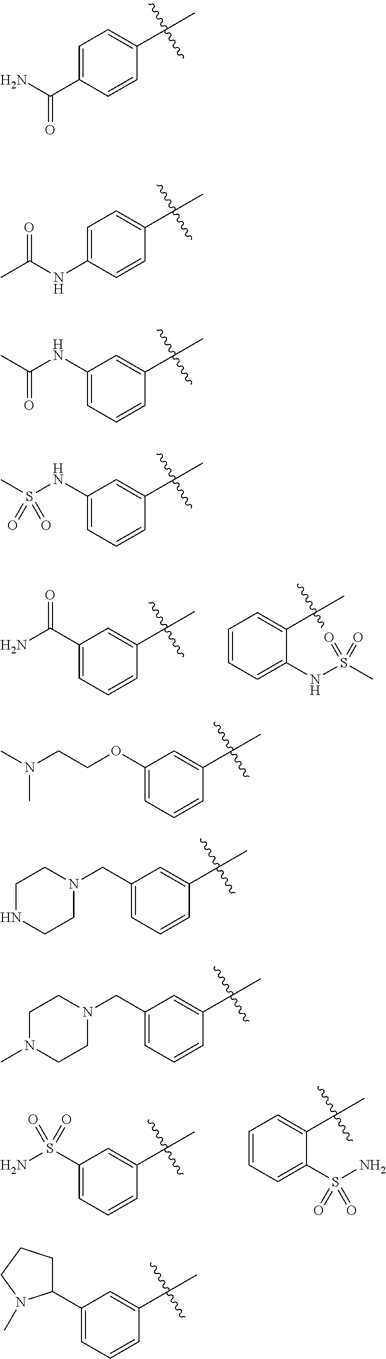

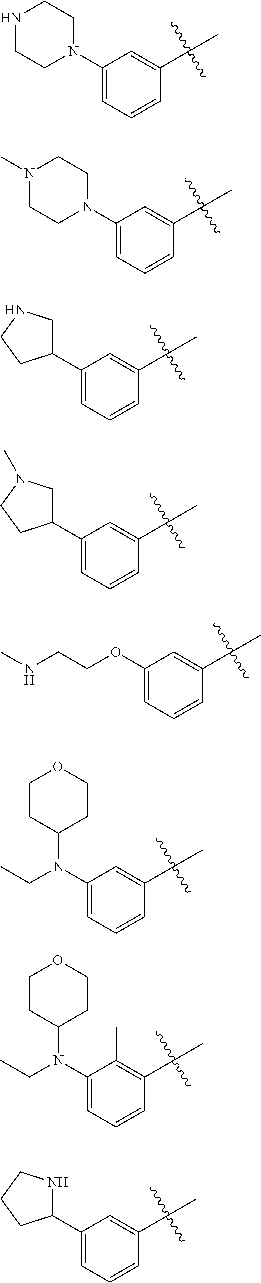

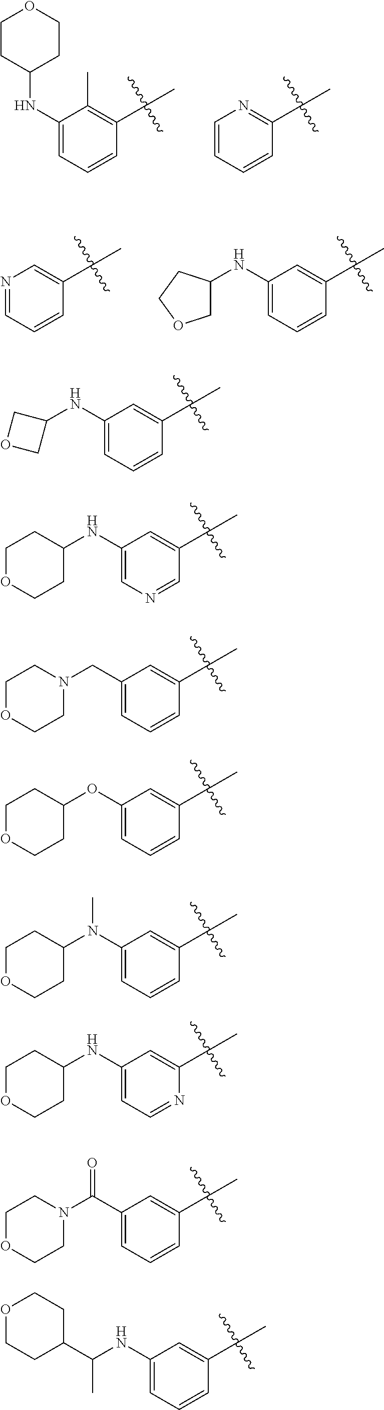

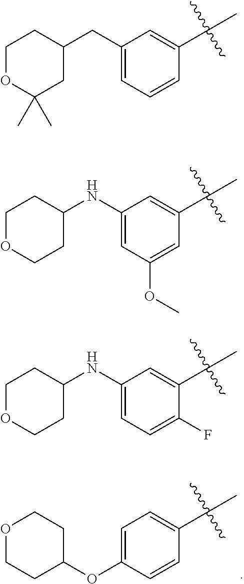 [SMILES: *c1cccc(CC2CCOC(C)(C)C2)c1 ; *c1cc(NC2CCOCC2)cc(OC)c1 ; *c1cc(NC2CCOCC2)ccc1F ; *c1ccc(OC2CCOCC2)cc1]

In certain embodiments, R^{Y} is an optionally substituted aliphatic moiety connected to the ortho position of the phenyl group. In certain embodiments, R^{z} is a hydroxyl group. In certain embodiments, Q is —C(O)N(R)—.

In certain embodiments, a provided compound is a compound listed in Table A-1, or a pharmaceutically acceptable salt thereof

TABLE A-1
Exemplary Compounds

| | | | LC-MS |
|---|---|---|---|
| Cmpd | | Exact | m/z |
| No | Structure | mass | (M + H) |
| A1 | 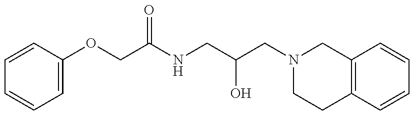 [SMILES: O=C(COc1ccccc1)NCC(O)CN1CCc2ccccc2C1] | 340.1787 | 341.2 |
| A2 | 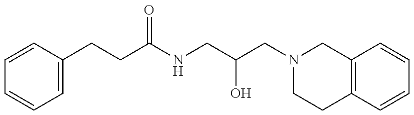 [SMILES: O=C(CCc1ccccc1)NCC(O)CN1CCc2ccccc2C1] | 338.1994 | 339.2 |
| A3 | 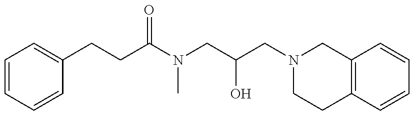 [SMILES: CN(CC(O)CN1CCc2ccccc2C1)C(=O)CCc1ccccc1] | 352.2151 | 353.2 |
| A4 | 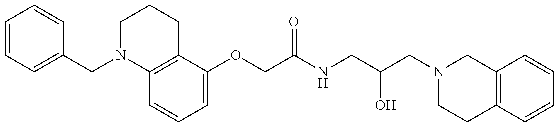 [SMILES: O=C(COc1cccc2c1CCCN2Cc1ccccc1)NCC(O)CN1CCc2ccccc2C1] | 485.2678 | 486.2 |
| A5 | 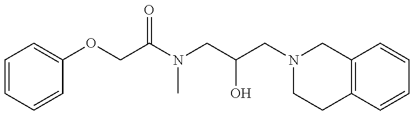 [SMILES: CN(CC(O)CN1CCc2ccccc2C1)C(=O)COc1ccccc1] | 354.1943 | 355.1 |
| A6 | 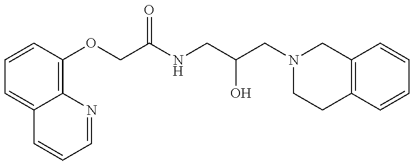 [SMILES: O=C(COc1cccc2cccnc12)NCC(O)CN1CCc2ccccc2C1] | 391.1896 | 392.2 |
| A7 | 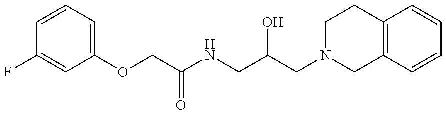 [SMILES: O=C(COc1cccc(F)c1)NCC(O)CN1CCc2ccccc2C1] | 358.1693 | 359.1 |
| A8 | 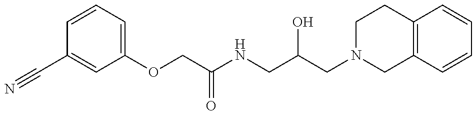 [SMILES: N#Cc1cccc(OCC(=O)NCC(O)CN2CCc3ccccc3C2)c1] | 365.1739 | 366.1 |
| A9 | 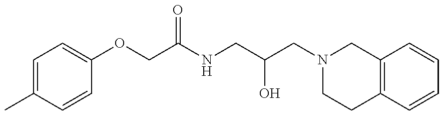 [SMILES: Cc1ccc(OCC(=O)NCC(O)CN2CCc3ccccc3C2)cc1] | 354.1943 | 355.2 |
| A10 | 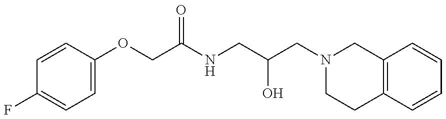 [SMILES: O=C(COc1ccc(F)cc1)NCC(O)CN1CCc2ccccc2C1] | 358.1693 | 359.2 |
| A11 | 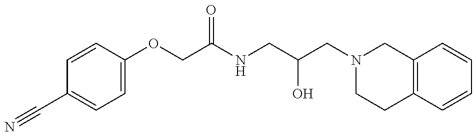 [SMILES: N#Cc1ccc(OCC(=O)NCC(O)CN2CCc3ccccc3C2)cc1] | 365.1739 | 366.2 |
| A12 | 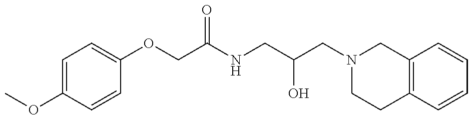 [SMILES: COc1ccc(OCC(=O)NCC(O)CN2CCc3ccccc3C2)cc1] | 370.1893 | 371.2 |
| A13 | 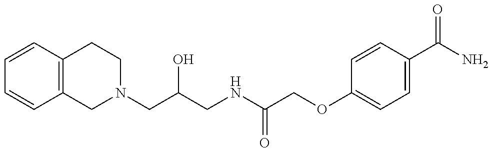 [SMILES: NC(=O)c1ccc(OCC(=O)NCC(O)CN2CCc3ccccc3C2)cc1] | 383.1845 | 384.2 |
| A14 | 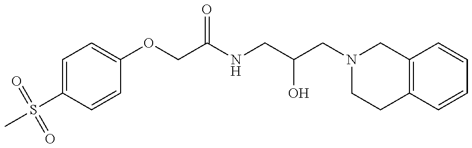 [SMILES: CS(=O)(=O)c1ccc(OCC(=O)NCC(O)CN2CCc3ccccc3C2)cc1] | 418.1562 | 419.2 |
| A15 | 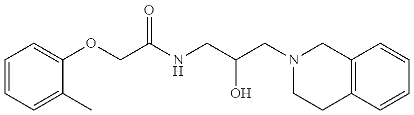 [SMILES: Cc1ccccc1OCC(=O)NCC(O)CN1CCc2ccccc2C1] | 354.1943 | 355.1 |
| A16 | 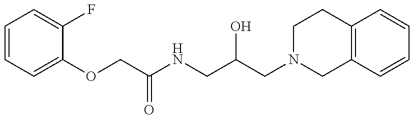 [SMILES: O=C(COc1ccccc1F)NCC(O)CN1CCc2ccccc2C1] | 358.1693 | 359.1 |
| A17 | 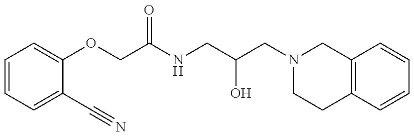 [SMILES: N#Cc1ccccc1OCC(=O)NCC(O)CN1CCc2ccccc2C1] | 365.1739 | 366.1 |
| A18 | 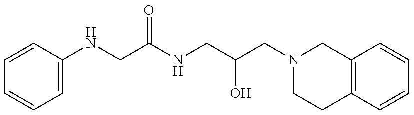 [SMILES: O=C(CNc1ccccc1)NCC(O)CN1CCc2ccccc2C1] | 339.1947 | 340.1 |
| A19 | 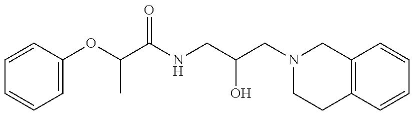 [SMILES: CC(Oc1ccccc1)C(=O)NCC(O)CN1CCc2ccccc2C1] | 354.1943 | 355.2 |
| A20 | 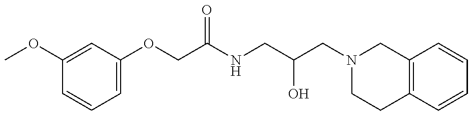 [SMILES: COc1cccc(OCC(=O)NCC(O)CN2CCc3ccccc3C2)c1] | 370.1893 | 371.1 |
| A21 | 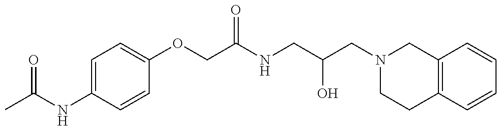 [SMILES: CC(=O)Nc1ccc(OCC(=O)NCC(O)CN2CCc3ccccc3C2)cc1] | 397.2002 | 398.2 |
| A22 | 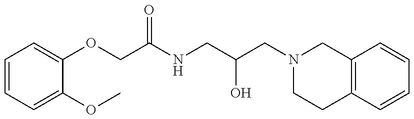 [SMILES: COc1ccccc1OCC(=O)NCC(O)CN1CCc2ccccc2C1] | 370.1893 | 371.1 |
| A23 | 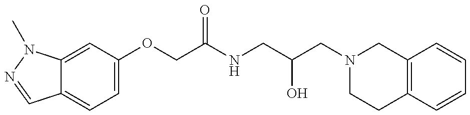 [SMILES: Cn1ncc2ccc(OCC(=O)NCC(O)CN3CCc4ccccc4C3)cc21] | 394.2005 | 395.1 |
| A24 | 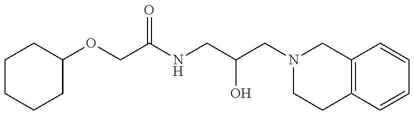 [SMILES: O=C(COC1CCCCC1)NCC(O)CN1CCc2ccccc2C1] | 346.2256 | 347.2 |
| A25 | 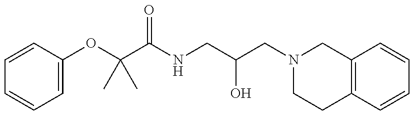 [SMILES: CC(C)(Oc1ccccc1)C(=O)NCC(O)CN1CCc2ccccc2C1] | 368.21 | 369.2 |
| A26 | 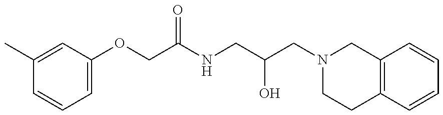 [SMILES: Cc1cccc(OCC(=O)NCC(O)CN2CCc3ccccc3C2)c1] | 354.1943 | 355.2 |
| A27 | 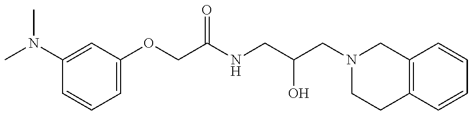 [SMILES: CN(C)c1cccc(OCC(=O)NCC(O)CN2CCc3ccccc3C2)c1] | 383.2209 | 384.2 |
| A28 | 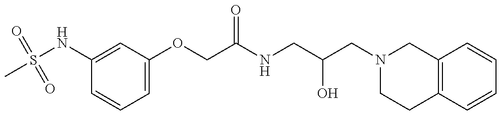 [SMILES: CS(=O)(=O)Nc1cccc(OCC(=O)NCC(O)CN2CCc3ccccc3C2)c1] | 433.1671 | 434.1 |
| A29 | 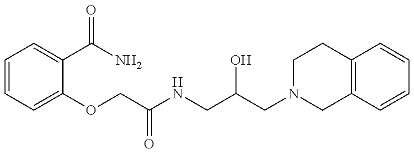 [SMILES: NC(=O)c1ccccc1OCC(=O)NCC(O)CN1CCc2ccccc2C1] | 383.1845 | 384.2 |
| A30 | 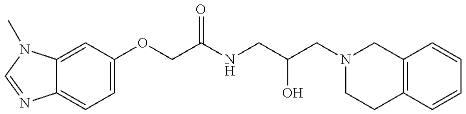 [SMILES: Cn1cnc2ccc(OCC(=O)NCC(O)CN3CCc4ccccc4C3)cc21] | 394.2005 | 395.1 |
| A31 | 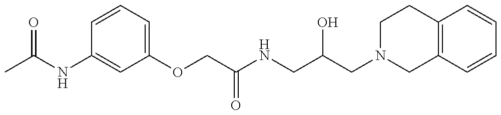 [SMILES: CC(=O)Nc1cccc(OCC(=O)NCC(O)CN2CCc3ccccc3C2)c1] | 397.2002 | 398.1 |
| A32 | 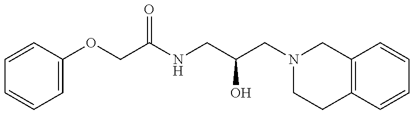 [SMILES: O=C(COc1ccccc1)NC[C@H](O)CN1CCc2ccccc2C1] | 340.1787 | 341.2 |
| A33 | 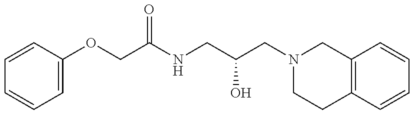 [SMILES: O=C(COc1ccccc1)NC[C@@H](O)CN1CCc2ccccc2C1] | 340.1787 | 341.2 |
| A34 | 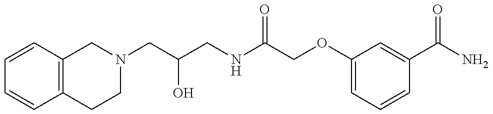 [SMILES: NC(=O)c1cccc(OCC(=O)NCC(O)CN2CCc3ccccc3C2)c1] | 383.1845 | 384.1 |
| A35 | 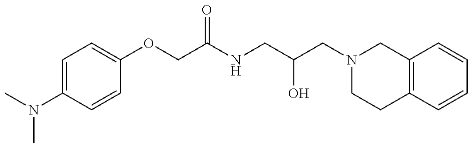 [SMILES: CN(C)c1ccc(OCC(=O)NCC(O)CN2CCc3ccccc3C2)cc1] | 383.2209 | 384.2 |
| A36 | 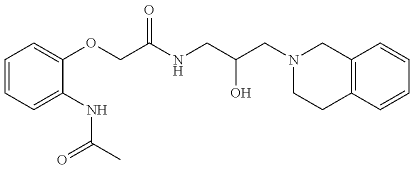 [SMILES: CC(=O)Nc1ccccc1OCC(=O)NCC(O)CN1CCc2ccccc2C1] | 397.2002 | 398.1 |
| A37 | 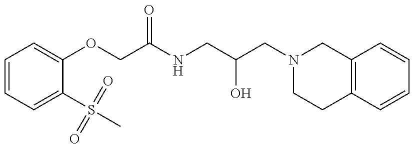 [SMILES: CS(=O)(=O)c1ccccc1OCC(=O)NCC(O)CN1CCc2ccccc2C1] | 418.1562 | 419.1 |
| A38 | 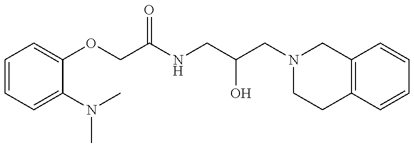 [SMILES: CN(C)c1ccccc1OCC(=O)NCC(O)CN1CCc2ccccc2C1] | 383.2209 | 384.2 |
| A39 | 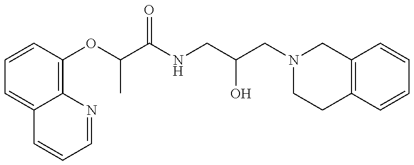 [SMILES: CC(Oc1cccc2cccnc12)C(=O)NCC(O)CN1CCc2ccccc2C1] | 405.2052 | 406.2 |
| A40 | 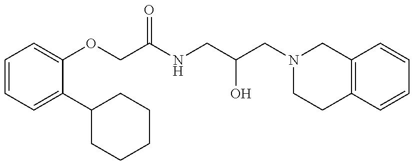 [SMILES: O=C(COc1ccccc1C1CCCCC1)NCC(O)CN1CCc2ccccc2C1] | 422.2569 | 423.2 |
| A41 | 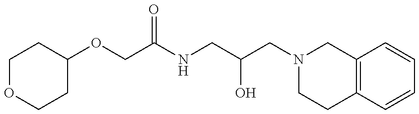 [SMILES: O=C(COC1CCOCC1)NCC(O)CN1CCc2ccccc2C1] | 348.2049 | 349.2 |
| A42 | 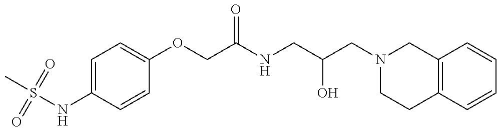 [SMILES: CS(=O)(=O)Nc1ccc(OCC(=O)NCC(O)CN2CCc3ccccc3C2)cc1] | 433.1671 | 434.1 |
| A43 | 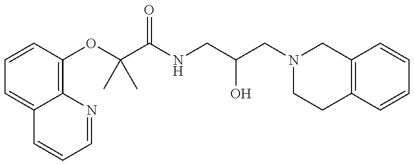 [SMILES: CC(C)(Oc1cccc2cccnc12)C(=O)NCC(O)CN1CCc2ccccc2C1] | 419.2209 | 420.2 |
| A44 | 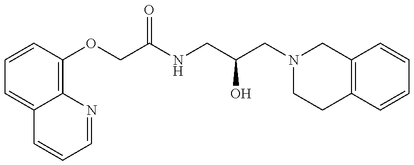 [SMILES: O=C(COc1cccc2cccnc12)NC[C@H](O)CN1CCc2ccccc2C1] | 391.1896 | 392.1 |
| A45 | 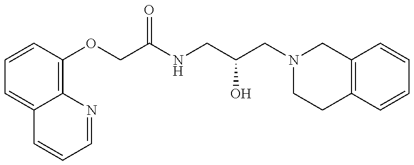 [SMILES: O=C(COc1cccc2cccnc12)NC[C@@H](O)CN1CCc2ccccc2C1] | 391.1896 | 392.1 |
| A46 | 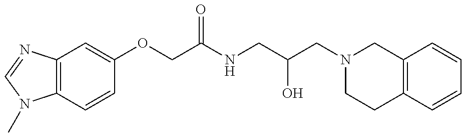 [SMILES: Cn1cnc2cc(OCC(=O)NCC(O)CN3CCc4ccccc4C3)ccc21] | 394.2005 | 395.2 |
| A47 | 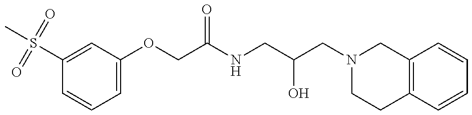 [SMILES: CS(=O)(=O)c1cccc(OCC(=O)NCC(O)CN2CCc3ccccc3C2)c1] | 418.1562 | 419.1 |
| A48 | 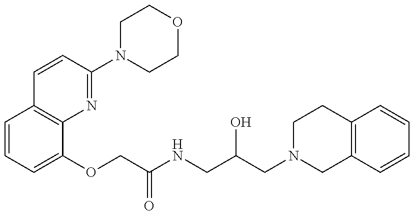 [SMILES: O=C(COc1cccc2ccc(N3CCOCC3)nc12)NCC(O)CN1CCc2ccccc2C1] | 476.2424 | 477.2 |
| A49 | 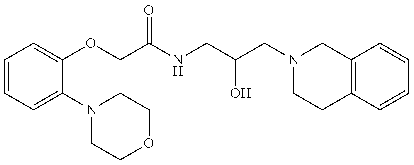 [SMILES: O=C(COc1ccccc1N1CCOCC1)NCC(O)CN1CCc2ccccc2C1] | 425.2315 | 426.2 |
| A50 | 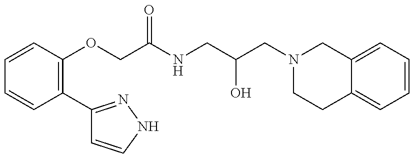 [SMILES: O=C(COc1ccccc1-c1cc[nH]n1)NCC(O)CN1CCc2ccccc2C1] | 406.2005 | 407.2 |
| A51 | 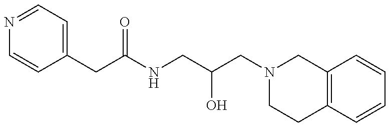 [SMILES: O=C(Cc1ccncc1)NCC(O)CN1CCc2ccccc2C1] | 325.179 | 326.1 |
| A52 | 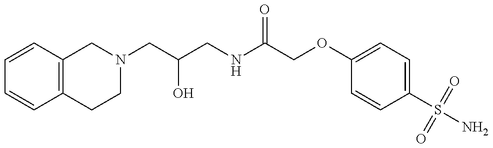 [SMILES: NS(=O)(=O)c1ccc(OCC(=O)NCC(O)CN2CCc3ccccc3C2)cc1] | 419.1515 | 420.1 |
| A53 | 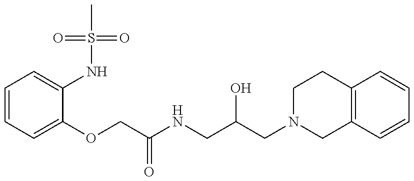 [SMILES: CS(=O)(=O)Nc1ccccc1OCC(=O)NCC(O)CN1CCc2ccccc2C1] | 433.1671 | 434.1 |
| A54 | 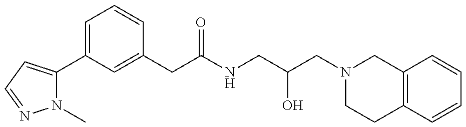 [SMILES: Cn1nccc1-c1cccc(CC(=O)NCC(O)CN2CCc3ccccc3C2)c1] | 404.2212 | 405.2 |
| A55 | 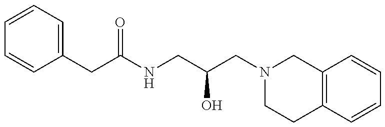 [SMILES: O=C(Cc1ccccc1)NC[C@H](O)CN1CCc2ccccc2C1] | 324.1838 | 325.1 |
| A56 | 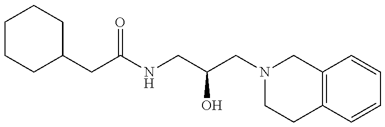 [SMILES: O=C(CC1CCCCC1)NC[C@H](O)CN1CCc2ccccc2C1] | 330.2307 | 331.2 |
| A57 | 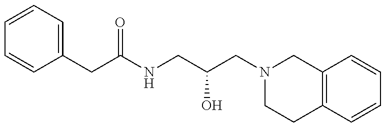 [SMILES: O=C(Cc1ccccc1)NC[C@@H](O)CN1CCc2ccccc2C1] | 324.1838 | 325.1 |
| A58 | 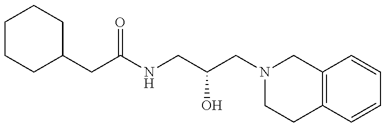 [SMILES: O=C(CC1CCCCC1)NC[C@@H](O)CN1CCc2ccccc2C1] | 330.2307 | 331.2 |
| A59 | 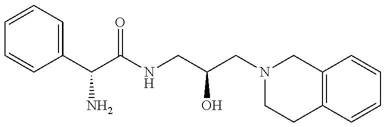 [SMILES: N[C@@H](C(=O)NC[C@H](O)CN1CCc2ccccc2C1)c1ccccc1] | 339.1947 | 340.2 |
| A60 | 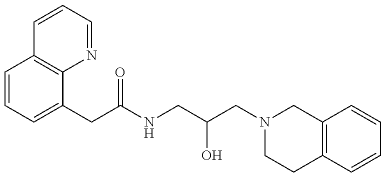 [SMILES: O=C(Cc1cccc2cccnc12)NCC(O)CN1CCc2ccccc2C1] | 375.1947 | 376.1 |
| A61 | 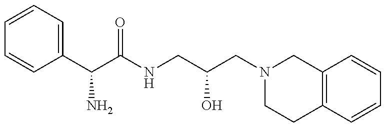 [SMILES: N[C@@H](C(=O)NC[C@@H](O)CN1CCc2ccccc2C1)c1ccccc1] | 339.1947 | 340.1 |
| A62 | 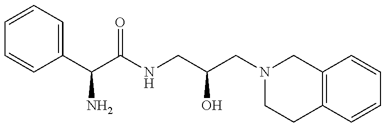 [SMILES: N[C@H](C(=O)NC[C@H](O)CN1CCc2ccccc2C1)c1ccccc1] | 339.1947 | 340.1 |
| A63 | 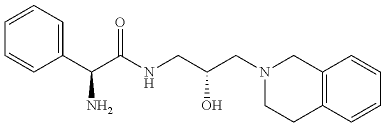 [SMILES: N[C@H](C(=O)NC[C@@H](O)CN1CCc2ccccc2C1)c1ccccc1] | 339.1947 | 340.1 |
| A64 | 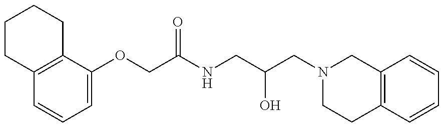 [SMILES: O=C(COc1cccc2c1CCCC2)NCC(O)CN1CCc2ccccc2C1] | 394.2256 | 395.1 |
| A65 | 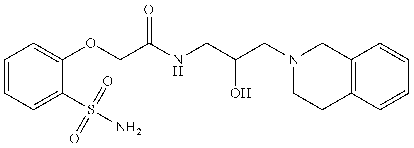 [SMILES: NS(=O)(=O)c1ccccc1OCC(=O)NCC(O)CN1CCc2ccccc2C1] | 419.1515 | 420.1 |
| A66 | 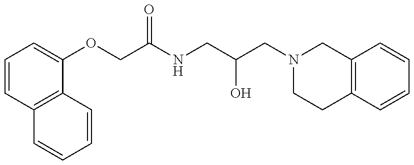 [SMILES: O=C(COc1cccc2ccccc12)NCC(O)CN1CCc2ccccc2C1] | 390.1943 | 391.2 |
| A67 | 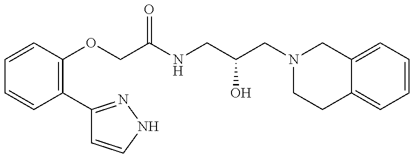 [SMILES: O=C(COc1ccccc1-c1cc[nH]n1)NC[C@@H](O)CN1CCc2ccccc2C1] | 406.2005 | 407.2 |
| A68 | 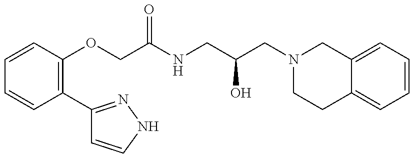 [SMILES: O=C(COc1ccccc1-c1cc[nH]n1)NC[C@H](O)CN1CCc2ccccc2C1] | 406.2005 | 407.2 |
| A69 | 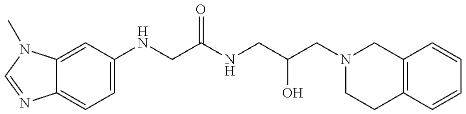 [SMILES: Cn1cnc2ccc(NCC(=O)NCC(O)CN3CCc4ccccc4C3)cc21] | 393.2165 | 394.2 |
| A70 | 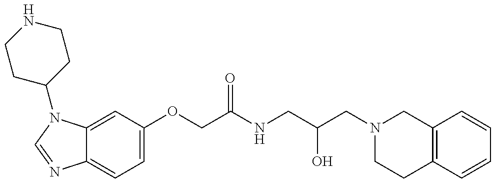 [SMILES: O=C(COc1ccc2ncn(C3CCNCC3)c2c1)NCC(O)CN1CCc2ccccc2C1] | 463.2583 | 464.2 |
| A71 | 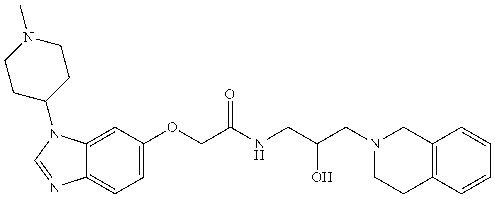 [SMILES: CN1CCC(n2cnc3ccc(OCC(=O)NCC(O)CN4CCc5ccccc5C4)cc32)CC1] | 477.274 | 478.3 |
| A72 | 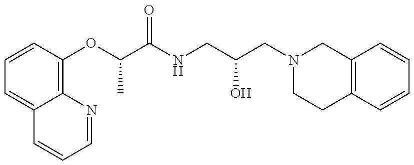 [SMILES: C[C@H](Oc1cccc2cccnc12)C(=O)NC[C@@H](O)CN1CCc2ccccc2C1] | 405.2052 | 406.2 |
| A73 | 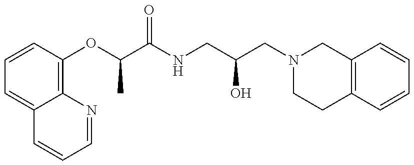 [SMILES: C[C@@H](Oc1cccc2cccnc12)C(=O)NC[C@H](O)CN1CCc2ccccc2C1] | 405.2052 | 406.1 |
| A74 | 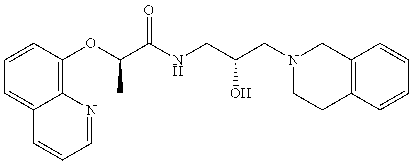 [SMILES: C[C@@H](Oc1cccc2cccnc12)C(=O)NC[C@@H](O)CN1CCc2ccccc2C1] | 405.2052 | 406.1 |
| A75 | 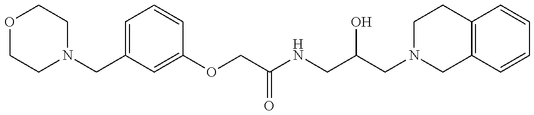 [SMILES: O=C(COc1cccc(CN2CCOCC2)c1)NCC(O)CN1CCc2ccccc2C1] | 439.2471 | 440.2 |
| A76 | 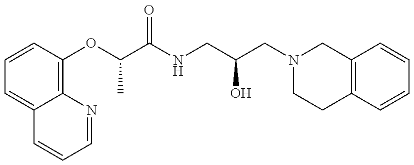 [SMILES: C[C@H](Oc1cccc2cccnc12)C(=O)NC[C@H](O)CN1CCc2ccccc2C1] | 405.2052 | 406.2 |
| A77 | 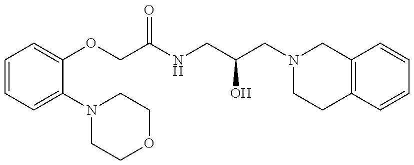 [SMILES: O=C(COc1ccccc1N1CCOCC1)NC[C@H](O)CN1CCc2ccccc2C1] | 425.2315 | 426.2 |
| A78 | 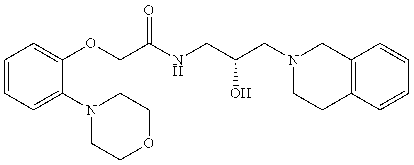 [SMILES: O=C(COc1ccccc1N1CCOCC1)NC[C@@H](O)CN1CCc2ccccc2C1] | 425.2315 | 426.2 |
| A79 | 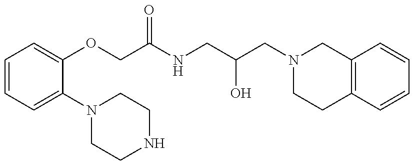 [SMILES: O=C(COc1ccccc1N1CCNCC1)NCC(O)CN1CCc2ccccc2C1] | 424.2474 | 425.2 |
| A80 | 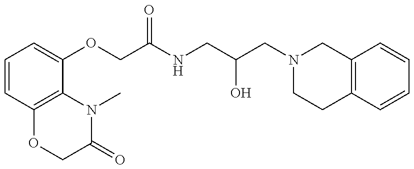 [SMILES: CN1C(=O)COc2cccc(OCC(=O)NCC(O)CN3CCc4ccccc4C3)c21] | 425.1951 | 426.2 |
| A81 | 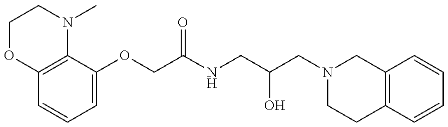 [SMILES: CN1CCOc2cccc(OCC(=O)NCC(O)CN3CCc4ccccc4C3)c21] | 411.2158 | 412.1 |
| A82 | 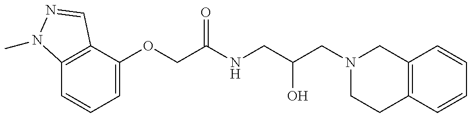 [SMILES: Cn1ncc2c(OCC(=O)NCC(O)CN3CCc4ccccc4C3)cccc21] | 394.2005 | 395.2 |
| A83 | 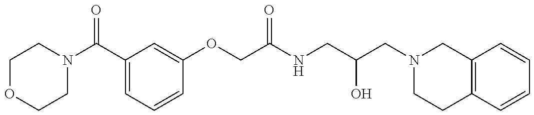 [SMILES: O=C(COc1cccc(C(=O)N2CCOCC2)c1)NCC(O)CN1CCc2ccccc2C1] | 453.2264 | 454.2 |
| A84 | 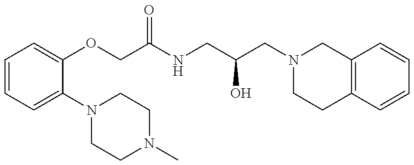 [SMILES: CN1CCN(c2ccccc2OCC(=O)NC[C@H](O)CN2CCc3ccccc3C2)CC1] | 438.2631 | 439.3 |
| A85 | 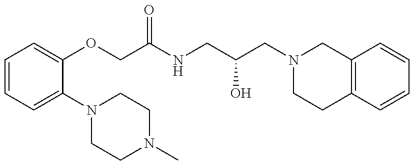 [SMILES: CN1CCN(c2ccccc2OCC(=O)NC[C@@H](O)CN2CCc3ccccc3C2)CC1] | 438.2631 | 439.3 |
| A86 | 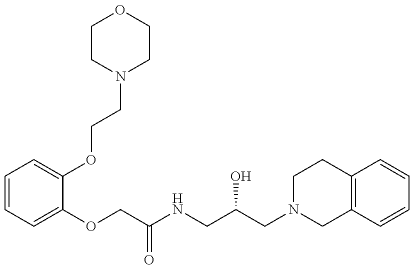 [SMILES: O=C(COc1ccccc1OCCN1CCOCC1)NC[C@H](O)CN1CCc2ccccc2C1] | 469.2577 | 470.2 |
| A87 | 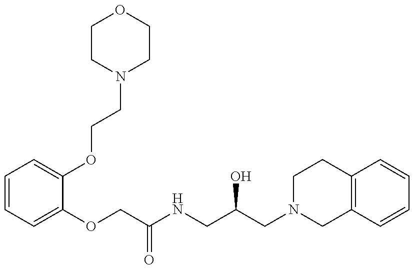 [SMILES: O=C(COc1ccccc1OCCN1CCOCC1)NC[C@@H](O)CN1CCc2ccccc2C1] | 469.2577 | 470.2 |
| A88 | 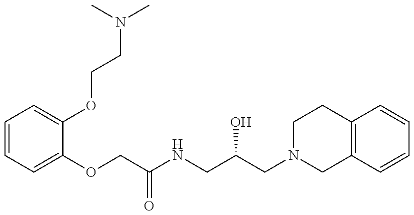 [SMILES: CN(C)CCOc1ccccc1OCC(=O)NC[C@H](O)CN1CCc2ccccc2C1] | 427.2471 | 428.1 |
| A89 | 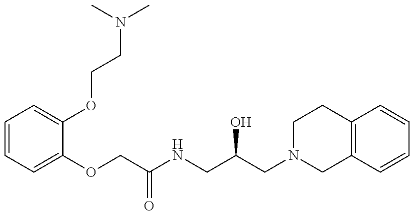 [SMILES: CN(C)CCOc1ccccc1OCC(=O)NC[C@@H](O)CN1CCc2ccccc2C1] | 427.2471 | 428.2 |
| A90 | 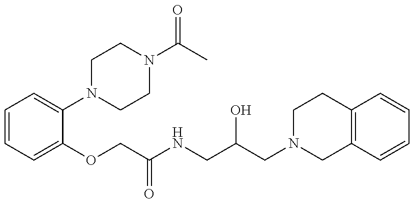 [SMILES: CC(=O)N1CCN(c2ccccc2OCC(=O)NCC(O)CN2CCc3ccccc3C2)CC1] | 466.258 | 467.2 |
| A91 | 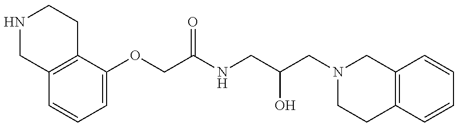 [SMILES: O=C(COc1cccc2c1CCNC2)NCC(O)CN1CCc2ccccc2C1] | 395.2209 | 396.1 |
| A92 | 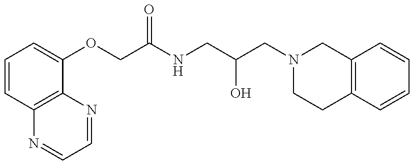 [SMILES: O=C(COc1cccc2nccnc12)NCC(O)CN1CCc2ccccc2C1] | 392.1848 | 393.2 |
| A93 | 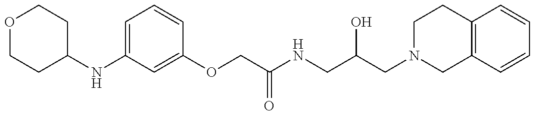 [SMILES: O=C(COc1cccc(NC2CCOCC2)c1)NCC(O)CN1CCc2ccccc2C1] | 439.2471 | 440.2 |
| A94 | 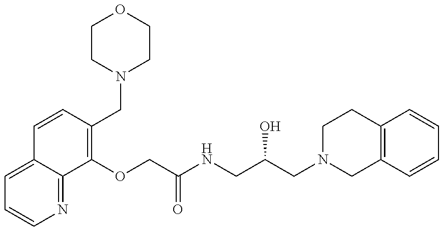 [SMILES: O=C(COc1c(CN2CCOCC2)ccc2cccnc12)NC[C@H](O)CN1CCc2ccccc2C1] | 490.258 | 491.2 |
| A95 | 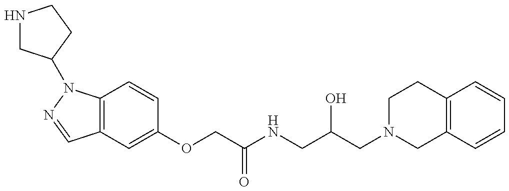 [SMILES: O=C(COc1ccc2c(cnn2C2CCNC2)c1)NCC(O)CN1CCc2ccccc2C1] | 449.2427 | 450.2 |
| A96 | 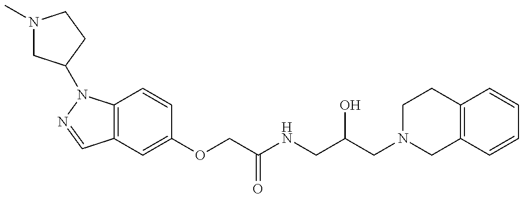 [SMILES: CN1CCC(n2ncc3cc(OCC(=O)NCC(O)CN4CCc5ccccc5C4)ccc32)C1] | 463.2583 | 464.2 |
| A97 | 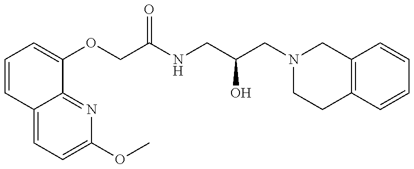 [SMILES: COc1ccc2cccc(OCC(=O)NC[C@H](O)CN3CCc4ccccc4C3)c2n1] | 421.2002 | 422.2 |
| A98 | 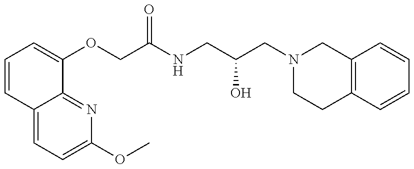 [SMILES: COc1ccc2cccc(OCC(=O)NC[C@@H](O)CN3CCc4ccccc4C3)c2n1] | 421.2002 | 422.2 |
| A99 | 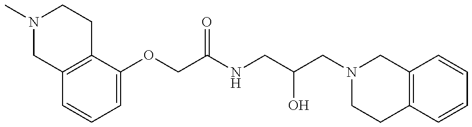 [SMILES: CN1CCc2c(cccc2OCC(=O)NCC(O)CN2CCc3ccccc3C2)C1] | 409.2365 | 410.2 |
| A100 | 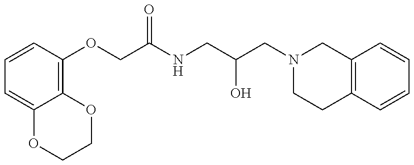 [SMILES: O=C(COc1cccc2c1OCCO2)NCC(O)CN1CCc2ccccc2C1] | 398.1842 | 399.1 |
| A101 | 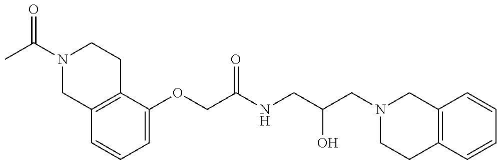 [SMILES: CC(=O)N1CCc2c(cccc2OCC(=O)NCC(O)CN2CCc3ccccc3C2)C1] | 437.2315 | 438.2 |
| A102 | 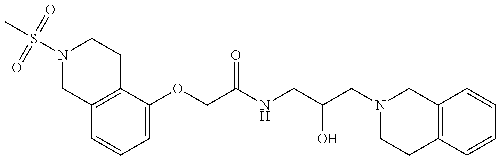 [SMILES: CS(=O)(=O)N1CCc2c(cccc2OCC(=O)NCC(O)CN2CCc3ccccc3C2)C1] | 473.1984 | 474.2 |
| A103 | 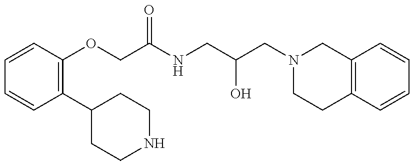 [SMILES: O=C(COc1ccccc1C1CCNCC1)NCC(O)CN1CCc2ccccc2C1] | 423.2522 | 424.3 |
| A104 | 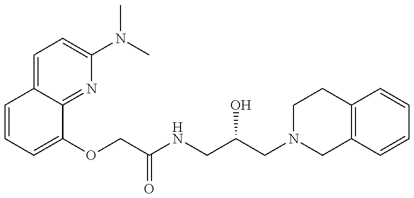 [SMILES: CN(C)c1ccc2cccc(OCC(=O)NC[C@H](O)CN3CCc4ccccc4C3)c2n1] | 434.2318 | 435.2 |
| A105 | 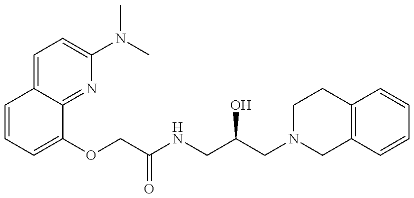 [SMILES: CN(C)c1ccc2cccc(OCC(=O)NC[C@@H](O)CN3CCc4ccccc4C3)c2n1] | 434.2318 | 435.2 |
| A106 | 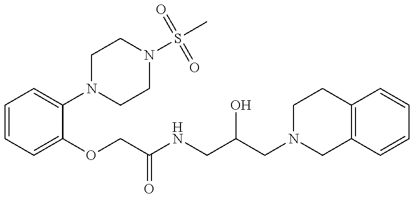 [SMILES: CS(=O)(=O)N1CCN(c2ccccc2OCC(=O)NCC(O)CN2CCc3ccccc3C2)CC1] | 502.225 | 503.2 |
| A107 | 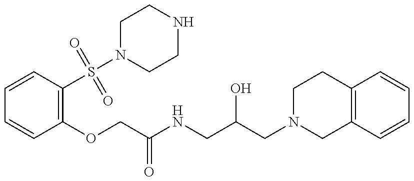 [SMILES: O=C(COc1ccccc1S(=O)(=O)N1CCNCC1)NCC(O)CN1CCc2ccccc2C1] | 488.2093 | 489.2 |
| A108 | 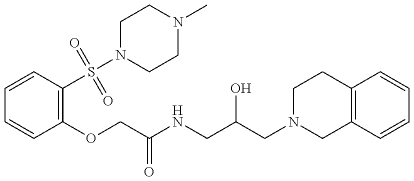 [SMILES: CN1CCN(S(=O)(=O)c2ccccc2OCC(=O)NCC(O)CN2CCc3ccccc3C2)CC1] | 502.225 | 503.2 |
| A109 | 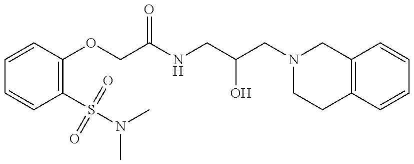 [SMILES: CN(C)S(=O)(=O)c1ccccc1OCC(=O)NCC(O)CN1CCc2ccccc2C1] | 447.1828 | 448.1 |
| A110 | 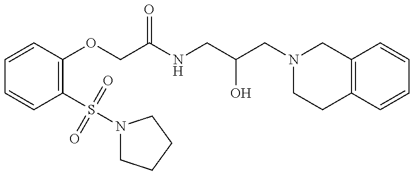 [SMILES: O=C(COc1ccccc1S(=O)(=O)N1CCCC1)NCC(O)CN1CCc2ccccc2C1] | 473.1984 | 474.1 |
| A111 | 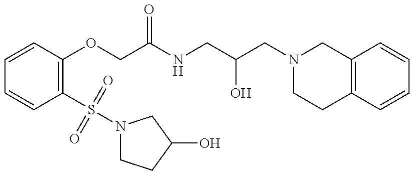 [SMILES: O=C(COc1ccccc1S(=O)(=O)N1CCC(O)C1)NCC(O)CN1CCc2ccccc2C1] | 489.1934 | 490.1 |
| A112 | 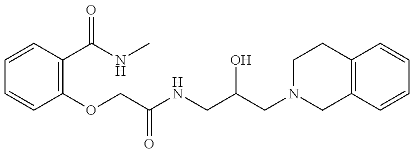 [SMILES: CNC(=O)c1ccccc1OCC(=O)NCC(O)CN1CCc2ccccc2C1] | 397.2002 | 398.2 |
| A113 | 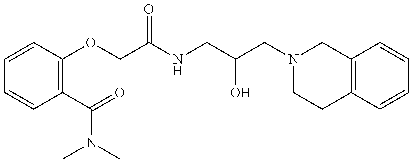 [SMILES: CN(C)C(=O)c1ccccc1OCC(=O)NCC(O)CN1CCc2ccccc2C1] | 411.2158 | 412.2 |
| A114 | 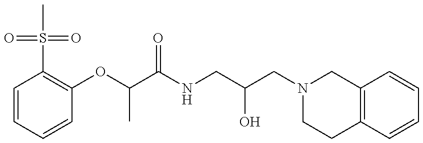 [SMILES: CC(Oc1ccccc1S(C)(=O)=O)C(=O)NCC(O)CN1CCc2ccccc2C1] | 432.1719 | 433.1 |
| A115 | 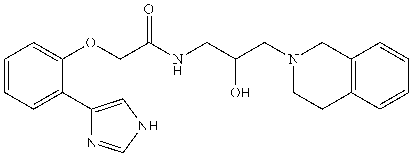 [SMILES: O=C(COc1ccccc1-c1c[nH]cn1)NCC(O)CN1CCc2ccccc2C1] | 406.2005 | 407.1 |
| A116 | 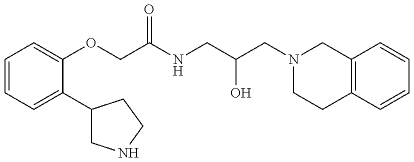 [SMILES: O=C(COc1ccccc1C1CCNC1)NCC(O)CN1CCc2ccccc2C1] | 409.2365 | 410.2 |
| A117 | 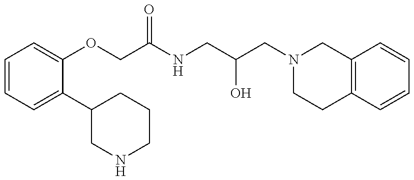 [SMILES: O=C(COc1ccccc1C1CCCNC1)NCC(O)CN1CCc2ccccc2C1] | 423.2522 | 424.2 |
| A118 | 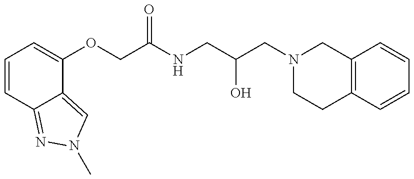 [SMILES: Cn1cc2c(OCC(=O)NCC(O)CN3CCc4ccccc4C3)cccc2n1] | 394.2005 | 395.2 |
| A119 | 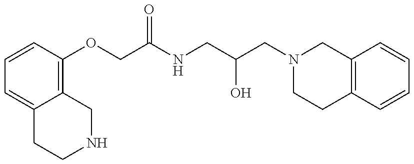 [SMILES: O=C(COc1cccc2c1CNCC2)NCC(O)CN1CCc2ccccc2C1] | 395.2209 | 396.1 |
| A120 | 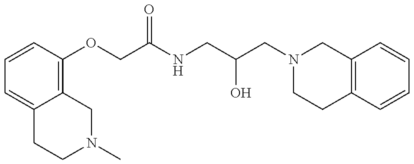 [SMILES: CN1CCc2cccc(OCC(=O)NCC(O)CN3CCc4ccccc4C3)c2C1] | 409.2365 | 410.2 |
| A121 | 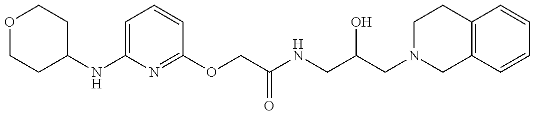 [SMILES: O=C(COc1cccc(NC2CCOCC2)n1)NCC(O)CN1CCc2ccccc2C1] | 440.2424 | 441.2 |
| A122 | 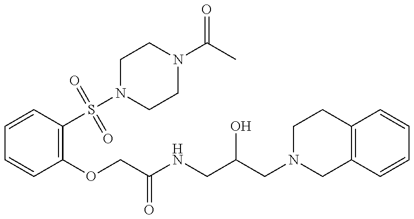 [SMILES: CC(=O)N1CCN(S(=O)(=O)c2ccccc2OCC(=O)NCC(O)CN2CCc3ccccc3C2)CC1] | 530.2199 | 531.2 |
| A123 | 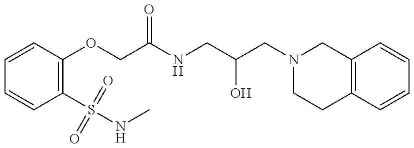 [SMILES: CNS(=O)(=O)c1ccccc1OCC(=O)NCC(O)CN1CCc2ccccc2C1] | 433.1671 | 434.1 |
| A124 | 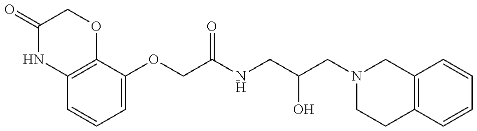 [SMILES: O=C(COc1cccc2c1OCC(=O)N2)NCC(O)CN1CCc2ccccc2C1] | 411.1794 | 412.2 |
| A125 | 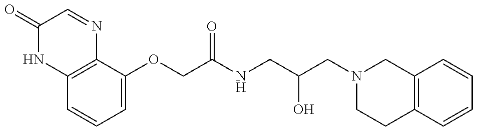 [SMILES: O=C(COc1cccc2[nH]c(=O)cnc12)NCC(O)CN1CCc2ccccc2C1] | 408.1798 | 409.2 |
| A126 | 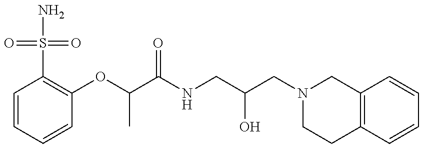 [SMILES: CC(Oc1ccccc1S(N)(=O)=O)C(=O)NCC(O)CN1CCc2ccccc2C1] | 433.1671 | 434.1 |
| A127 | 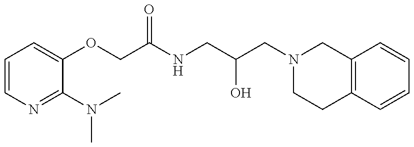 [SMILES: CN(C)c1ncccc1OCC(=O)NCC(O)CN1CCc2ccccc2C1] | 384.2161 | 385.1 |
| A128 | 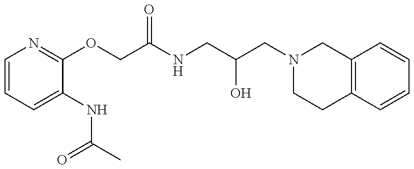 [SMILES: CC(=O)Nc1cccnc1OCC(=O)NCC(O)CN1CCc2ccccc2C1] | 398.1954 | 399.1 |
| A129 | 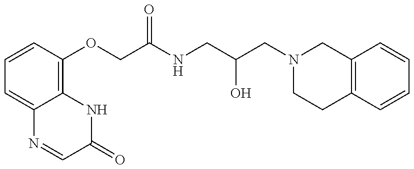 [SMILES: O=C(COc1cccc2ncc(=O)[nH]c12)NCC(O)CN1CCc2ccccc2C1] | 408.1798 | 409.1 |
| A130 | 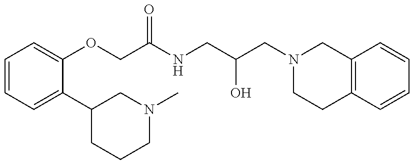 [SMILES: CN1CCCC(c2ccccc2OCC(=O)NCC(O)CN2CCc3ccccc3C2)C1] | 437.2678 | 438.3 |
| A131 | 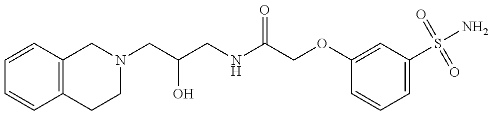 [SMILES: NS(=O)(=O)c1cccc(OCC(=O)NCC(O)CN2CCc3ccccc3C2)c1] | 419.1515 | 420.1 |
| A132 | 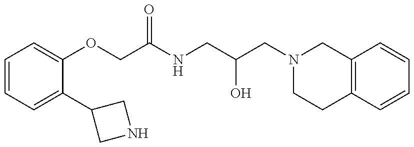 [SMILES: O=C(COc1ccccc1C1CNC1)NCC(O)CN1CCc2ccccc2C1] | 395.2209 | 396.2 |
| A133 | 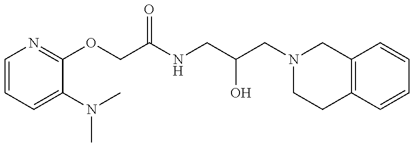 [SMILES: CN(C)c1cccnc1OCC(=O)NCC(O)CN1CCc2ccccc2C1] | 384.2161 | 385.2 |
| A134 | 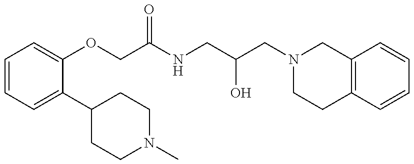 [SMILES: CN1CCC(c2ccccc2OCC(=O)NCC(O)CN2CCc3ccccc3C2)CC1] | 437.2678 | 438.3 |
| A135 | 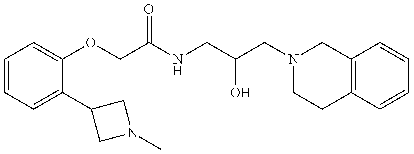 [SMILES: CN1CC(c2ccccc2OCC(=O)NCC(O)CN2CCc3ccccc3C2)C1] | 409.2365 | 410.3 |
| A136 | 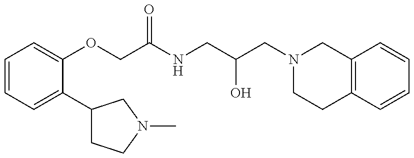 [SMILES: CN1CCC(c2ccccc2OCC(=O)NCC(O)CN2CCc3ccccc3C2)C1] | 423.2522 | 424.2 |
| A137 | 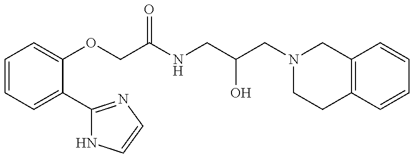 [SMILES: O=C(COc1ccccc1-c1ncc[nH]1)NCC(O)CN1CCc2ccccc2C1] | 406.2005 | 407.1 |
| A138 | 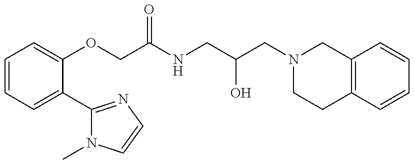 [SMILES: Cn1ccnc1-c1ccccc1OCC(=O)NCC(O)CN1CCc2ccccc2C1] | 420.2161 | 421.1 |
| A139 | 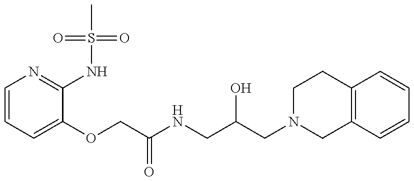 [SMILES: CS(=O)(=O)Nc1ncccc1OCC(=O)NCC(O)CN1CCc2ccccc2C1] | 434.1624 | 435.1 |
| A140 | 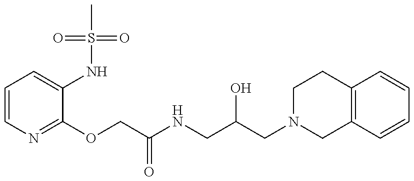 [SMILES: CS(=O)(=O)Nc1cccnc1OCC(=O)NCC(O)CN1CCc2ccccc2C1] | 434.1624 | 435.1 |
| A141 | 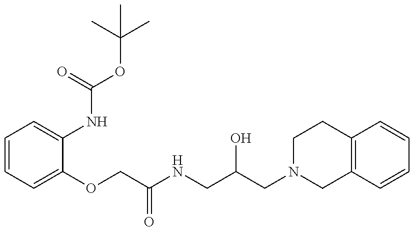 [SMILES: CC(C)(C)OC(=O)Nc1ccccc1OCC(=O)NCC(O)CN1CCc2ccccc2C1] | 455.242 | 456.2 |
| A142 | 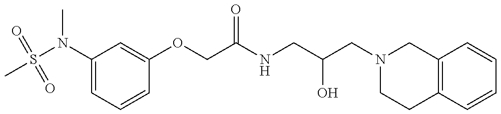 [SMILES: CN(c1cccc(OCC(=O)NCC(O)CN2CCc3ccccc3C2)c1)S(C)(=O)=O] | 447.1818 | 448.2 |
| A143 | 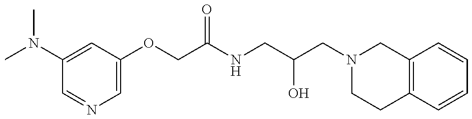 [SMILES: CN(C)c1cncc(OCC(=O)NCC(O)CN2CCc3ccccc3C2)c1] | 384.2161 | 385.2 |
| A144 | 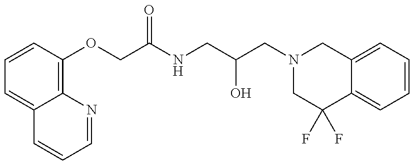 [SMILES: O=C(COc1cccc2cccnc12)NCC(O)CN1Cc2ccccc2C(F)(F)C1] | 427.1707 | 428.2 |
| A145 | 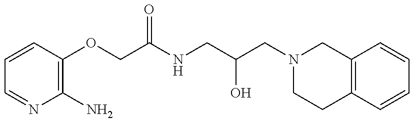 [SMILES: Nc1ncccc1OCC(=O)NCC(O)CN1CCc2ccccc2C1] | 356.1848 | 357.1 |
| A146 | 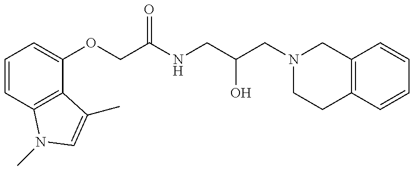 [SMILES: Cc1cn(C)c2cccc(OCC(=O)NCC(O)CN3CCc4ccccc4C3)c12] | 407.2209 | 408.2 |
| A147 | 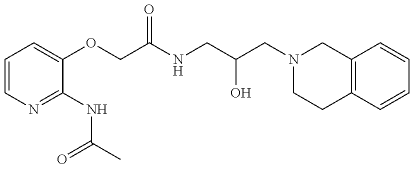 [SMILES: CC(=O)Nc1ncccc1OCC(=O)NCC(O)CN1CCc2ccccc2C1] | 398.1954 | 399.2 |
| A148 | 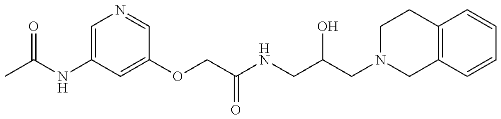 [SMILES: CC(=O)Nc1cncc(OCC(=O)NCC(O)CN2CCc3ccccc3C2)c1] | 398.1954 | 399.2 |
| A149 | 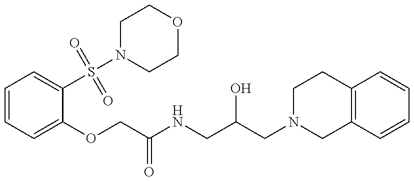 [SMILES: O=C(COc1ccccc1S(=O)(=O)N1CCOCC1)NCC(O)CN1CCc2ccccc2C1] | 489.1934 | 490.1 |
| A150 | 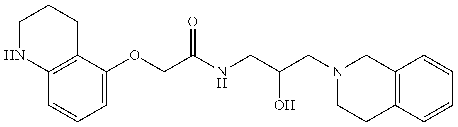 [SMILES: O=C(COc1cccc2c1CCCN2)NCC(O)CN1CCc2ccccc2C1] | 395.2209 | 396.2 |
| A151 | 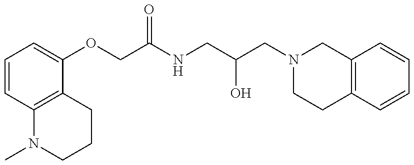 [SMILES: CN1CCCc2c(OCC(=O)NCC(O)CN3CCc4ccccc4C3)cccc21] | 409.2365 | 410.2 |
| A152 | 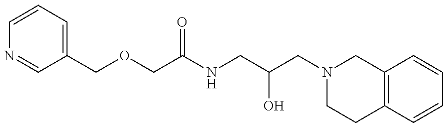 [SMILES: O=C(COCc1cccnc1)NCC(O)CN1CCc2ccccc2C1] | 355.1896 | 356.2 |

In certain embodiments, a provided compound is a compound listed in Table B-1, or a pharmaceutically acceptable salt thereof

TABLE B-1
Exemplary Compounds

| Cmpd | | | LCMS m/z |
|---|---|---|---|
| No | Structure | Exact Mass | (M + H) |
| B1. | 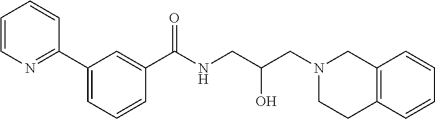 [SMILES: O=C(NCC(O)CN1CCc2ccccc2C1)c1cccc(-c2ccccn2)c1] | 387.1947 | 388.2 |
| B2. | 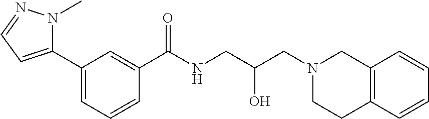 [SMILES: Cn1nccc1-c1cccc(C(=O)NCC(O)CN2CCc3ccccc3C2)c1] | 390.2056 | 391.2 |
| B3. | 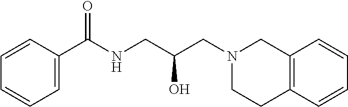 [SMILES: O=C(NC[C@H](O)CN1CCc2ccccc2C1)c1ccccc1] | 310.1681 | 311.1 |
| B4. | 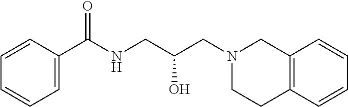 [SMILES: O=C(NC[C@@H](O)CN1CCc2ccccc2C1)c1ccccc1] | 310.1681 | 311.1 |
| B5. | 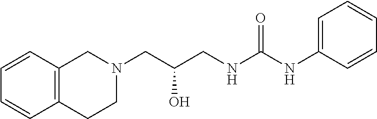 [SMILES: O=C(NC[C@H](O)CN1CCc2ccccc2C1)Nc1ccccc1] | 325.179 | 326.2 |
| B6. | 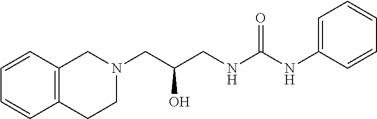 [SMILES: O=C(NC[C@@H](O)CN1CCc2ccccc2C1)Nc1ccccc1] | 325.179 | 326.2 |
| B7. | 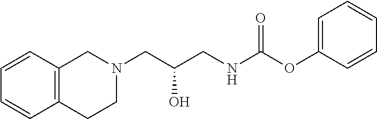 [SMILES: O=C(NC[C@H](O)CN1CCc2ccccc2C1)Oc1ccccc1] | 326.163 | 327.2 |
| B8. | 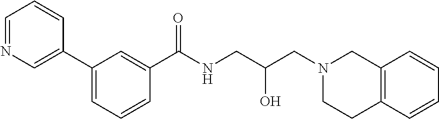 [SMILES: O=C(NCC(O)CN1CCc2ccccc2C1)c1cccc(-c2cccnc2)c1] | 387.1947 | 388.2 |
| B9. | 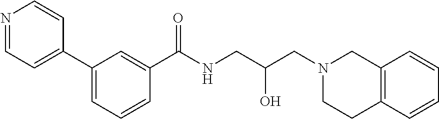 [SMILES: O=C(NCC(O)CN1CCc2ccccc2C1)c1cccc(-c2ccncc2)c1] | 387.1947 | 388.2 |
| B10. | 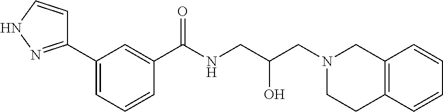 [SMILES: O=C(NCC(O)CN1CCc2ccccc2C1)c1cccc(-c2cc[nH]n2)c1] | 376.1899 | 377.2 |
| B11. | 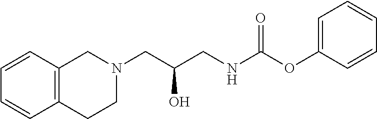 [SMILES: O=C(NC[C@@H](O)CN1CCc2ccccc2C1)Oc1ccccc1] | 326.163 | 327.2 |
| B12. | 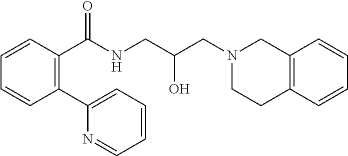 [SMILES: O=C(NCC(O)CN1CCc2ccccc2C1)c1ccccc1-c1ccccn1] | 387.1947 | 388.2 |
| B13. | 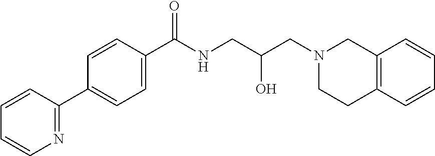 [SMILES: O=C(NCC(O)CN1CCc2ccccc2C1)c1ccc(-c2ccccn2)cc1] | 387.1947 | 388.2 |
| B14. | 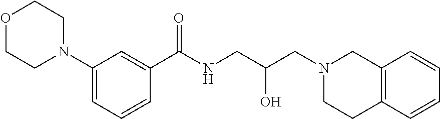 [SMILES: O=C(NCC(O)CN1CCc2ccccc2C1)c1cccc(N2CCOCC2)c1] | 395.2209 | 396.2 |
| B15. | 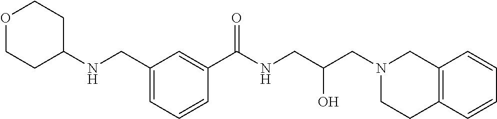 [SMILES: O=C(NCC(O)CN1CCc2ccccc2C1)c1cccc(CNC2CCOCC2)c1] | 423.2522 | 424.2 |
| B16. | 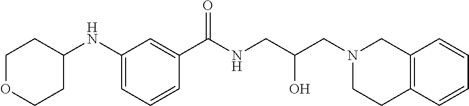 [SMILES: O=C(NCC(O)CN1CCc2ccccc2C1)c1cccc(NC2CCOCC2)c1] | 409.2365 | 410.2 |
| B17. | 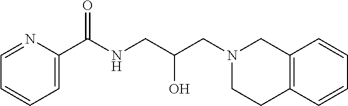 [SMILES: O=C(NCC(O)CN1CCc2ccccc2C1)c1ccccn1] | 311.1634 | 312.1 |
| B18. | 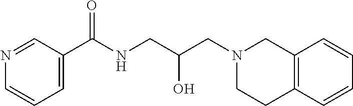 [SMILES: O=C(NCC(O)CN1CCc2ccccc2C1)c1cccnc1] | 311.1634 | 312.2 |
| B19. | 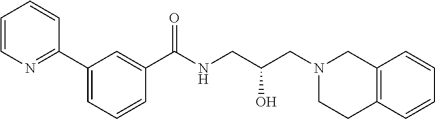 [SMILES: O=C(NC[C@@H](O)CN1CCc2ccccc2C1)c1cccc(-c2ccccn2)c1] | 387.1947 | 388.2 |
| B20. | 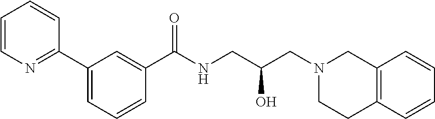 [SMILES: O=C(NC[C@H](O)CN1CCc2ccccc2C1)c1cccc(-c2ccccn2)c1] | 387.1947 | 388.2 |
| B21. | 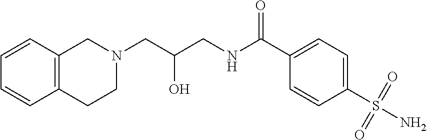 [SMILES: NS(=O)(=O)c1ccc(C(=O)NCC(O)CN2CCc3ccccc3C2)cc1] | 389.1409 | 390.1 |
| B22. | 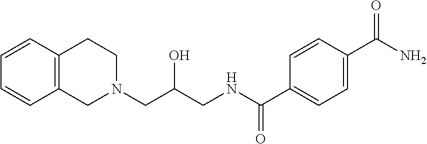 [SMILES: NC(=O)c1ccc(C(=O)NCC(O)CN2CCc3ccccc3C2)cc1] | 353.1739 | 354.1 |
| B23. | 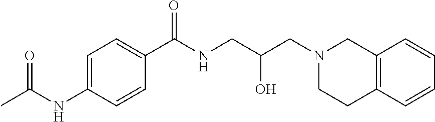 [SMILES: CC(=O)Nc1ccc(C(=O)NCC(O)CN2CCc3ccccc3C2)cc1] | 367.1896 | 368.1 |
| B24. | 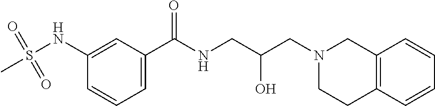 [SMILES: CS(=O)(=O)Nc1cccc(C(=O)NCC(O)CN2CCc3ccccc3C2)c1] | 403.1566 | 404.1 |
| B25. | 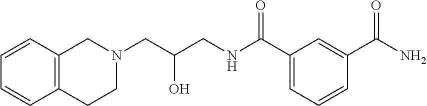 [SMILES: NC(=O)c1cccc(C(=O)NCC(O)CN2CCc3ccccc3C2)c1] | 353.1739 | 354.2 |
| B26. | 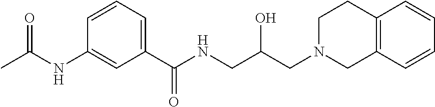 [SMILES: CC(=O)Nc1cccc(C(=O)NCC(O)CN2CCc3ccccc3C2)c1] | 367.1896 | 368.2 |
| B27. | 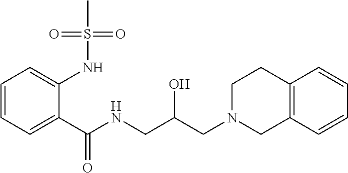 [SMILES: CS(=O)(=O)Nc1ccccc1C(=O)NCC(O)CN1CCc2ccccc2C1] | 403.1566 | 404.2 |
| B28. | 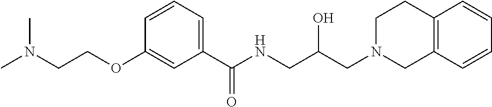 [SMILES: CN(C)CCOc1cccc(C(=O)NCC(O)CN2CCc3ccccc3C2)c1] | 397.2365 | 398.1 |
| B29. | 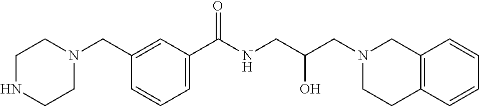 [SMILES: O=C(NCC(O)CN1CCc2ccccc2C1)c1cccc(CN2CCNCC2)c1] | 408.2525 | 409.2 |
| B30. | 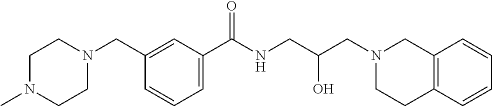 [SMILES: CN1CCN(Cc2cccc(C(=O)NCC(O)CN3CCc4ccccc4C3)c2)CC1] | 422.2682 | 423.2 |
| B31. | 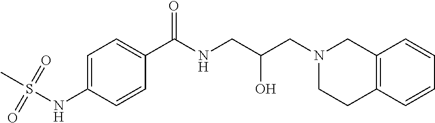 [SMILES: CS(=O)(=O)Nc1ccc(C(=O)NCC(O)CN2CCc3ccccc3C2)cc1] | 403.1566 | 404.2 |
| B32. | 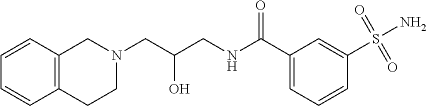 [SMILES: NS(=O)(=O)c1cccc(C(=O)NCC(O)CN2CCc3ccccc3C2)c1] | 389.1409 | 390.1 |
| B33. | 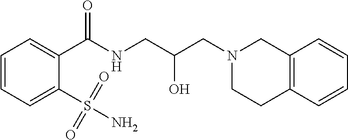 [SMILES: NS(=O)(=O)c1ccccc1C(=O)NCC(O)CN1CCc2ccccc2C1] | 389.1409 | 390 |
| B34. | 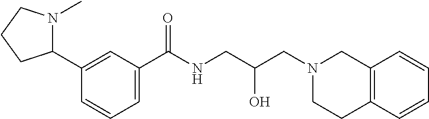 [SMILES: CN1CCCC1c1cccc(C(=O)NCC(O)CN2CCc3ccccc3C2)c1] | 393.2416 | 394.1 |
| B35. | 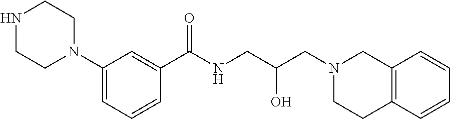 [SMILES: O=C(NCC(O)CN1CCc2ccccc2C1)c1cccc(N2CCNCC2)c1] | 394.2369 | 395.2 |
| B36. | 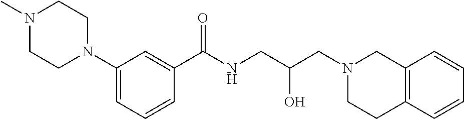 [SMILES: CN1CCN(c2cccc(C(=O)NCC(O)CN3CCc4ccccc4C3)c2)CC1] | 408.2525 | 409.2 |
| B37. | 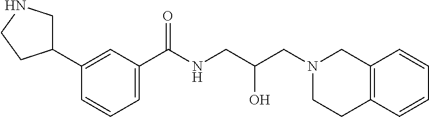 [SMILES: O=C(NCC(O)CN1CCc2ccccc2C1)c1cccc(C2CCNC2)c1] | 379.226 | 380.2 |
| B38. | 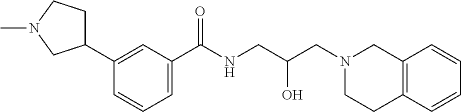 [SMILES: CN1CCC(c2cccc(C(=O)NCC(O)CN3CCc4ccccc4C3)c2)C1] | 393.2416 | 394.2 |
| B39. | 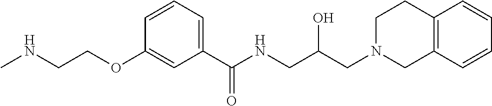 [SMILES: CNCCOc1cccc(C(=O)NCC(O)CN2CCc3ccccc3C2)c1] | 383.2209 | 384.2 |
| B40. | 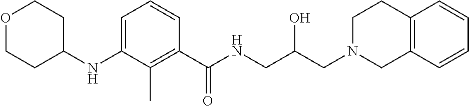 [SMILES: Cc1c(NC2CCOCC2)cccc1C(=O)NCC(O)CN1CCc2ccccc2C1] | 423.2522 | 424.2 |
| B41. | 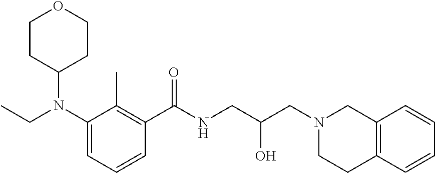 [SMILES: CCN(c1cccc(C(=O)NCC(O)CN2CCc3ccccc3C2)c1C)C1CCOCC1] | 451.2835 | 452.3 |
| B42. | 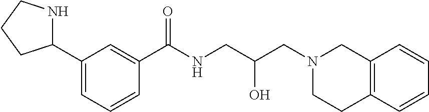 [SMILES: O=C(NCC(O)CN1CCc2ccccc2C1)c1cccc(C2CCCN2)c1] | 379.226 | 380.2 |
| B43. | 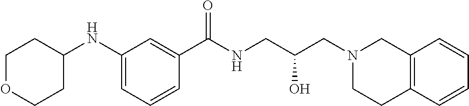 [SMILES: O=C(NC[C@@H](O)CN1CCc2ccccc2C1)c1cccc(NC2CCOCC2)c1] | 409.2365 | 410.2 |
| B44. | 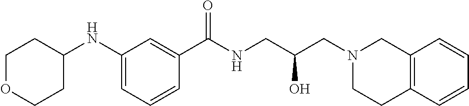 [SMILES: O=C(NC[C@H](O)CN1CCc2ccccc2C1)c1cccc(NC2CCOCC2)c1] | 409.2365 | 410.2 |
| B45. | 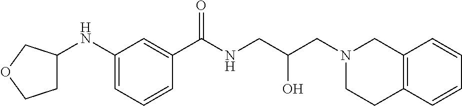 [SMILES: O=C(NCC(O)CN1CCc2ccccc2C1)c1cccc(NC2CCOC2)c1] | 395.2209 | 396.2 |
| B46. | 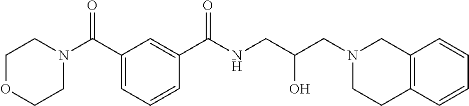 [SMILES: O=C(NCC(O)CN1CCc2ccccc2C1)c1cccc(C(=O)N2CCOCC2)c1] | 423.2158 | 424.2 |
| B47. | 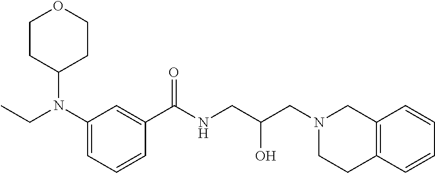 [SMILES: CCN(c1cccc(C(=O)NCC(O)CN2CCc3ccccc3C2)c1)C1CCOCC1] | 437.2678 | 438.3 |
| B48. | 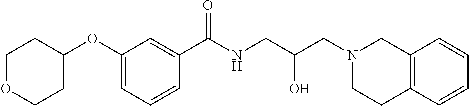 [SMILES: O=C(NCC(O)CN1CCc2ccccc2C1)c1cccc(OC2CCOCC2)c1] | 410.2206 | 411.2 |
| B49. | 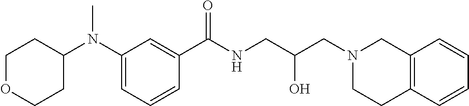 [SMILES: CN(c1cccc(C(=O)NCC(O)CN2CCc3ccccc3C2)c1)C1CCOCC1] | 423.2522 | 424.1 |
| B50. | 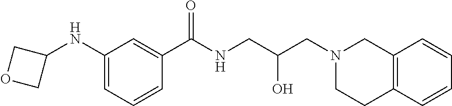 [SMILES: O=C(NCC(O)CN1CCc2ccccc2C1)c1cccc(NC2COC2)c1] | 381.2052 | 382.2 |
| B51. | 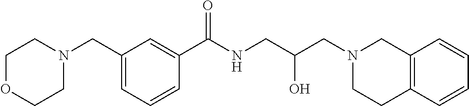 [SMILES: O=C(NCC(O)CN1CCc2ccccc2C1)c1cccc(CN2CCOCC2)c1] | 409.2365 | 410.1 |
| B52. | 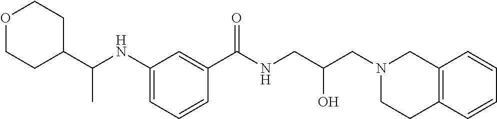 [SMILES: CC(Nc1cccc(C(=O)NCC(O)CN2CCc3ccccc3C2)c1)C1CCOCC1] | 437.2678 | 438.3 |
| B53. | 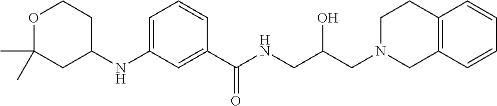 [SMILES: CC1(C)CC(Nc2cccc(C(=O)NCC(O)CN3CCc4ccccc4C3)c2)CCO1] | 437.2678 | 438.3 |
| B54. | 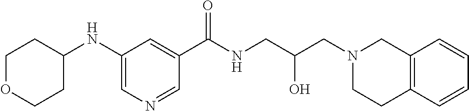 [SMILES: O=C(NCC(O)CN1CCc2ccccc2C1)c1cncc(NC2CCOCC2)c1] | 410.2318 | 411.1 |
| B55. | 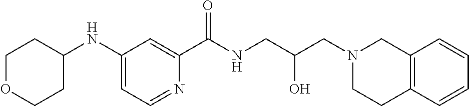 [SMILES: O=C(NCC(O)CN1CCc2ccccc2C1)c1cc(NC2CCOCC2)ccn1] | 410.2318 | 411.1 |
| B56. | 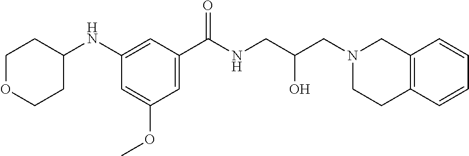 [SMILES: COc1cc(NC2CCOCC2)cc(C(=O)NCC(O)CN2CCc3ccccc3C2)c1] | 439.2471 | 440.1 |
| B57. | 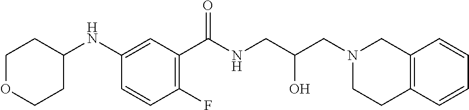 [SMILES: O=C(NCC(O)CN1CCc2ccccc2C1)c1cc(NC2CCOCC2)ccc1F] | 427.2271 | 428.2 |
| B58. | 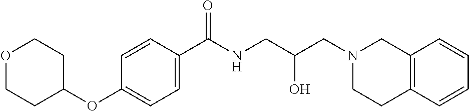 [SMILES: O=C(NCC(O)CN1CCc2ccccc2C1)c1ccc(OC2CCOCC2)cc1] | 410.2206 | 411.2 |

In certain embodiments, a provided compound is a compound listed in Table C-1, or a pharmaceutically acceptable salt thereof

TABLE C-1
Exemplary Compounds

| Cmpd | | | LCMS m/z |
|---|---|---|---|
| No | Structure | Exact Mass | (M + H) |
| C1 | 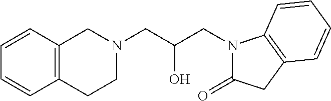 [SMILES: O=C1Cc2ccccc2N1CC(O)CN1CCc2ccccc2C1] | 322.1681 | 323.1 |
| C2 | 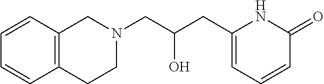 [SMILES: O=c1cccc(CC(O)CN2CCc3ccccc3C2)[nH]1] | 284.1525 | 285.1 |
| C3 | 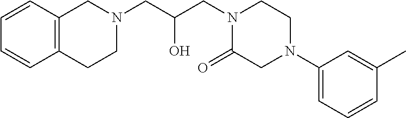 [SMILES: Cc1cccc(N2CCN(CC(O)CN3CCc4ccccc4C3)C(=O)C2)c1] | 379.226 | 380.2 |
| C4 | 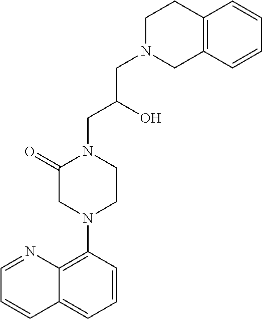 [SMILES: O=C1CN(c2cccc3cccnc23)CCN1CC(O)CN1CCc2ccccc2C1] | 416.2212 | 417.2 |
| C5 | 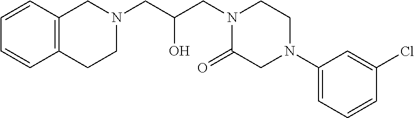 [SMILES: O=C1CN(c2cccc(Cl)c2)CCN1CC(O)CN1CCc2ccccc2C1] | 399.1714 | 400.1 |
| C6 | 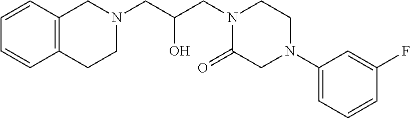 [SMILES: O=C1CN(c2cccc(F)c2)CCN1CC(O)CN1CCc2ccccc2C1] | 383.2009 | 384.2 |
| C7 | 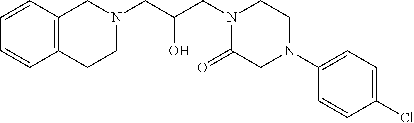 [SMILES: O=C1CN(c2ccc(Cl)cc2)CCN1CC(O)CN1CCc2ccccc2C1] | 399.1714 | 400.1 |
| C8 | 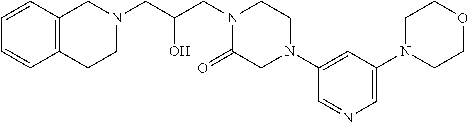 [SMILES: O=C1CN(c2cncc(N3CCOCC3)c2)CCN1CC(O)CN1CCc2ccccc2C1] | 451.2583 | 452.3 |
| C9 | 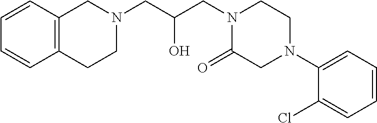 [SMILES: O=C1CN(c2ccccc2Cl)CCN1CC(O)CN1CCc2ccccc2C1] | 399.1714 | 400.1 |
| C10 | 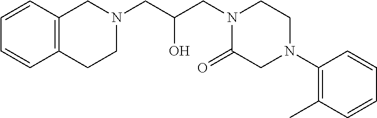 [SMILES: Cc1ccccc1N1CCN(CC(O)CN2CCc3ccccc3C2)C(=O)C1] | 379.226 | 380.2 |
| C11 | 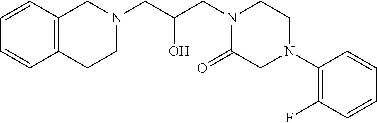 [SMILES: O=C1CN(c2ccccc2F)CCN1CC(O)CN1CCc2ccccc2C1] | 383.2009 | 384.2 |
| C12 | 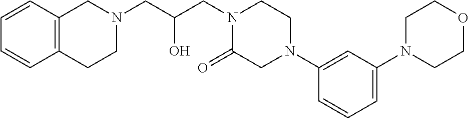 [SMILES: O=C1CN(c2cccc(N3CCOCC3)c2)CCN1CC(O)CN1CCc2ccccc2C1] | 450.2631 | 451.2 |
| C13 | 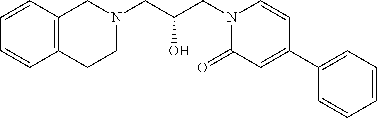 [SMILES: O=c1cc(-c2ccccc2)ccn1C[C@H](O)CN1CCc2ccccc2C1] | 360.1838 | 361.2 |
| C14 | 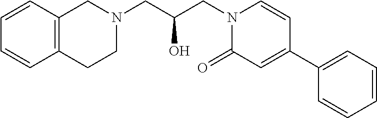 [SMILES: O=c1cc(-c2ccccc2)ccn1C[C@@H](O)CN1CCc2ccccc2C1] | 360.1838 | 361.2 |
| C15 | 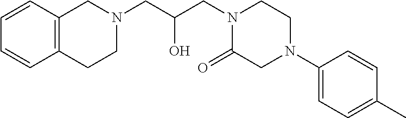 [SMILES: Cc1ccc(N2CCN(CC(O)CN3CCc4ccccc4C3)C(=O)C2)cc1] | 379.226 | 380.2 |
| C16 | 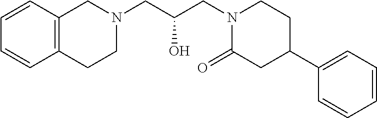 [SMILES: O=C1CC(c2ccccc2)CCN1C[C@H](O)CN1CCc2ccccc2C1] | 364.2151 | 365.2 |
| C17 | 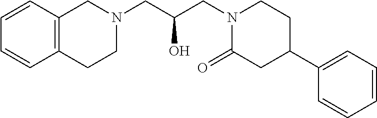 [SMILES: O=C1CC(c2ccccc2)CCN1C[C@@H](O)CN1CCc2ccccc2C1] | 364.2151 | 365.2 |
| C18 | 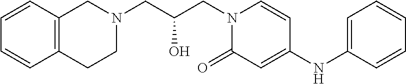 [SMILES: O=c1cc(Nc2ccccc2)ccn1C[C@H](O)CN1CCc2ccccc2C1] | 375.1947 | 376.2 |
| C19 | 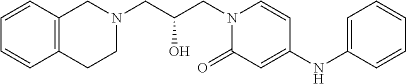 [SMILES: O=c1cc(Nc2ccccc2)ccn1C[C@H](O)CN1CCc2ccccc2C1] | 375.1947 | 376.2 |
| C20 | 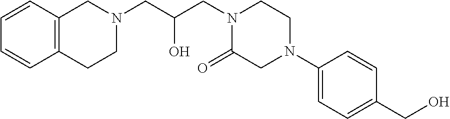 [SMILES: O=C1CN(c2ccc(CO)cc2)CCN1CC(O)CN1CCc2ccccc2C1] | 395.2209 | 396.2 |
| C21 | 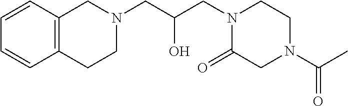 [SMILES: CC(=O)N1CCN(CC(O)CN2CCc3ccccc3C2)C(=O)C1] | 331.1896 | 332.1 |
| C22 | 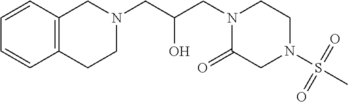 [SMILES: CS(=O)(=O)N1CCN(CC(O)CN2CCc3ccccc3C2)C(=O)C1] | 367.1566 | 368.1 |
| C23 | 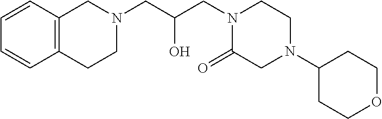 [SMILES: O=C1CN(C2CCOCC2)CCN1CC(O)CN1CCc2ccccc2C1] | 373.2365 | 374.2 |
| C24 | 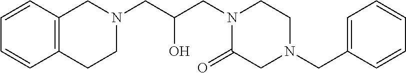 [SMILES: O=C1CN(Cc2ccccc2)CCN1CC(O)CN1CCc2ccccc2C1] | 379.226 | 380.1 |
| C25 | 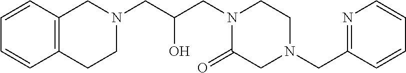 [SMILES: O=C1CN(Cc2ccccn2)CCN1CC(O)CN1CCc2ccccc2C1] | 380.2212 | 381.2 |
| C26 | 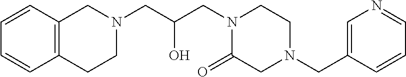 [SMILES: O=C1CN(Cc2cccnc2)CCN1CC(O)CN1CCc2ccccc2C1] | 380.2212 | 381.1 |
| C27 | 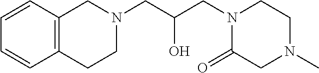 [SMILES: CN1CCN(CC(O)CN2CCc3ccccc3C2)C(=O)C1] | 303.1947 | 304.1 |
| C28 | 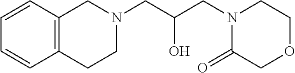 [SMILES: O=C1COCCN1CC(O)CN1CCc2ccccc2C1] | 290.163 | 291.1 |
| C29 | 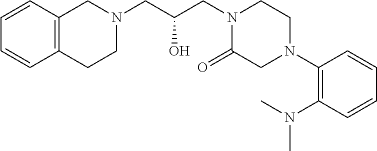 [SMILES: CN(C)c1ccccc1N1CCN(C[C@H](O)CN2CCc3ccccc3C2)C(=O)C1] | 408.2525 | 409.3 |
| C30 | 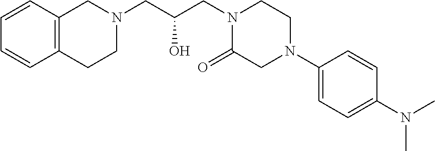 [SMILES: CN(C)c1ccc(N2CCN(C[C@H](O)CN3CCc4ccccc4C3)C(=O)C2)cc1] | 408.2525 | 407.2 |
| C31 | 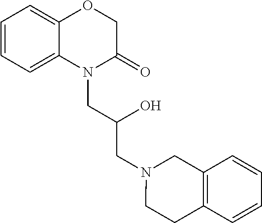 [SMILES: O=C1COc2ccccc2N1CC(O)CN1CCc2ccccc2C1] | 338.163 | 339.2 |
| C32 | 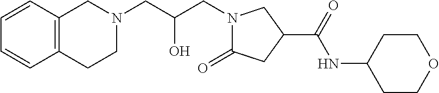 [SMILES: O=C(NC1CCOCC1)C1CC(=O)N(CC(O)CN2CCc3ccccc3C2)C1] | 401.2315 | 402.2 |

In certain embodiments, a provided compound is a compound listed in Table D-1, or a pharmaceutically acceptable salt thereof

TABLE D-1
Exemplary Compounds

| Cmpd | | | LC-MS m/z |
|---|---|---|---|
| No | Structure | Exact mass | (M + H) |
| D1. | 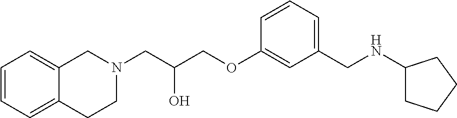 [SMILES: OC(COc1cccc(CNC2CCCC2)c1)CN1CCc2ccccc2C1] | 380.2464 | 381.2 |
| D2. | 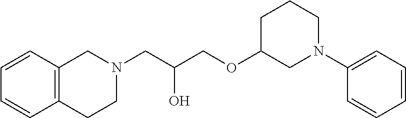 [SMILES: OC(COC1CCCN(c2ccccc2)C1)CN1CCc2ccccc2C1] | 366.2307 | 367.2 |
| D3. | 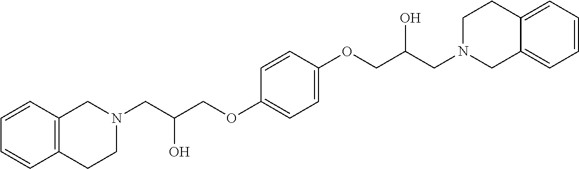 [SMILES: OC(COc1ccc(OCC(O)CN2CCc3ccccc3C2)cc1)CN1CCc2ccccc2C1] | 488.2675 | 489.2 |
| D4. | 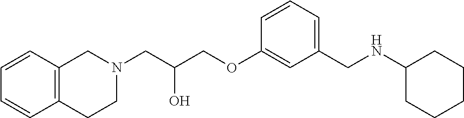 [SMILES: OC(COc1cccc(CNC2CCCCC2)c1)CN1CCc2ccccc2C1] | 394.262 | 395.0 |
| D5. | 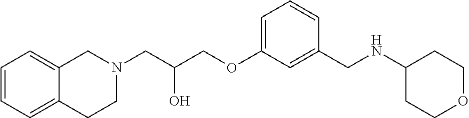 [SMILES: OC(COc1cccc(CNC2CCOCC2)c1)CN1CCc2ccccc2C1] | 396.2413 | 397.2 |
| D6. | 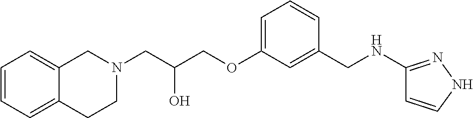 [SMILES: OC(COc1cccc(CNc2cc[nH]n2)c1)CN1CCc2ccccc2C1] | 378.2056 | 379.1 |
| D7. | 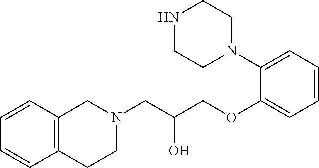 [SMILES: OC(COc1ccccc1N1CCNCC1)CN1CCc2ccccc2C1] | 367.226 | 368.1 |
| D8. | 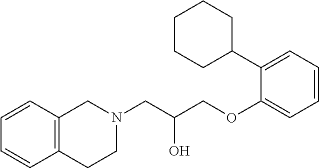 [SMILES: OC(COc1ccccc1C1CCCCC1)CN1CCc2ccccc2C1] | 365.2355 | 366.1 |
| D9. | 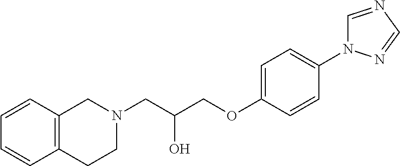 [SMILES: OC(COc1ccc(-n2cncn2)cc1)CN1CCc2ccccc2C1] | 350.1743 | 351.1 |
| D10. | 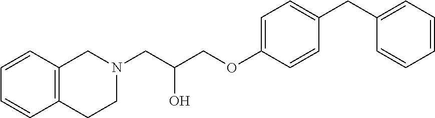 [SMILES: OC(COc1ccc(Cc2ccccc2)cc1)CN1CCc2ccccc2C1] | 373.2042 | 374.1 |
| D11. | 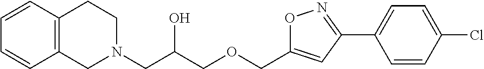 [SMILES: OC(COCc1cc(-c2ccc(Cl)cc2)no1)CN1CCc2ccccc2C1] | 398.1397 | 399.1 |
| D12. | 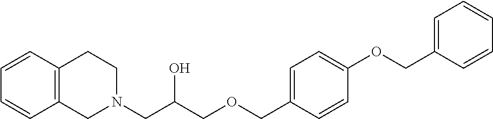 [SMILES: OC(COCc1ccc(OCc2ccccc2)cc1)CN1CCc2ccccc2C1] | 403.2147 | 403.9 |
| D13. | 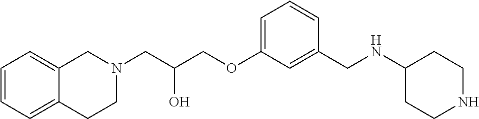 [SMILES: OC(COc1cccc(CNC2CCNCC2)c1)CN1CCc2ccccc2C1] | 395.2573 | 395.9 |
| D14. | 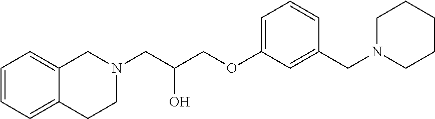 [SMILES: OC(COc1cccc(CN2CCCCC2)c1)CN1CCc2ccccc2C1] | 380.2464 | 381.0 |
| D15. | 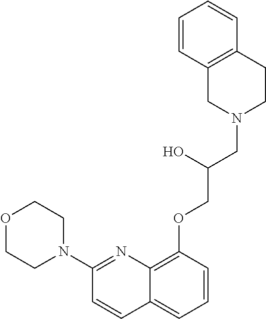 [SMILES: OC(COc1cccc2ccc(N3CCOCC3)nc12)CN1CCc2ccccc2C1] | 419.2209 | 420.2 |
| D16. | 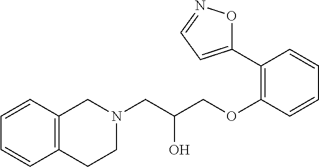 [SMILES: OC(COc1ccccc1-c1ccno1)CN1CCc2ccccc2C1] | 350.163 | 351.0 |
| D17. | 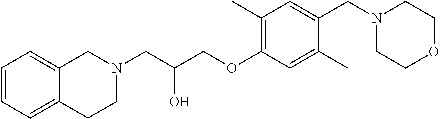 [SMILES: Cc1cc(OCC(O)CN2CCc3ccccc3C2)c(C)cc1CN1CCOCC1] | 410.2569 | 411.2 |
| D18. | 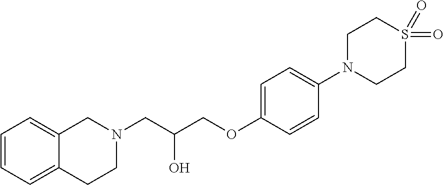 [SMILES: O=S1(=O)CCN(c2ccc(OCC(O)CN3CCc4ccccc4C3)cc2)CC1] | 416.177 | 416.9 |
| D19. | 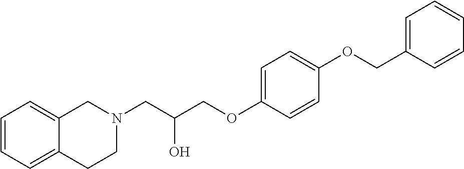 [SMILES: OC(COc1ccc(OCc2ccccc2)cc1)CN1CCc2ccccc2C1] | 389.1991 | 390.0 |
| D20. | 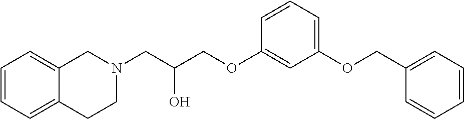 [SMILES: OC(COc1cccc(OCc2ccccc2)c1)CN1CCc2ccccc2C1] | 389.1991 | 390.1 |
| D21. | 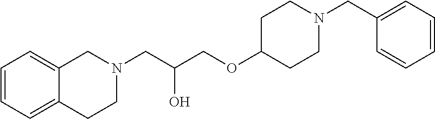 [SMILES: OC(COC1CCN(Cc2ccccc2)CC1)CN1CCc2ccccc2C1] | 380.2464 | 381.1 |
| D22. | 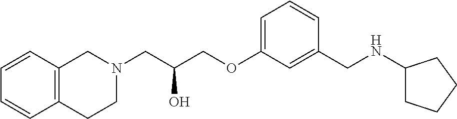 [SMILES: O[C@H](COc1cccc(CNC2CCCC2)c1)CN1CCc2ccccc2C1] | 380.2464 | 381.1 |
| D23. | 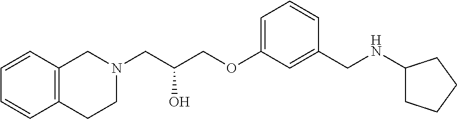 [SMILES: O[C@@H](COc1cccc(CNC2CCCC2)c1)CN1CCc2ccccc2C1] | 380.2464 | 381.1 |
| D24. | 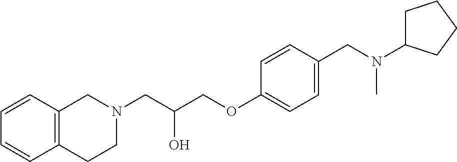 [SMILES: CN(Cc1ccc(OCC(O)CN2CCc3ccccc3C2)cc1)C1CCCC1] | 394.262 | 395.1 |
| D25. | 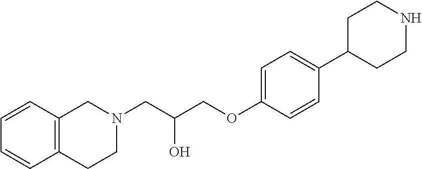 [SMILES: OC(COc1ccc(C2CCNCC2)cc1)CN1CCc2ccccc2C1] | 366.2307 | 367.2 |
| D26. | 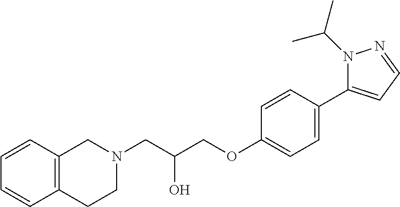 [SMILES: CC(C)n1nccc1-c1ccc(OCC(O)CN2CCc3ccccc3C2)cc1] | 391.226 | 392.0 |
| D27. | 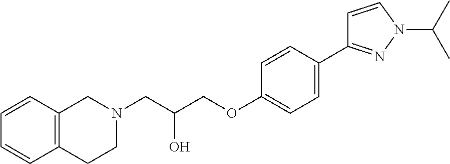 [SMILES: CC(C)n1ccc(-c2ccc(OCC(O)CN3CCc4ccccc4C3)cc2)n1] | 391.226 | 392.0 |
| D28. | 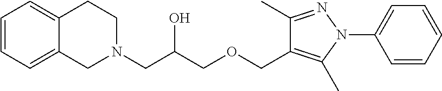 [SMILES: Cc1nn(-c2ccccc2)c(C)c1COCC(O)CN1CCc2ccccc2C1] | 391.226 | 391.9 |
| D29. | 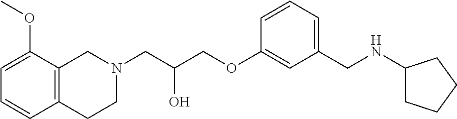 [SMILES: COc1cccc2c1CN(CC(O)COc1cccc(CNC3CCCC3)c1)CC2] | 410.2569 | 411.1 |
| D30. | 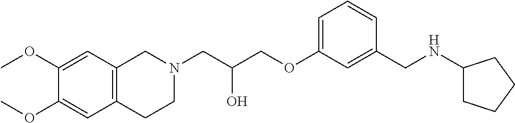 [SMILES: COc1cc2c(cc1OC)CN(CC(O)COc1cccc(CNC3CCCC3)c1)CC2] | 440.2675 | 441.0 |
| D31. | 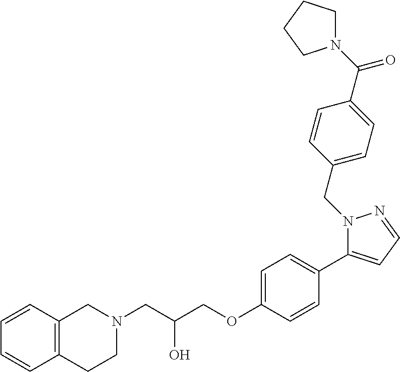 [SMILES: O=C(c1ccc(Cn2nccc2-c2ccc(OCC(O)CN3CCc4ccccc4C3)cc2)cc1)N1CCCC1] | 536.2787 | 537.3 |
| D32. | 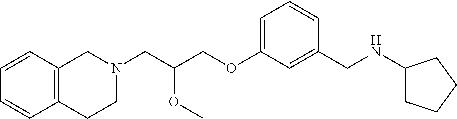 [SMILES: COC(COc1cccc(CNC2CCCC2)c1)CN1CCc2ccccc2C1] | 394.262 | 395.2 |
| D33. | 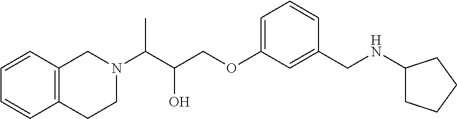 [SMILES: CC(C(O)COc1cccc(CNC2CCCC2)c1)N1CCc2ccccc2C1] | 394.262 | 395.2 |
| D34. | 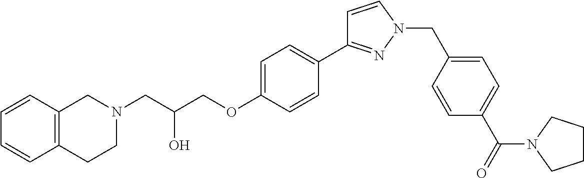 [SMILES: O=C(c1ccc(Cn2ccc(-c3ccc(OCC(O)CN4CCc5ccccc5C4)cc3)n2)cc1)N1CCCC1] | 536.2787 | 537.2 |
| D35. | 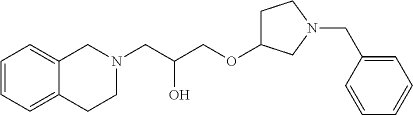 [SMILES: OC(COC1CCN(Cc2ccccc2)C1)CN1CCc2ccccc2C1] | 366.2307 | 367.1 |
| D36. | 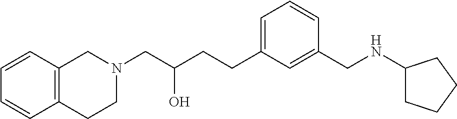 [SMILES: OC(CCc1cccc(CNC2CCCC2)c1)CN1CCc2ccccc2C1] | 378.2671 | 378.9 |
| D37. | 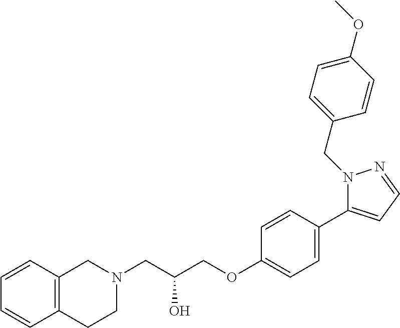 [SMILES: COc1ccc(Cn2nccc2-c2ccc(OC[C@H](O)CN3CCc4ccccc4C3)cc2)cc1] | 469.2365 | 469.9 |
| D38. | 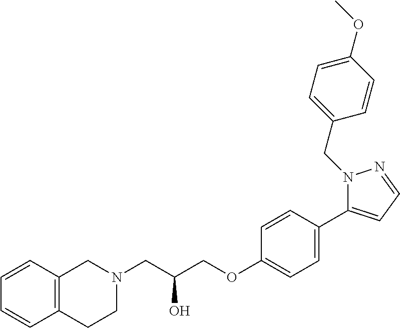 [SMILES: COc1ccc(Cn2nccc2-c2ccc(OC[C@@H](O)CN3CCc4ccccc4C3)cc2)cc1] | 469.2365 | 469.9 |
| D39. | 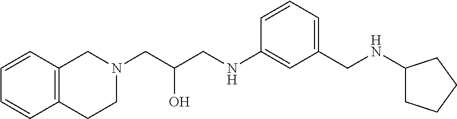 [SMILES: OC(CNc1cccc(CNC2CCCC2)c1)CN1CCc2ccccc2C1] | 379.2624 | 380.3 |
| D40. | 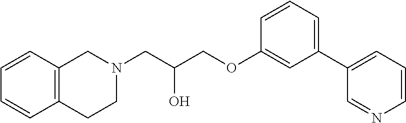 [SMILES: OC(COc1cccc(-c2cccnc2)c1)CN1CCc2ccccc2C1] | 360.1838 | 361.2 |
| D41. | 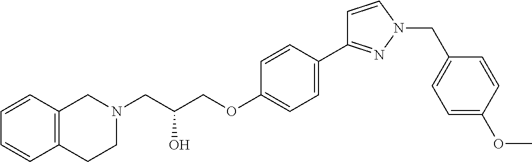 [SMILES: COc1ccc(Cn2ccc(-c3ccc(OC[C@H](O)CN4CCc5ccccc5C4)cc3)n2)cc1] | 469.2365 | 469.9 |
| D42. | 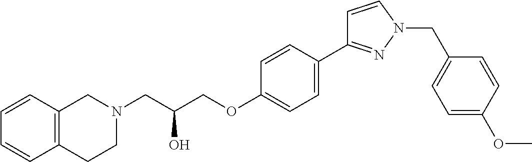 [SMILES: COc1ccc(Cn2ccc(-c3ccc(OC[C@@H](O)CN4CCc5ccccc5C4)cc3)n2)cc1] | 469.2365 | 470.2 |
| D43. | 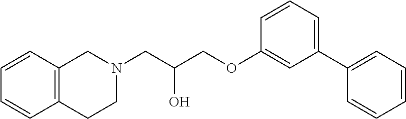 [SMILES: OC(COc1cccc(-c2ccccc2)c1)CN1CCc2ccccc2C1] | 359.1885 | 360.1 |
| D44. | 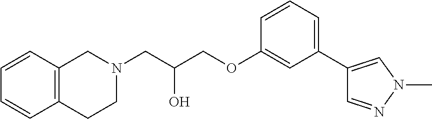 [SMILES: Cn1cc(-c2cccc(OCC(O)CN3CCc4ccccc4C3)c2)cn1] | 363.1947 | 364.2 |
| D45. | 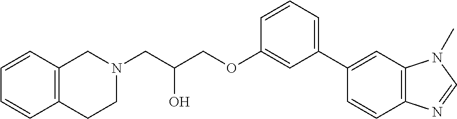 [SMILES: Cn1cnc2ccc(-c3cccc(OCC(O)CN4CCc5ccccc5C4)c3)cc21] | 413.2103 | 414.1 |
| D46. | 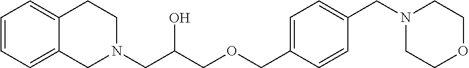 [SMILES: OC(COCc1ccc(CN2CCOCC2)cc1)CN1CCc2ccccc2C1] | 396.2413 | 397.0 |
| D47. | 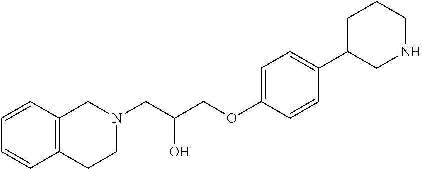 [SMILES: OC(COc1ccc(C2CCCNC2)cc1)CN1CCc2ccccc2C1] | 366.2307 | 367.2 |
| D48. | 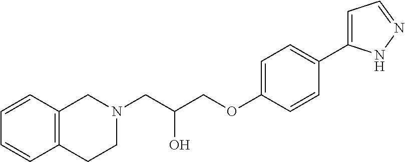 [SMILES: OC(COc1ccc(-c2ccn[nH]2)cc1)CN1CCc2ccccc2C1] | 349.179 | 350.1 |
| D49. | 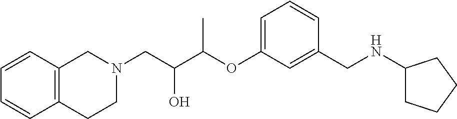 [SMILES: CC(Oc1cccc(CNC2CCCC2)c1)C(O)CN1CCc2ccccc2C1] | 394.262 | 395.1 |
| D50. | 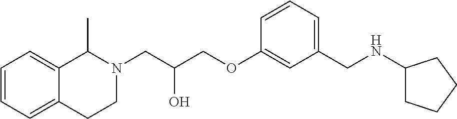 [SMILES: CC1c2ccccc2CCN1CC(O)COc1cccc(CNC2CCCC2)c1] | 394.262 | 395.2 |
| D51. | 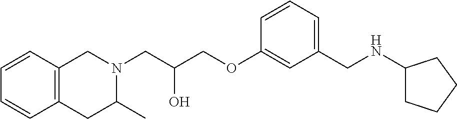 [SMILES: CC1Cc2ccccc2CN1CC(O)COc1cccc(CNC2CCCC2)c1] | 394.262 | 395.2 |
| D52. | 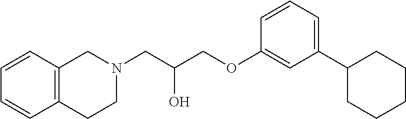 [SMILES: OC(COc1cccc(C2CCCCC2)c1)CN1CCc2ccccc2C1] | 365.2355 | 366.2 |
| D53. | 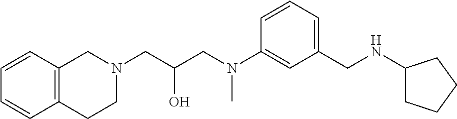 [SMILES: CN(CC(O)CN1CCc2ccccc2C1)c1cccc(CNC2CCCC2)c1] | 393.278 | 394.3 |
| D54. | 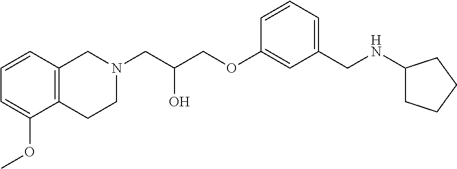 [SMILES: COc1cccc2c1CCN(CC(O)COc1cccc(CNC3CCCC3)c1)C2] | 410.2569 | 411.3 |
| D55. | 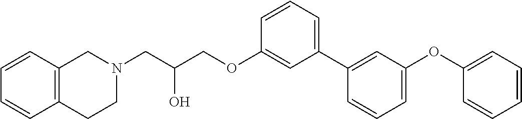 [SMILES: OC(COc1cccc(-c2cccc(Oc3ccccc3)c2)c1)CN1CCc2ccccc2C1] | 451.2147 | 452.3 |
| D56. | 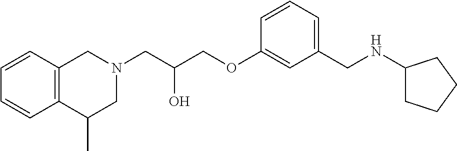 [SMILES: CC1CN(CC(O)COc2cccc(CNC3CCCC3)c2)Cc2ccccc21] | 394.262 | 395.2 |
| D57. | 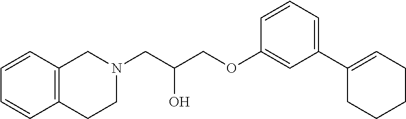 [SMILES: OC(COc1cccc(C2=CCCCC2)c1)CN1CCc2ccccc2C1] | 363.2198 | 364.3 |
| D58. | 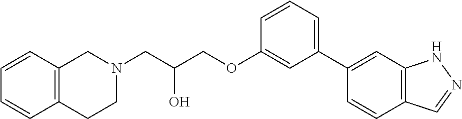 [SMILES: OC(COc1cccc(-c2ccc3cn[nH]c3c2)c1)CN1CCc2ccccc2C1] | 399.1947 | 400.2 |
| D59. | 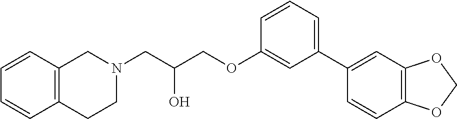 [SMILES: OC(COc1cccc(-c2ccc3c(c2)OCO3)c1)CN1CCc2ccccc2C1] | 403.1784 | 404.2 |
| D60. | 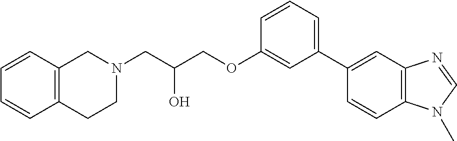 [SMILES: Cn1cnc2cc(-c3cccc(OCC(O)CN4CCc5ccccc5C4)c3)ccc21] | 413.2103 | 414.2 |
| D61. | 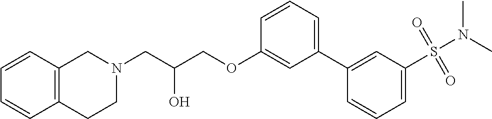 [SMILES: CN(C)S(=O)(=O)c1cccc(-c2cccc(OCC(O)CN3CCc4ccccc4C3)c2)c1] | 466.1926 | 467.2 |
| D62. | 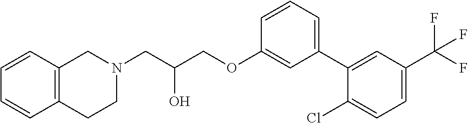 [SMILES: OC(COc1cccc(-c2cc(C(F)(F)F)ccc2Cl)c1)CN1CCc2ccccc2C1] | 461.1369 | 462.1 |
| D63. | 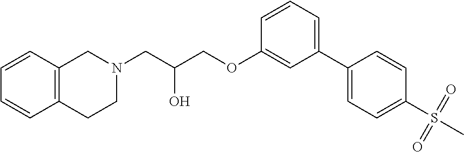 [SMILES: CS(=O)(=O)c1ccc(-c2cccc(OCC(O)CN3CCc4ccccc4C3)c2)cc1] | 437.1661 | 438.2 |
| D64. | 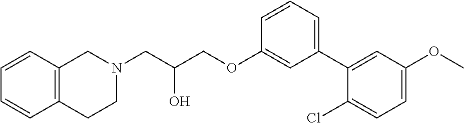 [SMILES: COc1ccc(Cl)c(-c2cccc(OCC(O)CN3CCc4ccccc4C3)c2)c1] | 423.1601 | 424.2 |
| D65. | 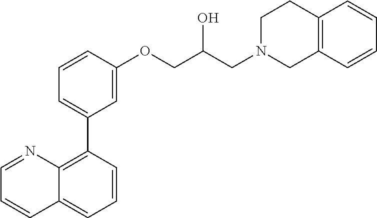 [SMILES: OC(COc1cccc(-c2cccc3cccnc23)c1)CN1CCc2ccccc2C1] | 410.1994 | 411.2 |
| D66. | 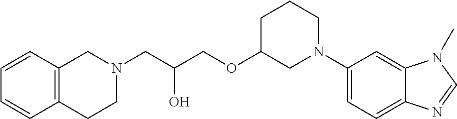 [SMILES: Cn1cnc2ccc(N3CCCC(OCC(O)CN4CCc5ccccc5C4)C3)cc21] | 420.2525 | 421.2 |
| D67. | 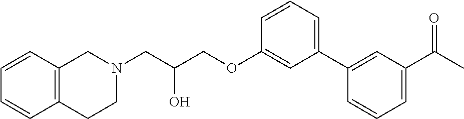 [SMILES: CC(=O)c1cccc(-c2cccc(OCC(O)CN3CCc4ccccc4C3)c2)c1] | 401.1991 | 402.2 |
| D68. | 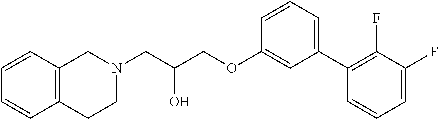 [SMILES: OC(COc1cccc(-c2cccc(F)c2F)c1)CN1CCc2ccccc2C1] | 395.1697 | 396.2 |
| D69. | 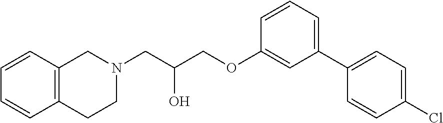 [SMILES: OC(COc1cccc(-c2ccc(Cl)cc2)c1)CN1CCc2ccccc2C1] | 393.1496 | 394.1 |
| D70. | 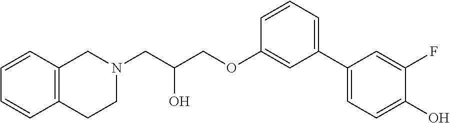 [SMILES: Oc1ccc(-c2cccc(OCC(O)CN3CCc4ccccc4C3)c2)cc1F] | 393.174 | 394.2 |
| D71. | 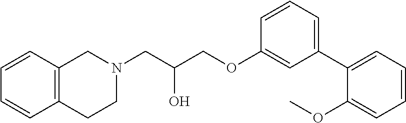 [SMILES: COc1ccccc1-c1cccc(OCC(O)CN2CCc3ccccc3C2)c1] | 389.1991 | 390.2 |
| D72. | 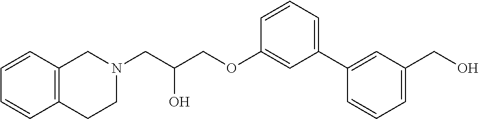 [SMILES: OCc1cccc(-c2cccc(OCC(O)CN3CCc4ccccc4C3)c2)c1] | 389.1991 | 390.2 |
| D73. | 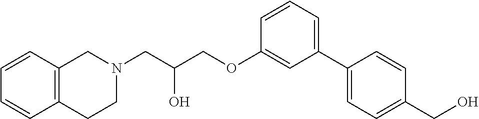 [SMILES: OCc1ccc(-c2cccc(OCC(O)CN3CCc4ccccc4C3)c2)cc1] | 389.1991 | 390.2 |
| D74. | 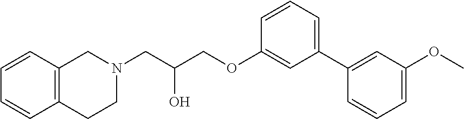 [SMILES: COc1cccc(-c2cccc(OCC(O)CN3CCc4ccccc4C3)c2)c1] | 389.1991 | 390.2 |
| D75. | 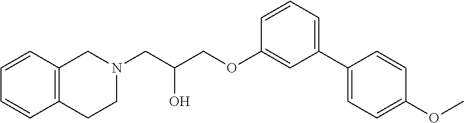 [SMILES: COc1ccc(-c2cccc(OCC(O)CN3CCc4ccccc4C3)c2)cc1] | 389.1991 | 390.2 |
| D76. | 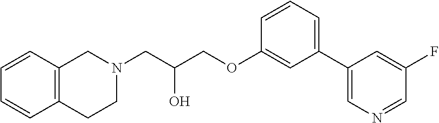 [SMILES: OC(COc1cccc(-c2cncc(F)c2)c1)CN1CCc2ccccc2C1] | 378.1744 | 379.2 |
| D77. | 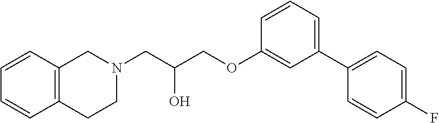 [SMILES: OC(COc1cccc(-c2ccc(F)cc2)c1)CN1CCc2ccccc2C1] | 377.1791 | 378.2 |
| D78. | 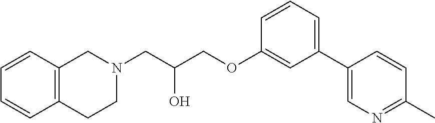 [SMILES: Cc1ccc(-c2cccc(OCC(O)CN3CCc4ccccc4C3)c2)cn1] | 374.1994 | 375.2 |
| D79. | 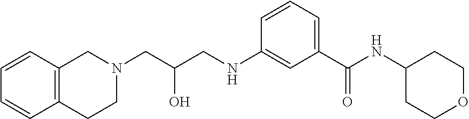 [SMILES: O=C(NC1CCOCC1)c1cccc(NCC(O)CN2CCc3ccccc3C2)c1] | 409.2365 | 410.2 |
| D80. | 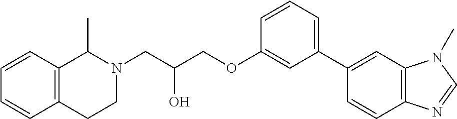 [SMILES: CC1c2ccccc2CCN1CC(O)COc1cccc(-c2ccc3ncn(C)c3c2)c1] | 427.226 | 428.2 |
| D81. | 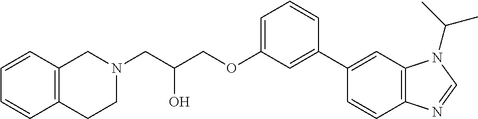 [SMILES: CC(C)n1cnc2ccc(-c3cccc(OCC(O)CN4CCc5ccccc5C4)c3)cc21] | 441.2416 | 442.3 |
| D82. | 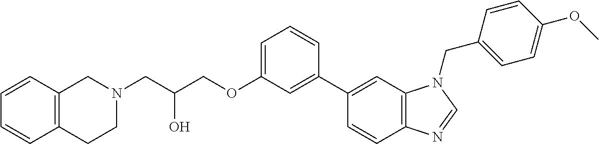 [SMILES: COc1ccc(Cn2cnc3ccc(-c4cccc(OCC(O)CN5CCc6ccccc6C5)c4)cc32)cc1] | 519.2522 | 520.3 |
| D83. | 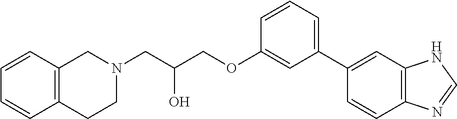 [SMILES: OC(COc1cccc(-c2ccc3nc[nH]c3c2)c1)CN1CCc2ccccc2C1] | 399.1947 | 400.2 |
| D84. | 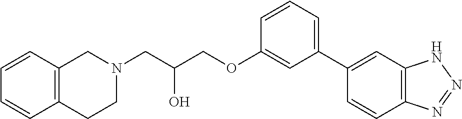 [SMILES: OC(COc1cccc(-c2ccc3nn[nH]c3c2)c1)CN1CCc2ccccc2C1] | 400.1899 | 401.2 |
| D85. | 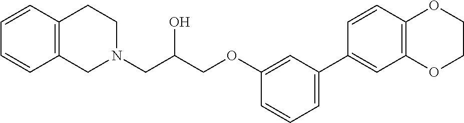 [SMILES: OC(COc1cccc(-c2ccc3c(c2)OCCO3)c1)CN1CCc2ccccc2C1] | 417.194 | 418.2 |
| D86. | 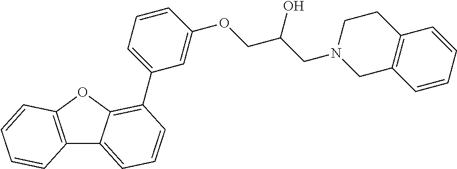 [SMILES: OC(COc1cccc(-c2cccc3c2oc2ccccc23)c1)CN1CCc2ccccc2C1] | 449.1991 | 450.2 |
| D87. | 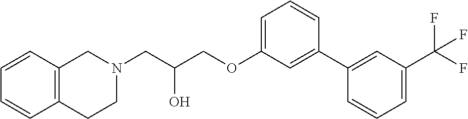 [SMILES: OC(COc1cccc(-c2cccc(C(F)(F)F)c2)c1)CN1CCc2ccccc2C1] | 427.1759 | 428.2 |
| D88. | 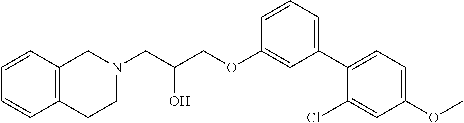 [SMILES: COc1ccc(-c2cccc(OCC(O)CN3CCc4ccccc4C3)c2)c(Cl)c1] | 423.1601 | 424.1 |
| D89. | 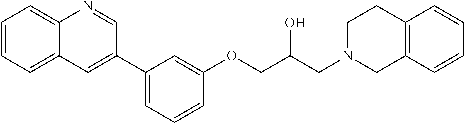 [SMILES: OC(COc1cccc(-c2cnc3ccccc3c2)c1)CN1CCc2ccccc2C1] | 410.1994 | 411.2 |
| D90. | 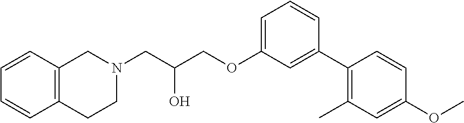 [SMILES: COc1ccc(-c2cccc(OCC(O)CN3CCc4ccccc4C3)c2)c(C)c1] | 403.2147 | 404.2 |
| D91. | 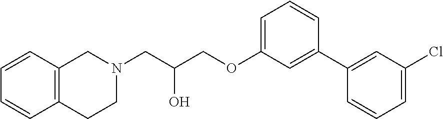 [SMILES: OC(COc1cccc(-c2cccc(Cl)c2)c1)CN1CCc2ccccc2C1] | 393.1496 | 394.2 |
| D92. | 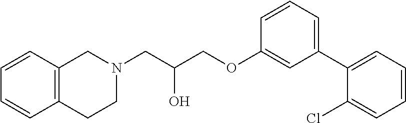 [SMILES: OC(COc1cccc(-c2ccccc2Cl)c1)CN1CCc2ccccc2C1] | 393.1496 | 394.2 |
| D93. | 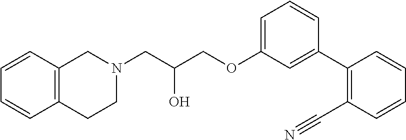 [SMILES: N#Cc1ccccc1-c1cccc(OCC(O)CN2CCc3ccccc3C2)c1] | 384.1838 | 385.2 |
| D94. | 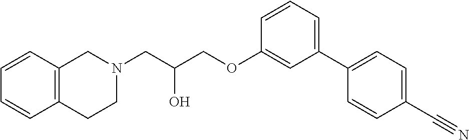 [SMILES: N#Cc1ccc(-c2cccc(OCC(O)CN3CCc4ccccc4C3)c2)cc1] | 384.1838 | 385.2 |
| D95. | 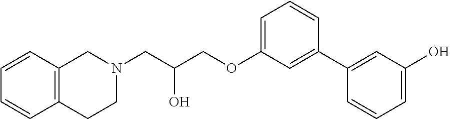 [SMILES: Oc1cccc(-c2cccc(OCC(O)CN3CCc4ccccc4C3)c2)c1] | 375.1834 | 376.2 |
| D96. | 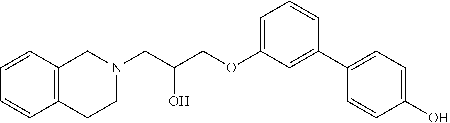 [SMILES: Oc1ccc(-c2cccc(OCC(O)CN3CCc4ccccc4C3)c2)cc1] | 375.1834 | 376.2 |
| D97. | 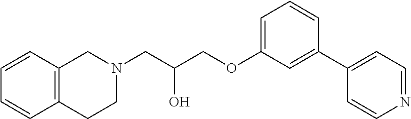 [SMILES: OC(COc1cccc(-c2ccncc2)c1)CN1CCc2ccccc2C1] | 360.1838 | 361.2 |
| D98. | 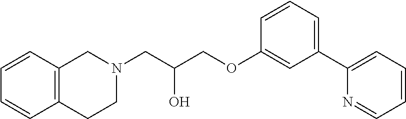 [SMILES: OC(COc1cccc(-c2ccccn2)c1)CN1CCc2ccccc2C1] | 360.1838 | 361.2 |
| D99. | 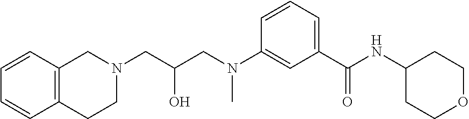 [SMILES: CN(CC(O)CN1CCc2ccccc2C1)c1cccc(C(=O)NC2CCOCC2)c1] | 423.2522 | 424.2 |
| D100. | 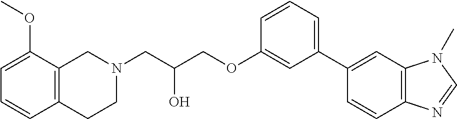 [SMILES: COc1cccc2c1CN(CC(O)COc1cccc(-c3ccc4ncn(C)c4c3)c1)CC2] | 443.2209 | 444.2 |
| D101. | 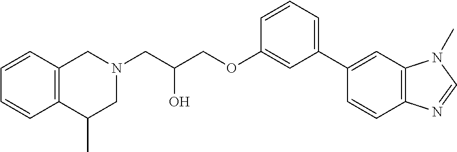 [SMILES: CC1CN(CC(O)COc2cccc(-c3ccc4ncn(C)c4c3)c2)Cc2ccccc21] | 427.226 | 428.2 |
| D102. | 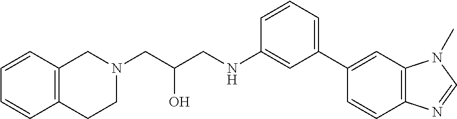 [SMILES: Cn1cnc2ccc(-c3cccc(NCC(O)CN4CCc5ccccc5C4)c3)cc21] | 412.2263 | 413.2 |
| D103. | 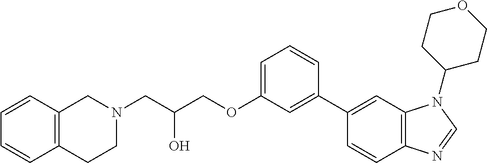 [SMILES: OC(COc1cccc(-c2ccc3ncn(C4CCOCC4)c3c2)c1)CN1CCc2ccccc2C1] | 483.2522 | 484.2 |
| D104. | 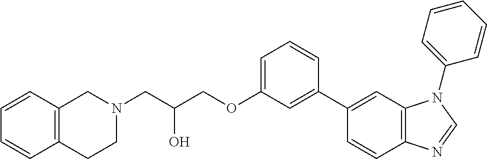 [SMILES: OC(COc1cccc(-c2ccc3ncn(-c4ccccc4)c3c2)c1)CN1CCc2ccccc2C1] | 475.226 | 476.2 |
| D105. | 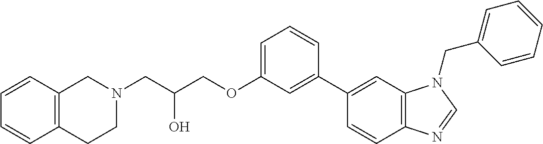 [SMILES: OC(COc1cccc(-c2ccc3ncn(Cc4ccccc4)c3c2)c1)CN1CCc2ccccc2C1] | 489.2416 | 490.2 |
| D106. | 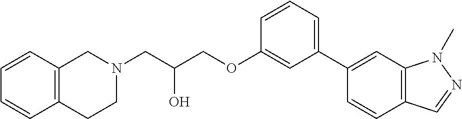 [SMILES: Cn1ncc2ccc(-c3cccc(OCC(O)CN4CCc5ccccc5C4)c3)cc21] | 413.2103 | 414.2 |
| D107. | 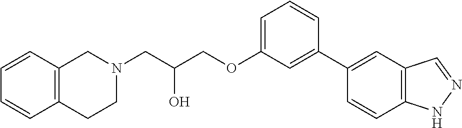 [SMILES: OC(COc1cccc(-c2ccc3[nH]ncc3c2)c1)CN1CCc2ccccc2C1] | 399.1947 | 400.1 |
| D108. | 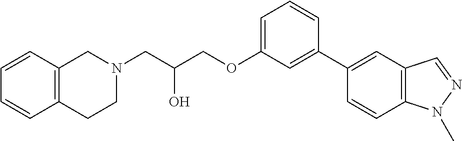 [SMILES: Cn1ncc2cc(-c3cccc(OCC(O)CN4CCc5ccccc5C4)c3)ccc21] | 413.2103 | 414.2 |
| D109. | 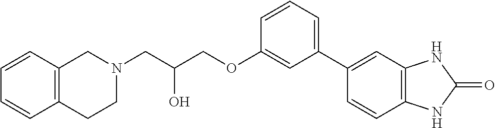 [SMILES: O=c1[nH]c2ccc(-c3cccc(OCC(O)CN4CCc5ccccc5C4)c3)cc2[nH]1] | 415.1896 | 416.2 |
| D110. | 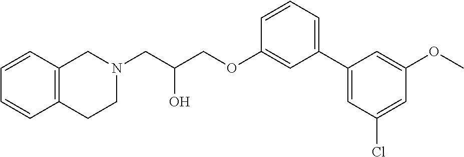 [SMILES: COc1cc(Cl)cc(-c2cccc(OCC(O)CN3CCc4ccccc4C3)c2)c1] | 423.1601 | 424.2 |
| D111. | 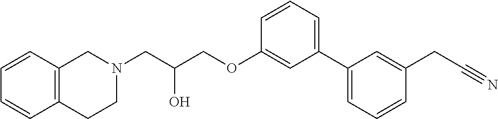 [SMILES: N#CCc1cccc(-c2cccc(OCC(O)CN3CCc4ccccc4C3)c2)c1] | 398.1994 | 399.2 |
| D112. | 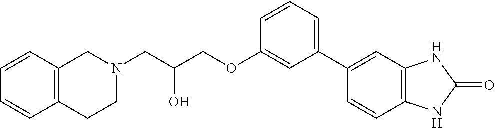 [SMILES: O=c1[nH]c2ccc(-c3cccc(OCC(O)CN4CCc5ccccc5C4)c3)cc2[nH]1] | 377.1791 | 378.2 |
| D113. | 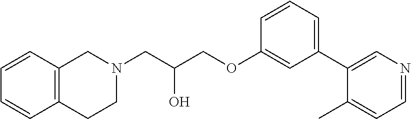 [SMILES: Cc1ccncc1-c1cccc(OCC(O)CN2CCc3ccccc3C2)c1] | 374.1994 | 375.2 |
| D114. | 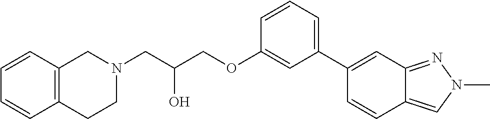 [SMILES: Cn1cc2ccc(-c3cccc(OCC(O)CN4CCc5ccccc5C4)c3)cc2n1] | 413.2103 | 414.2 |
| D115. | 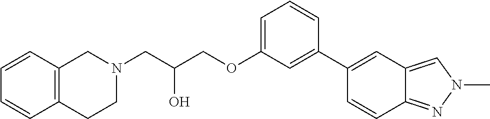 [SMILES: Cn1cc2cc(-c3cccc(OCC(O)CN4CCc5ccccc5C4)c3)ccc2n1] | 413.2103 | 414.2 |
| D116. | 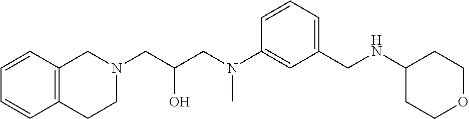 [SMILES: CN(CC(O)CN1CCc2ccccc2C1)c1cccc(CNC2CCOCC2)c1] | 409.2729 | 410.2 |
| D117. | 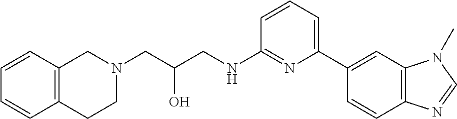 [SMILES: Cn1cnc2ccc(-c3cccc(NCC(O)CN4CCc5ccccc5C4)n3)cc21] | 413.2216 | 414.2 |
| D118. | 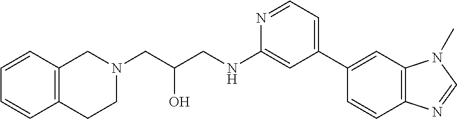 [SMILES: Cn1cnc2ccc(-c3ccnc(NCC(O)CN4CCc5ccccc5C4)c3)cc21] | 413.2216 | 414.2 |
| D119. | 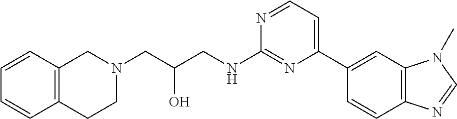 [SMILES: Cn1cnc2ccc(-c3ccnc(NCC(O)CN4CCc5ccccc5C4)n3)cc21] | 414.2168 | 415.2 |
| D120. | 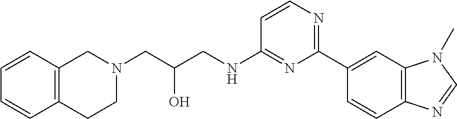 [SMILES: Cn1cnc2ccc(-c3nccc(NCC(O)CN4CCc5ccccc5C4)n3)cc21] | 414.2168 | 415.1 |
| D121. | 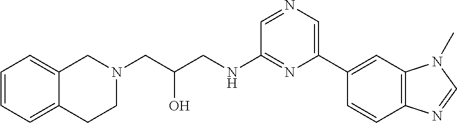 [SMILES: Cn1cnc2ccc(-c3cncc(NCC(O)CN4CCc5ccccc5C4)n3)cc21] | 414.2168 | 415.2 |
| D122. | 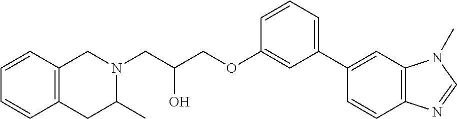 [SMILES: CC1Cc2ccccc2CN1CC(O)COc1cccc(-c2ccc3ncn(C)c3c2)c1] | 427.226 | 428.2 |
| D123. | 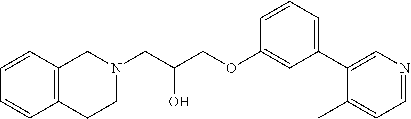 [SMILES: Cc1ccncc1-c1cccc(OCC(O)CN2CCc3ccccc3C2)c1] | 416.1558 | 417.2 |
| D124. | 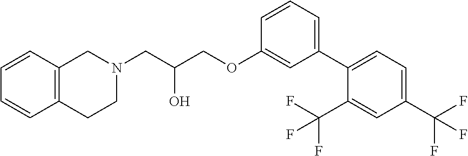 [SMILES: OC(COc1cccc(-c2ccc(C(F)(F)F)cc2C(F)(F)F)c1)CN1CCc2ccccc2C1] | 495.1633 | 496.2 |
| D125. | 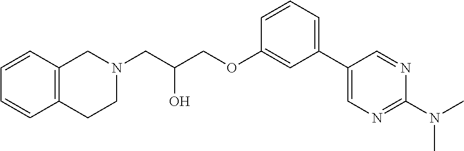 [SMILES: CN(C)c1ncc(-c2cccc(OCC(O)CN3CCc4ccccc4C3)c2)cn1] | 404.2212 | 405.2 |
| D126. | 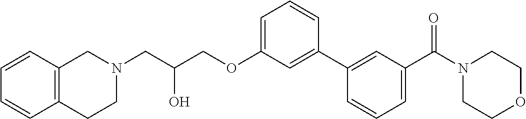 [SMILES: O=C(c1cccc(-c2cccc(OCC(O)CN3CCc4ccccc4C3)c2)c1)N1CCOCC1] | 472.2362 | 473.2 |
| D127. | 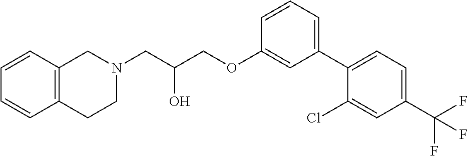 [SMILES: OC(COc1cccc(-c2ccc(C(F)(F)F)cc2Cl)c1)CN1CCc2ccccc2C1] | 461.1369 | 462.1 |
| D128. | 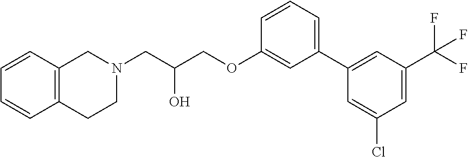 [SMILES: OC(COc1cccc(-c2cc(Cl)cc(C(F)(F)F)c2)c1)CN1CCc2ccccc2C1] | 461.1369 | 462.1 |
| D129. | 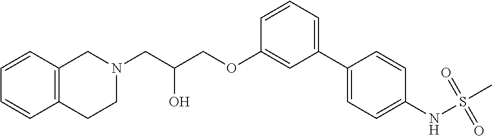 [SMILES: CS(=O)(=O)Nc1ccc(-c2cccc(OCC(O)CN3CCc4ccccc4C3)c2)cc1] | 452.177 | 453.2 |
| D130. | 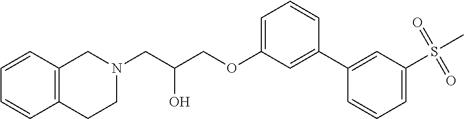 [SMILES: CS(=O)(=O)c1cccc(-c2cccc(OCC(O)CN3CCc4ccccc4C3)c2)c1] | 437.1661 | 438.2 |
| D131. | 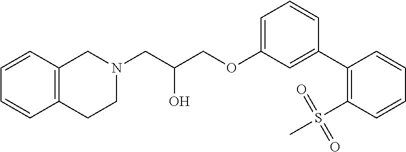 [SMILES: CS(=O)(=O)c1ccccc1-c1cccc(OCC(O)CN2CCc3ccccc3C2)c1] | 437.1661 | 438.2 |
| D132. | 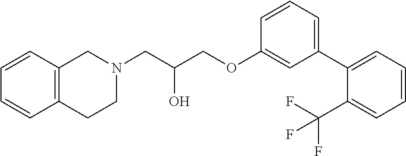 [SMILES: OC(COc1cccc(-c2ccccc2C(F)(F)F)c1)CN1CCc2ccccc2C1] | 427.1759 | 428.2 |
| D133. | 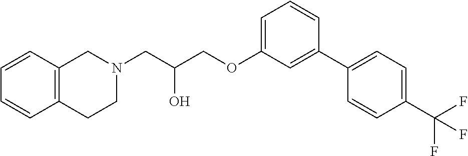 [SMILES: OC(COc1cccc(-c2ccc(C(F)(F)F)cc2)c1)CN1CCc2ccccc2C1] | 427.1759 | 428.2 |
| D134. | 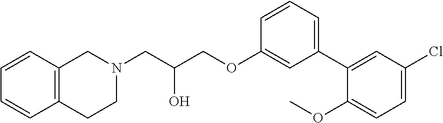 [SMILES: COc1ccc(Cl)cc1-c1cccc(OCC(O)CN2CCc3ccccc3C2)c1] | 423.1601 | 424.2 |
| D135. | 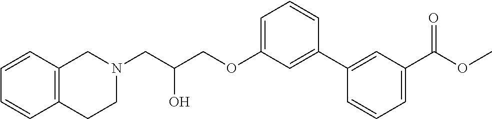 [SMILES: COC(=O)c1cccc(-c2cccc(OCC(O)CN3CCc4ccccc4C3)c2)c1] | 417.194 | 418.2 |
| D136. | 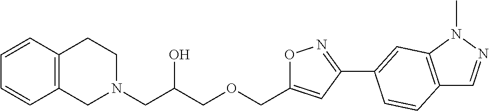 [SMILES: Cn1ncc2ccc(-c3cc(COCC(O)CN4CCc5ccccc5C4)on3)cc21] | 418.2005 | 419.2 |
| D137. | 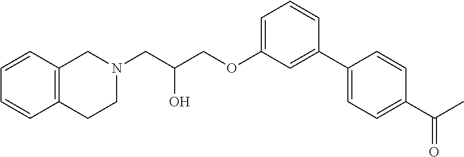 [SMILES: CC(=O)c1ccc(-c2cccc(OCC(O)CN3CCc4ccccc4C3)c2)cc1] | 401.1991 | 402.2 |
| D138. | 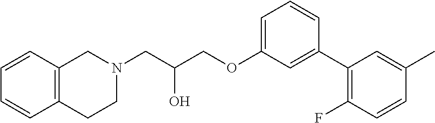 [SMILES: Cc1ccc(F)c(-c2cccc(OCC(O)CN3CCc4ccccc4C3)c2)c1] | 391.1948 | 392.2 |
| D139. | 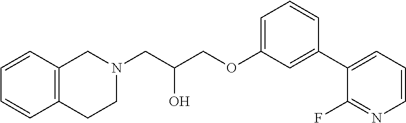 [SMILES: OC(COc1cccc(-c2cccnc2F)c1)CN1CCc2ccccc2C1] | 378.1744 | 379.2 |
| D140. | 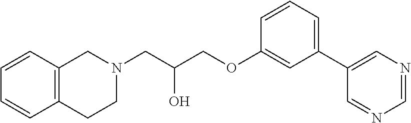 [SMILES: OC(COc1cccc(-c2cncnc2)c1)CN1CCc2ccccc2C1] | 361.179 | 362.2 |
| D141. | 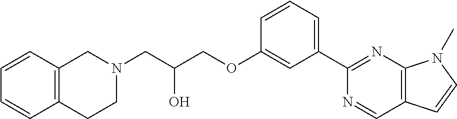 [SMILES: Cn1ccc2cnc(-c3cccc(OCC(O)CN4CCc5ccccc5C4)c3)nc21] | 414.2056 | 415.2 |
| D142. | 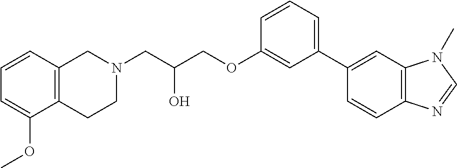 [SMILES: COc1cccc2c1CCN(CC(O)COc1cccc(-c3ccc4ncn(C)c4c3)c1)C2] | 443.2209 | 444.2 |
| D143. | 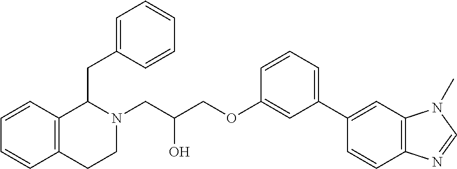 [SMILES: Cn1cnc2ccc(-c3cccc(OCC(O)CN4CCc5ccccc5C4Cc4ccccc4)c3)cc21] | 503.2573 | 504.2 |
| D144. | 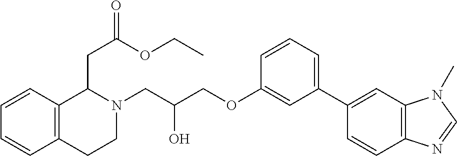 [SMILES: CCOC(=O)CC1c2ccccc2CCN1CC(O)COc1cccc(-c2ccc3ncn(C)c3c2)c1] | 499.2471 | 500.3 |
| D145. | 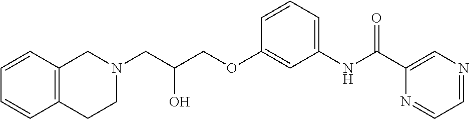 [SMILES: O=C(Nc1cccc(OCC(O)CN2CCc3ccccc3C2)c1)c1cnccn1] | 404.1848 | 405.2 |
| D146. | 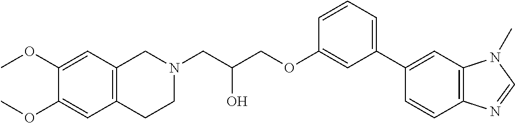 [SMILES: COc1cc2c(cc1OC)CN(CC(O)COc1cccc(-c3ccc4ncn(C)c4c3)c1)CC2] | 473.2315 | 474.3 |
| D147. | 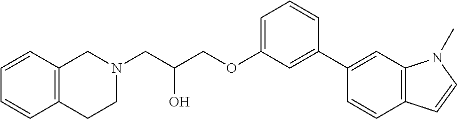 [SMILES: Cn1ccc2ccc(-c3cccc(OCC(O)CN4CCc5ccccc5C4)c3)cc21] | 412.2151 | 413.2 |
| D148. | 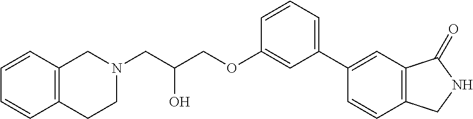 [SMILES: O=C1NCc2ccc(-c3cccc(OCC(O)CN4CCc5ccccc5C4)c3)cc21] | 414.1943 | 415.2 |
| D149. | 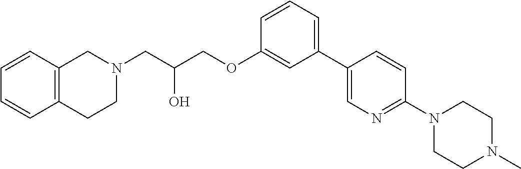 [SMILES: CN1CCN(c2ccc(-c3cccc(OCC(O)CN4CCc5ccccc5C4)c3)cn2)CC1] | 458.2682 | 459.2 |
| D150. | 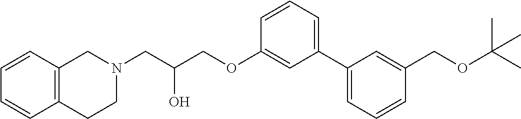 [SMILES: CC(C)(C)OCc1cccc(-c2cccc(OCC(O)CN3CCc4ccccc4C3)c2)c1] | 445.2617 | 446.3 |
| D151. | 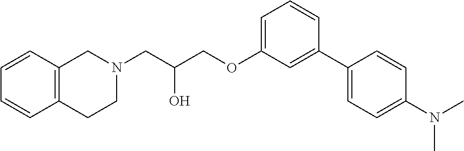 [SMILES: CN(C)c1ccc(-c2cccc(OCC(O)CN3CCc4ccccc4C3)c2)cc1] | 402.2307 | 403.3 |
| D152. | 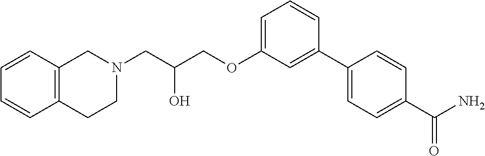 [SMILES: NC(=O)c1ccc(-c2cccc(OCC(O)CN3CCc4ccccc4C3)c2)cc1] | 402.1943 | 403.2 |
| D153. | 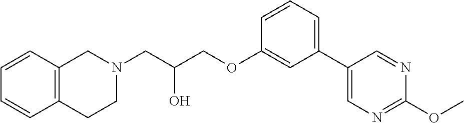 [SMILES: COc1ncc(-c2cccc(OCC(O)CN3CCc4ccccc4C3)c2)cn1] | 391.1896 | 392.2 |
| D154. | 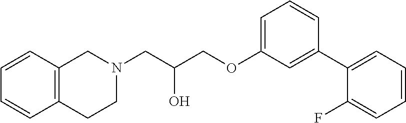 [SMILES: OC(COc1cccc(-c2ccccc2F)c1)CN1CCc2ccccc2C1] | 377.1791 | 378.2 |
| D155. | 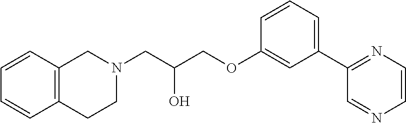 [SMILES: OC(COc1cccc(-c2cnccn2)c1)CN1CCc2ccccc2C1] | 361.179 | 362.2 |
| D156. | 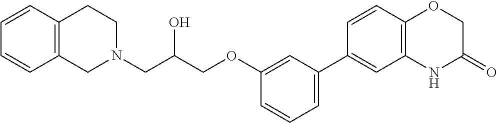 [SMILES: O=C1COc2ccc(-c3cccc(OCC(O)CN4CCc5ccccc5C4)c3)cc2N1] | 430.1893 | 431.2 |
| D157. | 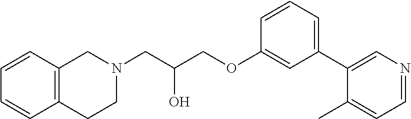 [SMILES: Cc1ccncc1-c1cccc(OCC(O)CN2CCc3ccccc3C2)c1] | 403.1896 | 404.2 |
| D158. | 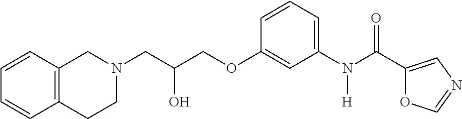 [SMILES: O=C(Nc1cccc(OCC(O)CN2CCc3ccccc3C2)c1)c1cnco1] | 393.1689 | 394.2 |
| D159. | 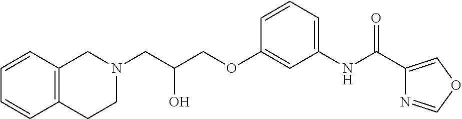 [SMILES: O=C(Nc1cccc(OCC(O)CN2CCc3ccccc3C2)c1)c1cocn1] | 393.1689 | 394.2 |
| D160. | 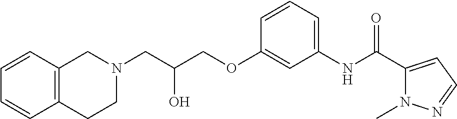 [SMILES: Cn1nccc1C(=O)Nc1cccc(OCC(O)CN2CCc3ccccc3C2)c1] | 406.2005 | 407.2 |
| D161. | 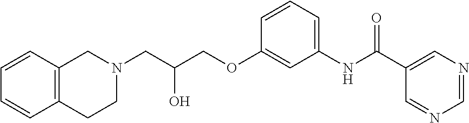 [SMILES: O=C(Nc1cccc(OCC(O)CN2CCc3ccccc3C2)c1)c1cncnc1] | 404.1848 | 405.2 |
| D162. | 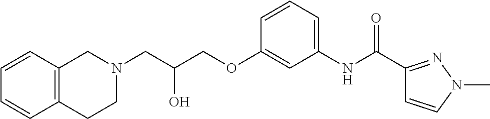 [SMILES: Cn1ccc(C(=O)Nc2cccc(OCC(O)CN3CCc4ccccc4C3)c2)n1] | 406.2005 | 407.2 |
| D163. | 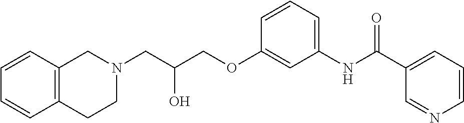 [SMILES: O=C(Nc1cccc(OCC(O)CN2CCc3ccccc3C2)c1)c1cccnc1] | 404.1848 | 405.1 |
| D164. | 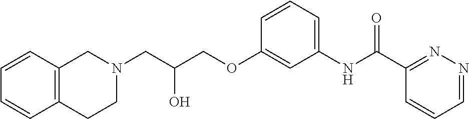 [SMILES: O=C(Nc1cccc(OCC(O)CN2CCc3ccccc3C2)c1)c1cccnn1] | 404.1848 | 405.2 |
| D165. | 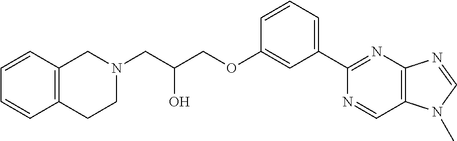 [SMILES: Cn1cnc2nc(-c3cccc(OCC(O)CN4CCc5ccccc5C4)c3)ncc21] | 415.2008 | 416.2 |
| D166. | 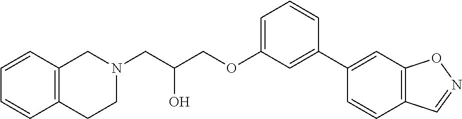 [SMILES: OC(COc1cccc(-c2ccc3cnoc3c2)c1)CN1CCc2ccccc2C1] | 400.1787 | 401.2 |
| D167. | 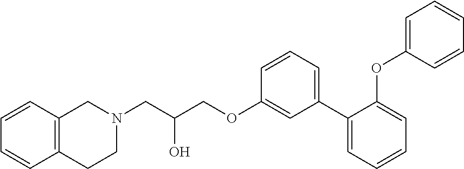 [SMILES: OC(COc1cccc(-c2ccccc2Oc2ccccc2)c1)CN1CCc2ccccc2C1] | 451.2147 | 452.3 |
| D168. | 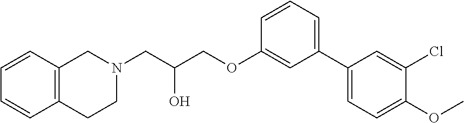 [SMILES: COc1ccc(-c2cccc(OCC(O)CN3CCc4ccccc4C3)c2)cc1Cl] | 423.1601 | 424.2 |
| D169. | 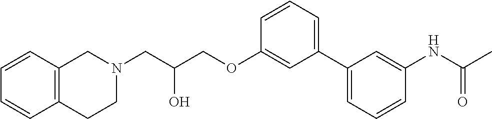 [SMILES: CC(=O)Nc1cccc(-c2cccc(OCC(O)CN3CCc4ccccc4C3)c2)c1] | 416.21 | 417.3 |
| D170. | 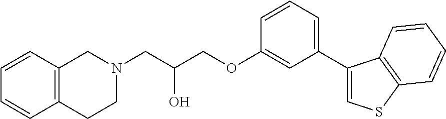 [SMILES: OC(COc1cccc(-c2csc3ccccc23)c1)CN1CCc2ccccc2C1] | 415.1606 | 416.2 |
| D171. | 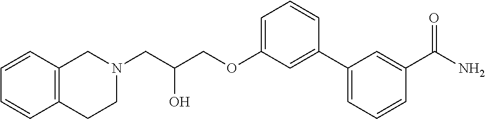 [SMILES: NC(=O)c1cccc(-c2cccc(OCC(O)CN3CCc4ccccc4C3)c2)c1] | 402.1943 | 403.2 |
| D172. | 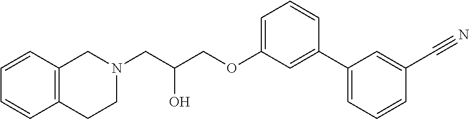 [SMILES: N#Cc1cccc(-c2cccc(OCC(O)CN3CCc4ccccc4C3)c2)c1] | 384.1838 | 385.2 |
| D173. | 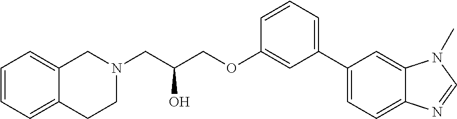 [SMILES: Cn1cnc2ccc(-c3cccc(OC[C@@H](O)CN4CCc5ccccc5C4)c3)cc21] | 413.2103 | 414.3 |
| D174. | 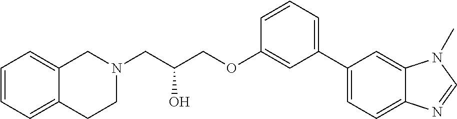 [SMILES: Cn1cnc2ccc(-c3cccc(OC[C@H](O)CN4CCc5ccccc5C4)c3)cc21] | 413.2103 | 414.3 |
| D175. | 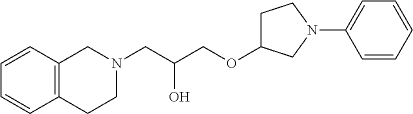 [SMILES: OC(COC1CCN(c2ccccc2)C1)CN1CCc2ccccc2C1] | 352.2151 | 353.2 |
| D176. | 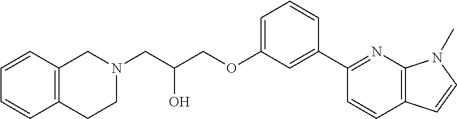 [SMILES: Cn1ccc2ccc(-c3cccc(OCC(O)CN4CCc5ccccc5C4)c3)nc21] | 413.2103 | 414.3 |
| D177. | 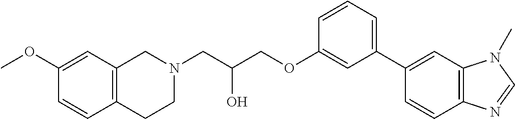 [SMILES: COc1ccc2c(c1)CN(CC(O)COc1cccc(-c3ccc4ncn(C)c4c3)c1)CC2] | 443.2209 | 444.3 |
| D178. | 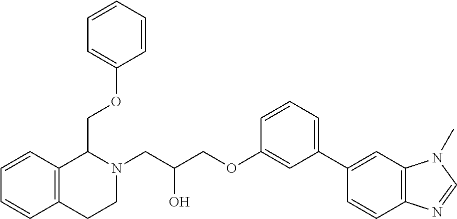 [SMILES: Cn1cnc2ccc(-c3cccc(OCC(O)CN4CCc5ccccc5C4COc4ccccc4)c3)cc21] | 519.2522 | 520.3 |
| D179. | 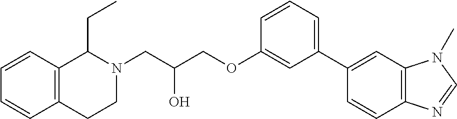 [SMILES: CCC1c2ccccc2CCN1CC(O)COc1cccc(-c2ccc3ncn(C)c3c2)c1] | 441.2416 | 442.2 |
| D180. | 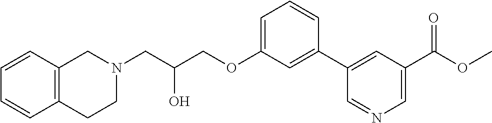 [SMILES: COC(=O)c1cncc(-c2cccc(OCC(O)CN3CCc4ccccc4C3)c2)c1] | 418.1893 | 419.2 |
| D181. | 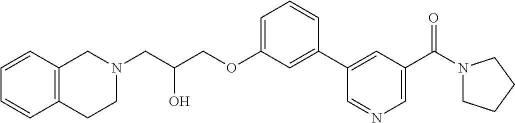 [SMILES: O=C(c1cncc(-c2cccc(OCC(O)CN3CCc4ccccc4C3)c2)c1)N1CCCC1] | 457.2365 | 458.2 |
| D182. | 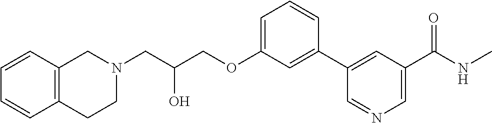 [SMILES: CNC(=O)c1cncc(-c2cccc(OCC(O)CN3CCc4ccccc4C3)c2)c1] | 417.2052 | 418.2 |
| D183. | 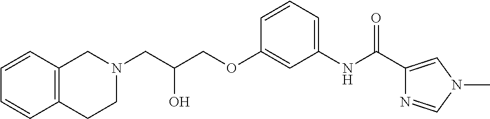 [SMILES: Cn1cnc(C(=O)Nc2cccc(OCC(O)CN3CCc4ccccc4C3)c2)c1] | 406.2005 | 407.2 |
| D184. | 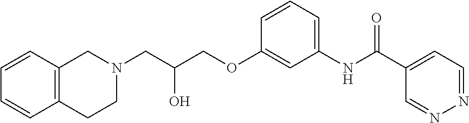 [SMILES: O=C(Nc1cccc(OCC(O)CN2CCc3ccccc3C2)c1)c1ccnnc1] | 404.1848 | 405.2 |
| D185. | 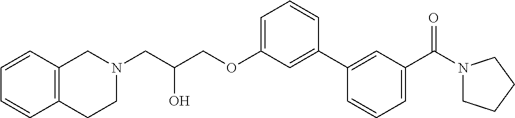 [SMILES: O=C(c1cccc(-c2cccc(OCC(O)CN3CCc4ccccc4C3)c2)c1)N1CCCC1] | 456.2413 | 457.2 |
| D186. | 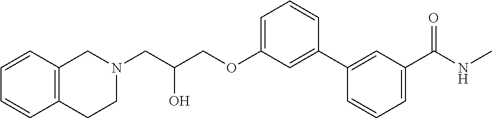 [SMILES: CNC(=O)c1cccc(-c2cccc(OCC(O)CN3CCc4ccccc4C3)c2)c1] | 416.21 | 417.2 |
| D187. | 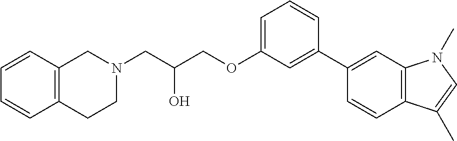 [SMILES: Cc1cn(C)c2cc(-c3cccc(OCC(O)CN4CCc5ccccc5C4)c3)ccc12] | 426.2307 | 427.3 |
| D188. | 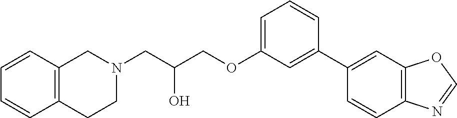 [SMILES: OC(COc1cccc(-c2ccc3ncoc3c2)c1)CN1CCc2ccccc2C1] | 400.1787 | 401.2 |
| D189. | 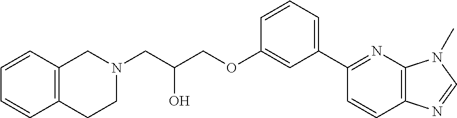 [SMILES: Cn1cnc2ccc(-c3cccc(OCC(O)CN4CCc5ccccc5C4)c3)nc21] | 414.2056 | 415.2 |
| D190. | 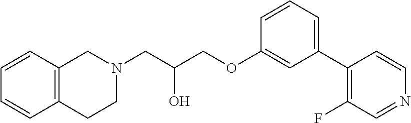 [SMILES: OC(COc1cccc(-c2ccncc2F)c1)CN1CCc2ccccc2C1] | 378.1744 | 379.2 |
| D191. | 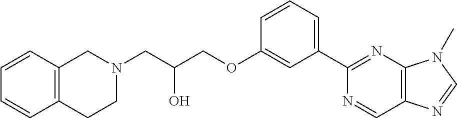 [SMILES: Cn1cnc2cnc(-c3cccc(OCC(O)CN4CCc5ccccc5C4)c3)nc21] | 415.2008 | 416.2 |
| D192. | 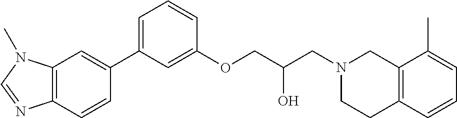 [SMILES: Cc1cccc2c1CN(CC(O)COc1cccc(-c3ccc4ncn(C)c4c3)c1)CC2] | 427.226 | 428.2 |
| D193. | 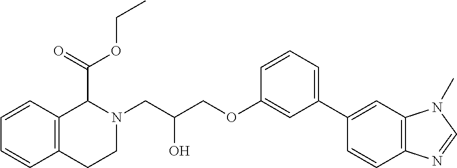 [SMILES: CCOC(=O)C1c2ccccc2CCN1CC(O)COc1cccc(-c2ccc3ncn(C)c3c2)c1] | 485.2315 | 486.3 |
| D194. | 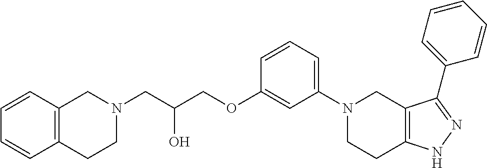 [SMILES: OC(COc1cccc(N2CCc3[nH]nc(-c4ccccc4)c3C2)c1)CN1CCc2ccccc2C1] | 480.2525 | 481.3 |
| D195. | 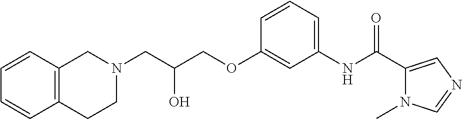 [SMILES: Cn1cncc1C(=O)Nc1cccc(OCC(O)CN2CCc3ccccc3C2)c1] | 406.2005 | 407.2 |
| D196. | 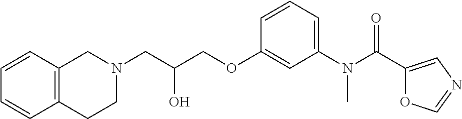 [SMILES: CN(C(=O)c1cnco1)c1cccc(OCC(O)CN2CCc3ccccc3C2)c1] | 407.1845 | 408.2 |
| D197. | 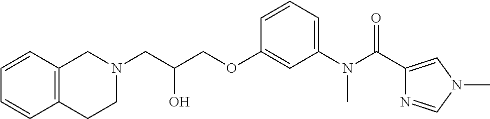 [SMILES: CN(C(=O)c1cn(C)cn1)c1cccc(OCC(O)CN2CCc3ccccc3C2)c1] | 420.2161 | 421.3 |
| D198. | 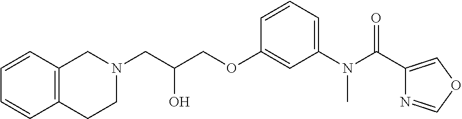 [SMILES: CN(C(=O)c1cocn1)c1cccc(OCC(O)CN2CCc3ccccc3C2)c1] | 407.1845 | 408.3 |
| D199. | 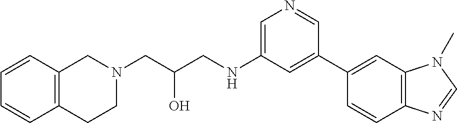 [SMILES: Cn1cnc2ccc(-c3cncc(NCC(O)CN4CCc5ccccc5C4)c3)cc21] | 413.2216 | 414.2 |
| D200. | 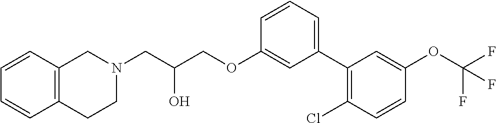 [SMILES: OC(COc1cccc(-c2cc(OC(F)(F)F)ccc2Cl)c1)CN1CCc2ccccc2C1] | 477.1319 | 478.2 |
| D201. | 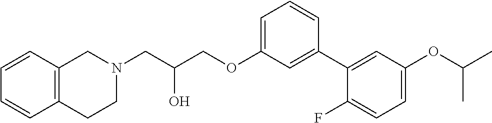 [SMILES: CC(C)Oc1ccc(F)c(-c2cccc(OCC(O)CN3CCc4ccccc4C3)c2)c1] | 435.221 | 436.2 |
| D202. | 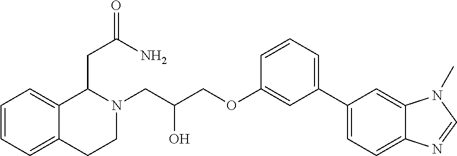 [SMILES: Cn1cnc2ccc(-c3cccc(OCC(O)CN4CCc5ccccc5C4CC(N)=O)c3)cc21] | 470.2318 | 471.2 |
| D203. | 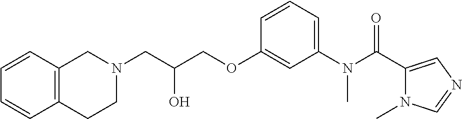 [SMILES: CN(C(=O)c1cncn1C)c1cccc(OCC(O)CN2CCc3ccccc3C2)c1] | 420.2161 | 421.2 |
| D204. | 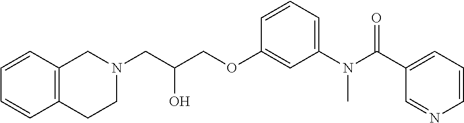 [SMILES: CN(C(=O)c1cccnc1)c1cccc(OCC(O)CN2CCc3ccccc3C2)c1] | 417.2052 | 418.2 |
| D205. | 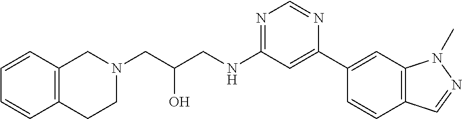 [SMILES: Cn1ncc2ccc(-c3cc(NCC(O)CN4CCc5ccccc5C4)ncn3)cc21] | 414.2168 | 415.2 |
| D206. | 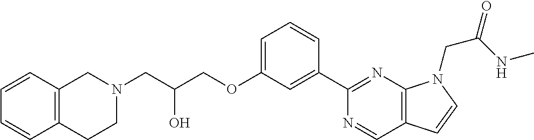 [SMILES: CNC(=O)Cn1ccc2cnc(-c3cccc(OCC(O)CN4CCc5ccccc5C4)c3)nc21] | 471.227 | 472.2 |
| D207. | 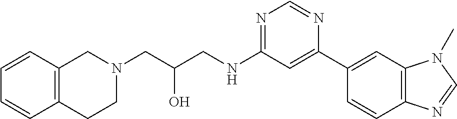 [SMILES: Cn1cnc2ccc(-c3cc(NCC(O)CN4CCc5ccccc5C4)ncn3)cc21] | 414.2168 | 415.2 |
| D208. | 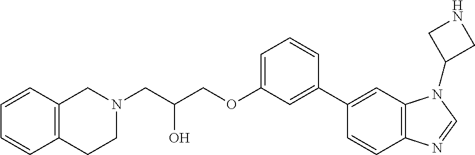 [SMILES: OC(COc1cccc(-c2ccc3ncn(C4CNC4)c3c2)c1)CN1CCc2ccccc2C1] | 454.2369 | 455.2 |
| D209. | 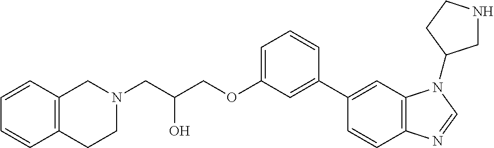 [SMILES: OC(COc1cccc(-c2ccc3ncn(C4CCNC4)c3c2)c1)CN1CCc2ccccc2C1] | 468.2525 | 469.3 |
| D210. | 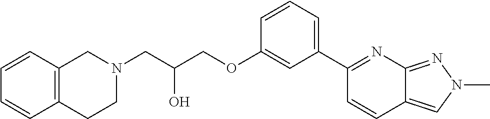 [SMILES: Cn1cc2ccc(-c3cccc(OCC(O)CN4CCc5ccccc5C4)c3)nc2n1] | 414.2056 | 415.2 |
| D211. | 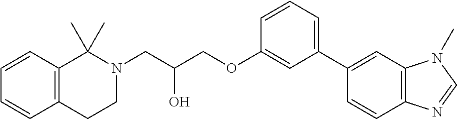 [SMILES: Cn1cnc2ccc(-c3cccc(OCC(O)CN4CCc5ccccc5C4(C)C)c3)cc21] | 441.2416 | 442.2 |
| D212. | 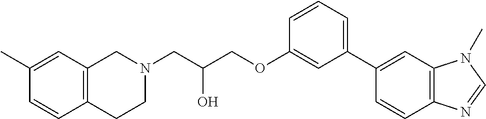 [SMILES: Cc1ccc2c(c1)CN(CC(O)COc1cccc(-c3ccc4ncn(C)c4c3)c1)CC2] | 427.226 | 428.2 |
| D213. | 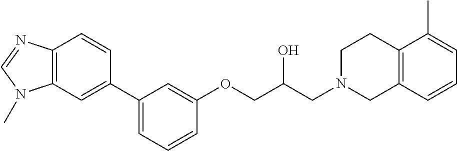 [SMILES: Cc1cccc2c1CCN(CC(O)COc1cccc(-c3ccc4ncn(C)c4c3)c1)C2] | 427.226 | 428.2 |
| D214. | 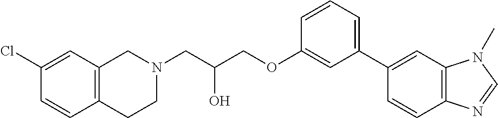 [SMILES: Cn1cnc2ccc(-c3cccc(OCC(O)CN4CCc5ccc(Cl)cc5C4)c3)cc21] | 447.1714 | 448.2 |
| D215. | 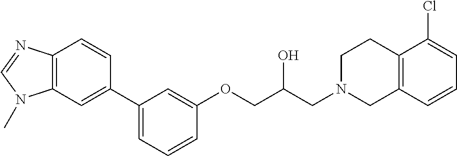 [SMILES: Cn1cnc2ccc(-c3cccc(OCC(O)CN4CCc5c(Cl)cccc5C4)c3)cc21] | 447.1714 | 448.1 |
| D216. | 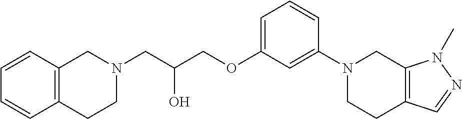 [SMILES: Cn1ncc2c1CN(c1cccc(OCC(O)CN3CCc4ccccc4C3)c1)CC2] | 418.2369 | 419.2 |
| D217. | 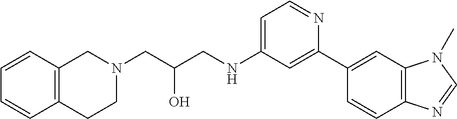 [SMILES: Cn1cnc2ccc(-c3cc(NCC(O)CN4CCc5ccccc5C4)ccn3)cc21] | 413.2216 | 414.2 |
| D218. | 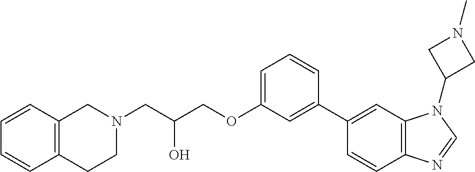 [SMILES: CN1CC(n2cnc3ccc(-c4cccc(OCC(O)CN5CCc6ccccc6C5)c4)cc32)C1] | 468.2525 | 469.3 |
| D219. | 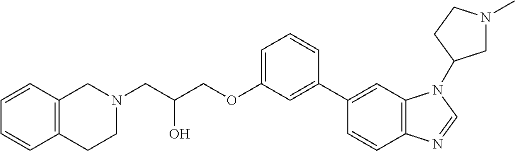 [SMILES: CN1CCC(n2cnc3ccc(-c4cccc(OCC(O)CN5CCc6ccccc6C5)c4)cc32)C1] | 482.2682 | 483.2 |
| D220. | 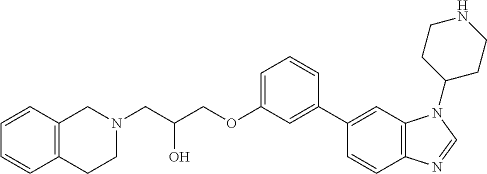 [SMILES: OC(COc1cccc(-c2ccc3ncn(C4CCNCC4)c3c2)c1)CN1CCc2ccccc2C1] | 482.2682 | 483.3 |
| D221. | 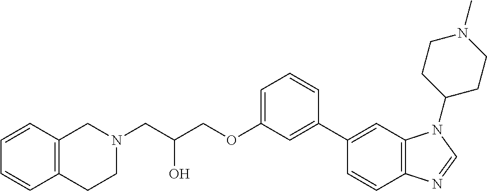 [SMILES: CN1CCC(n2cnc3ccc(-c4cccc(OCC(O)CN5CCc6ccccc6C5)c4)cc32)CC1] | 496.2838 | 497.3 |
| D222. | 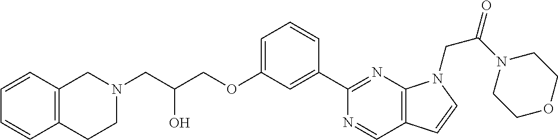 [SMILES: O=C(Cn1ccc2cnc(-c3cccc(OCC(O)CN4CCc5ccccc5C4)c3)nc21)N1CCOCC1] | 527.2533 | 528.2 |
| D223. | 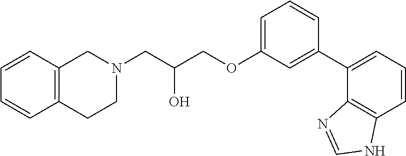 [SMILES: OC(COc1cccc(-c2cccc3[nH]cnc23)c1)CN1CCc2ccccc2C1] | 399.1947 | 400.1 |
| D224. | 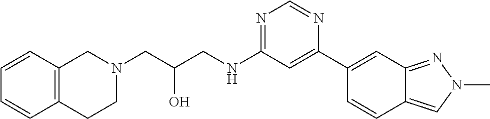 [SMILES: Cn1cc2ccc(-c3cc(NCC(O)CN4CCc5ccccc5C4)ncn3)cc2n1] | 414.2168 | 415.2 |
| D225. | 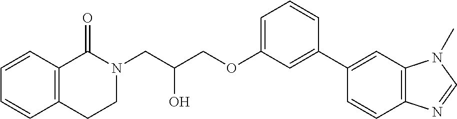 [SMILES: Cn1cnc2ccc(-c3cccc(OCC(O)CN4CCc5ccccc5C4=O)c3)cc21] | 427.1896 | 428.2 |
| D226. | 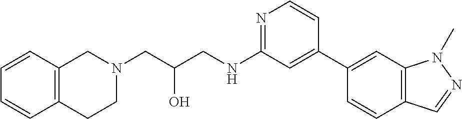 [SMILES: Cn1ncc2ccc(-c3ccnc(NCC(O)CN4CCc5ccccc5C4)c3)cc21] | 413.2216 | 414.2 |
| D227. | 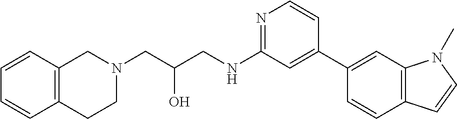 [SMILES: Cn1ccc2ccc(-c3ccnc(NCC(O)CN4CCc5ccccc5C4)c3)cc21] | 412.2263 | 413.2 |
| D228. | 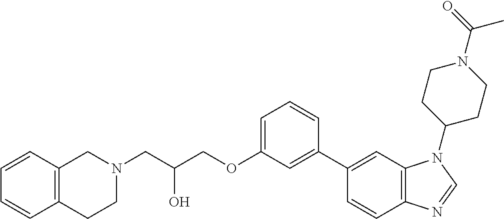 [SMILES: CC(=O)N1CCC(n2cnc3ccc(-c4cccc(OCC(O)CN5CCc6ccccc6C5)c4)cc32)CC1] | 524.2787 | 525.3 |
| D229. | 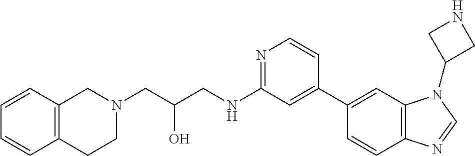 [SMILES: OC(CNc1cc(-c2ccc3ncn(C4CNC4)c3c2)ccn1)CN1CCc2ccccc2C1] | 454.2481 | 455.2 |
| D230. | 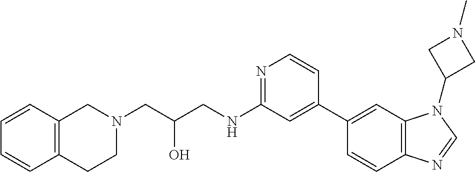 [SMILES: CN1CC(n2cnc3ccc(-c4ccnc(NCC(O)CN5CCc6ccccc6C5)c4)cc32)C1] | 468.2638 | 469.2 |
| D231. | 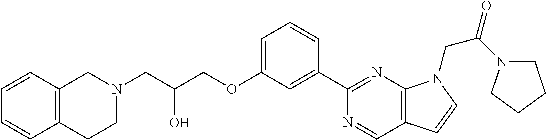 [SMILES: O=C(Cn1ccc2cnc(-c3cccc(OCC(O)CN4CCc5ccccc5C4)c3)nc21)N1CCCC1] | 511.2583 | 512.2 |
| D232. | 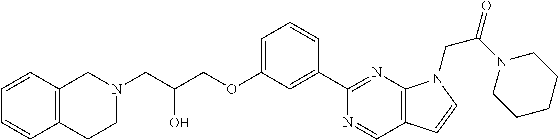 [SMILES: O=C(Cn1ccc2cnc(-c3cccc(OCC(O)CN4CCc5ccccc5C4)c3)nc21)N1CCCCC1] | 525.274 | 526.2 |
| D233. | 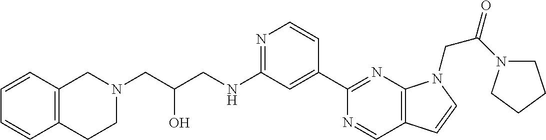 | 511.2696 | 512.2 |
| D234. | 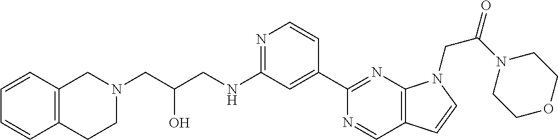 | 527.2645 | 528.2 |
| D235. | 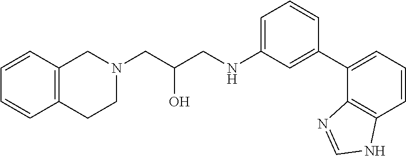 | 398.2107 | 399.2 |
| D236. | 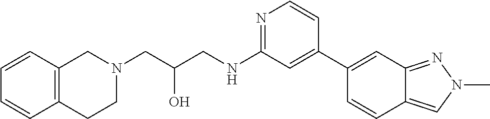 [SMILES: Cn1cc2ccc(-c3ccnc(NCC(O)CN4CCc5ccccc5C4)c3)cc2n1] | 413.2216 | 414.2 |
| D237. | 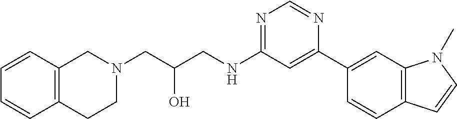 [SMILES: Cn1ccc2ccc(-c3cc(NCC(O)CN4CCc5ccccc5C4)ncn3)cc21] | 413.2216 | 414.2 |
| D238. | 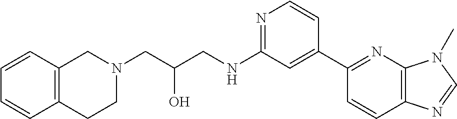 [SMILES: Cn1cnc2ccc(-c3ccnc(NCC(O)CN4CCc5ccccc5C4)c3)nc21] | 414.2168 | 415.2 |
| D239. | 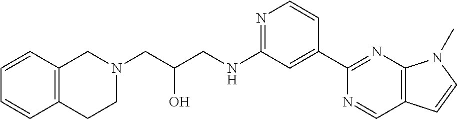 [SMILES: Cn1ccc2cnc(-c3ccnc(NCC(O)CN4CCc5ccccc5C4)c3)nc21] | 414.2168 | 415.2 |
| D240. | 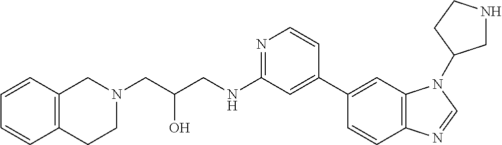 [SMILES: OC(CNc1cc(-c2ccc3ncn(C4CCNC4)c3c2)ccn1)CN1CCc2ccccc2C1] | 468.2638 | 469.2 |
| D241. | 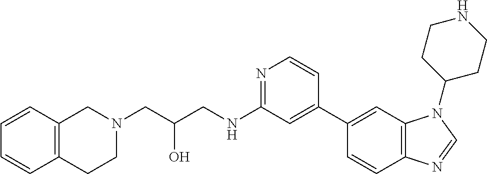 [SMILES: OC(CNc1cc(-c2ccc3ncn(C4CCNCC4)c3c2)ccn1)CN1CCc2ccccc2C1] | 482.2794 | 483.3 |
| D242. | 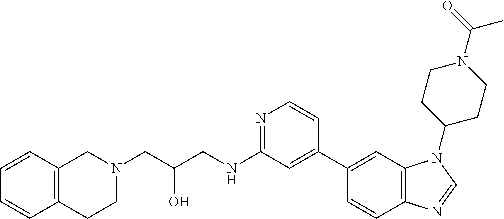 [SMILES: CC(=O)N1CCC(n2cnc3ccc(-c4ccnc(NCC(O)CN5CCc6ccccc6C5)c4)cc32)CC1] | 524.29 | 525.4 |
| D243. | 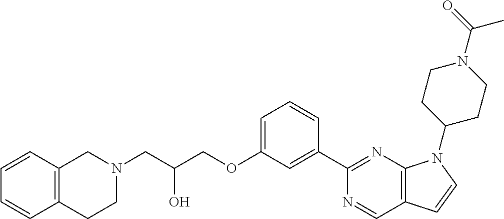 [SMILES: CC(=O)N1CCC(n2ccc3cnc(-c4cccc(OCC(O)CN5CCc6ccccc6C5)c4)nc32)CC1] | 525.274 | 526.2 |
| D244. | 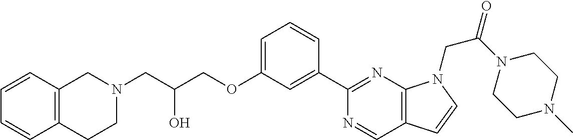 [SMILES: CN1CCN(C(=O)Cn2ccc3cnc(-c4cccc(OCC(O)CN5CCc6ccccc6C5)c4)nc32)CC1] | 540.2849 | 541.2 |
| D245. | 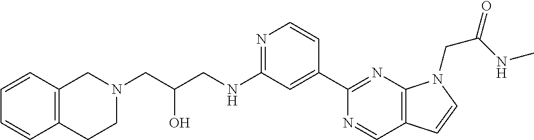 [SMILES: CNC(=O)Cn1ccc2cnc(-c3ccnc(NCC(O)CN4CCc5ccccc5C4)c3)nc21] | 471.2383 | 472.2 |
| D246. | 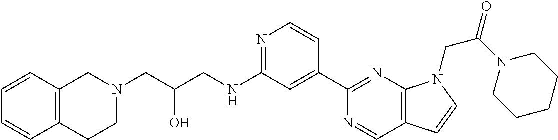 [SMILES: O=C(Cn1ccc2cnc(-c3ccnc(NCC(O)CN4CCc5ccccc5C4)c3)nc21)N1CCCCC1] | 525.2852 | 526.2 |
| D247. | 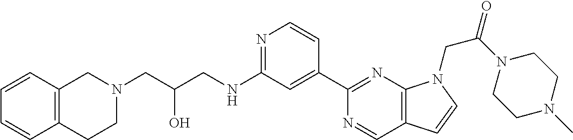 [SMILES: CN1CCN(C(=O)Cn2ccc3cnc(-c4ccnc(NCC(O)CN5CCc6ccccc6C5)c4)nc32)CC1] | 540.2961 | 541.2 |
| D248. | 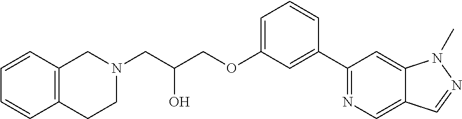 [SMILES: Cn1ncc2cnc(-c3cccc(OCC(O)CN4CCc5ccccc5C4)c3)cc21] | 414.2056 | 415.2 |
| D249. | 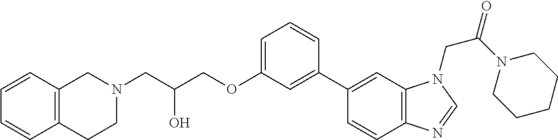 [SMILES: O=C(Cn1cnc2ccc(-c3cccc(OCC(O)CN4CCc5ccccc5C4)c3)cc21)N1CCCCC1] | 524.2787 | 525.3 |
| D250. | 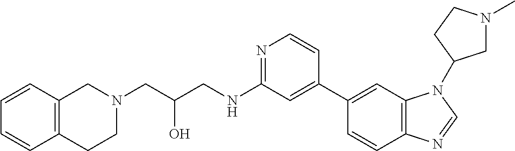 [SMILES: CN1CCC(n2cnc3ccc(-c4ccnc(NCC(O)CN5CCc6ccccc6C5)c4)cc32)C1] | 482.2794 | 483.3 |
| D251. | 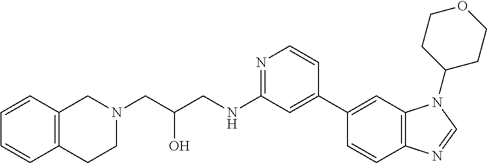 [SMILES: OC(CNc1cc(-c2ccc3ncn(C4CCOCC4)c3c2)ccn1)CN1CCc2ccccc2C1] | 483.2634 | 484.3 |
| D252. | 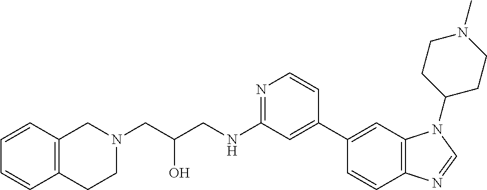 [SMILES: CN1CCC(n2cnc3ccc(-c4ccnc(NCC(O)CN5CCc6ccccc6C5)c4)cc32)CC1] | 496.2951 | 497.3 |
| D253. | 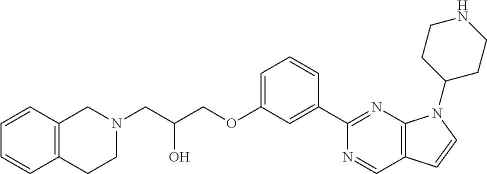 [SMILES: OC(COc1cccc(-c2ncc3ccn(C4CCNCC4)c3n2)c1)CN1CCc2ccccc2C1] | 483.2634 | 484.3 |
| D254. | 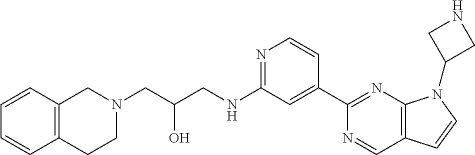 [SMILES: OC(CNc1cc(-c2ncc3ccn(C4CNC4)c3n2)ccn1)CN1CCc2ccccc2C1] | 455.2434 | 456.2 |
| D255. | 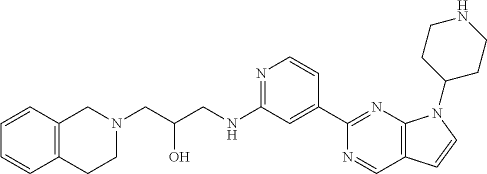 [SMILES: OC(CNc1cc(-c2ncc3ccn(C4CCNCC4)c3n2)ccn1)CN1CCc2ccccc2C1] | 483.2747 | 484.3 |
| D256. | 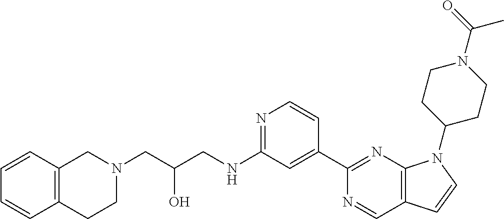 [SMILES: CC(=O)N1CCC(n2ccc3cnc(-c4ccnc(NCC(O)CN5CCc6ccccc6C5)c4)nc32)CC1] | 525.2852 | 526.3 |
| D257. | 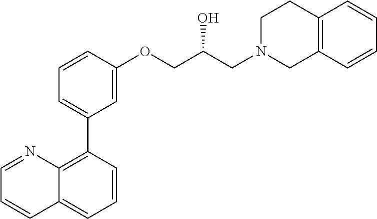 [SMILES: O[C@@H](COc1cccc(-c2cccc3cccnc23)c1)CN1CCc2ccccc2C1] | 410.1994 | 411.1 |
| D258. | 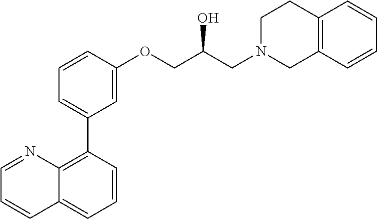 [SMILES: O[C@H](COc1cccc(-c2cccc3cccnc23)c1)CN1CCc2ccccc2C1] | 410.1994 | 411.1 |
| D259. | 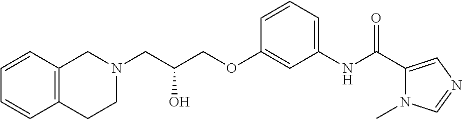 [SMILES: Cn1cncc1C(=O)Nc1cccc(OC[C@H](O)CN2CCc3ccccc3C2)c1] | 406.2005 | 407.2 |
| D260. | 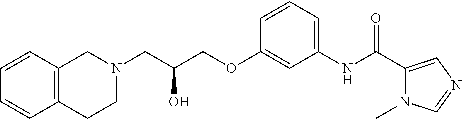 [SMILES: Cn1cncc1C(=O)Nc1cccc(OC[C@@H](O)CN2CCc3ccccc3C2)c1] | 406.2005 | 407.2 |
| D261. | 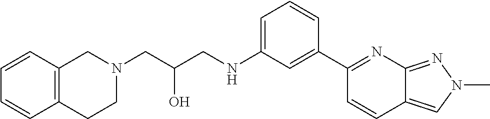 [SMILES: Cn1cc2ccc(-c3cccc(NCC(O)CN4CCc5ccccc5C4)c3)nc2n1] | 413.2216 | 414.2 |
| D262. | 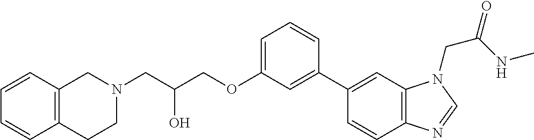 [SMILES: CNC(=O)Cn1cnc2ccc(-c3cccc(OCC(O)CN4CCc5ccccc5C4)c3)cc21] | 470.2318 | 471.3 |
| D263. | 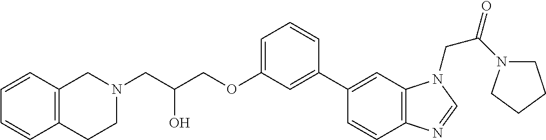 [SMILES: O=C(Cn1cnc2ccc(-c3cccc(OCC(O)CN4CCc5ccccc5C4)c3)cc21)N1CCCC1] | 510.2631 | 511.3 |
| D264. | 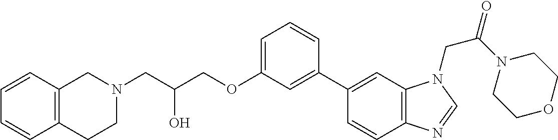 [SMILES: O=C(Cn1cnc2ccc(-c3cccc(OCC(O)CN4CCc5ccccc5C4)c3)cc21)N1CCOCC1] | 526.258 | 527.3 |
| D265. | 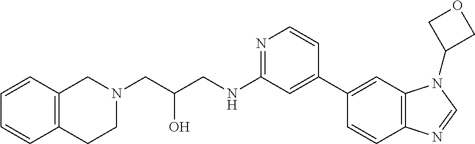 [SMILES: OC(CNc1cc(-c2ccc3ncn(C4COC4)c3c2)ccn1)CN1CCc2ccccc2C1] | 455.2321 | 456.2 |
| D266. | 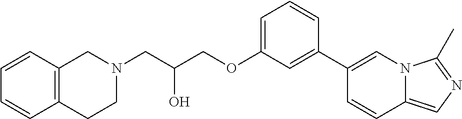 [SMILES: Cc1ncc2ccc(-c3cccc(OCC(O)CN4CCc5ccccc5C4)c3)cn12] | 413.2103 | 414.2 |
| D267. | 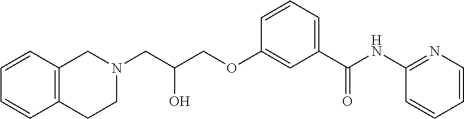 [SMILES: O=C(Nc1ccccn1)c1cccc(OCC(O)CN2CCc3ccccc3C2)c1] | 403.1896 | 404.2 |
| D268. | 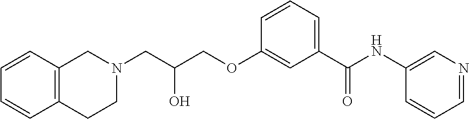 [SMILES: O=C(Nc1cccnc1)c1cccc(OCC(O)CN2CCc3ccccc3C2)c1] | 403.1896 | 404.2 |
| D269. | 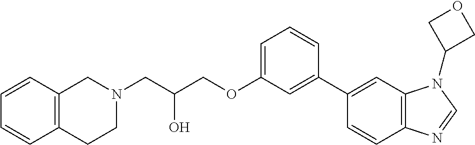 [SMILES: OC(COc1cccc(-c2ccc3ncn(C4COC4)c3c2)c1)CN1CCc2ccccc2C1] | 455.2209 | 456.2 |
| D270. | 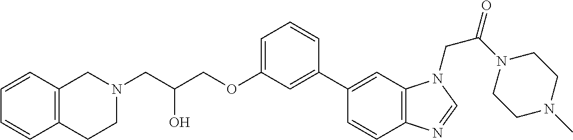 [SMILES: CN1CCN(C(=O)Cn2cnc3ccc(-c4cccc(OCC(O)CN5CCc6ccccc6C5)c4)cc32)CC1] | 539.2896 | 540.4 |
| D271. | 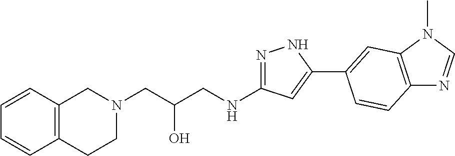 [SMILES: Cn1cnc2ccc(-c3cc(NCC(O)CN4CCc5ccccc5C4)n[nH]3)cc21] | 402.2168 | 403.2 |
| D272. | 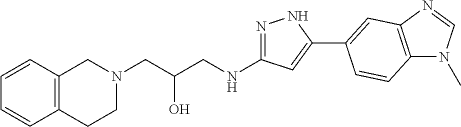 [SMILES: Cn1cnc2cc(-c3cc(NCC(O)CN4CCc5ccccc5C4)n[nH]3)ccc21] | 402.2168 | 403.2 |
| D273. | 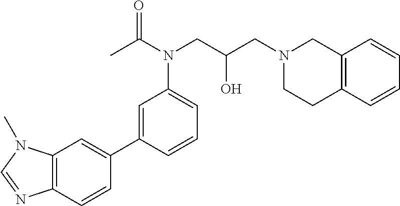 [SMILES: CC(=O)N(CC(O)CN1CCc2ccccc2C1)c1cccc(-c2ccc3ncn(C)c3c2)c1] | 454.2369 | 455.2 |
| D274. | 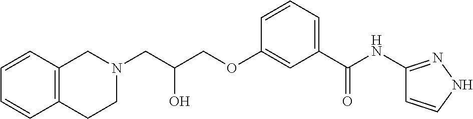 [SMILES: O=C(Nc1cc[nH]n1)c1cccc(OCC(O)CN2CCc3ccccc3C2)c1] | 392.1848 | 393.2 |
| D275. | 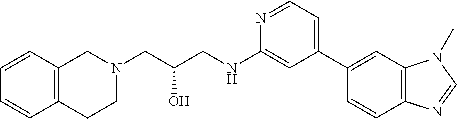 [SMILES: Cn1cnc2ccc(-c3ccnc(NC[C@H](O)CN4CCc5ccccc5C4)c3)cc21] | 413.2216 | 414.3 |
| D276. | 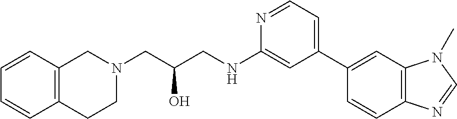 [SMILES: Cn1cnc2ccc(-c3ccnc(NC[C@@H](O)CN4CCc5ccccc5C4)c3)cc21] | 413.2216 | 414.3 |
| D277. | 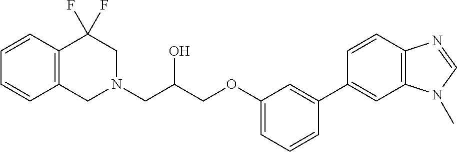 [SMILES: Cn1cnc2ccc(-c3cccc(OCC(O)CN4Cc5ccccc5C(F)(F)C4)c3)cc21] | 449.1915 | 450.1 |
| D278. | 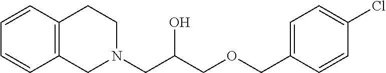 [SMILES: OC(COCc1ccc(Cl)cc1)CN1CCc2ccccc2C1] | 331.1339 | 332.1 |
| D279. | 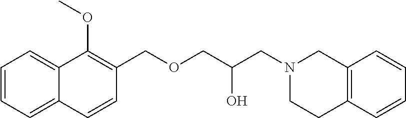 [SMILES: COc1c(COCC(O)CN2CCc3ccccc3C2)ccc2ccccc12] | 377.1991 | 378.0 |
| D280. | 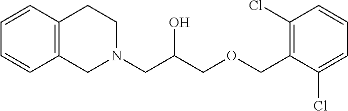 [SMILES: OC(COCc1c(Cl)cccc1Cl)CN1CCc2ccccc2C1] | 365.0949 | 366.0 |
| D281. | 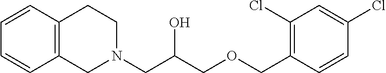 [SMILES: OC(COCc1ccc(Cl)cc1Cl)CN1CCc2ccccc2C1] | 365.0949 | 366.0 |
| D282. | 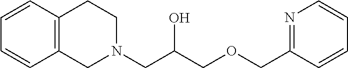 [SMILES: OC(COCc1ccccn1)CN1CCc2ccccc2C1] | 298.1681 | 299.1 |
| D283. | 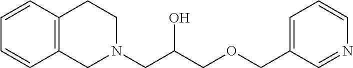 [SMILES: OC(COCc1cccnc1)CN1CCc2ccccc2C1] | 298.1681 | 299.1 |
| D284. | 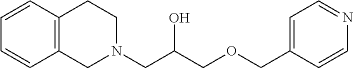 [SMILES: OC(COCc1ccncc1)CN1CCc2ccccc2C1] | 298.1681 | 299.1 |
| D285. | 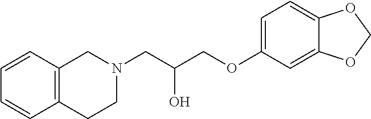 [SMILES: OC(COc1ccc2c(c1)OCO2)CN1CCc2ccccc2C1] | 327.1471 | 327.9 |
| D286. | 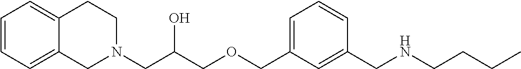 [SMILES: CCCCNCc1cccc(COCC(O)CN2CCc3ccccc3C2)c1] | 382.262 | 383.2 |
| D287. | 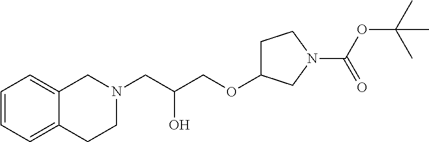 [SMILES: CC(C)(C)OC(=O)N1CCC(OCC(O)CN2CCc3ccccc3C2)C1] | 376.2362 | 377.2 |
| D288. | 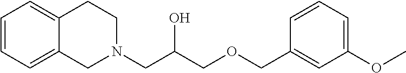 [SMILES: COc1cccc(COCC(O)CN2CCc3ccccc3C2)c1] | 327.1834 | 328.0 |
| D289. | 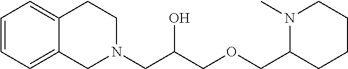 [SMILES: CN1CCCCC1COCC(O)CN1CCc2ccccc2C1] | 318.2307 | 319.1 |
| D290. | 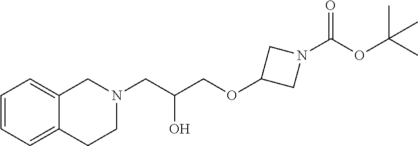 [SMILES: CC(C)(C)OC(=O)N1CC(OCC(O)CN2CCc3ccccc3C2)C1] | 362.2206 | 363.1 |
| D291. | 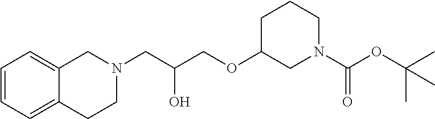 [SMILES: CC(C)(C)OC(=O)N1CCCC(OCC(O)CN2CCc3ccccc3C2)C1] | 390.2519 | 390.9 |
| D292. | 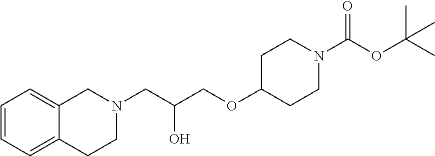 [SMILES: CC(C)(C)OC(=O)N1CCC(OCC(O)CN2CCc3ccccc3C2)CC1] | 390.2519 | 391.2 |
| D293. | 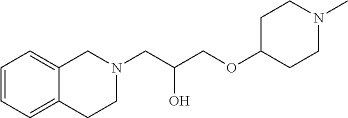 [SMILES: CN1CCC(OCC(O)CN2CCc3ccccc3C2)CC1] | 304.2151 | 305.1 |
| D294. | 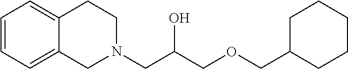 [SMILES: OC(COCC1CCCCC1)CN1CCc2ccccc2C1] | 303.2198 | 304.2 |
| D295. | 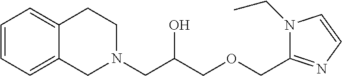 [SMILES: CCn1ccnc1COCC(O)CN1CCc2ccccc2C1] | 315.1947 | 316.0 |
| D296. | 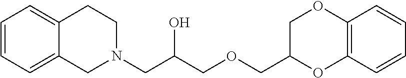 [SMILES: OC(COCC1COc2ccccc2O1)CN1CCc2ccccc2C1] | 277.1678 | 278.1 |
| D297. | 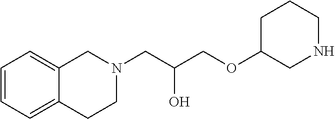 [SMILES: OC(COC1CCCNC1)CN1CCc2ccccc2C1] | 290.1994 | 291.1 |
| D298. | 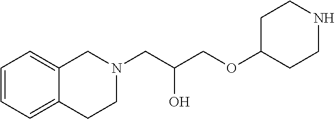 [SMILES: OC(COC1CCNCC1)CN1CCc2ccccc2C1] | 290.1994 | 291.1 |
| D299. | 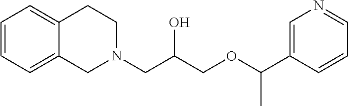 [SMILES: CC(OCC(O)CN1CCc2ccccc2C1)c1cccnc1] | 312.1838 | 313.0 |
| D300. | 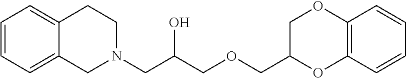 [SMILES: OC(COCC1COc2ccccc2O1)CN1CCc2ccccc2C1] | 355.1784 | 356.1 |
| D301. | 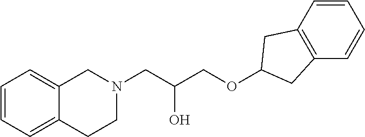 [SMILES: OC(COC1Cc2ccccc2C1)CN1CCc2ccccc2C1] | 323.1885 | 324.0 |
| D302. | 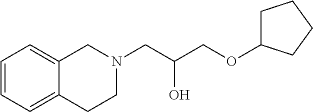 [SMILES: OC(COC1CCCC1)CN1CCc2ccccc2C1] | 275.1885 | 276.1 |
| D303. | 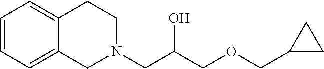 [SMILES: OC(COCC1CC1)CN1CCc2ccccc2C1] | 261.1729 | 262.1 |
| D304. | 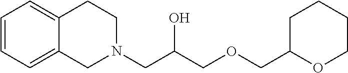 [SMILES: OC(COCC1CCCCO1)CN1CCc2ccccc2C1] | 305.1991 | 306.1 |
| D305. | 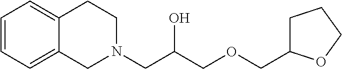 [SMILES: OC(COCC1CCCO1)CN1CCc2ccccc2C1] | 291.1834 | 292.1 |
| D306. | 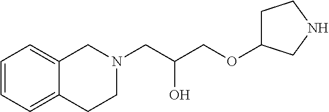 [SMILES: OC(COC1CCNC1)CN1CCc2ccccc2C1] | 276.1838 | 277.1 |
| D307. | 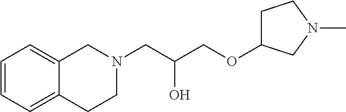 [SMILES: CN1CCC(OCC(O)CN2CCc3ccccc3C2)C1] | 290.1994 | 291.1 |
| D308. | 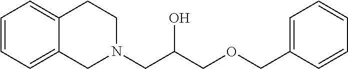 [SMILES: OC(COCc1ccccc1)CN1CCc2ccccc2C1] | 297.1729 | 297.9 |
| D309. | 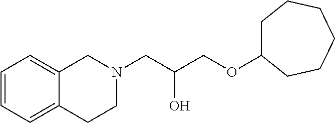 [SMILES: OC(COC1CCCCCC1)CN1CCc2ccccc2C1] | 303.2198 | 304.1 |
| D310. | 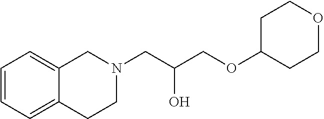 [SMILES: OC(COC1CCOCC1)CN1CCc2ccccc2C1] | 291.1834 | 292.1 |

In certain embodiments, a provided compound is a compound listed in Table E-1, or a pharmaceutically acceptable salt thereof

TABLE E-1
Exemplary Compounds

| Cmpd | | | LC-MS m/z |
|---|---|---|---|
| No | Structure | Exact mass | (M + H) |
| E1. | 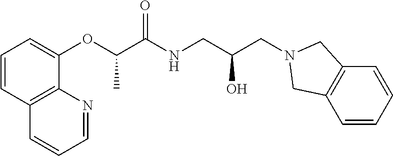 [SMILES: C[C@H](Oc1cccc2cccnc12)C(=O)NC[C@H](O)CN1Cc2ccccc2C1] | 391.1896 | 392.1 |
| E2. | 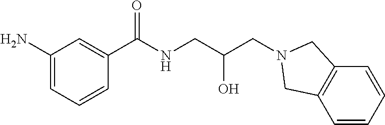 [SMILES: Nc1cccc(C(=O)NCC(O)CN2Cc3ccccc3C2)c1] | 311.1634 | 312.2 |
| E3. | 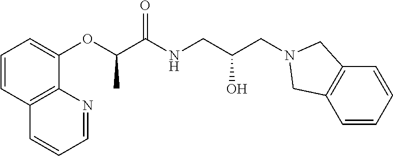 [SMILES: C[C@@H](Oc1cccc2cccnc12)C(=O)NC[C@@H](O)CN1Cc2ccccc2C1] | 391.1896 | 392.1 |
| E4. | 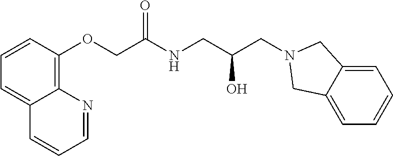 [SMILES: O=C(COc1cccc2cccnc12)NC[C@H](O)CN1Cc2ccccc2C1] | 377.1739 | 378.1 |
| E5. | 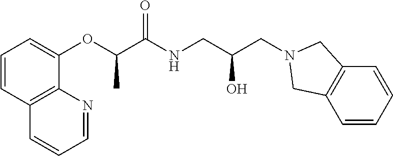 [SMILES: C[C@@H](Oc1cccc2cccnc12)C(=O)NC[C@H](O)CN1Cc2ccccc2C1] | 391.1896 | 392.1 |
| E6. | 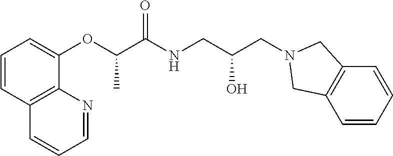 [SMILES: C[C@H](Oc1cccc2cccnc12)C(=O)NC[C@@H](O)CN1Cc2ccccc2C1] | 391.1896 | 392.1 |
| E7. | 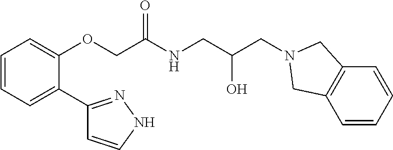 [SMILES: O=C(COc1ccccc1-c1cc[nH]n1)NCC(O)CN1Cc2ccccc2C1] | 392.1848 | 393.2 |
| E8. | 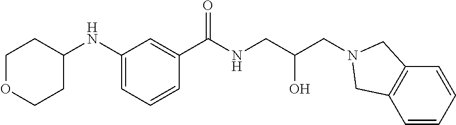 [SMILES: O=C(NCC(O)CN1Cc2ccccc2C1)c1cccc(NC2CCOCC2)c1] | 395.2209 | 396.2 |
| E9. | 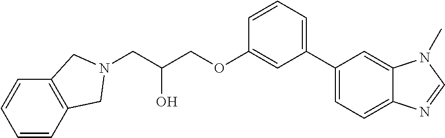 [SMILES: Cn1cnc2ccc(-c3cccc(OCC(O)CN4Cc5ccccc5C4)c3)cc21] | 399.1947 | 400.2 |
| E10. | 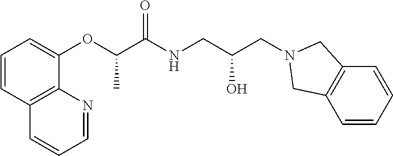 [SMILES: C[C@H](Oc1cccc2cccnc12)C(=O)NC[C@@H](O)CN1Cc2ccccc2C1] | 397.2002 | 398.2 |
| E11. | 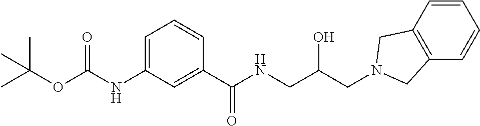 [SMILES: CC(C)(C)OC(=O)Nc1cccc(C(=O)NCC(O)CN2Cc3ccccc3C2)c1] | 411.2158 | 412.2 |
| E12. | 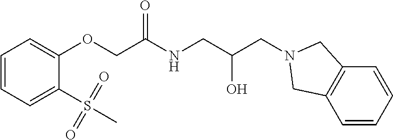 [SMILES: CS(=O)(=O)c1ccccc1OCC(=O)NCC(O)CN1Cc2ccccc2C1] | 404.1406 | 405.2 |
| E13. | 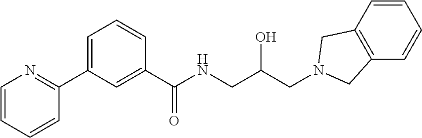 [SMILES: O=C(NCC(O)CN1Cc2ccccc2C1)c1cccc(-c2ccccn2)c1] | 373.179 | 374.2 |
| E14. | 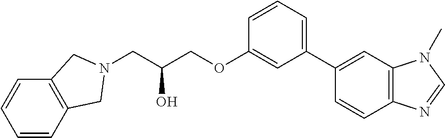 [SMILES: Cn1cnc2ccc(-c3cccc(OC[C@@H](O)CN4Cc5ccccc5C4)c3)cc21] | 399.1947 | 400.2 |
| E15. | 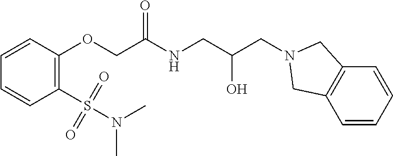 [SMILES: CN(C)S(=O)(=O)c1ccccc1OCC(=O)NCC(O)CN1Cc2ccccc2C1] | 433.1671 | 434.1 |
| E16. | 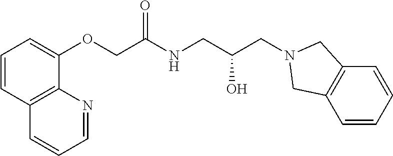 [SMILES: O=C(COc1cccc2cccnc12)NC[C@@H](O)CN1Cc2ccccc2C1] | 377.1739 | 378.1 |
| E17. | 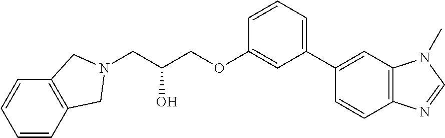 [SMILES: Cn1cnc2ccc(-c3cccc(OC[C@H](O)CN4Cc5ccccc5C4)c3)cc21] | 399.1947 | 400.2 |
| E18. | 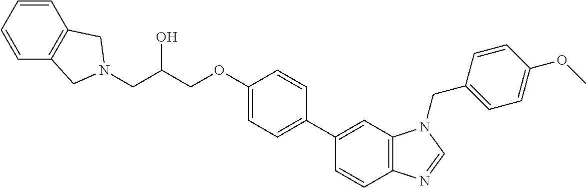 [SMILES: COc1ccc(Cn2cnc3ccc(-c4ccc(OCC(O)CN5Cc6ccccc6C5)cc4)cc32)cc1] | 505.2365 | 506.2 |
| E19. | 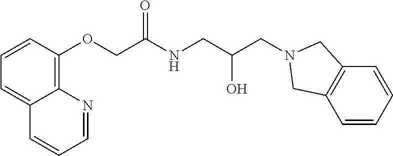 [SMILES: O=C(COc1cccc2cccnc12)NCC(O)CN1Cc2ccccc2C1] | 377.1739 | 378.1 |
| E20. | 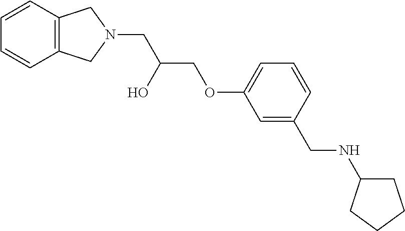 [SMILES: OC(COc1cccc(CNC2CCCC2)c1)CN1Cc2ccccc2C1] | 366.2307 | 367.2 |

In certain embodiments, a provided compound inhibits PRMT5. In certain embodiments, a provided compound inhibits wild-type PRMT5. In certain embodiments, a provided compound inhibits a mutant PRMT5. In certain embodiments, a provided compound inhibits PRMT5, e.g., as measured in an assay described herein. In certain embodiments, the PRMT5 is from a human. In certain embodiments, a provided compound inhibits PRMT5 at an IC₅₀ less than or equal to 10 In certain embodiments, a provided compound inhibits PRMT5 at an IC₅₀ less than or equal to 1 In certain embodiments, a provided compound inhibits PRMT5 at an IC₅₀ less than or equal to 0.1 In certain embodiments, a provided compound inhibits PRMT5 in a cell at an EC₅₀ less than or equal to 10 μM. In certain embodiments, a provided compound inhibits PRMT5 in a cell at an EC₅₀ less than or equal to 1 In certain embodiments, a provided compound inhibits PRMT5 in a cell at an EC₅₀ less than or equal to 0.1 In certain embodiments, a provided compound inhibits cell proliferation at an EC₅₀ less than or equal to 10 In certain embodiments, a provided compound inhibits cell proliferation at an EC₅₀ less than or equal to 1 In certain embodiments, a provided compound inhibits cell proliferation at an EC₅₀ less than or equal to 0.1 In some embodiments, a provided compound is selective for PRMT5 over other methyltransferases. In certain embodiments, a provided compound is at least about 10-fold selective, at least about 20-fold selective, at least about 30-fold selective, at least about 40-fold selective, at least about 50-fold selective, at least about 60-fold selective, at least about 70-fold selective, at least about 80-fold selective, at least about 90-fold selective, or at least about 100-fold selective for PRMT5 relative to one or more other methyltransferases.

It will be understood by one of ordinary skill in the art that the PRMT5 can be wild-type PRMT5, or any mutant or variant of PRMT5.

In certain embodiments, the PRMT5 is isoform A (GenBank accession no. NP006100) (SEQ ID NO.:1):

MAAMAVGGAG GSRVSSGRDL NCVPEIADTL GAVAKQGFDF
LCMPVFHPRF KREFIQEPAK NRPGPQTRSD LLLSGRDWNT
LIVGKLSPWI RPDSKVEKIR RNSEAAMLQE LNFGAYLGLP
AFLLPLNQED NTNLARVLTN HIHTGHHSSM FWMRVPLVAP
EDLRDDIIEN APTTHTEEYS GEEKTWMWWH NFRTLCDYSK
RIAVALEIGA DLPSNHVIDR WLGEPIKAAI LPTSIFLTNK
KGFPVLSKMH QRLIFRLLKL EVQFIITGTN HHSEKEFCSY
LQYLEYLSQN RPPPNAYELF AKGYEDYLQS PLQPLMDNLE
SQTYEVFEKD PIKYSQYQQA IYKCLLDRVP EEEKDTNVQV
LMVLGAGRGP LVNASLRAAK QADRRIKLYA VEKNPNAVVT
LENWQFEEWG SQVTVVSSDM REWVAPEKAD IIVSELLGSF
ADNELSPECL DGAQHFLKDD GVSIPGEYTS FLAPISSSKL
YNEVRACREK DRDPEAQFEM PYVVRLHNFH QLSAPQPCFT
FSHPNRDPMI DNNRYCTLEF PVEVNTVLHG FAGYFETVLY
QDITLSIRPE THSPGMFSWF PILFPIKQPI TVREGQTICV
RFWRCSNSKK VWYEWAVTAP VCSAIHNPTG RSYTIGL

In certain embodiments, the PRMT5 is isoform B (GenBank accession no. NP001034708) (SEQ ID NO.:2)

MRGPNSGTEK GRLVIPEKQG FDFLCMPVFH PRFKREFIQE
PAKNRPGPQT RSDLLLSGRD WNTLIVGKLS PWIRPDSKVE
KIRRNSEAAM LQELNFGAYL GLPAFLLPLN QEDNTNLARV
LTNHIHTGHH SSMFWMRVPL VAPEDLRDDI IENAPTTHTE
EYSGEEKTWM WWHNFRTLCD YSKRIAVALE IGADLPSNHV
IDRWLGEPIK AAILPTSIFL TNKKGFPVLS KMHQRLIFRL
LKLEVQFIIT GTNHHSEKEF CSYLQYLEYL SQNRPPPNAY
ELFAKGYEDY LQSPLQPLMD NLESQTYEVF EKDPIKYSQY
QQAIYKCLLD RVPEEEKDTN VQVLMVLGAG RGPLVNASLR
AAKQADRRIK LYAVEKNPNA VVTLENWQFE EWGSQVTVVS
SDMREWVAPE KADIIVSELL GSFADNELSP ECLDGAQHFL
KDDGVSIPGE YTSFLAPISS SKLYNEVRAC REKDRDPEAQ
FEMPYVVRLH NFHQLSAPQP CFTFSHPNRD PMIDNNRYCT
LEFPVEVNTV LHGFAGYFET VLYQDITLSI RPETHSPGMF
SWFPILFPIK QPITVREGQT ICVRFWRCSN SKKVWYEWAV
TAPVCSAIHN PTGRSYTIGL

In certain embodiments, the PRMT5 is transcript variant 1 (GenBank accession no. NM_{—}006109).

### PHARMACEUTICAL COMPOSITIONS AND ADMINISTRATION

The present disclosure provides pharmaceutical compositions comprising a compound described herein, e.g., a compound of Formula (I), or a pharmaceutically acceptable salt thereof, as described herein, and optionally a pharmaceutically acceptable excipient. It will be understood by one of ordinary skill in the art that the compounds described herein, or salts thereof, may be present as hydrates, solvates, or polymorphs. In certain embodiments, a provided composition comprises two or more compounds described herein. In certain embodiments, a compound described herein, or a pharmaceutically acceptable salt thereof, is provided in an effective amount in the pharmaceutical composition. In certain embodiments, the effective amount is a therapeutically effective amount. In certain embodiments, the effective amount is an amount effective for inhibiting PRMT5. In certain embodiments, the effective amount is an amount effective for treating a PRMT5-mediated disorder. In certain embodiments, the effective amount is a prophylactically effective amount. In certain embodiments, the effective amount is an amount effective to prevent a PRMT5-mediated disorder.

Pharmaceutically acceptable excipients include any and all solvents, diluents, or other liquid vehicles, dispersions, suspension aids, surface active agents, isotonic agents, thickening or emulsifying agents, preservatives, solid binders, lubricants, and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing a compound described herein (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the present disclosure will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60], sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the compounds described herein are mixed with solubilizing agents such as Cremophor™, alcohols, oils, modified oils, glycols, polysorbates, cyclodextrins, polymers, and mixtures thereof.

Injectable preparations, for example, sterile injecTable Aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a provided compound may include ointments, pastes, creams, lotions, gels, powders, solutions, sprays, inhalants and/or patches. Generally, the active ingredient is admixed under sterile conditions with a pharmaceutically acceptable carrier and/or any desired preservatives and/or buffers as can be required. Additionally, the present disclosure encompasses the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions can be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes can be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A provided pharmaceutical composition can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A provided pharmaceutical composition can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A provided pharmaceutical composition can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, a compound described herein is administered one or more times per day, for multiple days. In some embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In certain embodiments, the additional therapeutically active agent is a compound of Formula (I). In certain embodiments, the additional therapeutically active agent is not a compound of Formula (I). In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of a provided compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the present discosure are kits (e.g., pharmaceutical packs). The kits provided may comprise a provided pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of a provided pharmaceutical composition or compound. In some embodiments, a provided pharmaceutical composition or compound provided in the container and the second container are combined to form one unit dosage form. In some embodiments, a provided kits further includes instructions for use.

### Methods of Use and Treatment

Compounds and compositions described herein are generally useful for the inhibition of PRMT5. In some embodiments, methods of treating PRMT5-mediated disorder in a subject are provided which comprise administering an effective amount of a compound described herein (e.g., a compound of Formula (I)), or a pharmaceutically acceptable salt thereof), to a subject in need of treatment. In certain embodiments, the effective amount is a therapeutically effective amount. In certain embodiments, the effective amount is a prophylactically effective amount. In certain embodiments, the subject is suffering from a PRMT5-mediated disorder. In certain embodiments, the subject is susceptible to a PRMT5-mediated disorder.

As used herein, the term “PRMT5-mediated disorder” means any disease, disorder, or other pathological condition in which PRMT5 is known to play a role. Accordingly, in some embodiments, the present disclosure relates to treating or lessening the severity of one or more diseases in which PRMT5 is known to play a role.

In some embodiments, the present disclosure provides a method of inhibiting PRMT5 comprising contacting PRMT5 with an effective amount of a compound described herein (e.g., a compound of Formula (I)), or a pharmaceutically acceptable salt thereof. The PRMT5 may be purified or crude, and may be present in a cell, tissue, or subject. Thus, such methods encompass both inhibition of in vitro and in vivo PRMT5 activity. In certain embodiments, the method is an in vitro method, e.g., such as an assay method. It will be understood by one of ordinary skill in the art that inhibition of PRMT5 does not necessarily require that all of the PRMT5 be occupied by an inhibitor at once. Exemplary levels of inhibition of PRMT5 include at least 10% inhibition, about 10% to about 25% inhibition, about 25% to about 50% inhibition, about 50% to about 75% inhibition, at least 50% inhibition, at least 75% inhibition, about 80% inhibition, about 90% inhibition, and greater than 90% inhibition.

In some embodiments, provided is a method of inhibiting PRMT5 activity in a subject in need thereof comprising administering to the subject an effective amount of a compound described herein (e.g., a compound of Formula (I)), or a pharmaceutically acceptable salt thereof.

In certain embodiments, provided is a method of altering gene expression in a cell which comprises contacting a cell with an effective amount of a compound of Formula (I), or a pharmaceutically acceptable salt thereof. In certain embodiments, the cell in culture in vitro. In certain embodiments, the cell is in an animal, e.g., a human. In certain embodiments, the cell is in a subject in need of treatment.

In certain embodiments, provided is a method of altering transcription in a cell which comprises contacting a cell with an effective amount of a compound of Formula (I), or a pharmaceutically acceptable salt thereof. In certain embodiments, the cell in culture in vitro. In certain embodiments, the cell is in an animal, e.g., a human. In certain embodiments, the cell is in a subject in need of treatment.

In some embodiments, a provided compound is useful in treating a proliferative disorder, such as cancer, a benign neoplasm, an autoimmune disease, or an inflammatory disease. For example, while not being bound to any particular mechanism, PRMT5 has been shown to be involved in cyclin D1 dysregulated cancers. Increased PRMT5 activity mediates key events associated with cyclin D1-dependent neoplastic growth including CUL4 repression, CDT1 overexpression, and DNA re-replication. Further, human cancers harboring mutations in Fbx4, the cyclin D1 E3 ligase, exhibit nuclear cyclin D1 accumulation and increased PRMT5 activity (Aggarwal et al., Cancer Cell. 2010 18(4):329-40). Additionally, PRMT5 has also been implicated in accelerating cell cycle progression through G1 phase and modulating regulators of G1; for example, PRMT5 may upregulate cyclin-dependent kinase (CDK) 4, CDK6, and cyclins D1, D2 and El. Moreover, PRMT5 may activate phosphoinositide 3-kinase (PI3K)/AKT signaling (Wei et al., Cancer Sci. 2012 103(9):1640-50). Thus in some embodiments, the inhibition of PRMT5 by a provided compound is useful in treating the following non-limiting list of cancers: breast cancer, esophageal cancer, bladder cancer, lung cancer, hematopoietic cancer, lymphoma, medulloblastoma, rectum adenocarcinoma, colon adenocarcinoma, gastric cancer, pancreatic cancer, liver cancer, adenoid cystic carcinoma, lung adenocarcinoma, head and neck squamous cell carcinoma, brain tumors, hepatocellular carcinoma, renal cell carcinoma, melanoma, oligodendroglioma, ovarian clear cell carcinoma, and ovarian serous cystadenocarcinoma.

In some embodiments, the inhibition of PRMT5 by a provided compound is useful in treating prostate cancer and lung cancer, in which PRMT5 has been shown to play a role (Gu et al., PLoS One 2012; 7(8):e44033; Gu et al., Biochem. J. (2012) 446 (235-241)). In some embodiments, a provided compound is useful to delay the onset of, slow the progression of, or ameliorate the symptoms of cancer. In some embodiments, a provided compound is administered in combination with other compounds, drugs, or therapeutics to treat cancer.

In some embodiments, compounds described herein are useful for treating a cancer including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, a provided compound is useful in treating a metabolic disorder, such as diabetes or obesity. For example, while not being bound to any particular mechanism, a role for PRMT5 has been recognized in adipogenesis Inhibition of PRMT5 expression in multiple cell culture models for adipogenesis prevented the activation of adipogenic genes, while overexpression of PRMT5 enhanced adipogenic gene expression and differentiation (LeBlanc et al., Mol Endocrinol. 2012 April; 26(4):583-97). Additionally, it has been shown that adipogenesis plays a pivotal role in the etiology and progression of diabetes and obesity (Camp et al., Trends Mol Med. 2002 September; 8(9):442-7). Thus in some embodiments, the inhibition of PRMT5 by a provided compound is useful in treating diabetes and/or obesity.

In some embodiments, a provided compound is useful to delay the onset of, slow the progression of, or ameliorate the symptoms of, diabetes. In some embodiments, the diabetes is Type 1 diabetes. In some embodiments, the diabetes is Type 2 diabetes. In some embodiments, a provided compound is useful to delay the onset of, slow the progression of, or ameliorate the symptoms of, obesity. In some embodiments, a provided compound could be used in combination with other compounds, drugs, or therapeutics, such as metformin and insulin, to treat diabetes and/or obesity.

In some embodiments, a provided compound is useful in treating a blood disorder, such as sickle cell disease or 3-thalassemia. For example, while not being bound to any particular mechanism, PRMT5 is a known repressor of γ-globin gene expression, and increased fetal γ-globin (HbF) levels in adulthood are associated with symptomatic amelioration in sickle cell disease and 3-thalassemia (Xu et al., Haematologica. 2012 November; 97(11):1632-40). Thus in some embodiments, the inhibition of PRMT5 by a provided compound is useful in treating a blood disorder, such as sickle cell disease or β-thalassemia.

In some embodiments, a provided compound is useful to delay the onset of, slow the progression of, or ameliorate the symptoms of, sickle cell disease. In some embodiments, a provided compound is useful to delay the onset of, slow the progression of, or ameliorate the symptoms of, β-thalassemia. In some embodiments, a provided compound could be used in combination with other compounds, drugs, or therapeutics, to treat sickle cell disease or β-thalassemia.

### Synthetic Methods

In some embodiments, compounds described herein can prepared using methods shown in general Scheme A-1. Compound Bb can be prepared via ring opening of a chiral or racemic epoxide group. This amino alcohol intermediate can be coupled to form an amide via normal amide coupling methodology using a carboxylic acid Aa wherein Z is hydrogen or via amination of an ester of intermediate Aa when Z is an optionally substituted aliphatic group.

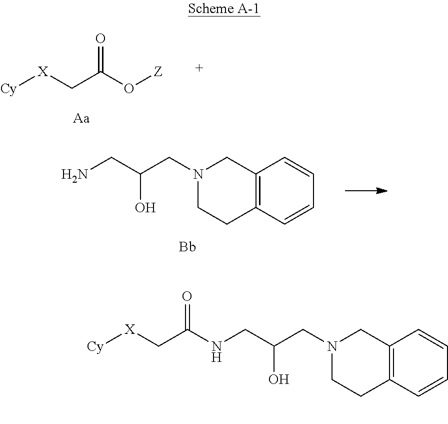

For example, exemplary Schemes A-2 and A-3 show such couplings.

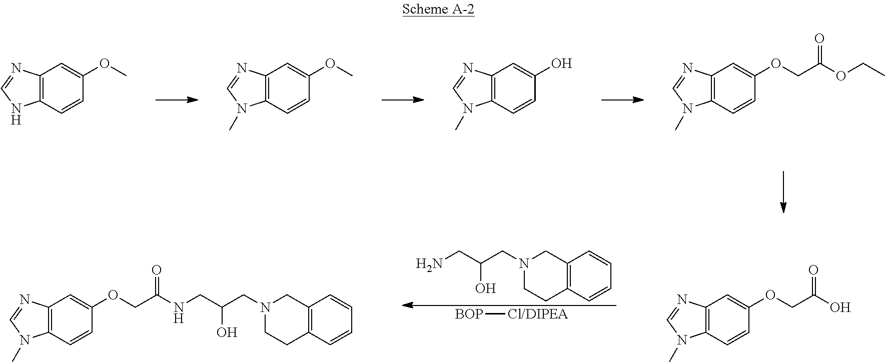

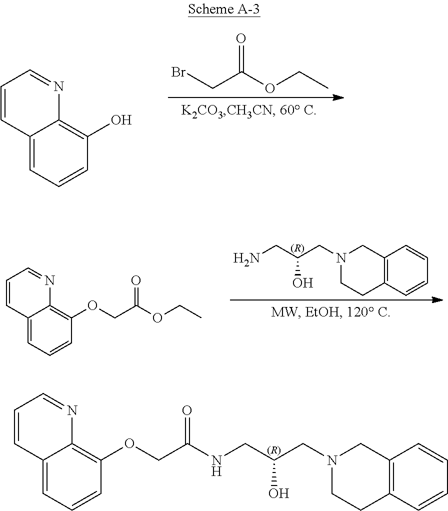

In some embodiments, an amide coupling step can be used to provide a key intermediate for further synthesis, as shown, for example, in exemplary Scheme A-4.

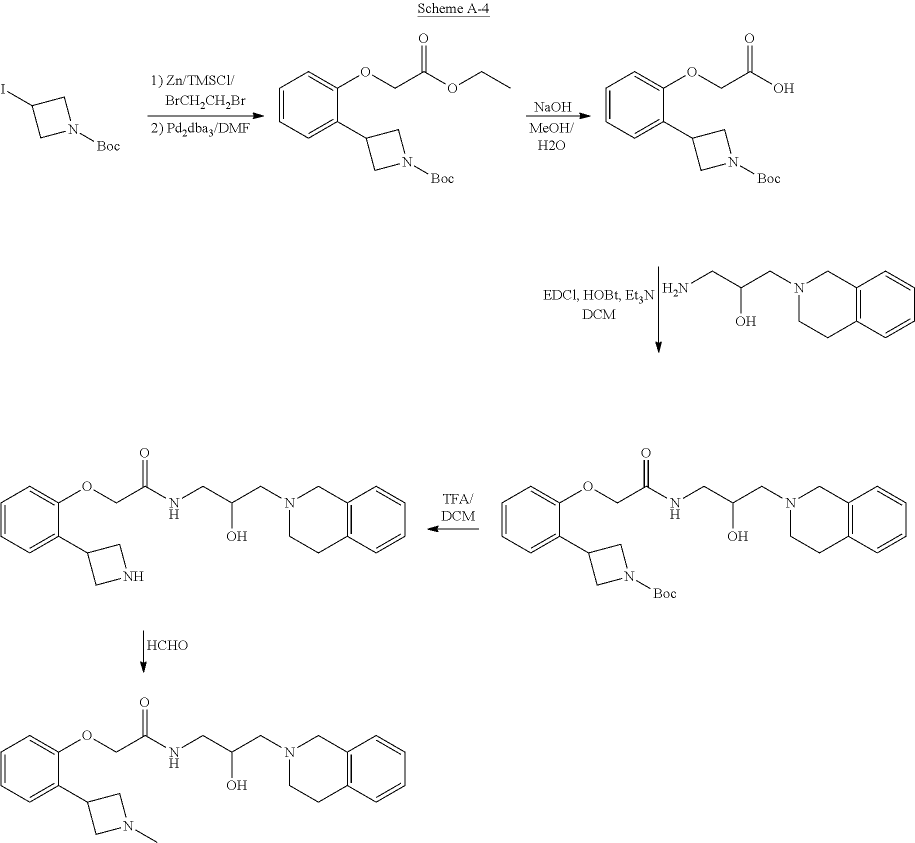

In some embodiments, compounds described herein can prepared using methods shown in general Scheme B-1. Compound Bb can be prepared via ring opening of a chiral or racemic epoxide group. This amino alcohol intermediate can be coupled to form an amide via normal amide coupling methodology using a carboxylic acid Cc wherein Z is hydrogen or via amination of an ester of intermediate Cc when Z is an optionally substituted aliphatic group.

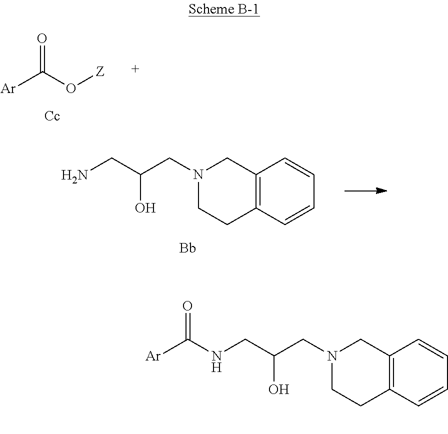

Analogous reactions may be performed to form a carbamate or urea bond using methods known to one of ordinary skill in the art.

In some embodiments, such couplings can be used to provide a key intermediate for further synthesis, as shown, for example, in exemplary Scheme B-2.

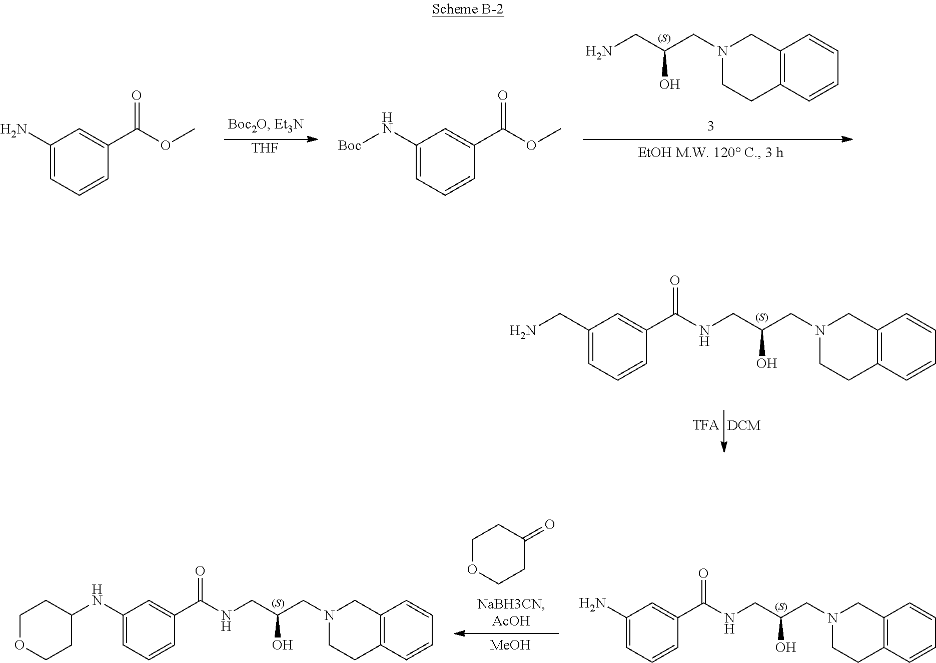

In other embodiments, an amide coupling step is the final synthetic step as shown in exemplary Scheme B-3.

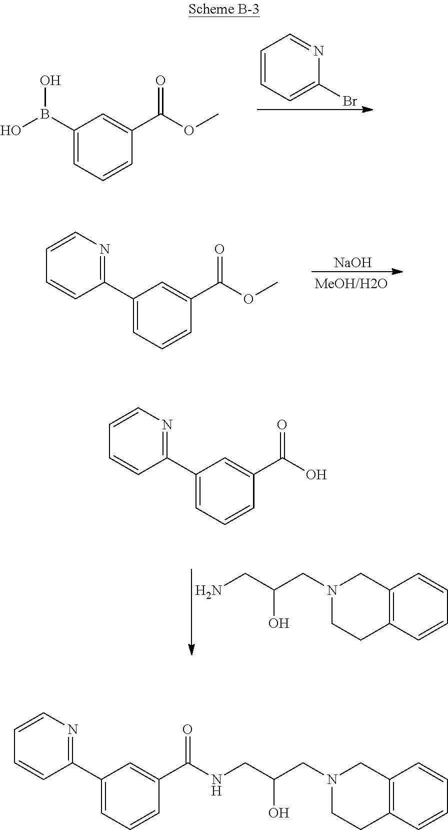

In some embodiments, compounds described herein can prepared using methods shown in general Scheme C-1, which describes ring opening of a chiral or racemic epoxide group to form the amino alcohol moiety linker.

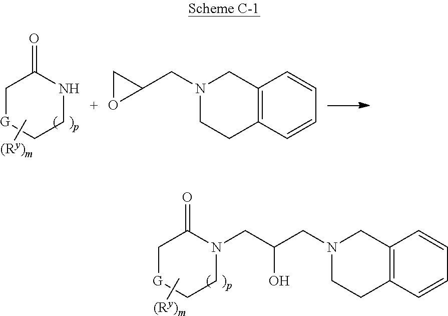

In some embodiments, epoxide opening can be performed in the final step as shown in exemplary Schemes C-2 and C-3.

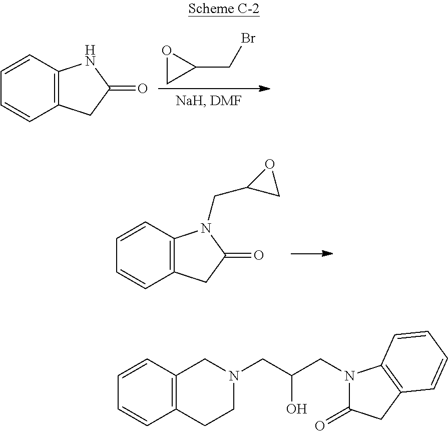

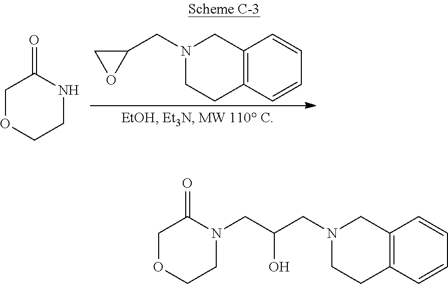 [SMILES: O=C1COCCN1 ; c1ccc2c(c1)CCN(CC1CO1)C2 ; O=C1COCCN1CC(O)CN1CCc2ccccc2C1]

In some embodiments, an amide coupling step can be used to provide a key intermediate for further synthesis, as shown in exemplary Schemes C-4 to C-6.

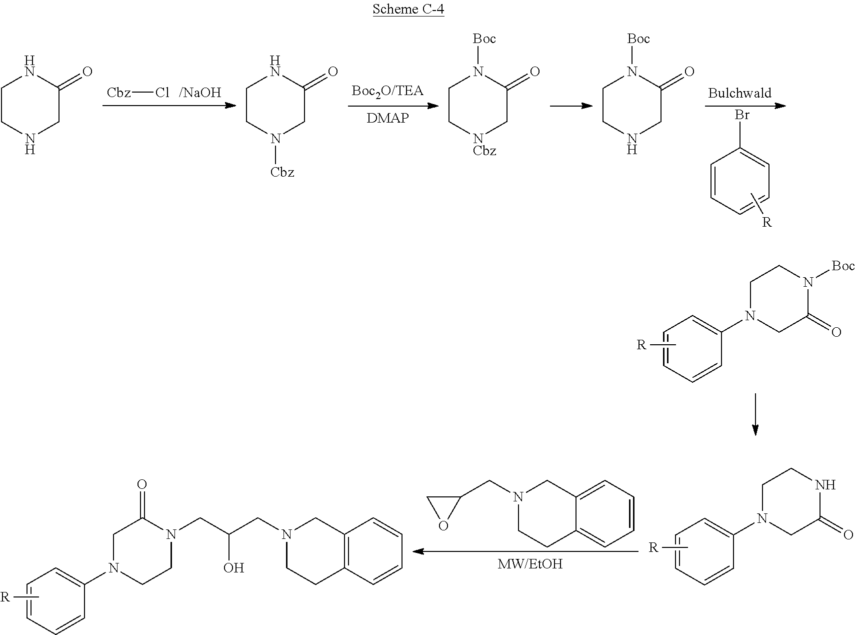

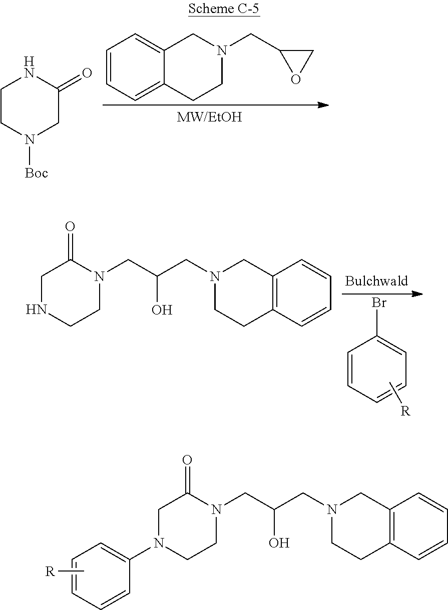

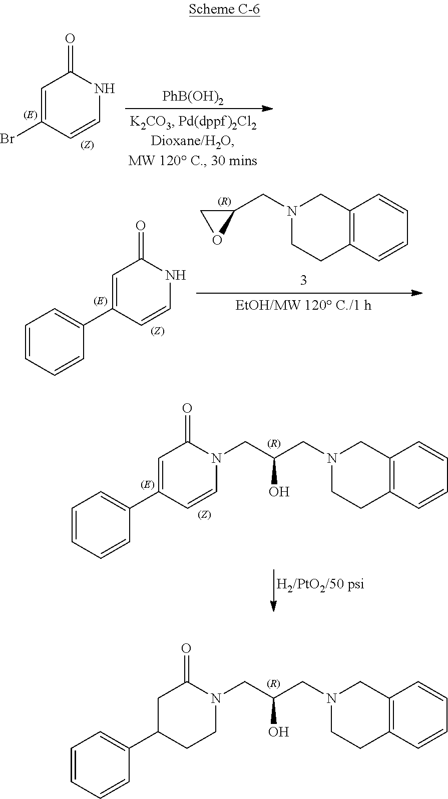

In some embodiments, compounds described herein can prepared using methods shown in general Scheme D-1, comprising a ring opening of a chiral or racemic epoxide group.

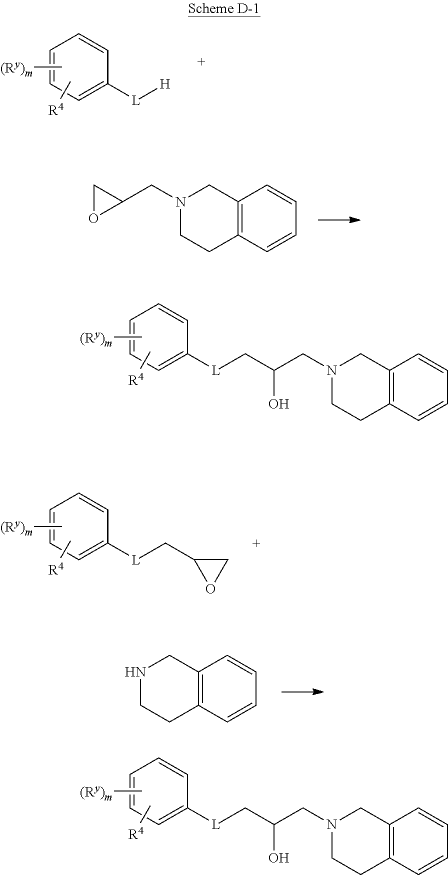

In some embodiments, the epoxide opening is the final step in the synthesis, as shown in exemplary Scheme D-2.

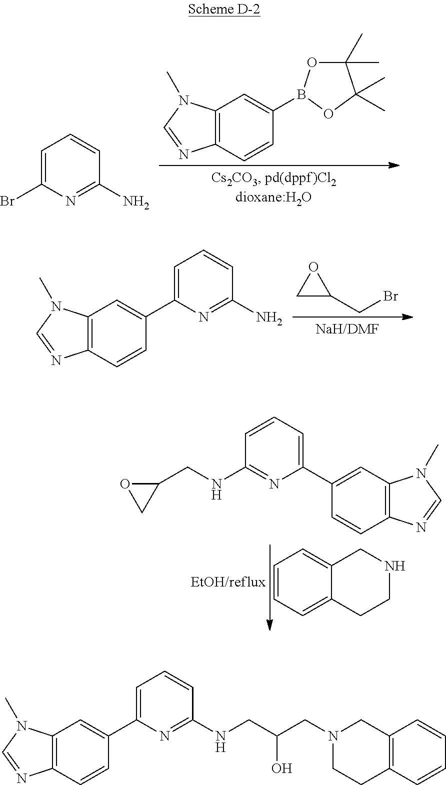

In some embodiments, epoxide opening is employed to build key intermediates for addition synthesis as shown in exemplary schemes D-3 to D-6.

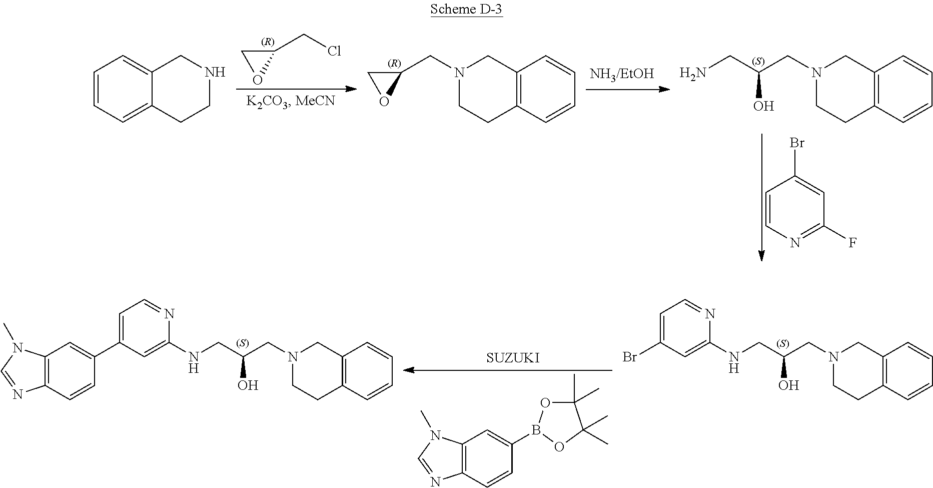

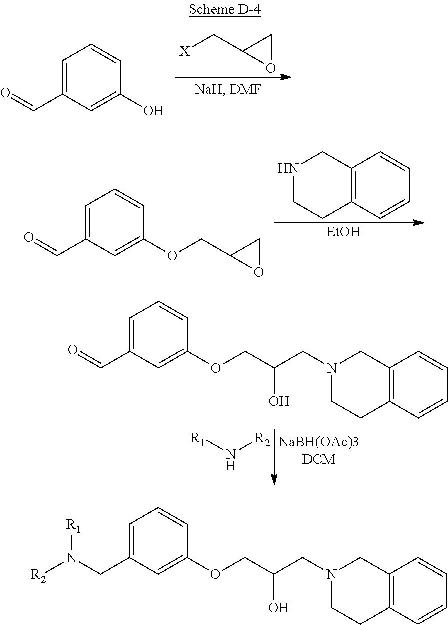

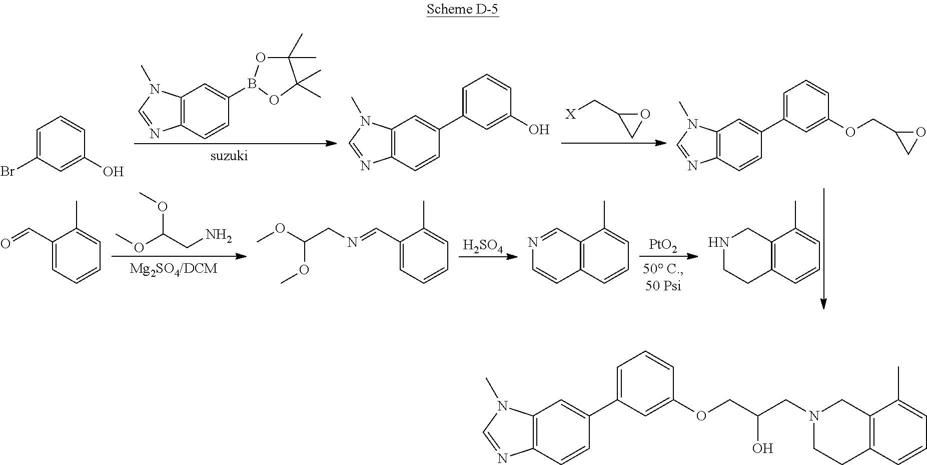

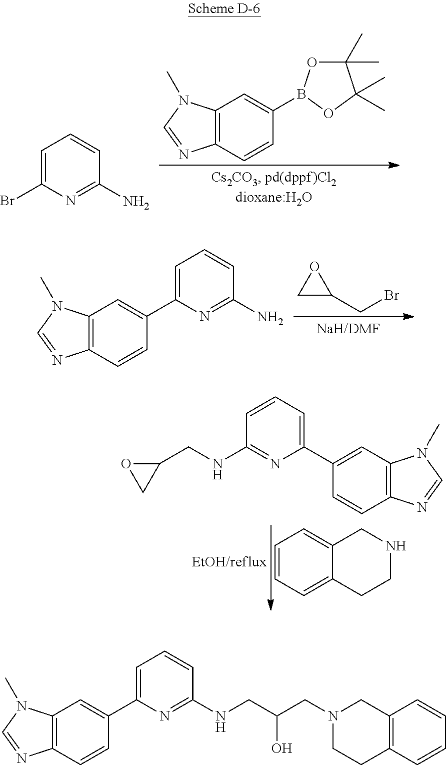

In some embodiments, compounds described herein can prepared using methods shown in general Scheme E-1 ring opening of a chiral or racemic epoxide group to form an amino alcohol moiety. A ring opening step can be performed in either direction as shown in scheme 1.

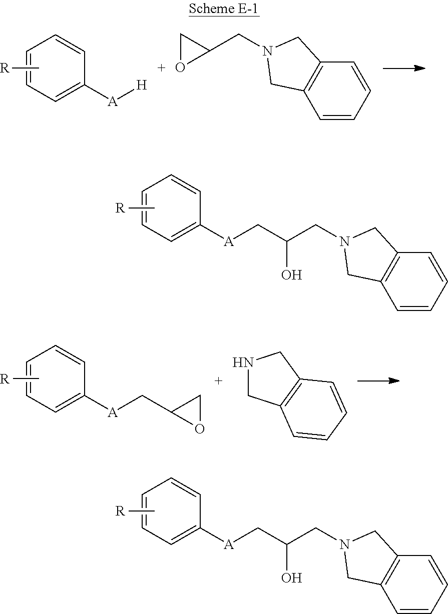

In some embodiments, compounds described herein can prepared using methods shown in general Scheme E-2. Compound B can be prepared via ring opening of a chiral or racemic epoxide group. This amino alcohol intermediate can be coupled to form an amide via normal amide coupling methodology using a carboxylic acid A wherein Z₁ is hydrogen or via amination of an ester of intermediate A when Z₁ is an optionally substituted aliphatic group.

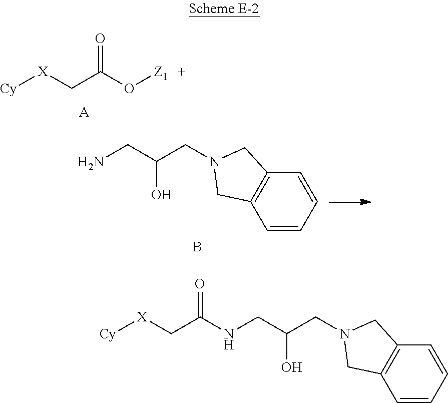

In some embodiments, compounds described herein can prepared using methods shown in general Scheme E-3. Compound Bc can be prepared via ring opening of a chiral or racemic epoxide group. This amino alcohol intermediate can be coupled to form an amide via normal amide coupling methodology using a carboxylic acid A wherein Z₁ is hydrogen or via amination of an ester of intermediate A when Z₁ is an optionally substituted aliphatic group

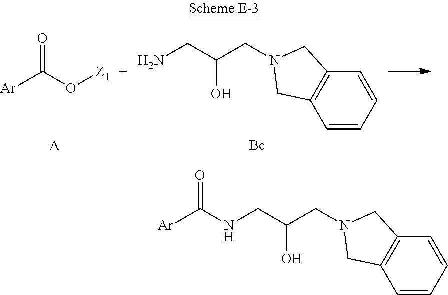

In some embodiments, compounds described herein can prepared using methods shown in general Scheme E-4, which describes ring opening of a chiral or racemic epoxide group to form the amino alcohol moiety linker.

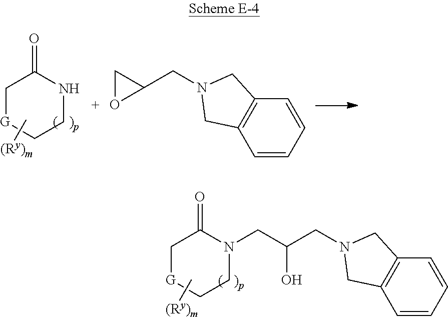

### EXAMPLES

In order that the invention described herein may be more fully understood, the following examples are set forth. It should be understood that these examples are for illustrative purposes only and are not to be construed as limiting this invention in any manner.

### PRMT5-MEP50 Crystallization and Structure Determination

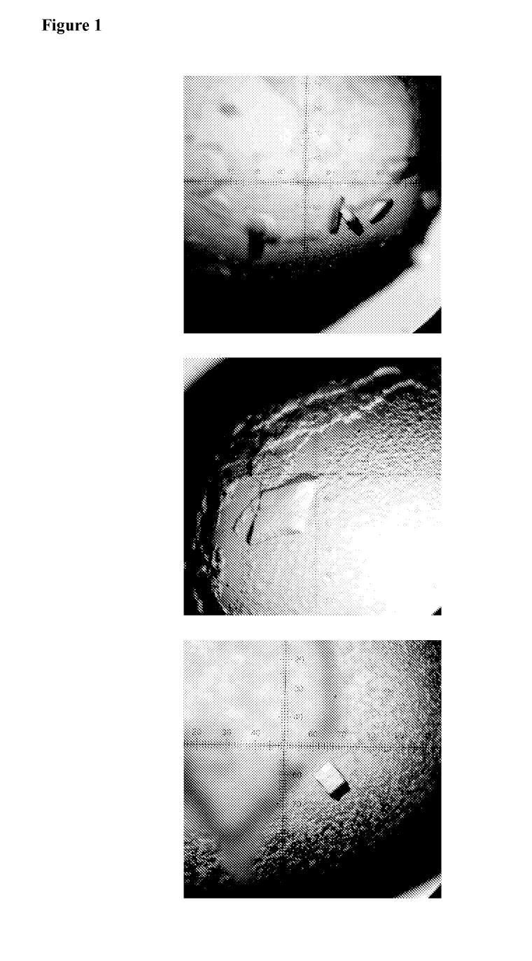

Crystals were grown using the coexpressed PRMT5-MEP50, stored in a buffer containing 50 mM Tris, 250 mM sodium chloride, 1 mM TCEP, pH 8.0 and concentrated to 10-30 mg/ml. The protein (typically at 15 mg/ml) was incubated with 0.5-2 mM sinefungin or SAM or SAH, and 0.5-2 mM compound (solubilized at 50-200 mM in DMSO) on ice for 20-120 minutes prior to crystallization. Crystals were grown using vapor diffusion methods with hanging drop trays. 1+1 μL or 2+2 μL drops were suspended over well solutions containing 0.05-0.5 M sodium acetate, 0.05-0.2 M sodium citrate pH 5-6, 5-15% w/v PEG 4000 (measured pH 5.7-6.5), with typical crystallization conditions being 0.2 M sodium acetate, 0.1 M sodium citrate pH 5.5, 10% w/v PEG 4000 (measured pH=6.1). Seeding techniques are used to reliably obtain crystals of protein-ligand complexes. Crystals appeared after 1-2 days and grew to full size in 5-7 days. A selection of PRMT5-MEP5-compound crystals is shown in FIG. 1. Crystals were cryoprotected into a final solution of 0.2 M sodium acetate, 0.1 M sodium citrate pH 5.5, 10% w/v PEG 4000, 20% glycerol through a series of step increases in glycerol concentration and flash frozen in liquid nitrogen prior to data collection.

Data collection was done at home x-ray sources or at synchrotron sources (ie, APS beamline 21-ID-F), with synchrotron sources being the preferred method of data acquisition. Typically, 180° of data were obtained using 0.5-1° oscillations per frame. Data reduction was done using a variety of programs, including but not limited to Xia2, HKL2000, d*TREK, XDS, MOSFLM, etc. Crystals usually belong to space group I222 with unit cell parameters a=103.6 Å, b=137.8 Å, c=178.8 Å, α=β=γ=90°; variation around these numbers is normal for individual datasets. Crystals also belonged to space group C2 with unit cell parameters a=225.7 Å, b=104.3 Å, c=138.0 Å, α=90°, β=127.5°, γ=90°. Scaling of data was done using Aimless, Scala, XSCALE, d*TREK, Scalepack, or other programs. In some cases, molecular replacement can be performed using a previously determined structure of PRMT5-MEP50-compound using AMoRe, Phaser, MolRep or other crystallography programs, or using difference Fourier methods. Once phases were determined and the ligand was placed in the active site as defined by the difference density observed, refinement of the final structure typically was done using REFMACS, but other programs such as BUSTER, CNX, PHENIX, etc can also be used.

Biological Assays

PRMT5 Biochemical Assay

General Materials.

S-adenosylmethionine (SAM), S-adenosylhomocysteine (SAH), bicine, KCl, Tween20, dimethylsulfoxide (DMSO), bovine skin gelatin (BSG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol 384-well streptavidin Flashplates were purchased from PerkinElmer.

Substrates.

Peptide representative of human histone H4 residues 1-15 was synthesized with a C-terminal linker-affinity tag motif and a C-terminal amide cap by 21^{st} Century Biochemicals. The peptide was high high-performance liquid chromatography (HPLC) purified to greater than 95% purity and confirmed by liquid chromatography mass spectrometry (LC-MS). The sequence was Ac-SGRGKGGKGLGKGGA[K-Biot]-amide (SEQ ID NO.:3).

Molecular Biology:

Full-length human PRMT5 (NM_{—}006109.3) transcript variant 1 clone was amplified from a fetal brain cDNA library, incorporating flanking 5′ sequence encoding a FLAG tag (MDYKDDDDK) (SEQ ID NO.:4) fused directly to Ala 2 of PRMT5. Full-length human MEP50 (NM_{—}024102) clone was amplified from a human testis cDNA library incorporating a 5′ sequence encoding a 6-histidine tag (MHHHHHH) (SEQ ID NO.:5) fused directly to Arg 2 of MEP50. The amplified genes were sublconed into pENTR/D/TEV (Life Technologies) and subsequently transferred by Gateway™ attL×attR recombination to pDEST8 baculvirus expression vector (Life Technologies).

Protein Expression.

Recombinant baculovirus and Baculovirus-Infected Insect Cells (BIIC) were generated according to Bac-to-Bac kit instructions (Life Technologies) and Wasilko, 2006, respectively. Protein over-expression was accomplished by infecting exponentially growing Spodoptera frugiperda (SF9) cell culture at 1.2×10⁶ cell/ml with a 5000 fold dilution of BIIC stock. Infections were carried out at 27° C. for 72 hours, harvested by centrifugation, and stored at −80° C. for purification.

Protein Purification.

Expressed full-length human Flag-PRMT5/6His-MeP50 protein complex was purified from cell paste by NiNTA agarose affinity chromatography after a five hour equilibration of the resin with buffer containing 50 mM Tris-HCL, pH 8.0, 25 mM NaCl, and 1 mM TCEP at 4° C., to minimize the adsorption of tubulin impurity by the resin. Flag-PRMT5/6His-MeP50 was eluted with 300 mM Imidazole in the same buffer. The purity of recovered protein was 87%. Reference: Wasilko, D. J. and S. E. Lee: “TIPS: titerless infected-cells preservation and scale-up” Bioprocess J., 5 (2006), pp. 29-32.

Predicted Translations:

Flag-PRMT5

(SEQ ID NO.: 6)

MDYKDDDDKA AMAVGGAGGS RVSSGRDLNC VPEIADTLGA
VAKQGFDFLC MPVFHPRFKR EFIQEPAKNR PGPQTRSDLL
LSGRDWNTLI VGKLSPWIRP DSKVEKIRRN SEAAMLQELN
FGAYLGLPAF LLPLNQEDNT NLARVLTNHI HTGHHSSMFW
MRVPLVAPED LRDDIIENAP TTHTEEYSGE EKTWMWWHNF
RTLCDYSKRI AVALEIGADL PSNHVIDRWL GEPIKAAILP
TSIFLTNKKG FPVLSKMHQR LIFRLLKLEV QFIITGTNHH
SEKEFCSYLQ YLEYLSQNRP PPNAYELFAK GYEDYLQSPL
QPLMDNLESQ TYEVFEKDPI KYSQYQQAIY KCLLDRVPEE
EKDTNVQVLM VLGAGRGPLV NASLRAAKQA DRRIKLYAVE
KNPNAVVTLE NWQFEEWGSQ VTVVSSDMRE WVAPEKADII
VSELLGSFAD NELSPECLDG AQHFLKDDGV SIPGEYTSFL
APISSSKLYN EVRACREKDR DPEAQFEMPY VVRLHNFHQL
SAPQPCFTFS HPNRDPMIDN NRYCTLEFPV EVNTVLHGFA
GYFETVLYQD ITLSIRPETH SPGMFSWFPI LFPIKQPITV
REGQTICVRF WRCSNSKKVW YEWAVTAPVC SAIHNPTGRS
YTIG L
6His-MEP50

(SEQ ID NO.: 7)

MHHHHHHRKE TPPPLVPPAA REWNLPPNAP ACMERQLEAA
RYRSDGALLL GASSLSGRCW AGSLWLFKDP CAAPNEGFCS
AGVQTEAGVA DLTWVGERGI LVASDSGAVE LWELDENETL
IVSKFCKYEH DDIVSTVSVL SSGTQAVSGS KDICIKVWDL
AQQVVLSSYR AHAAQVTCVA ASPHKDSVFL SCSEDNRILL
WDTRCPKPAS QIGCSAPGYL PTSLAWHPQQ SEVFVFGDEN
GTVSLVDTKS TSCVLSSAVH SQCVTGLVFS PHSVPFLASL
SEDCSLAVLD SSLSELFRSQ AHRDFVRDAT WSPLNHSLLT
TVGWDHQVVH HVVPTEPLPA PGPASVTE

### General Procedure for PRMT5/MEP50 Enzyme Assays on Peptide Substrates.

The assays were all performed in a buffer consisting of 20 mM Bicine (pH=7.6), 1 mM TCEP, 0.005% BSG, and 0.002% Tween20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT5/MEP50, was added to columns 11, 12, 23, 24, rows I-P for the minimum signal control. A cocktail (40 ul) containing the PRMT5/MEP50 enzyme and the peptide was added by Multidrop Combi (Thermo-Fisher). The compounds were allowed to incubate with PRMT5/MEP50 for 30 min at 25 degrees Celsius, then a cocktail (10 ul) containing ³H-SAM was added to initiate the reaction (final volume=51 ul). The final concentrations of the components were as follows: PRMT5/MEP50 was 4 nM, ³H-SAM was 75 nM, peptide was 40 nM, SAH in the minimum signal control wells was 100 uM, and the DMSO concentration was 1%. The assays were stopped by the addition of non-radioactive SAM (10 ul) to a final concentration of 600 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed three times with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

### % Inhibition Calculation

$$\% inh = 100 - ( \frac{{dpm}_{cmpd} - {dpm}_{min}}{{dpm}_{max} - {dpm}_{min}} ) \times 100$$

where dpm=disintegrations per minute, cmpd=signal in assay well, and min and max are the respective minimum and maximum signal controls.

### Four-Parameter 1050 Fit

$$Y = Bottom + \frac{( Top - Bottom )}{( 1 + {( \frac{X}{{IC}_{50}} )}^{Hill Coefficient}}$$

where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### Z-138 Methylation Assay

Z-138 suspension cells were purchased from ATCC (American Type Culture Collection, Manassas, Va.). RPMI/Glutamax medium, penicillin-streptomycin, heat inactivated fetal bovine serum, and D-PBS were purchased from Life Technologies, Grand Island, N.Y., USA. Odyssey blocking buffer, 800CW goat anti-rabbit IgG (H+L) antibody, and Licor Odyssey infrared scanner were purchased from Licor Biosciences, Lincoln, Nebr., USA. Symmetric di-methyl arginine antibody was purchased from EMD Millipore, Billerica, Mass., USA. 16% Paraformaldehyde was purchased from Electron Microscopy Sciences, Hatfield, Pa., USA.

Z-138 suspension cells were maintained in growth medium (RPMI 1640 supplemented with 10% v/v heat inactivated fetal bovine serum and 100 units/mL penicillin-streptomycin) and cultured at 37° C. under 5% CO₂.

Cell Treatment, in Cell Western (ICW) for Detection of Symmetric Di-Methyl Arginine and DNA Content.

Z-138 cells were seeded in assay medium at a concentration of 50,000 cells per mL to a 384-well cell culture plate with 50 μL per well. Compound (100 nL) from 384 well source plates was added directly to 384 well cell plate. Plates were incubated at 37° C., 5% CO₂ for 96 hours. After four days of incubation, 40 μL of cells from incubated plates were added to poly-D-lysine coated 384 well culture plates (BD Biosciences 356697). Plates were incubated at room temperature for 30 minutes then incubated at 37° C., 5% CO₂ for 5 hours. After the incubation, 40 μL per well of 8% paraformaldehyde in PBS (16% paraformaldahyde was diluted to 8% in PBS) was added to each plate and incubated for 30 minutes. Plates were transferred to a Biotek 405 plate washer and washed 5 times with 100 μL per well of wash buffer (1×PBS with 0.1% Triton X-100 (v/v)). Next 30 μL per well of Odyssey blocking buffer were added to each plate and incubated 1 hour at room temperature. Blocking buffer was removed and 20 μL per well of primary antibody was added (symmetric di-methyl arginine diluted 1:100 in Odyssey buffer with 0.1% Tween 20 (v/v)) and plates were incubated overnight (16 hours) at 4° C. Plates were washed 5 times with 100 μL per well of wash buffer. Next 20 μL per well of secondary antibody was added (1:200 800CW goat anti-rabbit IgG (H+L) antibody, 1:1000 DRAQS (Biostatus limited) in Odyssey buffer with 0.1% Tween 20 (v/v)) and incubated for 1 hour at room temperature. The plates were washed 5 times with 100 μL per well wash buffer then 1 time with 100 μL per well of water. Plates were allowed to dry at room temperature then imaged on the Licor Odyssey machine which measures integrated intensity at 700 nm and 800 nm wavelengths. Both 700 and 800 channels were scanned.

Calculations:

First, the ratio for each well was determined by:

$$( \frac{symmetric di \text{-} methyl Arginine 800 nm value}{DRAQ 5 \; 700 nm value} )$$

Each plate included fourteen control wells of DMSO only treatment (minimum inhibition) as well as fourteen control wells for maximum inhibition treated with 3 μM of a reference compound (Background wells). The average of the ratio values for each control type was calculated and used to determine the percent inhibition for each test well in the plate. Reference compound was serially diluted three-fold in DMSO for a total of nine test concentrations, beginning at 3 μM. Percent inhibition was determined and IC₅₀ curves were generated using triplicate wells per concentration of compound.

Percent Inhibition=100−

$$( ( \frac{\begin{matrix}( Individual Test Sample Ratio ) - \\ ( Background Avg Ratio )\end{matrix}}{\begin{matrix}( Minimum Inhibition Ratio ) - \\ ( Background Average Ratio )\end{matrix}} ) * 100 )$$

### Z-138 Proliferation Assay

Z-138 suspension cells were purchased from ATCC (American Type Culture Collection, Manassas, Va.). RPMI/Glutamax medium, penicillin-streptomycin, heat inactivated fetal bovine serum were purchased from Life Technologies, Grand Island, N.Y., USA. V-bottom polypropylene 384-well plates were purchased from Greiner Bio-One, Monroe, N.C., USA. Cell culture 384-well white opaque plates were purchased from Perkin Elmer, Waltham, Mass., USA. Cell-Titer Gb® was purchased from Promega Corporation, Madison, Wis., USA. SpectraMax M5 plate reader was purchased from Molecular Devices LLC, Sunnyvale, Calif., USA.

Z-138 suspension cells were maintained in growth medium (RPMI 1640 supplemented with 10% v/v heat inactivated fetal bovine serum and cultured at 37° C. under 5% CO₂. Under assay conditions, cells were incubated in assay medium (RPMI 1640 supplemented with 10% v/v heat inactivated fetal bovine serum and 100 units/mL penicillin-streptomycin) at 37° C. under 5% CO₂.

For the assessment of the effect of compounds on the proliferation of the Z-138 cell line, exponentially growing cells were plated in 384-well white opaque plates at a density of 10,000 cells/ml in a final volume of 50 μl of assay medium. A compound source plate was prepared by performing triplicate nine-point 3-fold serial dilutions in DMSO, beginning at 10 mM (final top concentration of compound in the assay was 20 μM and the DMSO was 0.2%). A 100 nL aliquot from the compound stock plate was added to its respective well in the cell plate. The 100% inhibition control consisted of cells treated with 200 nM final concentration of staurosporine and the 0% inhibition control consisted of DMSO treated cells. After addition of compounds, assay plates were incubated for 5 days at 37° C., 5% CO₂, relative humidity >90%.

Cell viability was measured by quantitation of ATP present in the cell cultures, adding 35 μl of Cell Titer Glo® reagent to the cell plates. Luminescence was read in the SpectraMax M5 microplate reader. The concentration of compound inhibiting cell viability by 50% was determined using a 4-parametric fit of the normalized dose response curves.

Results for certain compounds described herein are shown in Table A-2.

TABLE A-2
Biological Assay Results

| Cmpd No | Biochemical IC₅₀ | ICW EC₅₀ | Proliferation EC₅₀ |
|---|---|---|---|
| A1 | B | B | — |
| A2 | C | — | — |
| A3 | C | — | — |
| A4 | A | — | — |
| A5 | D | — | — |
| A6 | A | A | B |
| A7 | B | B | D |
| A8 | B | B | D |
| A9 | B | D | D |
| A10 | C | — | — |
| A11 | B | B | D |
| A12 | B | B | D |
| A13 | C | — | — |
| A14 | C | — | — |
| A15 | B | B | C |
| A16 | B | B | D |
| A17 | B | B | D |
| A18 | C | C | ** |
| A19 | A | B | C |
| A20 | A | B | C |
| A21 | B | B | ** |
| A22 | A | B | C |
| A23 | A | B | C |
| A24 | B | — | — |
| A25 | B | — | — |
| A26 | A | B | C |
| A27 | A | B | C |
| A28 | A | B | C |
| A29 | B | B | ** |
| A30 | B | B | D |
| A31 | C | B | D |
| A32 | B | B | D |
| A33 | C | — | — |
| A34 | B | B | D |
| A35 | B | B | D |
| A36 | B | B | ** |
| A37 | A | A | C |
| A38 | A | A | C |
| A39 | A | A | B |
| A40 | A | B | C |
| A41 | C | — | — |
| A42 | B | B | ** |
| A43 | A | B | C |
| A44 | B | B | D |
| A45 | A | A | B |
| A46 | B | B | D |
| A47 | A | B | C |
| A48 | A | B | D |
| A49 | A | B | C |
| A50 | A | A | C |
| A51 | D | — | — |
| A52 | C | — | — |
| A53 | A | B | C |
| A54 | B | B | — |
| A55 | B | — | — |
| A56 | C | — | — |
| A57 | D | — | — |
| A58 | D | — | — |
| A59 | C | — | — |
| A60 | B | C | — |
| A61 | C | — | — |
| A62 | C | — | — |
| A63 | D | — | — |
| A64 | A | B | C |
| A65 | A | B | C |
| A66 | A | B | C |
| A67 | A | A | C |
| A68 | A | B | ** |
| A69 | B | C | — |
| A70 | A | B | ** |
| A71 | A | B | ** |
| A72 | C | — | — |
| A73 | A | A | B |
| A74 | A | B | C |
| A75 | A | A | C |
| A76 | A | B | C |
| A77 | A | A | C |
| A78 | B | B | — |
| A79 | A | B | C |
| A80 | A | B | D |
| A81 | A | A | B |
| A82 | A | A | C |
| A83 | B | B | D |
| A84 | A | B | C |
| A85 | C | C | — |
| A86 | A | B | D |
| A87 | C | — | — |
| A88 | A | B | D |
| A89 | B | C | — |
| A90 | A | B | D |
| A91 | A | B | C |
| A92 | A | A | C |
| A93 | A | A | C |
| A94 | A | B | D |
| A95 | A | B | D |
| A96 | A | B | D |
| A97 | B | B | C |
| A98 | A | A | C |
| A99 | A | B | C |
| A100 | A | A | C |
| A101 | A | A | C |
| A102 | A | A | C |
| A103 | A | B | ** |
| A104 | B | C | ** |
| A105 | A | B | C |
| A106 | B | B | ** |
| A107 | A | A | C |
| A108 | A | B | D |
| A109 | A | A | B |
| A110 | A | A | B |
| A111 | A | A | B |
| A112 | B | B | ** |
| A113 | B | B | D |
| A114 | B | C | ** |
| A115 | D | — | — |
| A116 | C | C | ** |
| A117 | B | B | C |
| A118 | B | C | ** |
| A119 | A | B | D |
| A120 | B | C | ** |
| A121 | C | — | — |
| A122 | B | C | ** |
| A123 | A | B | C |
| A124 | C | C | ** |
| A125 | C | — | — |
| A126 | E | — | — |
| A127 | B | C | ** |
| A128 | E | — | — |
| A129 | B | C | ** |
| A130 | A | B | C |
| A131 | C | — | — |
| A132 | C | — | — |
| A133 | * | — | — |
| A134 | B | C | ** |
| A135 | C | — | — |
| A136 | C | — | — |
| A137 | B | C | ** |
| A138 | B | C | — |
| A139 | * | — | — |
| A140 | C | — | — |
| A141 | C | — | — |
| A142 | B | B | ** |
| A143 | C | — | — |
| A144 | * | — | — |
| A145 | C | — | — |
| A146 | A | B | — |
| A147 | * | — | — |
| A148 | * | — | — |
| A149 | A | B | — |
| A150 | B | — | — |
| A151 | B | — | — |
| A152 | C | — | — |

For Table A-2, “A” indicates an IC₅₀ or EC₅₀ < 0.100 μM, “B” indicates an IC₅₀ or EC₅₀ of 0.101-1.000 μM, “C” indicates an IC₅₀ or EC₅₀ of 1.001-10.000 μM, “D” indicates an IC₅₀ or EC₅₀ of 10.001-50 μM, and “E” indicates an IC₅₀ or EC₅₀ > 50 μM.
“—” indicates no data shown.
“*” indicates an IC₅₀ or EC₅₀ > 10 μM.
“**” indicates an IC₅₀ or EC₅₀ > 20 μM

Results for certain compounds described herein are shown in Table B-2.

TABLE B-2
Biological Assay Results

| Cmpd No | Biochemical IC₅₀ | ICW EC₅₀ | Proliferation EC₅₀ |
|---|---|---|---|
| B1. | A | A | C |
| B2. | A | A | C |
| B3. | A | A | C |
| B4. | C | — | — |
| B5. | B | B | ** |
| B6. | C | — | — |
| B7. | C | — | — |
| B8. | A | A | C |
| B9. | A | A | C |
| B10. | A | B | C |
| B11. | A | C | — |
| B12. | B | C | ** |
| B13. | A | A | B |
| B14. | A | B | C |
| B15. | A | B | D |
| B16. | A | A | B |
| B17. | B | B | ** |
| B18. | B | B | D |
| B19. | A | B | D |
| B20. | A | A | B |
| B21. | B | B | ** |
| B22. | B | B | ** |
| B23. | B | B | ** |
| B24. | A | B | C |
| B25. | B | C | ** |
| B26. | B | B | D |
| B27. | C | — | — |
| B28. | A | B | D |
| B29. | A | B | C |
| B30. | A | B | C |
| B31. | B | B | D |
| B32. | B | B | ** |
| B33. | C | — | — |
| B34. | A | B | D |
| B35. | A | B | D |
| B36. | A | B | D |
| B37. | A | B | ** |
| B38. | A | B | D |
| B39. | B | C | ** |
| B40. | A | A | C |
| B41. | A | A | C |
| B42. | B | C | ** |
| B43. | B | B | C |
| B44. | A | — | B |
| B45. | A | B | — |
| B46. | C | — | — |
| B47. | B | B | — |
| B48. | B | B | — |
| B49. | B | — | — |
| B50. | B | — | — |
| B51. | B | — | — |
| B52. | B | — | — |
| B53. | B | — | — |
| B54. | A | — | — |
| B55. | A | — | — |
| B56. | B | — | — |
| B57. | B | — | — |
| B58. | A | — | — |

For Table B-2, “A” indicates an IC₅₀ or EC₅₀ < 0.100 μM, “B” indicates an IC₅₀ or EC₅₀ of 0.101-1.000 μM, “C” indicates an IC₅₀ or EC₅₀ of 1.001-10.000 μM, “D” indicates an IC₅₀ or EC₅₀ of 10.001-50 μM, and “E” indicates an IC₅₀ or EC₅₀ > 50 μM.
“—” indicates no data.
“**” indicates an IC₅₀ or EC₅₀ > 20 μM.

Results for certain compounds described herein are shown in Table C-2.

TABLE C-2
Biological Assay Results

| Cmpd No | Biochemical IC₅₀ | ICW EC₅₀ | Proliferation EC₅₀ |
|---|---|---|---|
| C1 | B | B | ** |
| C2 | D | — | — |
| C3 | D | — | — |
| C4 | A | B | C |
| C5 | A | C | — |
| C6 | A | B | ** |
| C7 | A | B | D |
| C8 | A | B | C |
| C9 | B | B | D |
| C10 | — | E | — |
| C11 | — | E | — |
| C12 | A | A | C |
| C13 | — | E | — |
| C14 | — | E | — |
| C15 | B | B | ** |
| C16 | C | — | — |
| C17 | C | — | — |
| C18 | C | — | — |
| C19 | B | B | ** |
| C20 | B | C | — |
| C21 | C | — | — |
| C22 | C | — | — |
| C23 | C | — | — |
| C24 | C | — | — |
| C25 | C | — | — |
| C26 | C | — | — |
| C27 | E | — | — |
| C28 | C | — | — |
| C29 | B | B | D |
| C30 | C | — | — |
| C31 | B | B | ** |
| C32 | * | — | — |

For Table C-2, “A” indicates an IC₅₀ or EC₅₀ < 0.100 μM, “B” indicates an IC₅₀ or EC₅₀ of 0.101-1.000 μM, “C” indicates an IC₅₀ or EC₅₀ of 1.001-10.000 μM, “D” indicates an IC₅₀ or EC₅₀ of 10.001-50 μM, and “E” indicates an IC₅₀ or EC₅₀ > 50 μM.
“—” indicates no data.
“*” indicates an IC₅₀ or EC₅₀ > 10 μM.
“**” indicates an IC₅₀ or EC₅₀ > 20 μM.

Results for certain compounds described herein are shown in Table D-2.

TABLE D-2
Biological Assay Results

| Cmpd No | Biochemical IC₅₀ | ICW EC₅₀ | Proliferation EC₅₀ |
|---|---|---|---|
| D1. | B | — | D |
| D2. | C | — | — |
| D3. | B | — | — |
| D4. | B | — | — |
| D5. | B | — | — |
| D6. | B | — | — |
| D7. | D | — | — |
| D8. | C | — | — |
| D9. | C | — | ** |
| D10. | B | — | — |
| D11. | B | — | — |
| D12. | C | — | ** |
| D13. | C | — | — |
| D14. | C | — | — |
| D15. | C | — | — |
| D16. | C | — | — |
| D17. | B | — | — |
| D18. | C | — | ** |
| D19. | C | — | — |
| D20. | C | — | — |
| D21. | C | — | E |
| D22. | B | — | D |
| D23. | C | — | D |
| D24. | C | — | E |
| D25. | C | — | D |
| D26. | D | — | E |
| D27. | C | — | E |
| D28. | C | — | E |
| D29. | D | — | D |
| D30. | E | — | D |
| D31. | B | C | D |
| D32. | C | ** | E |
| D33. | C | — | E |
| D34. | C | — | D |
| D35. | C | — | — |
| D36. | D | — | — |
| D37. | C | — | — |
| D38. | E | — | — |
| D39. | B | C | ** |
| D40. | C | — | — |
| D41. | C | — | — |
| D42. | D | — | — |
| D43. | B | C | ** |
| D44. | C | — | — |
| D45. | A | B | C |
| D46. | C | — | — |
| D47. | A | B | C |
| D48. | C | — | — |
| D49. | B | B | C |
| D50. | C | — | — |
| D51. | E | — | — |
| D52. | C | — | — |
| D53. | B | B | D |
| D54. | E | — | — |
| D55. | C | — | — |
| D56. | E | — | — |
| D57. | C | — | — |
| D58. | B | C | D |
| D59. | B | D | D |
| D60. | B | B | D |
| D61. | E | — | — |
| D62. | B | C | D |
| D63. | B | C | D |
| D64. | C | — | — |
| D65. | A | B | D |
| D66. | B | — | — |
| D67. | B | C | D |
| D68. | B | D | D |
| D69. | C | — | — |
| D70. | C | — | — |
| D71. | C | — | — |
| D72. | B | C | D |
| D73. | C | — | — |
| D74. | B | C | D |
| D75. | B | C | D |
| D76. | C | — | — |
| D77. | C | — | — |
| D78. | C | — | — |
| D79. | D | — | — |
| D80. | B | ** | C |
| D81. | B | B | D |
| D82. | B | C | D |
| D83. | B | C | D |
| D84. | B | C | C |
| D85. | B | C | D |
| D86. | B | C | D |
| D87. | C | — | — |
| D88. | B | C | D |
| D89. | B | C | D |
| D90. | B | C | D |
| D91. | C | — | — |
| D92. | B | C | D |
| D93. | B | C | D |
| D94. | B | C | D |
| D95. | B | C | D |
| D96. | C | — | — |
| D97. | C | — | — |
| D98. | C | — | — |
| D99. | D | — | — |
| D100. | C | — | — |
| D101. | C | — | — |
| D102. | A | B | C |
| D103. | B | B | C |
| D104. | B | C | ** |
| D105. | A | C | D |
| D106. | A | C | ** |
| D107. | B | C | ** |
| D108. | A | C | D |
| D109. | B | B | D |
| D110. | B | ** | — |
| D111. | B | C | — |
| D112. | B | ** | ** |
| D113. | B | B | D |
| D114. | B | ** | — |
| D115. | C | — | — |
| D116. | A | C | ** |
| D117. | B | C | ** |
| D118. | A | B | C |
| D119. | B | B | C |
| D120. | C | — | — |
| D121. | B | B | ** |
| D122. | C | — | — |
| D123. | B | C | D |
| D124. | B | D | ** |
| D125. | B | C | D |
| D126. | B | C | ** |
| D127. | B | D | ** |
| D128. | C | — | — |
| D129. | B | C | D |
| D130. | B | C | ** |
| D131. | B | C | ** |
| D132. | B | C | ** |
| D133. | C | — | — |
| D134. | B | C | ** |
| D135. | B | ** | ** |
| D136. | B | C | ** |
| D137. | B | C | ** |
| D138. | B | C | ** |
| D139. | B | C | ** |
| D140. | C | — | — |
| D141. | A | B | ** |
| D142. | C | — | — |
| D143. | E | — | — |
| D144. | C | — | — |
| D145. | B | C | ** |
| D146. | E | — | — |
| D147. | B | C | D |
| D148. | B | C | D |
| D149. | B | C | C |
| D150. | B | C | ** |
| D151. | B | C | ** |
| D152. | B | C | ** |
| D153. | C | — | — |
| D154. | B | C | — |
| D155. | C | — | — |
| D156. | B | C | D |
| D157. | C | — | — |
| D158. | C | — | — |
| D159. | C | — | — |
| D160. | C | C | ** |
| D161. | C | C | ** |
| D162. | C | C | ** |
| D163. | C | C | — |
| D164. | C | C | — |
| D165. | C | — | — |
| D166. | C | D | ** |
| D167. | B | C | ** |
| D168. | B | D | ** |
| D169. | B | B | ** |
| D170. | B | C | ** |
| D171. | B | C | ** |
| D172. | B | C | ** |
| D173. | B | C | C |
| D174. | A | B | C |
| D175. | C | — | — |
| D176. | A | B | ** |
| D177. | D | — | — |
| D178. | E | — | — |
| D179. | B | — | ** |
| D180. | C | ** | ** |
| D181. | C | — | — |
| D182. | C | C | ** |
| D183. | C | C | ** |
| D184. | C | — | — |
| D185. | B | C | D |
| D186. | B | C | ** |
| D187. | B | C | ** |
| D188. | B | C | D |
| D189. | A | B | B |
| D190. | C | — | — |
| D191. | B | B | C |
| D192. | C | — | — |
| D193. | E | — | — |
| D194. | C | — | — |
| D195. | C | — | — |
| D196. | C | — | — |
| D197. | C | — | — |
| D198. | D | — | — |
| D199. | A | A | C |
| D200. | B | D | ** |
| D201. | B | C | ** |
| D202. | E | — | — |
| D203. | C | — | — |
| D204. | C | — | — |
| D205. | B | B | ** |
| D206. | C | — | — |
| D207. | B | B | C |
| D208. | B | C | C |
| D209. | A | B | C |
| D210. | A | B | D |
| D211. | C | — | — |
| D212. | C | — | — |
| D213. | C | — | — |
| D214. | C | — | — |
| D215. | C | — | — |
| D216. | B | C | ** |
| D217. | B | C | D |
| D218. | A | B | C |
| D219. | A | B | C |
| D220. | A | B | C |
| D221. | A | B | C |
| D222. | B | B | D |
| D223. | A | B | D |
| D224. | B | B | ** |
| D225. | E | — | — |
| D226. | A | B | C |
| D227. | A | — | — |
| D228. | B | — | — |
| D229. | A | — | — |
| D230. | A | — | — |
| D231. | B | — | — |
| D232. | B | — | — |
| D233. | A | A | C |
| D234. | A | A | B |
| D235. | B | B | D |
| D236. | A | B | C |
| D237. | B | B | D |
| D238. | A | A | C |
| D239. | A | A | C |
| D240. | A | B | C |
| D241. | A | B | C |
| D242. | B | B | C |
| D243. | B | B | C |
| D244. | B | B | D |
| D245. | A | B | C |
| D246. | A | A | B |
| D247. | A | A | C |
| D248. | B | B | D |
| D249. | B | B | D |
| D250. | A | B | C |
| D251. | A | B | C |
| D252. | A | B | C |
| D253. | A | B | C |
| D254. | A | B | C |
| D255. | A | A | B |
| D256. | A | A | B |
| D257. | A | B | C |
| D258. | B | C | ** |
| D259. | B | B | D |
| D260. | C | — | — |
| D261. | A | B | D |
| D262. | B | C | C |
| D263. | B | B | D |
| D264. | B | C | C |
| D265. | A | B | C |
| D266. | A | C | C |
| D267. | B | — | — |
| D268. | C | — | — |
| D269. | A | B | D |
| D270. | B | B | D |
| D271. | B | B | ** |
| D272. | B | B | C |
| D273. | A | B | D |
| D274. | C | — | — |
| D275. | A | B | C |
| D276. | B | C | C |
| D277. | * | — | — |
| D278. | C | — | — |
| D279. | C | — | — |
| D280. | C | — | D |
| D281. | C | — | — |
| D282. | D | — | — |
| D283. | D | — | — |
| D284. | D | — | — |
| D285. | C | — | ** |
| D286. | B | — | ** |
| D287. | C | — | — |
| D288. | C | — | E |
| D289. | E | — | E |
| D290. | C | — | E |
| D291. | C | — | E |
| D292. | C | — | E |
| D293. | E | — | E |
| D294. | C | — | E |
| D295. | E | — | E |
| D296. | E | — | — |
| D297. | E | — | E |
| D298. | E | — | E |
| D299. | D | — | E |
| D300. | B | — | E |
| D301. | C | — | E |
| D302. | C | — | — |
| D303. | C | — | E |
| D304. | E | — | E |
| D305. | D | — | E |
| D306. | E | — | E |
| D307. | E | — | E |
| D308. | D | — | E |
| D309. | C | — | E |
| D310. | D | — | E |

For Table D-2, “A” indicates an IC₅₀ or EC₅₀ < 0.100 μM, “B” indicates an IC₅₀ or EC₅₀ of 0.101-1.000 μM, “C” indicates an IC₅₀ or EC₅₀ of 1.001-10.000 μM, “D” indicates an IC₅₀ or EC₅₀ of 10.001-40 μM, and “E” indicates an IC₅₀ or EC₅₀ > 40 μM.
“—” indicates no data shown.
“*” indicates an IC₅₀ or EC₅₀ > 10 μM.
“**” indicates an IC₅₀ or EC₅₀ > 20 μM.

Results for certain compounds described herein are shown in Table E-2.

TABLE E-2
Biological Assay Results

| Cmpd No | Biochemical IC₅₀ | ICW EC₅₀ | Proliferation EC₅₀ |
|---|---|---|---|
| E1. | C | — | — |
| E2. | C | — | — |
| E3. | C | — | — |
| E4. | A | B | C |
| E5. | A | B | C |
| E6. | * | — | — |
| E7. | B | B | ** |
| E8. | B | B | — |
| E9. | C | — | — |
| E10. | B | B | ** |
| E11. | B | C | — |
| E12. | B | B | — |
| E13. | B | C | ** |
| E14. | D | — | — |
| E15. | A | B | C |
| E16. | C | — | — |
| E17. | C | — | — |
| E18. | C | — | — |
| E19. | B | C | ** |
| E20. | C | — | D |

For Table E-2, “A” indicates an IC₅₀ or EC₅₀ < 0.100 μM, “B” indicates an IC₅₀ or EC₅₀ of 0.101-1.000 μM, “C” indicates an IC₅₀ or EC₅₀ of 1.001-10.000 μM, “D” indicates an IC₅₀ or EC₅₀ of 10.001-50 μM, and “E” indicates an IC₅₀ or EC₅₀ > 50 μM.
“—” indicates no data shown.
“*” indicates an IC₅₀ or EC₅₀ > 10 μM.
“**” indicates an IC₅₀ or EC₅₀ > 20 μM.

### Other Embodiments

The foregoing has been a description of certain non-limiting embodiments of the invention. Those of ordinary skill in the art will appreciate that various changes and modifications to this description may be made without departing from the spirit or scope of the present invention, as defined in the following claims.

TABLE A
Untitled

| HEADER | ---- | XX-XXX-XX XXXX |
|---|---|---|
| COMPND | --- | |
| REMARK | 3 | |

| | | | |
|---|---|---|---|
| REMARK | 3 | REFINEMENT. | |
| REMARK | 3 | PROGRAM | : REFMAC 5.7.0029 |
| REMARK | 3 | AUTHORS | : NURSHUDOV, SKUBAK, LEBEDEV, PANNU, |
| REMARK | 3 | | STEINER, NICHOLLS, WINN, LONG, VAGIN |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 2.39 |
| REMARK | 3 | RESOLUTION RANGE LOW | (ANGSTROMS) | : 41.37 |
| REMARK | 3 | DATA CUTOFF | (SIGMA(F)) | : NONE |
| REMARK | 3 | COMPLETENESS FOR RANGE | (%) | : 99.59 |
| REMARK | 3 | NUMBER OF REFLECTIONS | | : 48207 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.21203 |
| REMARK | 3 | R VALUE | (WORKING SET) | : 0.20841 |

| REMARK | 3 | FREE R VALUE | | : 0.28210 |
|---|---|---|---|---|
| REMARK | 3 | FREE R VALUE TEST SET SIZE | (%) | : 5.1 |

| | | | |
|---|---|---|---|
| REMARK | 3 | FREE R VALUE TEST SET COUNT | : 2579 |
| REMARK | 3 | | |

| REMARK | 3 | FIT IN THE HIGHEST RESOLUTION BIN. | | | |
|---|---|---|---|---|---|
| REMARK | 3 | TOTAL NUMBER OF BINS USED | | : | 20 |
| REMARK | 3 | BIN RESOLUTION RANGE HIGH | | : | 2.390 |
| REMARK | 3 | BIN RESOLUTION RANGE LOW | | : | 2.452 |
| REMARK | 3 | REFLECTION IN BIN | (WORKING SET) | : | 3459 |
| REMARK | 3 | BIN COMPLETENESS | (WORKING + TEST) (%) | : | 98.89 |
| REMARK | 3 | BIN R VALUE | (WORKING SET) | : | 0.360 |

| | | | | |
|---|---|---|---|---|
| REMARK | 3 | BIN FREE R VALUE SET COUNT | : | 202 |
| REMARK | 3 | BIN FREE R VALUE | : | 0.426 |
| REMARK | 3 | | | |

| REMARK | 3 | NUMBER OF NON-HYDROGEN ATOMS USED IN REFINEMENT. | | |
|---|---|---|---|---|
| REMARK | 3 | ALL ATOMS | : | 7699 |
| REMARK | 3 | | | |
| REMARK | 3 | B VALUES. | | |

| | | | | |
|---|---|---|---|---|
| REMARK | 3 | FROM WILSON PLOT | (A**2) | : NULL |
| REMARK | 3 | MEAN B VALUE | (OVERALL, A**2) | : 52.795 |

| REMARK | 3 | OVERALL ANISOTROPIC B VALUE. | |
|---|---|---|---|
| REMARK | 3 | B11 (A**2) : | 0.25 |
| REMARK | 3 | B22 (A**2) : | −0.06 |
| REMARK | 3 | B33 (A**2) : | −0.19 |
| REMARK | 3 | B12 (A**2) : | 0.00 |
| REMARK | 3 | B13 (A**2) : | 0.00 |
| REMARK | 3 | R23 (A**2) : | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A): | 0.338 |
| REMARK | 3 | ESU BASED ON FREE R VALUE | (A): | 0.275 |
| REMARK | 3 | ESU BASED ON MAXIMUM LIKELIHOOD | (A): | 0.233 |
| REMARK | 3 | ESU FOR B VALUES BASED ON MAXIMUM LIKELIHOOD | (A**2): | 10.464 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.952 |
| REMARK | 3 | CORRELATION COEFFICIENT FO-FC FREE | : 0.910 |
| REMARK | 3 | | |

| | | | | | | |
|---|---|---|---|---|---|---|
| REMARK | 3 | RMS DEVIATIONS FROM IDEAL VALUES | | COUNT | RMS | WEIGHT |
| REMARK | 3 | BOND LENGTHS REFINED ATOMS | (A): | 7771; | 0.009; | 0.019 |
| REMARK | 3 | BOND ANGLES REFINED ATOMS | (DEGREES): | 10600; | 1.413; | 1.958 |
| REMARK | 3 | TORSION ANGLES, PERIOD 1 | (DEGREES): | 952; | 6.919; | 5.000 |
| REMARK | 3 | TORSION ANGLES, PERIOD 2 | (DEGREES): | 356; | 35.525; | 24.017 |
| REMARK | 3 | TORSION ANGLES, PERIOD 3 | (DEGREES): | 1244; | 18.128; | 15.000 |
| REMARK | 3 | TORSION ANGLES, PERIOD 4 | (DEGREES): | 47; | 19.255; | 15.000 |
| REMARK | 3 | CHIRAL-CENTER RESTRAINTS | (A**3): | 1163; | 0.093; | 0.200 |
| REMARK | 3 | GENERAL PLANES REFINED ATOMS | (A): | 5985; | 0.006; | 0.021 |
| REMARK | 3 | | | | | |

| | | | | | |
|---|---|---|---|---|---|
| REMARK | 3 | ISOTROPIC THERMAL FACTOR RESTRAINTS. | COUNT | RMS | WEIGHT |
| REMARK | 3 | | | | |
| REMARK | 3 | NCS RESTRAINTS STATISTICS | | | |
| REMARK | 3 | NUMBER OF NCS GROUPS : NULL | | | |
| REMARK | 3 | | | | |
| REMARK | 3 | TWIN DETAILS | | | |
| REMARK | 3 | NUMBER OF TWIN DOMAINS : NULL | | | |
| REMARK | 3 | | | | |
| REMARK | 3 | | | | |
| REMARK | 3 | TLS DETAILS | | | |
| REMARK | 3 | NUMBER OF TLS GROUPS : NULL | | | |
| REMARK | 3 | | | | |
| REMARK | 3 | | | | |
| REMARK | 3 | BULK SOLVENT MODELLING. | | | |
| REMARK | 3 | METHOD USED : MASK | | | |
| REMARK | 3 | PARAMETERS FOR MASK CALCULATION | | | |

| | | | | |
|---|---|---|---|---|
| REMARK | 3 | VDW PROBE RADIUS | : | 1.20 |
| REMARK | 3 | ION PROBE RADIUS | : | 0.80 |
| REMARK | 3 | SHRINKAGE RADIUS | : | 0.80 |
| REMARK | 3 | | | |

| REMARK | 3 | OTHER REFINEMENT REMARKS: | | | | | | |
|---|---|---|---|---|---|---|---|---|
| REMARK | 3 | HYDROGENS HAVE BEEN USED IF PRESENT IN THE INPUT | | | | | | |
| REMARK | 3 | U VALUES : REFINED INDIVIDUALLY | | | | | | |
| REMARK | 3 | | | | | | | |
| SSBOND | 1 | CYS | B | 247 | CYS | B | 208 | |
| CISPEP | 1 | GLU | A | 57 | PRO | A | 58 | 0.00 |
| CISPEP | 2 | GLY | A | 64 | PRO | A | 65 | 0.00 |
| CISPEP | 3 | ILE | A | 464 | PRO | A | 465 | 0.00 |
| CISPEP | 4 | HIS | A | 523 | PRO | A | 524 | 0.00 |
| CISPEP | 5 | ALA | A | 619 | PRO | A | 620 | 0.00 |
| CISPEP | 6 | ALA | B | 67 | PRO | B | 68 | 0.00 |

| CRYST1 | 103.400 138.130 178.920 90.00 90.00 90.00 | | | | | | I 2 2 2 | | | | |
|---|---|---|---|---|---|---|---|---|---|---|---|
| SCALE1 | 0.009671 0.000000 0.000000 0.00000 | | | | | | | | | | |
| SCALE2 | 0.000000 0.007740 0.000000 0.00000 | | | | | | | | | | |
| SCALE3 | 0.000000 0.000000 0.005589 0.00000 | | | | | | | | | | |
| ATOM | 1 | N | ARG | A | 13 | 40.231 | −69.664 | −17.513 | 1.00 | 90.11 | N |
| ATOM | 2 | CA | ARG | A | 13 | 40.367 | −69.723 | −19.001 | 1.00 | 91.57 | C |
| ATOM | 3 | CB | ARG | A | 13 | 41.647 | −70.468 | −19.418 | 1.00 | 99.54 | C |
| ATOM | 4 | CG | ARG | A | 13 | 42.824 | −70.300 | −18.469 | 1.00 | 103.69 | C |
| ATOM | 5 | CD | ARG | A | 13 | 42.870 | −71.403 | −17.419 | 1.00 | 102.49 | C |
| ATOM | 6 | NE | ARG | A | 13 | 43.736 | −72.505 | −17.836 | 1.00 | 115.87 | N |
| ATOM | 7 | CZ | ARG | A | 13 | 45.053 | −72.561 | −17.614 | 1.00 | 123.49 | C |
| ATOM | 8 | NH1 | ARG | A | 13 | 45.759 | −73.508 | −18.035 | 1.00 | 121.07 | N |
| ATOM | 9 | NH2 | ARG | A | 13 | 45.674 | −71.573 | −16.974 | 1.00 | 127.94 | N |
| ATOM | 10 | C | ARG | A | 13 | 39.140 | −70.424 | −19.570 | 1.00 | 85.13 | C |
| ATOM | 11 | O | ARG | A | 13 | 38.027 | −69.906 | −19.476 | 1.00 | 82.98 | O |
| ATOM | 12 | N | VAL | A | 14 | 39.341 | −71.506 | −20.143 | 1.00 | 74.74 | N |
| ATOM | 13 | CA | VAL | A | 14 | 38.239 | −72.385 | −20.680 | 1.00 | 66.66 | C |
| ATOM | 14 | CB | VAL | A | 14 | 38.466 | −72.722 | −22.162 | 1.00 | 63.40 | C |
| ATOM | 15 | CG1 | VAL | A | 14 | 37.403 | −73.583 | −22.669 | 1.00 | 63.40 | C |
| ATOM | 16 | CG2 | VAL | A | 14 | 38.441 | −71.457 | −22.979 | 1.00 | 62.45 | C |
| ATOM | 17 | C | VAL | A | 14 | 38.057 | −73.676 | −19.897 | 1.00 | 63.08 | C |
| ATOM | 18 | O | VAL | A | 14 | 38.982 | −74.482 | −19.802 | 1.00 | 64.30 | O |
| ATOM | 19 | N | SER | A | 15 | 36.864 | −73.858 | −19.338 | 1.00 | 54.68 | N |
| ATOM | 20 | CA | SER | A | 15 | 36.473 | −75.135 | −18.760 | 1.00 | 51.93 | C |
| ATOM | 21 | CB | SER | A | 15 | 35.403 | −74.956 | −17.687 | 1.00 | 52.49 | C |
| ATOM | 22 | OG | SER | A | 15 | 35.360 | −76.087 | −16.844 | 1.00 | 54.48 | O |
| ATOM | 23 | C | SER | A | 15 | 35.964 | −76.025 | −19.879 | 1.00 | 49.02 | C |
| ATOM | 24 | O | SER | A | 15 | 35.121 | −75.622 | −20.680 | 1.00 | 50.25 | O |
| ATOM | 25 | N | SER | A | 16 | 36.489 | −77.235 | −19.934 | 1.00 | 46.39 | N |
| ATOM | 26 | CA | SER | A | 16 | 36.263 | −78.108 | −21.059 | 1.00 | 46.79 | C |
| ATOM | 27 | CB | SER | A | 16 | 37.373 | −77.904 | −22.067 | 1.00 | 54.99 | C |
| ATOM | 28 | OG | SER | A | 16 | 38.597 | −78.114 | −21.491 | 1.00 | 65.11 | O |
| ATOM | 29 | C | SER | A | 16 | 36.271 | −75.548 | −20.593 | 1.00 | 44.94 | C |
| ATOM | 30 | O | SER | A | 16 | 36.948 | −79.885 | −19.632 | 1.00 | 48.25 | O |
| ATOM | 31 | N | GLY | A | 17 | 35.514 | −80.404 | −21.273 | 1.00 | 43.14 | N |
| ATOM | 32 | CA | GLY | A | 17 | 35.422 | −81.810 | −20.875 | 1.00 | 37.29 | C |
| ATOM | 33 | C | GLY | A | 17 | 35.626 | −82.689 | −22.080 | 1.00 | 38.75 | C |
| ATOM | 34 | O | GLY | A | 17 | 35.524 | −82.223 | −23.714 | 1.00 | 40.58 | O |
| ATOM | 35 | N | ARG | A | 18 | 35.947 | −83.958 | −21.343 | 1.00 | 41.31 | N |
| ATOM | 36 | CA | ARG | A | 18 | 36.067 | −84.944 | −22.918 | 1.00 | 42.34 | C |
| ATOM | 37 | CB | ARG | A | 18 | 37.282 | −85.847 | −22.674 | 1.00 | 45.27 | C |
| ATOM | 38 | CG | ARG | A | 18 | 37.482 | −86.949 | −23.714 | 1.00 | 44.17 | C |
| ATOM | 39 | CD | ARG | A | 18 | 38.144 | −86.403 | −24.971 | 1.00 | 47.47 | C |
| ATOM | 40 | NE | ARG | A | 18 | 38.462 | −87.492 | −25.885 | 1.00 | 54.42 | N |
| ATOM | 41 | CZ | ARG | A | 18 | 37.834 | −87.727 | −27.032 | 1.00 | 52.83 | C |
| ATOM | 42 | NH1 | ARG | A | 18 | 36.845 | −86.943 | −27.441 | 1.00 | 52.72 | N |
| ATOM | 43 | NH2 | ARG | A | 18 | 38.199 | −88.762 | −27.770 | 1.00 | 54.41 | N |
| ATOM | 44 | C | ARG | A | 18 | 34.811 | −85.808 | −22.989 | 1.00 | 42.63 | C |
| ATOM | 45 | O | ARG | A | 18 | 34.305 | −86.278 | −21.967 | 1.00 | 46.05 | O |
| ATOM | 46 | N | ASP | A | 19 | 34.307 | −86.035 | −24.191 | 1.00 | 44.38 | N |
| ATOM | 47 | CA | ASP | A | 19 | 33.152 | −86.904 | −24.323 | 1.00 | 43.07 | C |
| ATOM | 48 | CB | ASP | A | 19 | 32.088 | −86.304 | −25.254 | 1.00 | 42.92 | C |
| ATOM | 49 | CG | ASP | A | 19 | 30.951 | −87.295 | −25.542 | 1.00 | 39.47 | C |
| ATOM | 50 | CD1 | ASP | A | 19 | 30.436 | −87.883 | −24.571 | 1.00 | 38.24 | O |
| ATOM | 51 | CD2 | ASP | A | 19 | 30.624 | −87.523 | −26.729 | 1.00 | 38.71 | O |
| ATOM | 52 | C | ASP | A | 19 | 33.553 | −88.269 | −24.831 | 1.00 | 41.15 | C |
| ATOM | 53 | O | ASP | A | 19 | 33.970 | −88.407 | −25.965 | 1.00 | 46.41 | O |
| ATOM | 54 | N | LEU | A | 20 | 33.391 | −89.288 | −24.005 | 1.00 | 42.15 | N |
| ATOM | 55 | CA | LEU | A | 20 | 33.737 | −90.634 | −24.430 | 1.00 | 44.12 | C |
| ATOM | 56 | CB | LEU | A | 20 | 34.843 | −91.221 | −23.550 | 1.00 | 45.75 | C |
| ATOM | 57 | CG | LEU | A | 20 | 36.167 | −90.485 | −23.656 | 1.00 | 51.98 | C |
| ATOM | 58 | CD1 | LEU | A | 20 | 37.184 | −91.164 | −22.760 | 1.00 | 56.66 | C |
| ATOM | 59 | CD2 | LEU | A | 20 | 36.640 | −90.447 | −25.104 | 1.00 | 56.56 | C |
| ATOM | 60 | C | LEU | A | 20 | 32.541 | −91.541 | −24.378 | 1.00 | 44.35 | C |
| ATOM | 61 | O | LEU | A | 20 | 31.721 | −91.450 | −23.463 | 1.00 | 44.85 | O |
| ATOM | 62 | N | ASN | A | 21 | 32.458 | −92.453 | −25.332 | 1.00 | 43.05 | N |
| ATOM | 63 | CA | ASN | A | 21 | 31.398 | −93.418 | −25.270 | 1.00 | 44.96 | C |
| ATOM | 64 | CB | ASN | A | 21 | 30.444 | −93.280 | −26.457 | 1.00 | 47.44 | C |
| ATOM | 65 | CG | ASN | A | 21 | 29.042 | −93.773 | −26.129 | 1.00 | 48.74 | C |
| ATOM | 66 | CD1 | ASN | A | 21 | 28.500 | −93.487 | −25.054 | 1.00 | 47.63 | O |
| ATOM | 67 | ND2 | ASN | A | 21 | 28.454 | −94.537 | −27.049 | 1.00 | 47.32 | N |
| ATOM | 68 | C | ASN | A | 21 | 31.940 | −94.826 | −25.110 | 1.00 | 47.57 | C |
| ATOM | 69 | O | ASN | A | 21 | 31.220 | −95.795 | −25.401 | 1.00 | 42.52 | O |
| ATOM | 70 | N | CYS | A | 22 | 33.198 | −94.906 | −24.628 | 1.00 | 45.93 | N |
| ATOM | 71 | CA | CYS | A | 22 | 33.921 | −96.160 | −24.323 | 1.00 | 46.67 | C |
| ATOM | 72 | CB | CYS | A | 22 | 34.687 | −96.659 | −25.561 | 1.00 | 53.16 | C |
| ATOM | 73 | SG | CYS | A | 22 | 35.916 | −97.979 | −25.258 | 1.00 | 47.99 | S |
| ATOM | 74 | C | CYS | A | 22 | 34.920 | −95.994 | −23.159 | 1.00 | 46.07 | C |
| ATOM | 75 | O | CYS | A | 22 | 35.904 | −95.247 | −23.282 | 1.00 | 43.39 | O |
| ATOM | 76 | N | VAL | A | 23 | 31.677 | −96.689 | −22.044 | 1.00 | 46.76 | N |
| ATOM | 77 | CA | VAL | A | 23 | 35.536 | −96.566 | −20.856 | 1.00 | 51.47 | C |
| ATOM | 78 | CB | VAL | A | 23 | 34.926 | −95.667 | −19.761 | 1.00 | 54.45 | C |
| ATOM | 79 | CG1 | VAL | A | 23 | 35.880 | −95.578 | −18.568 | 1.00 | 55.35 | C |
| ATOM | 80 | CG2 | VAL | A | 23 | 34.635 | −94.280 | −20.299 | 1.00 | 54.00 | C |
| ATOM | 81 | C | VAL | A | 23 | 35.838 | −97.923 | −20.240 | 1.00 | 51.91 | C |
| ATOM | 82 | O | VAL | A | 23 | 35.068 | −98.403 | −19.398 | 1.00 | 54.29 | O |
| ATOM | 83 | N | PRO | A | 24 | 36.956 | −98.553 | −20.666 | 1.00 | 53.39 | N |
| ATOM | 84 | CA | PRO | A | 24 | 37.309 | −99.901 | −20.191 | 1.00 | 50.15 | C |
| ATOM | 85 | CB | PRO | A | 24 | 38.543 | −100.256 | −21.037 | 1.00 | 50.52 | C |
| ATOM | 86 | CG | PRO | A | 24 | 38.395 | −99.412 | −22.280 | 1.00 | 49.94 | C |
| ATOM | 87 | CD | PRO | A | 24 | 37.852 | −98.112 | −21.758 | 1.00 | 50.44 | C |
| ATOM | 88 | C | PRO | A | 24 | 37.612 | −99.949 | −18.690 | 1.00 | 49.61 | C |
| ATOM | 89 | O | PRO | A | 24 | 37.123 | −100.837 | −17.982 | 1.00 | 47.73 | O |
| ATOM | 80 | N | GLU | A | 25 | 38.395 | −98.995 | −18.203 | 1.00 | 49.73 | N |
| ATOM | 81 | CA | GLU | A | 25 | 36.641 | −98.906 | −16.778 | 1.00 | 52.26 | C |
| ATOM | 82 | CB | GLU | A | 25 | 39.972 | −99.567 | −16.410 | 1.00 | 63.06 | C |
| ATOM | 83 | CG | GLU | A | 25 | 40.024 | −100.117 | −14.991 | 1.00 | 76.38 | C |
| ATOM | 84 | CD | GLU | A | 25 | 40.888 | −101.368 | −14.891 | 1.00 | 88.79 | C |
| ATOM | 85 | OE1 | GLU | A | 25 | 42.117 | −101.239 | −14.669 | 1.00 | 85.79 | O |
| ATOM | 86 | OE2 | GLU | A | 25 | 40.330 | −102.485 | −15.037 | 1.00 | 93.22 | O |
| ATOM | 87 | C | GLU | A | 25 | 30.622 | −97.448 | −16.377 | 1.00 | 50.60 | C |
| ATOM | 88 | O | GLU | A | 25 | 39.435 | −96.644 | −16.853 | 1.00 | 47.08 | O |
| ATOM | 89 | N | ILE | A | 26 | 37.687 | −97.126 | −15.490 | 1.00 | 48.75 | N |
| ATOM | 100 | CA | ILE | A | 26 | 37.386 | −95.742 | −15.098 | 1.00 | 50.56 | C |
| ATOM | 101 | C8 | ILE | A | 26 | 36.137 | −95.694 | −14.173 | 1.00 | 51.46 | C |
| ATOM | 102 | CG1 | ILE | A | 26 | 35.619 | −94.275 | −14.007 | 1.00 | 50.20 | C |
| ATOM | 103 | CD1 | ILE | A | 26 | 34.189 | −94.242 | −13.516 | 1.00 | 55.64 | C |
| ATOM | 104 | CG2 | ILE | A | 26 | 36.439 | −96.298 | −12.811 | 1.00 | 58.67 | C |
| ATOM | 105 | C | ILE | A | 26 | 38.575 | −94.960 | −14.506 | 1.00 | 47.79 | C |
| ATOM | 106 | O | ILE | A | 26 | 38.845 | −93.828 | −14.916 | 1.00 | 49.26 | O |
| ATOM | 107 | N | ALA | A | 27 | 39.266 | −95.571 | −13.548 | 1.00 | 51.56 | N |
| ATOM | 108 | CA | ALA | A | 27 | 40.511 | −95.041 | −12.973 | 1.00 | 51.31 | C |
| ATOM | 109 | CB | ALA | A | 27 | 41.209 | −96.124 | −12.157 | 1.00 | 52.36 | C |
| ATOM | 110 | C | ALA | A | 27 | 41.454 | −94.549 | −14.047 | 1.00 | 49.09 | C |
| ATOM | 111 | O | ALA | A | 27 | 41.858 | −93.395 | −14.033 | 1.00 | 46.70 | O |
| ATOM | 112 | N | ASP | A | 28 | 41.792 | −95.444 | −14.974 | 1.00 | 51.57 | N |
| ATOM | 113 | CA | ASP | A | 28 | 42.753 | −95.162 | −16.033 | 1.00 | 54.74 | C |
| ATOM | 114 | CB | ASP | A | 28 | 43.062 | −96.417 | −16.845 | 1.00 | 62.89 | C |
| ATOM | 115 | CG | ASP | A | 28 | 43.928 | −97.456 | −16.064 | 1.00 | 72.12 | C |
| ATOM | 116 | OD1 | ASP | A | 28 | 44.749 | −97.027 | −15.212 | 1.00 | 71.84 | O |
| ATOM | 117 | OD2 | ASP | A | 28 | 43.794 | −98.683 | −16.314 | 1.00 | 66.96 | O |
| ATOM | 118 | C | ASP | A | 28 | 42.286 | −94.035 | −16.947 | 1.00 | 55.22 | C |
| ATOM | 119 | O | ASP | A | 28 | 43.008 | −93.043 | −17.156 | 1.00 | 53.26 | O |
| ATOM | 120 | N | THR | A | 29 | 41.067 | −94.182 | −17.469 | 1.00 | 56.49 | N |
| ATOM | 121 | CA | THR | A | 29 | 40.500 | −93.217 | −18.415 | 1.00 | 55.16 | C |
| ATOM | 122 | CB | THR | A | 29 | 39.079 | −93.618 | −18.847 | 1.00 | 56.25 | C |
| ATOM | 123 | OG1 | THR | A | 29 | 39.087 | −94.958 | −19.353 | 1.00 | 55.24 | O |
| ATOM | 124 | CG2 | THR | A | 29 | 38.560 | −92.679 | −19.938 | 1.00 | 58.15 | C |
| ATOM | 125 | C | THR | A | 29 | 40.500 | −91.797 | −17.844 | 1.00 | 55.34 | C |
| ATOM | 126 | O | THR | A | 29 | 40.972 | −90.855 | −18.490 | 1.00 | 53.61 | O |
| ATOM | 127 | N | LEU | A | 30 | 39.985 | −91.663 | −16.624 | 1.00 | 55.91 | N |
| ATOM | 128 | CA | LEU | A | 30 | 39.960 | −90.391 | −15.928 | 1.00 | 53.25 | C |
| ATOM | 129 | CB | LEU | A | 30 | 39.294 | −90.548 | −14.561 | 1.00 | 51.97 | C |
| ATOM | 130 | CG | LEU | A | 30 | 38.833 | −89.248 | −13.899 | 1.00 | 54.50 | C |
| ATOM | 131 | CD1 | LEU | A | 30 | 38.095 | −88.345 | −14.890 | 1.00 | 51.69 | C |
| ATOM | 132 | CD2 | LEU | A | 30 | 37.997 | −89.530 | −12.649 | 1.00 | 52.12 | C |
| ATOM | 133 | C | LEU | A | 30 | 41.359 | −89.799 | −15.790 | 1.00 | 53.75 | C |
| ATOM | 134 | O | LEU | A | 30 | 41.550 | −88.602 | −15.996 | 1.00 | 56.18 | O |
| ATOM | 135 | N | GLY | A | 31 | 42.340 | −90.635 | −15.459 | 1.00 | 56.22 | N |
| ATOM | 136 | CA | GLY | A | 31 | 43.730 | −90.185 | −15.414 | 1.00 | 52.35 | C |
| ATOM | 137 | C | GLY | A | 31 | 44.191 | −89.685 | −16.772 | 1.00 | 53.05 | C |
| ATOM | 138 | O | GLY | A | 31 | 44.752 | −88.593 | −16.884 | 1.00 | 58.21 | O |
| ATOM | 139 | N | ALA | A | 32 | 43.941 | −90.476 | −17.812 | 1.00 | 52.01 | N |
| ATOM | 140 | CA | ALA | A | 32 | 44.362 | −90.108 | −19.160 | 1.00 | 55.50 | C |
| ATOM | 141 | CB | ALA | A | 32 | 43.901 | −91.146 | −20.171 | 1.00 | 55.48 | C |
| ATOM | 142 | C | ALA | A | 32 | 43.862 | −88.720 | −19.544 | 1.00 | 59.96 | C |
| ATOM | 143 | O | ALA | A | 32 | 44.660 | −87.857 | −19.962 | 1.00 | 62.06 | O |
| ATOM | 144 | N | VAL | A | 33 | 42.554 | −88.499 | −19.370 | 1.00 | 58.96 | N |
| ATOM | 145 | CA | VAL | A | 33 | 41.927 | −87.237 | −19.792 | 1.00 | 56.97 | C |
| ATOM | 146 | CB | VAL | A | 33 | 40.365 | −87.294 | −19.918 | 1.00 | 53.93 | C |
| ATOM | 147 | CG1 | VAL | A | 33 | 39.889 | −88.588 | −20.582 | 1.00 | 50.72 | C |
| ATOM | 148 | CG2 | VAL | A | 33 | 39.674 | −87.005 | −18.587 | 1.00 | 48.46 | C |
| ATOM | 149 | C | VAL | A | 33 | 42.348 | −86.069 | −18.914 | 1.00 | 56.79 | C |
| ATOM | 150 | C | VAL | A | 33 | 42.431 | −84.938 | −19.398 | 1.00 | 63.24 | O |
| ATOM | 151 | N | ALA | A | 34 | 42.614 | −81.335 | −17.636 | 1.00 | 53.84 | N |
| ATOM | 152 | CA | ALA | A | 34 | 43.066 | −85.283 | −16.735 | 1.00 | 56.78 | C |
| ATOM | 153 | CB | ALA | A | 34 | 43.033 | −85.759 | −15.296 | 1.00 | 57.34 | C |
| ATOM | 154 | C | ALA | A | 34 | 44.470 | −84.826 | −17.139 | 1.00 | 62.40 | C |
| ATOM | 155 | C | ALA | A | 34 | 44.711 | −83.627 | −17.306 | 1.00 | 63.87 | O |
| ATOM | 156 | N | LYS | A | 35 | 45.374 | −85.791 | −17.326 | 1.00 | 67.37 | N |
| ATOM | 157 | CA | LYS | A | 35 | 46.716 | −85.530 | −17.856 | 1.00 | 73.82 | C |
| ATOM | 158 | CB | LYS | A | 35 | 47.504 | −86.854 | −18.044 | 1.00 | 78.84 | C |
| ATOM | 159 | CG | LYS | A | 35 | 48.303 | −87.021 | −19.342 | 1.00 | 85.82 | C |
| ATOM | 160 | CD | LYS | A | 35 | 49.667 | −86.331 | −19.315 | 1.00 | 88.65 | C |
| ATOM | 161 | CE | LYS | A | 35 | 50.324 | −86.339 | −20.693 | 1.00 | 86.24 | C |
| ATOM | 162 | NZ | LYS | A | 35 | 51.261 | −85.191 | −20.877 | 1.00 | 77.49 | N |
| ATOM | 163 | C | LYS | A | 35 | 46.618 | −84.711 | −19.141 | 1.00 | 69.94 | C |
| ATOM | 164 | O | LYS | A | 35 | 47.367 | −83.752 | −19.325 | 1.00 | 67.05 | O |
| ATOM | 165 | N | GLN | A | 36 | 45.667 | −85.074 | −20.001 | 1.00 | 67.46 | N |
| ATOM | 166 | CA | GLN | A | 36 | 45.458 | −84.372 | −21.274 | 1.00 | 63.74 | C |
| ATOM | 167 | CB | GLN | A | 36 | 44.483 | −85.139 | −22.163 | 1.00 | 65.83 | C |
| ATOM | 168 | CG | GLN | A | 36 | 45.081 | −86.370 | −22.832 | 1.00 | 67.27 | C |
| ATOM | 169 | CD | GLN | A | 36 | 44.030 | −87.263 | −23.495 | 1.00 | 77.35 | C |
| ATOM | 170 | 0E1 | GLN | A | 36 | 42.912 | −86.826 | −23.806 | 1.00 | 73.26 | O |
| ATOM | 171 | NE2 | GLN | A | 36 | 44.390 | −88.526 | −23.717 | 1.00 | 76.70 | N |
| ATOM | 172 | C | GLN | A | 36 | 45.019 | −82.912 | −21.132 | 1.00 | 62.16 | C |
| ATOM | 173 | O | GLN | A | 36 | 45.178 | −82.136 | −22.068 | 1.00 | 66.17 | O |
| ATOM | 174 | N | GLY | A | 37 | 44.482 | −82.533 | −19.972 | 1.00 | 62.21 | N |
| ATOM | 175 | CA | GLY | A | 37 | 44.135 | −81.126 | −19.707 | 1.00 | 58.15 | C |
| ATOM | 176 | C | GLY | A | 37 | 42.675 | −80.847 | −19.371 | 1.00 | 57.47 | C |
| ATOM | 177 | O | GLY | A | 37 | 42.357 | −79.785 | −18.835 | 1.00 | 55.08 | O |
| ATOM | 178 | N | PHE | A | 38 | 41.800 | −81.812 | −19.670 | 1.00 | 53.21 | N |
| ATOM | 179 | CA | PHE | A | 38 | 40.354 | −81.718 | −19.442 | 1.00 | 51.67 | C |
| ATOM | 180 | CB | PHE | A | 38 | 39.661 | −82.916 | −20.038 | 1.00 | 50.27 | C |
| ATOM | 181 | CG | PHE | A | 38 | 39.703 | −83.013 | −21.544 | 1.00 | 48.49 | C |
| ATOM | 182 | CD1 | PHE | A | 38 | 38.892 | −82.170 | −22.331 | 1.00 | 46.35 | C |
| ATOM | 183 | CE1 | PHE | A | 38 | 38.929 | −82.216 | −23.700 | 1.00 | 46.39 | C |
| ATOM | 184 | C1 | PHE | A | 38 | 39.782 | −83.112 | −24.341 | 1.00 | 49.64 | C |
| ATOM | 185 | CE2 | PHE | A | 38 | 40.594 | −83.965 | −21.592 | 1.00 | 48.14 | C |
| ATOM | 186 | CD2 | PHE | A | 38 | 40.551 | −83.910 | −22.202 | 1.00 | 49.31 | C |
| ATOM | 187 | C | PHE | A | 38 | 39.888 | −81.528 | −17.985 | 1.00 | 53.90 | C |
| ATOM | 188 | O | PHE | A | 38 | 40.386 | −82.197 | −17.068 | 1.00 | 54.54 | O |
| ATOM | 189 | N | ASP | A | 39 | 38.901 | −80.645 | −17.789 | 1.00 | 54.37 | N |
| ATOM | 190 | CA | ASP | A | 39 | 38.264 | −80.432 | −16.467 | 1.00 | 51.80 | C |
| ATOM | 191 | CB | ASP | A | 39 | 37.487 | −79.121 | −16.425 | 1.00 | 57.11 | C |
| ATOM | 192 | CG | ASP | A | 39 | 38.368 | −77.927 | −16.341 | 1.00 | 61.82 | C |
| ATOM | 193 | OD1 | ASP | A | 39 | 39.368 | −78.040 | −16.649 | 1.00 | 68.92 | O |
| ATOM | 194 | OD2 | ASP | A | 39 | 37.850 | −76.857 | −15.973 | 1.00 | 75.96 | O |
| ATOM | 195 | C | ASP | A | 39 | 37.312 | −81.534 | −16.043 | 1.00 | 43.15 | C |
| ATOM | 196 | O | ASP | A | 39 | 37.101 | −81.748 | −14.854 | 1.00 | 41.16 | O |
| ATOM | 197 | N | PHE | A | 40 | 36.731 | −82.228 | −17.010 | 1.00 | 41.65 | N |
| ATOM | 198 | CA | PHE | A | 40 | 35.733 | −83.265 | −16.705 | 1.00 | 42.72 | C |
| ATOM | 199 | CB | PHE | A | 40 | 34.381 | −82.642 | −16.271 | 1.00 | 41.97 | C |
| ATOM | 200 | CG | PHE | A | 40 | 33.673 | −81.880 | −17.361 | 1.00 | 40.15 | C |
| ATOM | 201 | CD1 | PHE | A | 40 | 33.988 | −80.550 | −17.620 | 1.00 | 40.52 | C |
| ATOM | 202 | CE1 | PHE | A | 40 | 33.329 | −79.845 | −18.637 | 1.00 | 43.10 | C |
| ATOM | 203 | CZ | PHE | A | 40 | 32.337 | −80.472 | −19.396 | 1.00 | 39.60 | C |
| ATOM | 204 | CE2 | PHE | A | 40 | 32.009 | −81.802 | −19.145 | 1.00 | 39.17 | C |
| ATOM | 205 | CD2 | PHE | A | 40 | 32.674 | −82.499 | −18.132 | 1.00 | 42.51 | C |
| ATOM | 206 | C | PHE | A | 40 | 35.517 | −84.222 | −17.866 | 1.00 | 41.30 | C |
| ATOM | 207 | O | PHE | A | 40 | 35.869 | −83.929 | −19.010 | 1.00 | 41.24 | O |
| ATOM | 208 | N | LEU | A | 41 | 34.902 | −85.347 | −17.548 | 1.00 | 41.82 | N |
| ATOM | 209 | CA | LEU | A | 41 | 34.667 | −86.429 | −18.476 | 1.00 | 45.56 | C |
| ATOM | 210 | CB | LEU | A | 41 | 35.328 | −87.708 | −17.925 | 1.00 | 44.26 | C |
| ATOM | 211 | CG | LEU | A | 41 | 35.120 | −88.971 | −18.767 | 1.00 | 44.01 | C |
| ATOM | 212 | CD1 | LEU | A | 41 | 35.645 | −88.779 | −20.188 | 1.00 | 41.49 | C |
| ATOM | 213 | CD2 | LEU | A | 41 | 35.755 | −90.192 | −18.122 | 1.00 | 43.26 | C |
| ATOM | 214 | C | LEU | A | 41 | 33.180 | −86.699 | −18.620 | 1.00 | 46.17 | C |
| ATOM | 215 | O | LEU | A | 41 | 32.469 | −86.747 | −17.604 | 1.00 | 45.56 | O |
| ATOM | 216 | N | CYS | A | 42 | 32.713 | −86.893 | −19.861 | 1.00 | 43.71 | N |
| ATOM | 217 | CA | CYS | A | 42 | 31.341 | −87.375 | −20.102 | 1.00 | 43.71 | C |
| ATOM | 218 | CB | CYS | A | 42 | 30.605 | −86.536 | −21.141 | 1.00 | 40.61 | C |
| ATOM | 219 | SG | CYS | A | 42 | 30.860 | −84.769 | −21.005 | 1.00 | 41.12 | S |
| ATOM | 220 | C | CYS | A | 42 | 31.338 | −88.827 | −20.550 | 1.00 | 45.55 | C |
| ATOM | 221 | O | CYS | A | 42 | 31.890 | −89.170 | −21.590 | 1.00 | 49.51 | O |
| ATOM | 222 | N | MET | A | 43 | 30.678 | −89.676 | −19.781 | 1.00 | 47.78 | N |
| ATOM | 223 | CA | MET | A | 43 | 30.769 | −91.102 | −20.001 | 1.00 | 44.97 | C |
| ATOM | 224 | CB | MET | A | 43 | 31.957 | −91.665 | −19.237 | 1.00 | 49.85 | C |
| ATOM | 225 | CG | MET | A | 43 | 32.133 | −91.098 | −17.845 | 1.00 | 50.50 | C |
| ATOM | 226 | SD | MET | A | 43 | 33.153 | −92.182 | −16.826 | 1.00 | 53.81 | S |
| ATOM | 227 | CE | MET | A | 43 | 31.902 | −93.270 | −16.131 | 1.00 | 47.86 | C |
| ATOM | 228 | C | MET | A | 43 | 29.512 | −91.830 | −19.573 | 1.00 | 45.32 | C |
| ATOM | 229 | O | MET | A | 43 | 28.711 | −91.303 | −18.813 | 1.00 | 44.80 | O |
| ATOM | 230 | N | PRO | A | 44 | 29.337 | −93.051 | −20.077 | 1.00 | 43.19 | N |
| ATOM | 231 | CA | PRO | A | 44 | 28.222 | −93.908 | −19.719 | 1.00 | 44.54 | C |
| ATOM | 232 | CB | PRO | A | 44 | 28.460 | −95.148 | −20.581 | 1.00 | 43.57 | C |
| ATOM | 233 | CG | PRO | A | 44 | 29.166 | −94.606 | −21.775 | 1.00 | 42.01 | C |
| ATOM | 234 | CD | PRO | A | 44 | 30.094 | −93.580 | −21.225 | 1.00 | 41.10 | C |
| ATOM | 235 | C | PRO | A | 44 | 28.277 | −94.293 | −18.255 | 1.00 | 44.82 | C |
| ATOM | 236 | O | PRO | A | 44 | 29.366 | −94.437 | −17.711 | 1.00 | 44.07 | O |
| ATOM | 237 | N | VAL | A | 45 | 27.116 | −94.471 | −17.630 | 1.00 | 40.28 | N |
| ATOM | 238 | CA | VAL | A | 45 | 27.090 | −94.985 | −16.235 | 1.00 | 40.48 | C |
| ATOM | 239 | CB | VAL | A | 45 | 26.128 | −94.157 | −15.323 | 1.00 | 42.97 | C |
| ATOM | 240 | CG1 | VAL | A | 45 | 26.786 | −92.366 | −14.857 | 1.00 | 43.68 | C |
| ATOM | 241 | CG2 | VAL | A | 45 | 24.785 | −93.370 | −15.985 | 1.00 | 44.34 | C |
| ATOM | 242 | C | VAL | A | 45 | 26.775 | −96.460 | −16.241 | 1.00 | 38.58 | C |
| ATOM | 243 | O | VAL | A | 45 | 26.968 | −97.149 | −15.241 | 1.00 | 40.05 | O |
| ATOM | 244 | N | PHE | A | 46 | 26.287 | −96.935 | −17.375 | 1.00 | 36.96 | N |
| ATOM | 245 | CA | PHE | A | 46 | 26.105 | −98.340 | −17.666 | 1.00 | 39.74 | C |
| ATOM | 246 | CB | PHE | A | 46 | 24.609 | −98.582 | −17.774 | 1.00 | 38.39 | C |
| ATOM | 247 | CG | PHE | A | 46 | 23.906 | −98.719 | −16.446 | 1.00 | 37.43 | C |
| ATOM | 248 | CD1 | PHE | A | 46 | 23.769 | −97.563 | −15.678 | 1.00 | 37.41 | C |
| ATOM | 249 | CE1 | PHE | A | 46 | 23.137 | −97.603 | −14.445 | 1.00 | 36.66 | C |
| ATOM | 250 | CZ | PHE | A | 46 | 22.613 | −98.793 | −13.968 | 1.00 | 37.35 | C |
| ATOM | 251 | CE2 | PHE | A | 46 | 22.733 | −99.948 | −14.727 | 1.00 | 41.05 | C |
| ATOM | 252 | CD2 | PHE | A | 46 | 23.387 | −99.909 | −15.954 | 1.00 | 38.99 | C |
| ATOM | 253 | C | PHE | A | 46 | 26.790 | −98.536 | −19.002 | 1.00 | 42.15 | C |
| ATOM | 254 | O | PHE | A | 46 | 26.979 | −97.559 | −19.735 | 1.00 | 45.84 | O |
| ATOM | 255 | N | HIS | A | 47 | 27.184 | −99.770 | −19.324 | 1.00 | 42.26 | N |
| ATOM | 256 | CA | HIS | A | 47 | 27.818 | −100.027 | −20.621 | 1.00 | 42.18 | C |
| ATOM | 257 | CB | HIS | A | 47 | 28.317 | −101.464 | −20.735 | 1.00 | 43.74 | C |
| ATOM | 258 | CG | HIS | A | 47 | 29.181 | −101.705 | −21.937 | 1.00 | 47.97 | C |
| ATOM | 259 | ND1 | HIS | A | 47 | 28.672 | −101.798 | −23.215 | 1.00 | 51.07 | N |
| ATOM | 260 | CE1 | HIS | A | 47 | 29.659 | −102.012 | −24.067 | 1.00 | 48.12 | C |
| ATOM | 261 | NE2 | HIS | A | 47 | 30.786 | −102.082 | −23.385 | 1.00 | 44.51 | N |
| ATOM | 262 | CD2 | HIS | A | 47 | 30.517 | −101.883 | −22.053 | 1.00 | 42.27 | C |
| ATOM | 263 | C | HIS | A | 47 | 26.842 | −99.724 | −21.764 | 1.00 | 39.75 | C |
| ATOM | 264 | O | HIS | A | 47 | 25.714 | −100.228 | −21.776 | 1.00 | 40.75 | O |
| ATOM | 265 | N | PRO | A | 48 | 27.275 | −98.916 | −22.731 | 1.00 | 35.84 | N |
| ATOM | 266 | CA | PRO | A | 48 | 26.324 | −98.449 | −23.738 | 1.00 | 37.78 | C |
| ATOM | 267 | CB | PRO | A | 48 | 27.182 | −97.615 | −24.685 | 1.00 | 36.17 | C |
| ATOM | 268 | CG | PRO | A | 48 | 28.285 | −97.120 | −23.825 | 1.00 | 36.82 | C |
| ATOM | 269 | CD | PRO | A | 48 | 28.524 | −96.141 | −22.742 | 1.00 | 35.01 | C |
| ATOM | 270 | C | PRO | A | 48 | 25.630 | −99.546 | −24.507 | 1.00 | 40.55 | C |
| ATOM | 271 | O | PRO | A | 48 | 24.569 | −99.288 | −25.080 | 1.00 | 37.73 | O |
| ATOM | 272 | N | ARG | A | 49 | 26.219 | −100.747 | −24.536 | 1.00 | 38.73 | N |
| ATOM | 273 | CA | ARG | A | 49 | 25.627 | −101.856 | −25.306 | 1.00 | 38.35 | C |
| ATOM | 274 | CB | ARG | A | 49 | 26.690 | −102.658 | −26.073 | 1.00 | 36.44 | C |
| ATOM | 275 | CG | ARG | A | 49 | 27.152 | −101.949 | −27.329 | 1.00 | 40.94 | C |
| ATOM | 276 | CD | ARG | A | 49 | 28.070 | −102.835 | −28.149 | 1.00 | 44.41 | C |
| ATOM | 277 | NE | ARG | A | 49 | 29.394 | −103.033 | −27.538 | 1.00 | 39.01 | N |
| ATOM | 278 | CZ | ARG | A | 49 | 29.868 | −104.209 | −27.116 | 1.00 | 39.18 | C |
| ATOM | 279 | NH1 | ARG | A | 49 | 29.127 | −105.321 | −27.190 | 1.00 | 33.87 | N |
| ATOM | 280 | NH2 | ARG | A | 49 | 31.093 | −104.273 | −26.593 | 1.00 | 36.04 | N |
| ATOM | 281 | C | ARG | A | 49 | 24.852 | −102.787 | −24.426 | 1.00 | 35.38 | C |
| ATOM | 282 | C | ARG | A | 49 | 24.274 | −103.771 | −24.905 | 1.00 | 34.53 | O |
| ATOM | 283 | N | PHE | A | 50 | 24.853 | −102.485 | −23.133 | 1.00 | 33.74 | N |
| ATOM | 784 | CA | PHE | A | 50 | 24.317 | −103.400 | −22.149 | 1.00 | 35.24 | C |
| ATOM | 285 | CB | PHE | A | 50 | 25.033 | −103.214 | −20.799 | 1.00 | 33.93 | C |
| ATOM | 286 | CG | PHE | A | 50 | 24.754 | −104.316 | −19.810 | 1.00 | 38.08 | C |
| ATOM | 287 | CD1 | PHE | A | 50 | 24.516 | −105.622 | −20.246 | 1.00 | 42.56 | C |
| ATOM | 288 | CE1 | PHE | A | 50 | 24.254 | −106.648 | −19.343 | 1.00 | 44.24 | C |
| ATOM | 289 | CZ | PHE | A | 50 | 24.228 | −106.374 | −17.985 | 1.00 | 45.41 | C |
| ATOM | 290 | CE2 | PHE | A | 50 | 24.463 | −105.081 | −17.539 | 1.00 | 41.34 | C |
| ATOM | 291 | CD2 | PHE | A | 50 | 24.725 | −104.060 | −18.451 | 1.00 | 39.31 | C |
| ATOM | 292 | C | PHE | A | 50 | 22.773 | −103.307 | −22.027 | 1.00 | 34.60 | C |
| ATOM | 293 | C | PHE | A | 50 | 22.244 | −103.045 | −20.960 | 1.00 | 37.13 | O |
| ATOM | 294 | N | LYS | A | 51 | 22.077 | −103.541 | −23.129 | 1.00 | 45.06 | N |
| ATOM | 295 | CA | LYS | A | 51 | 20.612 | −103.528 | −23.175 | 1.00 | 41.27 | C |
| ATOM | 296 | CB | LYS | A | 51 | 20.187 | −104.084 | −24.522 | 1.00 | 45.24 | C |
| ATOM | 297 | CG | LYS | A | 51 | 19.133 | −103.291 | −25.262 | 1.00 | 51.67 | C |
| ATOM | 298 | CD | LYS | A | 51 | 19.388 | −103.388 | −26.760 | 1.00 | 54.12 | C |
| ATOM | 299 | CE | LYS | A | 51 | 18.069 | −103.394 | −27.522 | 1.00 | 58.53 | C |
| ATOM | 300 | NZ | LYS | A | 51 | 18.290 | −103.493 | −28.991 | 1.00 | 63.15 | N |
| ATOM | 301 | C | LYS | A | 51 | 20.024 | −104.393 | −22.073 | 1.00 | 41.98 | C |
| ATOM | 302 | O | LYS | A | 51 | 20.302 | −105.576 | −22.039 | 1.00 | 45.08 | O |
| ATOM | 303 | N | ARG | A | 52 | 19.246 | −103.803 | −21.159 | 1.00 | 46.47 | N |
| ATOM | 304 | CA | ARG | A | 52 | 18.569 | −104.536 | −20.064 | 1.00 | 44.80 | C |
| ATOM | 305 | CB | ARG | A | 52 | 18.643 | −103.763 | −18.752 | 1.00 | 43.33 | C |
| ATOM | 306 | CG | ARG | A | 52 | 19.649 | −104.038 | −17.895 | 1.00 | 47.51 | C |
| ATOM | 307 | CD | ARG | A | 52 | 20.929 | −103.002 | −18.118 | 1.00 | 48.49 | C |
| ATOM | 308 | NE | ARG | A | 52 | 20.537 | −101.659 | −17.685 | 1.00 | 42.10 | N |
| ATOM | 309 | CZ | ARG | A | 52 | 21.089 | −100.551 | −13.158 | 1.00 | 41.16 | C |
| ATOM | 310 | NH1 | ARG | A | 52 | 22.056 | −100.615 | −19.071 | 1.00 | 44.69 | N |
| ATOM | 311 | NH2 | ARG | A | 52 | 20.689 | −99.376 | −17.724 | 1.00 | 43.08 | N |
| ATOM | 312 | C | ARG | A | 52 | 17.097 | −104.787 | −20.350 | 1.00 | 43.82 | C |
| ATOM | 313 | O | ARG | A | 52 | 16.489 | −104.089 | −21.144 | 1.00 | 47.99 | O |
| ATOM | 314 | N | GLU | A | 53 | 16.521 | −105.747 | −19.633 | 1.00 | 44.99 | N |
| ATOM | 315 | CA | GLU | A | 53 | 15.147 | −106.171 | −19.818 | 1.00 | 42.21 | C |
| ATOM | 316 | CB | GLU | A | 53 | 15.121 | −107.554 | −20.471 | 1.00 | 39.84 | C |
| ATOM | 317 | CG | GLU | A | 53 | 13.806 | −108.323 | −20.352 | 1.00 | 41.49 | C |
| ATOM | 318 | CD | GLU | A | 53 | 12.636 | −107.654 | −21.076 | 1.00 | 43.47 | C |
| ATOM | 319 | CE1 | GLU | A | 53 | 12.834 | −107.114 | −22.198 | 1.00 | 40.20 | O |
| ATOM | 320 | CE2 | GLU | A | 53 | 11.508 | −107.673 | −20.523 | 1.00 | 47.10 | O |
| ATOM | 321 | C | GLU | A | 53 | 14.464 | −106.189 | −18.445 | 1.00 | 46.48 | C |
| ATOM | 322 | O | GLU | A | 53 | 15.032 | −106.664 | −17.476 | 1.00 | 50.92 | O |
| ATOM | 323 | N | PHE | A | 54 | 13.235 | −105.679 | −18.372 | 1.00 | 48.21 | N |
| ATOM | 324 | CA | PHE | A | 54 | 12.605 | −105.366 | −17.092 | 1.00 | 46.22 | C |
| ATOM | 325 | CB | PHE | A | 54 | 12.328 | −103.857 | −16.995 | 1.00 | 42.82 | C |
| ATOM | 326 | CG | PHE | A | 54 | 13.572 | −103.020 | −16.881 | 1.00 | 37.35 | C |
| ATOM | 327 | CD1 | PHE | A | 54 | 14.309 | −102.687 | −18.017 | 1.00 | 33.49 | C |
| ATOM | 328 | CE1 | PHE | A | 54 | 15.460 | −101.932 | −17.918 | 1.00 | 32.31 | C |
| ATOM | 329 | CZ | PHE | A | 54 | 15.895 | −101.492 | −16.673 | 1.00 | 33.84 | C |
| ATOM | 330 | CE2 | PHE | A | 54 | 15.159 | −101.812 | −15.535 | 1.00 | 34.35 | C |
| ATOM | 331 | CD2 | PHE | A | 54 | 14.006 | −102.571 | −15.637 | 1.00 | 33.21 | C |
| ATOM | 332 | C | PHE | A | 54 | 11.330 | −106.144 | −16.821 | 1.00 | 48.54 | C |
| ATOM | 333 | O | PHE | A | 54 | 10.886 | −106.208 | −15.680 | 1.00 | 50.87 | O |
| ATOM | 334 | N | ILE | A | 55 | 10.760 | −106.760 | −17.851 | 1.00 | 55.42 | N |
| ATOM | 335 | CA | ILE | A | 55 | 9.471 | −107.450 | −17.701 | 1.00 | 62.63 | C |
| ATOM | 336 | CB | ILE | A | 55 | 8.343 | −106.732 | −18.503 | 1.00 | 62.09 | C |
| ATOM | 337 | CG1 | ILE | A | 55 | 8.119 | −105.305 | −17.980 | 1.00 | 64.38 | C |
| ATOM | 338 | CD1 | ILE | A | 55 | 8.775 | −104.207 | −18.808 | 1.00 | 68.23 | C |
| ATOM | 339 | CG2 | ILE | A | 55 | 7.035 | −107.502 | −18.424 | 1.00 | 65.00 | C |
| ATOM | 340 | C | ILE | A | 55 | 9.518 | −108.970 | −18.002 | 1.00 | 65.36 | C |
| ATOM | 341 | O | ILE | A | 55 | 9.058 | −109.779 | −17.179 | 1.00 | 66.61 | O |
| ATOM | 342 | N | GLN | A | 56 | 10.085 | −109.348 | −19.153 | 1.00 | 63.62 | N |
| ATOM | 343 | CA | GLN | A | 56 | 10.000 | −110.726 | −19.679 | 1.00 | 63.14 | C |
| ATOM | 344 | CB | GLN | A | 56 | 9.980 | −110.713 | −21.204 | 1.00 | 64.39 | C |
| ATOM | 345 | CG | GLN | A | 56 | 9.110 | −111.791 | −21.821 | 1.00 | 73.54 | C |
| ATOM | 346 | CD | GLN | A | 56 | 3.908 | −111.591 | −23.322 | 1.00 | 33.96 | C |
| ATOM | 347 | OE1 | GLN | A | 56 | 9.873 | −111.522 | −74.095 | 1.00 | 79.44 | O |
| ATOM | 348 | NE2 | GLN | A | 56 | 7.643 | −111.501 | −23.743 | 1.00 | 87.64 | N |
| ATOM | 349 | C | GLN | A | 56 | 11.117 | −111.651 | −19.204 | 1.00 | 63.08 | C |
| ATOM | 350 | O | GLN | A | 56 | 12.307 | −111.319 | −19.289 | 1.00 | 59.16 | O |
| ATOM | 351 | N | GLU | A | 57 | 10.726 | −112.823 | −18.709 | 1.00 | 65.40 | N |
| ATOM | 352 | CA | GLU | A | 57 | 11.700 | −113.830 | −18.289 | 1.00 | 65.46 | C |
| ATOM | 353 | CB | GLU | A | 57 | 11.117 | −114.763 | −17.215 | 1.00 | 65.85 | C |
| ATOM | 354 | CG | GLU | A | 57 | 11.475 | −114.298 | −15.800 | 1.00 | 70.75 | C |
| ATOM | 355 | CD | GLU | A | 57 | 10.558 | −114.823 | −14.695 | 1.00 | 78.94 | C |
| ATOM | 356 | OE1 | GLU | A | 57 | 10.823 | −114.532 | −13.499 | 1.00 | 72.74 | O |
| ATOM | 357 | OE2 | GLU | A | 57 | 9.568 | −115.522 | −15.007 | 1.00 | 83.72 | O |
| ATOM | 358 | C | GLU | A | 57 | 12.255 | −114.565 | −19.513 | 1.00 | 59.83 | C |
| ATOM | 359 | O | GLU | A | 57 | 11.530 | −114.803 | −20.469 | 1.00 | 59.43 | O |
| ATOM | 360 | N | PRO | A | 58 | 13.546 | −114.927 | −19.492 | 1.00 | 58.53 | N |
| ATOM | 361 | CA | PRO | A | 58 | 14.475 | −114.928 | −18.362 | 1.00 | 57.69 | C |
| ATOM | 362 | CB | PRO | A | 58 | 15.280 | −116.205 | −18.605 | 1.00 | 56.84 | C |
| ATOM | 363 | CG | PRO | A | 58 | 15.276 | −116.373 | −20.093 | 1.00 | 56.01 | C |
| ATOM | 364 | CD | PRO | A | 58 | 14.122 | −115.604 | −20.667 | 1.00 | 56.88 | C |
| ATOM | 365 | C | PRO | A | 58 | 15.431 | −113.746 | −18.232 | 1.00 | 59.55 | C |
| ATOM | 366 | O | PRO | A | 58 | 16.047 | −113.581 | −17.168 | 1.00 | 60.54 | O |
| ATOM | 367 | N | ALA | A | 59 | 15.563 | −112.947 | −19.292 | 1.00 | 56.51 | N |
| ATOM | 368 | CA | ALA | A | 59 | 16.532 | −111.845 | −19.342 | 1.00 | 52.12 | C |
| ATOM | 369 | CB | ALA | A | 59 | 16.473 | −111.159 | −20.692 | 1.00 | 54.93 | C |
| ATOM | 370 | C | ALA | A | 59 | 16.318 | −110.825 | −18.235 | 1.00 | 51.91 | C |
| ATOM | 371 | O | ALA | A | 59 | 17.256 | −110.140 | −17.808 | 1.00 | 58.57 | O |
| ATOM | 372 | N | LYS | A | 60 | 15.069 | −110.714 | −17.808 | 1.00 | 50.28 | N |
| ATOM | 373 | CA | LYS | A | 60 | 14.679 | −109.972 | −16.617 | 1.00 | 54.50 | C |
| ATOM | 374 | CB | LYS | A | 60 | 13.211 | −110.311 | −16.282 | 1.00 | 57.90 | C |
| ATOM | 375 | CG | LYS | A | 60 | 12.553 | −109.469 | −15.203 | 1.00 | 60.28 | C |
| ATOM | 376 | CD | LYS | A | 60 | 11.308 | −110.163 | −14.665 | 1.00 | 66.88 | C |
| ATOM | 377 | CE | LYS | A | 60 | 10.453 | −109.235 | −13.809 | 1.00 | 67.00 | C |
| ATOM | 378 | NZ | LYS | A | 60 | 11.143 | −108.787 | −12.567 | 1.00 | 64.21 | N |
| ATOM | 379 | C | LYS | A | 60 | 15.600 | −110.351 | −15.450 | 1.00 | 52.13 | C |
| ATOM | 380 | O | LYS | A | 60 | 16.160 | −109.472 | −14.791 | 1.00 | 46.29 | O |
| ATOM | 381 | N | ASN | A | 61 | 15.773 | −111.562 | −15.230 | 1.00 | 52.51 | N |
| ATOM | 382 | CA | ASN | A | 61 | 16.533 | −112.187 | −14.082 | 1.00 | 48.43 | C |
| ATOM | 383 | CB | ASN | A | 61 | 15.902 | −113.486 | −13.570 | 1.00 | 47.73 | C |
| ATOM | 384 | CG | ASN | A | 61 | 14.448 | −113.294 | −13.132 | 1.00 | 55.16 | C |
| ATOM | 385 | OD1 | ASN | A | 61 | 13.582 | −114.107 | −13.446 | 1.00 | 54.81 | O |
| ATOM | 386 | ND2 | ASN | A | 61 | 14.177 | −112.203 | −12.414 | 1.00 | 54.07 | N |
| ATOM | 387 | C | ASN | A | 61 | 15.042 | −112.227 | −14.310 | 1.00 | 45.33 | C |
| ATOM | 388 | O | ASN | A | 61 | 18.738 | −112.966 | −13.538 | 1.00 | 44.88 | O |
| ATOM | 389 | N | ARG | A | 61 | 18.541 | −111.703 | −15.368 | 1.00 | 45.55 | N |
| ATOM | 390 | CA | ARG | A | 62 | 19.979 | −111.576 | −15.589 | 1.00 | 45.86 | C |
| ATOM | 391 | CB | ARG | A | 62 | 20.217 | −110.464 | −16.599 | 1.00 | 44.90 | C |
| ATOM | 392 | CG | ARG | A | 62 | 21.666 | −110.163 | −16.908 | 1.00 | 45.11 | C |
| ATOM | 393 | CD | ARG | A | 62 | 21.748 | −109.975 | −18.403 | 1.00 | 44.72 | C |
| ATOM | 394 | NE | ARG | A | 62 | 21.667 | −108.582 | −18.748 | 1.00 | 45.87 | N |
| ATOM | 395 | CZ | ARG | A | 62 | 21.083 | −108.499 | −19.832 | 1.00 | 46.88 | C |
| ATOM | 396 | NH1 | ARG | A | 62 | 21.105 | −106.791 | −20.020 | 1.00 | 44.27 | N |
| ATOM | 397 | NH2 | ARG | A | 62 | 20.482 | −105.900 | −20.713 | 1.00 | 45.33 | N |
| ATOM | 398 | C | ARG | A | 62 | 20.742 | −111.269 | −14.279 | 1.00 | 45.84 | C |
| ATOM | 399 | O | ARG | A | 62 | 20.576 | −110.202 | −13.698 | 1.00 | 43.75 | O |
| ATOM | 400 | N | PRO | A | 63 | 21.588 | −112.206 | −13.817 | 1.00 | 48.31 | N |
| ATOM | 401 | CA | PRO | A | 63 | 22.335 | −111.925 | −12.595 | 1.00 | 45.27 | C |
| ATOM | 402 | CB | PRO | A | 63 | 23.221 | −113.164 | −12.434 | 1.00 | 50.06 | C |
| ATOM | 403 | CG | PRO | A | 63 | 22.494 | −114.251 | −13.169 | 1.00 | 51.29 | C |
| ATOM | 404 | CD | PRO | A | 63 | 21.843 | −113.567 | −14.331 | 1.00 | 49.24 | C |
| ATOM | 405 | C | PRO | A | 63 | 23.178 | −110.678 | −12.752 | 1.00 | 40.97 | C |
| ATOM | 406 | O | PRO | A | 63 | 23.586 | −116.350 | −13.864 | 1.00 | 41.78 | O |
| ATOM | 407 | N | GLY | A | 64 | 23.415 | −109.989 | −11.640 | 1.00 | 40.12 | N |
| ATOM | 408 | CA | GLY | A | 64 | 24.140 | −108.708 | −11.606 | 1.00 | 39.07 | C |
| ATOM | 409 | C | GLY | A | 64 | 25.629 | −108.968 | −11.513 | 1.00 | 39.76 | C |
| ATOM | 410 | O | GLY | A | 64 | 26.037 | −116.106 | −11.645 | 1.00 | 38.55 | O |
| ATOM | 411 | N | PRO | A | 65 | 26.455 | −107.919 | −11.290 | 1.00 | 42.20 | N |
| ATOM | 412 | CA | PRO | A | 65 | 26.094 | −106.494 | −11.098 | 1.00 | 40.32 | C |
| ATOM | 413 | CB | PRO | A | 65 | 27.435 | −105.841 | −10.730 | 1.00 | 42.83 | C |
| ATOM | 414 | CG | PRO | A | 65 | 28.479 | −106.732 | −11.332 | 1.00 | 39.26 | C |
| ATOM | 415 | CD | PRO | A | 65 | 27.921 | −108.129 | −11.253 | 1.00 | 37.51 | C |
| ATOM | 416 | C | PRO | A | 65 | 25.535 | −105.844 | −12.375 | 1.00 | 43.31 | C |
| ATOM | 417 | O | PRO | A | 65 | 25.979 | −106.169 | −13.478 | 1.00 | 44.09 | O |
| ATOM | 418 | N | GLN | A | 66 | 24.587 | −104.925 | −12.226 | 1.00 | 43.24 | N |
| ATOM | 419 | CA | GLN | A | 66 | 24.001 | −104.244 | −13.373 | 1.00 | 40.88 | C |
| ATOM | 420 | CB | GLN | A | 66 | 22.663 | −103.591 | −13.016 | 1.00 | 37.29 | C |
| ATOM | 421 | CG | GLN | A | 66 | 21.553 | −104.555 | −12.622 | 1.00 | 36.54 | C |
| ATOM | 422 | CD | GLN | A | 66 | 21.356 | −105.711 | −13.586 | 1.00 | 39.92 | C |
| ATOM | 423 | CE1 | GLN | A | 66 | 21.351 | −105.535 | −14.807 | 1.00 | 39.83 | O |
| ATOM | 424 | NE2 | GLN | A | 66 | 21.176 | −106.922 | −13.029 | 1.00 | 40.52 | N |
| ATOM | 425 | C | GLN | A | 66 | 24.961 | −103.226 | −13.970 | 1.00 | 42.27 | C |
| ATOM | 426 | C | GLN | A | 66 | 24.842 | −102.879 | −15.151 | 1.00 | 43.99 | O |
| ATOM | 427 | N | THR | A | 67 | 25.907 | −102.748 | −13.156 | 1.00 | 38.70 | N |
| ATOM | 428 | CA | THR | A | 67 | 26.984 | −101.888 | −13.651 | 1.00 | 37.43 | C |
| ATOM | 429 | CB | THR | A | 67 | 26.519 | −100.438 | −13.891 | 1.00 | 40.35 | C |
| ATOM | 430 | CG1 | THR | A | 67 | 27.584 | −99.687 | −14.482 | 1.00 | 40.53 | O |
| ATOM | 431 | CG2 | THR | A | 67 | 26.118 | −99.756 | −12.571 | 1.00 | 41.63 | C |
| ATOM | 432 | C | THR | A | 67 | 28.194 | −101.672 | −12.725 | 1.00 | 37.85 | C |
| ATOM | 433 | O | THR | A | 67 | 28.264 | −102.603 | −11.753 | 1.00 | 40.39 | O |
| ATOM | 434 | N | ARG | A | 68 | 29.123 | −100.995 | −13.043 | 1.00 | 38.59 | N |
| ATOM | 435 | CA | ARG | A | 68 | 30.343 | −100.783 | −12.302 | 1.00 | 43.86 | C |
| ATOM | 436 | CB | ARG | A | 68 | 31.367 | −100.163 | −13.267 | 1.00 | 46.57 | C |
| ATOM | 437 | CG | ARG | A | 68 | 32.113 | −98.921 | −12.837 | 1.00 | 53.83 | C |
| ATOM | 438 | CD | ARG | A | 68 | 32.735 | −98.243 | −14.056 | 1.00 | 55.50 | C |
| ATOM | 439 | NE | ARG | A | 68 | 33.449 | −99.189 | −14.918 | 1.00 | 53.04 | N |
| ATOM | 440 | CZ | ARG | A | 68 | 33.776 | −98.965 | −16.190 | 1.00 | 55.82 | C |
| ATOM | 441 | NH1 | ARG | A | 68 | 33.447 | −97.823 | −16.790 | 1.00 | 55.96 | N |
| ATOM | 442 | NH2 | ARG | A | 68 | 34.424 | −99.901 | −16.876 | 1.00 | 56.06 | N |
| ATOM | 443 | C | ARG | A | 68 | 30.108 | −99.955 | −11.032 | 1.00 | 46.99 | C |
| ATOM | 444 | O | ARG | A | 68 | 29.296 | −99.029 | −11.003 | 1.00 | 51.17 | O |
| ATOM | 445 | N | SER | A | 69 | 30.857 | −100.301 | −9.995 | 1.00 | 47.24 | N |
| ATOM | 446 | CA | SER | A | 69 | 30.616 | −99.870 | −8.635 | 1.00 | 44.29 | C |
| ATOM | 447 | CB | SER | A | 69 | 31.395 | −100.816 | −7.702 | 1.00 | 42.30 | C |
| ATOM | 448 | OG | SER | A | 69 | 31.424 | −100.376 | −6.360 | 1.00 | 42.69 | O |
| ATOM | 449 | C | SER | A | 69 | 30.956 | −98.398 | −8.356 | 1.00 | 46.48 | C |
| ATOM | 450 | O | SER | A | 69 | 31.976 | −97.849 | −8.902 | 1.00 | 45.39 | O |
| ATOM | 451 | N | ASP | A | 70 | 30.144 | −97.765 | −7.505 | 1.00 | 46.29 | N |
| ATOM | 452 | CA | ASP | A | 70 | 30.430 | −96.400 | −7.012 | 1.00 | 46.65 | C |
| ATOM | 453 | CB | ASP | A | 70 | 29.279 | −95.836 | −6.165 | 1.00 | 45.40 | C |
| ATOM | 454 | CG | ASP | A | 70 | 28.906 | −96.711 | −4.964 | 1.00 | 47.56 | C |
| ATOM | 455 | OD1 | ASP | A | 70 | 29.068 | −97.955 | −5.004 | 1.00 | 48.85 | O |
| ATOM | 456 | OD2 | ASP | A | 70 | 28.408 | −96.133 | −3.969 | 1.00 | 47.88 | O |
| ATOM | 457 | C | ASP | A | 70 | 31.735 | −96.273 | −6.234 | 1.00 | 44.54 | C |
| ATOM | 458 | O | ASP | A | 70 | 32.321 | −95.184 | −6.160 | 1.00 | 44.00 | O |
| ATOM | 459 | N | LEU | A | 71 | 32.204 | −97.381 | −5.670 | 1.00 | 44.27 | N |
| ATOM | 460 | CA | LEU | A | 71 | 33.440 | −97.332 | −4.875 | 1.00 | 44.84 | C |
| ATOM | 461 | CB | LEU | A | 71 | 33.676 | −98.641 | −4.131 | 1.00 | 44.06 | C |
| ATOM | 462 | CG | LEU | A | 71 | 32.683 | −98.903 | −2.994 | 1.00 | 44.10 | C |
| ATOM | 463 | CD1 | LEU | A | 71 | 32.843 | −100.317 | −2.434 | 1.00 | 41.56 | C |
| ATOM | 464 | CD2 | LEU | A | 71 | 32.822 | −97.861 | −1.888 | 1.00 | 42.69 | C |
| ATOM | 465 | C | LEU | A | 71 | 34.655 | −96.938 | −5.696 | 1.00 | 42.04 | C |
| ATOM | 466 | O | LEU | A | 71 | 35.623 | −96.417 | −5.154 | 1.00 | 43.82 | O |
| ATOM | 467 | N | LEU | A | 72 | 34.561 | −97.133 | −7.011 | 1.00 | 44.56 | N |
| ATOM | 468 | CA | LEU | A | 72 | 35.649 | −96.875 | −7.951 | 1.00 | 40.67 | C |
| ATOM | 469 | CB | LEU | A | 72 | 35.302 | −97.365 | −9.339 | 1.00 | 38.29 | C |
| ATOM | 470 | CG | LEU | A | 72 | 35.401 | −98.875 | −3.481 | 1.00 | 40.04 | C |
| ATOM | 471 | CD1 | LEU | A | 72 | 34.797 | −99.311 | −10.811 | 1.00 | 41.21 | C |
| ATOM | 472 | CD2 | LEU | A | 72 | 36.870 | −99.297 | −9.374 | 1.00 | 42.71 | C |
| ATOM | 473 | C | LEU | A | 72 | 36.116 | −95.443 | −8.017 | 1.00 | 43.86 | C |
| ATOM | 474 | O | LEU | A | 72 | 37.216 | −95.184 | −8.512 | 1.00 | 48.77 | O |
| ATOM | 475 | N | LEU | A | 73 | 35.314 | −94.514 | −7.504 | 1.00 | 44.68 | N |
| ATOM | 476 | CA | LEU | A | 73 | 35.743 | −93.115 | −7.381 | 1.00 | 44.86 | C |
| ATOM | 477 | CB | LEU | A | 73 | 33.128 | −92.247 | −8.478 | 1.00 | 44.24 | C |
| ATOM | 478 | CG | LEU | A | 73 | 35.469 | −92.407 | −9.959 | 1.00 | 45.34 | C |
| ATOM | 479 | CD1 | LEU | A | 73 | 34.686 | −91.367 | −10.772 | 1.00 | 39.88 | C |
| ATOM | 480 | CD2 | LEU | A | 73 | 36.975 | −92.240 | −10.171 | 1.00 | 43.21 | C |
| ATOM | 481 | C | LEU | A | 73 | 33.361 | −92.533 | −6.034 | 1.00 | 47.18 | C |
| ATOM | 482 | O | LEU | A | 73 | 34.381 | −92.972 | −5.408 | 1.00 | 49.64 | O |
| ATOM | 483 | N | SER | A | 74 | 36.138 | −91.547 | −5.592 | 1.00 | 46.28 | N |
| ATOM | 484 | CA | SER | A | 74 | 33.748 | −90.716 | −4.461 | 1.00 | 47.78 | C |
| ATOM | 485 | CB | SER | A | 74 | 36.853 | −89.699 | −4.130 | 1.00 | 46.92 | C |
| ATOM | 486 | OG | SER | A | 74 | 36.930 | −88.652 | −5.090 | 1.00 | 45.43 | O |
| ATOM | 487 | C | SER | A | 74 | 34.428 | −89.970 | −4.713 | 1.00 | 49.61 | C |
| ATOM | 488 | O | SER | A | 74 | 34.094 | −89.596 | −5.848 | 1.00 | 48.53 | O |
| ATOM | 489 | N | GLY | A | 75 | 33.696 | −89.742 | −3.633 | 1.00 | 52.56 | N |
| ATOM | 490 | CA | GLY | A | 75 | 32.548 | −88.849 | −3.655 | 1.00 | 51.82 | C |
| ATOM | 491 | C | GLY | A | 75 | 32.866 | −87.556 | −4.367 | 1.00 | 51.97 | C |
| ATOM | 492 | O | GLY | A | 75 | 32.105 | −87.130 | −5.225 | 1.00 | 51.94 | O |
| ATOM | 493 | N | ARG | A | 76 | 34.006 | −86.944 | −4.041 | 1.00 | 54.06 | N |
| ATOM | 494 | CA | ARG | A | 76 | 34.371 | −85.582 | −4.672 | 1.00 | 53.09 | C |
| ATOM | 495 | CB | ARG | A | 76 | 35.629 | −85.344 | −4.065 | 1.00 | 57.69 | C |
| ATOM | 496 | CG | ARG | A | 76 | 36.165 | −83.914 | −4.952 | 1.00 | 65.78 | C |
| ATOM | 497 | CD | ARG | A | 76 | 37.274 | −83.357 | −4.363 | 1.00 | 71.53 | C |
| ATOM | 498 | NE | ARG | A | 76 | 36.915 | −82.406 | −3.097 | 1.00 | 82.09 | N |
| ATOM | 499 | CZ | ARG | A | 76 | 35.916 | −81.537 | −2.915 | 1.00 | 84.58 | C |
| ATOM | 500 | NH1 | ARG | A | 76 | 35.098 | −81.193 | −3.909 | 1.00 | 83.53 | C |
| ATOM | 501 | NH2 | ARG | A | 76 | 35.724 | −81.924 | −1.705 | 1.00 | 86.27 | N |
| ATOM | 502 | C | ARG | A | 76 | 34.539 | −85.832 | −6.178 | 1.00 | 52.74 | C |
| ATOM | 503 | O | ARG | A | 76 | 34.113 | −84.956 | −6.930 | 1.00 | 55.31 | O |
| ATOM | 504 | N | ASP | A | 77 | 35.154 | −86.928 | −6.616 | 1.00 | 49.27 | N |
| ATOM | 505 | CA | ASP | A | 77 | 35.384 | −87.143 | −8.048 | 1.00 | 48.71 | C |
| ATOM | 506 | CB | ASP | A | 77 | 36.310 | −88.348 | −8.296 | 1.00 | 47.87 | C |
| ATOM | 507 | CG | ASP | A | 77 | 37.810 | −88.317 | −8.072 | 1.00 | 52.71 | C |
| ATOM | 508 | OD1 | ASP | A | 77 | 38.182 | −86.837 | −7.865 | 1.00 | 54.74 | O |
| ATOM | 509 | OD2 | ASP | A | 77 | 38.628 | −88.956 | −8.105 | 1.00 | 50.81 | O |
| ATOM | 510 | C | ASP | A | 77 | 34.058 | −87.299 | −8.811 | 1.00 | 45.07 | C |
| ATOM | 511 | O | ASP | A | 77 | 33.892 | −86.777 | −9.930 | 1.00 | 42.37 | O |
| ATOM | 512 | N | TRP | A | 78 | 33.122 | −88.010 | −8.196 | 1.00 | 39.30 | N |
| ATOM | 513 | CA | TRP | A | 78 | 31.804 | −88.172 | −8.768 | 1.00 | 39.03 | C |
| ATOM | 514 | CB | TRP | A | 78 | 30.986 | −89.145 | −7.931 | 1.00 | 37.92 | C |
| ATOM | 515 | CG | TRP | A | 78 | 31.330 | −90.623 | −8.072 | 1.00 | 40.66 | C |
| ATOM | 516 | CD1 | TRP | A | 78 | 31.738 | −91.464 | −7.071 | 1.00 | 40.56 | C |
| ATOM | 517 | NE1 | TRP | A | 78 | 31.922 | −92.747 | −7.559 | 1.00 | 40.28 | N |
| ATOM | 518 | CE2 | TRP | A | 78 | 31.636 | −92.759 | −8.901 | 1.00 | 40.56 | C |
| ATOM | 519 | CD2 | TRP | A | 78 | 31.258 | −91.440 | −9.268 | 1.00 | 42.84 | C |
| ATOM | 520 | CE3 | TRP | A | 78 | 30.917 | −91.185 | −10.606 | 1.00 | 41.86 | C |
| ATOM | 521 | CZ3 | TRP | A | 78 | 30.965 | −92.248 | −11.526 | 1.00 | 41.10 | C |
| ATOM | 522 | CH2 | TRP | A | 78 | 31.329 | −93.545 | −11.121 | 1.00 | 39.77 | C |
| ATOM | 523 | CZ2 | TRP | A | 78 | 31.670 | −93.818 | −9.819 | 1.00 | 39.05 | C |
| ATOM | 524 | C | TRP | A | 78 | 31.089 | −86.307 | −8.867 | 1.00 | 41.09 | C |
| ATOM | 525 | O | TRP | A | 78 | 30.591 | −86.447 | −9.928 | 1.00 | 42.27 | O |
| ATOM | 526 | N | ASN | A | 79 | 31.075 | −86.049 | −7.771 | 1.00 | 40.49 | N |
| ATOM | 527 | CA | ASN | A | 79 | 30.484 | −84.718 | −7.724 | 1.00 | 41.29 | C |
| ATOM | 528 | CB | ASN | A | 79 | 30.723 | −84.089 | −6.354 | 1.00 | 43.43 | C |
| ATOM | 529 | CG | ASN | A | 79 | 29.917 | −84.729 | −5.258 | 1.00 | 46.48 | C |
| ATOM | 530 | CD1 | ASN | A | 79 | 29.054 | −85.588 | −5.499 | 1.00 | 45.62 | O |
| ATOM | 531 | ND2 | ASN | A | 79 | 30.197 | −84.309 | −4.022 | 1.00 | 47.42 | N |
| ATOM | 532 | C | ASN | A | 79 | 31.029 | −83.710 | −8.725 | 1.00 | 43.97 | C |
| ATOM | 533 | O | ASN | A | 79 | 30.357 | −82.722 | −9.044 | 1.00 | 46.17 | O |
| ATOM | 534 | N | THR | A | 80 | 32.255 | −83.906 | −9.189 | 1.00 | 44.16 | N |
| ATOM | 535 | CA | THR | A | 80 | 32.928 | −82.810 | −9.893 | 1.00 | 45.69 | C |
| ATOM | 536 | CB | THR | A | 80 | 34.016 | −82.135 | −9.025 | 1.00 | 44.75 | C |
| ATOM | 537 | OG1 | THR | A | 80 | 34.817 | −83.136 | −8.400 | 1.00 | 48.06 | O |
| ATOM | 538 | OG2 | THR | A | 80 | 33.398 | −81.215 | −7.953 | 1.00 | 42.20 | C |
| ATOM | 539 | C | THR | A | 80 | 33.552 | −83.168 | −11.225 | 1.00 | 44.18 | C |
| ATOM | 540 | O | THR | A | 80 | 33.728 | −82.305 | −12.082 | 1.00 | 45.43 | O |
| ATOM | 541 | N | LEU | A | 81 | 33.883 | −84.431 | −11.413 | 1.00 | 45.35 | N |
| ATOM | 542 | CA | LEU | A | 81 | 34.601 | −84.792 | −12.621 | 1.00 | 49.41 | C |
| ATOM | 543 | CB | LEU | A | 81 | 35.901 | −85.554 | −12.278 | 1.00 | 53.74 | C |
| ATOM | 544 | CG | LEU | A | 81 | 36.872 | −84.910 | −11.264 | 1.00 | 52.94 | C |
| ATOM | 545 | CD1 | LEU | A | 81 | 38.085 | −85.808 | −11.079 | 1.00 | 57.04 | C |
| ATOM | 546 | CD2 | LEU | A | 81 | 37.300 | −83.495 | −11.648 | 1.00 | 49.48 | C |
| ATOM | 547 | C | LEU | A | 81 | 33.761 | −85.530 | −13.664 | 1.00 | 49.12 | C |
| ATOM | 548 | O | LEU | A | 81 | 34.169 | −85.630 | −14.818 | 1.00 | 55.36 | O |
| ATOM | 549 | N | ILE | A | 82 | 32.589 | −86.021 | −13.272 | 1.00 | 44.77 | N |
| ATOM | 550 | CA | ILE | A | 82 | 31.809 | −86.916 | −14.134 | 1.00 | 43.01 | C |
| ATOM | 551 | CB | ILE | A | 82 | 31.584 | −88.271 | −13.446 | 1.00 | 40.95 | C |
| ATOM | 552 | CG1 | ILE | A | 82 | 37.917 | −88.826 | −12.935 | 1.00 | 42.22 | C |
| ATOM | 553 | CD1 | ILE | A | 82 | 33.831 | −89.396 | −14.004 | 1.00 | 37.64 | C |
| ATOM | 554 | CG2 | ILE | A | 82 | 30.850 | −89.243 | −14.373 | 1.00 | 38.24 | C |
| ATOM | 555 | C | ILE | A | 82 | 30.444 | −86.379 | −14.571 | 1.00 | 43.39 | C |
| ATOM | 556 | O | ILE | A | 82 | 29.647 | −85.915 | −13.765 | 1.00 | 45.91 | O |
| ATOM | 557 | N | VAL | A | 83 | 30.190 | −86.477 | −15.864 | 1.00 | 44.03 | N |
| ATOM | 558 | CA | VAL | A | 83 | 28.899 | −86.187 | −16.442 | 1.00 | 41.06 | C |
| ATOM | 559 | CB | VAL | A | 83 | 29.031 | −85.121 | −17.545 | 1.00 | 40.83 | C |
| ATOM | 560 | CG1 | VAL | A | 83 | 27.765 | −85.043 | −18.385 | 1.00 | 41.55 | C |
| ATOM | 561 | CG2 | VAL | A | 83 | 29.373 | −83.764 | −16.932 | 1.00 | 38.24 | C |
| ATOM | 562 | C | VAL | A | 83 | 28.392 | −87.491 | −17.020 | 1.00 | 39.55 | C |
| ATOM | 563 | O | VAL | A | 83 | 29.057 | −88.093 | −17.870 | 1.00 | 40.68 | O |
| ATOM | 564 | N | GLY | A | 84 | 27.231 | −87.938 | −16.538 | 1.00 | 39.83 | N |
| ATOM | 565 | CA | GLY | A | 84 | 26.572 | −89.146 | −17.047 | 1.00 | 35.63 | C |
| ATOM | 566 | C | GLY | A | 84 | 26.041 | −88.982 | −18.469 | 1.00 | 34.98 | C |
| ATOM | 567 | O | GLY | A | 84 | 25.905 | −87.870 | −18.968 | 1.00 | 34.30 | O |
| ATOM | 568 | N | LYS | A | 85 | 25.747 | −90.102 | −19.122 | 1.00 | 37.85 | N |
| ATOM | 569 | CA | LYS | A | 85 | 25.154 | −90.105 | −20.457 | 1.00 | 40.47 | C |
| ATOM | 570 | CB | LYS | A | 85 | 26.172 | −90.471 | −21.535 | 1.00 | 39.59 | C |
| ATOM | 571 | CG | LYS | A | 85 | 27.344 | −89.521 | −21.677 | 1.00 | 44.46 | C |
| ATOM | 572 | CD | LYS | A | 85 | 28.407 | −90.096 | −22.610 | 1.00 | 42.32 | C |
| ATOM | 573 | CE | LYS | A | 85 | 28.196 | −89.688 | −24.052 | 1.00 | 45.28 | C |
| ATOM | 574 | NZ | LYS | A | 85 | 28.747 | −90.699 | −24.993 | 1.00 | 53.62 | N |
| ATOM | 575 | C | LYS | A | 85 | 24.096 | −91.178 | −20.443 | 1.00 | 41.45 | C |
| ATOM | 576 | O | LYS | A | 85 | 24.342 | −92.282 | −19.962 | 1.00 | 39.06 | O |
| ATOM | 577 | N | LEU | A | 86 | 22.920 | −90.854 | −20.975 | 1.00 | 40.31 | N |
| ATOM | 578 | CA | LEU | A | 86 | 21.870 | −91.863 | −21.139 | 1.00 | 39.24 | C |
| ATOM | 579 | CB | LEU | A | 86 | 20.569 | −91.200 | −21.624 | 1.00 | 34.16 | C |
| ATOM | 580 | CG | LEU | A | 86 | 19.352 | −92.117 | −21.721 | 1.00 | 35.27 | C |
| ATOM | 581 | CD1 | LEU | A | 86 | 18.907 | −92.488 | −20.312 | 1.00 | 33.98 | C |
| ATOM | 582 | CD2 | LEU | A | 86 | 18.221 | −91.466 | −22.517 | 1.00 | 35.22 | C |
| ATOM | 583 | C | LEU | A | 86 | 22.332 | −92.944 | −22.140 | 1.00 | 39.95 | C |
| ATOM | 584 | O | LEU | A | 86 | 22.945 | −92.636 | −23.156 | 1.00 | 37.07 | O |
| ATOM | 585 | N | SER | A | 87 | 22.036 | −94.205 | −21.864 | 1.00 | 39.36 | N |
| ATOM | 586 | CA | SER | A | 87 | 22.379 | −95.258 | −22.815 | 1.00 | 41.04 | C |
| ATOM | 587 | CB | SER | A | 87 | 22.128 | −96.622 | −22.202 | 1.00 | 39.59 | C |
| ATOM | 588 | OG | SER | A | 87 | 23.009 | −96.816 | −21.114 | 1.00 | 44.82 | O |
| ATOM | 589 | C | SER | A | 87 | 21.638 | −95.104 | −24.153 | 1.00 | 43.52 | C |
| ATOM | 590 | O | SER | A | 87 | 20.433 | −94.860 | −24.187 | 1.00 | 50.28 | O |
| ATOM | 591 | N | PRO | A | 88 | 22.358 | −95.282 | −25.263 | 1.00 | 43.27 | N |
| ATOM | 592 | CA | PRO | A | 88 | 21.841 | −94.836 | −26.540 | 1.00 | 40.91 | C |
| ATOM | 593 | CB | PRO | A | 88 | 23.072 | −94.890 | −27.437 | 1.00 | 41.29 | C |
| ATOM | 594 | CG | PRO | A | 88 | 23.812 | −96.076 | −26.912 | 1.00 | 44.51 | C |
| ATOM | 595 | CD | PRO | A | 88 | 23.627 | −96.026 | −25.417 | 1.00 | 43.40 | C |
| ATOM | 596 | C | PRO | A | 88 | 20.769 | −95.729 | −27.108 | 1.00 | 37.53 | C |
| ATOM | 597 | O | PRO | A | 88 | 20.233 | −95.385 | −28.136 | 1.00 | 39.86 | O |
| ATOM | 598 | N | TRP | A | 89 | 20.486 | −96.858 | −26.456 | 1.00 | 34.83 | N |
| ATOM | 599 | CA | TRP | A | 89 | 19.426 | −97.771 | −26.858 | 1.00 | 32.45 | C |
| ATOM | 600 | CB | TRP | A | 89 | 19.767 | −99.224 | −26.501 | 1.00 | 32.12 | C |
| ATOM | 601 | CG | TRP | A | 89 | 20.030 | −99.498 | −25.007 | 1.00 | 33.71 | C |
| ATOM | 602 | CD1 | TRP | A | 89 | 21.243 | −99.414 | −24.351 | 1.00 | 34.05 | C |
| ATOM | 603 | NE1 | TRP | A | 89 | 21.080 | −99.741 | −23.021 | 1.00 | 34.80 | N |
| ATOM | 604 | CE2 | TRP | A | 89 | 19.767 | −100.056 | −22.795 | 1.00 | 31.01 | C |
| ATOM | 605 | CD2 | TRP | A | 89 | 19.076 | −99.923 | −24.019 | 1.00 | 31.97 | C |
| ATOM | 606 | CE3 | TRP | A | 89 | 17.686 | −100.168 | −24.043 | 1.00 | 32.69 | C |
| ATOM | 607 | CZ3 | TRP | A | 89 | 17.047 | −100.554 | −22.870 | 1.00 | 29.34 | C |
| ATOM | 608 | CH2 | TRP | A | 89 | 17.759 | −100.680 | −21.667 | 1.00 | 30.46 | C |
| ATOM | 609 | CZ2 | TRP | A | 89 | 19.123 | −100.432 | −21.610 | 1.00 | 34.97 | C |
| ATOM | 610 | C | TRP | A | 89 | 18.104 | −97.425 | −26.184 | 1.00 | 37.08 | C |
| ATOM | 611 | O | TRP | A | 89 | 17.063 | −98.012 | −26.501 | 1.00 | 34.83 | O |
| ATOM | 612 | N | ILE | A | 90 | 18.165 | −96.524 | −25.205 | 1.00 | 38.04 | N |
| ATOM | 613 | CA | ILE | A | 90 | 16.992 | −96.133 | −24.467 | 1.00 | 38.01 | C |
| ATOM | 614 | CB | ILE | A | 90 | 17.374 | −95.467 | −23.136 | 1.00 | 40.38 | C |
| ATOM | 615 | CG1 | ILE | A | 90 | 18.017 | −96.516 | −22.202 | 1.00 | 35.71 | C |
| ATOM | 616 | CD1 | ILE | A | 90 | 18.817 | −95.924 | −21.073 | 1.00 | 32.82 | C |
| ATOM | 617 | CG2 | ILE | A | 90 | 16.148 | −94.795 | −22.506 | 1.00 | 36.71 | C |
| ATOM | 618 | C | ILE | A | 90 | 16.200 | −95.214 | −25.370 | 1.00 | 38.75 | C |
| ATOM | 619 | O | ILE | A | 90 | 16.682 | −94.148 | −25.740 | 1.00 | 38.81 | O |
| ATOM | 620 | N | ARG | A | 91 | 15.018 | −95.572 | −25.779 | 1.00 | 35.36 | N |
| ATOM | 621 | CA | ARG | A | 91 | 14.181 | −94.335 | −26.725 | 1.00 | 35.49 | C |
| ATOM | 622 | CB | ARG | A | 91 | 14.214 | −95.599 | −28.114 | 1.00 | 33.88 | C |
| ATOM | 623 | CG | ARG | A | 91 | 15.538 | −95.529 | −28.834 | 1.00 | 35.61 | C |
| ATOM | 624 | CD | ARG | A | 91 | 15.761 | −94.206 | −29.550 | 1.00 | 38.15 | C |
| ATOM | 625 | NE | ARG | A | 91 | 16.108 | −93.140 | −28.615 | 1.00 | 42.99 | N |
| ATOM | 626 | CZ | ARG | A | 91 | 16.223 | −91.248 | −28.936 | 1.00 | 44.08 | C |
| ATOM | 627 | NH1 | ARG | A | 91 | 16.531 | −90.261 | −27.992 | 1.00 | 38.62 | N |
| ATOM | 628 | NH2 | ARG | A | 91 | 16.038 | −91.437 | −30.195 | 1.00 | 45.66 | N |
| ATOM | 629 | C | ARG | A | 91 | 12.738 | −94.367 | −26.191 | 1.00 | 36.06 | C |
| ATOM | 630 | O | ARG | A | 91 | 11.879 | −95.592 | −26.551 | 1.00 | 31.97 | O |
| ATOM | 631 | N | PRO | A | 92 | 12.471 | −93.393 | −25.307 | 1.00 | 35.78 | N |
| ATOM | 632 | CA | PRO | A | 92 | 11.159 | −93.306 | −24.628 | 1.00 | 34.77 | C |
| ATOM | 633 | CB | PRO | A | 92 | 11.347 | −92.556 | −23.633 | 1.00 | 36.52 | C |
| ATOM | 634 | CG | PRO | A | 92 | 12.824 | −92.374 | −23.586 | 1.00 | 36.21 | C |
| ATOM | 635 | CD | PRO | A | 92 | 13.427 | −92.466 | −24.859 | 1.00 | 31.95 | C |
| ATOM | 636 | C | PRO | A | 92 | 10.014 | −93.484 | −25.586 | 1.00 | 36.06 | C |
| ATOM | 637 | O | PRO | A | 92 | 8.829 | −93.640 | −25.244 | 1.00 | 40.20 | O |
| ATOM | 638 | N | ASP | A | 93 | 10.365 | −93.648 | −26.784 | 1.00 | 36.61 | N |
| ATOM | 639 | CA | ASP | A | 93 | 9.388 | −92.602 | −27.759 | 1.00 | 34.98 | C |
| ATOM | 640 | CB | ASP | A | 93 | 9.639 | −91.152 | −28.108 | 1.00 | 33.74 | C |
| ATOM | 641 | CG | ASP | A | 93 | 9.159 | −90.245 | −27.019 | 1.00 | 36.52 | C |
| ATOM | 642 | OD1 | ASP | A | 93 | 7.899 | −90.083 | −26.895 | 1.00 | 34.93 | O |
| ATOM | 643 | OD2 | ASP | A | 93 | 10.031 | −89.751 | −26.255 | 1.00 | 32.77 | O |
| ATOM | 644 | C | ASP | A | 93 | 9.357 | −93.459 | −28.990 | 1.00 | 35.92 | C |
| ATOM | 645 | O | ASP | A | 93 | 8.950 | −93.018 | −30.055 | 1.00 | 38.67 | O |
| ATOM | 646 | N | SER | A | 94 | 9.804 | −94.696 | −28.834 | 1.00 | 37.95 | N |
| ATOM | 647 | CA | SER | A | 94 | 9.739 | −95.673 | −79.899 | 1.00 | 36.54 | C |
| ATOM | 648 | CB | SER | A | 94 | 10.580 | −96.879 | −29.498 | 1.00 | 37.89 | C |
| ATOM | 649 | OG | SER | A | 94 | 9.987 | −98.105 | −29.871 | 1.00 | 39.43 | O |
| ATOM | 650 | C | SER | A | 94 | 8.284 | −96.080 | −30.124 | 1.00 | 37.15 | C |
| ATOM | 651 | O | SER | A | 94 | 7.449 | −95.963 | −29.231 | 1.00 | 32.73 | O |
| ATOM | 652 | N | LYS | A | 95 | 8.013 | −96.579 | −31.318 | 1.00 | 41.15 | N |
| ATOM | 653 | CA | LYS | A | 95 | 6.674 | −96.964 | −31.721 | 1.00 | 46.86 | C |
| ATOM | 654 | CB | LYS | A | 95 | 6.537 | −96.870 | −33.256 | 1.00 | 54.28 | C |
| ATOM | 655 | CG | LYS | A | 95 | 7.113 | −95.610 | −33.906 | 1.00 | 60.72 | C |
| ATOM | 656 | CD | LYS | A | 95 | 6.031 | −94.712 | −34.501 | 1.00 | 62.96 | C |
| ATOM | 657 | CE | LYS | A | 95 | 5.228 | −93.984 | −33.434 | 1.00 | 64.26 | C |
| ATOM | 658 | NZ | LYS | A | 95 | 5.874 | −92.709 | −33.010 | 1.00 | 64.44 | N |
| ATOM | 659 | C | LYS | A | 95 | 6.384 | −98.387 | −31.288 | 1.00 | 44.83 | C |
| ATOM | 660 | O | LYS | A | 95 | 5.285 | −98.886 | −31.511 | 1.00 | 43.96 | O |
| ATOM | 661 | N | VAL | A | 96 | 7.377 | −99.061 | −30.713 | 1.00 | 41.51 | N |
| ATOM | 662 | CA | VAL | A | 96 | 7.194 | −100.443 | −30.294 | 1.00 | 40.98 | C |
| ATOM | 663 | CB | VAL | A | 96 | 8.422 | −101.327 | −30.641 | 1.00 | 44.98 | C |
| ATOM | 664 | CG1 | VAL | A | 96 | 8.317 | −102.707 | −30.003 | 1.00 | 42.48 | C |
| ATOM | 665 | CG2 | VAL | A | 96 | 8.582 | −101.472 | −32.145 | 1.00 | 45.47 | C |
| ATOM | 666 | C | VAL | A | 96 | 6.990 | −100.422 | −28.802 | 1.00 | 41.68 | C |
| ATOM | 667 | O | VAL | A | 96 | 7.878 | −99.973 | −28.059 | 1.00 | 41.02 | O |
| ATOM | 668 | N | GLU | A | 97 | 5.840 | −100.924 | −28.359 | 1.00 | 41.87 | N |
| ATOM | 669 | CA | GLU | A | 97 | 5.487 | −100.855 | −26.945 | 1.00 | 44.03 | C |
| ATOM | 670 | CB | GLU | A | 97 | 4.084 | −101.387 | −26.682 | 1.00 | 45.55 | C |
| ATOM | 671 | CG | GLU | A | 97 | 3.562 | −100.911 | −25.341 | 1.00 | 50.55 | C |
| ATOM | 672 | CD | GLU | A | 97 | 2.437 | −101.760 | −24.807 | 1.00 | 55.62 | C |
| ATOM | 673 | OE1 | GLU | A | 97 | 1.289 | −101.606 | −25.283 | 1.00 | 59.54 | O |
| ATOM | 674 | OE2 | GLU | A | 97 | 2.710 | −102.574 | −23.900 | 1.00 | 57.62 | O |
| ATOM | 675 | C | GLU | A | 97 | 6.503 | −101.491 | −23.976 | 1.00 | 42.67 | C |
| ATOM | 676 | O | GLU | A | 97 | 6.834 | −100.887 | −24.932 | 1.00 | 42.88 | O |
| ATOM | 677 | N | LYS | A | 98 | 7.010 | −102.676 | −26.311 | 1.00 | 39.77 | N |
| ATOM | 678 | CA | LYS | A | 98 | 7.949 | −103.351 | −25.404 | 1.00 | 40.70 | C |
| ATOM | 679 | CB | LYS | A | 98 | 8.251 | −104.779 | −25.847 | 1.00 | 43.84 | C |
| ATOM | 680 | CG | LYS | A | 98 | 7.194 | −105.829 | −25.546 | 1.00 | 44.99 | C |
| ATOM | 681 | CD | LYS | A | 98 | 7.249 | −106.884 | −26.650 | 1.00 | 50.67 | C |
| ATOM | 682 | CE | LYS | A | 98 | 6.555 | −108.190 | −26.278 | 1.00 | 56.41 | C |
| ATOM | 683 | NZ | LYS | A | 98 | 7.520 | −109.242 | −25.830 | 1.00 | 58.24 | N |
| ATOM | 684 | C | LYS | A | 98 | 0.241 | −102.550 | −75.237 | 1.00 | 38.72 | C |
| ATOM | 685 | O | LYS | A | 98 | 9.797 | −102.501 | −24.136 | 1.00 | 42.48 | O |
| ATOM | 686 | N | ILE | A | 99 | 9.678 | −101.902 | −26.314 | 1.00 | 37.90 | N |
| ATOM | 687 | CA | ILE | A | 99 | 10.875 | −101.039 | −26.311 | 1.00 | 38.53 | C |
| ATOM | 688 | CB | ILE | A | 99 | 11.319 | −100.724 | −27.756 | 1.00 | 39.11 | C |
| ATOM | 689 | CG1 | ILE | A | 99 | 11.736 | −102.021 | −28.467 | 1.00 | 38.03 | C |
| ATOM | 690 | CD1 | ILE | A | 99 | 12.222 | −101.807 | −29.890 | 1.00 | 40.05 | C |
| ATOM | 691 | CG2 | ILE | A | 99 | 12.431 | −99.660 | −27.794 | 1.00 | 38.22 | C |
| ATOM | 692 | C | ILE | A | 99 | 10.646 | −99.745 | −25.528 | 1.00 | 39.33 | C |
| ATOM | 693 | O | ILE | A | 99 | 11.519 | −99.266 | −24.787 | 1.00 | 36.71 | O |
| ATOM | 694 | N | ARG | A | 100 | 9.462 | −99.175 | −25.715 | 1.00 | 41.43 | N |
| ATOM | 695 | CA | ARG | A | 100 | 9.040 | −98.017 | −24.956 | 1.00 | 42.09 | C |
| ATOM | 696 | CB | ARG | A | 100 | 7.658 | −97.608 | −25.439 | 1.00 | 48.15 | C |
| ATOM | 697 | CG | ARG | A | 100 | 7.059 | −96.371 | −24.812 | 1.00 | 51.56 | C |
| ATOM | 698 | CD | ARG | A | 100 | 5.982 | −95.818 | −25.743 | 1.00 | 61.56 | C |
| ATOM | 699 | NE | ARG | A | 100 | 4.813 | −96.699 | −25.920 | 1.00 | 58.61 | N |
| ATOM | 700 | CZ | ARG | A | 100 | 4.338 | −97.100 | −27.108 | 1.00 | 63.98 | C |
| ATOM | 701 | NH1 | ARG | A | 100 | 4.915 | −96.716 | −28.243 | 1.00 | 53.50 | N |
| ATOM | 702 | NH2 | ARG | A | 100 | 3.264 | −97.888 | −27.167 | 1.00 | 65.09 | N |
| ATOM | 703 | C | ARG | A | 100 | 9.071 | −98.322 | −23.445 | 1.00 | 40.87 | C |
| ATOM | 704 | O | ARG | A | 100 | 9.643 | −97.558 | −22.687 | 1.00 | 39.83 | O |
| ATOM | 705 | N | ARG | A | 101 | 8.491 | −99.448 | −23.017 | 1.00 | 42.13 | N |
| ATOM | 706 | CA | ARG | A | 101 | 8.453 | −99.804 | −21.590 | 1.00 | 41.92 | C |
| ATOM | 707 | CB | ARG | A | 101 | 7.515 | −100.983 | −21.333 | 1.00 | 46.37 | C |
| ATOM | 708 | CG | ARG | A | 101 | 6.037 | −100.681 | −21.553 | 1.00 | 61.06 | C |
| ATOM | 709 | CD | ARG | A | 101 | 5.142 | −101.762 | −20.955 | 1.00 | 68.14 | C |
| ATOM | 710 | NE | ARG | A | 101 | 3.735 | −101.620 | −21.350 | 1.00 | 78.37 | N |
| ATOM | 711 | CZ | ARG | A | 101 | 2.836 | −100.872 | −20.708 | 1.00 | 85.80 | C |
| ATOM | 712 | NH1 | ARG | A | 101 | 3.182 | −100.179 | −19.630 | 1.00 | 97.56 | N |
| ATOM | 713 | NH2 | ARG | A | 101 | 1.583 | −100.813 | −21.139 | 1.00 | 81.70 | N |
| ATOM | 714 | C | ARG | A | 101 | 9.845 | −100.079 | −20.994 | 1.00 | 40.29 | C |
| ATOM | 715 | O | ARG | A | 101 | 10.193 | −99.547 | −19.931 | 1.00 | 39.67 | O |
| ATOM | 716 | N | ASN | A | 102 | 10.639 | −100.915 | −21.652 | 1.00 | 38.95 | N |
| ATOM | 717 | CA | ASN | A | 102 | 12.017 | −101.114 | −21.182 | 1.00 | 41.26 | C |
| ATOM | 718 | CB | ASN | A | 102 | 12.797 | −102.128 | −22.037 | 1.00 | 39.54 | C |
| ATOM | 719 | CG | ASN | A | 102 | 12.515 | −103.573 | −21.645 | 1.00 | 41.28 | C |
| ATOM | 720 | OD1 | ASN | A | 102 | 12.324 | −103.891 | −20.468 | 1.00 | 42.16 | O |
| ATOM | 721 | ND2 | ASN | A | 102 | 12.489 | −104.463 | −22.642 | 1.00 | 41.63 | N |
| ATOM | 722 | C | ASN | A | 102 | 12.758 | −99.791 | −21.135 | 1.00 | 37.16 | C |
| ATOM | 723 | O | ASN | A | 102 | 13.525 | −99.533 | −20.222 | 1.00 | 36.77 | O |
| ATOM | 724 | N | SER | A | 103 | 12.487 | −98.931 | −22.105 | 1.00 | 37.18 | N |
| ATOM | 725 | CA | SER | A | 103 | 13.192 | −97.668 | −22.157 | 1.00 | 35.70 | C |
| ATOM | 726 | CB | SER | A | 103 | 13.048 | −97.359 | −23.533 | 1.00 | 34.71 | C |
| ATOM | 727 | OG | SER | A | 103 | 13.812 | −97.803 | −24.473 | 1.00 | 31.90 | O |
| ATOM | 728 | C | SER | A | 103 | 12.803 | −96.723 | −20.995 | 1.00 | 36.30 | C |
| ATOM | 729 | O | SER | A | 103 | 13.681 | −96.128 | −20.357 | 1.00 | 33.48 | O |
| ATOM | 730 | N | GLU | A | 104 | 11.507 | −96.523 | −20.694 | 1.00 | 34.19 | N |
| ATOM | 731 | CA | GLU | A | 104 | 11.057 | −95.891 | −19.518 | 1.00 | 36.87 | C |
| ATOM | 732 | CB | GLU | A | 104 | 9.532 | −96.048 | −19.315 | 1.00 | 38.53 | C |
| ATOM | 733 | CG | GLU | A | 104 | 8.707 | −95.451 | −20.463 | 1.00 | 44.48 | C |
| ATOM | 734 | CD | GLU | A | 104 | 7.258 | −95.945 | −20.549 | 1.00 | 48.42 | C |
| ATOM | 735 | OE1 | GLU | A | 104 | 6.828 | −96.744 | −19.681 | 1.00 | 54.54 | O |
| ATOM | 736 | OE2 | GLU | A | 104 | 6.537 | −95.331 | −21.493 | 1.00 | 45.55 | O |
| ATOM | 737 | C | GLU | A | 104 | 11.832 | −96.385 | −18.289 | 1.00 | 35.25 | C |
| ATOM | 738 | O | GLU | A | 104 | 12.542 | −95.515 | −17.639 | 1.00 | 34.79 | O |
| ATOM | 739 | N | ALA | A | 105 | 11.712 | −97.575 | −17.994 | 1.00 | 34.67 | N |
| ATOM | 740 | CA | ALA | A | 105 | 12.370 | −98.271 | −16.825 | 1.00 | 35.31 | C |
| ATOM | 741 | CB | ALA | A | 105 | 12.033 | −99.741 | −16.724 | 1.00 | 33.70 | C |
| ATOM | 742 | C | ALA | A | 105 | 13.896 | −98.075 | −16.818 | 1.00 | 35.90 | C |
| ATOM | 743 | O | ALA | A | 105 | 14.494 | −97.797 | −15.757 | 1.00 | 37.05 | O |
| ATOM | 744 | N | ALA | A | 106 | 14.508 | −98.177 | −17.991 | 1.00 | 34.89 | N |
| ATOM | 745 | CA | ALA | A | 106 | 15.968 | −98.049 | −18.122 | 1.00 | 38.39 | C |
| ATOM | 746 | CB | ALA | A | 106 | 16.408 | −98.494 | −19.498 | 1.00 | 36.28 | C |
| ATOM | 747 | C | ALA | A | 106 | 16.424 | −91.627 | −17.855 | 1.00 | 38.51 | C |
| ATOM | 748 | O | ALA | A | 106 | 17.463 | −96.397 | −17.223 | 1.00 | 42.37 | O |
| ATOM | 749 | N | MET | A | 107 | 15.626 | −95.679 | −18.339 | 1.00 | 41.19 | N |
| ATOM | 750 | CA | MET | A | 107 | 15.883 | −94.255 | −18.180 | 1.00 | 39.68 | C |
| ATOM | 751 | CB | MET | A | 107 | 14.906 | −93.434 | −19.018 | 1.00 | 40.19 | C |
| ATOM | 752 | CG | MET | A | 107 | 15.084 | −91.938 | −18.879 | 1.00 | 35.92 | C |
| ATOM | 753 | SD | MET | A | 107 | 14.091 | −91.038 | −20.097 | 1.00 | 40.74 | S |
| ATOM | 754 | CE | MET | A | 107 | 14.389 | −89.351 | −19.582 | 1.00 | 30.33 | C |
| ATOM | 755 | C | MET | A | 107 | 15.766 | −93.884 | −16.711 | 1.00 | 39.51 | C |
| ATOM | 756 | O | MET | A | 107 | 16.668 | −93.265 | −16.164 | 1.00 | 42.04 | O |
| ATOM | 757 | N | LEU | A | 108 | 14.676 | −94.282 | −16.063 | 1.00 | 37.72 | N |
| ATOM | 758 | CA | LEU | A | 108 | 14.581 | −94.085 | −14.626 | 1.00 | 38.47 | C |
| ATOM | 759 | CB | LEU | A | 108 | 13.331 | −94.755 | −14.061 | 1.00 | 41.60 | C |
| ATOM | 760 | CG | LEU | A | 108 | 12.011 | −94.031 | −14.310 | 1.00 | 48.35 | C |
| ATOM | 761 | CD1 | LEU | A | 108 | 10.871 | −94.778 | −13.637 | 1.00 | 48.81 | C |
| ATOM | 762 | CD2 | LEU | A | 108 | 12.094 | −92.581 | −13.820 | 1.00 | 50.82 | C |
| ATOM | 763 | C | LEU | A | 108 | 15.829 | −94.627 | −13.921 | 1.00 | 37.54 | C |
| ATOM | 764 | O | LEU | A | 108 | 16.444 | −93.932 | −13.092 | 1.00 | 36.14 | O |
| ATOM | 765 | N | GLN | A | 109 | 16.180 | −95.871 | −14.247 | 1.00 | 36.85 | N |
| ATOM | 766 | CA | GLN | A | 109 | 17.292 | −96.548 | −13.617 | 1.00 | 34.97 | C |
| ATOM | 767 | CB | GLN | A | 109 | 17.431 | −97.461 | −14.152 | 1.00 | 38.58 | C |
| ATOM | 768 | CG | GLN | A | 109 | 18.616 | −98.193 | −13.601 | 1.00 | 36.97 | C |
| ATOM | 769 | CD | GLN | A | 109 | 18.767 | −100.083 | −14.385 | 1.00 | 36.96 | C |
| ATOM | 770 | OE1 | GLN | A | 109 | 18.719 | −100.072 | −15.626 | 1.00 | 39.41 | O |
| ATOM | 771 | NE2 | GLN | A | 109 | 18.903 | −101.200 | −13.687 | 1.00 | 36.66 | N |
| ATOM | 772 | C | GLN | A | 109 | 18.570 | −95.254 | −13.814 | 1.00 | 34.43 | C |
| ATOM | 773 | O | GLN | A | 109 | 19.252 | −95.459 | −12.843 | 1.00 | 33.78 | O |
| ATOM | 774 | N | GLU | A | 110 | 18.875 | −95.336 | −15.039 | 1.00 | 33.35 | N |
| ATOM | 775 | CA | GLU | A | 110 | 20.137 | −94.624 | −15.194 | 1.00 | 37.62 | C |
| ATOM | 776 | CB | GLU | A | 110 | 20.603 | −94.539 | −16.653 | 1.00 | 40.85 | C |
| ATOM | 777 | CG | GLU | A | 110 | 20.729 | −95.901 | −17.297 | 1.00 | 37.13 | C |
| ATOM | 778 | CD | GLU | A | 110 | 21.377 | −95.862 | −18.651 | 1.00 | 37.10 | C |
| ATOM | 779 | CE1 | GLU | A | 110 | 21.675 | −96.964 | −19.142 | 1.00 | 37.48 | O |
| ATOM | 780 | CE2 | GLU | A | 110 | 21.563 | −94.769 | −19.233 | 1.00 | 34.74 | O |
| ATOM | 781 | C | GLU | A | 110 | 20.071 | −93.266 | −14.539 | 1.00 | 39.10 | C |
| ATOM | 782 | O | GLU | A | 110 | 21.065 | −92.816 | −13.951 | 1.00 | 43.09 | O |
| ATOM | 783 | N | LEU | A | 111 | 18.902 | −92.626 | −14.618 | 1.00 | 25.93 | N |
| ATOM | 784 | CA | LEU | A | 111 | 18.697 | −91.352 | −13.934 | 1.00 | 24.32 | C |
| ATOM | 785 | CB | LEU | A | 111 | 17.356 | −90.711 | −14.333 | 1.00 | 31.33 | C |
| ATOM | 786 | CG | LEU | A | 111 | 17.371 | −89.994 | −15.698 | 1.00 | 30.05 | C |
| ATOM | 787 | CD1 | LEU | A | 111 | 15.963 | −89.558 | −16.042 | 1.00 | 28.25 | C |
| ATOM | 788 | CD2 | LEU | A | 111 | 18.317 | −88.794 | −15.746 | 1.00 | 26.52 | C |
| ATOM | 789 | C | LEU | A | 111 | 18.865 | −91.469 | −12.406 | 1.00 | 34.25 | C |
| ATOM | 790 | O | LEU | A | 111 | 19.626 | −90.719 | −11.792 | 1.00 | 37.18 | O |
| ATOM | 791 | N | ASN | A | 112 | 18.183 | −92.429 | −11.804 | 1.00 | 35.40 | N |
| ATOM | 792 | CA | ASN | A | 112 | 18.372 | −92.714 | −10.388 | 1.00 | 36.06 | C |
| ATOM | 793 | CB | ASN | A | 112 | 17.444 | −93.835 | −9.952 | 1.00 | 33.87 | C |
| ATOM | 794 | CG | ASN | A | 112 | 15.986 | −93.402 | −9.951 | 1.00 | 33.88 | C |
| ATOM | 795 | OD1 | ASN | A | 112 | 15.689 | −92.216 | −9.828 | 1.00 | 31.32 | O |
| ATOM | 796 | ND2 | ASN | A | 112 | 15.067 | −94.365 | −10.088 | 1.00 | 34.56 | N |
| ATOM | 797 | C | ASN | A | 112 | 19.820 | −93.002 | −9.995 | 1.00 | 40.07 | C |
| ATOM | 798 | O | ASN | A | 112 | 20.274 | −92.543 | −8.943 | 1.00 | 45.80 | O |
| ATOM | 799 | N | PHE | A | 113 | 20.562 | −93.714 | −10.843 | 1.00 | 37.61 | N |
| ATOM | 800 | CA | PHE | A | 113 | 21.977 | −93.979 | −10.553 | 1.00 | 37.15 | C |
| ATOM | 801 | CB | PHE | A | 113 | 22.556 | −95.020 | −11.530 | 1.00 | 33.97 | C |
| ATOM | 802 | CG | PHE | A | 113 | 23.693 | −95.780 | −10.959 | 1.00 | 37.61 | C |
| ATOM | 803 | CD1 | PHE | A | 113 | 23.493 | −96.640 | −9.882 | 1.00 | 35.43 | C |
| ATOM | 804 | CE1 | PHE | A | 113 | 24.553 | −97.337 | −9.331 | 1.00 | 38.01 | C |
| ATOM | 805 | CZ | PHE | A | 113 | 25.832 | −97.184 | −9.846 | 1.00 | 36.29 | C |
| ATOM | 806 | CE2 | PHE | A | 113 | 26.046 | −96.326 | −10.914 | 1.00 | 37.24 | C |
| ATOM | 807 | CD2 | PHE | A | 113 | 24.991 | −95.622 | −11.465 | 1.00 | 38.20 | C |
| ATOM | 808 | C | PHE | A | 113 | 22.818 | −92.707 | −10.560 | 1.00 | 37.25 | C |
| ATOM | 809 | O | PHE | A | 113 | 23.721 | −92.521 | −9.742 | 1.00 | 40.44 | O |
| ATOM | 810 | N | GLY | A | 114 | 22.329 | −91.808 | −11.460 | 1.00 | 36.63 | N |
| ATOM | 811 | CA | GLY | A | 114 | 23.302 | −90.574 | −11.545 | 1.00 | 37.05 | C |
| ATOM | 812 | C | GLY | A | 114 | 23.113 | −89.796 | −10.262 | 1.00 | 41.70 | C |
| ATOM | 813 | O | GLY | A | 114 | 24.085 | −89.274 | −9.678 | 1.00 | 46.66 | O |
| ATOM | 814 | N | ALA | A | 115 | 21.856 | −89.729 | −9.822 | 1.00 | 36.97 | N |
| ATOM | 815 | CA | ALA | A | 115 | 21.498 | −89.038 | −8.589 | 1.00 | 35.44 | C |
| ATOM | 816 | CB | ALA | A | 115 | 19.976 | −88.918 | −8.446 | 1.00 | 29.93 | C |
| ATOM | 817 | C | ALA | A | 115 | 22.107 | −89.746 | −7.383 | 1.00 | 34.76 | C |
| ATOM | 818 | O | ALA | A | 115 | 22.601 | −89.090 | −6.463 | 1.00 | 36.55 | O |
| ATOM | 819 | N | TYR | A | 116 | 22.061 | −91.079 | −7.391 | 1.00 | 35.10 | N |
| ATOM | 820 | CA | TYR | A | 116 | 22.714 | −91.876 | −6.355 | 1.00 | 35.19 | C |
| ATOM | 821 | CB | TYR | A | 116 | 22.530 | −93.362 | −6.636 | 1.00 | 35.22 | C |
| ATOM | 822 | CG | TYR | A | 116 | 23.378 | −94.287 | −5.766 | 1.00 | 39.65 | C |
| ATOM | 823 | CD1 | TYR | A | 116 | 22.963 | −94.662 | −4.486 | 1.00 | 38.06 | C |
| ATOM | 824 | CE1 | TYR | A | 116 | 23.725 | −95.515 | −3.709 | 1.00 | 33.94 | C |
| ATOM | 825 | CZ | TYR | A | 116 | 24.926 | −95.987 | −4.205 | 1.00 | 42.31 | C |
| ATOM | 826 | OH | TYR | A | 116 | 25.711 | −96.837 | −3.458 | 1.00 | 42.94 | O |
| ATOM | 827 | CE2 | TYR | A | 116 | 25.355 | −95.635 | −5.473 | 1.00 | 40.05 | C |
| ATOM | 828 | CD2 | TYR | A | 116 | 24.586 | −94.797 | −6.241 | 1.00 | 33.49 | C |
| ATOM | 829 | C | TYR | A | 116 | 24.204 | −91.488 | −6.246 | 1.00 | 35.23 | C |
| ATOM | 830 | O | TYR | A | 116 | 24.719 | −91.303 | −5.138 | 1.00 | 31.76 | O |
| ATOM | 831 | N | LEU | A | 117 | 24.853 | −91.325 | −7.402 | 1.00 | 35.21 | N |
| ATOM | 832 | CA | LEU | A | 117 | 26.280 | −90.976 | −7.490 | 1.00 | 35.40 | C |
| ATOM | 833 | CB | LEU | A | 117 | 26.849 | −91.284 | −8.882 | 1.00 | 32.32 | C |
| ATOM | 834 | CG | LEU | A | 117 | 26.848 | −92.754 | −9.256 | 1.00 | 30.92 | C |
| ATOM | 835 | CD1 | LEU | A | 117 | 27.464 | −92.960 | −10.627 | 1.00 | 33.31 | C |
| ATOM | 836 | CD2 | LEU | A | 117 | 27.577 | −93.546 | −8.191 | 1.00 | 25.52 | C |
| ATOM | 837 | C | LEU | A | 117 | 26.560 | −89.537 | −7.178 | 1.00 | 37.59 | C |
| ATOM | 838 | O | LEU | A | 117 | 27.648 | −89.213 | −6.757 | 1.00 | 39.30 | O |
| ATOM | 839 | N | GLY | A | 118 | 25.582 | −88.667 | −7.413 | 1.00 | 40.45 | N |
| ATOM | 840 | CA | GLY | A | 118 | 25.744 | −87.255 | −7.117 | 1.00 | 35.80 | C |
| ATOM | 841 | C | GLY | A | 118 | 26.335 | −86.527 | −8.300 | 1.00 | 36.27 | C |
| ATOM | 842 | O | GLY | A | 118 | 27.003 | −85.515 | −8.127 | 1.00 | 36.75 | O |
| ATOM | 843 | N | LEU | A | 119 | 26.096 | −87.039 | −9.507 | 1.00 | 33.66 | N |
| ATOM | 844 | CA | LEU | A | 119 | 26.626 | −86.385 | −10.701 | 1.00 | 32.60 | C |
| ATOM | 845 | CB | LEU | A | 119 | 26.358 | −87.204 | −11.965 | 1.00 | 30.49 | C |
| ATOM | 846 | CG | LEU | A | 119 | 26.734 | −88.596 | −11.885 | 1.00 | 30.32 | C |
| ATOM | 847 | CD1 | LEU | A | 119 | 26.392 | −89.395 | −13.194 | 1.00 | 28.65 | C |
| ATOM | 848 | CD2 | LEU | A | 119 | 28.214 | −88.388 | −11.514 | 1.00 | 29.42 | C |
| ATOM | 849 | C | LEU | A | 119 | 26.046 | −84.181 | −10.839 | 1.00 | 35.76 | C |
| ATOM | 850 | O | LEU | A | 119 | 24.824 | −84.776 | −10.622 | 1.00 | 35.55 | O |
| ATOM | 851 | N | PRO | A | 120 | 26.920 | −84.112 | −11.172 | 1.00 | 33.39 | N |
| ATOM | 852 | CA | PRO | A | 120 | 26.567 | −82.614 | −11.393 | 1.00 | 33.93 | C |
| ATOM | 853 | CB | PRO | A | 120 | 27.933 | −81.928 | −11.553 | 1.00 | 33.89 | C |
| ATOM | 854 | CG | PRO | A | 120 | 28.863 | −83.023 | −11.985 | 1.00 | 33.95 | C |
| ATOM | 855 | CD | PRO | A | 120 | 28.372 | −84.252 | −11.290 | 1.00 | 32.42 | C |
| ATOM | 856 | C | PRO | A | 120 | 25.749 | −82.401 | −12.677 | 1.00 | 35.76 | C |
| ATOM | 857 | O | PRO | A | 120 | 25.017 | −81.418 | −12.796 | 1.00 | 37.60 | O |
| ATOM | 858 | N | ALA | A | 121 | 25.916 | −83.282 | −13.645 | 1.00 | 33.31 | N |
| ATOM | 859 | CA | ALA | A | 121 | 25.240 | −83.150 | −14.912 | 1.00 | 35.37 | C |
| ATOM | 860 | CB | ALA | A | 121 | 26.017 | −82.232 | −15.860 | 1.00 | 38.33 | C |
| ATOM | 861 | C | ALA | A | 121 | 25.068 | −84.112 | −15.523 | 1.00 | 34.51 | C |
| ATOM | 862 | O | ALA | A | 121 | 25.731 | −83.453 | −15.136 | 1.00 | 36.33 | O |
| ATOM | 863 | N | PHE | A | 122 | 24.164 | −84.802 | −16.479 | 1.00 | 35.43 | N |
| ATOM | 864 | CA | PHE | A | 122 | 23.788 | −85.161 | −17.095 | 1.00 | 35.32 | C |
| ATOM | 865 | CB | PHE | A | 122 | 22.734 | −86.151 | −16.252 | 1.00 | 36.39 | C |
| ATOM | 866 | CG | PHE | A | 122 | 22.555 | −88.010 | −16.588 | 1.00 | 43.19 | C |
| ATOM | 867 | CD1 | PHE | A | 122 | 21.815 | −88.404 | −17.717 | 1.00 | 44.17 | C |
| ATOM | 868 | CE1 | PHE | A | 122 | 21.640 | −89.742 | −18.023 | 1.00 | 42.80 | C |
| ATOM | 869 | CZ | PHE | A | 122 | 22.207 | −90.706 | −17.213 | 1.00 | 42.07 | C |
| ATOM | 870 | CE2 | PHE | A | 122 | 22.940 | −90.129 | −16.091 | 1.00 | 40.14 | C |
| ATOM | 871 | CD2 | PHE | A | 122 | 23.111 | −88.193 | −15.782 | 1.00 | 36.98 | C |
| ATOM | 872 | C | PHE | A | 122 | 23.237 | −85.499 | −18.478 | 1.00 | 34.50 | C |
| ATOM | 873 | O | PHE | A | 122 | 22.445 | −84.179 | −18.595 | 1.00 | 32.90 | O |
| ATOM | 874 | N | LEU | A | 123 | 23.699 | −86.192 | −19.522 | 1.00 | 35.02 | N |
| ATOM | 875 | CA | LEU | A | 123 | 23.379 | −85.840 | −20.909 | 1.00 | 32.83 | C |
| ATOM | 876 | CB | LEU | A | 123 | 24.617 | −85.598 | −21.783 | 1.00 | 33.61 | C |
| ATOM | 877 | CG | LEU | A | 123 | 25.698 | −84.893 | −21.868 | 1.00 | 36.64 | C |
| ATOM | 878 | CD1 | LEU | A | 123 | 27.108 | −85.438 | −22.093 | 1.00 | 35.98 | C |
| ATOM | 879 | CD2 | LEU | A | 123 | 25.412 | −83.510 | −22.978 | 1.00 | 31.11 | C |
| ATOM | 880 | C | LEU | A | 123 | 22.244 | −86.205 | −21.453 | 1.00 | 32.50 | C |
| ATOM | 881 | O | LEU | A | 123 | 22.153 | −87.897 | −21.157 | 1.00 | 33.57 | O |
| ATOM | 882 | N | LEU | A | 124 | 21.369 | −86.101 | −22.245 | 1.00 | 31.29 | N |
| ATOM | 883 | CA | LEU | A | 124 | 20.314 | −86.851 | −22.913 | 1.00 | 29.75 | C |
| ATOM | 884 | CB | LEU | A | 124 | 18.987 | −86.291 | −22.140 | 1.00 | 28.35 | C |
| ATOM | 885 | CG | LEU | A | 124 | 18.906 | −87.273 | −20.676 | 1.00 | 31.38 | C |
| ATOM | 886 | CD1 | LEU | A | 124 | 19.026 | −86.126 | −19.685 | 1.00 | 29.83 | C |
| ATOM | 887 | CD2 | LEU | A | 124 | 17.655 | −88.061 | −20.358 | 1.00 | 33.30 | C |
| ATOM | 888 | C | LEU | A | 124 | 20.155 | −86.222 | −24.294 | 1.00 | 29.96 | C |
| ATOM | 889 | O | LEU | A | 124 | 20.109 | −85.017 | −24.410 | 1.00 | 28.89 | O |
| ATOM | 890 | N | PRO | A | 125 | 20.080 | −87.047 | −25.354 | 1.00 | 29.16 | N |
| ATOM | 891 | CA | PRO | A | 125 | 19.907 | −86.509 | −26.706 | 1.00 | 28.40 | C |
| ATOM | 892 | CB | PRO | A | 125 | 20.169 | −87.744 | −27.579 | 1.00 | 26.84 | C |
| ATOM | 893 | CG | PRO | A | 125 | 19.652 | −88.878 | −26.749 | 1.00 | 27.84 | C |
| ATOM | 894 | CD | PRO | A | 125 | 19.987 | −88.521 | −25.322 | 1.00 | 26.34 | C |
| ATOM | 895 | C | PRO | A | 125 | 18.477 | −86.029 | −27.022 | 1.00 | 30.19 | C |
| ATOM | 896 | O | PRO | A | 125 | 17.519 | −86.647 | −26.570 | 1.00 | 30.76 | O |
| ATOM | 897 | N | LEU | A | 126 | 18.354 | −84.655 | −27.803 | 1.00 | 31.23 | N |
| ATOM | 898 | CA | LEU | A | 126 | 17.171 | −84.765 | −28.645 | 1.00 | 32.32 | C |
| ATOM | 899 | CB | LEU | A | 126 | 16.521 | −83.376 | −28.481 | 1.00 | 29.72 | C |
| ATOM | 900 | CG | LEU | A | 126 | 16.205 | −82.988 | −27.035 | 1.00 | 28.85 | C |
| ATOM | 901 | CD1 | LEU | A | 126 | 15.994 | −81.486 | −26.946 | 1.00 | 26.19 | C |
| ATOM | 902 | CD2 | LEU | A | 126 | 15.024 | −83.765 | −26.461 | 1.00 | 27.95 | C |
| ATOM | 903 | C | LEU | A | 126 | 17.627 | −84.956 | −30.067 | 1.00 | 30.78 | C |
| ATOM | 904 | O | LEU | A | 126 | 18.241 | −84.087 | −30.643 | 1.00 | 30.08 | O |
| ATOM | 905 | N | ASN | A | 127 | 17.335 | −86.122 | −30.617 | 1.00 | 32.97 | N |
| ATOM | 906 | CA | ASN | A | 127 | 17.872 | −86.509 | −31.909 | 1.00 | 35.77 | C |
| ATOM | 907 | CB | ASN | A | 127 | 18.851 | −87.697 | −31.749 | 1.00 | 33.52 | C |
| ATOM | 908 | CG | ASN | A | 127 | 18.160 | −88.947 | −31.242 | 1.00 | 16.85 | C |
| ATOM | 909 | CD1 | ASN | A | 127 | 16.949 | −89.071 | −31.364 | 1.00 | 42.69 | O |
| ATOM | 910 | ND2 | ASN | A | 127 | 18.915 | −89.881 | −30.683 | 1.00 | 40.00 | N |
| ATOM | 911 | C | ASN | A | 127 | 16.727 | −86.844 | −32.845 | 1.00 | 35.92 | C |
| ATOM | 912 | O | ASN | A | 127 | 16.946 | −87.363 | −33.929 | 1.00 | 40.32 | O |
| ATOM | 913 | N | GLN | A | 128 | 15.501 | −86.565 | −32.402 | 1.00 | 16.76 | N |
| ATOM | 914 | CA | GLN | A | 128 | 14.334 | −36.622 | −33.261 | 1.00 | 16.24 | C |
| ATOM | 915 | CB | GLN | A | 128 | 13.730 | −88.034 | −33.267 | 1.00 | 16.87 | C |
| ATOM | 916 | CG | GLN | A | 128 | 13.245 | −88.521 | −31.913 | 1.00 | 40.55 | C |
| ATOM | 917 | CD | GLN | A | 128 | 12.756 | −89.956 | −31.948 | 1.00 | 47.09 | C |
| ATOM | 918 | CE1 | GLN | A | 128 | 13.455 | −90.838 | −32.454 | 1.00 | 48.46 | O |
| ATOM | 919 | NE2 | GLN | A | 128 | 11.550 | −90.206 | −31.398 | 1.00 | 43.24 | N |
| ATOM | 920 | C | GLN | A | 128 | 13.304 | −85.566 | −32.840 | 1.00 | 38.33 | C |
| ATOM | 921 | O | GLN | A | 128 | 13.511 | −84.844 | −31.862 | 1.00 | 39.56 | O |
| ATOM | 922 | N | GLU | A | 129 | 12.202 | −85.487 | −33.590 | 1.00 | 38.72 | N |
| ATOM | 923 | CA | GLU | A | 129 | 11.124 | −84.513 | −33.356 | 1.00 | 41.08 | C |
| ATOM | 924 | CB | GLU | A | 129 | 10.210 | −84.371 | −34.586 | 1.00 | 43.26 | C |
| ATOM | 925 | CG | GLU | A | 129 | 9.498 | −83.022 | −34.650 | 1.00 | 46.90 | C |
| ATOM | 926 | CD | GLU | A | 129 | 8.126 | −83.072 | −35.303 | 1.00 | 48.80 | C |
| ATOM | 927 | CE1 | GLU | A | 129 | 7.961 | −83.637 | −36.409 | 1.00 | 49.26 | O |
| ATOM | 928 | CE2 | GLU | A | 129 | 7.192 | −82.511 | −34.696 | 1.00 | 54.15 | O |
| ATOM | 929 | C | GLU | A | 129 | 10.269 | −84.840 | −32.133 | 1.00 | 35.82 | C |
| ATOM | 930 | O | GLU | A | 129 | 10.032 | −83.994 | −31.300 | 1.00 | 37.74 | O |
| ATOM | 931 | N | ASP | A | 130 | 9.816 | −86.071 | −32.027 | 1.00 | 23.92 | N |
| ATOM | 932 | CA | ASP | A | 130 | 8.926 | −86.431 | −30.945 | 1.00 | 25.08 | C |
| ATOM | 933 | CB | ASP | A | 130 | 7.836 | −87.406 | −31.431 | 1.00 | 31.41 | C |
| ATOM | 934 | CG | ASP | A | 130 | 6.904 | −87.815 | −30.329 | 1.00 | 32.59 | C |
| ATOM | 935 | OD1 | ASP | A | 130 | 6.974 | −87.219 | −29.224 | 1.00 | 31.18 | O |
| ATOM | 936 | OD2 | ASP | A | 130 | 6.077 | −88.729 | −30.564 | 1.00 | 37.35 | O |
| ATOM | 937 | C | ASP | A | 130 | 9.637 | −86.991 | −29.711 | 1.00 | 33.10 | C |
| ATOM | 938 | O | ASP | A | 130 | 9.985 | −88.138 | −29.675 | 1.00 | 35.49 | O |
| ATOM | 939 | N | ASN | A | 131 | 9.765 | −86.183 | −28.672 | 1.00 | 34.41 | N |
| ATOM | 940 | CA | ASN | A | 131 | 10.343 | −86.637 | −27.417 | 1.00 | 31.97 | C |
| ATOM | 941 | CB | ASN | A | 131 | 11.647 | −85.891 | −27.184 | 1.00 | 33.06 | C |
| ATOM | 942 | CG | ASN | A | 131 | 12.628 | −86.195 | −28.272 | 1.00 | 32.62 | C |
| ATOM | 943 | OD1 | ASN | A | 131 | 12.907 | −87.359 | −28.521 | 1.00 | 33.30 | O |
| ATOM | 944 | ND2 | ASN | A | 131 | 13.079 | −85.177 | −28.986 | 1.00 | 33.18 | N |
| ATOM | 945 | C | ASN | A | 131 | 9.404 | −86.482 | −26.259 | 1.00 | 32.61 | C |
| ATOM | 946 | O | ASN | A | 131 | 9.815 | −86.300 | −25.118 | 1.00 | 33.22 | O |
| ATOM | 947 | N | THR | A | 132 | 8.119 | −86.603 | −26.562 | 1.00 | 36.47 | N |
| ATOM | 948 | CA | THR | A | 132 | 7.107 | −86.378 | −25.578 | 1.00 | 34.63 | C |
| ATOM | 949 | CB | THR | A | 132 | 5.703 | −86.537 | −26.381 | 1.00 | 32.60 | C |
| ATOM | 950 | OG1 | THR | A | 132 | 5.590 | −85.661 | −27.308 | 1.00 | 33.17 | O |
| ATOM | 951 | CG2 | THR | A | 132 | 4.671 | −86.133 | −25.191 | 1.00 | 32.72 | C |
| ATOM | 952 | C | THR | A | 132 | 7.381 | −87.276 | −24.379 | 1.00 | 35.27 | C |
| ATOM | 953 | O | THR | A | 132 | 7.471 | −86.793 | −23.251 | 1.00 | 41.46 | O |
| ATOM | 954 | N | ASN | A | 133 | 7.574 | −88.561 | −24.616 | 1.00 | 33.91 | N |
| ATOM | 955 | CA | ASN | A | 133 | 7.653 | −89.498 | −23.501 | 1.00 | 34.90 | C |
| ATOM | 956 | CB | ASN | A | 133 | 7.421 | −90.932 | −23.964 | 1.00 | 35.59 | C |
| ATOM | 957 | CG | ASN | A | 133 | 6.981 | −91.837 | −22.836 | 1.00 | 37.71 | C |
| ATOM | 958 | OD1 | ASN | A | 133 | 6.203 | −91.438 | −21.969 | 1.00 | 35.86 | O |
| ATOM | 959 | ND2 | ASN | A | 133 | 7.483 | −93.071 | −22.836 | 1.00 | 36.93 | N |
| ATOM | 960 | C | ASN | A | 133 | 8.950 | −89.382 | −22.713 | 1.00 | 34.80 | C |
| ATOM | 961 | O | ASN | A | 133 | 3.951 | −89.550 | −21.495 | 1.00 | 34.13 | O |
| ATOM | 962 | N | LEU | A | 134 | 10.043 | −89.070 | −23.413 | 1.00 | 36.26 | N |
| ATOM | 963 | CA | LEU | A | 134 | 11.333 | −88.777 | −22.769 | 1.00 | 34.56 | C |
| ATOM | 964 | CB | LEU | A | 134 | 12.402 | −88.402 | −23.814 | 1.00 | 34.33 | C |
| ATOM | 965 | CG | LEU | A | 134 | 13.815 | −88.077 | −23.319 | 1.00 | 35.03 | C |
| ATOM | 966 | CD1 | LEU | A | 134 | 14.673 | −89.315 | −23.354 | 1.00 | 38.14 | C |
| ATOM | 967 | CD2 | LEU | A | 134 | 14.484 | −87.033 | −24.202 | 1.00 | 40.29 | C |
| ATOM | 968 | C | LEU | A | 134 | 11.129 | −87.646 | −21.778 | 1.00 | 34.38 | C |
| ATOM | 969 | O | LEU | A | 134 | 11.523 | −87.750 | −20.619 | 1.00 | 32.84 | O |
| ATOM | 970 | N | ALA | A | 135 | 10.482 | −86.570 | −22.248 | 1.00 | 36.19 | N |
| ATOM | 971 | CA | ALA | A | 135 | 10.109 | −85.423 | −21.415 | 1.00 | 33.14 | C |
| ATOM | 972 | CB | ALA | A | 135 | 9.330 | −84.408 | −22.250 | 1.00 | 33.61 | C |
| ATOM | 673 | C | ALA | A | 135 | 9.295 | −85.834 | −20.200 | 1.00 | 32.42 | C |
| ATOM | 974 | O | ALA | A | 135 | 9.499 | −85.335 | −19.096 | 1.00 | 32.65 | O |
| ATOM | 975 | N | ARG | A | 136 | 8.371 | −86.762 | −20.397 | 1.00 | 31.37 | N |
| ATOM | 976 | CA | ARG | A | 136 | 7.461 | −87.105 | −19.337 | 1.00 | 32.12 | C |
| ATOM | 977 | CB | ARG | A | 136 | 6.321 | −87.973 | −19.891 | 1.00 | 35.17 | C |
| ATOM | 978 | CG | ARG | A | 136 | 5.238 | −88.298 | −18.894 | 1.00 | 39.35 | C |
| ATOM | 979 | CD | ARG | A | 136 | 5.282 | −89.741 | −18.477 | 1.00 | 47.91 | C |
| ATOM | 980 | NE | ARG | A | 136 | 5.053 | −89.837 | −17.039 | 1.00 | 54.51 | N |
| ATOM | 981 | CZ | ARG | A | 136 | 5.094 | −90.965 | −16.339 | 1.00 | 58.03 | C |
| ATOM | 982 | NH1 | ARG | A | 136 | 4.870 | −90.940 | −15.021 | 1.00 | 53.04 | N |
| ATOM | 983 | NH2 | ARG | A | 136 | 5.357 | −92.115 | −16.958 | 1.00 | 57.15 | N |
| ATOM | 984 | C | ARG | A | 136 | 8.255 | −87.817 | −18.281 | 1.00 | 31.06 | C |
| ATOM | 985 | O | ARG | A | 136 | 8.125 | −87.541 | −17.092 | 1.00 | 30.27 | O |
| ATOM | 986 | N | VAL | A | 137 | 9.103 | −88.738 | −18.723 | 1.00 | 32.57 | N |
| ATOM | 987 | CA | VAL | A | 137 | 9.937 | −89.491 | −17.793 | 1.00 | 31.22 | C |
| ATOM | 988 | CB | VAL | A | 137 | 10.650 | −90.570 | −18.491 | 1.00 | 31.94 | C |
| ATOM | 989 | CG1 | VAL | A | 137 | 11.596 | −91.396 | −17.535 | 1.00 | 33.62 | C |
| ATOM | 990 | CG2 | VAL | A | 137 | 9.610 | −91.567 | −18.998 | 1.00 | 28.88 | C |
| ATOM | 991 | C | VAL | A | 137 | 10.881 | −88.557 | −17.036 | 1.00 | 29.94 | C |
| ATOM | 992 | O | VAL | A | 137 | 10.998 | −88.554 | −15.826 | 1.00 | 30.96 | O |
| ATOM | 993 | N | LEU | A | 138 | 11.487 | −87.502 | −17.722 | 1.00 | 31.14 | N |
| ATOM | 994 | CA | LEU | A | 138 | 12.464 | −86.734 | −17.059 | 1.00 | 34.45 | C |
| ATOM | 995 | CB | LEU | A | 138 | 13.264 | −85.313 | −18.079 | 1.00 | 30.40 | C |
| ATOM | 996 | CG | LEU | A | 138 | 14.264 | −84.346 | −17.576 | 1.00 | 31.89 | C |
| ATOM | 997 | CD1 | LEU | A | 138 | 15.410 | −85.413 | −16.750 | 1.00 | 32.07 | C |
| ATOM | 998 | CD2 | LEU | A | 138 | 14.859 | −84.454 | −18.728 | 1.00 | 27.74 | C |
| ATOM | 999 | C | LEU | A | 138 | 11.716 | −85.538 | −16.052 | 1.00 | 39.04 | C |
| ATOM | 1000 | O | LEU | A | 138 | 12.099 | −85.751 | −14.867 | 1.00 | 41.43 | O |
| ATOM | 1001 | N | THR | A | 139 | 10.630 | −85.211 | −16.514 | 1.00 | 35.31 | N |
| ATOM | 1002 | CA | THR | A | 139 | 9.809 | −84.392 | −15.630 | 1.00 | 35.02 | C |
| ATOM | 1003 | CB | THR | A | 139 | 8.531 | −83.316 | −16.317 | 1.00 | 36.66 | C |
| ATOM | 1004 | OG1 | THR | A | 139 | 8.893 | −83.085 | −17.492 | 1.00 | 36.44 | O |
| ATOM | 1005 | CG2 | THR | A | 139 | 7.789 | −82.555 | −15.402 | 1.00 | 34.17 | C |
| ATOM | 1006 | C | THR | A | 139 | 9.456 | −85.210 | −14.398 | 1.00 | 33.05 | C |
| ATOM | 1007 | O | THR | A | 139 | 9.588 | −84.733 | −13.271 | 1.00 | 32.26 | O |
| ATOM | 1008 | N | ASN | A | 140 | 9.056 | −86.461 | −14.585 | 1.00 | 35.53 | N |
| ATOM | 1009 | CA | ASN | A | 140 | 8.786 | −87.269 | −13.406 | 1.00 | 35.44 | C |
| ATOM | 1010 | CB | ASN | A | 140 | 8.204 | −88.617 | −13.750 | 1.00 | 38.75 | C |
| ATOM | 1011 | CG | ASN | A | 140 | 6.684 | −88.576 | −13.796 | 1.00 | 48.19 | C |
| ATOM | 1012 | OD1 | ASN | A | 140 | 6.079 | −87.864 | −14.622 | 1.00 | 44.09 | O |
| ATOM | 1013 | ND2 | ASN | A | 140 | 6.052 | −89.313 | −12.881 | 1.00 | 53.51 | N |
| ATOM | 1014 | C | ASN | A | 140 | 9.966 | −87.179 | −12.487 | 1.00 | 36.31 | C |
| ATOM | 1015 | O | ASN | A | 140 | 9.800 | −87.159 | −11.289 | 1.00 | 35.85 | O |
| ATOM | 1016 | N | HIS | A | 141 | 11.160 | −87.662 | −13.044 | 1.00 | 35.48 | N |
| ATOM | 1017 | CA | HIS | A | 141 | 12.364 | −87.832 | −12.243 | 1.00 | 33.15 | C |
| ATOM | 1018 | CB | HIS | A | 141 | 13.568 | −88.199 | −13.127 | 1.00 | 34.49 | C |
| ATOM | 1019 | CG | HIS | A | 141 | 14.896 | −88.155 | −12.418 | 1.00 | 30.28 | C |
| ATOM | 1020 | ND1 | HIS | A | 141 | 15.279 | −89.099 | −11.486 | 1.00 | 28.92 | N |
| ATOM | 1021 | CE1 | HIS | A | 141 | 16.485 | −88.103 | −11.029 | 1.00 | 29.08 | C |
| ATOM | 1022 | NE2 | HIS | A | 141 | 16.909 | −87.115 | −11.645 | 1.00 | 29.85 | N |
| ATOM | 1023 | CD2 | HIS | A | 141 | 15.936 | −87.295 | −12.527 | 1.00 | 29.68 | C |
| ATOM | 1024 | C | HIS | A | 141 | 12.633 | −86.342 | −11.506 | 1.00 | 36.12 | C |
| ATOM | 1025 | O | HIS | A | 141 | 12.988 | −86.351 | −10.324 | 1.00 | 37.23 | O |
| ATOM | 1026 | N | ILE | A | 142 | 12.446 | −85.424 | −12.202 | 1.00 | 38.27 | N |
| ATOM | 1027 | CA | ILE | A | 142 | 12.703 | −84.125 | −11.609 | 1.00 | 38.51 | C |
| ATOM | 1028 | CB | ILE | A | 142 | 12.533 | −82.990 | −12.631 | 1.00 | 38.01 | C |
| ATOM | 1029 | CG1 | ILE | A | 142 | 13.750 | −82.965 | −13.581 | 1.00 | 36.45 | C |
| ATOM | 1030 | CD1 | ILE | A | 142 | 13.630 | −82.019 | −14.760 | 1.00 | 32.51 | C |
| ATOM | 1031 | CG2 | ILE | A | 142 | 12.374 | −81.637 | −11.926 | 1.00 | 34.48 | C |
| ATOM | 1032 | C | ILE | A | 142 | 11.861 | −83.887 | −10.359 | 1.00 | 43.42 | C |
| ATOM | 1033 | O | ILE | A | 142 | 12.368 | −83.345 | −9.380 | 1.00 | 42.39 | O |
| ATOM | 1034 | N | HIS | A | 143 | 10.596 | −84.328 | −10.385 | 1.00 | 47.75 | N |
| ATOM | 1035 | CA | HIS | A | 143 | 9.679 | −84.134 | −9.265 | 1.00 | 46.81 | C |
| ATOM | 1036 | CB | HIS | A | 143 | 8.282 | −83.827 | −9.782 | 1.00 | 49.13 | C |
| ATOM | 1037 | CG | HIS | A | 143 | 8.185 | −82.500 | −10.458 | 1.00 | 53.22 | C |
| ATOM | 1038 | ND1 | HIS | A | 143 | 8.245 | −82.357 | −11.827 | 1.00 | 54.18 | N |
| ATOM | 1039 | CE1 | HIS | A | 143 | 8.148 | −81.077 | −12.138 | 1.00 | 53.73 | C |
| ATOM | 1040 | NE2 | HIS | A | 143 | 8.047 | −80.384 | −11.019 | 1.00 | 53.45 | N |
| ATOM | 1041 | CD2 | HIS | A | 143 | 8.066 | −81.249 | −9.952 | 1.00 | 53.65 | C |
| ATOM | 1042 | C | HIS | A | 143 | 9.643 | −85.306 | −3.288 | 1.00 | 50.12 | C |
| ATOM | 1043 | O | HIS | A | 143 | 8.616 | −85.562 | −7.646 | 1.00 | 45.98 | O |
| ATOM | 1044 | N | THR | A | 144 | 10.766 | −86.009 | −8.166 | 1.00 | 29.73 | N |
| ATOM | 1045 | CA | THR | A | 144 | 10.864 | −87.128 | −7.253 | 1.00 | 53.11 | C |
| ATOM | 1046 | CB | THR | A | 144 | 10.937 | −88.492 | −7.996 | 1.00 | 52.87 | C |
| ATOM | 1047 | OG1 | THR | A | 144 | 9.943 | −88.552 | −9.035 | 1.00 | 49.54 | O |
| ATOM | 1048 | CG2 | THR | A | 144 | 10.700 | −89.664 | −7.078 | 1.00 | 50.95 | C |
| ATOM | 1049 | C | THR | A | 144 | 12.082 | −86.858 | −6.380 | 1.00 | 63.08 | C |
| ATOM | 1050 | O | THR | A | 144 | 13.167 | −87.398 | −6.617 | 1.00 | 64.74 | O |
| ATOM | 1051 | N | GLY | A | 145 | 11.882 | −85.996 | −5.379 | 1.00 | 72.97 | N |
| ATOM | 1052 | CA | GLY | A | 145 | 12.966 | −85.433 | −4.566 | 1.00 | 84.14 | C |
| ATOM | 1053 | C | GLY | A | 145 | 13.407 | −84.112 | −5.178 | 1.00 | 96.89 | C |
| ATOM | 1054 | O | GLY | A | 145 | 13.275 | −83.918 | −6.391 | 1.00 | 100.53 | O |
| ATOM | 1055 | N | HIS | A | 146 | 13.917 | −83.195 | −4.358 | 1.00 | 104.46 | N |
| ATOM | 1056 | CA | HIS | A | 146 | 14.493 | −81.942 | −4.879 | 1.00 | 109.05 | C |
| ATOM | 1057 | CB | HIS | A | 146 | 14.007 | −80.718 | −4.082 | 1.00 | 108.83 | C |
| ATOM | 1058 | CG | HIS | A | 146 | 12.800 | −80.048 | −4.673 | 1.00 | 128.53 | C |
| ATOM | 1059 | ND1 | HIS | A | 146 | 12.892 | −79.057 | −5.632 | 1.00 | 132.36 | N |
| ATOM | 1060 | CE1 | HIS | A | 146 | 11.677 | −78.660 | −5.969 | 1.00 | 130.89 | C |
| ATOM | 1061 | NE2 | HIS | A | 146 | 10.800 | −79.354 | −5.261 | 1.00 | 131.49 | N |
| ATOM | 1062 | CD2 | HIS | A | 146 | 11.476 | −60.225 | −4.443 | 1.00 | 126.49 | C |
| ATOM | 1063 | C | HIS | A | 146 | 16.024 | −82.001 | −4.921 | 1.00 | 100.91 | C |
| ATOM | 1064 | O | HIS | A | 146 | 16.669 | −82.082 | −3.873 | 1.00 | 53.78 | O |
| ATOM | 1065 | N | HIS | A | 147 | 16.586 | −81.985 | −6.136 | 1.00 | 94.42 | N |
| ATOM | 1066 | CA | HIS | A | 147 | 18.049 | −82.034 | −6.350 | 1.00 | 87.06 | C |
| ATOM | 1067 | CB | HIS | A | 147 | 18.490 | −83.379 | −6.937 | 1.00 | 81.09 | C |
| ATOM | 1068 | CG | HIS | A | 147 | 17.940 | −84.553 | −6.194 | 1.00 | 94.04 | C |
| ATOM | 1069 | ND1 | HIS | A | 147 | 17.286 | −85.593 | −6.820 | 1.00 | 97.66 | N |
| ATOM | 1070 | CE1 | HIS | A | 147 | 16.888 | −86.468 | −5.914 | 1.00 | 59.03 | C |
| ATOM | 1071 | NE2 | HIS | A | 147 | 17.245 | −86.024 | −4.721 | 1.00 | 101.81 | N |
| ATOM | 1072 | CD2 | HIS | A | 147 | 17.902 | −84.827 | −4.868 | 1.00 | 93.88 | C |
| ATOM | 1073 | C | HIS | A | 147 | 18.548 | −80.883 | −7.212 | 1.00 | 81.98 | C |
| ATOM | 1074 | O | HIS | A | 147 | 17.766 | −80.251 | −7.928 | 1.00 | 74.40 | O |
| ATOM | 1075 | N | SER | A | 148 | 19.851 | −80.618 | −7.129 | 1.00 | 76.57 | N |
| ATOM | 1076 | CA | SER | A | 148 | 20.466 | −79.480 | −7.815 | 1.00 | 68.07 | C |
| ATOM | 1077 | CB | SER | A | 148 | 21.664 | −78.963 | −7.010 | 1.00 | 74.67 | C |
| ATOM | 1078 | OG | SER | A | 148 | 21.254 | −78.173 | −5.908 | 1.00 | 81.12 | O |
| ATOM | 1079 | C | SER | A | 148 | 20.923 | −79.796 | −9.243 | 1.00 | 59.76 | C |
| ATOM | 1080 | O | SER | A | 148 | 20.978 | −78.892 | −10.089 | 1.00 | 56.45 | O |
| ATOM | 1081 | N | SER | A | 149 | 21.235 | −81.071 | −9.494 | 1.00 | 48.97 | N |
| ATOM | 1082 | CA | SER | A | 149 | 21.967 | −81.488 | −10.698 | 1.00 | 44.37 | C |
| ATOM | 1083 | CB | SER | A | 149 | 22.325 | −82.957 | −10.602 | 1.00 | 39.20 | C |
| ATOM | 1084 | OG | SER | A | 149 | 21.213 | −83.739 | −10.907 | 1.00 | 34.92 | O |
| ATOM | 1085 | C | SER | A | 149 | 21.286 | −81.203 | −12.052 | 1.00 | 42.40 | C |
| ATOM | 1086 | O | SER | A | 149 | 20.047 | −81.255 | −12.170 | 1.00 | 40.71 | O |
| ATOM | 1087 | N | MET | A | 150 | 22.111 | −80.934 | −13.061 | 1.00 | 36.49 | N |
| ATOM | 1088 | CA | MET | A | 150 | 21.633 | −80.491 | −14.360 | 1.00 | 38.72 | C |
| ATOM | 1089 | CB | MET | A | 150 | 22.585 | −79.471 | −14.969 | 1.00 | 40.47 | C |
| ATOM | 1090 | CG | MET | A | 150 | 22.826 | −78.236 | −14.126 | 1.00 | 42.93 | C |
| ATOM | 1091 | SD | MET | A | 150 | 21.369 | −77.215 | −13.932 | 1.00 | 47.53 | S |
| ATOM | 1092 | CE | MET | A | 150 | 20.913 | −76.862 | −15.618 | 1.00 | 43.98 | C |
| ATOM | 1093 | C | MET | A | 150 | 21.440 | −81.598 | −15.374 | 1.00 | 39.06 | C |
| ATOM | 1094 | O | MET | A | 150 | 22.200 | −82.577 | −15.423 | 1.00 | 37.85 | O |
| ATOM | 1095 | N | PHE | A | 151 | 20.433 | −81.400 | −16.214 | 1.00 | 36.04 | N |
| ATOM | 1096 | CA | PHE | A | 151 | 20.241 | −82.229 | −17.373 | 1.00 | 34.87 | C |
| ATOM | 1097 | CB | PHE | A | 151 | 18.834 | −82.798 | −17.360 | 1.00 | 36.05 | C |
| ATOM | 1098 | CG | PHE | A | 151 | 18.529 | −83.495 | −16.090 | 1.00 | 34.58 | C |
| ATOM | 1099 | CD1 | PHE | A | 151 | 19.099 | −84.738 | −15.828 | 1.00 | 32.72 | C |
| ATOM | 1100 | CE1 | PHE | A | 151 | 18.860 | −85.372 | −14.620 | 1.00 | 35.71 | C |
| ATOM | 1101 | CZ | PHE | A | 151 | 18.043 | −84.755 | −13.654 | 1.00 | 36.52 | C |
| ATOM | 1102 | CE2 | PHE | A | 151 | 17.498 | −83.500 | −13.894 | 1.00 | 33.64 | C |
| ATOM | 1103 | CD2 | PHE | A | 151 | 17.757 | −82.870 | −15.109 | 1.00 | 34.38 | C |
| ATOM | 1104 | C | PHE | A | 151 | 20.554 | −81.456 | −18.626 | 1.00 | 35.92 | C |
| ATOM | 1105 | O | PHE | A | 151 | 19.864 | −80.500 | −18.954 | 1.00 | 37.71 | O |
| ATOM | 1106 | N | TRP | A | 152 | 21.633 | −81.867 | −19.296 | 1.00 | 35.79 | N |
| ATOM | 1107 | CA | TRP | A | 152 | 22.049 | −81.296 | −20.562 | 1.00 | 36.32 | C |
| ATOM | 1108 | CB | TRP | A | 152 | 23.578 | −81.331 | −20.691 | 1.00 | 37.11 | C |
| ATOM | 1109 | CG | TRP | A | 152 | 24.314 | −80.461 | −19.682 | 1.00 | 38.50 | C |
| ATOM | 1110 | CD1 | TRP | A | 152 | 23.764 | −79.535 | −18.824 | 1.00 | 41.00 | C |
| ATOM | 1111 | NE1 | TRP | A | 152 | 24.734 | −78.963 | −18.045 | 1.00 | 41.59 | N |
| ATOM | 1112 | CE2 | TRP | A | 152 | 25.946 | −79.484 | −18.403 | 1.00 | 40.77 | C |
| ATOM | 1113 | CD2 | TRP | A | 152 | 25.718 | −80.436 | −19.432 | 1.00 | 41.44 | C |
| ATOM | 1114 | CE3 | TRP | A | 152 | 26.809 | −81.131 | −19.964 | 1.00 | 41.97 | C |
| ATOM | 1115 | CZ3 | TRP | A | 152 | 28.083 | −80.348 | −19.465 | 1.00 | 43.70 | C |
| ATOM | 1116 | CH2 | TRP | A | 152 | 28.270 | −79.385 | −18.459 | 1.00 | 44.66 | C |
| ATOM | 1117 | CZ2 | TRP | A | 152 | 27.212 | −79.193 | −17.923 | 1.00 | 40.23 | C |
| ATOM | 1118 | C | TRP | A | 152 | 21.404 | −82.382 | −21.707 | 1.00 | 41.05 | C |
| ATOM | 1119 | O | TRP | A | 152 | 21.629 | −83.303 | −21.859 | 1.00 | 44.03 | O |
| ATOM | 1120 | N | MET | A | 153 | 20.581 | −81.388 | −22.491 | 1.00 | 37.18 | N |
| ATOM | 1121 | CA | MET | A | 153 | 20.023 | −81.960 | −23.700 | 1.00 | 36.25 | C |
| ATOM | 1122 | CB | MET | A | 153 | 18.638 | −81.387 | −24.020 | 1.00 | 36.44 | C |
| ATOM | 1123 | CG | MET | A | 153 | 17.721 | −81.328 | −22.817 | 1.00 | 32.67 | C |
| ATOM | 1124 | SD | MET | A | 153 | 17.508 | −82.370 | −21.899 | 1.00 | 34.26 | S |
| ATOM | 1125 | CE | MET | A | 153 | 16.956 | −83.940 | −23.218 | 1.00 | 31.18 | C |
| ATOM | 1126 | C | MET | A | 153 | 20.983 | −81.726 | −24.840 | 1.00 | 37.37 | C |
| ATOM | 1127 | O | MET | A | 153 | 21.430 | −80.504 | −25.108 | 1.00 | 38.14 | O |
| ATOM | 1128 | N | ARG | A | 154 | 21.328 | −82.311 | −25.505 | 1.00 | 36.44 | N |
| ATOM | 1129 | CA | ARG | A | 154 | 22.323 | −82.746 | −26.522 | 1.00 | 34.31 | C |
| ATOM | 1130 | CB | ARG | A | 154 | 23.166 | −84.036 | −26.487 | 1.00 | 36.42 | C |
| ATOM | 1131 | CG | ARG | A | 154 | 24.172 | −84.200 | −27.603 | 1.00 | 35.95 | C |
| ATOM | 1132 | CD | ARG | A | 154 | 25.175 | −85.272 | −27.190 | 1.00 | 45.99 | C |
| ATOM | 1133 | NE | ARG | A | 154 | 26.354 | −85.322 | −28.060 | 1.00 | 49.89 | N |
| ATOM | 1134 | CZ | ARG | A | 154 | 27.495 | −85.947 | −27.765 | 1.00 | 51.48 | C |
| ATOM | 1135 | NH1 | ARG | A | 154 | 27.643 | −86.592 | −26.608 | 1.00 | 46.22 | N |
| ATOM | 1136 | NH2 | ARG | A | 154 | 28.496 | −85.919 | −28.643 | 1.00 | 51.38 | N |
| ATOM | 1137 | C | ARG | A | 154 | 21.575 | −82.566 | −27.817 | 1.00 | 31.08 | C |
| ATOM | 1138 | O | ARG | A | 154 | 20.994 | −83.537 | −28.347 | 1.00 | 30.49 | O |
| ATOM | 1139 | N | VAL | A | 155 | 21.576 | −81.324 | −28.309 | 1.00 | 28.50 | N |
| ATOM | 1140 | CA | VAL | A | 155 | 20.893 | −80.996 | −29.567 | 1.00 | 30.78 | C |
| ATOM | 1141 | CB | VAL | A | 155 | 19.819 | −79.863 | −29.453 | 1.00 | 30.43 | C |
| ATOM | 1142 | CG1 | VAL | A | 155 | 18.622 | −80.203 | −30.329 | 1.00 | 31.14 | C |
| ATOM | 1143 | CG2 | VAL | A | 155 | 19.414 | −79.563 | −28.031 | 1.00 | 28.82 | C |
| ATOM | 1144 | C | VAL | A | 155 | 21.837 | −80.441 | −30.605 | 1.00 | 30.80 | C |
| ATOM | 1145 | O | VAL | A | 155 | 22.620 | −79.551 | −30.309 | 1.00 | 34.53 | O |
| ATOM | 1146 | N | PRO | A | 156 | 21.710 | −80.894 | −31.851 | 1.00 | 31.63 | N |
| ATOM | 1147 | CA | PRO | A | 156 | 22.595 | −80.319 | −32.877 | 1.00 | 32.38 | C |
| ATOM | 1148 | CB | PRO | A | 156 | 22.682 | −81.417 | −33.928 | 1.00 | 30.10 | C |
| ATOM | 1149 | CG | PRO | A | 156 | 21.489 | −82.278 | −33.711 | 1.00 | 31.72 | C |
| ATOM | 1150 | CD | PRO | A | 156 | 21.047 | −82.335 | −32.281 | 1.00 | 31.39 | C |
| ATOM | 1151 | C | PRO | A | 156 | 22.071 | −79.054 | −33.537 | 1.00 | 35.29 | C |
| ATOM | 1152 | O | PRO | A | 156 | 20.857 | −78.903 | −33.713 | 1.00 | 32.71 | O |
| ATOM | 1153 | N | LEU | A | 157 | 22.981 | −78.176 | −33.953 | 1.00 | 35.31 | N |
| ATOM | 1154 | CA | LEU | A | 157 | 22.585 | −77.067 | −34.807 | 1.00 | 39.56 | C |
| ATOM | 1155 | CB | LEU | A | 157 | 23.789 | −76.233 | −35.243 | 1.00 | 40.16 | C |
| ATOM | 1156 | CG | LEU | A | 157 | 24.280 | −75.244 | −34.192 | 1.00 | 43.04 | C |
| ATOM | 1157 | CD1 | LEU | A | 157 | 25.666 | −74.744 | −34.549 | 1.00 | 44.12 | C |
| ATOM | 1158 | CD2 | LEU | A | 157 | 23.289 | −74.093 | −34.024 | 1.00 | 43.87 | C |
| ATOM | 1159 | C | LEU | A | 157 | 21.842 | −77.584 | −36.024 | 1.00 | 40.93 | C |
| ATOM | 1160 | C | LEU | A | 157 | 20.792 | −77.060 | −36.377 | 1.00 | 50.14 | O |
| ATOM | 1161 | N | VAL | A | 158 | 22.386 | −78.620 | −36.656 | 1.00 | 42.85 | N |
| ATOM | 1162 | CA | VAL | A | 158 | 21.780 | −79.201 | −37.848 | 1.00 | 38.92 | C |
| ATOM | 1163 | CB | VAL | A | 158 | 22.716 | −79.134 | −39.092 | 1.00 | 37.97 | C |
| ATOM | 1164 | CG1 | VAL | A | 158 | 21.978 | −79.536 | −40.373 | 1.00 | 35.96 | C |
| ATOM | 1165 | CG2 | VAL | A | 158 | 23.298 | −77.742 | −39.263 | 1.00 | 35.66 | C |
| ATOM | 1166 | C | VAL | A | 158 | 21.396 | −80.629 | −37.545 | 1.00 | 38.08 | C |
| ATOM | 1167 | C | VAL | A | 158 | 22.196 | −81.385 | −37.023 | 1.00 | 45.83 | O |
| ATOM | 1168 | N | ALA | A | 159 | 20.156 | −80.974 | −37.864 | 1.00 | 38.56 | N |
| ATOM | 1169 | CA | ALA | A | 159 | 19.609 | −82.322 | −37.704 | 1.00 | 40.17 | C |
| ATOM | 1170 | CB | ALA | A | 159 | 18.122 | −82.333 | −38.094 | 1.00 | 36.87 | C |
| ATOM | 1171 | C | ALA | A | 159 | 20.372 | −83.303 | −38.570 | 1.00 | 45.97 | C |
| ATOM | 1172 | O | ALA | A | 159 | 20.705 | −82.985 | −39.711 | 1.00 | 52.42 | O |
| ATOM | 1173 | N | PRO | A | 160 | 20.645 | −84.507 | −33.044 | 1.00 | 50.70 | N |
| ATOM | 1174 | CA | PRO | A | 160 | 21.317 | −85.561 | −38.811 | 1.00 | 50.03 | C |
| ATOM | 1175 | CB | PRO | A | 160 | 21.085 | −86.818 | −37.956 | 1.00 | 54.44 | C |
| ATOM | 1176 | CG | PRO | A | 160 | 20.131 | −86.416 | −36.850 | 1.00 | 52.54 | C |
| ATOM | 1177 | CD | PRO | A | 160 | 20.327 | −84.948 | −36.675 | 1.00 | 51.21 | C |
| ATOM | 1178 | C | PRO | A | 160 | 20.769 | −85.738 | −40.247 | 1.00 | 51.06 | C |
| ATOM | 1179 | O | PRO | A | 160 | 21.553 | −85.752 | −41.197 | 1.00 | 53.29 | O |
| ATOM | 1180 | N | GLU | A | 161 | 19.442 | −85.828 | −40.388 | 1.00 | 49.78 | N |
| ATOM | 1181 | CA | GLU | A | 161 | 18.750 | −85.957 | −41.679 | 1.00 | 44.90 | C |
| ATOM | 1182 | CB | GLU | A | 161 | 17.237 | −86.021 | −41.452 | 1.00 | 47.87 | C |
| ATOM | 1183 | CG | GLU | A | 161 | 16.407 | −86.365 | −42.685 | 1.00 | 51.86 | C |
| ATOM | 1184 | CD | GLU | A | 161 | 16.845 | −87.658 | −43.359 | 1.00 | 55.37 | C |
| ATOM | 1185 | OE1 | GLU | A | 161 | 16.949 | −88.688 | −42.650 | 1.00 | 57.11 | O |
| ATOM | 1186 | OE2 | GLU | A | 161 | 17.073 | −87.643 | −44.595 | 1.00 | 54.32 | O |
| ATOM | 1187 | C | GLU | A | 161 | 19.046 | −84.862 | −42.691 | 1.00 | 43.77 | C |
| ATOM | 1188 | O | GLU | A | 161 | 16.589 | −85.075 | −43.893 | 1.00 | 44.32 | O |
| ATOM | 1189 | N | ASP | A | 162 | 19.462 | −83.693 | −42.208 | 1.00 | 42.40 | N |
| ATOM | 1190 | CA | ASP | A | 162 | 19.734 | −82.538 | −43.061 | 1.00 | 40.30 | C |
| ATOM | 1191 | CB | ASP | A | 162 | 19.077 | −81.263 | −42.475 | 1.00 | 58.33 | C |
| ATOM | 1192 | CG | ASP | A | 162 | 17.513 | −81.357 | −47.355 | 1.00 | 41.46 | C |
| ATOM | 1193 | OD1 | ASP | A | 162 | 16.816 | −82.021 | −43.167 | 1.00 | 39.09 | O |
| ATOM | 1194 | OD2 | ASP | A | 162 | 16.965 | −80.727 | −41.434 | 1.00 | 43.44 | O |
| ATOM | 1195 | C | ASP | A | 162 | 21.258 | −82.364 | −43.203 | 1.00 | 44.42 | C |
| ATOM | 1196 | O | ASP | A | 162 | 21.751 | −81.416 | −43.828 | 1.00 | 44.58 | O |
| ATOM | 1197 | N | LEU | A | 163 | 22.002 | −83.288 | −42.614 | 1.00 | 46.32 | N |
| ATOM | 1198 | CA | LEU | A | 163 | 23.467 | −83.248 | −42.675 | 1.00 | 52.90 | C |
| ATOM | 1199 | CB | LEU | A | 163 | 24.040 | −81.349 | −41.254 | 1.00 | 57.29 | C |
| ATOM | 1200 | CG | LEU | A | 163 | 25.246 | −82.509 | −40.838 | 1.00 | 59.96 | C |
| ATOM | 1201 | CD1 | LEU | A | 163 | 25.184 | −81.069 | −41.338 | 1.00 | 65.27 | C |
| ATOM | 1202 | CD2 | LEU | A | 163 | 25.339 | −82.537 | −39.324 | 1.00 | 62.99 | C |
| ATOM | 1203 | C | LEU | A | 163 | 24.020 | −84.385 | −43.554 | 1.00 | 49.20 | C |
| ATOM | 1204 | O | LEU | A | 163 | 25.182 | −84.358 | −43.944 | 1.00 | 49.60 | O |
| ATOM | 1205 | N | ARG | A | 164 | 23.177 | −85.374 | −43.860 | 1.00 | 46.43 | N |
| ATOM | 1206 | CA | ARG | A | 164 | 23.603 | −86.555 | −44.612 | 1.00 | 47.06 | C |
| ATOM | 1207 | CB | ARG | A | 164 | 22.480 | −87.601 | −44.670 | 1.00 | 44.90 | C |
| ATOM | 1208 | CG | ARG | A | 164 | 21.270 | −87.219 | −45.503 | 1.00 | 46.65 | C |
| ATOM | 1209 | CD | ARG | A | 164 | 20.257 | −88.357 | −45.570 | 1.00 | 50.19 | C |
| ATOM | 1210 | NE | ARG | A | 164 | 19.148 | −88.027 | −46.472 | 1.00 | 56.92 | N |
| ATOM | 1211 | CZ | ARG | A | 164 | 19.187 | −88.174 | −47.793 | 1.00 | 57.00 | C |
| ATOM | 1212 | NH1 | ARG | A | 164 | 20.274 | −88.659 | −48.386 | 1.00 | 61.67 | N |
| ATOM | 1213 | NH2 | ARG | A | 164 | 18.151 | −87.818 | −48.529 | 1.00 | 55.47 | N |
| ATOM | 1214 | C | ARG | A | 164 | 24.174 | −86.236 | −46.012 | 1.00 | 49.52 | C |
| ATOM | 1215 | O | ARG | A | 164 | 23.896 | −85.177 | −46.587 | 1.00 | 49.45 | O |
| ATOM | 1216 | N | ASP | A | 165 | 24.993 | −87.148 | −46.535 | 1.00 | 51.51 | N |
| ATOM | 1217 | CA | ASP | A | 165 | 25.573 | −87.006 | −47.876 | 1.00 | 52.52 | C |
| ATOM | 1218 | CB | ASP | A | 165 | 26.718 | −88.015 | −48.098 | 1.00 | 53.42 | C |
| ATOM | 1219 | CG | ASP | A | 165 | 27.919 | −87.771 | −47.184 | 1.00 | 56.05 | C |
| ATOM | 1220 | OD1 | ASP | A | 165 | 28.046 | −86.660 | −46.630 | 1.00 | 59.27 | O |
| ATOM | 1221 | OD2 | ASP | A | 165 | 28.745 | −88.693 | −47.008 | 1.00 | 61.29 | O |
| ATOM | 1222 | C | ASP | A | 165 | 24.487 | −87.218 | −48.921 | 1.00 | 52.66 | C |
| ATOM | 1223 | O | ASP | A | 165 | 23.546 | −88.007 | −48.719 | 1.00 | 54.79 | O |
| ATOM | 1224 | N | ASP | A | 166 | 24.611 | −86.516 | −50.038 | 1.00 | 51.34 | N |
| ATOM | 1225 | CA | ASP | A | 166 | 23.660 | −86.679 | −51.122 | 1.00 | 51.36 | C |
| ATOM | 1226 | CB | ASP | A | 166 | 23.655 | −85.432 | −51.988 | 1.00 | 52.28 | C |
| ATOM | 1227 | CG | ASP | A | 166 | 22.519 | −85.426 | −52.996 | 1.00 | 57.14 | C |
| ATOM | 1228 | OD1 | ASP | A | 166 | 21.562 | −86.249 | −52.885 | 1.00 | 52.61 | O |
| ATOM | 1229 | OD2 | ASP | A | 166 | 22.603 | −84.574 | −53.907 | 1.00 | 59.06 | O |
| ATOM | 1230 | C | ASP | A | 166 | 24.022 | −87.910 | −51.956 | 1.00 | 51.01 | C |
| ATOM | 1231 | O | ASP | A | 166 | 24.447 | −87.798 | −53.117 | 1.00 | 53.77 | O |
| ATOM | 1232 | N | ILE | A | 167 | 23.854 | −89.288 | −51.365 | 1.00 | 46.84 | N |
| ATOM | 1233 | CA | ILE | A | 167 | 24.388 | −90.302 | −51.971 | 1.00 | 43.18 | C |
| ATOM | 1234 | CB | ILE | A | 167 | 25.716 | −90.719 | −51.300 | 1.00 | 39.08 | C |
| ATOM | 1235 | CG1 | ILE | A | 167 | 25.528 | −90.997 | −49.802 | 1.00 | 39.74 | C |
| ATOM | 1236 | CD1 | ILE | A | 167 | 26.707 | −91.710 | −49.134 | 1.00 | 40.26 | C |
| ATOM | 1237 | CG2 | ILE | A | 167 | 26.736 | −89.600 | −51.454 | 1.00 | 39.31 | C |
| ATOM | 1238 | C | ILE | A | 167 | 23.345 | −91.426 | −52.067 | 1.00 | 44.32 | C |
| ATOM | 1239 | O | ILE | A | 167 | 23.680 | −92.601 | −52.266 | 1.00 | 44.50 | O |
| ATOM | 1240 | N | ILE | A | 168 | 22.074 | −91.362 | −51.958 | 1.00 | 43.76 | N |
| ATOM | 1241 | CA | ILE | A | 168 | 21.021 | −92.333 | −52.246 | 1.00 | 48.32 | C |
| ATOM | 1242 | CB | ILE | A | 168 | 19.966 | −92.140 | −51.110 | 1.00 | 44.72 | C |
| ATOM | 1243 | CG1 | ILE | A | 168 | 20.642 | −92.168 | −49.738 | 1.00 | 41.90 | C |
| ATOM | 1244 | CD1 | ILE | A | 168 | 19.735 | −92.536 | −48.589 | 1.00 | 41.83 | C |
| ATOM | 1245 | CG2 | ILE | A | 168 | 19.044 | −93.340 | −51.328 | 1.00 | 45.50 | C |
| ATOM | 1246 | C | ILE | A | 168 | 20.366 | −91.558 | −53.570 | 1.00 | 53.02 | C |
| ATOM | 1247 | O | ILE | A | 168 | 19.656 | −90.549 | −53.654 | 1.00 | 57.78 | O |
| ATOM | 1248 | N | AGLU | A | 169 | 20.635 | −92.461 | −54.599 | 0.50 | 53.43 | N |
| ATOM | 1249 | N | BGLU | A | 169 | 20.608 | −92.468 | −54.596 | 0.50 | 53.86 | N |
| ATOM | 1250 | CA | AGLU | A | 169 | 20.022 | −92.300 | −55.920 | 0.50 | 53.26 | C |
| ATOM | 1251 | CA | BGLU | A | 169 | 20.043 | −92.345 | −55.928 | 0.50 | 53.89 | C |
| ATOM | 1252 | CB | AGLU | A | 169 | 20.216 | −93.581 | −56.765 | 0.50 | 52.10 | C |
| ATOM | 1253 | CB | BGLU | A | 169 | 20.426 | −93.408 | −56.864 | 0.50 | 53.34 | C |
| ATOM | 1254 | CG | AGLU | A | 169 | 19.357 | −93.573 | −58.029 | 0.50 | 52.44 | C |
| ATOM | 1255 | CG | BGLU | A | 169 | 19.826 | −93.345 | −58.263 | 0.50 | 53.82 | C |
| ATOM | 1256 | CD | AGLU | A | 169 | 18.068 | −94.473 | −57.839 | 0.50 | 50.09 | C |
| ATOM | 1257 | CD | BGLU | A | 169 | 20.239 | −92.106 | −59.032 | 0.50 | 54.50 | C |
| ATOM | 1258 | OE1 | AGLU | A | 169 | 17.059 | −94.168 | −58.514 | 0.50 | 51.00 | O |
| ATOM | 1259 | OE1 | BGLU | A | 169 | 21.406 | −91.679 | −58.882 | 0.50 | 52.74 | O |
| ATOM | 1260 | OE2 | AGLU | A | 169 | 18.062 | −95.421 | −57.028 | 0.50 | 47.72 | O |
| ATOM | 1261 | OE2 | BGLU | A | 169 | 19.396 | −91.166 | −59.789 | 0.50 | 52.83 | O |
| ATOM | 1262 | C | AGLU | A | 169 | 18.534 | −91.920 | −55.813 | 0.50 | 53.83 | C |
| ATOM | 1263 | C | BGLU | A | 169 | 18.512 | −91.978 | −55.921 | 0.50 | 54.50 | C |
| ATOM | 1264 | O | AGLU | A | 169 | 18.139 | −90.139 | −56.245 | 0.50 | 55.06 | O |
| ATOM | 1265 | O | BGLU | A | 169 | 18.057 | −91.628 | −56.556 | 0.50 | 56.31 | O |
| ATOM | 1266 | N | ASN | A | 170 | 17.735 | −92.191 | −55.199 | 1.00 | 51.81 | N |
| ATOM | 1267 | CA | ASN | A | 170 | 16.260 | −97.615 | −55.139 | 1.00 | 57.08 | C |
| ATOM | 1268 | CB | ASN | A | 170 | 15.521 | −93.967 | −55.293 | 1.00 | 56.90 | C |
| ATOM | 1269 | CG | ASN | A | 170 | 15.864 | −94.973 | −54.190 | 1.00 | 57.51 | C |
| ATOM | 1270 | OD1 | ASN | A | 170 | 16.843 | −94.820 | −53.449 | 1.00 | 61.53 | O |
| ATOM | 1271 | ND2 | ASN | A | 170 | 15.066 | −96.030 | −54.102 | 1.00 | 54.48 | N |
| ATOM | 1272 | C | ASN | A | 170 | 15.687 | −91.849 | −53.928 | 1.00 | 58.96 | C |
| ATOM | 1273 | O | ASN | A | 170 | 14.493 | −91.943 | −53.640 | 1.00 | 60.16 | O |
| ATOM | 1274 | N | ALA | A | 171 | 16.539 | −91.110 | −53.222 | 1.00 | 58.15 | N |
| ATOM | 1275 | CA | ALA | A | 171 | 16.118 | −90.265 | −52.107 | 1.00 | 53.67 | C |
| ATOM | 1276 | CB | ALA | A | 171 | 16.149 | −91.042 | −50.798 | 1.00 | 48.28 | C |
| ATOM | 1277 | C | ALA | A | 171 | 17.068 | −89.077 | −52.040 | 1.00 | 58.40 | C |
| ATOM | 1278 | O | ALA | A | 171 | 17.899 | −88.997 | −51.130 | 1.00 | 55.58 | O |
| ATOM | 1279 | N | PRO | A | 172 | 16.975 | −88.159 | −53.021 | 1.00 | 62.35 | N |
| ATOM | 1280 | CA | PRO | A | 172 | 17.919 | −87.033 | −53.058 | 1.00 | 64.86 | C |
| ATOM | 1281 | CB | PRO | A | 172 | 17.569 | −86.325 | −54.374 | 1.00 | 66.53 | C |
| ATOM | 1282 | CG | PRO | A | 172 | 16.166 | −88.745 | −54.678 | 1.00 | 61.95 | C |
| ATOM | 1283 | CD | PRO | A | 172 | 16.061 | −88.154 | −54.180 | 1.00 | 60.92 | C |
| ATOM | 1284 | C | PRO | A | 172 | 17.738 | −81.071 | −51.884 | 1.00 | 68.16 | C |
| ATOM | 1285 | O | PRO | A | 172 | 16.637 | −85.964 | −51.332 | 1.00 | 68.80 | O |
| ATOM | 1286 | N | THR | A | 173 | 18.815 | −83.394 | −51500 | 1.00 | 54.36 | N |
| ATOM | 1287 | CA | THR | A | 173 | 18.727 | −84.379 | −50.471 | 1.00 | 69.34 | C |
| ATOM | 1288 | CB | THR | A | 173 | 20.102 | −84.063 | −49.831 | 1.00 | 65.31 | C |
| ATOM | 1289 | OG1 | THR | A | 173 | 21.031 | −85.643 | −50.840 | 1.00 | 56.92 | O |
| ATOM | 1290 | OG2 | THR | A | 173 | 20.656 | −85.269 | −49.082 | 1.00 | 87.76 | C |
| ATOM | 1291 | C | THR | A | 173 | 18.155 | −83.129 | −51.129 | 1.00 | 80.07 | C |
| ATOM | 1292 | O | THR | A | 173 | 18.585 | −82.752 | −52.225 | 1.00 | 86.71 | O |
| ATOM | 1293 | N | THR | A | 174 | 17.182 | −82.500 | −50.473 | 1.00 | 90.28 | N |
| ATOM | 1294 | CA | THR | A | 174 | 16.485 | −81.340 | −51.050 | 1.00 | 102.38 | C |
| ATOM | 1295 | CB | THR | A | 174 | 15.071 | −81.145 | −50.448 | 1.00 | 98.17 | C |
| ATOM | 1296 | CG1 | THR | A | 174 | 15.178 | −80.756 | −49.075 | 1.00 | 90.61 | O |
| ATOM | 1297 | CG2 | THR | A | 174 | 14.237 | −82.431 | −50.554 | 1.00 | 96.86 | C |
| ATOM | 1298 | C | THR | A | 174 | 17.315 | −86.050 | −50.925 | 1.00 | 111.09 | C |
| ATOM | 1299 | O | THR | A | 174 | 17.556 | −75.559 | −49.819 | 1.00 | 110.36 | C |
| ATOM | 1300 | N | HIS | A | 175 | 17.746 | −79.516 | −52.068 | 1.00 | 119.93 | N |
| ATOM | 1301 | CA | HIS | A | 175 | 18.618 | −78.332 | −52.113 | 1.00 | 129.39 | C |
| ATOM | 1302 | CB | HIS | A | 175 | 19.608 | −78.437 | −53.287 | 1.00 | 138.13 | C |
| ATOM | 1303 | CG | HIS | A | 175 | 20.919 | −79.075 | −52.931 | 1.00 | 140.99 | C |
| ATOM | 1304 | ND1 | HIS | A | 175 | 21.977 | −78.360 | −52.409 | 1.00 | 139.28 | N |
| ATOM | 1305 | CE1 | HIS | A | 175 | 22.997 | −79.174 | −52.203 | 1.00 | 137.94 | C |
| ATOM | 1306 | NE2 | HIS | A | 175 | 22.642 | −80.391 | −52.579 | 1.00 | 139.69 | N |
| ATOM | 1307 | CD2 | HIS | A | 175 | 21.348 | −80.355 | −53.042 | 1.00 | 139.66 | C |
| ATOM | 1308 | C | HIS | A | 175 | 17.856 | −76.998 | −52.184 | 1.00 | 126.54 | C |
| ATOM | 1309 | C | HIS | A | 175 | 18.468 | −75.935 | −52.331 | 1.00 | 123.54 | O |
| ATOM | 1310 | N | THR | A | 176 | 16.529 | −77.060 | −52.076 | 1.00 | 127.98 | N |
| ATOM | 1311 | CA | THR | A | 176 | 15.675 | −75.864 | −52.141 | 1.00 | 126.26 | C |
| ATOM | 1312 | CB | THR | A | 176 | 14.221 | −76.216 | −52.559 | 1.00 | 128.58 | C |
| ATOM | 1313 | OG1 | THR | A | 176 | 13.750 | −77.337 | −51.805 | 1.00 | 105.04 | O |
| ATOM | 1314 | OG2 | THR | A | 176 | 14.154 | −76.504 | −54.041 | 1.00 | 118.87 | C |
| ATOM | 1315 | C | THR | A | 176 | 15.701 | −75.058 | −50.827 | 1.00 | 129.29 | C |
| ATOM | 1316 | O | THR | A | 176 | 16.321 | −75.483 | −49.846 | 1.00 | 124.65 | O |
| ATOM | 1317 | N | GLU | A | 177 | 15.040 | −73.896 | −50.823 | 1.00 | 133.73 | N |
| ATOM | 1318 | CA | GLU | A | 177 | 15.007 | −72.988 | −49.660 | 1.00 | 133.42 | C |
| ATOM | 1319 | CB | GLU | A | 177 | 14.721 | −71.541 | −50.118 | 1.00 | 137.54 | C |
| ATOM | 1320 | CG | GLU | A | 177 | 14.513 | −70.506 | −49.008 | 1.00 | 136.67 | C |
| ATOM | 1321 | CD | GLU | A | 177 | 15.798 | −70.042 | −48.325 | 1.00 | 132.62 | C |
| ATOM | 1322 | OE1 | GLU | A | 177 | 16.861 | −70.684 | −48.492 | 1.00 | 124.62 | O |
| ATOM | 1323 | OE2 | GLU | A | 177 | 15.741 | −69.022 | −47.603 | 1.00 | 126.25 | O |
| ATOM | 1324 | C | GLU | A | 177 | 14.021 | −73.433 | −48.564 | 1.00 | 127.32 | C |
| ATOM | 1325 | O | GLU | A | 177 | 12.829 | −73.627 | −48.825 | 1.00 | 118.23 | O |
| ATOM | 1326 | N | GLU | A | 178 | 14.539 | −73.582 | −47.342 | 1.00 | 123.33 | N |
| ATOM | 1327 | CA | GLU | A | 178 | 13.765 | −74.065 | −46.191 | 1.00 | 121.84 | C |
| ATOM | 1328 | CB | GLU | A | 178 | 14.125 | −75.525 | −45.903 | 1.00 | 122.30 | C |
| ATOM | 1329 | CG | GLU | A | 178 | 13.129 | −76.564 | −46.385 | 1.00 | 119.96 | C |
| ATOM | 1330 | CD | GLU | A | 178 | 13.336 | −77.911 | −45.706 | 1.00 | 121.55 | C |
| ATOM | 1331 | OE1 | GLU | A | 178 | 14.470 | −78.210 | −45.263 | 1.00 | 117.94 | O |
| ATOM | 1332 | OE2 | GLU | A | 178 | 12.358 | −78.676 | −45.607 | 1.00 | 120.33 | O |
| ATOM | 1333 | C | GLU | A | 178 | 13.998 | −73.236 | −44.915 | 1.00 | 116.26 | C |
| ATOM | 1334 | O | GLU | A | 178 | 14.613 | −73.729 | −43.961 | 1.00 | 114.21 | O |
| ATOM | 1335 | N | TYR | A | 179 | 13.501 | −71.998 | −44.888 | 1.00 | 108.06 | N |
| ATOM | 1336 | CA | TYR | A | 179 | 13.728 | −71.108 | −43.739 | 1.00 | 100.36 | C |
| ATOM | 1337 | CB | TYR | A | 179 | 13.041 | −69.738 | −43.920 | 1.00 | 99.00 | C |
| ATOM | 1338 | CG | TYR | A | 179 | 13.634 | −68.643 | −43.039 | 1.00 | 96.77 | C |
| ATOM | 1339 | CD1 | TYR | A | 179 | 14.751 | −67.903 | −43.459 | 1.00 | 54.93 | C |
| ATOM | 1340 | CE1 | TYR | A | 179 | 15.302 | −66.911 | −47.653 | 1.00 | 90.64 | C |
| ATOM | 1341 | CZ | TYR | A | 179 | 14.736 | −66.646 | −41.405 | 1.00 | 88.11 | C |
| ATOM | 1342 | CZ | TYR | A | 179 | 15.275 | −65.667 | −40.602 | 1.00 | 86.42 | O |
| ATOM | 1343 | CE2 | TYR | A | 179 | 13.636 | −67.366 | −40.965 | 1.00 | 87.46 | C |
| ATOM | 1344 | CD2 | TYR | A | 179 | 13.091 | −68.356 | −41.778 | 1.00 | 93.02 | C |
| ATOM | 1345 | C | TYR | A | 179 | 13.313 | −71.767 | −42.416 | 1.00 | 95.17 | C |
| ATOM | 1346 | O | TYR | A | 179 | 14.135 | −71.883 | −41.492 | 1.00 | 79.83 | O |
| ATOM | 1347 | N | SER | A | 180 | 12.054 | −72.216 | −42.356 | 1.00 | 93.83 | N |
| ATOM | 1348 | CA | SER | A | 180 | 11.497 | −72.927 | −41.189 | 1.00 | 87.76 | C |
| ATOM | 1349 | CB | SER | A | 180 | 9.990 | −73.136 | −41.343 | 1.00 | 84.68 | C |
| ATOM | 1350 | OG | SFR | A | 180 | 9.296 | −72.457 | −40.318 | 1.00 | 81.89 | O |
| ATOM | 1351 | C | SER | A | 180 | 12.167 | −74.267 | −40.869 | 1.00 | 83.76 | C |
| ATOM | 1352 | O | SER | A | 180 | 12.551 | −74.508 | −39.714 | 1.00 | 74.06 | O |
| ATOM | 1353 | N | GLY | A | 181 | 12.288 | −75.125 | −41.888 | 1.00 | 76.76 | N |
| ATOM | 1354 | CA | GLY | A | 181 | 12.900 | −76.448 | −41.760 | 1.00 | 71.72 | C |
| ATOM | 1355 | C | GLY | A | 181 | 14.288 | −76.473 | −41.139 | 1.00 | 73.14 | C |
| ATOM | 1356 | O | GLY | A | 181 | 14.584 | −77.346 | −40.322 | 1.00 | 77.95 | O |
| ATOM | 1357 | N | GLU | A | 182 | 15.139 | −75.519 | −41.517 | 1.00 | 69.57 | N |
| ATOM | 1358 | CA | GLU | A | 182 | 16.498 | −75.402 | −40.951 | 1.00 | 68.05 | C |
| ATOM | 1359 | CB | GLU | A | 182 | 17.355 | −74.469 | −41.807 | 1.00 | 74.05 | C |
| ATOM | 1360 | CG | GLU | A | 182 | 17.879 | −75.153 | −43.071 | 1.00 | 85.20 | C |
| ATOM | 1361 | CD | GLU | A | 182 | 17.861 | −74.262 | −44.306 | 1.00 | 85.66 | C |
| ATOM | 1362 | OE1 | GLU | A | 182 | 17.137 | −74.615 | −45.268 | 1.00 | 88.53 | O |
| ATOM | 1363 | OE2 | GLU | A | 182 | 18.564 | −73.222 | −44.322 | 1.00 | 80.17 | O |
| ATOM | 1364 | C | GLU | A | 182 | 16.504 | −74.957 | −39.488 | 1.00 | 62.09 | C |
| ATOM | 1365 | O | GLU | A | 182 | 17.544 | −74.972 | −38.832 | 1.00 | 59.86 | O |
| ATOM | 1366 | N | GLU | A | 183 | 15.332 | −74.574 | −38.984 | 1.00 | 52.21 | N |
| ATOM | 1367 | CA | GLU | A | 183 | 15.139 | −74.342 | −37.559 | 1.00 | 50.42 | C |
| ATOM | 1368 | CB | GLU | A | 183 | 14.337 | −73.073 | −37.350 | 1.00 | 55.10 | C |
| ATOM | 1369 | CG | GLU | A | 183 | 15.109 | −71.791 | −37.588 | 1.00 | 59.16 | C |
| ATOM | 1370 | CD | GLU | A | 183 | 14.174 | −70.632 | −37.807 | 1.00 | 55.54 | C |
| ATOM | 1371 | OE1 | GLU | A | 183 | 12.962 | −70.807 | −37.565 | 1.00 | 52.47 | O |
| ATOM | 1372 | OE2 | GLU | A | 183 | 14.654 | −69.561 | −38.213 | 1.00 | 61.07 | O |
| ATOM | 1373 | C | GLU | A | 183 | 14.420 | −75.498 | −36.835 | 1.00 | 44.62 | C |
| ATOM | 1374 | O | GLU | A | 183 | 13.977 | −79.337 | −35.693 | 1.00 | 42.86 | O |
| ATOM | 1375 | N | LYS | A | 184 | 14.310 | −76.657 | −37.478 | 1.00 | 38.64 | N |
| ATOM | 1376 | CA | LYS | A | 184 | 13.520 | −77.739 | −36.895 | 1.00 | 43.93 | O |
| ATOM | 1377 | CB | LYS | A | 184 | 13.478 | −78.969 | −37.813 | 1.00 | 43.61 | C |
| ATOM | 1378 | CG | LYS | A | 184 | 14.820 | −79.552 | −38.063 | 1.00 | 43.95 | C |
| ATOM | 1379 | CD | LYS | A | 184 | 14.623 | −81.033 | −38.698 | 1.00 | 46.05 | C |
| ATOM | 1380 | CE | LYS | A | 184 | 13.694 | −80.014 | −39.918 | 1.00 | 46.44 | C |
| ATOM | 1381 | NZ | LYS | A | 184 | 14.249 | −80.189 | −41.042 | 1.00 | 47.35 | N |
| ATOM | 1382 | C | LYS | A | 184 | 14.028 | −78.096 | −35.497 | 1.00 | 42.94 | C |
| ATOM | 1383 | O | LYS | A | 184 | 13.255 | −78.506 | −34.582 | 1.00 | 42.94 | O |
| ATOM | 1384 | N | THR | A | 185 | 15.340 | −77.986 | −38.363 | 1.00 | 38.28 | N |
| ATOM | 1385 | CA | THR | A | 185 | 16.082 | −78.421 | −34.218 | 1.00 | 37.56 | C |
| ATOM | 1386 | CB | THR | A | 185 | 17.579 | −78.471 | −34.615 | 1.00 | 37.72 | C |
| ATOM | 1387 | OG1 | THR | A | 185 | 17.959 | −79.344 | −34.778 | 1.00 | 45.90 | O |
| ATOM | 1388 | CG2 | THR | A | 185 | 18.449 | −77.340 | −33.634 | 1.00 | 32.21 | C |
| ATOM | 1389 | C | THR | A | 185 | 15.802 | −77.566 | −32.991 | 1.00 | 36.52 | C |
| ATOM | 1390 | O | THR | A | 185 | 15.644 | −78.084 | −31.868 | 1.00 | 36.07 | O |
| ATOM | 1391 | N | TRP | A | 186 | 15.745 | −76.259 | −33.194 | 1.00 | 34.91 | N |
| ATOM | 1392 | CA | TRP | A | 186 | 15.294 | −75.383 | −32.126 | 1.00 | 35.33 | C |
| ATOM | 1393 | CB | TRP | A | 186 | 15.352 | −73.907 | −32.544 | 1.00 | 34.80 | C |
| ATOM | 1394 | CG | TRP | A | 186 | 14.945 | −73.018 | −31.438 | 1.00 | 35.52 | C |
| ATOM | 1395 | CD1 | TRP | A | 186 | 15.738 | −72.506 | −30.462 | 1.00 | 38.07 | C |
| ATOM | 1396 | NE1 | TRP | A | 186 | 14.996 | −71.750 | −29.593 | 1.00 | 37.48 | N |
| ATOM | 1397 | CE2 | TRP | A | 186 | 13.689 | −71.777 | −29.995 | 1.00 | 38.70 | C |
| ATOM | 1398 | CD2 | TRP | A | 186 | 13.621 | −72.572 | −31.157 | 1.00 | 40.87 | C |
| ATOM | 1399 | CE3 | TRP | A | 186 | 12.368 | −72.781 | −31.774 | 1.00 | 38.85 | C |
| ATOM | 1400 | CZ3 | TRP | A | 186 | 11.267 | −72.198 | −31.224 | 1.00 | 41.00 | C |
| ATOM | 1401 | CH2 | TRP | A | 186 | 11.365 | −71.396 | −30.068 | 1.00 | 43.41 | C |
| ATOM | 1402 | CZ2 | TRP | A | 186 | 12.568 | −71.178 | −29.438 | 1.00 | 41.72 | C |
| ATOM | 1403 | C | TRP | A | 186 | 13.878 | −75.791 | −31.650 | 1.00 | 32.76 | C |
| ATOM | 1404 | O | TRP | A | 186 | 13.626 | −75.826 | −30.448 | 1.00 | 35.34 | O |
| ATOM | 1405 | N | AMET | A | 187 | 12.987 | −76.131 | −32.577 | 0.50 | 31.43 | N |
| ATOM | 1406 | N | BMET | A | 187 | 12.986 | −76.122 | −32.586 | 0.50 | 31.08 | N |
| ATOM | 1407 | CA | AMET | A | 187 | 11.635 | −76.802 | −32.178 | 0.50 | 32.20 | C |
| ATOM | 1408 | CA | BMET | A | 187 | 11.624 | −76.533 | −32.227 | 0.50 | 31.43 | C |
| ATOM | 1409 | CB | AMET | A | 187 | 10.686 | −76.583 | −33.371 | 0.50 | 31.98 | C |
| ATOM | 1410 | CB | BMET | A | 187 | 10.734 | −76.713 | −33.463 | 0.50 | 30.90 | C |
| ATOM | 1411 | CG | AMET | A | 187 | 9.225 | −76.645 | −32.962 | 0.50 | 34.13 | C |
| ATOM | 1412 | CG | BMET | A | 187 | 9.543 | −77.646 | −33.260 | 0.50 | 31.57 | C |
| ATOM | 1413 | SD | AMET | A | 187 | 8.671 | −75.127 | −32.152 | 0.50 | 35.19 | S |
| ATOM | 1414 | SD | BMET | A | 187 | 9.689 | −79.290 | −34.010 | 0.50 | 12.70 | S |
| ATOM | 1415 | CE | AMET | A | 187 | 8.771 | −73.964 | −33.515 | 0.50 | 29.86 | C |
| ATOM | 1416 | CE | BMET | A | 187 | 10.066 | −78.829 | −35.706 | 0.50 | 29.35 | C |
| ATOM | 1417 | C | AMET | A | 187 | 11.624 | −77.787 | −31.350 | 0.50 | 12.21 | C |
| ATOM | 1418 | C | BMET | A | 187 | 11.630 | −77.782 | −31.355 | 0.50 | 31.79 | C |
| ATOM | 1419 | C | AMET | A | 187 | 10.813 | −77.928 | −30.420 | 0.50 | 32.68 | O |
| ATOM | 1420 | O | BMET | A | 187 | 10.837 | −77.894 | −30.404 | 0.50 | 32.28 | O |
| ATOM | 1421 | N | TRP | A | 188 | 12.547 | −73.700 | −31.657 | 1.00 | 31.38 | N |
| ATOM | 1422 | CA | TRP | A | 188 | 12.712 | −70.935 | −30.870 | 1.00 | 30.97 | C |
| ATOM | 1423 | CB | TRP | A | 188 | 13.897 | −86.768 | −31.375 | 1.00 | 30.32 | C |
| ATOM | 1424 | CG | TRP | A | 188 | 13.779 | −81.367 | −32.738 | 1.00 | 33.37 | C |
| ATOM | 1425 | CD1 | TRP | A | 188 | 12.709 | −81.301 | −33.588 | 1.00 | 33.06 | C |
| ATOM | 1426 | NE1 | TRP | A | 188 | 12.986 | −81.987 | −34.749 | 1.00 | 35.59 | N |
| ATOM | 1427 | CE2 | TRP | A | 188 | 14.255 | −82.513 | −34.669 | 1.00 | 34.58 | C |
| ATOM | 1428 | CD2 | TRP | A | 188 | 14.786 | −82.145 | −33.411 | 1.00 | 33.60 | C |
| ATOM | 1429 | CE3 | TRP | A | 188 | 16.085 | −82.562 | −33.070 | 1.00 | 32.60 | C |
| ATOM | 1430 | CZ3 | TRP | A | 188 | 16.808 | −83.339 | −33.984 | 1.00 | 34.00 | C |
| ATOM | 1431 | CH2 | TRP | A | 188 | 16.249 | −83.700 | −35.234 | 1.00 | 34.57 | C |
| ATOM | 1432 | CZ2 | TRP | A | 188 | 14.980 | −81.299 | −15.597 | 1.00 | 33.65 | C |
| ATOM | 1433 | C | TRP | A | 188 | 12.954 | −79.612 | −29.396 | 1.00 | 30.98 | C |
| ATOM | 1434 | O | TRP | A | 188 | 12.310 | −80.176 | −28.472 | 1.00 | 34.18 | O |
| ATOM | 1435 | N | TRP | A | 189 | 13.888 | −78.690 | −29.197 | 1.00 | 27.49 | N |
| ATOM | 1436 | CA | TRP | A | 189 | 14.238 | −78.198 | −27.885 | 1.00 | 25.43 | C |
| ATOM | 1437 | CB | TRP | A | 189 | 15.509 | −77.365 | −27.995 | 1.00 | 21.27 | C |
| ATOM | 1438 | CG | TRP | A | 189 | 15.626 | −76.358 | −26.931 | 1.00 | 29.24 | C |
| ATOM | 1439 | CD1 | TRP | A | 189 | 15.546 | −74.994 | −27.081 | 1.00 | 28.17 | C |
| ATOM | 1440 | NE1 | TRP | A | 189 | 15.711 | −74.577 | −25.855 | 1.00 | 27.62 | N |
| ATOM | 1441 | CE2 | TRP | A | 189 | 15.926 | −75.336 | −24.905 | 1.00 | 27.74 | C |
| ATOM | 1442 | CD2 | TRP | A | 189 | 15.875 | −76.596 | −25.546 | 1.00 | 28.09 | C |
| ATOM | 1443 | CE3 | TRP | A | 189 | 16.082 | −77.746 | −24.790 | 1.00 | 20.51 | C |
| ATOM | 1444 | CZ3 | TRP | A | 189 | 16.308 | −77.613 | −23.416 | 1.00 | 21.33 | C |
| ATOM | 1445 | CH2 | TRP | A | 189 | 16.320 | −76.340 | −22.801 | 1.00 | 20.68 | C |
| ATOM | 1446 | CZ2 | TRP | A | 189 | 16.125 | −75.197 | −23.535 | 1.00 | 29.27 | C |
| ATOM | 1447 | C | TRP | A | 189 | 13.130 | −77.316 | −27.294 | 1.00 | 25.22 | C |
| ATOM | 1448 | C | TRP | A | 189 | 12.789 | −77.444 | −26.109 | 1.00 | 24.83 | O |
| ATOM | 1449 | N | HIS | A | 190 | 12.647 | −76.359 | −28.077 | 1.00 | 25.34 | N |
| ATOM | 1450 | CA | HIS | A | 190 | 11.520 | −75.541 | −27.588 | 1.00 | 29.33 | C |
| ATOM | 1451 | CB | HIS | A | 190 | 10.944 | −74.647 | −28.665 | 1.00 | 29.69 | C |
| ATOM | 1452 | CG | HIS | A | 190 | 9.806 | −73.799 | −28.186 | 1.00 | 34.05 | C |
| ATOM | 1453 | ND1 | HIS | A | 190 | 9.934 | −72.900 | −27.147 | 1.00 | 35.81 | N |
| ATOM | 1454 | CE1 | HIS | A | 190 | 8.787 | −72.289 | −26.946 | 1.00 | 30.34 | C |
| ATOM | 1455 | NE2 | HIS | A | 190 | 7.920 | −72.749 | −27.826 | 1.00 | 32.98 | N |
| ATOM | 1456 | CD2 | HIS | A | 190 | 8.525 | −73.705 | −28.604 | 1.00 | 29.25 | C |
| ATOM | 1457 | C | HIS | A | 190 | 10.438 | −76.444 | −27.011 | 1.00 | 25.83 | C |
| ATOM | 1458 | O | HIS | A | 190 | 10.120 | −76.329 | −25.840 | 1.00 | 24.66 | O |
| ATOM | 1459 | N | ASN | A | 191 | 9.959 | −77.397 | −27.802 | 1.00 | 27.12 | N |
| ATOM | 1460 | CA | ASN | A | 191 | 8.839 | −78.254 | −27.359 | 1.00 | 30.70 | C |
| ATOM | 1461 | CB | ASN | A | 191 | 8.376 | −79.187 | −28.468 | 1.00 | 29.58 | C |
| ATOM | 1462 | CG | ASN | A | 191 | 7.711 | −78.434 | −29.584 | 1.00 | 31.40 | C |
| ATOM | 1463 | OD1 | ASN | A | 191 | 7.492 | −77.233 | −29.478 | 1.00 | 34.13 | N |
| ATOM | 1464 | ND2 | ASN | A | 191 | 7.400 | −79.113 | −30.657 | 1.00 | 32.43 | N |
| ATOM | 1465 | C | ASN | A | 191 | 9.164 | −79.050 | −26.139 | 1.00 | 32.59 | C |
| ATOM | 1466 | O | ASN | A | 191 | 8.311 | −79.185 | −25.253 | 1.00 | 38.14 | O |
| ATOM | 1467 | N | PHE | A | 192 | 10.397 | −79.564 | −26.070 | 1.00 | 33.01 | N |
| ATOM | 1468 | CA | PHE | A | 192 | 10.769 | −80.461 | −21.976 | 1.00 | 31.17 | C |
| ATOM | 1469 | CB | PHE | A | 192 | 12.174 | −83.047 | −25.189 | 1.00 | 28.17 | C |
| ATOM | 1470 | CG | PHE | A | 192 | 12.651 | −81.922 | −24.055 | 1.00 | 26.09 | C |
| ATOM | 1471 | CD1 | PHE | A | 192 | 12.253 | −83.248 | −23.968 | 1.00 | 25.91 | C |
| ATOM | 1472 | CE1 | PHE | A | 192 | 12.676 | −84.068 | −22.926 | 1.00 | 24.69 | C |
| ATOM | 1473 | CZ | PHE | A | 192 | 13.545 | −83.557 | −21.971 | 1.00 | 29.24 | C |
| ATOM | 1474 | CE2 | PHE | A | 192 | 13.977 | −82.234 | −22.050 | 1.00 | 27.98 | C |
| ATOM | 1475 | CD2 | PHE | A | 192 | 13.522 | −81.422 | −23.087 | 1.00 | 27.76 | C |
| ATOM | 1476 | C | PHE | A | 192 | 10.722 | −79.685 | −23.683 | 1.00 | 32.70 | C |
| ATOM | 1477 | O | PHE | A | 192 | 10.188 | −80.149 | −22.657 | 1.00 | 36.27 | O |
| ATOM | 1478 | N | ARG | A | 193 | 11.274 | −78.488 | −73.756 | 1.00 | 30.75 | N |
| ATOM | 1479 | CA | ARG | A | 193 | 11.442 | −77.649 | −22.605 | 1.00 | 34.11 | C |
| ATOM | 1480 | CB | ARG | A | 193 | 12.410 | −76.526 | −22.970 | 1.00 | 40.38 | C |
| ATOM | 1481 | CG | ARG | A | 193 | 12.101 | −75.308 | −22.151 | 1.00 | 52.06 | C |
| ATOM | 1482 | CD | ARG | A | 193 | 12.595 | −74.035 | −22.763 | 1.00 | 53.51 | C |
| ATOM | 1483 | NE | ARG | A | 193 | 11.848 | −73.552 | −23.919 | 1.00 | 42.43 | N |
| ATOM | 1484 | CZ | ARG | A | 193 | 12.394 | −72.623 | −24.683 | 1.00 | 39.66 | C |
| ATOM | 1485 | NH1 | ARG | A | 193 | 13.598 | −72.193 | −24.362 | 1.00 | 40.87 | N |
| ATOM | 1486 | NH2 | ARG | A | 193 | 11.801 | −72.141 | −25.740 | 1.00 | 37.40 | N |
| ATOM | 1487 | C | ARG | A | 193 | 10.086 | −77.060 | −22.150 | 1.00 | 33.52 | C |
| ATOM | 1488 | O | ARG | A | 193 | 9.901 | −76.711 | −20.996 | 1.00 | 31.02 | O |
| ATOM | 1489 | N | THR | A | 194 | 9.146 | −76.945 | −23.073 | 1.00 | 34.53 | N |
| ATOM | 1490 | CA | THR | A | 194 | 7.814 | −76.480 | −22.750 | 1.00 | 32.01 | C |
| ATOM | 1491 | CB | THR | A | 194 | 7.076 | −76.064 | −24.015 | 1.00 | 30.50 | C |
| ATOM | 1492 | OG1 | THR | A | 194 | 7.798 | −75.009 | −24.654 | 1.00 | 27.41 | O |
| ATOM | 1493 | CG2 | THR | A | 194 | 5.678 | −75.608 | −23.687 | 1.00 | 28.74 | C |
| ATOM | 1494 | C | THR | A | 194 | 7.057 | −77.580 | −22.017 | 1.00 | 32.98 | C |
| ATOM | 1495 | O | THR | A | 194 | 6.419 | −77.338 | −20.997 | 1.00 | 36.47 | O |
| ATOM | 1496 | N | LEU | A | 195 | 7.162 | −78.793 | −22.531 | 1.00 | 32.61 | N |
| ATOM | 1497 | CA | LEU | A | 195 | 6.520 | −79.934 | −21.918 | 1.00 | 31.20 | C |
| ATOM | 1498 | CB | LEU | A | 195 | 6.820 | −81.205 | −22.719 | 1.00 | 28.87 | C |
| ATOM | 1499 | CG | LEU | A | 195 | 6.080 | −81.448 | −24.049 | 1.00 | 29.37 | C |
| ATOM | 1500 | CD1 | LEU | A | 195 | 6.746 | −82.524 | −24.885 | 1.00 | 24.51 | C |
| ATOM | 1501 | CD2 | LEU | A | 195 | 4.626 | −81.837 | −23.823 | 1.00 | 25.05 | C |
| ATOM | 1502 | C | LEU | A | 195 | 7.004 | −80.102 | −20.481 | 1.00 | 34.23 | C |
| ATOM | 1503 | O | LEU | A | 195 | 6.219 | −80.505 | −19.604 | 1.00 | 30.54 | O |
| ATOM | 1504 | N | CYS | A | 196 | 8.300 | −79.824 | −20.267 | 1.00 | 35.78 | N |
| ATOM | 1505 | CA | CYS | A | 196 | 8.961 | −79.986 | −18.960 | 1.00 | 38.55 | C |
| ATOM | 1506 | CB | CYS | A | 196 | 10.461 | −80.258 | −19.134 | 1.00 | 38.55 | C |
| ATOM | 1507 | SG | CYS | A | 196 | 10.959 | −81.866 | −19.794 | 1.00 | 35.83 | S |
| ATOM | 1508 | C | CYS | A | 196 | 8.807 | −78.721 | −18.102 | 1.00 | 42.41 | C |
| ATOM | 1509 | O | CYS | A | 196 | 9.459 | −78.577 | −17.066 | 1.00 | 42.97 | O |
| ATOM | 1510 | N | ASP | A | 197 | 7.961 | −77.804 | −18.565 | 1.00 | 42.95 | N |
| ATOM | 1511 | CA | ASP | A | 197 | 7.610 | −76.597 | −17.841 | 1.00 | 39.07 | C |
| ATOM | 1512 | CB | ASP | A | 197 | 6.753 | −76.939 | −16.619 | 1.00 | 40.51 | C |
| ATOM | 1513 | CG | ASP | A | 197 | 5.838 | −75.796 | −16.202 | 1.00 | 45.90 | C |
| ATOM | 1514 | OD1 | ASP | A | 197 | 5.836 | −74.715 | −16.846 | 1.00 | 44.05 | O |
| ATOM | 1515 | OD2 | ASP | A | 197 | 5.102 | −75.999 | −15.216 | 1.00 | 51.60 | O |
| ATOM | 1516 | C | ASP | A | 197 | 8.826 | −75.737 | −17.462 | 1.00 | 35.39 | C |
| ATOM | 1517 | O | ASP | A | 197 | 8.960 | −75.281 | −16.325 | 1.00 | 30.56 | O |
| ATOM | 1518 | N | TYR | A | 198 | 9.679 | −75.491 | −18.444 | 1.00 | 34.02 | N |
| ATOM | 1519 | CA | TYR | A | 198 | 10.859 | −74.524 | −18.273 | 1.00 | 35.09 | C |
| ATOM | 1520 | CB | TYR | A | 198 | 10.422 | −73.153 | −18.202 | 1.00 | 33.06 | C |
| ATOM | 1521 | CG | TYR | A | 198 | 10.056 | −72.566 | −19.524 | 1.00 | 33.43 | C |
| ATOM | 1522 | CD1 | TYR | A | 198 | 8.779 | −72.751 | −20.040 | 1.00 | 33.31 | C |
| ATOM | 1523 | CE1 | TYR | A | 198 | 8.423 | −72.236 | −21.264 | 1.00 | 33.44 | C |
| ATOM | 1524 | CZ | TYR | A | 198 | 9.325 | −71.512 | −21.991 | 1.00 | 33.07 | C |
| ATOM | 1525 | OH | TYR | A | 198 | 8.902 | −71.314 | −23.208 | 1.00 | 35.42 | O |
| ATOM | 1526 | CE2 | TYR | A | 198 | 10.619 | −71.314 | −21.516 | 1.00 | 33.96 | C |
| ATOM | 1527 | CD2 | TYR | A | 198 | 10.980 | −71.337 | −20.278 | 1.00 | 32.27 | C |
| ATOM | 1528 | C | TYR | A | 198 | 11.702 | −74.915 | −17.033 | 1.00 | 32.91 | C |
| ATOM | 1529 | O | TYR | A | 198 | 12.178 | −73.983 | −16.424 | 1.00 | 35.52 | O |
| ATOM | 1530 | N | SER | A | 199 | 11.840 | −76.170 | −16.614 | 1.00 | 32.75 | N |
| ATOM | 1531 | CA | SER | A | 199 | 12.571 | −76.474 | −15.388 | 1.00 | 33.42 | C |
| ATOM | 1532 | CB | SER | A | 199 | 12.671 | −77.985 | −15.198 | 1.00 | 33.62 | C |
| ATOM | 1533 | OG | SER | A | 199 | 13.722 | −78.235 | −14.281 | 1.00 | 35.25 | O |
| ATOM | 1534 | C | SER | A | 199 | 13.997 | −75.850 | −15.438 | 1.00 | 35.64 | C |
| ATOM | 1535 | O | SER | A | 199 | 14.651 | −75.877 | −16.480 | 1.00 | 33.93 | O |
| ATOM | 1536 | N | LYS | A | 200 | 14.461 | −75.270 | −14.333 | 1.00 | 35.39 | N |
| ATOM | 1537 | CA | LYS | A | 200 | 15.756 | −74.593 | −14.336 | 1.00 | 36.26 | C |
| ATOM | 1538 | CB | LYS | A | 200 | 15.923 | −73.725 | −13.080 | 1.00 | 39.09 | C |
| ATOM | 1539 | CG | LYS | A | 200 | 16.065 | −74.529 | −11.789 | 1.00 | 46.57 | C |
| ATOM | 1540 | CD | LYS | A | 200 | 16.182 | −73.630 | −10.565 | 1.00 | 54.64 | C |
| ATOM | 1541 | CE | LYS | A | 200 | 16.483 | −74.447 | −9.310 | 1.00 | 40.60 | O |
| ATOM | 1542 | NZ | LYS | A | 200 | 16.119 | −73.716 | −8.055 | 1.00 | 39.80 | N |
| ATOM | 1543 | C | LYS | A | 200 | 16.868 | −75.635 | −14.447 | 1.00 | 34.37 | C |
| ATOM | 1544 | O | LYS | A | 200 | 18.039 | −75.292 | −14.625 | 1.00 | 37.42 | O |
| ATOM | 1545 | N | ARG | A | 201 | 16.484 | −76.907 | −14.360 | 1.00 | 30.65 | N |
| ATOM | 1546 | CA | ARG | A | 201 | 17.440 | −78.018 | −14.330 | 1.00 | 30.00 | C |
| ATOM | 1547 | CB | ARG | A | 201 | 16.947 | −79.129 | −13.410 | 1.00 | 36.85 | C |
| ATOM | 1548 | CG | ARG | A | 201 | 17.032 | −78.765 | −11.950 | 1.00 | 30.38 | C |
| ATOM | 1549 | CD | ARG | A | 201 | 16.538 | −79.904 | −11.087 | 1.00 | 34.56 | C |
| ATOM | 1550 | NE | ARG | A | 201 | 17.518 | −80.988 | −10.976 | 1.00 | 19.82 | N |
| ATOM | 1551 | CZ | ARG | A | 201 | 17.238 | −82.192 | −10.476 | 1.00 | 42.57 | C |
| ATOM | 1552 | NH1 | ARG | A | 201 | 16.002 | −82.467 | −10.050 | 1.00 | 40.85 | N |
| ATOM | 1553 | NH2 | ARG | A | 201 | 18.186 | −83.125 | −10.412 | 1.00 | 42.39 | N |
| ATOM | 1554 | C | ARG | A | 201 | 17.745 | −78.595 | −15.721 | 1.00 | 29.66 | C |
| ATOM | 1555 | O | ARG | A | 201 | 18.548 | −79.509 | −15.867 | 1.00 | 32.55 | O |
| ATOM | 1556 | N | ILE | A | 202 | 17.119 | −78.043 | −16.741 | 1.00 | 27.78 | N |
| ATOM | 1557 | CA | ILE | A | 202 | 17.250 | −78.579 | −18.067 | 1.00 | 26.81 | C |
| ATOM | 1558 | CB | ILE | A | 202 | 15.884 | −79.009 | −18.605 | 1.00 | 28.34 | C |
| ATOM | 1559 | CG1 | ILE | A | 202 | 15.303 | −80.094 | −17.676 | 1.00 | 26.98 | C |
| ATOM | 1560 | CD1 | ILE | A | 202 | 13.813 | −80.400 | −17.854 | 1.00 | 28.62 | C |
| ATOM | 1561 | CG2 | ILE | A | 202 | 16.017 | −79.458 | −20.053 | 1.00 | 27.83 | C |
| ATOM | 1562 | C | ILE | A | 202 | 17.862 | −77.513 | −18.928 | 1.00 | 27.66 | C |
| ATOM | 1563 | O | ILE | A | 202 | 17.326 | −76.422 | −19.049 | 1.00 | 28.92 | O |
| ATOM | 1564 | N | ALA | A | 203 | 19.034 | −77.809 | −19.484 | 1.00 | 28.89 | N |
| ATOM | 1565 | CA | ALA | A | 203 | 19.783 | −76.820 | −20.225 | 1.00 | 26.69 | C |
| ATOM | 1566 | CB | ALA | A | 203 | 20.939 | −76.355 | −19.393 | 1.00 | 26.16 | C |
| ATOM | 1567 | C | ALA | A | 203 | 20.220 | −77.410 | −21.564 | 1.00 | 27.71 | C |
| ATOM | 1568 | O | ALA | A | 203 | 19.976 | −78.585 | −21.812 | 1.00 | 27.85 | O |
| ATOM | 1569 | N | VAL | A | 204 | 20.801 | −76.597 | −22.449 | 1.00 | 30.11 | N |
| ATOM | 1570 | CA | VAL | A | 204 | 21.213 | −77.068 | −23.785 | 1.00 | 28.43 | C |
| ATOM | 1571 | CB | VAL | A | 204 | 20.834 | −76.053 | −24.877 | 1.00 | 28.01 | C |
| ATOM | 1572 | CG1 | VAL | A | 204 | 21.169 | −76.573 | −26.286 | 1.00 | 25.08 | C |
| ATOM | 1573 | CG2 | VAL | A | 204 | 19.325 | −75.741 | −24.773 | 1.00 | 32.32 | C |
| ATOM | 1574 | C | VAL | A | 204 | 22.721 | −77.406 | −23.835 | 1.00 | 33.56 | C |
| ATOM | 1575 | O | VAL | A | 204 | 23.588 | −76.639 | −23.359 | 1.00 | 30.75 | O |
| ATOM | 1576 | N | ALA | A | 205 | 23.003 | −78.599 | −24.357 | 1.00 | 35.20 | N |
| ATOM | 1577 | CA | ALA | A | 205 | 24.348 | −78.995 | −24.787 | 1.00 | 33.84 | C |
| ATOM | 1578 | CB | ALA | A | 205 | 24.648 | −80.414 | −24.315 | 1.00 | 31.37 | C |
| ATOM | 1579 | C | ALA | A | 205 | 24.356 | −78.906 | −26.318 | 1.00 | 32.42 | C |
| ATOM | 1580 | O | ALA | A | 205 | 24.040 | −79.862 | −27.005 | 1.00 | 31.38 | O |
| ATOM | 1581 | N | LEU | A | 206 | 24.693 | −77.744 | −26.851 | 1.00 | 34.03 | N |
| ATOM | 1582 | CA | LEU | A | 206 | 24.529 | −77.492 | −28.274 | 1.00 | 36.06 | C |
| ATOM | 1583 | CB | LEU | A | 206 | 24.491 | −75.979 | −28.510 | 1.00 | 33.53 | C |
| ATOM | 1584 | CG | LEU | A | 206 | 24.278 | −75.390 | −29.908 | 1.00 | 35.38 | C |
| ATOM | 1585 | CD1 | LEU | A | 206 | 22.862 | −75.595 | −30.451 | 1.00 | 36.00 | C |
| ATOM | 1586 | CD2 | LEU | A | 206 | 24.617 | −73.906 | −29.878 | 1.00 | 33.64 | C |
| ATOM | 1587 | C | LEU | A | 206 | 25.637 | −78.173 | −29.089 | 1.00 | 39.89 | C |
| ATOM | 1588 | O | LEU | A | 206 | 26.818 | −77.860 | −28.908 | 1.00 | 43.36 | O |
| ATOM | 1589 | N | GLU | A | 207 | 25.260 | −79.124 | −29.951 | 1.00 | 42.83 | N |
| ATOM | 1590 | CA | GLU | A | 207 | 26.210 | −79.808 | −30.842 | 1.00 | 47.01 | C |
| ATOM | 1591 | CB | GLU | A | 207 | 25.757 | −81.228 | −31.164 | 1.00 | 49.08 | C |
| ATOM | 1592 | CG | GLU | A | 207 | 26.579 | −82.275 | −30.433 | 1.00 | 50.80 | C |
| ATOM | 1593 | CD | GLU | A | 207 | 26.464 | −83.678 | −31.000 | 1.00 | 53.64 | C |
| ATOM | 1594 | OE1 | GLU | A | 207 | 25.574 | −84.440 | −30.567 | 1.00 | 59.53 | O |
| ATOM | 1595 | OE2 | GLU | A | 207 | 27.290 | −84.042 | −31.855 | 1.00 | 52.22 | O |
| ATOM | 1596 | C | GLU | A | 207 | 26.491 | −79.050 | −32.138 | 1.00 | 49.28 | C |
| ATOM | 1597 | O | GLU | A | 207 | 25.581 | −78.812 | −37.935 | 1.00 | 50.07 | O |
| ATOM | 1598 | N | ILE | A | 208 | 27.758 | −78.685 | −32.335 | 1.00 | 46.63 | N |
| ATOM | 1599 | CA | ILE | A | 208 | 28.197 | −77.944 | −33.525 | 1.00 | 50.01 | C |
| ATOM | 1600 | CB | ILE | A | 208 | 29.200 | −76.837 | −33.133 | 1.00 | 51.62 | C |
| ATOM | 1601 | CG1 | ILE | A | 208 | 28.591 | −75.968 | −32.034 | 1.00 | 50.90 | C |
| ATOM | 1602 | CD1 | ILE | A | 208 | 29.607 | −75.096 | −31.339 | 1.00 | 56.71 | C |
| ATOM | 1603 | CG2 | ILE | A | 208 | 29.585 | −75.968 | −34.328 | 1.00 | 45.22 | C |
| ATOM | 1604 | C | ILE | A | 208 | 28.769 | −78.861 | −34.620 | 1.00 | 49.82 | C |
| ATOM | 1605 | O | ILE | A | 208 | 29.522 | −79.787 | −34.341 | 1.00 | 49.77 | O |
| ATOM | 1606 | N | GLY | A | 209 | 28.380 | −78.612 | −35.865 | 1.00 | 50.94 | N |
| ATOM | 1607 | CA | GLY | A | 209 | 28.795 | −79.452 | −36.991 | 1.00 | 53.08 | C |
| ATOM | 1608 | C | GLY | A | 209 | 29.771 | −78.796 | −37.964 | 1.00 | 56.16 | C |
| ATOM | 1609 | O | GLY | A | 209 | 30.042 | −77.584 | −37.895 | 1.00 | 51.38 | O |
| ATOM | 1610 | N | ALA | A | 210 | 30.300 | −79.618 | −38.875 | 1.00 | 61.54 | N |
| ATOM | 1611 | CA | ALA | A | 210 | 31.269 | −79.178 | −39.879 | 1.00 | 60.31 | C |
| ATOM | 1612 | CB | ALA | A | 210 | 31.625 | −80.323 | −40.824 | 1.00 | 60.56 | C |
| ATOM | 1613 | C | ALA | A | 210 | 30.702 | −77.992 | −40.632 | 1.00 | 62.80 | C |
| ATOM | 1614 | O | ALA | A | 210 | 31.355 | −76.957 | −40.752 | 1.00 | 65.39 | O |
| ATOM | 1615 | N | ASP | A | 211 | 29.458 | −78.142 | −41.080 | 1.00 | 63.65 | N |
| ATOM | 1616 | CA | ASP | A | 211 | 28.722 | −77.100 | −41.771 | 1.00 | 67.04 | C |
| ATOM | 1617 | CB | ASP | A | 211 | 27.819 | −77.733 | −42.835 | 1.00 | 74.66 | C |
| ATOM | 1618 | CG | ASP | A | 211 | 28.507 | −77.886 | −44.179 | 1.00 | 80.02 | C |
| ATOM | 1619 | OD1 | ASP | A | 211 | 29.711 | −78.240 | −44.232 | 1.00 | 84.34 | O |
| ATOM | 1620 | OD2 | ASP | A | 211 | 27.826 | −77.652 | −45.196 | 1.00 | 81.89 | O |
| ATOM | 1621 | C | ASP | A | 211 | 27.857 | −76.330 | −40.794 | 1.00 | 65.06 | C |
| ATOM | 1622 | O | ASP | A | 211 | 26.860 | −76.358 | −40.303 | 1.00 | 64.61 | O |
| ATOM | 1623 | N | LEU | A | 212 | 28.230 | −75.390 | −40.505 | 1.00 | 62.01 | N |
| ATOM | 1624 | CA | LEU | A | 212 | 27.365 | −74.244 | −39.708 | 1.00 | 65.23 | C |
| ATOM | 1625 | CB | LEU | A | 212 | 28.000 | −72.392 | −39.362 | 1.00 | 60.58 | C |
| ATOM | 1626 | CG | LEU | A | 212 | 29.244 | −72.312 | −38.475 | 1.00 | 59.84 | C |
| ATOM | 1627 | CD1 | LEU | A | 212 | 29.402 | −71.398 | −37.936 | 1.00 | 59.85 | C |
| ATOM | 1628 | CD2 | LEU | A | 212 | 29.194 | −73.111 | −37.330 | 1.00 | 59.84 | C |
| ATOM | 1629 | C | LEU | A | 212 | 26.138 | −74.009 | −40.543 | 1.00 | 75.57 | C |
| ATOM | 1630 | O | LEU | A | 212 | 26.250 | −73.320 | −41.758 | 1.00 | 83.07 | O |
| ATOM | 1631 | N | PRO | A | 213 | 24.958 | −74.021 | −39.906 | 1.00 | 81.11 | N |
| ATOM | 1632 | CA | PRO | A | 213 | 23.761 | −73.735 | −40.669 | 1.00 | 81.74 | C |
| ATOM | 1633 | CB | PRO | A | 213 | 22.649 | −74.965 | −39.680 | 1.00 | 81.91 | C |
| ATOM | 1634 | CG | PRO | A | 213 | 23.239 | −73.687 | −38.361 | 1.00 | 82.11 | C |
| ATOM | 1635 | CD | PRO | A | 213 | 24.687 | −74.074 | −38.455 | 1.00 | 78.56 | C |
| ATOM | 1636 | C | PRO | A | 213 | 23.774 | −72.246 | −40.967 | 1.00 | 84.53 | C |
| ATOM | 1637 | O | PRO | A | 213 | 24.734 | −71.356 | −40.594 | 1.00 | 80.25 | O |
| ATOM | 1638 | N | SER | A | 214 | 22.725 | −71.759 | −41.626 | 1.00 | 84.69 | N |
| ATOM | 1639 | CA | SER | A | 214 | 22.531 | −70.326 | −41.810 | 1.00 | 82.62 | C |
| ATOM | 1640 | CB | SER | A | 214 | 21.175 | −70.057 | −42.457 | 1.00 | 87.35 | C |
| ATOM | 1641 | OG | SER | A | 214 | 21.224 | −70.314 | −43.849 | 1.00 | 88.05 | O |
| ATOM | 1642 | C | SER | A | 214 | 22.664 | −69.572 | −40.482 | 1.00 | 73.33 | C |
| ATOM | 1643 | O | SER | A | 214 | 22.487 | −70.158 | −39.409 | 1.00 | 72.81 | O |
| ATOM | 1644 | N | AASN | A | 215 | 22.991 | −63.286 | −40.565 | 0.50 | 72.89 | N |
| ATOM | 1645 | N | BASN | A | 215 | 22.987 | −68.282 | −40.571 | 0.50 | 73.84 | N |
| ATOM | 1646 | CA | AASN | A | 215 | 23.177 | −67.458 | −39.377 | 0.50 | 69.84 | C |
| ATOM | 1647 | CA | BASN | A | 215 | 23.176 | −67.427 | −39.397 | 0.50 | 70.80 | C |
| ATOM | 1648 | CB | AASN | A | 215 | 23.741 | −66.082 | −39.735 | 0.50 | 64.47 | C |
| ATOM | 1649 | CB | BASN | A | 215 | 23.656 | −66.026 | −39.794 | 0.50 | 66.68 | C |
| ATOM | 1650 | CG | AASN | A | 215 | 25.234 | −65.991 | −39.493 | 0.50 | 62.58 | C |
| ATOM | 1651 | CG | BASN | A | 215 | 23.653 | −65.054 | −38.623 | 0.50 | 65.99 | C |
| ATOM | 1652 | OD1 | AASN | A | 215 | 25.945 | −66.994 | −39.561 | 0.50 | 59.06 | O |
| ATOM | 1653 | OD1 | BASN | A | 215 | 24.575 | −64.255 | −38.453 | 0.50 | 63.08 | O |
| ATOM | 1654 | ND2 | AASN | A | 215 | 25.716 | −64.190 | −39.194 | 0.50 | 60.09 | N |
| ATOM | 1655 | ND2 | BASN | A | 215 | 22.615 | −65.125 | −37.806 | 0.50 | 64.16 | N |
| ATOM | 1656 | C | AASN | A | 215 | 21.917 | −67.312 | −38.550 | 0.50 | 70.25 | C |
| ATOM | 1657 | C | BASN | A | 215 | 21.920 | −67.321 | −38.555 | 0.50 | 70.84 | C |
| ATOM | 1658 | O | AASN | A | 215 | 21.995 | −67.146 | −37.336 | 0.50 | 70.82 | O |
| ATOM | 1659 | O | BASN | A | 215 | 22.006 | −67.188 | −37.337 | 0.50 | 71.01 | O |
| ATOM | 1660 | N | HIS | A | 216 | 20.761 | −67.392 | −39.210 | 1.00 | 72.25 | N |
| ATOM | 1661 | CA | HIS | A | 216 | 19.472 | −67.265 | −38.518 | 1.00 | 74.24 | C |
| ATOM | 1662 | CB | HIS | A | 216 | 18.289 | −66.996 | −39.484 | 1.00 | 70.91 | C |
| ATOM | 1663 | CG | HIS | A | 216 | 17.870 | −68.174 | −40.318 | 1.00 | 79.61 | C |
| ATOM | 1664 | ND1 | HIS | A | 216 | 16.718 | −68.896 | −40.068 | 1.00 | 77.20 | N |
| ATOM | 1665 | CE1 | HIS | A | 216 | 16.590 | −69.847 | −40.977 | 1.00 | 73.60 | C |
| ATOM | 1666 | NE2 | HIS | A | 216 | 17.612 | −69.766 | −41.812 | 1.00 | 74.72 | N |
| ATOM | 1667 | CD2 | HIS | A | 216 | 18.421 | −68.723 | −41.429 | 1.00 | 76.91 | C |
| ATOM | 1668 | C | HIS | A | 216 | 19.230 | −68.416 | −37.537 | 1.00 | 70.18 | C |
| ATOM | 1669 | O | HIS | A | 216 | 18.808 | −68.203 | −36.405 | 1.00 | 68.50 | O |
| ATOM | 1670 | N | VAL | A | 217 | 19.570 | −69.623 | −37.963 | 1.00 | 70.58 | N |
| ATOM | 1671 | CA | VAL | A | 217 | 19.495 | −70.797 | −37.111 | 1.00 | 72.75 | C |
| ATOM | 1672 | CB | VAL | A | 217 | 19.873 | −72.079 | −37.900 | 1.00 | 32.25 | C |
| ATOM | 1673 | CG1 | VAL | A | 217 | 19.478 | −73.339 | −37.130 | 1.00 | 85.79 | C |
| ATOM | 1674 | CG2 | VAL | A | 217 | 19.232 | −72.077 | −39.292 | 1.00 | 77.22 | C |
| ATOM | 1675 | C | VAL | A | 217 | 20.370 | −70.620 | −35.855 | 1.00 | 76.30 | C |
| ATOM | 1676 | O | VAL | A | 217 | 20.035 | −71.146 | −34.798 | 1.00 | 88.07 | O |
| ATOM | 1677 | N | ILE | A | 218 | 21.469 | −69.865 | −35.969 | 1.00 | 71.39 | N |
| ATOM | 1678 | CA | ILE | A | 218 | 22.334 | −69.512 | −34.829 | 1.00 | 63.62 | C |
| ATOM | 1679 | CB | ILE | A | 218 | 23.802 | −69.232 | −35.296 | 1.00 | 64.81 | C |
| ATOM | 1680 | CG1 | ILE | A | 218 | 24.488 | −70.535 | −35.722 | 1.00 | 66.65 | C |
| ATOM | 1681 | CD1 | ILE | A | 218 | 25.585 | −70.375 | −36.765 | 1.00 | 65.69 | C |
| ATOM | 1682 | CG2 | ILE | A | 218 | 24.642 | −68.540 | −34.221 | 1.00 | 57.45 | C |
| ATOM | 1683 | C | ILE | A | 218 | 21.753 | −69.306 | −34.094 | 1.00 | 63.80 | C |
| ATOM | 1684 | O | ILE | A | 218 | 22.251 | −67.892 | −33.045 | 1.00 | 66.39 | O |
| ATOM | 1685 | N | ASP | A | 219 | 20.711 | −67.722 | −34.667 | 1.00 | 65.81 | N |
| ATOM | 1686 | CA | ASP | A | 219 | 20.006 | −66.637 | −34.005 | 1.00 | 70.35 | C |
| ATOM | 1687 | CB | ASP | A | 219 | 19.203 | −65.788 | −35.010 | 1.00 | 68.11 | C |
| ATOM | 1688 | CG | ASP | A | 219 | 20.090 | −64.970 | −35.974 | 1.00 | 66.72 | C |
| ATOM | 1689 | OD1 | ASP | A | 219 | 21.340 | −64.890 | −35.776 | 1.00 | 56.49 | O |
| ATOM | 1690 | OD2 | ASP | A | 219 | 19.499 | −64.397 | −36.935 | 1.00 | 58.92 | O |
| ATOM | 1691 | C | ASP | A | 219 | 19.076 | −67.250 | −32.947 | 1.00 | 68.16 | C |
| ATOM | 1692 | O | ASP | A | 219 | 18.964 | −66.749 | −31.814 | 1.00 | 60.18 | O |
| ATOM | 1693 | N | ARG | A | 220 | 18.433 | −68.356 | −33.323 | 1.00 | 65.96 | N |
| ATOM | 1694 | CA | ARG | A | 220 | 17.443 | −69.022 | −32.474 | 1.00 | 64.92 | C |
| ATOM | 1695 | CB | ARG | A | 220 | 17.120 | −70.408 | −33.032 | 1.00 | 60.94 | C |
| ATOM | 1696 | CG | ARG | A | 220 | 16.152 | −70.424 | −34.199 | 1.00 | 54.97 | C |
| ATOM | 1697 | CD | ARG | A | 220 | 15.163 | −69.309 | −34.047 | 1.00 | 57.59 | C |
| ATOM | 1698 | NE | ARG | A | 220 | 13.806 | −69.737 | −34.310 | 1.00 | 63.59 | N |
| ATOM | 1699 | CZ | ARG | A | 220 | 12.841 | −69.741 | −33.393 | 1.00 | 75.12 | C |
| ATOM | 1700 | NH1 | ARG | A | 220 | 13.089 | −69.331 | −32.146 | 1.00 | 71.79 | N |
| ATOM | 1701 | NH2 | ARG | A | 220 | 11.619 | −70.144 | 33.730 | 1.00 | 81.53 | N |
| ATOM | 1702 | C | ARG | A | 220 | 17.964 | −69.190 | −31.062 | 1.00 | 67.63 | C |
| ATOM | 1703 | O | ARG | A | 220 | 17.280 | −68.872 | −30.093 | 1.00 | 74.03 | O |
| ATOM | 1704 | N | TRP | A | 221 | 19.206 | −69.661 | −30.977 | 1.00 | 56.04 | N |
| ATOM | 1705 | CA | TRP | A | 221 | 19.794 | −70.167 | −29.760 | 1.00 | 43.04 | C |
| ATOM | 1706 | CB | TRP | A | 221 | 20.920 | −71.106 | −30.145 | 1.00 | 37.72 | C |
| ATOM | 1707 | CG | TRP | A | 221 | 20.425 | −72.300 | −30.859 | 1.00 | 31.33 | C |
| ATOM | 1708 | CD1 | TRP | A | 221 | 20.512 | −72.572 | −32.200 | 1.00 | 28.18 | C |
| ATOM | 1709 | NE1 | TRP | A | 221 | 19.927 | −73.780 | −32.469 | 1.00 | 27.07 | N |
| ATOM | 1710 | CE2 | TRP | A | 221 | 19.441 | −74.306 | −31.298 | 1.00 | 28.54 | C |
| ATOM | 1711 | CD2 | TRP | A | 221 | 19.734 | −73.395 | −30.268 | 1.00 | 29.38 | C |
| ATOM | 1712 | CE3 | TRP | A | 221 | 19.325 | −73.689 | −28.966 | 1.00 | 27.88 | C |
| ATOM | 1713 | CZ3 | TRP | A | 221 | 18.650 | −74.889 | −23.732 | 1.00 | 23.60 | C |
| ATOM | 1714 | CH2 | TRP | A | 221 | 18.369 | −75.769 | −29.773 | 1.00 | 23.57 | C |
| ATOM | 1715 | CZ2 | TRP | A | 221 | 18.752 | −75.496 | −31.062 | 1.00 | 31.07 | C |
| ATOM | 1716 | C | TRP | A | 221 | 20.321 | −69.069 | −28.881 | 1.00 | 44.95 | C |
| ATOM | 1717 | O | TRP | A | 221 | 20.901 | −69.335 | −27.809 | 1.00 | 43.09 | O |
| ATOM | 1718 | N | LEU | A | 222 | 20.104 | −67.834 | −29.323 | 1.00 | 47.35 | N |
| ATOM | 1719 | CA | LEU | A | 222 | 20.752 | −66.684 | −28.685 | 1.00 | 51.38 | C |
| ATOM | 1720 | CB | LEU | A | 222 | 20.453 | −65.354 | −29.423 | 1.00 | 43.90 | C |
| ATOM | 1721 | CG | LEU | A | 222 | 20.859 | −65.288 | −30.905 | 1.00 | 53.10 | C |
| ATOM | 1722 | CD1 | LEU | A | 222 | 20.852 | −63.895 | −31.533 | 1.00 | 50.11 | C |
| ATOM | 1723 | CD2 | LEU | A | 222 | 22.219 | −65.343 | −31.145 | 1.00 | 49.10 | C |
| ATOM | 1724 | C | LEU | A | 222 | 20.492 | −66.520 | −27.160 | 1.00 | 49.41 | C |
| ATOM | 1725 | O | LEU | A | 222 | 21.458 | −66.537 | −26.345 | 1.00 | 52.76 | O |
| ATOM | 1726 | N | GLY | A | 223 | 19.213 | −66.620 | −26.775 | 1.00 | 37.97 | N |
| ATOM | 1727 | CA | GLY | A | 223 | 18.853 | −66.437 | −25.374 | 1.00 | 32.01 | C |
| ATOM | 1728 | C | GLY | A | 223 | 18.576 | −67.705 | −24.633 | 1.00 | 30.95 | C |
| ATOM | 1729 | O | GLY | A | 223 | 18.157 | −67.563 | −23.479 | 1.00 | 37.25 | O |
| ATOM | 1730 | N | GLU | A | 224 | 18.797 | −68.345 | −25.279 | 1.00 | 31.34 | N |
| ATOM | 1731 | CA | GLU | A | 224 | 18.516 | −70.148 | −24.649 | 1.00 | 31.27 | C |
| ATOM | 1732 | CB | GLU | A | 224 | 18.448 | −71.222 | −25.721 | 1.00 | 22.02 | C |
| ATOM | 1733 | CG | GLU | A | 224 | 17.404 | −70.345 | −26.779 | 1.00 | 31.22 | C |
| ATOM | 1734 | CD | GLU | A | 224 | 15.971 | −70.377 | −26.227 | 1.00 | 32.48 | C |
| ATOM | 1735 | OE1 | GLU | A | 224 | 15.073 | −70.320 | −26.938 | 1.00 | 30.80 | O |
| ATOM | 1736 | OE2 | GLU | A | 224 | 15.737 | −71.439 | −25.094 | 1.00 | 30.01 | O |
| ATOM | 1737 | C | GLU | A | 224 | 19.590 | −70.330 | −23.588 | 1.00 | 32.08 | C |
| ATOM | 1738 | O | GLU | A | 224 | 20.720 | −70.329 | −23.624 | 1.00 | 30.91 | O |
| ATOM | 1739 | N | PRO | A | 225 | 19.210 | −71.392 | −22.627 | 1.00 | 31.78 | N |
| ATOM | 1740 | CA | PRO | A | 225 | 20.108 | −71.323 | −21.529 | 1.00 | 31.35 | C |
| ATOM | 1741 | CB | PRO | A | 225 | 19.171 | −72.603 | −20.583 | 1.00 | 28.54 | C |
| ATOM | 1742 | CG | PRO | A | 225 | 18.033 | −73.023 | −21.457 | 1.00 | 28.62 | C |
| ATOM | 1743 | CD | PRO | A | 225 | 17.829 | −71.398 | −22.433 | 1.00 | 30.00 | C |
| ATOM | 1744 | C | PRO | A | 225 | 21.238 | −72.148 | −21.970 | 1.00 | 30.99 | C |
| ATOM | 1745 | O | PRO | A | 225 | 21.276 | −73.910 | −21.579 | 1.00 | 31.10 | O |
| ATOM | 1746 | N | ILE | A | 226 | 22.159 | −72.240 | −22.769 | 1.00 | 34.57 | N |
| ATOM | 1747 | CA | ILE | A | 226 | 23.262 | −73.081 | −23.212 | 1.00 | 35.42 | C |
| ATOM | 1748 | CB | ILE | A | 226 | 23.839 | −72.626 | −24.551 | 1.00 | 35.10 | C |
| ATOM | 1749 | CG1 | ILE | A | 226 | 22.704 | −72.806 | −25.562 | 1.00 | 32.92 | C |
| ATOM | 1750 | CD1 | ILE | A | 226 | 23.023 | −71.716 | −26.812 | 1.00 | 34.64 | C |
| ATOM | 1751 | CG2 | ILE | A | 226 | 24.819 | −73.683 | −25.062 | 1.00 | 38.03 | C |
| ATOM | 1752 | C | ILE | A | 226 | 24.307 | −73.220 | −22.110 | 1.00 | 36.00 | C |
| ATOM | 1753 | O | ILE | A | 226 | 24.899 | −72.245 | −21.673 | 1.00 | 41.18 | O |
| ATOM | 1754 | N | LYS | A | 227 | 24.474 | −74.440 | −21.626 | 1.00 | 37.18 | N |
| ATOM | 1755 | CA | LYS | A | 227 | 25.465 | −74.728 | −20.601 | 1.00 | 41.41 | C |
| ATOM | 1756 | CB | LYS | A | 227 | 24.881 | −75.627 | −19.507 | 1.00 | 43.21 | C |
| ATOM | 1757 | CG | LYS | A | 227 | 24.143 | −74.877 | −18.412 | 1.00 | 47.10 | C |
| ATOM | 1758 | CD | LYS | A | 227 | 24.891 | −73.619 | −17.999 | 1.00 | 51.44 | C |
| ATOM | 1759 | CE | LYS | A | 227 | 24.308 | −73.001 | −16.735 | 1.00 | 58.01 | C |
| ATOM | 1760 | NZ | LYS | A | 227 | 25.168 | −71.196 | −16.214 | 1.00 | 57.97 | N |
| ATOM | 1761 | C | LYS | A | 227 | 26.723 | −75.368 | −21.167 | 1.00 | 39.70 | C |
| ATOM | 1762 | O | LYS | A | 227 | 27.773 | −75.301 | −20.543 | 1.00 | 44.10 | O |
| ATOM | 1763 | N | ALA | A | 228 | 76.618 | −76.002 | −22.334 | 1.00 | 38.39 | N |
| ATOM | 1764 | CA | ALA | A | 228 | 27.788 | −76.622 | −22.966 | 1.00 | 37.92 | C |
| ATOM | 1765 | CA | ALA | A | 228 | 27.963 | −78.068 | −22.526 | 1.00 | 33.72 | C |
| ATOM | 1766 | C | ALA | A | 228 | 27.682 | −76.551 | −24.458 | 1.00 | 38.26 | C |
| ATOM | 1767 | O | ALA | A | 228 | 26.582 | −76.532 | −24.998 | 1.00 | 37.63 | O |
| ATOM | 1768 | N | ALA | A | 229 | 28.836 | −76.510 | −25.121 | 1.00 | 36.09 | N |
| ATOM | 1769 | CA | ALA | A | 229 | 26.919 | −76.651 | −26.578 | 1.00 | 34.45 | C |
| ATOM | 1770 | CB | ALA | A | 229 | 29.406 | −75.366 | −27.205 | 1.00 | 35.38 | C |
| ATOM | 1771 | C | ALA | A | 229 | 29.831 | −77.817 | −26.960 | 1.00 | 38.32 | C |
| ATOM | 1772 | O | ALA | A | 229 | 30.971 | −77.920 | −26.488 | 1.00 | 40.01 | O |
| ATOM | 1773 | N | ILE | A | 230 | 29.317 | −78.702 | −27.803 | 1.00 | 42.50 | N |
| ATOM | 1774 | CA | ILE | A | 230 | 30.009 | −79.937 | −28.158 | 1.00 | 40.75 | C |
| ATOM | 1775 | CB | ILE | A | 230 | 29.043 | −81.121 | −28.171 | 1.00 | 37.16 | C |
| ATOM | 1776 | CG1 | ILE | A | 230 | 28.691 | −81.498 | −26.740 | 1.00 | 39.26 | C |
| ATOM | 1777 | CD1 | ILE | A | 230 | 27.357 | −82.225 | −26.606 | 1.00 | 39.55 | C |
| ATOM | 1778 | CG2 | ILE | A | 230 | 29.654 | −82.235 | −28.830 | 1.00 | 36.77 | C |
| ATOM | 1779 | C | ILE | A | 230 | 30.726 | −79.777 | −29.496 | 1.00 | 44.35 | C |
| ATOM | 1780 | O | ILE | A | 230 | 30.107 | −79.442 | −30.504 | 1.00 | 45.35 | O |
| ATOM | 1781 | N | LEU | A | 231 | 32.042 | −79.595 | −29.482 | 1.00 | 45.53 | N |
| ATOM | 1782 | CA | LEU | A | 231 | 32.893 | −79.808 | −30.676 | 1.00 | 43.52 | C |
| ATOM | 1783 | CB | LEU | A | 231 | 34.045 | −78.863 | −30.362 | 1.00 | 40.56 | C |
| ATOM | 1784 | CG | LEU | A | 231 | 33.610 | −77.543 | −29.726 | 1.00 | 40.18 | C |
| ATOM | 1785 | CD1 | LEU | A | 231 | 34.806 | −76.715 | −29.310 | 1.00 | 43.09 | C |
| ATOM | 1786 | CD2 | LEU | A | 231 | 32.748 | −76.749 | −30.691 | 1.00 | 40.21 | C |
| ATOM | 1787 | C | LEU | A | 231 | 33.437 | −81.144 | −31.100 | 1.00 | 41.46 | C |
| ATOM | 1788 | G | LEU | A | 231 | 34.284 | −81.696 | −30.416 | 1.00 | 42.94 | O |
| ATOM | 1789 | N | PRO | A | 232 | 32.917 | −81.707 | −32.207 | 1.00 | 43.89 | N |
| ATOM | 1790 | CA | PRO | A | 232 | 33.474 | −82.962 | −32.707 | 1.00 | 44.44 | C |
| ATOM | 1791 | CB | PRO | A | 232 | 32.466 | −83.398 | −33.782 | 1.00 | 43.93 | C |
| ATOM | 1792 | CG | PRO | A | 232 | 31.250 | −82.586 | −33.532 | 1.00 | 48.95 | C |
| ATOM | 1793 | CD | PRO | A | 232 | 31.767 | −81.277 | −33.003 | 1.00 | 46.26 | C |
| ATOM | 1794 | C | PRO | A | 232 | 34.839 | −82.720 | −33.333 | 1.00 | 41.40 | C |
| ATOM | 1795 | O | PRO | A | 232 | 35.073 | −81.681 | −33.968 | 1.00 | 39.17 | O |
| ATOM | 1796 | N | THR | A | 233 | 35.743 | −83.668 | −33.159 | 1.00 | 42.40 | N |
| ATOM | 1797 | CA | THR | A | 233 | 37.072 | −33.532 | −33.773 | 1.00 | 42.68 | C |
| ATOM | 1798 | CB | THR | A | 233 | 37.967 | −84.715 | −33.409 | 1.00 | 39.37 | C |
| ATOM | 1799 | CG1 | THR | A | 733 | 37.268 | −85.937 | −33.673 | 1.00 | 40.59 | O |
| ATOM | 1800 | CG2 | THR | A | 233 | 38.316 | −84.649 | −31.926 | 1.00 | 36.65 | C |
| ATOM | 1801 | C | THR | A | 233 | 36.957 | −83.303 | −35.290 | 1.00 | 43.74 | C |
| ATOM | 1802 | O | THR | A | 233 | 37.670 | −82.465 | −35.837 | 1.00 | 42.48 | O |
| ATOM | 1803 | N | SER | A | 234 | 36.013 | −84.011 | −35.926 | 1.00 | 44.73 | N |
| ATOM | 1804 | CA | SER | A | 234 | 35.598 | −83.817 | −37.336 | 1.00 | 47.07 | C |
| ATOM | 1805 | CB | SER | A | 234 | 34.176 | −84.358 | −37.544 | 1.00 | 50.31 | C |
| ATOM | 1806 | CG | SER | A | 234 | 34.088 | −85.724 | −37.216 | 1.00 | 60.95 | O |
| ATOM | 1807 | C | SER | A | 234 | 35.555 | −82.400 | −37.907 | 1.00 | 50.45 | C |
| ATOM | 1808 | O | SER | A | 234 | 35.598 | −82.234 | −39.133 | 1.00 | 51.24 | O |
| ATOM | 1809 | N | ILE | A | 235 | 35.394 | −81.380 | −37.067 | 1.00 | 49.02 | N |
| ATOM | 1810 | CA | ILE | A | 235 | 35.112 | −80.050 | −37.619 | 1.00 | 51.95 | C |
| ATOM | 1811 | CB | ILE | A | 235 | 33.957 | −79.318 | −36.890 | 1.00 | 50.52 | C |
| ATOM | 1812 | CG1 | ILE | A | 235 | 34.422 | −78.790 | −35.533 | 1.00 | 55.67 | C |
| ATOM | 1813 | CD1 | ILE | A | 235 | 33.521 | −77.729 | −34.946 | 1.00 | 55.20 | C |
| ATOM | 1814 | CG2 | ILE | A | 235 | 32.761 | −80.235 | −36.734 | 1.00 | 45.70 | C |
| ATOM | 1815 | C | ILE | A | 235 | 36.358 | −79.191 | −37.689 | 1.00 | 52.59 | O |
| ATOM | 1816 | O | ILE | A | 235 | 36.373 | −78.143 | −38.340 | 1.00 | 55.90 | N |
| ATOM | 1817 | N | PHE | A | 236 | 37.410 | −79.646 | −37.024 | 1.00 | 57.17 | C |
| ATOM | 1818 | C | PHE | A | 236 | 36.676 | −76.930 | −37.047 | 1.00 | 56.14 | C |
| ATOM | 1819 | CB | PHE | A | 236 | 39.578 | −79.412 | −35.924 | 1.00 | 51.41 | C |
| ATOM | 1820 | CG | PHE | A | 236 | 39.106 | −79.005 | −34.566 | 1.00 | 49.55 | C |
| ATOM | 1821 | CD1 | PHE | A | 236 | 38.107 | −79.729 | −33.913 | 1.00 | 48.56 | C |
| ATOM | 1822 | CE1 | PHE | A | 236 | 37.667 | −79.351 | −32.651 | 1.00 | 47.35 | C |
| ATOM | 1823 | CZ | PHE | A | 236 | 38.229 | −78.249 | −32.023 | 1.00 | 47.28 | C |
| ATOM | 1824 | CE2 | PHE | A | 236 | 39.230 | −77.519 | −32.658 | 1.00 | 48.00 | C |
| ATOM | 1825 | CD2 | PHE | A | 236 | 39.663 | −77.897 | −33.925 | 1.00 | 49.63 | C |
| ATOM | 1826 | C | PHE | A | 236 | 39.362 | −79.125 | −38.381 | 1.00 | 58.52 | C |
| ATOM | 1827 | O | PHE | A | 236 | 39.364 | −80.214 | −38.948 | 1.00 | 61.95 | O |
| ATOM | 1828 | N | LEU | A | 237 | 39.912 | −78.035 | −38.884 | 1.00 | 61.49 | N |
| ATOM | 1829 | CA | LEU | A | 237 | 40.749 | −78.060 | −40.061 | 1.00 | 64.49 | C |
| ATOM | 1830 | CB | LEU | A | 237 | 40.584 | −76.741 | −40.825 | 1.00 | 64.59 | C |
| ATOM | 1831 | CG | LEU | A | 237 | 39.364 | −76.478 | −41.718 | 1.00 | 63.64 | C |
| ATOM | 1832 | CD1 | LEU | A | 237 | 38.040 | −76.818 | −41.057 | 1.00 | 66.77 | C |
| ATOM | 1833 | CD2 | LEU | A | 237 | 39.361 | −75.023 | −42.153 | 1.00 | 64.73 | C |
| ATOM | 1834 | C | LEU | A | 237 | 42.204 | −78.260 | −39.594 | 1.00 | 72.35 | C |
| ATOM | 1835 | O | LEU | A | 237 | 42.465 | −78.404 | −38.390 | 1.00 | 66.20 | O |
| ATOM | 1836 | N | THR | A | 238 | 43.139 | −78.276 | −40.543 | 1.00 | 77.72 | N |
| ATOM | 1837 | CA | THR | A | 238 | 44.566 | −78.443 | −40.242 | 1.00 | 79.47 | C |
| ATOM | 1838 | CB | THR | A | 238 | 45.110 | −79.730 | −40.886 | 1.00 | 76.25 | C |
| ATOM | 1839 | OG1 | THR | A | 238 | 44.037 | −80.665 | −41.079 | 1.00 | 74.86 | O |
| ATOM | 1840 | CG2 | THR | A | 238 | 46.190 | −80.347 | −40.010 | 1.00 | 71.93 | C |
| ATOM | 1841 | C | THR | A | 238 | 45.375 | −77.251 | −40.763 | 1.00 | 79.21 | C |
| ATOM | 1842 | O | THR | A | 238 | 45.183 | −76.820 | −41.900 | 1.00 | 82.33 | O |
| ATOM | 1843 | N | ASN | A | 239 | 46.269 | −76.707 | −39.943 | 1.00 | 80.05 | N |
| ATOM | 1844 | CA | ASN | A | 239 | 47.133 | −75.625 | −40.420 | 1.00 | 84.03 | C |
| ATOM | 1845 | CB | ASN | A | 239 | 47.415 | −74.567 | −39.324 | 1.00 | 79.55 | C |
| ATOM | 1846 | CG | ASN | A | 239 | 48.361 | −75.054 | −38.228 | 1.00 | 80.65 | C |
| ATOM | 1847 | OD1 | ASN | A | 239 | 48.606 | −76.251 | −38.076 | 1.00 | 80.17 | O |
| ATOM | 1848 | ND2 | ASN | A | 239 | 48.889 | −74.110 | −37.445 | 1.00 | 76.68 | N |
| ATOM | 1849 | C | ASN | A | 239 | 43.406 | −76.152 | −41.107 | 1.00 | 92.95 | C |
| ATOM | 1850 | O | ASN | A | 239 | 48.526 | −77.359 | −41.372 | 1.00 | 88.12 | O |
| ATOM | 1851 | N | LYS | A | 240 | 49.332 | −75.244 | −41.415 | 1.00 | 102.59 | N |
| ATOM | 1852 | CA | LYS | A | 240 | 50.606 | −75.599 | −42.045 | 1.00 | 103.12 | C |
| ATOM | 1853 | CB | LYS | A | 240 | 51.452 | −74.342 | −42.326 | 1.00 | 113.09 | C |
| ATOM | 1854 | CG | LYS | A | 240 | 51.724 | −73.444 | −41.117 | 1.00 | 121.32 | C |
| ATOM | 1855 | CD | LYS | A | 240 | 51.949 | −71.985 | −41.503 | 1.00 | 121.07 | C |
| ATOM | 1856 | CE | LYS | A | 240 | 50.639 | −71.207 | −41.554 | 1.00 | 123.36 | C |
| ATOM | 1857 | NZ | LYS | A | 240 | 50.864 | −69.737 | −41.488 | 1.00 | 107.84 | N |
| ATOM | 1858 | C | LYS | A | 240 | 51.378 | −76.638 | −41.224 | 1.00 | 98.30 | C |
| ATOM | 1859 | O | LYS | A | 240 | 51.745 | −77.687 | −41.743 | 1.00 | 101.69 | O |
| ATOM | 1860 | N | LYS | A | 241 | 51.579 | −76.361 | −39.939 | 1.00 | 94.24 | N |
| ATOM | 1861 | CA | LYS | A | 241 | 52.335 | −77.250 | −39.054 | 1.00 | 94.93 | C |
| ATOM | 1862 | CB | LYS | A | 241 | 52.982 | −76.453 | −37.910 | 1.00 | 96.09 | C |
| ATOM | 1863 | CG | LYS | A | 241 | 53.991 | −75.401 | −38.355 | 1.00 | 98.55 | C |
| ATOM | 1864 | CD | LYS | A | 241 | 54.717 | −74.777 | −37.168 | 1.00 | 105.95 | C |
| ATOM | 1865 | CE | LYS | A | 241 | 53.915 | −73.665 | −36.505 | 1.00 | 107.69 | C |
| ATOM | 1866 | NZ | LYS | A | 241 | 53.892 | −72.421 | −37.325 | 1.00 | 110.41 | N |
| ATOM | 1867 | C | LYS | A | 241 | 51.489 | −78.401 | −38.492 | 1.00 | 95.15 | C |
| ATOM | 1888 | O | LYS | A | 241 | 51.848 | −78.996 | −37.472 | 1.00 | 93.09 | O |
| ATOM | 1899 | N | GLY | A | 242 | 50.366 | −78.694 | −39.152 | 1.00 | 94.82 | N |
| ATOM | 1870 | CA | GLY | A | 242 | 49.507 | −79.837 | −38.811 | 1.00 | 92.08 | C |
| ATOM | 1871 | C | GLY | A | 242 | 48.680 | −79.813 | −37.526 | 1.00 | 86.38 | C |
| ATOM | 1872 | O | GLY | A | 242 | 48.005 | −80.800 | −37.213 | 1.00 | 79.57 | O |
| ATOM | 1873 | N | PHE | A | 243 | 48.741 | −78.709 | −36.780 | 1.00 | 88.07 | N |
| ATOM | 1874 | CA | PHE | A | 243 | 47.908 | −78.505 | −35.583 | 1.00 | 91.03 | C |
| ATOM | 1875 | CB | PHE | A | 243 | 48.341 | −77.228 | −34.840 | 1.00 | 106.28 | C |
| ATOM | 1876 | CG | PHE | A | 243 | 49.432 | −77.436 | −33.810 | 1.00 | 119.36 | C |
| ATOM | 1877 | CD1 | PHE | A | 243 | 49.146 | −78.009 | −37.566 | 1.00 | 175.59 | C |
| ATOM | 1878 | CE1 | PHE | A | 243 | 50.142 | −78.187 | −31.612 | 1.00 | 124.27 | C |
| ATOM | 1879 | CZ | PHE | A | 243 | 51.437 | −77.774 | −31.885 | 1.00 | 124.84 | C |
| ATOM | 1880 | CE2 | PHE | A | 243 | 51.734 | −77.286 | −33.109 | 1.00 | 124.57 | C |
| ATOM | 1881 | CD2 | PHE | A | 243 | 50.737 | −77.014 | −34.062 | 1.00 | 122.67 | C |
| ATOM | 1882 | C | PHE | A | 243 | 46.410 | −78.405 | −35.948 | 1.00 | 83.61 | C |
| ATOM | 1883 | O | PHE | A | 243 | 46.071 | −77.963 | −37.058 | 1.00 | 79.88 | O |
| ATOM | 1884 | N | PRO | A | 244 | 45.506 | −78.840 | −35.034 | 1.00 | 76.70 | N |
| ATOM | 1885 | CA | PRO | A | 244 | 44.069 | −78.592 | −35.254 | 1.00 | 68.42 | C |
| ATOM | 1886 | CB | PRO | A | 244 | 43.387 | −79.528 | −34.247 | 1.00 | 66.30 | C |
| ATOM | 1887 | CG | PRO | A | 244 | 44.424 | −79.834 | −33.222 | 1.00 | 65.69 | C |
| ATOM | 1888 | CD | PRO | A | 244 | 45.728 | −79.322 | −33.954 | 1.00 | 71.90 | C |
| ATOM | 1889 | C | PRO | A | 244 | 43.658 | −77.128 | −35.014 | 1.00 | 61.59 | C |
| ATOM | 1890 | O | PRO | A | 244 | 44.077 | −76.527 | −34.022 | 1.00 | 60.97 | O |
| ATOM | 1891 | N | VAL | A | 245 | 42.873 | −76.373 | −35.944 | 1.00 | 56.09 | N |
| ATOM | 1892 | CA | VAL | A | 245 | 42.312 | −75.209 | −35.863 | 1.00 | 55.09 | C |
| ATOM | 1893 | CB | VAL | A | 245 | 43.167 | −74.256 | −36.637 | 1.00 | 56.09 | C |
| ATOM | 1894 | CG1 | VAL | A | 245 | 44.547 | −73.989 | −36.019 | 1.00 | 56.45 | C |
| ATOM | 1895 | CG2 | VAL | A | 245 | 43.276 | −74.487 | −38.124 | 1.00 | 49.87 | C |
| ATOM | 1896 | C | VAL | A | 245 | 40.870 | −75.209 | −36.415 | 1.00 | 56.93 | C |
| ATOM | 1897 | O | VAL | A | 245 | 40.353 | −76.264 | −36.792 | 1.00 | 54.40 | O |
| ATOM | 1898 | N | LEU | A | 246 | 40.241 | −74.030 | −36.480 | 1.00 | 58.58 | N |
| ATOM | 1899 | CA | LEU | A | 246 | 38.844 | −73.886 | −36.943 | 1.00 | 55.94 | C |
| ATOM | 1900 | CB | LEU | A | 246 | 37.913 | −73.603 | −35.754 | 1.00 | 54.60 | C |
| ATOM | 1901 | CG | LEU | A | 246 | 37.653 | −74.679 | −34.698 | 1.00 | 53.24 | C |
| ATOM | 1902 | CD1 | LEU | A | 246 | 37.079 | −74.076 | −33.415 | 1.00 | 47.55 | C |
| ATOM | 1903 | CD2 | LEU | A | 246 | 36.753 | −75.760 | −35.268 | 1.00 | 51.11 | C |
| ATOM | 1904 | C | LEU | A | 246 | 38.641 | −72.776 | −37.988 | 1.00 | 56.27 | C |
| ATOM | 1905 | O | LEU | A | 246 | 39.333 | −71.753 | −37.961 | 1.00 | 62.54 | O |
| ATOM | 1906 | N | SER | A | 247 | 37.675 | −72.973 | −38.882 | 1.00 | 52.73 | N |
| ATOM | 1907 | CA | SER | A | 247 | 37.282 | −71.962 | −39.869 | 1.00 | 54.33 | C |
| ATOM | 1908 | CB | SER | A | 247 | 36.042 | −72.432 | −40.625 | 1.00 | 54.28 | C |
| ATOM | 1909 | OG | SER | A | 247 | 34.872 | −71.909 | −40.023 | 1.00 | 52.78 | O |
| ATOM | 1910 | C | SER | A | 247 | 36.941 | −70.641 | −39.201 | 1.00 | 54.58 | C |
| ATOM | 1911 | O | SER | A | 247 | 36.460 | −70.634 | −38.067 | 1.00 | 54.41 | O |
| ATOM | 1912 | N | LYS | A | 248 | 37.156 | −69.534 | −39.911 | 1.00 | 98.75 | N |
| ATOM | 1913 | CA | LYS | A | 248 | 36.830 | −68.195 | −39.384 | 1.00 | 66.78 | C |
| ATOM | 1914 | CB | LYS | A | 248 | 36.929 | −67.105 | −40.475 | 1.00 | 69.86 | C |
| ATOM | 1915 | CG | LYS | A | 248 | 38.296 | −66.920 | −41.139 | 1.00 | 73.70 | C |
| ATOM | 1916 | CD | LYS | A | 248 | 39.400 | −66.505 | −40.167 | 1.00 | 76.63 | C |
| ATOM | 1917 | CE | LYS | A | 248 | 39.411 | −65.006 | −39.880 | 1.00 | 81.27 | C |
| ATOM | 1918 | NZ | LYS | A | 248 | 40.475 | −64.638 | −38.897 | 1.00 | 74.54 | N |
| ATOM | 1919 | C | LYS | A | 248 | 35.433 | −68.171 | −38.750 | 1.00 | 66.96 | C |
| ATOM | 1920 | G | LYS | A | 248 | 35.255 | −67.690 | −37.677 | 1.00 | 67.39 | O |
| ATOM | 1921 | N | MET | A | 249 | 34.455 | −68.711 | −39.476 | 1.00 | 63.97 | N |
| ATOM | 1922 | CA | MET | A | 249 | 33.080 | −68.780 | −39.007 | 1.00 | 62.96 | C |
| ATOM | 1923 | CB | MET | A | 249 | 32.174 | −69.451 | −40.050 | 1.00 | 71.00 | C |
| ATOM | 1924 | CG | MET | A | 249 | 31.923 | −68.593 | −41.295 | 1.00 | 84.64 | C |
| ATOM | 1925 | SD | MET | A | 249 | 31.265 | −66.913 | −41.023 | 1.00 | 97.53 | S |
| ATOM | 1926 | CE | MET | A | 249 | 32.724 | −65.919 | −40.655 | 1.00 | 86.77 | C |
| ATOM | 1927 | C | MET | A | 249 | 33.964 | −69.448 | −37.647 | 1.00 | 57.77 | C |
| ATOM | 1928 | G | MET | A | 249 | 32.474 | −68.835 | −36.706 | 1.00 | 60.03 | O |
| ATOM | 1929 | N | HIS | A | 250 | 33.436 | −70.688 | −37.547 | 1.00 | 34.08 | N |
| ATOM | 1930 | CA | HIS | A | 250 | 33.481 | −71.404 | −36.282 | 1.00 | 48.57 | C |
| ATOM | 1931 | CB | HIS | A | 250 | 34.169 | −72.746 | −36.447 | 1.00 | 44.66 | C |
| ATOM | 1932 | CG | HIS | A | 250 | 33.371 | −73.754 | −37.217 | 1.00 | 42.87 | C |
| ATOM | 1933 | ND1 | HIS | A | 250 | 33.212 | −73.692 | −38.586 | 1.00 | 41.98 | N |
| ATOM | 1934 | CE1 | HIS | A | 250 | 32.490 | −74.724 | −38.992 | 1.00 | 41.68 | C |
| ATOM | 1935 | NE2 | HIS | A | 250 | 32.173 | −75.455 | −37.936 | 1.00 | 41.32 | N |
| ATOM | 1936 | CD2 | HIS | A | 250 | 32.716 | −74.872 | −36.814 | 1.00 | 41.61 | C |
| ATOM | 1937 | C | HIS | A | 250 | 34.159 | −70.612 | −35.166 | 1.00 | 53.44 | C |
| ATOM | 1938 | O | HIS | A | 250 | 33.746 | −70.710 | −34.016 | 1.00 | 62.63 | O |
| ATOM | 1939 | N | GLN | A | 251 | 35.179 | −69.819 | −35.484 | 1.00 | 53.86 | N |
| ATOM | 1940 | CA | GLN | A | 251 | 35.806 | −68.986 | −34.457 | 1.00 | 55.47 | C |
| ATOM | 1941 | CB | GLN | A | 251 | 37.082 | −68.313 | −34.962 | 1.00 | 60.37 | C |
| ATOM | 1942 | CG | GLN | A | 251 | 38.101 | −69.237 | −35.628 | 1.00 | 65.30 | C |
| ATOM | 1943 | CD | GLN | A | 251 | 39.355 | −68.490 | −36.060 | 1.00 | 64.59 | C |
| ATOM | 1044 | OC1 | GLN | A | 251 | 40.197 | −68.152 | −35.234 | 1.00 | 65.90 | O |
| ATOM | 1945 | NE2 | GLN | A | 251 | 39.475 | −68.219 | −37.354 | 1.00 | 64.44 | N |
| ATOM | 1946 | C | GLN | A | 251 | 34.819 | −67.933 | −33.950 | 1.00 | 57.71 | C |
| ATOM | 1947 | O | GLN | A | 251 | 34.626 | −67.789 | −32.732 | 1.00 | 58.12 | O |
| ATOM | 1948 | N | ARG | A | 252 | 34.175 | −67.222 | −34.879 | 1.00 | 58.59 | N |
| ATOM | 1949 | CA | ARG | A | 252 | 33.119 | −66.263 | −34.522 | 1.00 | 59.00 | C |
| ATOM | 1950 | CB | ARG | A | 252 | 32.366 | −65.745 | −35.759 | 1.00 | 66.80 | C |
| ATOM | 1951 | CG | ARG | A | 252 | 32.940 | −64.466 | −36.379 | 1.00 | 76.35 | C |
| ATOM | 1952 | CD | ARG | A | 252 | 32.103 | −63.936 | −37.547 | 1.00 | 84.79 | C |
| ATOM | 1953 | NE | ARG | A | 252 | 30.701 | −63.679 | −37.180 | 1.00 | 92.34 | N |
| ATOM | 1954 | CZ | ARG | A | 252 | 29.668 | −63.672 | −38.031 | 1.00 | 93.57 | C |
| ATOM | 1955 | NH1 | ARG | A | 252 | 29.841 | −63.908 | −39.331 | 1.00 | 89.36 | N |
| ATOM | 1956 | NH2 | ARG | A | 252 | 28.444 | −63.436 | −37.574 | 1.00 | 91.23 | N |
| ATOM | 1957 | C | ARG | A | 252 | 32.152 | −66.878 | −33.515 | 1.00 | 55.53 | C |
| ATOM | 1958 | O | ARG | A | 252 | 31.893 | −66.298 | −32.458 | 1.00 | 58.09 | O |
| ATOM | 1959 | N | LEU | A | 253 | 31.650 | −68.069 | −33.826 | 1.00 | 51.71 | N |
| ATOM | 1960 | CA | LEU | A | 253 | 30.688 | −68.734 | −32.953 | 1.00 | 50.86 | C |
| ATOM | 1961 | CB | LEU | A | 253 | 30.111 | −69.993 | −33.610 | 1.00 | 48.22 | C |
| ATOM | 1962 | CG | LEU | A | 253 | 29.133 | −70.876 | −32.805 | 1.00 | 47.32 | C |
| ATOM | 1963 | CD1 | LEU | A | 253 | 27.955 | −70.074 | −32.275 | 1.00 | 46.75 | C |
| ATOM | 1964 | CD2 | LEU | A | 253 | 28.631 | −72.070 | −33.601 | 1.00 | 41.03 | C |
| ATOM | 1965 | C | LEU | A | 253 | 31.301 | −69.052 | −31.597 | 1.00 | 52.12 | C |
| ATOM | 1966 | O | LEU | A | 253 | 30.662 | −68.856 | −30.567 | 1.00 | 55.71 | O |
| ATOM | 1967 | N | ILE | A | 254 | 32.543 | −69.521 | −31.594 | 1.00 | 52.88 | N |
| ATOM | 1968 | CA | ILE | A | 254 | 33.212 | −69.832 | −30.343 | 1.00 | 51.47 | C |
| ATOM | 1969 | CB | ILE | A | 254 | 34.630 | −70.413 | −30.554 | 1.00 | 49.65 | C |
| ATOM | 1970 | CG1 | ILE | A | 254 | 34.574 | −71.703 | −31.382 | 1.00 | 46.18 | C |
| ATOM | 1971 | CD1 | ILE | A | 254 | 33.874 | −72.862 | −30.716 | 1.00 | 45.31 | C |
| ATOM | 1972 | CG2 | ILE | A | 254 | 35.327 | −70.637 | −29.216 | 1.00 | 45.02 | C |
| ATOM | 1973 | C | ILE | A | 254 | 33.274 | −68.572 | −29.502 | 1.00 | 50.70 | C |
| ATOM | 1974 | O | ILE | A | 254 | 32.914 | −68.588 | −28.320 | 1.00 | 47.68 | O |
| ATOM | 1975 | N | PHE | A | 255 | 33.706 | −67.480 | −30.127 | 1.00 | 55.22 | N |
| ATOM | 1976 | CA | PHE | A | 255 | 33.793 | −66.181 | −29.440 | 1.00 | 59.92 | C |
| ATOM | 1977 | CB | PHE | A | 255 | 34.346 | −65.095 | −30.372 | 1.00 | 64.40 | C |
| ATOM | 1978 | CG | PHE | A | 255 | 35.829 | −65.212 | −30.640 | 1.00 | 71.44 | C |
| ATOM | 1979 | CD1 | PHE | A | 255 | 36.759 | −65.092 | −29.601 | 1.00 | 78.19 | C |
| ATOM | 1980 | CE1 | PHE | A | 255 | 38.124 | −65.192 | −29.850 | 1.00 | 77.35 | C |
| ATOM | 1981 | CZ | PHE | A | 255 | 38.579 | −65.401 | −31.145 | 1.00 | 73.21 | C |
| ATOM | 1982 | CE2 | PHE | A | 255 | 37.669 | −65.513 | −32.187 | 1.00 | 73.49 | C |
| ATOM | 1983 | CD2 | PHE | A | 255 | 35.304 | −65.414 | −31.935 | 1.00 | 72.11 | C |
| ATOM | 1984 | C | PHE | A | 255 | 32.454 | −65.750 | −28.816 | 1.00 | 54.79 | C |
| ATOM | 1985 | O | PHE | A | 255 | 32.423 | −65.766 | −27.678 | 1.00 | 51.84 | O |
| ATOM | 1986 | N | ARG | A | 256 | 31.360 | −65.953 | −29.551 | 1.00 | 51.90 | N |
| ATOM | 1987 | CA | ARG | A | 256 | 30.016 | −65.719 | −29.015 | 1.00 | 54.39 | C |
| ATOM | 1988 | CB | ARG | A | 256 | 28.940 | −65.943 | −30.088 | 1.00 | 59.07 | C |
| ATOM | 1989 | CG | ARG | A | 256 | 28.816 | −64.838 | −31.139 | 1.00 | 64.35 | C |
| ATOM | 1990 | CD | ARG | A | 256 | 27.718 | −63.843 | −30.765 | 1.00 | 67.70 | C |
| ATOM | 1991 | NE | ARG | A | 256 | 26.801 | −63.577 | −31.886 | 1.00 | 70.43 | N |
| ATOM | 1992 | CZ | ARG | A | 256 | 25.802 | −64.376 | −32.276 | 1.00 | 68.04 | C |
| ATOM | 1993 | NH1 | ARG | A | 256 | 25.554 | −65.533 | −31.664 | 1.00 | 67.23 | N |
| ATOM | 1994 | NH2 | ARG | A | 256 | 25.046 | −64.922 | −33.298 | 1.00 | 68.29 | N |
| ATOM | 1995 | C | ARG | A | 256 | 29.766 | −66.618 | −27.801 | 1.00 | 52.37 | C |
| ATOM | 1996 | O | ARG | A | 256 | 29.310 | −66.153 | −26.752 | 1.00 | 55.21 | O |
| ATOM | 1997 | N | LEU | A | 257 | 30.103 | −67.897 | −27.930 | 1.00 | 46.30 | N |
| ATOM | 1998 | CA | LEU | A | 257 | 29.928 | −68.833 | −26.829 | 1.00 | 43.47 | C |
| ATOM | 1999 | CB | LEU | A | 257 | 30.114 | −70.264 | −27.323 | 1.00 | 44.63 | C |
| ATOM | 2000 | CG | LEU | A | 257 | 29.050 | −70.776 | −28.316 | 1.00 | 46.23 | C |
| ATOM | 2001 | CD1 | LEU | A | 257 | 29.645 | −71.849 | −29.216 | 1.00 | 43.01 | C |
| ATOM | 2002 | CD2 | LEU | A | 257 | 27.820 | −71.328 | −27.604 | 1.00 | 40.84 | C |
| ATOM | 2003 | C | LEU | A | 257 | 30.844 | −68.530 | −25.633 | 1.00 | 44.37 | C |
| ATOM | 2004 | O | LEU | A | 257 | 30.468 | −68.765 | −24.488 | 1.00 | 44.65 | O |
| ATOM | 2005 | N | LEU | A | 258 | 32.035 | −67.990 | −25.875 | 1.00 | 44.69 | N |
| ATOM | 2006 | CA | LEU | A | 258 | 32.894 | −67.545 | −24.744 | 1.00 | 47.95 | C |
| ATOM | 2007 | CB | LEU | A | 258 | 34.307 | −67.248 | −25.179 | 1.00 | 43.95 | C |
| ATOM | 2008 | CG | LEU | A | 258 | 35.127 | −68.455 | −25.636 | 1.00 | 46.75 | C |
| ATOM | 2009 | CD1 | LEU | A | 258 | 36.183 | −68.949 | −26.655 | 1.00 | 53.34 | C |
| ATOM | 2010 | CD2 | LEU | A | 258 | 35.758 | −69.285 | −24.454 | 1.00 | 48.78 | C |
| ATOM | 2011 | C | LEU | A | 258 | 32.240 | −66.553 | −23.924 | 1.00 | 49.66 | C |
| ATOM | 2012 | O | LEU | A | 258 | 32.214 | −66.540 | −22.697 | 1.00 | 50.36 | O |
| ATOM | 2013 | N | LYS | A | 259 | 31.695 | −65.545 | −24.615 | 1.00 | 56.08 | N |
| ATOM | 2014 | CA | LYS | A | 259 | 30.928 | −64.455 | −23.990 | 1.00 | 57.96 | C |
| ATOM | 2015 | CB | LYS | A | 259 | 30.399 | −63.495 | −25.058 | 1.00 | 63.14 | C |
| ATOM | 2016 | CG | LYS | A | 259 | 31.166 | −62.283 | −25.170 | 1.00 | 68.74 | C |
| ATOM | 2017 | CD | LYS | A | 259 | 30.502 | −61.679 | −24.346 | 1.00 | 80.95 | C |
| ATOM | 2018 | CE | LYS | A | 259 | 31.487 | −60.291 | −23.481 | 1.00 | 87.05 | C |
| ATOM | 2019 | NZ | LYS | A | 259 | 32.560 | −59.503 | −24.257 | 1.00 | 89.35 | N |
| ATOM | 2020 | C | LYS | A | 259 | 29.786 | −64.959 | −23.096 | 1.00 | 60.25 | C |
| ATOM | 2021 | O | LYS | A | 259 | 29.467 | −64.337 | −22.099 | 1.00 | 68.02 | O |
| ATOM | 2022 | N | LEU | A | 260 | 29.205 | −66.205 | −23.442 | 1.00 | 58.51 | N |
| ATOM | 2023 | CA | LEU | A | 260 | 28.161 | −66.734 | −22.650 | 1.00 | 57.27 | C |
| ATOM | 2024 | CB | LEU | A | 260 | 27.200 | −67.444 | −23.603 | 1.00 | 56.48 | C |
| ATOM | 2025 | CG | LEU | A | 260 | 25.746 | −67.527 | −23.169 | 1.00 | 55.15 | C |
| ATOM | 2026 | CD1 | LEU | A | 260 | 25.145 | −66.239 | −23.067 | 1.00 | 54.38 | C |
| ATOM | 2027 | CD2 | LEU | A | 260 | 24.960 | −68.867 | −24.160 | 1.00 | 57.06 | C |
| ATOM | 2028 | C | LEU | A | 260 | 28.724 | −67.740 | −21.641 | 1.00 | 58.98 | C |
| ATOM | 2029 | O | LEU | A | 260 | 27.991 | −68.396 | −20.883 | 1.00 | 56.48 | O |
| ATOM | 2030 | N | GLU | A | 261 | 30.037 | −67.883 | −21.641 | 1.00 | 56.45 | N |
| ATOM | 2031 | CA | GLU | A | 261 | 30.693 | −68.750 | −20.670 | 1.00 | 56.32 | C |
| ATOM | 2032 | CB | GLU | A | 261 | 30.749 | −68.103 | −19.285 | 1.00 | 57.14 | C |
| ATOM | 2033 | CG | GLU | A | 261 | 30.937 | −66.596 | −19.281 | 1.00 | 65.83 | C |
| ATOM | 2034 | CD | GLU | A | 261 | 30.986 | −66.031 | −17.871 | 1.00 | 75.64 | C |
| ATOM | 2035 | OE1 | GLU | A | 261 | 30.815 | −64.799 | −17.702 | 1.00 | 80.95 | O |
| ATOM | 2036 | OE2 | GLU | A | 261 | 31.197 | −66.175 | −16.926 | 1.00 | 73.20 | O |
| ATOM | 2037 | C | GLU | A | 261 | 30.043 | −70.225 | −20.567 | 1.00 | 50.82 | C |
| ATOM | 2038 | O | GLU | A | 261 | 29.661 | −70.557 | −19.495 | 1.00 | 63.15 | O |
| ATOM | 2039 | N | VAL | A | 262 | 29.942 | −70.820 | −21.683 | 1.00 | 45.13 | N |
| ATOM | 2040 | CA | VAL | A | 262 | 29.568 | −72.210 | −21.664 | 1.00 | 42.96 | C |
| ATOM | 2041 | CB | VAL | A | 262 | 78.969 | −72.639 | −23.009 | 1.00 | 42.98 | C |
| ATOM | 2042 | CG1 | VAL | A | 262 | 27.669 | −71.906 | −23.281 | 1.00 | 39.27 | C |
| ATOM | 2043 | CG2 | VAL | A | 262 | 29.960 | −72.389 | −24.134 | 1.00 | 47.78 | C |
| ATOM | 2044 | C | VAL | A | 262 | 30.812 | −73.051 | −21.396 | 1.00 | 47.75 | C |
| ATOM | 2045 | O | VAL | A | 262 | 31.944 | −72.548 | −21.453 | 1.00 | 44.51 | O |
| ATOM | 2046 | N | GLN | A | 263 | 30.601 | −74.331 | −21.114 | 1.00 | 45.31 | N |
| ATOM | 2047 | CA | GLN | A | 263 | 31.705 | −75.267 | −21.055 | 1.00 | 47.58 | C |
| ATOM | 2048 | CB | GLN | A | 263 | 31.483 | −76.276 | −19.936 | 1.00 | 46.98 | C |
| ATOM | 2049 | CG | GLN | A | 263 | 31.722 | −75.694 | −18.545 | 1.00 | 45.76 | C |
| ATOM | 2050 | CD | GLN | A | 263 | 31.444 | −76.710 | −17.469 | 1.00 | 47.32 | C |
| ATOM | 2051 | CE1 | GLN | A | 263 | 30.325 | −77.210 | −17.347 | 1.00 | 48.21 | O |
| ATOM | 2052 | NE2 | GLN | A | 263 | 32.462 | −77.040 | −16.693 | 1.00 | 47.27 | N |
| ATOM | 2053 | C | GLN | A | 263 | 31.844 | −75.971 | −22.402 | 1.00 | 51.28 | C |
| ATOM | 2054 | O | GLN | A | 263 | 30.842 | −76.284 | −23.051 | 1.00 | 53.48 | O |
| ATOM | 2055 | N | PHE | A | 264 | 33.084 | −76.209 | −22.831 | 1.00 | 53.11 | N |
| ATOM | 2056 | CA | PHE | A | 764 | 33.321 | −76.851 | −24.121 | 1.00 | 49.88 | C |
| ATOM | 2057 | CB | PHE | A | 264 | 34.412 | −76.140 | −24.891 | 1.00 | 46.04 | C |
| ATOM | 2058 | CG | PHE | A | 264 | 34.004 | −74.769 | −25.350 | 1.00 | 44.76 | C |
| ATOM | 2059 | CD1 | PHE | A | 264 | 33.391 | −74.593 | −26.588 | 1.00 | 40.62 | C |
| ATOM | 2060 | CE1 | PHE | A | 264 | 33.007 | −73.346 | −27.005 | 1.00 | 37.92 | C |
| ATOM | 2061 | CZ | PHE | A | 264 | 33.202 | −72.250 | −26.183 | 1.00 | 38.19 | C |
| ATOM | 2062 | CE2 | PHE | A | 264 | 33.791 | −77.403 | −74.946 | 1.00 | 40.47 | C |
| ATOM | 2063 | CD2 | PHE | A | 264 | 34.190 | −73.661 | −24.529 | 1.00 | 43.13 | C |
| ATOM | 2064 | C | PHE | A | 264 | 33.617 | −78.316 | −23.976 | 1.00 | 51.86 | C |
| ATOM | 2065 | O | PHE | A | 264 | 34.261 | −78.729 | −23.022 | 1.00 | 54.60 | O |
| ATOM | 2066 | N | ILE | A | 265 | 33.102 | −79.102 | −24.915 | 1.00 | 49.99 | N |
| ATOM | 2067 | CA | ILE | A | 265 | 33.203 | −80.539 | −24.839 | 1.00 | 49.20 | C |
| ATOM | 2068 | CB | ILE | A | 265 | 31.844 | −81.204 | −24.524 | 1.00 | 49.05 | C |
| ATOM | 2069 | CG1 | ILE | A | 265 | 31.328 | −80.837 | −23.116 | 1.00 | 48.44 | C |
| ATOM | 2070 | CD1 | ILE | A | 265 | 29.905 | −81.556 | −22.841 | 1.00 | 42.10 | C |
| ATOM | 2071 | CG2 | ILE | A | 265 | 31.930 | −82.718 | −24.706 | 1.00 | 47.98 | C |
| ATOM | 2072 | C | ILE | A | 265 | 33.693 | −81.064 | −26.173 | 1.00 | 49.47 | C |
| ATOM | 2073 | O | ILE | A | 265 | 33.062 | −80.826 | −27.201 | 1.00 | 49.39 | O |
| ATOM | 2074 | N | ILE | A | 266 | 34.805 | −81.802 | −26.132 | 1.00 | 48.25 | N |
| ATOM | 2075 | CA | ILE | A | 266 | 35.367 | −82.450 | −27.304 | 1.00 | 43.97 | C |
| ATOM | 2076 | CB | ILE | A | 266 | 36.914 | −82.467 | −27.276 | 1.00 | 42.22 | C |
| ATOM | 2077 | CG1 | ILE | A | 266 | 37.495 | −81.116 | −26.814 | 1.00 | 40.96 | C |
| ATOM | 2078 | CD1 | ILE | A | 266 | 37.641 | −80.055 | −27.876 | 1.00 | 41.73 | C |
| ATOM | 2079 | CG2 | ILE | A | 266 | 37.466 | −82.934 | −28.621 | 1.00 | 42.32 | C |
| ATOM | 2080 | C | ILE | A | 266 | 34.886 | −83.896 | −27.403 | 1.00 | 43.49 | C |
| ATOM | 2081 | O | ILE | A | 266 | 35.102 | −84.705 | −26.485 | 1.00 | 41.43 | O |
| ATOM | 2082 | N | THR | A | 267 | 34.282 | −84.224 | −28.546 | 1.00 | 39.69 | N |
| ATOM | 2083 | CA | THR | A | 267 | 33.771 | −85.569 | −28.792 | 1.00 | 41.29 | C |
| ATOM | 2084 | CB | THR | A | 267 | 32.231 | −85.529 | −28.939 | 1.00 | 36.46 | C |
| ATOM | 2085 | OG1 | THR | A | 267 | 31.723 | −86.850 | −29.093 | 1.00 | 40.34 | O |
| ATOM | 2086 | CG2 | THR | A | 267 | 31.802 | −84.705 | −30.141 | 1.00 | 33.93 | C |
| ATOM | 2087 | C | THR | A | 267 | 34.466 | −86.190 | −30.027 | 1.00 | 42.85 | C |
| ATOM | 2088 | O | THR | A | 267 | 34.950 | −85.456 | −30.875 | 1.00 | 42.40 | O |
| ATOM | 2089 | N | GLY | A | 268 | 34.512 | −87.521 | −30.121 | 1.00 | 46.41 | N |
| ATOM | 2090 | CA | GLY | A | 268 | 35.145 | −88.204 | −31.276 | 1.00 | 53.98 | C |
| ATOM | 2091 | C | GLY | A | 268 | 36.619 | −88.577 | −31.090 | 1.00 | 59.97 | C |
| ATOM | 2092 | O | GLY | A | 268 | 37.239 | −88.152 | −30.110 | 1.00 | 59.79 | O |
| ATOM | 2093 | N | THR | A | 269 | 37.191 | −89.365 | −32.012 | 1.00 | 60.76 | N |
| ATOM | 2094 | CA | THR | A | 269 | 38.618 | −89.770 | −31.902 | 1.00 | 60.88 | C |
| ATOM | 2095 | CB | THR | A | 269 | 36.962 | −91.097 | −32.628 | 1.00 | 59.65 | C |
| ATOM | 2096 | OG1 | THR | A | 269 | 38.361 | −91.114 | −33.932 | 1.00 | 59.10 | O |
| ATOM | 2097 | CG2 | THR | A | 269 | 38.528 | −92.320 | −31.819 | 1.00 | 61.41 | C |
| ATOM | 2098 | C | THR | A | 269 | 39.563 | −83.708 | −32.444 | 1.00 | 62.31 | C |
| ATOM | 2099 | O | THR | A | 269 | 39.140 | −87.787 | −33.140 | 1.00 | 58.97 | O |
| ATOM | 2100 | N | ASN | A | 270 | 40.848 | −88.860 | −32.136 | 1.00 | 69.16 | N |
| ATOM | 2101 | CA | ASN | A | 270 | 41.882 | −87.991 | −32.686 | 1.00 | 79.51 | C |
| ATOM | 2102 | CB | ASN | A | 270 | 43.180 | −88.126 | −31.888 | 1.00 | 80.21 | C |
| ATOM | 2103 | CG | ASN | A | 270 | 44.271 | −87.213 | −32.400 | 1.00 | 84.75 | C |
| ATOM | 2104 | OD1 | ASN | A | 270 | 43.997 | −86.169 | −33.002 | 1.00 | 83.32 | O |
| ATOM | 2105 | ND2 | ASN | A | 270 | 45.519 | −87.600 | −32.168 | 1.00 | 91.73 | N |
| ATOM | 2106 | C | ASN | A | 270 | 42.142 | −88.281 | −34.168 | 1.00 | 84.50 | C |
| ATOM | 2107 | O | ASN | A | 270 | 42.468 | −89.411 | −34.536 | 1.00 | 86.44 | O |
| ATOM | 2108 | N | HIS | A | 271 | 41.992 | −87.259 | −35.009 | 1.00 | 86.51 | N |
| ATOM | 2109 | CA | HIS | A | 271 | 42.177 | −87.420 | −36.451 | 1.00 | 93.02 | C |
| ATOM | 2110 | CB | HIS | A | 271 | 40.954 | −86.918 | −37.223 | 1.00 | 92.02 | C |
| ATOM | 2111 | CG | HIS | A | 271 | 39.749 | −87.798 | −37.077 | 1.00 | 92.65 | C |
| ATOM | 2112 | ND1 | HIS | A | 271 | 38.486 | −87.300 | −36.842 | 1.00 | 88.60 | N |
| ATOM | 2113 | CE1 | HIS | A | 271 | 37.627 | −88.301 | −36.759 | 1.00 | 91.86 | C |
| ATOM | 2114 | NE2 | HIS | A | 271 | 38.289 | −89.433 | −36.920 | 1.00 | 96.54 | N |
| ATOM | 2115 | CD2 | HIS | A | 271 | 39.619 | −89.147 | −37.120 | 1.00 | 93.69 | C |
| ATOM | 2116 | C | HIS | A | 271 | 43.472 | −86.769 | −36.936 | 1.00 | 102.68 | C |
| ATOM | 2117 | O | HIS | A | 271 | 44.042 | −87.167 | −37.959 | 1.00 | 101.52 | O |
| ATOM | 2118 | N | HIS | A | 272 | 43.931 | −85.774 | −36.182 | 1.00 | 111.60 | N |
| ATOM | 2119 | CA | HIS | A | 272 | 45.265 | −85.211 | −36.348 | 1.00 | 120.65 | C |
| ATOM | 2120 | CB | HIS | A | 272 | 45.286 | −83.756 | −35.881 | 1.00 | 122.38 | C |
| ATOM | 2121 | CG | HIS | A | 272 | 44.264 | −82.896 | −36.556 | 1.00 | 126.93 | C |
| ATOM | 2122 | ND1 | HIS | A | 272 | 42.920 | −82.363 | −36.258 | 1.00 | 127.96 | N |
| ATOM | 2123 | CE1 | HIS | A | 272 | 42.260 | −82.098 | −37.007 | 1.00 | 130.33 | C |
| ATOM | 2124 | NE2 | HIS | A | 272 | 43.128 | −81.472 | −37.781 | 1.00 | 130.68 | N |
| ATOM | 2125 | CD2 | HIS | A | 272 | 44.388 | −81.955 | −37.521 | 1.00 | 130.62 | C |
| ATOM | 2126 | C | HIS | A | 272 | 46.220 | −86.076 | −35.529 | 1.00 | 125.42 | C |
| ATOM | 2127 | O | HIS | A | 272 | 46.579 | −85.735 | −34.396 | 1.00 | 131.06 | O |
| ATOM | 2128 | N | SER | A | 273 | 46.616 | −87.201 | −36.124 | 1.00 | 126.94 | N |
| ATOM | 2129 | CA | SER | A | 273 | 47.279 | −88.302 | −35.418 | 1.00 | 122.39 | C |
| ATOM | 2130 | CB | SER | A | 273 | 47.429 | −89.500 | −36.357 | 1.00 | 117.69 | C |
| ATOM | 2131 | OG | SER | A | 273 | 47.652 | −90.687 | −35.624 | 1.00 | 112.72 | O |
| ATOM | 2132 | C | SER | A | 273 | 48.630 | −87.956 | −34.773 | 1.00 | 123.01 | C |
| ATOM | 2133 | O | SER | A | 273 | 48.936 | −88.440 | −33.678 | 1.00 | 118.30 | O |
| ATOM | 2134 | N | GLU | A | 274 | 49.420 | −87.114 | −35.445 | 1.00 | 124.62 | N |
| ATOM | 2135 | CA | GLU | A | 274 | 50.750 | −86.701 | −34.954 | 1.00 | 120.75 | C |
| ATOM | 2136 | CB | GLU | A | 274 | 51.623 | −86.137 | −36.093 | 1.00 | 119.13 | C |
| ATOM | 2137 | CG | GLU | A | 274 | 51.742 | −87.007 | −37.347 | 1.00 | 113.17 | C |
| ATOM | 2138 | CD | GLU | A | 274 | 50.692 | −86.681 | −38.401 | 1.00 | 107.49 | C |
| ATOM | 2139 | OE1 | GLU | A | 274 | 50.053 | −87.619 | −38.920 | 1.00 | 101.93 | O |
| ATOM | 2140 | OE2 | GLU | A | 274 | 50.501 | −85.486 | −38.712 | 1.00 | 102.86 | O |
| ATOM | 2141 | C | GLU | A | 274 | 50.690 | −85.585 | −33.795 | 1.00 | 116.52 | C |
| ATOM | 2142 | O | GLU | A | 274 | 51.688 | −85.024 | −33.468 | 1.00 | 104.78 | O |
| ATOM | 2143 | N | LYS | A | 275 | 49.520 | −85.664 | −33.163 | 1.00 | 115.83 | N |
| ATOM | 2144 | CA | LYS | A | 275 | 49.312 | −84.642 | −32.044 | 1.00 | 113.94 | C |
| ATOM | 2145 | CB | LYS | A | 275 | 48.596 | −83.368 | −32.515 | 1.00 | 116.81 | C |
| ATOM | 2146 | CG | LYS | A | 275 | 49.251 | −82.614 | −33.671 | 1.00 | 121.05 | C |
| ATOM | 2147 | CD | LYS | A | 275 | 50.412 | −81.729 | −33.220 | 1.00 | 121.15 | C |
| ATOM | 2148 | CE | LYS | A | 275 | 50.792 | −80.697 | −34.277 | 1.00 | 112.20 | C |
| ATOM | 2149 | NZ | LYS | A | 275 | 51.179 | −81.296 | −35.585 | 1.00 | 105.56 | N |
| ATOM | 2150 | C | LYS | A | 275 | 48.493 | −85.300 | −30.931 | 1.00 | 112.38 | C |
| ATOM | 2151 | O | LYS | A | 275 | 47.734 | −86.143 | −31.174 | 1.00 | 108.58 | O |
| ATOM | 2152 | N | GLU | A | 276 | 48.654 | −84.790 | −29.713 | 1.00 | 109.30 | N |
| ATOM | 2153 | CA | GLU | A | 276 | 47.868 | −85.228 | −28.560 | 1.00 | 111.55 | C |
| ATOM | 2154 | CB | GLU | A | 276 | 48.602 | −84.134 | −27.263 | 1.00 | 114.89 | C |
| ATOM | 2155 | CG | GLU | A | 276 | 47.959 | −83.280 | −25.948 | 1.00 | 119.60 | C |
| ATOM | 2156 | CD | GLU | A | 276 | 48.137 | −86.762 | −25.650 | 1.00 | 119.68 | C |
| ATOM | 2157 | OE1 | GLU | A | 276 | 48.867 | −87.089 | −24.682 | 1.00 | 120.15 | O |
| ATOM | 2158 | OE2 | GLU | A | 276 | 47.543 | −87.600 | −26.371 | 1.00 | 110.35 | O |
| ATOM | 2159 | C | GLU | A | 276 | 46.448 | −84.627 | −28.608 | 1.00 | 104.92 | C |
| ATOM | 2160 | O | GLU | A | 276 | 46.214 | −83.636 | −29.298 | 1.00 | 98.05 | O |
| ATOM | 2161 | N | PHE | A | 277 | 45.508 | −85.139 | −27.887 | 1.00 | 98.94 | N |
| ATOM | 2162 | CA | PHE | A | 277 | 44.167 | −84.675 | −27.707 | 1.00 | 96.81 | C |
| ATOM | 2163 | CB | PHE | A | 277 | 43.303 | −85.376 | −26.821 | 1.00 | 98.90 | C |
| ATOM | 2164 | CG | PHE | A | 277 | 42.536 | −86.621 | −27.571 | 1.00 | 131.65 | C |
| ATOM | 2165 | CD1 | PHE | A | 277 | 42.887 | −87.972 | −27.463 | 1.00 | 197.66 | C |
| ATOM | 2166 | CE1 | PHE | A | 277 | 42.184 | −88.953 | −28.149 | 1.00 | 195.41 | C |
| ATOM | 2167 | CZ | PHE | A | 277 | 41.113 | −81.585 | −78.953 | 1.00 | 199.45 | C |
| ATOM | 2168 | CE2 | PHE | A | 277 | 40.750 | −87.241 | −29.067 | 1.00 | 103.53 | C |
| ATOM | 2169 | CD2 | PHE | A | 277 | 41.455 | −86.268 | −28.375 | 1.00 | 96.57 | C |
| ATOM | 2170 | C | PHE | A | 277 | 44.211 | −83.285 | −27.077 | 1.00 | 93.70 | C |
| ATOM | 2171 | O | PHE | A | 277 | 43.274 | −82.492 | −27.242 | 1.00 | 94.16 | O |
| ATOM | 2172 | N | CYS | A | 278 | 45.290 | −83.007 | −26.346 | 1.00 | 81.82 | N |
| ATOM | 2173 | CA | CYS | A | 278 | 45.469 | −81.723 | −25.684 | 1.00 | 74.52 | C |
| ATOM | 2174 | CB | CYS | A | 278 | 46.630 | −81.785 | −24.675 | 1.00 | 30.41 | C |
| ATOM | 2175 | SG | CYS | A | 278 | 48.308 | −81.701 | −25.354 | 1.00 | 83.20 | S |
| ATOM | 2176 | C | CYS | A | 278 | 45.656 | −80.572 | −26.686 | 1.00 | 69.31 | C |
| ATOM | 2177 | O | CYS | A | 278 | 45.564 | −79.407 | −26.317 | 1.00 | 68.96 | O |
| ATOM | 2178 | N | SER | A | 279 | 45.897 | −80.904 | −27.951 | 1.00 | 66.91 | N |
| ATOM | 2179 | CA | SER | A | 279 | 46.110 | −79.901 | −28.992 | 1.00 | 67.03 | C |
| ATOM | 2180 | CB | SER | A | 279 | 46.810 | −80.523 | −30.195 | 1.00 | 69.23 | C |
| ATOM | 2181 | OG | SER | A | 279 | 45.940 | −81.447 | −30.826 | 1.00 | 77.69 | O |
| ATOM | 2182 | C | SER | A | 279 | 44.803 | −79.215 | −29.433 | 1.00 | 64.40 | C |
| ATOM | 2183 | O | SER | A | 279 | 44.779 | −77.991 | −29.614 | 1.00 | 66.92 | O |
| ATOM | 2184 | N | TYR | A | 280 | 43.735 | −79.999 | −29.619 | 1.00 | 58.38 | N |
| ATOM | 2185 | CA | TYR | A | 280 | 42.381 | −79.439 | −29.753 | 1.00 | 56.80 | C |
| ATOM | 2186 | CB | TYR | A | 280 | 41.332 | −80.536 | −29.679 | 1.00 | 53.71 | C |
| ATOM | 2187 | CG | TYR | A | 280 | 41.373 | −81.476 | −30.839 | 1.00 | 53.81 | C |
| ATOM | 2188 | CD1 | TYR | A | 280 | 40.805 | −81.122 | −32.059 | 1.00 | 52.62 | C |
| ATOM | 2189 | CE1 | TYR | A | 280 | 40.838 | −81.988 | −33.137 | 1.00 | 55.93 | C |
| ATOM | 2190 | CZ | TYR | A | 280 | 41.450 | −83.230 | −33.001 | 1.00 | 58.31 | C |
| ATOM | 2191 | CH | TYR | A | 280 | 41.483 | −84.092 | −34.075 | 1.00 | 63.55 | O |
| ATOM | 2192 | CE2 | TYR | A | 280 | 42.016 | −83.609 | −31.794 | 1.00 | 56.10 | C |
| ATOM | 2193 | CD2 | TYR | A | 280 | 41.977 | −82.729 | −30.722 | 1.00 | 56.28 | C |
| ATOM | 2194 | C | TYR | A | 280 | 42.112 | −78.436 | −28.637 | 1.00 | 54.72 | C |
| ATOM | 2195 | O | TYR | A | 280 | 41.748 | −77.286 | −28.883 | 1.00 | 55.79 | O |
| ATOM | 2196 | N | LEU | A | 281 | 42.941 | −78.884 | −27.410 | 1.00 | 52.92 | N |
| ATOM | 2197 | CA | LEU | A | 281 | 42.120 | −78.081 | −26.236 | 1.00 | 54.20 | C |
| ATOM | 2198 | CB | LEU | A | 281 | 42.418 | −78.918 | −24.998 | 1.00 | 57.45 | C |
| ATOM | 2199 | CG | LEU | A | 281 | 41.318 | −79.135 | −23.959 | 1.00 | 58.99 | C |
| ATOM | 2200 | CD1 | LEU | A | 281 | 41.816 | −80.096 | −22.886 | 1.00 | 58.82 | C |
| ATOM | 2201 | CD2 | LEU | A | 281 | 40.916 | −77.807 | −23.339 | 1.00 | 64.07 | C |
| ATOM | 2202 | C | LEU | A | 281 | 42.985 | −76.824 | −26.276 | 1.00 | 57.18 | C |
| ATOM | 2203 | O | LEU | A | 281 | 42.482 | −75.727 | −26.051 | 1.00 | 57.70 | O |
| ATOM | 2204 | N | GLN | A | 282 | 44.272 | −76.991 | −26.593 | 1.00 | 60.13 | N |
| ATOM | 2205 | CA | GLN | A | 282 | 45.240 | −75.887 | −26.630 | 1.00 | 62.11 | C |
| ATOM | 2206 | CB | GLN | A | 282 | 46.611 | −76.382 | −27.102 | 1.00 | 31.84 | C |
| ATOM | 2207 | CG | GLN | A | 282 | 47.550 | −76.962 | −26.046 | 1.00 | 29.73 | C |
| ATOM | 2208 | CD | GLN | A | 282 | 48.857 | −77.465 | −26.665 | 1.00 | 87.66 | C |
| ATOM | 2209 | CE1 | GLN | A | 282 | 48.854 | −78.258 | −27.615 | 1.00 | 86.25 | O |
| ATOM | 2210 | NE2 | GLN | A | 282 | 49.981 | −76.988 | −26.137 | 1.00 | 87.71 | N |
| ATOM | 2211 | C | GLN | A | 282 | 44.779 | −74.801 | −27.585 | 1.00 | 59.68 | C |
| ATOM | 2212 | O | GLN | A | 282 | 45.022 | −73.617 | −27.366 | 1.00 | 54.94 | O |
| ATOM | 2213 | N | TYR | A | 283 | 44.119 | −75.216 | −28.661 | 1.00 | 61.05 | N |
| ATOM | 2214 | CA | TYR | A | 283 | 43.591 | −74.761 | −29.620 | 1.00 | 62.92 | C |
| ATOM | 2215 | CB | TYR | A | 283 | 43.108 | −74.947 | −30.901 | 1.00 | 62.02 | C |
| ATOM | 2216 | CG | TYR | A | 283 | 42.691 | −73.946 | −31.953 | 1.00 | 67.26 | C |
| ATOM | 2217 | CD1 | TYR | A | 283 | 43.632 | −73.105 | −32.558 | 1.00 | 66.91 | C |
| ATOM | 2218 | CE1 | TYR | A | 283 | 43.249 | −72.167 | −33.506 | 1.00 | 67.67 | C |
| ATOM | 2219 | CZ | TYR | A | 283 | 41.910 | −72.065 | −33.867 | 1.00 | 67.00 | C |
| ATOM | 2220 | OH | TYR | A | 283 | 41.514 | −71.142 | −34.815 | 1.00 | 63.62 | O |
| ATOM | 2221 | CE2 | TYR | A | 283 | 40.964 | −72.890 | −33.280 | 1.00 | 66.63 | C |
| ATOM | 2222 | CD2 | TYR | A | 283 | 41.354 | −73.819 | −32.328 | 1.00 | 66.08 | C |
| ATOM | 2223 | C | TYR | A | 283 | 42.484 | −73.403 | −28.996 | 1.00 | 65.06 | C |
| ATOM | 2224 | O | TYR | A | 283 | 42.493 | −72.174 | −29.146 | 1.00 | 65.17 | O |
| ATOM | 2225 | N | LEU | A | 284 | 41.556 | −74.051 | −28.286 | 1.00 | 59.97 | N |
| ATOM | 2226 | CA | LEU | A | 284 | 40.495 | −73.348 | −27.576 | 1.00 | 58.66 | C |
| ATOM | 2227 | CB | LEU | A | 284 | 39.589 | −74.325 | −26.836 | 1.00 | 59.17 | C |
| ATOM | 2228 | CG | LEU | A | 284 | 38.724 | −75.157 | −27.755 | 1.00 | 59.17 | C |
| ATOM | 2229 | CD1 | LEU | A | 284 | 37.759 | −75.995 | −26.934 | 1.00 | 61.59 | C |
| ATOM | 2230 | CD2 | LEU | A | 284 | 37.990 | −74.226 | −28.706 | 1.00 | 58.47 | C |
| ATOM | 2231 | C | LEU | A | 284 | 41.038 | −72.342 | −26.591 | 1.00 | 57.59 | C |
| ATOM | 2232 | O | LEU | A | 384 | 40.629 | −71.178 | −26.601 | 1.00 | 49.56 | O |
| ATOM | 2233 | N | GLU | A | 285 | 41.958 | −72.791 | −25.737 | 1.00 | 65.07 | N |
| ATOM | 2234 | CA | GLU | A | 285 | 42.594 | −71.892 | −24.768 | 1.00 | 72.96 | C |
| ATOM | 2235 | CB | GLU | A | 285 | 43.479 | −72.653 | −23.769 | 1.00 | 78.09 | C |
| ATOM | 2236 | CG | GLU | A | 285 | 42.666 | −73.149 | −22.569 | 1.00 | 96.06 | C |
| ATOM | 2237 | CD | GLU | A | 285 | 43.315 | −74.285 | −21.786 | 1.00 | 103.71 | C |
| ATOM | 2238 | OE1 | GLU | A | 285 | 44.183 | −74.996 | −22.339 | 1.00 | 108.88 | O |
| ATOM | 2239 | OE2 | GLU | A | 285 | 42.937 | −74.479 | −20.608 | 1.00 | 105.48 | O |
| ATOM | 2240 | C | GLU | A | 285 | 43.319 | −70.753 | −25.483 | 1.00 | 69.45 | C |
| ATOM | 2241 | O | GLU | A | 285 | 43.374 | −69.632 | −24.973 | 1.00 | 70.26 | O |
| ATOM | 2242 | N | TYR | A | 286 | 43.811 | −71.031 | −26.691 | 1.00 | 65.64 | N |
| ATOM | 2243 | CA | TYR | A | 286 | 44.426 | −70.004 | −27.521 | 1.00 | 63.16 | C |
| ATOM | 2244 | CB | TYR | A | 286 | 45.163 | −70.626 | −28.726 | 1.00 | 60.62 | C |
| ATOM | 2245 | CG | TYR | A | 286 | 45.402 | −69.689 | −29.889 | 1.00 | 61.69 | C |
| ATOM | 2246 | CD1 | TYR | A | 286 | 46.278 | −68.602 | −29.779 | 1.00 | 64.22 | C |
| ATOM | 2247 | CE1 | TYR | A | 286 | 46.485 | −67.734 | −30.848 | 1.00 | 65.79 | C |
| ATOM | 2248 | CZ | TYR | A | 286 | 45.823 | −67.960 | −32.056 | 1.00 | 70.27 | C |
| ATOM | 2249 | OH | TYR | A | 286 | 46.025 | −67.115 | −33.136 | 1.00 | 69.74 | O |
| ATOM | 2250 | CE2 | TYR | A | 286 | 44.959 | −69.039 | −32.187 | 1.00 | 66.61 | C |
| ATOM | 2251 | CD2 | TYR | A | 286 | 44.756 | −69.894 | −31.109 | 1.00 | 62.62 | C |
| ATOM | 2252 | C | TYR | A | 286 | 43.365 | −68.977 | −27.925 | 1.00 | 61.82 | C |
| ATOM | 2253 | O | TYR | A | 286 | 43.543 | −67.770 | −27.696 | 1.00 | 61.13 | O |
| ATOM | 2254 | N | LEU | A | 287 | 42.256 | −69.454 | −28.488 | 1.00 | 58.70 | N |
| ATOM | 2255 | CA | LEU | A | 287 | 41.137 | −68.568 | −28.857 | 1.00 | 60.85 | C |
| ATOM | 2256 | CB | LEU | A | 287 | 39.996 | −69.377 | −29.472 | 1.00 | 56.67 | C |
| ATOM | 2257 | CG | LEU | A | 287 | 39.805 | −69.304 | −30.989 | 1.00 | 56.37 | C |
| ATOM | 2258 | CD1 | LEU | A | 287 | 41.109 | −69.249 | −31.766 | 1.00 | 58.23 | C |
| ATOM | 2259 | CD2 | LEU | A | 287 | 38.968 | −70.480 | −31.448 | 1.00 | 53.86 | C |
| ATOM | 2260 | C | LEU | A | 287 | 40.620 | −67.626 | −27.739 | 1.00 | 60.14 | C |
| ATOM | 2261 | O | LEU | A | 287 | 40.410 | −66.426 | −27.987 | 1.00 | 57.06 | O |
| ATOM | 2262 | N | SER | A | 288 | 40.446 | −68.160 | −26.526 | 1.00 | 59.39 | N |
| ATOM | 2263 | CA | SER | A | 288 | 39.963 | −67.373 | −25.375 | 1.00 | 63.57 | C |
| ATOM | 2264 | CB | SER | A | 288 | 39.524 | −68.289 | −24.234 | 1.00 | 64.34 | C |
| ATOM | 2265 | OG | SER | A | 288 | 40.242 | −69.505 | −24.283 | 1.00 | 66.86 | O |
| ATOM | 2266 | C | SER | A | 288 | 40.965 | −66.357 | −24.850 | 1.00 | 67.70 | C |
| ATOM | 2267 | O | SER | A | 288 | 40.588 | −65.245 | −24.462 | 1.00 | 63.20 | O |
| ATOM | 2268 | N | GLN | A | 289 | 42.239 | −66.742 | −24.821 | 1.00 | 75.47 | N |
| ATOM | 2269 | CA | GLN | A | 289 | 43.295 | −65.805 | −24.460 | 1.00 | 81.78 | C |
| ATOM | 2270 | CB | GLN | A | 289 | 44.599 | −66.540 | −24.167 | 1.00 | 88.58 | C |
| ATOM | 2271 | CG | GLN | A | 289 | 44.660 | −67.055 | −22.732 | 1.00 | 99.92 | C |
| ATOM | 2272 | CD | GLN | A | 289 | 45.342 | −68.412 | −22.603 | 1.00 | 109.12 | C |
| ATOM | 2273 | OE1 | GLN | A | 289 | 46.309 | −68.710 | −23.313 | 1.00 | 107.93 | O |
| ATOM | 2274 | NE2 | GLN | A | 289 | 44.835 | −69.244 | −21.686 | 1.00 | 108.53 | N |
| ATOM | 2275 | C | GLN | A | 289 | 43.465 | −64.730 | −25.528 | 1.00 | 80.45 | C |
| ATOM | 2276 | O | GLN | A | 289 | 43.892 | −63.523 | −25.233 | 1.00 | 77.69 | O |
| ATOM | 2277 | N | ASN | A | 290 | 43.086 | −65.955 | −26.759 | 1.00 | 86.93 | N |
| ATOM | 2278 | CA | ASN | A | 290 | 43.137 | −64.111 | −27.878 | 1.00 | 91.52 | C |
| ATOM | 2279 | CB | ASN | A | 290 | 43.576 | −64.324 | −29.161 | 1.00 | 94.03 | C |
| ATOM | 2280 | CG | ASN | A | 290 | 45.080 | −64.362 | −29.326 | 1.00 | 99.74 | C |
| ATOM | 2281 | OD1 | ASN | A | 290 | 45.595 | −64.559 | −30.402 | 1.00 | 108.43 | O |
| ATOM | 2282 | ND2 | ASN | A | 290 | 45.795 | −65.233 | −28.263 | 1.00 | 95.42 | N |
| ATOM | 2283 | C | ASN | A | 290 | 41.833 | −63.360 | −28.140 | 1.00 | 92.01 | C |
| ATOM | 2284 | O | ASN | A | 290 | 41.581 | −62.920 | −29.264 | 1.00 | 95.17 | O |
| ATOM | 2285 | N | ARG | A | 291 | 40.993 | −63.221 | −27.123 | 1.00 | 89.73 | N |
| ATOM | 2286 | CA | ARG | A | 291 | 39.799 | −62.409 | −27.288 | 1.00 | 85.93 | C |
| ATOM | 2287 | CB | ARG | A | 291 | 38.551 | −63.127 | −26.755 | 1.00 | 80.92 | C |
| ATOM | 2288 | CG | ARG | A | 291 | 38.301 | −63.048 | −25.260 | 1.00 | 78.11 | C |
| ATOM | 2289 | CD | ARG | A | 291 | 36.955 | −63.589 | −24.947 | 1.00 | 83.18 | C |
| ATOM | 2290 | NE | ARG | A | 291 | 36.607 | −63.632 | −23.529 | 1.00 | 84.17 | N |
| ATOM | 2291 | CZ | ARG | A | 291 | 36.892 | −64.578 | −22.637 | 1.00 | 81.25 | C |
| ATOM | 2292 | NH1 | ARG | A | 291 | 37.540 | −65.681 | −22.988 | 1.00 | 81.24 | N |
| ATOM | 2293 | NH2 | ARG | A | 291 | 36.529 | −64.412 | −21.377 | 1.00 | 89.56 | N |
| ATOM | 2294 | C | ARG | A | 291 | 40.034 | −61.039 | −26.648 | 1.00 | 86.23 | C |
| ATOM | 2295 | G | ARG | A | 291 | 40.803 | −60.938 | −25.689 | 1.00 | 83.74 | O |
| ATOM | 2296 | N | PRO | A | 292 | 39.403 | −59.976 | −27.193 | 1.00 | 88.84 | N |
| ATOM | 2297 | CA | PRO | A | 292 | 39.617 | −58.628 | −26.650 | 1.00 | 88.12 | C |
| ATOM | 2298 | CB | PRO | A | 292 | 38.594 | −57.767 | −27.411 | 1.00 | 88.59 | C |
| ATOM | 2299 | CG | PRO | A | 292 | 37.630 | −58.734 | −28.024 | 1.00 | 91.86 | C |
| ATOM | 2300 | CD | PRO | A | 292 | 38.434 | −59.967 | −28.306 | 1.00 | 88.45 | C |
| ATOM | 2301 | C | PRO | A | 292 | 39.340 | −58.565 | −25.150 | 1.00 | 90.58 | C |
| ATOM | 2302 | O | PRO | A | 292 | 38.317 | −59.096 | −24.698 | 1.00 | 92.33 | O |
| ATOM | 2303 | N | PRO | A | 293 | 40.244 | −57.924 | −24.377 | 1.00 | 93.30 | N |
| ATOM | 2304 | CA | PRO | A | 293 | 40.024 | −57.823 | −22.928 | 1.00 | 95.38 | C |
| ATOM | 2305 | CB | PRO | A | 293 | 41.289 | −57.112 | −22.424 | 1.00 | 94.00 | C |
| ATOM | 2306 | CG | PRO | A | 293 | 41.785 | −56.340 | −23.601 | 1.00 | 92.70 | C |
| ATOM | 2307 | CD | PRO | A | 293 | 41.429 | −57.158 | −24.812 | 1.00 | 92.74 | C |
| ATOM | 2308 | C | PRO | A | 293 | 38.767 | −57.000 | −22.623 | 1.00 | 93.08 | C |
| ATOM | 2309 | C | PRO | A | 293 | 38.466 | −56.040 | −23.345 | 1.00 | 88.35 | O |
| ATOM | 2310 | N | PRO | A | 294 | 38.007 | −57.389 | −21.579 | 1.00 | 92.08 | N |
| ATOM | 2311 | CA | PRO | A | 294 | 36.763 | −56.689 | −21.260 | 1.00 | 90.93 | C |
| ATOM | 2312 | CB | PRO | A | 294 | 36.278 | −57.397 | −19.983 | 1.00 | 94.68 | C |
| ATOM | 2313 | CG | PRO | A | 294 | 37.483 | −58.075 | −19.414 | 1.00 | 88.66 | C |
| ATOM | 2314 | CD | PRO | A | 294 | 38.303 | −58.461 | −20.608 | 1.00 | 93.16 | C |
| ATOM | 2315 | C | PRO | A | 294 | 36.999 | −55.199 | −20.991 | 1.00 | 89.47 | C |
| ATOM | 2316 | O | PRO | A | 294 | 37.971 | −54.841 | −20.319 | 1.00 | 89.95 | O |
| ATOM | 2317 | N | ASN | A | 295 | 36.131 | −54.337 | −21.523 | 1.00 | 83.45 | N |
| ATOM | 2318 | CA | ASN | A | 295 | 36.222 | −52.900 | −21.243 | 1.00 | 75.80 | C |
| ATOM | 2319 | CB | ASN | A | 295 | 35.343 | −52.085 | −22.209 | 1.00 | 72.67 | C |
| ATOM | 2320 | CG | ASN | A | 295 | 33.848 | −52.243 | −21.944 | 1.00 | 70.29 | C |
| ATOM | 2321 | OD1 | ASN | A | 295 | 33.428 | −52.632 | −20.857 | 1.00 | 69.51 | O |
| ATOM | 2322 | ND2 | ASN | A | 295 | 33.039 | −51.919 | −22.943 | 1.00 | 65.33 | N |
| ATOM | 2323 | C | ASN | A | 295 | 35.900 | −52.614 | −19.770 | 1.00 | 72.63 | C |
| ATOM | 2324 | O | ASN | A | 295 | 35.627 | −53.543 | −19.005 | 1.00 | 78.41 | O |
| ATOM | 2325 | N | ALA | A | 296 | 35.928 | −51.349 | −19.365 | 1.00 | 67.15 | N |
| ATOM | 2326 | CA | ALA | A | 296 | 35.721 | −51.012 | −17.951 | 1.00 | 64.62 | C |
| ATOM | 2327 | CB | ALA | A | 296 | 36.087 | −49.557 | −17.692 | 1.00 | 60.13 | C |
| ATOM | 2328 | C | ALA | A | 296 | 34.291 | −51.331 | −17.452 | 1.00 | 66.65 | C |
| ATOM | 2329 | O | ALA | A | 296 | 34.116 | −51.813 | −16.321 | 1.00 | 63.73 | O |
| ATOM | 2330 | N | TYR | A | 297 | 33.289 | −51.067 | −18.302 | 1.00 | 63.63 | N |
| ATOM | 2331 | CA | TYR | A | 297 | 31.891 | −51.441 | −18.040 | 1.00 | 61.27 | C |
| ATOM | 2332 | CB | TYR | A | 297 | 30.990 | −50.993 | −19.202 | 1.00 | 59.85 | C |
| ATOM | 2333 | CG | TYR | A | 297 | 29.515 | −51.345 | −19.066 | 1.00 | 59.42 | C |
| ATOM | 2334 | CD1 | TYR | A | 297 | 29.043 | −52.628 | −19.360 | 1.00 | 59.12 | C |
| ATOM | 2335 | CE1 | TYR | A | 297 | 27.694 | −52.942 | −19.246 | 1.00 | 57.30 | C |
| ATOM | 2336 | CZ | TYR | A | 297 | 26.791 | −51.963 | −18.851 | 1.00 | 56.95 | C |
| ATOM | 2337 | OH | TYR | A | 297 | 25.455 | −52.264 | −18.745 | 1.00 | 56.63 | O |
| ATOM | 2338 | CE2 | TYR | A | 297 | 27.228 | −50.683 | −18.568 | 1.00 | 56.92 | C |
| ATOM | 2339 | CD2 | TYR | A | 297 | 26.581 | −50.382 | −18.679 | 1.00 | 60.93 | C |
| ATOM | 2340 | C | TYR | A | 297 | 31.747 | −52.945 | −17.792 | 1.00 | 59.72 | C |
| ATOM | 2341 | O | TYR | A | 297 | 31.100 | −53.351 | −16.832 | 1.00 | 60.13 | O |
| ATOM | 2342 | N | GLU | A | 298 | 32.372 | −53.753 | −18.650 | 1.00 | 63.24 | N |
| ATOM | 2343 | CA | GLU | A | 298 | 32.287 | −55.220 | −18.581 | 1.00 | 66.88 | C |
| ATOM | 2344 | CB | GLU | A | 298 | 32.837 | −55.868 | −19.862 | 1.00 | 68.80 | C |
| ATOM | 2345 | CG | GLU | A | 298 | 31.862 | −55.874 | −21.032 | 1.00 | 70.99 | C |
| ATOM | 2346 | CD | GLU | A | 298 | 32.536 | −55.951 | −22.401 | 1.00 | 77.73 | C |
| ATOM | 2347 | OE1 | GLU | A | 298 | 33.740 | −56.301 | −22.483 | 1.00 | 77.70 | O |
| ATOM | 2348 | OE2 | GLU | A | 298 | 31.848 | −55.654 | −23.408 | 1.00 | 72.34 | O |
| ATOM | 2349 | C | GLU | A | 298 | 32.969 | −55.808 | −17.348 | 1.00 | 68.02 | C |
| ATOM | 2350 | O | GLU | A | 298 | 32.598 | −56.886 | −16.889 | 1.00 | 71.88 | O |
| ATOM | 2351 | N | LEU | A | 299 | 33.960 | −55.101 | −16.815 | 1.00 | 67.86 | N |
| ATOM | 2352 | CA | LEU | A | 299 | 34.578 | −55.491 | −15.551 | 1.00 | 68.39 | C |
| ATOM | 2353 | CB | LEU | A | 299 | 33.971 | −54.840 | −15.398 | 1.00 | 74.98 | C |
| ATOM | 2354 | CG | LEU | A | 299 | 37.304 | −55.624 | −15.522 | 1.00 | 77.69 | C |
| ATOM | 2355 | CD1 | LEU | A | 299 | 37.157 | −57.079 | −15.964 | 1.00 | 74.87 | C |
| ATOM | 2356 | CD2 | LEU | A | 299 | 38.319 | −54.885 | −16.401 | 1.00 | 75.66 | C |
| ATOM | 2357 | C | LEU | A | 299 | 33.655 | −55.153 | −14.372 | 1.00 | 65.09 | C |
| ATOM | 2358 | O | LEU | A | 299 | 33.487 | −55.956 | −13.448 | 1.00 | 61.59 | O |
| ATOM | 2359 | N | PHE | A | 300 | 33.046 | −53.969 | −14.406 | 1.00 | 66.17 | N |
| ATOM | 2360 | CA | PHE | A | 300 | 32.086 | −53.593 | −13.359 | 1.00 | 65.25 | C |
| ATOM | 2361 | CB | PHE | A | 300 | 31.614 | −52.145 | −13.526 | 1.00 | 63.23 | C |
| ATOM | 2362 | CG | PHE | A | 300 | 30.481 | −51.772 | −12.605 | 1.00 | 66.59 | C |
| ATOM | 2363 | CD1 | PHE | A | 300 | 30.736 | −51.308 | −11.317 | 1.00 | 66.21 | C |
| ATOM | 2364 | CE1 | PHE | A | 300 | 29.692 | −50.983 | −10.460 | 1.00 | 64.65 | C |
| ATOM | 2365 | CZ | PHE | A | 300 | 28.378 | −51.120 | −10.885 | 1.00 | 65.09 | C |
| ATOM | 2366 | CE2 | PHE | A | 300 | 28.108 | −51.580 | −12.168 | 1.00 | 63.46 | C |
| ATOM | 2367 | CD2 | PHE | A | 300 | 29.151 | −51.906 | −13.018 | 1.00 | 65.38 | C |
| ATOM | 2368 | C | PHE | A | 300 | 30.875 | −54.532 | −13.327 | 1.00 | 60.61 | C |
| ATOM | 2369 | O | PHE | A | 300 | 30.474 | −55.000 | −12.275 | 1.00 | 59.86 | O |
| ATOM | 2370 | N | ALA | A | 301 | 30.310 | −54.793 | −14.500 | 1.00 | 61.60 | N |
| ATOM | 2371 | CA | ALA | A | 301 | 29.052 | −55.511 | −14.643 | 1.00 | 57.48 | C |
| ATOM | 2372 | CB | ALA | A | 301 | 28.440 | −55.177 | −15.990 | 1.00 | 53.67 | C |
| ATOM | 2373 | C | ALA | A | 301 | 29.231 | −57.511 | −14.508 | 1.00 | 60.57 | C |
| ATOM | 2374 | O | ALA | A | 301 | 28.267 | −57.768 | −14.596 | 1.00 | 61.20 | O |
| ATOM | 2375 | N | LYS | A | 302 | 30.464 | −57.431 | −14.254 | 1.00 | 69.00 | N |
| ATOM | 2376 | CA | LYS | A | 302 | 30.845 | −58.829 | −14.406 | 1.00 | 74.29 | C |
| ATOM | 2377 | CB | LYS | A | 302 | 32.340 | −59.043 | −14.139 | 1.00 | 79.58 | C |
| ATOM | 2378 | CG | LYS | A | 302 | 32.865 | −60.369 | −14.685 | 1.00 | 87.46 | C |
| ATOM | 2379 | CD | LYS | A | 302 | 32.592 | −60.523 | −16.181 | 1.00 | 87.83 | C |
| ATOM | 2380 | CE | LYS | A | 302 | 33.173 | −61.815 | −16.737 | 1.00 | 90.01 | C |
| ATOM | 2381 | NZ | LYS | A | 302 | 34.659 | −61.770 | −16.827 | 1.00 | 81.41 | N |
| ATOM | 2382 | C | LYS | A | 302 | 30.017 | −59.781 | −13.567 | 1.00 | 66.59 | C |
| ATOM | 2383 | O | LYS | A | 302 | 29.430 | −60.732 | −14.104 | 1.00 | 68.06 | O |
| ATOM | 2384 | N | GLY | A | 303 | 29.961 | −59.528 | −12.265 | 1.00 | 56.25 | N |
| ATOM | 2385 | CA | GLY | A | 303 | 29.218 | −60.418 | −11.374 | 1.00 | 55.43 | C |
| ATOM | 2386 | C | GLY | A | 303 | 27.692 | −60.374 | −11.511 | 1.00 | 54.01 | C |
| ATOM | 2387 | O | GLY | A | 303 | 27.006 | −61.161 | −10.860 | 1.00 | 51.71 | O |
| ATOM | 2388 | N | TYR | A | 304 | 27.172 | −59.485 | −12.376 | 1.00 | 51.07 | N |
| ATOM | 2389 | CA | TYR | A | 304 | 25.759 | −59.094 | −12.365 | 1.00 | 49.46 | C |
| ATOM | 2390 | CB | TYR | A | 304 | 25.652 | −57.663 | −12.294 | 1.00 | 51.71 | C |
| ATOM | 2391 | CG | TYR | A | 304 | 25.971 | −57.027 | −10.917 | 1.00 | 55.21 | C |
| ATOM | 2392 | CD1 | TYR | A | 304 | 25.038 | −57.129 | −9.891 | 1.00 | 55.05 | C |
| ATOM | 2393 | CE1 | TYR | A | 304 | 25.312 | −56.568 | −8.617 | 1.00 | 57.65 | C |
| ATOM | 2394 | CZ | TYR | A | 304 | 26.534 | −56.097 | −8.347 | 1.00 | 61.67 | C |
| ATOM | 2395 | OH | TYR | A | 304 | 26.761 | −55.653 | −7.064 | 1.00 | 65.94 | O |
| ATOM | 2396 | CE2 | TYR | A | 304 | 27.499 | −55.981 | −9.346 | 1.00 | 61.98 | C |
| ATOM | 2397 | CD2 | TYR | A | 304 | 27.215 | −56.449 | −10.627 | 1.00 | 57.44 | C |
| ATOM | 2398 | C | TYR | A | 304 | 24.906 | −59.630 | −13.509 | 1.00 | 48.18 | C |
| ATOM | 2399 | O | TYR | A | 304 | 23.709 | −59.342 | −13.585 | 1.00 | 48.72 | O |
| ATOM | 2400 | N | GLU | A | 305 | 25.522 | −60.406 | −14.392 | 1.00 | 46.96 | N |
| ATOM | 2401 | CA | GLU | A | 305 | 24.891 | −60.802 | −15.641 | 1.00 | 47.79 | C |
| ATOM | 2402 | CB | GLU | A | 305 | 25.877 | −61.369 | −16.522 | 1.00 | 50.96 | C |
| ATOM | 2403 | CG | GLU | A | 305 | 26.840 | −60.644 | −17.271 | 1.00 | 58.32 | C |
| ATOM | 2404 | CD | GLU | A | 305 | 28.232 | −61.238 | −17.424 | 1.00 | 68.14 | C |
| ATOM | 2405 | OE1 | GLU | A | 305 | 28.589 | −62.099 | −16.590 | 1.00 | 74.27 | O |
| ATOM | 2406 | OE2 | GLU | A | 305 | 28.975 | −60.845 | −18.360 | 1.00 | 67.11 | O |
| ATOM | 2407 | C | GLU | A | 305 | 23.619 | −61.384 | −15.368 | 1.00 | 48.63 | C |
| ATOM | 2408 | O | GLU | A | 305 | 23.619 | −62.341 | −14.590 | 1.00 | 45.47 | O |
| ATOM | 2409 | N | ASP | A | 306 | 22.518 | −61.226 | −15.960 | 1.00 | 47.34 | N |
| ATOM | 2410 | CA | ASP | A | 306 | 21.181 | −61.683 | −15.673 | 1.00 | 46.04 | C |
| ATOM | 2411 | CB | ASP | A | 306 | 20.951 | −62.998 | −16.437 | 1.00 | 42.38 | C |
| ATOM | 2412 | CG | ASP | A | 306 | 21.109 | −62.835 | −17.939 | 1.00 | 43.37 | C |
| ATOM | 2413 | OD1 | ASP | A | 306 | 20.857 | −61.734 | −18.466 | 1.00 | 43.80 | O |
| ATOM | 2414 | OD2 | ASP | A | 306 | 21.498 | −63.809 | −18.608 | 1.00 | 46.91 | O |
| ATOM | 2415 | C | ASP | A | 306 | 20.838 | −61.138 | −14.171 | 1.00 | 41.99 | C |
| ATOM | 2416 | O | ASP | A | 306 | 19.988 | −62.633 | −13.813 | 1.00 | 44.02 | O |
| ATOM | 2417 | N | TYR | A | 307 | 21.490 | −61.071 | −13.304 | 1.00 | 39.63 | N |
| ATOM | 2418 | CA | TYR | A | 307 | 21.060 | −60.974 | −11.916 | 1.00 | 43.34 | C |
| ATOM | 2419 | CB | TYR | A | 307 | 22.252 | −60.631 | −11.010 | 1.00 | 47.58 | C |
| ATOM | 2420 | CG | TYR | A | 307 | 21.918 | −60.596 | −9.540 | 1.00 | 55.83 | C |
| ATOM | 2421 | CD1 | TYR | A | 307 | 21.868 | −61.775 | −8.789 | 1.00 | 59.53 | C |
| ATOM | 2422 | CE1 | TYR | A | 307 | 21.548 | −61.748 | −7.436 | 1.00 | 44.55 | C |
| ATOM | 2423 | CZ | TYR | A | 307 | 21.274 | −60.528 | −6.816 | 1.00 | 68.45 | C |
| ATOM | 2424 | OH | TYR | A | 307 | 20.955 | −60.512 | −5.472 | 1.00 | 77.24 | O |
| ATOM | 2425 | CE2 | TYR | A | 307 | 21.316 | −59.342 | −7.535 | 1.00 | 62.51 | C |
| ATOM | 2426 | CD2 | TYR | A | 307 | 21.639 | −59.381 | −8.891 | 1.00 | 60.40 | C |
| ATOM | 2427 | C | TYR | A | 307 | 19.918 | −59.943 | −11.788 | 1.00 | 40.14 | C |
| ATOM | 2428 | O | TYR | A | 307 | 20.095 | −58.756 | −12.070 | 1.00 | 59.90 | O |
| ATOM | 2429 | N | LEU | A | 308 | 18.742 | −60.393 | −11.382 | 1.00 | 21.19 | N |
| ATOM | 2430 | CA | LEU | A | 308 | 17.559 | −59.510 | −11.342 | 1.00 | 36.99 | C |
| ATOM | 2431 | CB | LEU | A | 308 | 16.284 | −60.324 | −11.339 | 1.00 | 35.41 | C |
| ATOM | 2432 | CG | LEU | A | 308 | 15.851 | −61.180 | −12.519 | 1.00 | 54.76 | C |
| ATOM | 2433 | CD1 | LEU | A | 308 | 14.511 | −61.776 | −12.135 | 1.00 | 82.18 | C |
| ATOM | 2434 | CD2 | LEU | A | 308 | 15.725 | −60.376 | −13.797 | 1.00 | 82.92 | C |
| ATOM | 2435 | C | LEU | A | 308 | 17.536 | −58.683 | −10.084 | 1.00 | 99.33 | C |
| ATOM | 2436 | O | LEU | A | 308 | 17.617 | −59.233 | −8.980 | 1.00 | 38.66 | O |
| ATOM | 2437 | N | GLN | A | 309 | 17.416 | −57.366 | −10.230 | 1.00 | 40.12 | N |
| ATOM | 2438 | CA | GLN | A | 309 | 17.259 | −56.501 | −9.054 | 1.00 | 41.68 | C |
| ATOM | 2439 | CB | GLN | A | 309 | 18.337 | −55.426 | −9.005 | 1.00 | 45.66 | C |
| ATOM | 2440 | CG | GLN | A | 309 | 19.680 | −55.882 | −8.481 | 1.00 | 57.21 | C |
| ATOM | 2441 | CD | GLN | A | 309 | 20.786 | −55.006 | −9.032 | 1.00 | 66.75 | C |
| ATOM | 2442 | OE1 | GLN | A | 309 | 21.185 | −55.161 | −10.198 | 1.00 | 70.99 | O |
| ATOM | 2443 | NE2 | GLN | A | 309 | 21.268 | −54.053 | −8.215 | 1.00 | 60.30 | N |
| ATOM | 2444 | C | GLN | A | 309 | 15.906 | −55.815 | −9.023 | 1.00 | 39.69 | C |
| ATOM | 2445 | O | GLN | A | 309 | 15.356 | −55.409 | −10.054 | 1.00 | 37.64 | O |
| ATOM | 2446 | N | ASER | A | 310 | 15.479 | −55.489 | −7.811 | 0.50 | 42.05 | N |
| ATOM | 2447 | N | BSER | A | 310 | 15.304 | −55.855 | −7.845 | 0.50 | 37.77 | N |
| ATOM | 2448 | CA | ASER | A | 310 | 14.505 | −54.419 | −7.019 | 0.50 | 44.75 | C |
| ATOM | 2449 | CA | BSER | A | 310 | 13.868 | −55.662 | −7.695 | 0.50 | 35.27 | C |
| ATOM | 2450 | CB | ASER | A | 310 | 14.117 | −54.334 | −6.134 | 0.50 | 44.79 | C |
| ATOM | 2451 | C3 | BSER | A | 310 | 13.378 | −56.531 | −6.534 | 0.50 | 35.28 | C |
| ATOM | 2452 | O2 | ASER | A | 310 | 13.627 | −55.576 | −5.637 | 0.50 | 44.38 | O |
| ATOM | 2453 | O3 | BSER | A | 310 | 11.986 | −56.420 | −6.346 | 0.50 | 36.16 | O |
| ATOM | 2454 | C | ASER | A | 310 | 15.124 | −53.080 | −8.092 | 0.50 | 44.74 | C |
| ATOM | 2455 | C | BSER | A | 310 | 13.581 | −54.190 | −7.448 | 0.50 | 33.86 | C |
| ATOM | 2456 | O | ASER | A | 310 | 16.286 | −52.784 | −7.819 | 0.50 | 46.85 | O |
| ATOM | 2457 | O | BSER | A | 310 | 14.399 | −53.496 | −6.850 | 0.50 | 35.48 | O |
| ATOM | 2458 | N | APRO | A | 311 | 14.371 | −52.276 | −8.844 | 0.50 | 43.74 | N |
| ATOM | 2459 | N | BPRO | A | 311 | 12.427 | −53.709 | −7.914 | 0.50 | 33.31 | N |
| ATOM | 2460 | CA | APRO | A | 311 | 14.976 | −50.980 | −9.081 | 0.50 | 43.37 | C |
| ATOM | 2461 | CA | BPRO | A | 311 | 12.034 | −52.296 | −7.851 | 0.50 | 33.86 | C |
| ATOM | 2462 | CB | APRO | A | 311 | 13.948 | −50.285 | −9.977 | 0.50 | 40.91 | C |
| ATOM | 2463 | CB | BPRO | A | 311 | 10.586 | −52.304 | −8.356 | 0.50 | 35.57 | C |
| ATOM | 2464 | CG | APRO | A | 311 | 13.277 | −51.407 | −10.706 | 0.50 | 38.58 | C |
| ATOM | 2465 | CG | BPRO | A | 311 | 10.150 | −53.734 | −8.290 | 0.50 | 34.34 | C |
| ATOM | 2466 | CD | APRO | A | 311 | 13.164 | −52.485 | −9.664 | 0.50 | 42.19 | C |
| ATOM | 2467 | CD | BPRO | A | 311 | 11.384 | −54.539 | −8.537 | 0.50 | 34.14 | C |
| ATOM | 2468 | C | APRO | A | 311 | 15.133 | −50.226 | −7.758 | 0.50 | 46.08 | C |
| ATOM | 2469 | c | BPRO | A | 311 | 12.092 | −51.689 | −6.458 | 0.50 | 33.46 | C |
| ATOM | 2470 | O | APRO | A | 311 | 14.349 | −50.454 | −6.827 | 0.50 | 44.33 | O |
| ATOM | 2471 | O | BPRO | A | 311 | 11.739 | −52.329 | −5.467 | 0.50 | 32.00 | O |
| ATOM | 2472 | N | ALEU | A | 312 | 16.149 | −49.360 | −7.669 | 0.50 | 44.74 | N |
| ATOM | 2473 | N | BLEU | A | 312 | 12.540 | −50.447 | −6.390 | 0.50 | 35.65 | N |
| ATOM | 2474 | CA | ALEU | A | 312 | 16.310 | −48.485 | −6.511 | 0.50 | 42.33 | C |
| ATOM | 2475 | CA | BLEU | A | 312 | 12.772 | −49.803 | −5.100 | 0.50 | 35.79 | C |
| ATOM | 2476 | CB | ALEU | A | 312 | 17.629 | −47.714 | −6.584 | 0.50 | 40.90 | C |
| ATOM | 2477 | CB | BLEU | A | 312 | 14.264 | −49.652 | −4.855 | 0.50 | 36.45 | C |
| ATOM | 2478 | CG | ALEU | A | 312 | 18.952 | −48.388 | −6.201 | 0.50 | 38.16 | C |
| ATOM | 2479 | CG | BLEU | A | 312 | 14.920 | −48.982 | −6.054 | 0.50 | 35.83 | C |
| ATOM | 2480 | CD1 | ALEU | A | 312 | 20.058 | −47.374 | −6.401 | 0.50 | 36.05 | C |
| ATOM | 2481 | CD1 | BLEU | A | 312 | 16.354 | −49.487 | −6.176 | 0.50 | 33.01 | C |
| ATOM | 2482 | CD2 | ALEU | A | 312 | 18.963 | −48.309 | −4.762 | 0.50 | 36.61 | C |
| ATOM | 2483 | CD2 | BLEU | A | 312 | 14.101 | −49.343 | −7.286 | 0.50 | 35.75 | C |
| ATOM | 2484 | C | ALEU | A | 312 | 15.150 | −47.198 | −6.446 | 0.50 | 42.07 | C |
| ATOM | 2485 | C | BLEU | A | 312 | 12.135 | −48.433 | −5.013 | 0.50 | 32.95 | C |
| ATOM | 2486 | O | ALEU | A | 312 | 14.653 | −47.244 | −7.472 | 0.50 | 43.89 | O |
| ATOM | 2487 | O | BLEU | A | 312 | 11.746 | −47.340 | −6.023 | 0.50 | 29.86 | O |
| ATOM | 2488 | N | AGLN | A | 313 | 14.719 | −47.162 | −5.241 | 0.50 | 42.44 | N |
| ATOM | 2489 | N | BGLN | A | 313 | 12.078 | −47.833 | −3.785 | 0.50 | 32.01 | N |
| ATOM | 2490 | CA | AGLN | A | 313 | 13.609 | −46.229 | −5.069 | 0.50 | 45.90 | C |
| ATOM | 2491 | CA | BGLN | A | 313 | 11.437 | −46.571 | −3.472 | 0.50 | 31.07 | C |
| ATOM | 2492 | CB | AGLN | A | 313 | 12.316 | −47.911 | −4.776 | 0.50 | 47.26 | C |
| ATOM | 2493 | CB | BGLN | A | 313 | 10.663 | −46.346 | −2.169 | 0.50 | 31.30 | C |
| ATOM | 2494 | CG | AGLN | A | 313 | 11.027 | −46.721 | −4.927 | 0.50 | 49.65 | C |
| ATOM | 2495 | CG | BGLN | A | 313 | 9.718 | −48.039 | −2.206 | 0.50 | 29.24 | C |
| ATOM | 2496 | CD | AGLN | A | 313 | 9.770 | −47.880 | −4.764 | 0.50 | 51.29 | C |
| ATOM | 2497 | CD | BGLN | A | 313 | 8.706 | −47.905 | −3.316 | 0.50 | 29.02 | C |
| ATOM | 2498 | OE1 | AGLN | A | 313 | 9.098 | −47.938 | −3.724 | 0.50 | 53.19 | O |
| ATOM | 2499 | OE1 | BGLN | A | 313 | 8.307 | −46.792 | −3.658 | 0.50 | 31.11 | O |
| ATOM | 2500 | NE2 | AGLN | A | 313 | 9.453 | −47.369 | −5.789 | 0.50 | 46.81 | N |
| ATOM | 2501 | NE2 | BGLN | A | 313 | 8.311 | −49.023 | −3.913 | 0.50 | 27.47 | N |
| ATOM | 2502 | C | AGLN | A | 313 | 13.925 | −45.231 | −3.947 | 0.50 | 43.75 | C |
| ATOM | 2503 | C | BGLN | A | 313 | 12.428 | −45.508 | −3.368 | 0.50 | 32.20 | C |
| ATOM | 2504 | O | AGLN | A | 313 | 13.463 | −45.897 | −2.813 | 0.50 | 45.24 | O |
| ATOM | 2505 | O | BGLN | A | 313 | 13.002 | −45.285 | −2.299 | 0.50 | 32.52 | O |
| ATOM | 2506 | N | APRO | A | 314 | 14.723 | −44.196 | −4.261 | 0.50 | 41.78 | N |
| ATOM | 2507 | N | BPRO | A | 314 | 12.628 | −44.168 | −4.482 | 0.50 | 32.07 | N |
| ATOM | 2508 | CA | APRO | A | 314 | 15.202 | −43.262 | −3.246 | 0.50 | 42.67 | C |
| ATOM | 2509 | CA | BPRO | A | 314 | 13.604 | −43.376 | −4.651 | 0.50 | 34.29 | C |
| ATOM | 2510 | CB | APRO | A | 314 | 16.490 | −42.712 | −3.859 | 0.50 | 41.50 | C |
| ATOM | 2511 | CB | BPRO | A | 314 | 13.256 | −43.097 | −6.031 | 0.50 | 31.95 | C |
| ATOM | 2512 | CG | APRO | A | 314 | 16.295 | −42.133 | −5.341 | 0.50 | 42.04 | C |
| ATOM | 2513 | CG | BPRO | A | 314 | 11.910 | −43.630 | −6.365 | 0.50 | 32.15 | C |
| ATOM | 2514 | CD | APRO | A | 314 | 15.184 | −41.423 | −5.608 | 0.50 | 40.80 | C |
| ATOM | 2515 | CD | BPRO | A | 314 | 11.866 | −44.986 | −5.718 | 0.50 | 32.21 | C |
| ATOM | 2516 | C | APRO | A | 314 | 14.247 | −42.110 | −2.976 | 0.50 | 44.32 | C |
| ATOM | 2517 | C | BPRO | A | 314 | 13.422 | −42.577 | −3.630 | 0.50 | 36.55 | C |
| ATOM | 2518 | O | APRO | A | 314 | 14.706 | −41.012 | −2.708 | 0.50 | 45.20 | O |
| ATOM | 2519 | O | BPRO | A | 314 | 14.378 | −42.116 | −2.999 | 0.50 | 37.39 | O |
| ATOM | 2520 | N | ALEU | A | 315 | 12.939 | −42.350 | −3.047 | 0.50 | 45.62 | N |
| ATOM | 2521 | N | BLEU | A | 315 | 12.176 | −42.157 | −3.508 | 0.50 | 41.19 | N |
| ATOM | 2522 | CA | ALEU | A | 315 | 11.955 | −41.313 | −2.704 | 0.50 | 49.06 | C |
| ATOM | 2523 | CA | BLEU | A | 315 | 11.767 | −41.159 | −2.548 | 0.50 | 43.28 | C |
| ATOM | 2524 | CB | ALEU | A | 315 | 11.039 | −40.990 | −3.891 | 0.50 | 47.86 | C |
| ATOM | 2525 | CB | BLEU | A | 315 | 10.397 | −40.420 | −2.952 | 0.50 | 39.63 | C |
| ATOM | 2526 | CG | ALEU | A | 315 | 10.094 | −39.133 | −3.592 | 0.50 | 46.44 | C |
| ATOM | 2527 | CG | BLEU | A | 315 | 10.386 | −39.948 | −4.308 | 0.50 | 37.31 | C |
| ATOM | 2528 | CD1 | ALEU | A | 315 | 10.885 | −38.578 | −3.264 | 0.50 | 46.19 | C |
| ATOM | 2529 | CD1 | BLEU | A | 315 | 8.988 | −39.440 | −4.600 | 0.50 | 39.98 | C |
| ATOM | 2530 | CD2 | ALEU | A | 315 | 9.151 | −39.584 | −4.752 | 0.50 | 48.40 | C |
| ATOM | 2531 | CD2 | BLEU | A | 315 | 11.368 | −38.100 | −4.239 | 0.50 | 38.30 | C |
| ATOM | 2532 | C | ALEU | A | 315 | 11.114 | −41.621 | −1.452 | 0.50 | 49.79 | C |
| ATOM | 2533 | C | BLEU | A | 315 | 11.622 | −41.813 | −1.200 | 0.50 | 46.35 | C |
| ATOM | 2534 | O | ALEU | A | 315 | 10.689 | −40.104 | −0.742 | 0.50 | 51.40 | O |
| ATOM | 2535 | O | BLEU | A | 315 | 12.182 | −41.361 | −0.196 | 0.50 | 45.07 | O |
| ATOM | 2536 | N | MET | A | 316 | 10.872 | −42.905 | −1.201 | 1.00 | 48.33 | N |
| ATOM | 2537 | CA | MET | A | 316 | 10.248 | −43.376 | 0.018 | 1.00 | 51.87 | C |
| ATOM | 2538 | CB | MET | A | 316 | 9.007 | −44.166 | −0.379 | 1.00 | 57.01 | C |
| ATOM | 2539 | CG | MET | A | 316 | 8.335 | −45.012 | 0.670 | 1.00 | 63.32 | C |
| ATOM | 2540 | SD | MET | A | 316 | 6.877 | −45.756 | −0.098 | 1.00 | 79.84 | S |
| ATOM | 2541 | CE | MET | A | 316 | 5.713 | −44.391 | 0.043 | 1.00 | 67.94 | C |
| ATOM | 2542 | C | MET | A | 316 | 11.227 | −44.168 | 0.885 | 1.00 | 49.22 | C |
| ATOM | 2543 | O | MET | A | 316 | 10.995 | −44.340 | 2.074 | 1.00 | 52.35 | O |
| ATOM | 2544 | N | ASP | A | 317 | 12.320 | −44.642 | 0.290 | 1.00 | 47.47 | N |
| ATOM | 2545 | CA | ASP | A | 317 | 13.436 | −45.718 | 1.056 | 1.00 | 45.92 | C |
| ATOM | 2546 | CB | ASP | A | 317 | 13.972 | −46.507 | 0.428 | 1.00 | 49.51 | C |
| ATOM | 2547 | CG | ASP | A | 317 | 12.977 | −47.631 | 0.406 | 1.00 | 13.49 | C |
| ATOM | 2548 | CD1 | ASP | A | 317 | 12.161 | −47.776 | 1.339 | 1.00 | 55.89 | O |
| ATOM | 2549 | CD2 | ASP | A | 317 | 13.048 | −48.398 | −0.570 | 1.00 | 58.44 | O |
| ATOM | 2550 | C | ASP | A | 317 | 14.600 | −44.257 | 0.994 | 1.00 | 43.21 | C |
| ATOM | 2551 | O | ASP | A | 317 | 14.657 | −43.373 | 0.122 | 1.00 | 40.48 | O |
| ATOM | 2552 | N | ASN | A | 318 | 15.562 | −44.498 | 1.875 | 1.00 | 39.24 | N |
| ATOM | 2553 | CA | ASN | A | 318 | 16.831 | −43.788 | 1.874 | 1.00 | 37.61 | C |
| ATOM | 2554 | CB | ASN | A | 318 | 17.108 | −43.191 | 3.267 | 1.00 | 38.55 | C |
| ATOM | 2555 | CG | ASN | A | 318 | 16.262 | −41.968 | 3.566 | 1.00 | 40.41 | C |
| ATOM | 2556 | CD1 | ASN | A | 318 | 16.013 | −41.115 | 2.703 | 1.00 | 46.60 | O |
| ATOM | 2557 | ND2 | ASN | A | 318 | 15.826 | −41.864 | 4.794 | 1.00 | 44.53 | N |
| ATOM | 2558 | C | ASN | A | 318 | 17.985 | −44.695 | 1.439 | 1.00 | 84.86 | C |
| ATOM | 2559 | C | ASN | A | 318 | 18.226 | −45.768 | 1.996 | 1.00 | 84.16 | O |
| ATOM | 2560 | N | LEU | A | 319 | 18.711 | −44.243 | 0.442 | 1.00 | 84.00 | N |
| ATOM | 2561 | CA | LEU | A | 319 | 19.850 | −44.979 | −0.065 | 1.00 | 83.80 | C |
| ATOM | 2562 | CB | LEU | A | 319 | 20.404 | −44.235 | −1.291 | 1.00 | 12.24 | C |
| ATOM | 2563 | CG | LEU | A | 319 | 19.676 | −44.429 | −2.611 | 1.00 | 30.15 | C |
| ATOM | 2564 | CD1 | LEU | A | 319 | 20.331 | −43.606 | −3.694 | 1.00 | 29.22 | C |
| ATOM | 2565 | CD2 | LEU | A | 319 | 19.751 | −45.888 | −2.992 | 1.00 | 19.64 | C |
| ATOM | 2566 | C | LEU | A | 319 | 20.941 | −45.165 | 1.020 | 1.00 | 34.01 | C |
| ATOM | 2567 | C | LEU | A | 319 | 21.185 | −44.275 | 1.838 | 1.00 | 12.14 | O |
| ATOM | 2568 | N | GLU | A | 320 | 21.560 | −46.332 | 1.033 | 1.00 | 35.48 | N |
| ATOM | 2569 | CA | GLU | A | 320 | 22.676 | −46.616 | 1.927 | 1.00 | 42.04 | C |
| ATOM | 2570 | CB | GLU | A | 320 | 22.899 | −48.112 | 2.018 | 1.00 | 45.93 | C |
| ATOM | 2571 | CG | GLU | A | 320 | 21.748 | −48.917 | 2.592 | 1.00 | 54.62 | C |
| ATOM | 2572 | CD | GLU | A | 320 | 22.053 | −50.399 | 2.520 | 1.00 | 64.08 | C |
| ATOM | 2573 | CE1 | GLU | A | 320 | 22.315 | −50.904 | 1.399 | 1.00 | 62.97 | O |
| ATOM | 2574 | CE2 | GLU | A | 320 | 22.000 | −51.054 | 3.589 | 1.00 | 72.24 | O |
| AIOM | 2575 | C | GLU | A | 320 | 24.001 | −45.975 | 1.487 | 1.00 | 42.67 | C |
| ATOM | 2576 | O | GLU | A | 320 | 24.264 | −45.778 | 0.297 | 1.00 | 44.02 | O |
| ATOM | 2577 | N | SER | A | 321 | 24.844 | −45.691 | 2.470 | 1.00 | 44.26 | N |
| ATOM | 2578 | CA | SER | A | 321 | 26.147 | −45.083 | 2.244 | 1.00 | 45.78 | C |
| ATOM | 2579 | CB | SER | A | 321 | 26.948 | −45.096 | 3.543 | 1.00 | 44.90 | C |
| ATOM | 2580 | CG | SER | A | 321 | 28.262 | −44.665 | 3.327 | 1.00 | 44.99 | O |
| ATOM | 2581 | C | SER | A | 321 | 26.924 | −45.789 | 1.132 | 1.00 | 46.87 | C |
| ATOM | 2582 | O | 5ER | A | 321 | 27.462 | −45.122 | 0.235 | 1.00 | 48.24 | O |
| ATOM | 2583 | N | GLN | A | 322 | 26.954 | −47.125 | 1.172 | 1.00 | 44.84 | N |
| ATOM | 2584 | CA | GLN | A | 322 | 27.722 | −47.902 | 0.190 | 1.00 | 45.84 | C |
| AT0M | 2585 | CB | GLN | A | 322 | 27.969 | −49.332 | 0.660 | 1.00 | 49.57 | C |
| ATOM | 2586 | CG | GLN | A | 322 | 29.204 | −49.426 | 1.540 | 1.00 | 55.35 | C |
| ATOM | 2587 | CD | GLN | A | 322 | 29.502 | −50.837 | 2.003 | 1.00 | 63.51 | C |
| ATOM | 2588 | OE1 | GLN | A | 322 | 29.761 | −51.734 | 1.200 | 1.00 | 63.16 | O |
| ATOM | 2589 | NE2 | GLN | A | 322 | 29.472 | −51.037 | 3.313 | 1.00 | 69.17 | N |
| ATOM | 2590 | C | GLN | A | 322 | 27.126 | −47.884 | −1.202 | 1.00 | 45.39 | C |
| ATOM | 2591 | O | GLN | A | 322 | 27.778 | −48.300 | −2.166 | 1.00 | 67.76 | O |
| ATOM | 2592 | N | THR | A | 323 | 25.904 | −47.376 | −1.310 | 1.00 | 40.42 | N |
| ATOM | 2593 | CA | THR | A | 323 | 25.234 | −47.316 | −2.578 | 1.00 | 38.84 | C |
| ATOM | 2594 | CB | THR | A | 323 | 23.717 | −47.476 | −2.396 | 1.00 | 37.19 | C |
| ATOM | 2595 | OG1 | THR | A | 323 | 23.459 | −48.832 | 2.013 | 1.00 | 38.57 | O |
| ATOM | 2596 | CG2 | THR | A | 323 | 22.960 | −47.170 | −3.688 | 1.00 | 33.93 | C |
| ATOM | 2597 | C | THR | A | 323 | 25.610 | −46.022 | −3.278 | 1.00 | 40.45 | C |
| ATOM | 2598 | O | THR | A | 323 | 25.784 | −45.986 | −4.492 | 1.00 | 42.56 | O |
| ATOM | 2599 | N | TYR | A | 324 | 25.730 | −44.957 | −2.503 | 1.00 | 40.77 | N |
| ATOM | 2600 | CA | TYR | A | 324 | 26.274 | −43.727 | −3.011 | 1.00 | 59.85 | C |
| ATOM | 2601 | CB | TYR | A | 324 | 26.209 | −42.643 | −1.956 | 1.00 | 40.41 | C |
| ATOM | 2602 | CG | TYR | A | 324 | 24.834 | −42.084 | −1.663 | 1.00 | 38.76 | C |
| ATOM | 2603 | CD1 | TYR | A | 324 | 24.154 | −41.302 | −2.614 | 1.00 | 39.09 | C |
| ATOM | 2604 | CE1 | TYR | A | 324 | 22.891 | −40.769 | −2.345 | 1.00 | 35.79 | C |
| ATOM | 2605 | C2 | TYR | A | 324 | 22.303 | −41.003 | −1.100 | 1.00 | 38.80 | C |
| ATOM | 2606 | OH | TYR | A | 324 | 21.040 | −40.480 | −0.823 | 1.00 | 44.40 | O |
| ATOM | 2607 | CE2 | TYR | A | 324 | 22.960 | −41.763 | −0.142 | 1.00 | 35.93 | C |
| ATOM | 2608 | CD2 | TYR | A | 324 | 24.218 | −42.299 | −0.419 | 1.00 | 35.69 | C |
| ATOM | 2609 | C | TYR | A | 324 | 27.730 | −43.982 | −3.378 | 1.00 | 42.60 | C |
| ATOM | 2610 | O | TYR | A | 324 | 28.190 | −43.541 | −4.430 | 1.00 | 44.36 | O |
| ATOM | 2611 | N | GLU | A | 325 | 28.444 | −44.714 | −2.524 | 1.00 | 44.20 | N |
| ATOM | 2612 | CA | GLU | A | 325 | 29.848 | −45.005 | −2.775 | 1.00 | 47.39 | C |
| ATOM | 2613 | CB | GLU | A | 325 | 30.417 | −45.904 | −1.703 | 1.00 | 47.67 | C |
| ATOM | 2614 | CG | GLU | A | 325 | 31.894 | −46.157 | −1.883 | 1.00 | 48.73 | C |
| ATOM | 2615 | CD | GLU | A | 325 | 32.393 | −47.297 | −1.034 | 1.00 | 54.80 | C |
| ATOM | 2616 | OE1 | GLU | A | 325 | 31.619 | −48.242 | −0.754 | 1.00 | 56.84 | O |
| ATOM | 2617 | OE2 | GLU | A | 325 | 33.575 | −47.246 | −0.649 | 1.00 | 56.61 | O |
| ATOM | 2618 | C | GLU | A | 325 | 30.100 | −45.637 | −4.148 | 1.00 | 52.74 | C |
| ATOM | 2619 | O | GLU | A | 325 | 30.907 | −45.114 | −4.925 | 1.00 | 53.35 | O |
| ATOM | 2620 | N | VAL | A | 326 | 29.417 | −46.743 | −4.458 | 1.00 | 54.00 | N |
| ATOM | 2621 | CA | VAL | A | 326 | 29.688 | −47.447 | −5.724 | 1.00 | 50.97 | C |
| ATOM | 2622 | CB | VAL | A | 326 | 28.894 | −48.774 | −5.907 | 1.00 | 54.35 | C |
| ATOM | 2623 | CG1 | VAL | A | 326 | 29.347 | −49.827 | −4.907 | 1.00 | 53.29 | C |
| ATOM | 2624 | CG2 | VAL | A | 326 | 27.387 | −48.553 | −5.808 | 1.00 | 54.68 | C |
| ATOM | 2625 | C | VAL | A | 326 | 29.466 | −46.521 | −6.919 | 1.00 | 49.99 | C |
| ATOM | 2626 | O | VAL | A | 326 | 30.285 | −46.503 | −7.837 | 1.00 | 46.73 | O |
| ATOM | 2627 | N | PHE | A | 327 | 28.392 | −45.728 | −6.875 | 1.00 | 46.38 | N |
| ATOM | 2628 | CA | PHE | A | 327 | 27.998 | −44.901 | −8.019 | 1.00 | 45.43 | C |
| ATOM | 2629 | CB | PHE | A | 327 | 26.500 | −44.555 | −8.000 | 1.00 | 46.68 | C |
| ATOM | 2630 | CG | PHE | A | 327 | 25.710 | −45.388 | −8.795 | 1.00 | 49.26 | C |
| ATOM | 2631 | CD1 | PHE | A | 327 | 25.849 | −46.533 | −9.519 | 1.00 | 47.35 | C |
| ATOM | 2632 | CE1 | PHE | A | 327 | 25.140 | −47.684 | −9.789 | 1.00 | 48.85 | C |
| ATOM | 2633 | CZ | PHE | A | 327 | 24.280 | −48.207 | −8.829 | 1.00 | 48.35 | C |
| ATOM | 2634 | CE2 | PHE | A | 327 | 24.152 | −47.587 | −7.600 | 1.00 | 45.24 | C |
| ATOM | 2635 | CD2 | PHE | A | 327 | 24.861 | −46.433 | −7.341 | 1.00 | 47.54 | C |
| ATOM | 2636 | C | PHE | A | 327 | 28.762 | −43.605 | −8.194 | 1.00 | 45.41 | C |
| AIOM | 2637 | O | PHE | A | 327 | 28.837 | −43.070 | −9.299 | 1.00 | 48.11 | O |
| ATOM | 2618 | N | GLU | A | 328 | 29.329 | −43.096 | −7.111 | 1.00 | 46.70 | N |
| ATOM | 2619 | CA | GLU | A | 328 | 30.219 | −41.931 | −7.174 | 1.00 | 44.98 | C |
| ATOM | 2640 | CB | GLU | A | 328 | 30.561 | −41.446 | −5.756 | 1.00 | 41.37 | C |
| ATOM | 2641 | CG | GLU | A | 328 | 29.428 | −40.691 | −5.073 | 1.00 | 42.54 | C |
| ATOM | 2642 | CD | GLU | A | 328 | 29.518 | −40.693 | −3.547 | 1.00 | 44.28 | C |
| ATOM | 2643 | OE1 | GLU | A | 328 | 30.521 | −41.207 | −2.996 | 1.00 | 39.63 | O |
| ATOM | 2644 | OE2 | GLU | A | 328 | 28.579 | −40.170 | −2.893 | 1.00 | 38.94 | O |
| ATOM | 2645 | C | GLU | A | 328 | 31.495 | −42.270 | −7.962 | 1.00 | 45.92 | C |
| ATOM | 2646 | O | GLU | A | 328 | 32.238 | −41.384 | −8.369 | 1.00 | 43.10 | O |
| ATOM | 2647 | N | LYS | A | 329 | 31.708 | −43.562 | −8.200 | 1.00 | 47.24 | N |
| ATOM | 2648 | CA | LYS | A | 329 | 32.926 | −44.059 | −8.826 | 1.00 | 52.52 | C |
| ATOM | 2649 | CB | LYS | A | 329 | 33.216 | −45.482 | −8.324 | 1.00 | 56.02 | C |
| ATOM | 265O | CG | LYS | A | 329 | 34.684 | −45.301 | −8.064 | 1.00 | 66.22 | C |
| ATOM | 2651 | CD | LYS | A | 329 | 34.975 | −47.811 | −8.157 | 1.00 | 79.40 | C |
| ATOM | 2652 | CE | LYS | A | 329 | 35.156 | −47.320 | −9.597 | 1.00 | 73.39 | C |
| ATOM | 2653 | NZ | LYS | A | 329 | 34.846 | −49.370 | −9.774 | 1.00 | 62.93 | N |
| ATOM | 2654 | C | LYS | A | 329 | 32.840 | −44.009 | −10.370 | 1.00 | 52.87 | C |
| ATOM | 2655 | O | LYS | A | 329 | 33.788 | −44.892 | −11.076 | 1.00 | 54.12 | O |
| ATOM | 2656 | N | ASP | A | 330 | 31.719 | −43.526 | −10.892 | 1.00 | 45.74 | N |
| ATOM | 2657 | CA | ASP | A | 330 | 31.524 | −43.462 | −12.337 | 1.00 | 49.42 | C |
| ATOM | 2658 | CB | ASP | A | 330 | 30.042 | −43.376 | −12.653 | 1.00 | 46.46 | C |
| ATOM | 2659 | CG | ASP | A | 330 | 29.710 | −43.546 | −14.101 | 1.00 | 49.08 | C |
| ATOM | 2660 | OD1 | ASP | A | 330 | 30.364 | −42.949 | −14.984 | 1.00 | 47.34 | O |
| ATOM | 2661 | OD2 | ASP | A | 330 | 28.763 | −44.828 | −14.355 | 1.00 | 46.51 | O |
| ATOM | 2662 | C | ASP | A | 330 | 32.368 | −42.840 | −12.969 | 1.00 | 52.07 | C |
| ATOM | 2663 | O | ASP | A | 330 | 32.286 | −41.188 | −12.555 | 1.00 | 52.16 | O |
| ATOM | 2664 | N | PRO | A | 331 | 33.199 | −42.374 | −13.971 | 1.00 | 57.13 | N |
| ATOM | 2665 | CA | PRO | A | 331 | 33.969 | −41.550 | −14.506 | 1.00 | 63.51 | C |
| ATOM | 2666 | CB | PRO | A | 331 | 34.931 | −42.211 | −15.458 | 1.00 | 63.52 | C |
| ATOM | 2667 | CG | PRO | A | 331 | 34.803 | −43.701 | −15.306 | 1.00 | 63.05 | C |
| ATOM | 2668 | CD | PRO | A | 331 | 33.459 | −41.936 | −14.589 | 1.00 | 58.51 | C |
| ATOM | 2669 | C | PRO | A | 331 | 33.048 | −40.592 | −15.262 | 1.00 | 51.78 | C |
| ATOM | 2670 | O | PRO | A | 331 | 32.898 | −39.414 | −14.883 | 1.00 | 57.85 | O |
| ATOM | 2671 | N | ILE | A | 332 | 32.373 | −41.142 | −16.261 | 1.00 | 53.53 | N |
| ATOM | 2672 | CA | ILE | A | 332 | 31.688 | −40.364 | −17.275 | 1.00 | 48.92 | C |
| ATOM | 2673 | CB | ILE | A | 332 | 31.362 | −41.243 | −18.506 | 1.00 | 48.77 | C |
| ATOM | 2674 | CG1 | ILE | A | 332 | 32.478 | −42.290 | −18.736 | 1.00 | 47.89 | C |
| ATOM | 7675 | CD1 | ILE | A | 332 | 33.872 | −41.762 | −19.123 | 1.00 | 52.14 | C |
| ATOM | 2676 | CG2 | ILE | A | 332 | 31.209 | −40.378 | −19.751 | 1.00 | 53.43 | C |
| ATOM | 2677 | C | ILE | A | 332 | 30.476 | −39.557 | −16.772 | 1.00 | 47.35 | C |
| ATOM | 2678 | O | ILE | A | 332 | 30.266 | −38.425 | −17.232 | 1.00 | 44.32 | O |
| AIOM | 2679 | N | LYS | A | 333 | 29.708 | −40.093 | −15.814 | 1.00 | 42.58 | N |
| ATOM | 2680 | CA | LYS | A | 333 | 28.486 | −39.387 | −15.401 | 1.00 | 43.57 | C |
| ATOM | 2681 | CB | LYS | A | 333 | 27.646 | −40.178 | −14.386 | 1.00 | 43.79 | C |
| ATOM | 2682 | CG | LYS | A | 333 | 26.178 | −35.750 | −14.374 | 1.00 | 41.90 | C |
| ATOM | 2683 | CD | LYS | A | 333 | 25.402 | −40.375 | −13.225 | 1.00 | 43.60 | C |
| ATOM | 2684 | CE | LYS | A | 333 | 25.782 | −35.785 | −11.882 | 1.00 | 47.23 | C |
| ATOM | 2685 | NZ | LYS | A | 333 | 24.718 | −35.917 | −10.844 | 1.00 | 41.88 | N |
| ATOM | 2686 | C | LYS | A | 333 | 28.747 | −37.965 | −14.899 | 1.00 | 42.51 | C |
| ATOM | 2687 | O | LYS | A | 333 | 28.038 | −37.011 | −15.337 | 1.00 | 44.47 | O |
| ATOM | 2688 | N | TYR | A | 334 | 29.706 | −37.819 | −13.997 | 1.00 | 41.94 | N |
| ATOM | 2689 | CA | TYR | A | 334 | 29.934 | −36.523 | −13.385 | 1.00 | 45.11 | C |
| ATOM | 2690 | CB | TYR | A | 334 | 30.600 | −38.693 | −12.029 | 1.00 | 46.53 | C |
| ATOM | 2691 | CG | TYR | A | 334 | 29.774 | −37.558 | −11.112 | 1.00 | 45.40 | C |
| ATOM | 2692 | CD1 | TYR | A | 334 | 28.545 | −37.094 | −10.600 | 1.00 | 47.73 | C |
| ATOM | 2693 | CE1 | TYR | A | 334 | 27.754 | −37.892 | −9.789 | 1.00 | 43.14 | C |
| ATOM | 2694 | CZ | TYR | A | 334 | 28.188 | −35.170 | −9.475 | 1.00 | 45.37 | C |
| ATOM | 2695 | CH | TYR | A | 334 | 27.421 | −39.960 | −8.656 | 1.00 | 41.01 | O |
| ATOM | 2696 | CE2 | TYR | A | 334 | 29.403 | −39.654 | −9.970 | 1.00 | 44.10 | C |
| ATOM | 2697 | CD2 | TYR | A | 334 | 30.175 | −38.857 | −10.802 | 1.00 | 41.61 | C |
| ATOM | 2698 | C | TYR | A | 334 | 30.694 | −35.559 | −14.300 | 1.00 | 49.55 | C |
| ATOM | 2699 | O | TYR | A | 334 | 30.359 | −34.364 | −14.350 | 1.00 | 47.33 | O |
| ATOM | 2700 | N | SER | A | 335 | 31.077 | −30.086 | −15.042 | 1.00 | 50.49 | N |
| ATOM | 2701 | CA | SER | A | 335 | 32.416 | −35.315 | −16.059 | 1.00 | 52.01 | C |
| ATOM | 2702 | CB | SER | A | 335 | 33.409 | −36.190 | −16.806 | 1.00 | 54.14 | C |
| ATOM | 2703 | CG | SER | A | 335 | 34.564 | −36.368 | −16.024 | 1.00 | 70.83 | O |
| ATOM | 2704 | C | SER | A | 335 | 31.491 | −34.710 | −17.072 | 1.00 | 48.15 | C |
| ATOM | 2705 | O | SER | A | 335 | 31.671 | −33.569 | −17.485 | 1.00 | 49.72 | O |
| ATOM | 2706 | N | GLN | A | 336 | 30.522 | −35.508 | −17.494 | 1.00 | 46.33 | N |
| ATOM | 2707 | CA | GLN | A | 336 | 29.431 | −35.034 | −18.328 | 1.00 | 45.96 | C |
| ATOM | 2708 | CB | GLN | A | 336 | 28.564 | −36.714 | −18.789 | 1.00 | 49.90 | C |
| ATOM | 2709 | CG | GLN | A | 336 | 29.235 | −37.140 | −19.813 | 1.00 | 47.78 | C |
| ATOM | 2710 | CD | GLN | A | 336 | 29.396 | −36.488 | −21.175 | 1.00 | 46.58 | C |
| ATOM | 2711 | 0E1 | GLN | A | 336 | 30.509 | −36.227 | −21.606 | 1.00 | 51.35 | O |
| ATOM | 2712 | NE2 | GLN | A | 336 | 28.287 | −36.201 | −21.842 | 1.00 | 45.67 | N |
| ATOM | 2713 | C | GLN | A | 336 | 28.565 | −33.945 | −17.683 | 1.00 | 41.98 | C |
| ATOM | 2714 | O | GLN | A | 336 | 28.127 | −33.034 | −18.375 | 1.00 | 41.84 | O |
| ATOM | 2715 | N | TYR | A | 337 | 28.291 | −34.038 | −16.386 | 1.00 | 41.45 | N |
| ATOM | 2716 | CA | TYR | A | 337 | 22.485 | −32.993 | −15.714 | 1.00 | 42.09 | C |
| ATOM | 2717 | CB | TYR | A | 337 | 26.966 | −33.446 | −14.332 | 1.00 | 38.91 | C |
| AIOM | 2718 | CG | TYR | A | 337 | 25.559 | −34.069 | −14.786 | 1.00 | 41.93 | C |
| ATOM | 2719 | CD1 | TYR | A | 337 | 24.435 | −33.283 | −14.047 | 1.00 | 41.24 | C |
| ATOM | 2720 | CE1 | TYR | A | 337 | 23.169 | −33.838 | −13.986 | 1.00 | 41.40 | C |
| ATOM | 2721 | CZ | TYR | A | 337 | 23.008 | −35.210 | −14.146 | 1.00 | 42.05 | C |
| ATOM | 2722 | OH | TYR | A | 337 | 21.734 | −35.763 | −14.077 | 1.00 | 40.90 | O |
| ATOM | 2723 | CE2 | TYR | A | 337 | 24.111 | −36.018 | −14.362 | 1.00 | 38.55 | C |
| ATOM | 2724 | CD2 | TYR | A | 337 | 25.368 | −35.449 | −14.433 | 1.00 | 40.20 | C |
| ATOM | 2725 | C | TYR | A | 337 | 28.320 | −31.696 | −15.579 | 1.00 | 43.07 | C |
| ATOM | 2726 | O | TYR | A | 337 | 27.807 | −30.581 | −15.757 | 1.00 | 40.25 | O |
| ATOM | 2727 | N | GLN | A | 338 | 29.604 | −31.860 | −15.273 | 1.00 | 41.47 | N |
| ATOM | 2728 | CA | GLN | A | 338 | 30.523 | −30.737 | −15.197 | 1.00 | 44.04 | C |
| ATOM | 2729 | CB | GLN | A | 338 | 31.951 | −31.225 | −14.905 | 1.00 | 44.81 | C |
| ATOM | 2730 | CG | GLN | A | 338 | 32.887 | −30.120 | −14.433 | 1.00 | 45.01 | C |
| ATOM | 2731 | CD | GLN | A | 338 | 34.301 | −30.624 | −14.218 | 1.00 | 48.93 | C |
| ATOM | 2732 | OE1 | GLN | A | 338 | 34.521 | −31.625 | −13.530 | 1.00 | 49.47 | O |
| ATOM | 2733 | NE2 | GLN | A | 338 | 35.271 | −29.932 | −14.809 | 1.00 | 46.24 | N |
| ATOM | 2734 | C | GLN | A | 338 | 30.460 | −29.962 | −16.513 | 1.00 | 42.90 | C |
| ATOM | 2735 | O | GLN | A | 338 | 30.117 | −28.770 | −16.521 | 1.00 | 41.11 | O |
| ATOM | 2736 | N | GLN | A | 339 | 30.711 | −30.664 | −17.622 | 1.00 | 41.43 | N |
| ATOM | 2737 | CA | GLN | A | 339 | 30.640 | −30.063 | −18.942 | 1.00 | 41.76 | C |
| ATOM | 2738 | CB | GLN | A | 339 | 31.052 | −31.062 | −20.018 | 1.00 | 43.91 | C |
| ATOM | 2739 | CG | GLN | A | 339 | 31.270 | −30.454 | −21.411 | 1.00 | 49.59 | C |
| ATOM | 2740 | CD | GLN | A | 339 | 32.544 | −29.598 | −21.527 | 1.00 | 52.90 | C |
| ATOM | 2741 | OE1 | GLN | A | 339 | 33.475 | −29.702 | −20.713 | 1.00 | 52.89 | O |
| ATOM | 2742 | NE2 | GLN | A | 339 | 32.589 | −28.756 | −22.556 | 1.00 | 50.52 | N |
| ATOM | 2743 | C | GLN | A | 339 | 29.270 | −29.428 | −19.240 | 1.00 | 44.48 | C |
| ATOM | 2744 | O | GLN | A | 339 | 29.185 | −28.432 | −19.941 | 1.00 | 46.88 | O |
| ATOM | 2745 | N | ALA | A | 340 | 28.196 | −29.980 | −18.698 | 1.00 | 45.29 | N |
| ATOM | 2746 | CA | ALA | A | 340 | 26.880 | −29.393 | −18.954 | 1.00 | 47.01 | C |
| ATOM | 2747 | CB | ALA | A | 340 | 25.775 | −30.360 | −18.565 | 1.00 | 46.98 | C |
| ATOM | 2748 | C | ALA | A | 340 | 26.714 | −28.080 | −18.207 | 1.00 | 48.31 | C |
| ATOM | 2749 | O | ALA | A | 340 | 26.131 | −27.123 | −18.733 | 1.00 | 46.93 | O |
| ATOM | 2750 | N | ILE | A | 341 | 27.215 | −28.053 | −16.969 | 1.00 | 47.63 | N |
| ATOM | 2751 | CA | ILE | A | 341 | 27.120 | −26.869 | −16.133 | 1.00 | 48.37 | C |
| ATOM | 2752 | CB | ILE | A | 341 | 27.426 | −27.189 | −14.652 | 1.00 | 45.47 | C |
| ATOM | 2753 | CG1 | ILE | A | 341 | 26.485 | −28.290 | −14.146 | 1.00 | 44.41 | C |
| ATOM | 2754 | CD1 | ILE | A | 341 | 27.011 | −29.057 | −12.951 | 1.00 | 39.22 | C |
| ATOM | 2755 | CG2 | ILE | A | 341 | 27.284 | −25.939 | −13.779 | 1.00 | 41.17 | C |
| ATOM | 2756 | C | ILE | A | 341 | 28.061 | −25.798 | −16.699 | 1.00 | 50.17 | C |
| ATOM | 2757 | O | ILE | A | 341 | 27.690 | −24.632 | −16.800 | 1.00 | 46.59 | O |
| ATOM | 2758 | N | TYR | A | 342 | 29.258 | −26.231 | −17.089 | 1.00 | 55.36 | N |
| ATOM | 2759 | CA | TYR | A | 342 | 30.244 | −25.393 | −17.771 | 1.00 | 60.36 | C |
| ATOM | 2760 | CB | TYR | A | 342 | 31.425 | −26.249 | −18.247 | 1.00 | 62.78 | C |
| ATOM | 2761 | CG | TYR | A | 342 | 32.432 | −25.490 | −19.065 | 1.00 | 70.59 | C |
| ATOM | 2762 | CD1 | TYR | A | 342 | 33.507 | −24.838 | −18.457 | 1.00 | 74.11 | O |
| ATOM | 2763 | CE1 | TYR | A | 342 | 34.435 | −24.135 | −19.209 | 1.00 | 76.55 | C |
| ATOM | 2764 | CZ | TYR | A | 342 | 34.287 | −24.074 | −20.589 | 1.00 | 78.71 | C |
| ATOM | 2765 | OH | TYR | A | 342 | 35.191 | −23.384 | −21.356 | 1.00 | 75.51 | O |
| ATOM | 2766 | CE2 | TYR | A | 342 | 33.227 | −24.712 | −21.211 | 1.00 | 78.53 | C |
| ATOM | 2767 | CD2 | TYR | A | 342 | 32.313 | −25.418 | −20.450 | 1.00 | 73.57 | C |
| ATOM | 2768 | C | TYR | A | 342 | 29.644 | −24.582 | −18.930 | 1.00 | 60.24 | C |
| ATOM | 2769 | O | TYR | A | 342 | 29.641 | −23.153 | −18.868 | 1.00 | 60.39 | O |
| ATOM | 2770 | N | LYS | A | 343 | 29.138 | −25.266 | −19.962 | 1.00 | 55.89 | N |
| ATOM | 2771 | CA | LYS | A | 343 | 78.510 | −24.509 | −21.117 | 1.00 | 56.02 | C |
| ATOM | 2772 | CB | LYS | A | 343 | 27.979 | −25.516 | −22.146 | 1.00 | 54.77 | C |
| ATOM | 2773 | CG | LYS | A | 343 | 29.027 | −26.266 | −23.044 | 1.00 | 57.30 | C |
| ATOM | 2774 | CD | LYS | A | 343 | 28.378 | −26.371 | −24.285 | 1.00 | 58.55 | C |
| ATOM | 2775 | CE | LYS | A | 343 | 29.415 | −27.156 | −25.282 | 1.00 | 60.03 | C |
| ATOM | 2776 | NZ | LYS | A | 343 | 28.817 | −27.181 | −26.631 | 1.00 | 61.80 | N |
| ATOM | 2777 | C | LYS | A | 343 | 27.368 | −23.391 | −20.705 | 1.00 | 56.45 | C |
| ATOM | 2778 | O | LYS | A | 343 | 27.285 | −22.552 | −21.172 | 1.00 | 62.04 | O |
| ATOM | 2779 | N | CYS | A | 344 | 26.494 | −24.171 | −19.828 | 1.00 | 51.66 | N |
| ATOM | 2780 | CA | CYS | A | 344 | 25.313 | −23.188 | −19.455 | 1.00 | 50.44 | C |
| ATOM | 2781 | CB | CYS | A | 344 | 24.388 | −24.180 | −18.535 | 1.00 | 47.82 | C |
| ATOM | 2782 | SG | CYS | A | 344 | 22.938 | −23.333 | −18.010 | 1.00 | 50.30 | S |
| ATOM | 2783 | C | CYS | A | 344 | 25.674 | −22.050 | −18.810 | 1.00 | 49.96 | C |
| ATOM | 2784 | O | CYS | A | 344 | 25.011 | −21.039 | −19.044 | 1.00 | 47.82 | O |
| ATOM | 2785 | N | LEU | A | 345 | 26.717 | −22.041 | −17.990 | 1.00 | 50.83 | N |
| ATOM | 2786 | CA | LEU | A | 345 | 27.143 | −20.108 | −17.352 | 1.00 | 54.27 | C |
| ATOM | 2787 | CB | LEU | A | 345 | 28.287 | −21.471 | −16.367 | 1.00 | 52.24 | C |
| ATOM | 2788 | CG | LEU | A | 345 | 27.927 | −21.119 | −15.082 | 1.00 | 49.46 | C |
| ATOM | 2789 | CD1 | LEU | A | 345 | 79.174 | −22.359 | −14.320 | 1.00 | 48.33 | C |
| ATOM | 2790 | CD2 | LEU | A | 345 | 26.959 | −21.045 | −14.196 | 1.00 | 45.62 | C |
| ATOM | 2791 | C | LEU | A | 345 | 27.568 | −19.797 | −18.420 | 1.00 | 59.52 | C |
| ATOM | 2792 | O | LEU | A | 345 | 27.103 | −18.655 | −18.428 | 1.00 | 63.52 | O |
| ATOM | 2793 | N | LEU | A | 346 | 28.417 | −20.344 | −19.343 | 1.00 | 58.16 | N |
| ATOM | 2794 | CA | LEU | A | 346 | 28.935 | −19.378 | −20.395 | 1.00 | 45.94 | C |
| ATOM | 2795 | CB | LEU | A | 346 | 29.940 | −20.121 | −21.286 | 1.00 | 47.50 | C |
| ATOM | 2796 | CG | LEU | A | 346 | 31.197 | −20.496 | −20.476 | 1.00 | 48.37 | C |
| ATOM | 2797 | CD1 | LEU | A | 346 | 32.357 | −21.036 | −21.306 | 1.00 | 50.59 | C |
| ATOM | 2798 | CD2 | LEU | A | 346 | 31.672 | −19.327 | −19.633 | 1.00 | 45.61 | C |
| ATOM | 2799 | C | LEU | A | 346 | 27.813 | −18.724 | −21.188 | 1.00 | 40.81 | C |
| ATOM | 2800 | O | LEU | A | 346 | 27.904 | −17.549 | −21.548 | 1.00 | 71.44 | O |
| ATOM | 2801 | N | ASP | A | 347 | 26.732 | −19.467 | −21.396 | 1.00 | 61.84 | N |
| ATOM | 2802 | CA | ASP | A | 347 | 25.618 | −18.996 | −22.211 | 1.00 | 63.45 | C |
| ATOM | 2803 | CB | ASP | A | 347 | 24.806 | −20.183 | −22.754 | 1.00 | 65.12 | C |
| ATOM | 2804 | CG | ASP | A | 347 | 25.641 | −21.131 | −23.612 | 1.00 | 69.34 | C |
| ATOM | 2805 | CD1 | ASP | A | 347 | 26.893 | −21.046 | −23.602 | 1.00 | 66.50 | O |
| ATOM | 2806 | CD2 | ASP | A | 347 | 25.034 | −21.973 | −24.301 | 1.00 | 71.68 | O |
| ATOM | 2807 | C | ASP | A | 347 | 24.708 | −18.029 | −21.468 | 1.00 | 60.07 | C |
| ATOM | 2808 | O | ASP | A | 347 | 23.821 | −17.419 | −22.066 | 1.00 | 62.46 | O |
| ATOM | 2809 | N | ARG | A | 348 | 24.919 | −17.892 | −20.166 | 1.00 | 58.81 | N |
| ATOM | 2810 | CA | ARG | A | 348 | 24.005 | −17.099 | −19.343 | 1.00 | 59.73 | C |
| ATOM | 2811 | CB | ARG | A | 348 | 23.341 | −17.970 | −18.267 | 1.00 | 58.16 | C |
| ATOM | 2812 | CG | ARG | A | 348 | 22.218 | −18.850 | −18.812 | 1.00 | 53.68 | C |
| ATOM | 2813 | CD | ARG | A | 348 | 21.633 | −19.748 | −17.737 | 1.00 | 58.68 | C |
| ATOM | 2814 | NE | ARG | A | 348 | 21.051 | −18.987 | −16.641 | 1.00 | 55.16 | N |
| ATOM | 2815 | CZ | ARG | A | 348 | 19.878 | −18.464 | −16.659 | 1.00 | 54.85 | C |
| ATOM | 2816 | NH1 | ARG | A | 348 | 19.033 | −18.625 | −17.711 | 1.00 | 51.43 | N |
| ATOM | 2817 | NH2 | ARG | A | 348 | 19.396 | −17.788 | −15.612 | 1.00 | 50.91 | N |
| ATOM | 2818 | C | ARG | A | 348 | 24.657 | −15.86B | −18.725 | 1.00 | 59.56 | C |
| ATOM | 2819 | O | ARG | A | 348 | 23.951 | −14.930 | −18.311 | 1.00 | 55.26 | O |
| ATOM | 2820 | N | VAL | A | 349 | 25.993 | −15.888 | −18.664 | 1.00 | 59.01 | N |
| ATOM | 2821 | CA | VAL | A | 349 | 26.791 | −14.757 | −18.186 | 1.00 | 60.97 | C |
| ATOM | 2822 | CB | VAL | A | 349 | 27.215 | −14.882 | −16.701 | 1.00 | 62.45 | C |
| ATOM | 2823 | CG1 | VAL | A | 349 | 25.995 | −14.883 | −15.793 | 1.00 | 64.54 | C |
| ATOM | 2824 | CG2 | VAL | A | 349 | 28.038 | −16.126 | −16.465 | 1.00 | 60.52 | C |
| ATOM | 2825 | C | VAL | A | 349 | 27.996 | −14.461 | −19.098 | 1.00 | 64.49 | C |
| ATOM | 2826 | O | VAL | A | 349 | 28.957 | −15.221 | −19.128 | 1.00 | 50.00 | O |
| ATOM | 2827 | N | PRO | A | 350 | 27.900 | −13.333 | −19.839 | 1.00 | 67.85 | N |
| ATOM | 2828 | CA | PRO | A | 350 | 28.869 | −12.885 | −20.832 | 1.00 | 68.02 | C |
| ATOM | 2829 | CB | PRO | A | 350 | 28.176 | −11.6S5 | −21.491 | 1.00 | 68.46 | C |
| ATOM | 2830 | CG | PRO | A | 350 | 26.772 | −11.671 | −20.980 | 1.00 | 70.69 | C |
| ATOM | 2831 | CD | PRO | A | 350 | 26.834 | −12.337 | −19.646 | 1.00 | 68.39 | C |
| ATOM | 2832 | C | PRO | A | 350 | 30.137 | −12.421 | −20.157 | 1.00 | 66.94 | C |
| VTOM | 2833 | O | PRO | A | 350 | 30.080 | −11.942 | −19.024 | 1.00 | 66.29 | O |
| ATOM | 2834 | N | GLU | A | 351 | 31.258 | −12.563 | −20.861 | 1.00 | 69.62 | N |
| ATOM | 2835 | CA | GLU | A | 351 | 32.592 | −12.173 | −20.372 | 1.00 | 77.11 | C |
| ATOM | 2836 | CB | GLU | A | 351 | 33.595 | −12.148 | −21.531 | 1.00 | 81.39 | C |
| ATOM | 2837 | CZ | GLU | A | 351 | 35.048 | −12.030 | −21.102 | 1.00 | 86.98 | C |
| ATOM | 2838 | CD | GLU | A | 351 | 35.559 | −13.273 | −20.402 | 1.00 | 95.11 | C |
| ATOM | 2839 | OE1 | GLU | A | 351 | 35.104 | −13.566 | −19.274 | 1.00 | 97.90 | O |
| ATOM | 2840 | 0E2 | GLU | A | 351 | 36.432 | −13.952 | −20.978 | 1.00 | 98.19 | O |
| ATOM | 2841 | C | GLU | A | 351 | 32.655 | −10.850 | −19.600 | 1.00 | 74.61 | C |
| ATOM | 2842 | O | GLU | A | 351 | 33.243 | −10.786 | −18.525 | 1.00 | 71.61 | O |
| ATOM | 2843 | N | GLU | A | 352 | 32.039 | −9.8O8 | −20.148 | 1.00 | 76.90 | N |
| ATOM | 2844 | CA | GLU | A | 352 | 31.993 | −8.503 | −19.494 | 1.00 | 77.94 | C |
| ATOM | 2845 | CB | GLU | A | 352 | 31.441 | −7.434 | −20.455 | 1.00 | 82.61 | C |
| ATOM | 2846 | CG | GLU | A | 352 | 29.956 | −7.5S5 | −20.809 | 1.00 | 88.82 | C |
| ATOM | 2847 | CD | GLU | A | 352 | 29.663 | −8.421 | −22.039 | 1.00 | 91.45 | C |
| ATOM | 2848 | OE1 | GLU | A | 352 | 30.592 | −9.043 | −22.616 | 1.00 | 84.79 | O |
| ATOM | 2849 | 0E2 | GLU | A | 352 | 28.476 | −8.475 | −22.438 | 1.00 | 91.14 | O |
| ATOM | 2850 | C | GLU | A | 352 | 31.223 | −8.503 | −18.157 | 1.00 | 77.34 | C |
| ATOM | 2851 | O | GLU | A | 352 | 31.262 | −7.518 | −17.412 | 1.00 | 77.65 | O |
| ATOM | 2852 | N | GLU | A | 353 | 30.537 | −9.607 | −17.850 | 1.00 | 73.50 | N |
| ATOM | 2853 | CA | GLU | A | 353 | 29.761 | −9.708 | −16.605 | 1.00 | 67.47 | C |
| ATOM | 2854 | CB | GLU | A | 353 | 28.306 | −10.066 | −16.893 | 1.00 | 65.38 | C |
| ATOM | 2855 | CG | GLU | A | 353 | 27.630 | −9.100 | −17.846 | 1.00 | 66.23 | C |
| ATOM | 2856 | CD | GLU | A | 353 | 26.146 | −8.940 | −17.580 | 1.00 | 68.37 | C |
| ATOM | 2857 | OE1 | GLU | A | 353 | 25.743 | −8.850 | −16.396 | 1.00 | 64.82 | O |
| ATOM | 2858 | OE2 | GLU | A | 353 | 25.382 | −8.887 | −18.566 | 1.00 | 72.34 | O |
| ATOM | 2859 | C | GLU | A | 353 | 30.368 | −10.679 | −15.597 | 1.00 | 66.60 | C |
| ATOM | 2860 | O | GLU | A | 353 | 79.886 | −10.306 | −14.471 | 1.00 | 61.56 | O |
| ATOM | 2861 | N | LYS | A | 354 | 31.461 | −11.321 | −15.996 | 1.00 | 66.95 | N |
| ATOM | 2862 | CA | LYS | A | 354 | 32.023 | −12.432 | −13.256 | 1.00 | 67.64 | C |
| ATOM | 2863 | CB | LYS | A | 354 | 33.325 | −12.907 | −15.903 | 1.00 | 67.18 | C |
| ATOM | 2864 | CG | LYS | A | 354 | 34.523 | −11.993 | −15.717 | 1.00 | 70.74 | C |
| ATOM | 2865 | CD | LYS | A | 354 | 35.774 | −12.697 | −16.197 | 1.00 | 71.61 | C |
| ATOM | 2866 | CE | LYS | A | 354 | 36.992 | −12.212 | −15.435 | 1.00 | 76.32 | C |
| ATOM | 7867 | NZ | LYS | A | 354 | 38.055 | −13.251 | −15.398 | 1.00 | 71.37 | N |
| ATOM | 2868 | C | LYS | A | 354 | 32.188 | −12.208 | −13.748 | 1.00 | 78.13 | C |
| ATOM | 2869 | O | LYS | A | 354 | 31.940 | −13.122 | −12.951 | 1.00 | 88.73 | O |
| ATOM | 2870 | N | ASP | A | 355 | 32.584 | −10.997 | −13.362 | 1.00 | 80.91 | N |
| ATOM | 2871 | CA | ASP | A | 355 | 32.837 | −10.671 | −11.957 | 1.00 | 79.07 | C |
| ATOM | 2872 | CB | ASP | A | 355 | 34.018 | −9.705 | −11.837 | 1.00 | 82.85 | C |
| ATOM | 2873 | CG | ASP | A | 355 | 35.351 | −10.375 | −12.107 | 1.00 | 88.25 | C |
| ATOM | 2874 | OD1 | ASP | A | 355 | 36.148 | −9.816 | −12.890 | 1.00 | 88.57 | O |
| ATOM | 2875 | OD2 | ASP | A | 355 | 35.604 | −11.460 | −11.542 | 1.00 | 90.80 | O |
| ATOM | 2876 | C | ASP | A | 355 | 31.617 | −10.376 | −11.266 | 1.00 | 77.97 | C |
| ATOM | 2877 | O | ASP | A | 355 | 31.548 | −10.351 | −10.036 | 1.00 | 76.64 | O |
| ATOM | 2878 | N | THR | A | 356 | 30.658 | −9.500 | −12.057 | 1.00 | 77.86 | N |
| ATOM | 2879 | CA | THR | A | 356 | 29.479 | −8.116 | −11.518 | 1.00 | 80.32 | C |
| ATOM | 2880 | CB | THR | A | 356 | 29.144 | −7.542 | −12.334 | 1.00 | 77.53 | C |
| ATOM | 2881 | OG1 | THR | A | 356 | 28.134 | −6.396 | −11.650 | 1.00 | 84.35 | O |
| ATOM | 2882 | CG2 | THR | A | 356 | 28.643 | −7.976 | −13.737 | 1.00 | 78.64 | C |
| ATOM | 2883 | C | THR | A | 356 | 28.242 | −9.827 | −11.386 | 1.00 | 81.10 | C |
| ATOM | 2884 | O | THR | A | 356 | 27.335 | −9.562 | −10.587 | 1.00 | 74.92 | O |
| ATOM | 2885 | N | ASN | A | 357 | 28.215 | −10.598 | −12.174 | 1.00 | 79.48 | N |
| ATOM | 2886 | CA | ASN | A | 357 | 27.079 | −11.307 | −12.182 | 1.00 | 75.53 | C |
| ATOM | 2887 | CB | ASN | A | 357 | 26.551 | −12.3319 | −13.601 | 1.00 | 75.50 | C |
| ATOM | 2888 | CG | ASN | A | 357 | 25.169 | −12.522 | −13.616 | 1.00 | 74.77 | r |
| ATOM | 2889 | OD1 | ASN | A | 357 | 24.899 | −13.592 | −12.916 | 1.00 | 77.39 | O |
| ATOM | 2890 | ND2 | ASN | A | 357 | 24.285 | −12.055 | −14.416 | 1.00 | 72.58 | N |
| ATOM | 2891 | C | ASN | A | 357 | 27.395 | −13.138 | −11.509 | 1.00 | 72.97 | C |
| ATOM | 2892 | O | ASN | A | 357 | 28.207 | −13.943 | −11.997 | 1.00 | 73.74 | O |
| ATOM | 2893 | N | VAL | A | 558 | 26.746 | −13.135 | −10.368 | 1.00 | 64.87 | N |
| ATOM | 2894 | CA | VAL | A | 358 | 26.860 | −14.553 | −9.597 | 1.00 | 66.61 | C |
| ATOM | 2895 | CB | VAL | A | 358 | 27.111 | −14.337 | −8.093 | 1.00 | 69.88 | C |
| ATOM | 2896 | CG1 | VAL | A | 358 | 26.006 | −13.155 | −7.510 | 1.00 | 67.68 | C |
| ATOM | 2897 | CG2 | VAL | A | 358 | 27.305 | −15.112 | −7.280 | 1.00 | 69.62 | C |
| ATOM | 2898 | C | VAL | A | 358 | 25.604 | −15.402 | −9.860 | 1.00 | 61.23 | C |
| ATOM | 2899 | O | VAL | A | 358 | 24.476 | −14.999 | −9.530 | 1.00 | 52.25 | O |
| ATOM | 290O | N | GLN | A | 359 | 25.790 | −16.145 | −10.517 | 1.00 | 59.34 | N |
| ATOM | 2901 | CA | GLN | A | 359 | 24.634 | −17.406 | −10.847 | 1.00 | 64.48 | C |
| ATOM | 2902 | CB | GLN | A | 359 | 24.955 | −18.357 | −12.015 | 1.00 | 63.75 | C |
| ATOM | 2903 | CG | GLN | A | 359 | 24.916 | −17.733 | −13.412 | 1.00 | 65.06 | C |
| ATOM | 2904 | CD | GLN | A | 359 | 23.542 | −17.199 | −13.827 | 1.00 | 62.54 | C |
| ATOM | 2905 | OE1 | GLN | A | 359 | 23.185 | −16.465 | −13.521 | 1.00 | 60.33 | O |
| ATOM | 2906 | NE2 | GLN | A | 359 | 22.786 | −18.1106 | −14.558 | 1.00 | 61.09 | N |
| ATOM | 2907 | C | GLN | A | 359 | 24.188 | −18.211 | −9.624 | 1.00 | 58.47 | C |
| ATOM | 2908 | O | GLN | A | 359 | 25.036 | −18.760 | −8.911 | 1.00 | 55.73 | O |
| ATOM | 2909 | N | VAL | A | 360 | 22.880 | −18.261 | −9.361 | 1.00 | 54.20 | N |
| ATOM | 2910 | CA | VAL | A | 360 | 22.369 | −19.754 | −8.306 | 1.00 | 53.98 | C |
| ATOM | 2911 | CB | VAL | A | 360 | 21.187 | −18.159 | −7.481 | 1.00 | 63.85 | C |
| ATOM | 2912 | CG1 | VAL | A | 360 | 20.184 | −17.121 | −8.359 | 1.00 | 69.52 | C |
| ATOM | 2913 | CG2 | VAL | A | 360 | 20.482 | −19.630 | −6.652 | 1.00 | 61.43 | C |
| ATOM | 2914 | C | VAL | A | 360 | 22.073 | −20.559 | −8.848 | 1.00 | 51.34 | C |
| ATOM | 2915 | O | VAL | A | 360 | 21.154 | −20.760 | −9.664 | 1.00 | 43.96 | O |
| ATOM | 2916 | N | LEU | A | 361 | 22.893 | −21.506 | −8.376 | 1.00 | 46.62 | N |
| ATOM | 2917 | CA | LEU | A | 361 | 22.817 | −22.824 | −8.698 | 1.00 | 45.84 | C |
| ATOM | 2918 | CB | LEU | A | 361 | 24.203 | −23.416 | −9.058 | 1.00 | 52.50 | C |
| ATOM | 2919 | CG | LEU | A | 361 | 24.378 | −23.626 | −10.548 | 1.00 | 56.07 | C |
| ATOM | 2920 | CD1 | LEU | A | 361 | 25.827 | −23.871 | −10.902 | 1.00 | 49.31 | C |
| ATOM | 2921 | C02 | LEU | A | 361 | 23.530 | −24.832 | −10.913 | 1.00 | 60.65 | C |
| ATOM | 2922 | C | LEU | A | 361 | 22.243 | −23.831 | −7.587 | 1.00 | 46.73 | C |
| ATOM | 2923 | O | LEU | A | 361 | 22.715 | −23.815 | −6.449 | 1.00 | 44.44 | O |
| ATOM | 2924 | N | MET | A | 362 | 21.229 | −24.629 | −7.925 | 1.00 | 42.99 | N |
| ATOM | 2925 | CA | MET | A | 362 | 20.649 | −25.556 | −6.965 | 1.00 | 41.48 | C |
| ATOM | 2926 | CB | MET | A | 362 | 19.193 | −25.210 | −6.740 | 1.00 | 42.26 | C |
| ATOM | 2927 | CG | MET | A | 362 | 19.039 | −23.879 | −6.033 | 1.00 | 47.89 | C |
| ATOM | 2928 | SD | MET | A | 362 | 17.317 | −23.435 | −5.893 | 1.00 | 50.39 | S |
| ATOM | 2929 | CE | MET | A | 362 | 17.429 | −21.837 | −5.095 | 1.00 | 52.97 | C |
| ATOM | 2930 | C | MET | A | 362 | 20.808 | −27.017 | −7.374 | 1.00 | 42.00 | C |
| ATOM | 2931 | O | MET | A | 362 | 20.360 | −27.434 | −8.437 | 1.00 | 18.58 | O |
| ATOM | 2932 | N | VAL | A | 363 | 21.491 | −27.791 | −6.539 | 1.00 | 43.19 | N |
| ATOM | 2933 | CA | VAL | A | 363 | 21.554 | −29.232 | −6.751 | 1.00 | 41.41 | C |
| ATOM | 2934 | CB | VAL | A | 363 | 22.871 | −29.804 | −6.272 | 1.00 | 39.51 | C |
| ATOM | 2935 | CG1 | VAL | A | 363 | 22.915 | −31.291 | −6.561 | 1.00 | 39.63 | C |
| ATOM | 2936 | CG2 | VAL | A | 363 | 24.018 | −29.080 | −6.966 | 1.00 | 40.01 | C |
| ATOM | 2937 | C | VAL | A | 363 | 20.399 | −29.901 | −6.011 | 1.00 | 39.93 | C |
| ATOM | 2938 | C | VAL | A | 363 | 20.271 | −29.770 | −4.793 | 1.00 | 39.20 | O |
| ATOM | 2939 | N | LEU | A | 364 | 19.568 | −30.626 | −6.744 | 1.00 | 40.01 | N |
| ATOM | 2940 | CA | LEU | A | 364 | 18.311 | −31.139 | −6.166 | 1.00 | 39.68 | C |
| ATOM | 2941 | CB | LEU | A | 364 | 17.109 | −30.727 | −7.017 | 1.00 | 35.33 | C |
| ATOM | 2942 | CG | LEU | A | 364 | 16.898 | −29.209 | −7.097 | 1.00 | 35.81 | C |
| ATOM | 2943 | CD1 | LEU | A | 364 | 15.513 | −28.831 | −7.639 | 1.00 | 34.47 | C |
| ATOM | 2944 | CD2 | LEU | A | 364 | 17.084 | −28.618 | −5.714 | 1.00 | 33.75 | C |
| ATOM | 2945 | C | LEU | A | 364 | 18.415 | −32.639 | −5.987 | 1.00 | 38.64 | C |
| ATOM | 2946 | O | LEU | A | 364 | 18.444 | −33.374 | −6.963 | 1.00 | 45.74 | O |
| ATOM | 2947 | N | GLY | A | 365 | 18.512 | −33.085 | −4.736 | 1.00 | 36.04 | N |
| ATOM | 2948 | CA | GLY | A | 365 | 18.899 | −34.463 | −4.444 | 1.00 | 36.48 | C |
| ATOM | 2949 | C | GLY | A | 365 | 20.391 | −34.634 | −4.632 | 1.00 | 38.38 | C |
| 4TOM | 2950 | O | GLY | A | 365 | 20.842 | −35.322 | −5.560 | 1.00 | 42.76 | O |
| ATOM | 2951 | N | ALA | A | 366 | 21.146 | −34.008 | −3.733 | 1.00 | 38.75 | N |
| ATOM | 2352 | CA | ALA | A | 366 | 22.590 | −33.572 | −5.823 | 1.00 | 36.89 | C |
| ATOM | 2953 | CB | ALA | A | 366 | 23.027 | −32.609 | −3.094 | 1.00 | 36.10 | C |
| ATOM | 2954 | C | ALA | A | 366 | 23.372 | −35.050 | −3.273 | 1.00 | 39.85 | C |
| ATOM | 2955 | O | ALA | A | 366 | 24.631 | −35.042 | −3.345 | 1.00 | 36.59 | O |
| ATOM | 2956 | N | GLY | A | 367 | 22.669 | −36.029 | −2.687 | 1.00 | 35.93 | N |
| ATOM | 2957 | CA | GLY | A | 367 | 23.357 | −37.229 | −2.187 | 1.00 | 37.34 | C |
| ATOM | 2958 | C | GLY | A | 367 | 24.539 | −36.831 | −1.316 | 1.00 | 40.07 | C |
| ATOM | 2959 | O | GLY | A | 367 | 24.395 | −35.969 | −0.446 | 1.00 | 38.79 | O |
| ATOM | 2960 | N | ARG | A | 368 | 25.714 | −37.412 | −1.558 | 1.00 | 40.53 | N |
| ATOM | 2961 | CA | ARG | A | 368 | 26.907 | −37.011 | −0.778 | 1.00 | 44.28 | C |
| ATOM | 2962 | CS | ARG | A | 368 | 27.821 | −38.216 | −0.534 | 1.00 | 40.12 | C |
| ATOM | 2963 | CB | ARG | A | 368 | 27.225 | −39.290 | 0.356 | 1.00 | 41.42 | C |
| ATOM | 2964 | CG | ARG | A | 368 | 27.753 | −40.628 | −0.106 | 1.00 | 39.32 | C |
| ATOM | 2965 | NE | ARG | A | 368 | 28.614 | −41.251 | 0.873 | 1.00 | 40.28 | N |
| ATOM | 2966 | CZ | ARG | A | 368 | 29.710 | −41.937 | 0.583 | 1.00 | 40.01 | C |
| ATOM | 2967 | NH1 | ARG | A | 368 | 30.110 | −42.068 | −0.670 | 1.00 | 37.53 | N |
| ATOM | 2968 | NH2 | ARG | A | 368 | 30.413 | −42.476 | 1.568 | 1.00 | 43.39 | N |
| ATOM | 2969 | C | ARG | A | 368 | 27.740 | −35.834 | −1.351 | 1.00 | 47.19 | C |
| ATOM | 2970 | O | ARG | A | 368 | 28.847 | −35.566 | −0.869 | 1.00 | 52.32 | O |
| ATOM | 2971 | N | GLY | A | 369 | 27.233 | −35.171 | −2.389 | 1.00 | 46.64 | N |
| ATOM | 2972 | CA | GLY | A | 369 | 27.870 | −33.972 | −2.944 | 1.00 | 52.56 | C |
| ATOM | 2973 | C | GLY | A | 369 | 28.620 | −33.969 | −4.289 | 1.00 | 53.49 | C |
| ATOM | 2974 | O | GLY | A | 369 | 29.086 | −32.904 | −4.720 | 1.00 | 54.42 | O |
| ATOM | 2975 | N | PRO | A | 370 | 28.742 | −35.130 | −4.975 | 1.00 | 52.29 | N |
| ATOM | 2976 | CA | PRO | A | 370 | 29.592 | −35.077 | −6.171 | 1.00 | 50.98 | C |
| ATOM | 2977 | CB | PRO | A | 370 | 29.520 | −36.511 | −6.716 | 1.00 | 47.24 | C |
| ATOM | 2978 | CG | PRO | A | 370 | 28.710 | −37.017 | −6.228 | 1.00 | 48.09 | C |
| ATOM | 2979 | CD | PRO | A | 370 | 28.086 | −36.443 | −4.849 | 1.00 | 50.30 | C |
| ATOM | 2980 | C | PRO | A | 370 | 29.101 | −34.053 | −7.204 | 1.00 | 46.71 | C |
| ATOM | 2981 | O | PRO | A | 370 | 29.912 | −33.459 | −7.894 | 1.00 | 50.64 | O |
| ATOM | 2982 | N | LEU | A | 371 | 27.795 | −33.844 | −7.288 | 1.00 | 45.77 | N |
| ATOM | 2983 | CA | LEU | A | 371 | 27.218 | −32.852 | −8.700 | 1.00 | 48.65 | C |
| ATOM | 2984 | CB | LEU | A | 371 | 23.712 | −33.085 | −8.389 | 1.00 | 45.65 | C |
| ATOM | 2985 | CG | LEU | A | 371 | 25.137 | −33.629 | −9.712 | 1.00 | 47.53 | C |
| ATOM | 2986 | CD1 | LEU | A | 371 | 25.865 | −34.818 | −10.295 | 1.00 | 45.35 | C |
| ATOM | 2987 | CD2 | LEU | A | 371 | 23.653 | −33.954 | −9.569 | 1.00 | 46.55 | C |
| ATOM | 2988 | C | LEU | A | 371 | 27.469 | −31.420 | −7.720 | 1.00 | 48.85 | C |
| ATOM | 2989 | O | LEU | A | 371 | 27.481 | −30.482 | −8.523 | 1.00 | 50.26 | O |
| ATOM | 2990 | N | VAL | A | 371 | 27.654 | −31.255 | −6.413 | 1.00 | 47.02 | N |
| ATOM | 2991 | CA | VAL | A | 372 | 28.029 | −29.958 | −5.846 | 1.00 | 48.45 | C |
| ATOM | 2992 | CB | VAL | A | 372 | 27.786 | −29.901 | −4.309 | 1.00 | 50.78 | C |
| ATOM | 2993 | CG1 | va1 | A | 372 | 28.255 | −28.582 | −3.727 | 1.00 | 48.77 | C |
| ATOM | 2994 | CG2 | VAL | A | 372 | 26.314 | −30.110 | −3.975 | 1.00 | 47.83 | C |
| ATOM | 2995 | C | VAL | A | 372 | 29.509 | −29.705 | −6.208 | 1.00 | 49.29 | C |
| ATOM | 2996 | O | VAL | A | 372 | 29.857 | −28.642 | −6.705 | 1.00 | 47.66 | O |
| ATOM | 2997 | N | ASN | A | 373 | 30.367 | −30.399 | −6.013 | 1.00 | 44.75 | N |
| ATOM | 2998 | CA | ASN | A | 373 | 31.724 | −30.568 | −6.481 | 1.00 | 46.36 | C |
| ATOM | 2999 | CB | ASN | A | 373 | 32.551 | −31.767 | −6.077 | 1.00 | 46.43 | C |
| ATOM | 3000 | CG | ASN | A | 373 | 32.833 | −31.772 | −4.607 | 1.00 | 49.38 | C |
| ATOM | 3001 | OD1 | ASN | A | 373 | 33.215 | −30.739 | −4.045 | 1.00 | 46.12 | O |
| ATOM | 3002 | ND2 | ASN | A | 373 | 32.611 | −32.921 | −3.953 | 1.00 | 52.96 | u |
| ATOM | 3O03 | C | ASN | A | 373 | 31.819 | −30.342 | −7.971 | 1.00 | 48.49 | C |
| ATOM | 3004 | O | ASN | A | 373 | 32.671 | −29.584 | −8.424 | 1.00 | 53.13 | O |
| ATOM | 3005 | N | ALA | A | 374 | 30.953 | −30.889 | −8.740 | 1.00 | 46.73 | N |
| ATOM | 3006 | CA | ALA | A | 374 | 31.046 | −30.847 | −10.178 | 1.00 | 45.82 | C |
| ATOM | 3007 | CB | ALA | A | 374 | 30.306 | −31.358 | −10.894 | 1.00 | 42.63 | C |
| ATOM | 3008 | C | ALA | A | 574 | 30.525 | −29.174 | −10.577 | 1.00 | 47.44 | C |
| ATOM | 3009 | O | ALA | A | 374 | 30.901 | −28.338 | −11.620 | 1.00 | 48.40 | O |
| ATOM | 3010 | N | SER | A | 375 | 29.685 | −28.587 | −9.734 | 1.00 | 45.28 | N |
| ATOM | 3011 | CA | SER | A | 375 | 29.735 | −27.530 | −10.000 | 1.00 | 47.83 | C |
| ATOM | 3012 | CB | SER | A | 375 | 28.013 | −27.173 | −9.168 | 1.00 | 46.07 | C |
| ATOM | 3013 | OG | SER | A | 375 | 26.871 | −27.326 | −9.672 | 1.00 | 42.49 | O |
| ATOM | 3014 | C | SER | A | 375 | 30.364 | −26.558 | −9.729 | 1.00 | 49.76 | C |
| ATOM | 3015 | O | SER | A | 375 | 30.626 | −25.556 | −10.524 | 1.00 | 53.56 | O |
| ATOM | 3016 | N | LEU | A | 376 | 31.047 | −26.770 | −8.613 | 1.00 | 52.37 | N |
| ATOM | 3017 | CA | LEU | A | 376 | 32.169 | −25.337 | −8.223 | 1.00 | 47.71 | C |
| ATOM | 3018 | CB | LEU | A | 376 | 32.713 | −26.147 | −6.851 | 1.00 | 46.18 | C |
| ATOM | 3019 | CG | LEU | A | 376 | 31.867 | −26.164 | −5.587 | 1.00 | 47.03 | C |
| ATOM | 3020 | CD1 | LEU | A | 376 | 32.574 | −26.112 | −4.401 | 1.00 | 45.21 | C |
| ATOM | 3021 | CD2 | LEU | A | 376 | 31.518 | −26.709 | −5.284 | 1.00 | 45.50 | C |
| ATOM | 3022 | C | LEU | A | 376 | 33.273 | −26.023 | −9.270 | 1.00 | 48.15 | C |
| ATOM | 3023 | O | LEU | A | 376 | 33.906 | −26.012 | −9.578 | 1.00 | 46.85 | O |
| ATOM | 3024 | N | ARG | A | 377 | 33.500 | −27.307 | −9.833 | 1.00 | 45.34 | N |
| ATOM | 3025 | CA | ARG | A | 377 | 34.578 | −27.130 | −10.825 | 1.00 | 47.91 | C |
| ATOM | 3026 | CB | ARG | A | 377 | 34.984 | −28.782 | −11.067 | 1.00 | 46.45 | C |
| ATOM | 3027 | CG | ARG | A | 377 | 35.826 | −29.114 | −9.986 | 1.00 | 43.90 | C |
| ATOM | 3028 | CD | ARG | A | 377 | 36.297 | −30.787 | −10.441 | 1.00 | 50.38 | C |
| ATOM | 3029 | NE | ARG | A | 377 | 35.199 | −31.395 | −10.999 | 1.00 | 57.33 | N |
| ATOM | 3030 | CZ | ARG | A | 377 | 34.466 | −32.485 | −10.317 | 1.00 | 56.85 | C |
| ATOM | 3031 | NH1 | ARG | A | 377 | 34.691 | −32.710 | −9.029 | 1.00 | 56.37 | N |
| ATOM | 3032 | NH2 | ARG | A | 377 | 33.498 | −33.161 | −10.929 | 1.00 | 57.16 | N |
| ATOM | 3033 | C | ARG | A | 377 | 34.182 | −26.678 | −12.149 | 1.00 | 49.08 | C |
| ATOM | 3034 | O | ARG | A | 377 | 35.016 | −26.131 | −12.863 | 1.00 | 47.92 | O |
| ATOM | 3035 | N | ALA | A | 378 | 32.898 | −26.738 | −12.462 | 1.00 | 49.60 | N |
| ATOM | 3036 | CA | ALA | A | 378 | 32.391 | −26.105 | −13.661 | 1.00 | 52.32 | C |
| ATOM | 3037 | CB | ALA | A | 378 | 30.952 | −26.323 | −13.925 | 1.00 | 49.77 | C |
| ATOM | 3038 | C | ALA | A | 378 | 32.502 | −24.389 | −13.607 | 1.00 | 51.82 | C |
| ATOM | 3039 | O | ALA | A | 378 | 32.737 | −23.953 | −14.625 | 1.00 | 50.12 | O |
| ATOM | 3040 | N | ALA | A | 379 | 32.318 | −24.408 | −12.429 | 1.00 | 56.66 | N |
| ATOM | 3041 | CA | ALA | A | 379 | 32.354 | −22.352 | −12.310 | 1.00 | 61.55 | C |
| ATOM | 3042 | CB | ALA | A | 379 | 31.634 | −22.087 | −11.052 | 1.00 | 57.48 | C |
| ATOM | 3043 | C | ALA | A | 379 | 33.795 | −22.004 | −12.382 | 1.00 | 67.64 | C |
| ATOM | 3044 | O | ALA | A | 379 | 34.037 | −20.966 | −13.020 | 1.00 | 66.93 | O |
| ATOM | 3045 | N | LYS | A | 380 | 34.749 | −22.704 | −11.764 | 1.00 | 64.60 | N |
| ATOM | 3046 | CA | LYS | A | 380 | 36.149 | −22.292 | −11.889 | 1.00 | 73.60 | C |
| ATOM | 3047 | CB | LYS | A | 380 | 37.034 | −22.772 | −10.719 | 1.00 | 75.52 | C |
| ATOM | 3048 | CG | LYS | A | 380 | 37.151 | −24.275 | −10.526 | 1.00 | 89.09 | C |
| ATOM | 3049 | CD | LYS | A | 380 | 38.257 | −24.913 | −11.366 | 1.00 | 99.15 | C |
| ATOM | 3050 | CE | LYS | A | 380 | 38.268 | −26.432 | −11.193 | 1.00 | 101.19 | C |
| ATOM | 3051 | NZ | LYS | A | 380 | 39.172 | −27.144 | −12.138 | 1.00 | 97.76 | N |
| ATOM | 3052 | C | LYS | A | 380 | 36.742 | −22.652 | −13.256 | 1.00 | 72.59 | C |
| ATOM | 3053 | O | LYS | A | 380 | 37.842 | −22.223 | −13.584 | 1.00 | 77.11 | O |
| ATOM | 3054 | N | GLN | A | 381 | 36.008 | −23.424 | −14.051 | 1.00 | 67.96 | N |
| ATOM | 3055 | CA | GLN | A | 381 | 36.449 | −23.758 | −15.403 | 1.00 | 61.97 | C |
| ATOM | 3056 | CB | GLN | A | 381 | 36.014 | −25.173 | −15.786 | 1.00 | 65.43 | C |
| ATOM | 3057 | CG | GLN | A | 381 | 36.833 | −25.837 | −16.886 | 1.00 | 62.96 | C |
| ATOM | 3058 | CD | GLN | A | 381 | 36.087 | −26.981 | −17.566 | 1.00 | 67.14 | C |
| ATOM | 3059 | CE1 | GLN | A | 381 | 34.994 | −27.369 | −17.145 | 1.00 | 70.92 | O |
| ATOM | 3060 | NE2 | GLN | A | 381 | 36.675 | −27.523 | −18.626 | 1.00 | 64.82 | N |
| ATOM | 3061 | C | GLN | A | 381 | 35.841 | −22.744 | −16.350 | 1.00 | 55.73 | C |
| ATOM | 3062 | O | GLN | A | 381 | 36.419 | −22.399 | −17.369 | 1.00 | 50.66 | O |
| ATOM | 3063 | N | ALA | A | 382 | 34.667 | −22.248 | −15.991 | 1.00 | 55.81 | N |
| ATOM | 3064 | CA | ALA | A | 382 | 34.009 | −21.215 | −16.781 | 1.00 | 55.34 | C |
| ATOM | 3065 | CB | ALA | A | 382 | 32.497 | −21.267 | −16.558 | 1.00 | 48.62 | C |
| ATOM | 3066 | C | ALA | A | 382 | 34.549 | −19.845 | −16.407 | 1.00 | 57.59 | C |
| ATOM | 3067 | O | ALA | A | 382 | 34.186 | −18.840 | −17.030 | 1.00 | 59.98 | O |
| ATOM | 3068 | N | ASP | A | 383 | 35.414 | −19.821 | −13.387 | 1.00 | 64.97 | N |
| ATOM | 3069 | CA | ASP | A | 383 | 35.692 | −18.617 | −14.602 | 1.00 | 67.61 | C |
| ATOM | 3070 | CB | ASP | A | 383 | 36.854 | −17.794 | −15.193 | 1.00 | 75.49 | C |
| ATOM | 3071 | CG | ASP | A | 383 | 37.391 | −16.726 | −14.219 | 1.00 | 84.76 | C |
| ATOM | 3072 | OD1 | ASP | A | 383 | 36.594 | −15.912 | −13.689 | 1.00 | 86.90 | O |
| ATOM | 3073 | OD2 | ASP | A | 383 | 38.625 | −16.693 | −13.993 | 1.00 | 84.17 | O |
| ATOM | 3074 | C | ASP | A | 383 | 34.404 | −17.790 | −14.481 | 1.00 | 63.83 | C |
| ATOM | 3075 | O | ASP | A | 383 | 34.286 | −16.706 | −15.060 | 1.00 | 62.62 | O |
| ATOM | 3076 | N | ARG | A | 384 | 33.422 | −18.342 | −13.774 | 1.00 | 57.04 | N |
| ATOM | 3077 | CA | ARG | A | 384 | 32.225 | −17.579 | −13.408 | 1.00 | 58.13 | C |
| ATOM | 3078 | CD | ARG | A | 384 | 11.042 | −17.084 | −14.335 | 1.00 | 57.25 | C |
| ATOM | 3079 | CG | ARG | A | 384 | 31.248 | −17.559 | −15.810 | 1.00 | 59.32 | C |
| ATOM | 3080 | CD | ARG | A | 384 | 31.364 | −16.063 | −16.058 | 1.00 | 58.15 | C |
| ATOM | 3081 | NE | ARG | A | 384 | 31.235 | −15.701 | −17.472 | 1.00 | 59.70 | N |
| ATOM | 3082 | CZ | ARG | A | 384 | 32.191 | −15.838 | −18.397 | 1.00 | 61.30 | C |
| ATOM | 3083 | NH1 | ARG | A | 384 | 33.381 | −16.371 | −18.111 | 1.00 | 55.31 | N |
| ATOM | 3084 | NH2 | ARG | A | 384 | 31.939 | −15.460 | −19.636 | 1.00 | 61.65 | N |
| ATOM | 3085 | C | ARG | A | 384 | 31.856 | −17.831 | −11.945 | 1.00 | 58.88 | C |
| ATOM | 3086 | O | ARG | A | 384 | 32.328 | −18.793 | −11.323 | 1.00 | 59.93 | O |
| ATOM | 3087 | N | ARG | A | 385 | 31.018 | −16.964 | −11.396 | 1.00 | 61.65 | N |
| ATOM | 3088 | CA | ARG | A | 385 | 30.725 | −17023 | −9.978 | 1.00 | 65.50 | C |
| ATOM | 3089 | CB | ARG | A | 385 | 30.930 | −15.640 | −9.333 | 1.00 | 72.61 | C |
| ATOM | 3090 | CG | ARG | A | 385 | 32.397 | −15.255 | −9.171 | 1.00 | 74.63 | C |
| ATOM | 3091 | CD | ARG | A | 385 | 32.604 | −14.290 | −8.009 | 1.00 | 77.58 | C |
| ATOM | 3092 | NE | ARG | A | 385 | 31.811 | −13.049 | −8.170 | 1.00 | 76.00 | N |
| ATOM | 3093 | CZ | ARG | A | 385 | 30.992 | −12.546 | −7.274 | 1.00 | 74.78 | C |
| ATOM | 3094 | NH1 | ARG | A | 385 | 30.363 | −11.404 | −7.535 | 1.00 | 72.34 | N |
| ATOM | 3095 | NH2 | ARG | A | 385 | 30.775 | −13.163 | −6.118 | 1.00 | 74.37 | N |
| ATOM | 3096 | C | ARG | A | 385 | 29.333 | −17.582 | −9.688 | 1.00 | 65.26 | C |
| ATOM | 3097 | O | ARG | A | 385 | 28.346 | −17.218 | −10.345 | 1.00 | 65.36 | O |
| ATOM | 3098 | N | ILE | A | 386 | 29.270 | −18.478 | −8.699 | 1.00 | 62.06 | N |
| ATOM | 3099 | CA | ILE | A | 386 | 28.018 | −19.161 | −8.344 | 1.00 | 56.06 | C |
| ATOM | 3100 | CB | ILE | A | 386 | 27.960 | −20.579 | −8.943 | 1.00 | 53.19 | C |
| ATOM | 3101 | CG1 | ILE | A | 386 | 29.224 | −21.367 | −8.563 | 1.00 | 55.06 | C |
| ATOM | 3102 | CD1 | ILE | A | 386 | 29.005 | −22.850 | −8.340 | 1.00 | 55.71 | C |
| ATOM | 3103 | CG2 | ILE | A | 386 | 27.779 | −20.493 | −10.451 | 1.00 | 50.14 | C |
| ATOM | 3104 | C | ILE | A | 386 | 27.726 | −19.266 | −6.844 | 1.00 | 53.96 | C |
| ATOM | 3105 | O | ILE | A | 386 | 28.625 | −19.467 | −6.025 | 1.00 | 55.13 | O |
| ATOM | 3106 | N | LYS | A | 387 | 26.453 | −19.116 | −6.500 | 1.00 | 50.32 | N |
| ATOM | 3107 | CA | LYS | A | 387 | 25.970 | −19.496 | −5.183 | 1.00 | 50.43 | C |
| ATOM | 3108 | CB | LYS | A | 387 | 24.949 | −18.482 | −4.667 | 1.00 | 54.20 | C |
| ATOM | 3109 | CG | LYS | A | 387 | 24.691 | −18.526 | −3.166 | 1.00 | 54.30 | C |
| ATOM | 3110 | CD | LYS | A | 387 | 23.517 | −17.617 | −2.838 | 1.00 | 58.21 | C |
| ATOM | 3111 | CE | LYS | A | 387 | 23.543 | −17.188 | −1.384 | 1.00 | 65.05 | C |
| ATOM | 3112 | NZ | LYS | A | 387 | 22.577 | −16.073 | −1.174 | 1.00 | 69.55 | N |
| ATOM | 3113 | C | LYS | A | 387 | 25.319 | −20.868 | −5.328 | 1.00 | 49.22 | C |
| ATOM | 3114 | O | LYS | A | 387 | 24.603 | −21.106 | −6.298 | 1.00 | 50.67 | O |
| ATOM | 3115 | N | LEU | A | 388 | 25.551 | −21.752 | −4.358 | 1.00 | 47.05 | N |
| ATOM | 3116 | CA | LEU | A | 388 | 25.048 | −23.112 | −4.419 | 1.00 | 44.72 | C |
| ATOM | 3117 | CB | LEU | A | 388 | 26.212 | −24.093 | −4.336 | 1.00 | 44.93 | C |
| ATOM | 3118 | CG | LEU | A | 388 | 26.860 | −24.436 | −5.664 | 1.00 | 45.42 | C |
| ATOM | 3119 | CD1 | LEU | A | 388 | 28.193 | −25.112 | −5.410 | 1.00 | 42.22 | C |
| ATOM | 3120 | CD2 | LEU | A | 388 | 25.937 | −25.311 | −6.513 | 1.00 | 45.01 | C |
| ATOM | 3121 | C | LEU | A | 388 | 24.085 | −23.446 | −3.306 | 1.00 | 45.41 | C |
| ATOM | 3122 | O | LEU | A | 388 | 24.340 | −23.123 | −2.139 | 1.00 | 44.87 | O |
| ATOM | 3123 | N | TYR | A | 389 | 23.005 | −24.140 | −3.656 | 1.00 | 45.06 | N |
| ATOM | 3124 | CA | TYR | A | 389 | 22.162 | −24.306 | −2.649 | 1.00 | 42.18 | C |
| ATOM | 3125 | CB | TYR | A | 389 | 20.741 | −24.319 | −2.703 | 1.00 | 44.53 | C |
| ATOM | 3126 | CG | TYR | A | 389 | 20.538 | −22.399 | −2.279 | 1.00 | 51.50 | C |
| ATOM | 3127 | CD1 | TYR | A | 389 | 20.912 | −21.848 | −3.114 | 1.00 | 53.39 | C |
| ATOM | 3128 | CE1 | TYR | A | 389 | 20.694 | −20.533 | −2.748 | 1.00 | 55.05 | C |
| ATOM | 3129 | CZ | TYR | A | 389 | 20.094 | −20.253 | −1.535 | 1.00 | 56.87 | C |
| ATOM | 3130 | OH | TYR | A | 389 | 19.896 | −18.943 | −1.203 | 1.00 | 55.79 | O |
| ATOM | 3131 | CE2 | TYR | A | 389 | 19.697 | −21.275 | −0.680 | 1.00 | 55.15 | C |
| ATOM | 3132 | CD2 | TYR | A | 389 | 19.917 | −22.593 | −1.061 | 1.00 | 55.03 | C |
| ATOM | 3133 | C | TYR | A | 389 | 22.177 | −26.306 | −2.875 | 1.00 | 42.75 | C |
| ATOM | 3134 | O | TYR | A | 389 | 21.986 | −26.772 | −4.010 | 1.00 | 42.70 | O |
| ATOM | 3135 | N | ALA | A | 390 | 22.398 | −27.063 | −1.800 | 1.00 | 39.75 | N |
| ATOM | 3136 | CA | ALA | A | 390 | 22.426 | −28.520 | −1.886 | 1.00 | 39.40 | C |
| ATOM | 3137 | CB | ALA | A | 390 | 23.761 | −29.971 | −1.407 | 1.00 | 33.92 | C |
| ATOM | 3138 | C | ALA | A | 390 | 21.262 | −29.141 | −1.117 | 1.00 | 39.36 | C |
| ATOM | 3139 | O | ALA | A | 390 | 21.353 | −29.400 | 0.083 | 1.00 | 42.11 | O |
| ATOM | 3140 | N | VAL | A | 391 | 20.168 | −20.359 | −1.823 | 1.00 | 39.41 | N |
| ATOM | 3141 | CA | VAL | A | 391 | 18.956 | −29.336 | −1.235 | 1.00 | 39.55 | C |
| ATOM | 3142 | CB | VAL | A | 391 | 17.735 | −29.416 | −1.979 | 1.00 | 38.64 | C |
| ATOM | 3143 | CG1 | VAL | A | 391 | 16.468 | −29.915 | −1.313 | 1.00 | 41.82 | C |
| ATOM | 3144 | CG2 | VAL | A | 391 | 17.788 | −27.903 | −2.008 | 1.00 | 40.28 | C |
| ATOM | 3145 | C | VAL | A | 391 | 18.968 | −31.461 | −1.306 | 1.00 | 39.67 | C |
| ATOM | 3146 | O | VAL | A | 391 | 19.143 | −32.032 | −2.383 | 1.00 | 43.68 | O |
| ATOM | 3147 | N | GLU | A | 392 | 18.785 | −32.108 | −0.158 | 1.00 | 38.84 | N |
| ATOM | 3148 | CA | GLU | A | 392 | 18.816 | −33.560 | −0.043 | 1.00 | 38.28 | C |
| ATOM | 3149 | CB | GLU | A | 392 | 20.228 | −34.022 | 0.336 | 1.00 | 35.45 | C |
| ATOM | 3150 | CG | GLU | A | 392 | 20.430 | −35.525 | 0.486 | 1.00 | 36.23 | C |
| ATOM | 3151 | CD | GLU | A | 392 | 19.734 | −36.377 | −0.579 | 1.00 | 37.59 | C |
| ATOM | 3152 | OE1 | GLU | A | 392 | 20.027 | −36.269 | −1.780 | 1.00 | 37.57 | O |
| ATOM | 3153 | OE2 | GLU | A | 392 | 10.891 | −37.196 | −0.199 | 1.00 | 39.23 | O |
| ATOM | 3154 | C | GLU | A | 392 | 17.819 | −33.973 | 1.014 | 1.00 | 39.21 | C |
| ATOM | 3155 | O | GLU | A | 392 | 17.753 | −33.362 | 2.081 | 1.00 | 41.57 | O |
| ATOM | 3156 | N | LYS | A | 393 | 17.025 | −35.001 | 0.734 | 1.00 | 42.94 | N |
| ATOM | 3157 | CA | LYS | A | 393 | 15.984 | −35.399 | 1.698 | 1.00 | 39.87 | C |
| ATOM | 3158 | CB | LYS | A | 393 | 14.652 | −35.205 | 1.010 | 1.00 | 38.49 | C |
| ATOM | 3159 | CG | LYS | A | 393 | 14.698 | −36.808 | −0.015 | 1.00 | 39.69 | C |
| ATOM | 3160 | CD | LYS | A | 393 | 14.410 | −38.180 | 0.537 | 1.00 | 39.10 | C |
| ATOM | 3161 | CE | LYS | A | 393 | 14.700 | −39.282 | −0.461 | 1.00 | 40.74 | C |
| ATOM | 3162 | NZ | LYS | A | 393 | 14.620 | −40.61 | 0.169 | 1.00 | 43.22 | N |
| ATOM | 3163 | C | LYS | A | 393 | 16.454 | −38.545 | 2.571 | 1.00 | 38.12 | C |
| ATOM | 3164 | O | LYS | A | 393 | 15.941 | −36.740 | 3.656 | 1.00 | 41.19 | O |
| ATOM | 3165 | N | ASN | A | 394 | 17.453 | −37.285 | 2.111 | 1.00 | 37.63 | N |
| ATOM | 3166 | CA | ASN | A | 394 | 17.993 | −38.388 | 2.898 | 1.00 | 35.27 | C |
| ATOM | 3167 | CB | ASN | A | 394 | 18.782 | −39.142 | 2.007 | 1.00 | 30.43 | C |
| ATOM | 3168 | CG | ASN | A | 394 | 19.413 | −40.501 | 2.760 | 1.00 | 29.91 | C |
| ATOM | 3169 | OD1 | ASN | A | 394 | 19.344 | −40.608 | 3.991 | 1.00 | 33.20 | O |
| ATOM | 3170 | ND2 | ASN | A | 394 | 20.057 | −41.176 | 2.013 | 1.00 | 29.16 | N |
| ATOM | 3171 | C | ASN | A | 394 | 18.846 | −37.782 | 3.998 | 1.00 | 38.78 | C |
| ATOM | 3172 | C | ASN | A | 394 | 19.986 | −37.402 | 3.755 | 1.00 | 43.89 | O |
| ATOM | 3173 | N | PRO | A | 395 | 18.291 | −37.688 | 5.223 | 1.00 | 41.67 | N |
| ATOM | 3174 | CA | PRO | A | 395 | 19.004 | −37.044 | 6.328 | 1.00 | 39.08 | C |
| ATOM | 3175 | CB | PRO | A | 395 | 18.054 | −37.209 | 7.509 | 1.00 | 38.69 | C |
| ATOM | 3176 | CG | PRO | A | 395 | 17.210 | −38.384 | 7.143 | 1.00 | 40.21 | C |
| ATOM | 3177 | CD | PRO | A | 395 | 17.023 | −38.297 | 5.669 | 1.00 | 38.20 | C |
| ATOM | 3178 | C | PRO | A | 395 | 20.349 | −37.696 | 6.644 | 1.00 | 39.19 | C |
| ATOM | 3179 | C | PRO | A | 395 | 21.215 | −37.049 | 7.208 | 1.00 | 40.77 | O |
| ATOM | 3180 | N | ASN | A | 396 | 20.524 | −38.958 | 6.290 | 1.00 | 41.20 | N |
| ATOM | 3181 | CA | ASN | A | 396 | 21.804 | −39.632 | 6.525 | 1.00 | 42.75 | C |
| ATOM | 3182 | CB | ASN | A | 396 | 21.647 | −41.156 | 6.407 | 1.00 | 41.43 | C |
| ATOM | 3183 | CG | ASN | A | 396 | 20.470 | −41.695 | 7.223 | 1.00 | 40.46 | C |
| ATOM | 3184 | CG1 | ASN | A | 396 | 20.576 | −41.919 | 8.425 | 1.00 | 40.75 | O |
| ATOM | 3185 | ND2 | ASN | A | 396 | 19.338 | −41.873 | 6.572 | 1.00 | 38.63 | N |
| ATOM | 3186 | C | ASN | A | 396 | 22.881 | −39.111 | 5.562 | 1.00 | 47.00 | C |
| ATOM | 3187 | O | ASN | A | 396 | 24.042 | −39.011 | 5.917 | 1.00 | 49.01 | O |
| ATOM | 3188 | N | ALA | A | 397 | 22.488 | −38.781 | 4.337 | 1.00 | 49.24 | N |
| ATOM | 3189 | CA | ALA | A | 397 | 23.419 | −38.204 | 3.380 | 1.00 | 47.16 | C |
| ATOM | 3190 | CB | ALA | A | 397 | 22.864 | −38.307 | 1.967 | 1.00 | 48.95 | C |
| ATOM | 3191 | C | ALA | A | 397 | 23.649 | −36.745 | 3.763 | 1.00 | 47.94 | C |
| ATOM | 3192 | O | ALA | A | 397 | 24.786 | −36.262 | 3.703 | 1.00 | 46.36 | O |
| ATOM | 3193 | N | VAL | A | 398 | 22.569 | −36.056 | 4.162 | 1.00 | 41.61 | N |
| ATOM | 3194 | CA | VAL | A | 398 | 22.672 | −34.723 | 4.736 | 1.00 | 39.22 | C |
| ATOM | 3195 | CB | VAL | A | 398 | 21.326 | −34.198 | 5.272 | 1.00 | 39.45 | C |
| ATOM | 3196 | CG1 | VAL | A | 398 | 21.536 | −32.958 | 6.129 | 1.00 | 35.70 | C |
| ATOM | 3197 | CG2 | VAL | A | 398 | 20.396 | −33.842 | 4.112 | 1.00 | 38.84 | C |
| ATOM | 3198 | C | VAL | A | 398 | 23.730 | −34.612 | 5.827 | 1.00 | 39.54 | C |
| ATOM | 3199 | O | VAL | A | 398 | 24.241 | −33.530 | 6.075 | 1.00 | 41.07 | O |
| ATOM | 3200 | N | VAL | A | 399 | 24.052 | −35.718 | 6.488 | 1.00 | 42.31 | N |
| ATOM | 3201 | CA | VAL | A | 399 | 25.198 | −35.741 | 7.406 | 1.00 | 44.63 | C |
| ATOM | 3202 | CB | VAL | A | 399 | 25.251 | −37.062 | 8.223 | 1.00 | 43.69 | C |
| ATOM | 3203 | CG1 | VAL | A | 399 | 26.568 | −37.222 | 8.991 | 1.00 | 40.62 | C |
| ATOM | 3204 | CG2 | VAL | A | 399 | 24.057 | −37.112 | 9.169 | 1.00 | 29.77 | C |
| ATOM | 3205 | C | VAL | A | 399 | 26.508 | −35.484 | 6.648 | 1.00 | 45.45 | C |
| ATOM | 3206 | O | VAL | A | 399 | 27.282 | −34.604 | 7.020 | 1.00 | 55.19 | O |
| ATOM | 3207 | N | THR | A | 400 | 26.743 | −36.244 | 5.588 | 1.00 | 44.52 | N |
| ATOM | 3208 | CA | THR | A | 400 | 27.945 | −36.122 | 4.764 | 1.00 | 44.99 | C |
| ATOM | 3209 | C3 | THR | A | 400 | 27.884 | −37.154 | 3.634 | 1.00 | 44.07 | C |
| ATOM | 3210 | OG1 | THR | A | 400 | 27.239 | −38.329 | 4.126 | 1.00 | 45.89 | O |
| ATOM | 3211 | CG2 | THR | A | 400 | 29.269 | −37.521 | 3.118 | 1.00 | 41.96 | C |
| ATOM | 3212 | C | THR | A | 400 | 28.068 | −34.707 | 4.159 | 1.00 | 47.60 | C |
| ATOM | 3213 | O | THR | A | 400 | 29.122 | −34.084 | 4.215 | 1.00 | 44.46 | O |
| ATOM | 3214 | N | LEU | A | 401 | 26.966 | −34.208 | 3.609 | 1.00 | 47.73 | N |
| ATOM | 3215 | CA | LEU | A | 401 | 26.923 | −32.900 | 2.991 | 1.00 | 45.49 | C |
| ATOM | 3216 | CB | LEU | A | 401 | 25.518 | −32.612 | 2.454 | 1.00 | 42.80 | C |
| ATOM | 3217 | CG | LEU | A | 401 | 24.966 | −33.377 | 1.251 | 1.00 | 41.82 | C |
| ATOM | 3218 | CD1 | LEU | A | 401 | 23.536 | −32.946 | 0.958 | 1.00 | 39.32 | C |
| ATOM | 3219 | CD2 | LEU | A | 401 | 25.836 | −33.125 | 0.042 | 1.00 | 40.74 | C |
| ATOM | 3220 | C | LEU | A | 401 | 27.301 | −31.817 | 3.979 | 1.00 | 49.67 | C |
| ATOM | 3221 | O | LEU | A | 401 | 28.101 | −30.941 | 3.646 | 1.00 | 50.64 | O |
| ATOM | 3222 | N | GLU | A | 402 | 26.711 | −31.872 | 5.181 | 1.00 | 47.14 | N |
| ATOM | 3223 | CA | GLU | A | 402 | 26.945 | −30.860 | 6.209 | 1.00 | 45.60 | C |
| ATOM | 3224 | CB | GLU | A | 402 | 25.898 | −30.911 | 7.326 | 1.00 | 42.93 | C |
| ATOM | 3225 | CG | GLU | A | 402 | 24.630 | −30.180 | 6.919 | 1.00 | 47.09 | C |
| ATOM | 3226 | CD | GLU | A | 402 | 23.510 | −30.161 | 7.949 | 1.00 | 51.72 | C |
| ATOM | 3227 | OE1 | GLU | A | 402 | 23.434 | −31.038 | 8.845 | 1.00 | 56.58 | O |
| ATOM | 3228 | OE2 | GLU | A | 402 | 22.662 | −29.257 | 7.835 | 1.00 | 50.28 | O |
| ATOM | 3229 | C | GLU | A | 402 | 28.377 | −30.903 | 6.746 | 1.00 | 46.64 | C |
| ATOM | 3230 | O | GLU | A | 402 | 28.937 | −29.871 | 7.079 | 5.00 | 45.33 | O |
| ATOM | 3231 | N | ASN | A | 403 | 28.976 | −32.088 | 6.790 | 1.00 | 48.01 | N |
| ATOM | 3232 | CA | ASN | A | 403 | 30.392 | −32.185 | 7.108 | 5.00 | 48.76 | C |
| ATOM | 3233 | CB | ASN | A | 403 | 30.773 | −33.597 | 7.536 | 1.00 | 48.65 | C |
| ATOM | 3234 | CG | ASN | A | 403 | 30.353 | −33.916 | 8.962 | 1.00 | 52.88 | C |
| ATOM | 3235 | OD1 | ASN | A | 403 | 31.074 | −33.628 | 9.922 | 1.00 | 49.56 | O |
| ATOM | 3236 | ND2 | ASN | A | 403 | 29.189 | −34.552 | 9.104 | 1.00 | 55.42 | N |
| ATOM | 3237 | C | ASN | A | 403 | 31.294 | −31.712 | 5.962 | 1.00 | 53.22 | C |
| ATOM | 3238 | O | ASN | A | 403 | 32.367 | −31.188 | 6.215 | 1.00 | 57.01 | O |
| ATOM | 3239 | N | TRP | A | 404 | 30.869 | −31.397 | 4.712 | 1.00 | 54.47 | N |
| ATOM | 3240 | CA | TRP | A | 404 | 31.589 | −31.323 | 3.571 | 1.00 | 53.62 | C |
| ATOM | 3241 | CB | TRP | A | 404 | 31.038 | −31.812 | 2.224 | 1.00 | 56.23 | C |
| ATOM | 3242 | CG | TRP | A | 404 | 31.891 | −32.852 | 1.552 | 1.00 | 62.03 | C |
| ATOM | 3243 | CD1 | TRP | A | 404 | 31.763 | −34.211 | 1.661 | 1.00 | 66.29 | C |
| ATOM | 3244 | NE1 | TRP | A | 404 | 32.721 | −34.845 | 0.907 | 1.00 | 66.43 | N |
| ATOM | 3245 | CE2 | TRP | A | 404 | 33.493 | −33.907 | 0.283 | 1.00 | 69.91 | C |
| ATOM | 3246 | CD2 | TRP | A | 404 | 32.995 | −32.629 | 0.662 | 1.00 | 65.26 | C |
| ATOM | 3247 | CE3 | TRP | A | 404 | 33.615 | −31.484 | 0.149 | 1.00 | 64.64 | C |
| ATOM | 3248 | CZ3 | TRP | A | 404 | 34.703 | −31.640 | −0.772 | 1.00 | 65.72 | C |
| ATOM | 3249 | CH2 | TRP | A | 404 | 35.176 | −32.921 | −1.074 | 1.00 | 66.95 | C |
| ATOM | 3250 | CZ2 | TRP | A | 404 | 34.589 | −34.059 | −0.584 | 1.00 | 69.19 | C |
| ATOM | 3251 | C | TRP | A | 404 | 31.547 | −29.798 | 3.607 | 1.00 | 52.62 | C |
| ATOM | 3252 | C | TRP | A | 404 | 32.546 | −29.150 | 3.303 | 1.00 | 53.07 | O |
| ATOM | 3253 | N | GLN | A | 405 | 30.402 | −29.228 | 3.976 | 1.00 | 49.94 | N |
| ATOM | 3254 | CA | GLN | A | 405 | 30.233 | −27.793 | 3.867 | 1.00 | 52.02 | C |
| ATOM | 3255 | CB | GLN | A | 405 | 28.764 | −27.393 | 3.638 | 1.00 | 52.61 | C |
| ATOM | 3256 | CG | GLN | A | 405 | 28.021 | −26.808 | 4.820 | 1.00 | 57.47 | C |
| ATOM | 3257 | CD | GLN | A | 405 | 28.404 | −25.366 | 5.134 | 1.00 | 58.05 | C |
| ATOM | 3258 | OE1 | GLN | A | 405 | 28.899 | −25.086 | 6.223 | 1.00 | 57.61 | O |
| ATOM | 3259 | NE2 | GLN | A | 405 | 28.168 | −24.450 | 4.192 | 1.00 | 57.59 | N |
| ATOM | 3260 | C | GLN | A | 405 | 30.904 | −27.027 | 5.005 | 1.00 | 55.50 | C |
| ATOM | 3261 | O | GLN | A | 405 | 31.254 | −25.864 | 4.835 | 1.00 | 57.09 | O |
| ATOM | 3262 | N | PHE | A | 406 | 31.106 | −27.581 | 6.143 | 1.00 | 55.52 | N |
| ATOM | 3263 | CA | PHE | A | 406 | 31.877 | −27.089 | 7.219 | 1.00 | 56.87 | C |
| ATOM | 3264 | CB | PHE | A | 406 | 31.479 | −27.580 | 8.580 | 1.00 | 61.88 | C |
| ATOM | 3265 | CG | PHE | A | 406 | 32.318 | −27.175 | 9.724 | 1.00 | 64.20 | C |
| ATOM | 3266 | CD1 | PHE | A | 406 | 32.017 | −25.954 | 10.346 | 1.00 | 67.00 | C |
| ATOM | 3267 | CE1 | PHE | A | 406 | 32.806 | −25.483 | 11.385 | 1.00 | 65.94 | C |
| ATOM | 3268 | CZ | PHE | A | 406 | 33.912 | −26.209 | 11.811 | 1.00 | 64.09 | C |
| ATOM | 3269 | CE2 | PHE | A | 406 | 34.225 | −27.413 | 11.198 | 1.00 | 66.81 | C |
| ATOM | 3270 | CD2 | PHE | A | 406 | 33.432 | −27.391 | 10.160 | 1.00 | 66.14 | C |
| ATOM | 3271 | C | PHE | A | 406 | 33.388 | −27.241 | 7.002 | 1.00 | 58.63 | C |
| ATOM | 3272 | O | PHE | A | 406 | 34.152 | −26.330 | 7.311 | 1.00 | 55.94 | O |
| ATOM | 3273 | N | GLU | A | 407 | 33.817 | −28.401 | 6.515 | 1.00 | 56.81 | N |
| ATOM | 3274 | CA | GLU | A | 407 | 35.238 | −28.201 | 6.422 | 1.00 | 55.38 | C |
| ATOM | 3275 | CB | GLU | A | 407 | 35.510 | −30.211 | 6.454 | 1.00 | 55.07 | C |
| ATOM | 3276 | CG | GLU | A | 407 | 35.027 | −30.950 | 7.697 | 1.00 | 57.90 | C |
| ATOM | 3277 | CD | GLU | A | 407 | 35.662 | −32.329 | 7.877 | 1.00 | 62.36 | C |
| ATOM | 3278 | OE1 | GLU | A | 407 | 36.270 | −32.371 | 6.926 | 1.00 | 63.11 | O |
| ATOM | 3279 | OE2 | GLU | A | 407 | 35.570 | −32.881 | 8.997 | 1.00 | 66.55 | O |
| ATOM | 3280 | C | GLU | A | 407 | 35.865 | −28.116 | 5.166 | 1.00 | 57.19 | C |
| ATOM | 3281 | O | GLU | A | 407 | 37.055 | −27.857 | 5.142 | 1.00 | 57.33 | O |
| ATOM | 3282 | N | GLU | A | 408 | 35.066 | −27.918 | 4.122 | 1.00 | 61.49 | N |
| ATOM | 3283 | CA | GLU | A | 408 | 35.607 | −27.614 | 2.797 | 1.00 | 56.92 | C |
| ATOM | 3284 | CB | GLU | A | 408 | 35.601 | −28.860 | 1.910 | 1.00 | 58.79 | C |
| ATOM | 3285 | CG | GLU | A | 408 | 36.479 | −30.002 | 2.393 | 1.00 | 68.31 | C |
| ATOM | 3286 | CD | GLU | A | 408 | 37.963 | −29.151 | 2.193 | 1.00 | 72.57 | C |
| ATOM | 3287 | DE1 | GLU | A | 408 | 38.345 | −29.099 | 1.196 | 1.00 | 76.60 | O |
| ATOM | 3288 | OE2 | GLU | A | 408 | 38.754 | −30.221 | 3.031 | 1.00 | 74.84 | O |
| ATOM | 3289 | C | GLU | A | 408 | 34.867 | −26.497 | 2.082 | 1.00 | 54.93 | C |
| ATOM | 3290 | O | GLU | A | 408 | 35.429 | −25.455 | 1.808 | 1.00 | 55.42 | O |
| ATOM | 3291 | N | TRP | A | 409 | 33.604 | −26.715 | 1.758 | 1.00 | 55.22 | N |
| ATOM | 3292 | CA | TRP | A | 409 | 32.939 | −25.107 | 0.838 | 1.00 | 55.23 | C |
| ATOM | 3293 | CB | TRP | A | 409 | 31.548 | −26.325 | 0.432 | 1.00 | 54.39 | C |
| ATOM | 3294 | CG | TRP | A | 409 | 31.562 | −27.651 | −0.270 | 1.00 | 56.14 | C |
| ATOM | 3295 | CD1 | TRP | A | 409 | 32.565 | −28.265 | −1.054 | 1.00 | 57.64 | C |
| ATOM | 3296 | NE1 | TRP | A | 409 | 32.213 | −29.411 | −1.517 | 1.00 | 58.65 | N |
| ATOM | 3297 | CE2 | TRP | A | 409 | 30.967 | −29.725 | −1.036 | 1.00 | 57.91 | C |
| ATOM | 3298 | CD2 | TRP | A | 409 | 30.526 | −28.638 | −0.248 | 1.00 | 15.00 | C |
| ATOM | 3299 | CE3 | TRP | A | 409 | 29.262 | −25.707 | 0.356 | 1.00 | 52.36 | C |
| ATOM | 3300 | CZ3 | TRP | A | 409 | 28.492 | −29.847 | 0.167 | 1.00 | 53.35 | C |
| ATOM | 3301 | CH2 | TRP | A | 409 | 28.955 | −30.909 | −0.619 | 1.00 | 54.19 | C |
| ATOM | 3302 | CZ2 | TRP | A | 409 | 30.190 | −30.868 | −1.228 | 1.00 | 56.71 | C |
| ATOM | 3303 | C | TRP | A | 409 | 32.833 | −24.382 | 1.357 | 1.00 | 55.16 | C |
| ATOM | 3304 | O | TRP | A | 409 | 32.804 | −23.442 | 0.561 | 1.00 | 58.89 | O |
| ATOM | 3305 | N | GLY | A | 410 | 32.754 | −22.223 | 2.678 | 1.00 | 55.20 | N |
| ATOM | 3306 | CA | GLY | A | 410 | 32.409 | −22.932 | 3.282 | 1.00 | 52.78 | C |
| ATOM | 3307 | C | GLY | A | 410 | 31.029 | −22.497 | 2.826 | 1.00 | 53.28 | C |
| ATOM | 3308 | O | GLY | A | 410 | 30.287 | −23.296 | 2.238 | 1.00 | 51.86 | O |
| ATOM | 3309 | N | SER | A | 411 | 30.694 | −21.228 | 3.068 | 1.00 | 51.42 | N |
| ATOM | 3310 | CA | SER | A | 411 | 29.325 | −26.719 | 2.849 | 1.00 | 49.76 | C |
| ATOM | 3311 | CB | SER | A | 411 | 29.041 | −19.486 | 3.732 | 1.00 | 51.10 | C |
| ATOM | 3312 | OG | SER | A | 411 | 29.525 | −18.300 | 3.121 | 1.00 | 14.91 | O |
| ATOM | 3313 | C | SER | A | 411 | 28.958 | −20.448 | 1.380 | 1.00 | 47.87 | C |
| ATOM | 3314 | O | SER | A | 411 | 27.866 | −19.934 | 1.075 | 1.00 | 45.35 | O |
| ATOM | 3315 | N | GLN | A | 412 | 29.836 | −20.803 | 0.451 | 1.00 | 46.57 | N |
| ATOM | 3316 | CA | GLN | A | 412 | 29.382 | −20.807 | −0.933 | 1.00 | 12.28 | C |
| ATOM | 3317 | CB | GLN | A | 412 | 30.521 | −21.083 | −1.890 | 1.00 | 10.56 | C |
| ATOM | 3318 | CG | GLN | A | 412 | 30.200 | −20.594 | −3.289 | 1.00 | 12.17 | C |
| ATOM | 3319 | CD | GLN | A | 412 | 31.291 | −20.909 | −4.286 | 1.00 | 51.94 | C |
| ATOM | 3320 | CE1 | GLN | A | 412 | 32.267 | −21.603 | −3.975 | 1.00 | 54.92 | O |
| ATOM | 3321 | NE2 | GLN | A | 412 | 31.129 | −20.407 | −5.500 | 1.00 | 50.86 | N |
| ATOM | 3322 | C | GLN | A | 412 | 28.211 | −21.815 | −1.151 | 1.00 | 55.84 | C |
| ATOM | 3323 | O | GLN | A | 412 | 27.323 | −21.591 | −1.991 | 1.00 | 15.85 | O |
| ATOM | 3324 | N | VAL | A | 413 | 28.214 | −22.894 | −0.368 | 1.00 | 53.21 | N |
| ATOM | 3325 | CA | VAL | A | 413 | 27.155 | −23.902 | −0.402 | 1.00 | 53.99 | C |
| ATOM | 3326 | CB | VAL | A | 413 | 27.740 | −25.322 | −0.538 | 1.00 | 55.88 | C |
| ATOM | 3327 | CG1 | VAL | A | 413 | 26.627 | −26.330 | −0.813 | 1.00 | 55.68 | C |
| ATOM | 3328 | CG2 | VAL | A | 413 | 28.816 | −25.380 | −1.624 | 1.00 | 51.25 | C |
| ATOM | 3329 | C | VAL | A | 413 | 26.260 | −23.857 | 0.846 | 1.00 | 54.87 | C |
| ATOM | 3330 | C | VAL | A | 413 | 20.747 | −23.991 | 1.976 | 1.00 | 53.86 | O |
| ATOM | 3331 | N | THR | A | 414 | 24.958 | −23.663 | 0.626 | 1.00 | 53.69 | N |
| ATOM | 3332 | CA | THR | A | 414 | 23.933 | −23.787 | 1.672 | 1.00 | 53.46 | C |
| ATOM | 3333 | CB | THR | A | 414 | 22.819 | −22.734 | 1.491 | 1.00 | 13.17 | C |
| ATOM | 3334 | CG1 | THR | A | 414 | 23.400 | −21.432 | 1.485 | 1.00 | 60.06 | O |
| ATOM | 3335 | CG2 | THR | A | 414 | 21.769 | −22.808 | 2.607 | 1.00 | 48.39 | C |
| ATOM | 3336 | C | THR | A | 414 | 23.307 | −25.194 | 1.602 | 1.00 | 53.27 | C |
| ATOM | 3337 | C | THR | A | 414 | 22.554 | −25.506 | 0.665 | 1.00 | 50.61 | O |
| ATOM | 3338 | N | VAL | A | 415 | 23.607 | −26.033 | 2.593 | 1.00 | 49.64 | N |
| ATOM | 3339 | CA | VAL | A | 415 | 23.043 | −27.380 | 2.631 | 1.00 | 47.08 | C |
| ATOM | 3340 | CB | VAL | A | 415 | 23.905 | −28.345 | 3.454 | 1.00 | 42.93 | C |
| ATOM | 3341 | CG1 | VAL | A | 415 | 23.207 | −29.682 | 3.586 | 1.00 | 22.27 | C |
| ATOM | 3342 | CG2 | VAL | A | 415 | 25.237 | −28.543 | 2.762 | 1.00 | 44.78 | C |
| ATOM | 3343 | C | VAL | A | 415 | 21.622 | −27.331 | 3.168 | 1.00 | 45.72 | C |
| ATOM | 3344 | O | VAL | A | 415 | 21.396 | −26.808 | 4.258 | 1.00 | 46.00 | O |
| ATOM | 3345 | N | VAL | A | 416 | 20.669 | −27.832 | 2.380 | 1.00 | 26.22 | N |
| ATOM | 3346 | CA | VAL | A | 416 | 19.256 | −27.872 | 2.803 | 1.00 | 46.17 | C |
| ATOM | 3347 | CB | VAL | A | 416 | 18.313 | −27.110 | 1.864 | 1.00 | 44.35 | C |
| ATOM | 3348 | CG1 | VAL | A | 416 | 16.869 | −27.245 | 2.342 | 1.00 | 42.19 | C |
| ATOM | 3349 | CG2 | VAL | A | 416 | 18.720 | −25.647 | 1.790 | 1.00 | 46.55 | C |
| ATOM | 3350 | C | VAL | A | 416 | 18.761 | −29.298 | 2.969 | 1.00 | 47.51 | C |
| ATOM | 3351 | O | VAL | A | 416 | 18.735 | −30.088 | 2.018 | 1.00 | 45.62 | O |
| ATOM | 3352 | N | SER | A | 417 | 18.373 | −29.622 | 4.194 | 1.00 | 48.22 | N |
| ATOM | 3353 | CA | SER | A | 417 | 17.822 | −30.926 | 4.485 | 1.00 | 49.63 | C |
| ATOM | 3354 | CB | SER | A | 417 | 18.255 | −31.387 | 5.870 | 1.00 | 49.40 | C |
| ATOM | 3355 | OG | SER | A | 417 | 17.817 | −32.714 | 6.100 | 1.00 | 57.95 | O |
| ATOM | 3356 | C | SER | A | 417 | 16.306 | −30.863 | 4.391 | 1.00 | 48.37 | C |
| ATOM | 3357 | O | SER | A | 417 | 15.648 | −30.542 | 5.372 | 1.00 | 47.25 | O |
| ATOM | 3358 | N | SER | A | 418 | 15.770 | −31.129 | 3.197 | 1.00 | 48.01 | N |
| ATOM | 3359 | CA | SER | A | 418 | 14.322 | −31.301 | 3.003 | 1.00 | 49.17 | C |
| ATOM | 3360 | CB | SER | A | 418 | 13.564 | −29.979 | 3.177 | 1.00 | 50.40 | C |
| ATOM | 3361 | OG | SER | A | 418 | 13.456 | −29.301 | 1.948 | 1.00 | 50.16 | O |
| ATOM | 3362 | C | SER | A | 418 | 13.981 | −31.898 | 1.633 | 1.00 | 50.25 | C |
| ATOM | 3363 | O | SER | A | 418 | 14.855 | −32.109 | 0.787 | 1.00 | 50.42 | O |
| ATOM | 3364 | N | ASP | A | 419 | 12.696 | −32.167 | 1.432 | 1.00 | 45.08 | N |
| ATOM | 3365 | CA | ASP | A | 419 | 12.217 | −32.644 | 0.170 | 1.00 | 43.04 | C |
| ATOM | 3366 | CB | ASP | A | 419 | 10.852 | −33.305 | 0.353 | 1.00 | 43.05 | C |
| ATOM | 3367 | CG | ASP | A | 419 | 10.472 | −34.188 | −0.824 | 1.00 | 43.63 | C |
| ATOM | 3368 | OD1 | ASP | A | 419 | 10.234 | −33.683 | −1.946 | 1.00 | 41.65 | O |
| ATOM | 3369 | OD2 | ASP | A | 419 | 10.433 | −35.412 | −0.625 | 1.00 | 48.25 | O |
| ATOM | 3370 | C | ASP | A | 419 | 12.106 | −31.459 | −0.790 | 1.00 | 46.50 | C |
| ATOM | 3371 | O | ASP | A | 419 | 11.570 | −30.403 | −0.424 | 1.00 | 46.50 | O |
| ATOM | 3372 | N | MET | A | 420 | 12.567 | −31.635 | −2.026 | 1.00 | 41.97 | N |
| ATOM | 3373 | CA | MET | A | 420 | 12.504 | −30.541 | −2.987 | 1.00 | 39.77 | C |
| ATOM | 3374 | CB | MET | A | 420 | 13.266 | −30.866 | −4.264 | 1.00 | 38.64 | C |
| ATOM | 3375 | CG | MET | A | 420 | 12.601 | −31.918 | −5.136 | 1.00 | 41.52 | C |
| ATOM | 3376 | SD | MET | A | 420 | 13.403 | −32.183 | −6.733 | 1.00 | 40.88 | S |
| ATOM | 3377 | CE | MET | A | 420 | 14.897 | −33.068 | −6.205 | 1.00 | 34.57 | C |
| ATOM | 3378 | C | MET | A | 420 | 11.080 | −30.096 | −3.314 | 1.00 | 42.24 | C |
| ATOM | 3379 | O | MET | A | 420 | 10.899 | −28.973 | −3.758 | 1.00 | 43.88 | O |
| ATOM | 3380 | N | ARG | A | 421 | 10.080 | −30.966 | −3.114 | 1.00 | 42.78 | N |
| ATOM | 3381 | CA | ARG | A | 421 | 8.660 | −30.583 | −3.331 | 1.00 | 44.35 | C |
| ATOM | 3382 | CB | ARG | A | 421 | 7.733 | −31.799 | −3.462 | 1.00 | 44.20 | C |
| ATOM | 3383 | CG | ARG | A | 421 | 7.959 | −32.702 | −4.668 | 1.00 | 44.75 | C |
| ATOM | 3384 | CD | ARG | A | 421 | 8.476 | −34.034 | −4.193 | 1.00 | 43.95 | C |
| ATOM | 3385 | NE | ARG | A | 421 | 7.448 | −35.062 | −4.182 | 1.00 | 46.40 | N |
| ATOM | 3386 | CZ | ARG | A | 471 | 7.401 | −36.074 | −3.317 | 1.00 | 47.67 | C |
| ATOM | 3387 | NH1 | ARG | A | 421 | 6.441 | −36.983 | −3.409 | 1.00 | 54.69 | N |
| ATOM | 3388 | NH2 | ARG | A | 421 | 8.298 | −36.180 | −2.354 | 1.00 | 42.93 | N |
| ATOM | 3389 | C | ARG | A | 421 | 8.116 | −29.712 | −2.208 | 1.00 | 45.04 | C |
| ATOM | 3390 | O | ARG | A | 421 | 7.049 | −29.121 | −2.343 | 1.00 | 47.52 | O |
| ATOM | 3391 | N | GLU | A | 422 | 8.848 | −29.647 | −1.103 | 1.00 | 46.17 | N |
| ATOM | 3392 | CA | GLU | A | 422 | 8.386 | −29.004 | 0.120 | 1.00 | 53.17 | C |
| ATOM | 3393 | CB | GLU | A | 422 | 8.573 | −29.953 | 1.313 | 1.00 | 60.87 | C |
| ATOM | 3394 | CG | GLU | A | 422 | 7.680 | −31.181 | 1.292 | 1.00 | 69.35 | C |
| ATOM | 3395 | CD | GLU | A | 422 | 6.214 | −30.815 | 1.161 | 1.00 | 75.53 | C |
| ATOM | 3396 | OE1 | GLU | A | 422 | 5.702 | −30.076 | 2.032 | 1.00 | 81.39 | O |
| ATOM | 3397 | OE2 | GLU | A | 422 | 5.579 | −31.260 | 0.182 | 1.00 | 76.82 | O |
| ATOM | 3398 | C | GLU | A | 422 | 9.141 | −27.711 | 0.398 | 1.00 | 53.16 | C |
| ATOM | 3399 | O | GLU | A | 422 | 8.538 | −26.696 | 0.745 | 1.00 | 51.39 | O |
| ATOM | 3400 | N | TRP | A | 423 | 10.466 | −27.773 | 0.250 | 1.00 | 57.61 | N |
| ATOM | 3401 | CA | TRP | A | 423 | 11.364 | −26.651 | 0.507 | 1.00 | 55.46 | C |
| ATOM | 3402 | CB | TRP | A | 423 | 12.721 | −26.916 | −0.126 | 1.00 | 55.94 | C |
| ATOM | 3403 | CG | TRP | A | 423 | 13.779 | −25.942 | 0.269 | 1.00 | 58.11 | C |
| ATOM | 3404 | CD1 | TRP | A | 423 | 14.061 | −25.492 | 1.532 | 1.00 | 60.18 | C |
| ATOM | 3405 | NE1 | TRP | A | 423 | 15.123 | −24.515 | 1.497 | 1.00 | 61.21 | N |
| ATOM | 3406 | CE2 | TRP | A | 423 | 15.540 | −24.481 | 0.198 | 1.00 | 59.65 | N |
| ATOM | 3407 | CD2 | TRP | A | 423 | 14.717 | −25.310 | −0.602 | 1.00 | 57.05 | C |
| ATOM | 3408 | CE3 | TRP | A | 423 | 14.945 | −25.361 | −1.986 | 1.00 | 57.72 | C |
| ATOM | 3409 | CZ3 | TRP | A | 423 | 15.967 | −24.591 | −2.526 | 1.00 | 58.51 | C |
| ATOM | 3410 | CH2 | TRP | A | 423 | 16.759 | −23.764 | −1.704 | 1.00 | 62.37 | C |
| ATOM | 3411 | CZ2 | TRP | A | 423 | 16.561 | −23.700 | −0.342 | 1.00 | 59.17 | C |
| ATOM | 3412 | C | TRP | A | 423 | 10.804 | −25.117 | 0.035 | 1.00 | 57.17 | C |
| ATOM | 3413 | O | TRP | A | 423 | 10.414 | −25.166 | −1.124 | 1.00 | 55.66 | O |
| ATOM | 3414 | N | VAL | A | 424 | 10.720 | −24.165 | 0.960 | 1.00 | 56.65 | N |
| ATOM | 3415 | CA | VAL | A | 424 | 10.345 | −23.111 | 0.603 | 1.00 | 57.48 | C |
| ATOM | 3416 | CB | VAL | A | 424 | 9.438 | −22.145 | 1.671 | 1.00 | 60.17 | C |
| ATOM | 3417 | CGI | VAL | A | 424 | 9.275 | −20.356 | 1.403 | 1.00 | 58.39 | C |
| ATOM | 3418 | CG2 | VAL | A | 424 | 8.063 | −23.115 | 1.697 | 1.00 | 55.99 | C |
| ATOM | 3419 | C | VAL | A | 424 | 11.674 | −22.105 | 0.401 | 1.00 | 55.99 | C |
| ATOM | 3420 | O | VAL | A | 424 | 12.441 | −22.128 | 1.351 | 1.00 | 61.59 | O |
| ATOM | 3421 | N | ALA | A | 425 | 11.966 | −21.965 | −0.852 | 1.00 | 57.93 | N |
| ATOM | 3422 | CA | ALA | A | 425 | 13.283 | −21.447 | −1.222 | 1.00 | 62.21 | C |
| ATOM | 3423 | CB | ALA | A | 425 | 13.581 | −21.764 | −2.681 | 1.00 | 61.76 | C |
| ATOM | 3424 | C | ALA | A | 425 | 13.408 | −19.941 | −0.959 | 1.00 | 59.89 | C |
| ATOM | 3425 | O | ALA | A | 425 | 12.514 | −19.173 | −1.315 | 1.00 | 61.36 | O |
| ATOM | 3426 | H | PRO | A | 426 | 14.522 | −19.510 | −0.340 | 1.00 | 61.27 | N |
| ATOM | 3427 | CA | PRO | A | 426 | 14.675 | −19.081 | −0.013 | 1.00 | 62.93 | C |
| ATOM | 3428 | CB | PRO | A | 426 | 15.965 | −19.051 | 0.809 | 1.00 | 59.93 | C |
| ATOM | 3429 | CG | PRO | A | 426 | 16.749 | −19.221 | 0.310 | 1.00 | 60.09 | C |
| ATOM | 3430 | CD | PRO | A | 426 | 15.725 | −20.283 | 0.019 | 1.00 | 61.84 | C |
| ATOM | 3431 | C | PRO | A | 426 | 14.806 | −17.175 | −1.254 | 1.00 | 61.45 | C |
| ATOM | 3432 | O | PRO | A | 426 | 14.643 | −15.963 | −1.148 | 1.00 | 60.13 | O |
| ATOM | 3433 | N | GLU | A | 427 | 15.082 | −17.781 | −2.410 | 1.00 | 61.35 | N |
| ATOM | 3434 | CA | GLU | A | 427 | 15.261 | −17.089 | −3.689 | 1.00 | 56.98 | C |
| ATOM | 3435 | CB | GLU | A | 427 | 16.653 | −16.446 | −3.763 | 1.00 | 55.25 | C |
| ATOM | 3436 | CG | GLU | A | 427 | 17.815 | −11.375 | −3.442 | 1.00 | 53.86 | C |
| ATOM | 3437 | CD | GLU | A | 427 | 19.173 | −16.718 | −3.683 | 1.00 | 54.86 | C |
| ATOM | 3438 | OE1 | GLU | A | 427 | 19.243 | −15.814 | −4.539 | 1.00 | 54.54 | O |
| ATOM | 3439 | OE2 | GLU | A | 427 | 20.180 | −17.111 | −3.038 | 1.00 | 53.19 | O |
| ATOM | 3440 | C | GLU | A | 427 | 15.078 | −18.072 | −4.850 | 1.00 | 57.82 | C |
| ATOM | 3441 | O | GLU | A | 427 | 15.183 | −19.284 | −4.663 | 1.00 | 59.39 | O |
| ATOM | 3442 | N | LYS | A | 428 | 14.807 | −17.549 | −6.045 | 1.00 | 59.96 | N |
| ATOM | 3443 | CA | LYS | A | 428 | 14.681 | −18.371 | −7.255 | 1.00 | 55.05 | C |
| ATOM | 3444 | CB | LYS | A | 428 | 13.757 | −11.687 | −8.267 | 1.00 | 52.16 | C |
| ATOM | 3445 | CG | LYS | A | 428 | 12.351 | −17.450 | −7.700 | 1.00 | 57.69 | C |
| ATOM | 3446 | CD | LYS | A | 428 | 11.283 | −17.129 | −8.746 | 1.00 | 53.68 | C |
| ATOM | 3447 | CE | LYS | A | 428 | 9.909 | −17.414 | −8.162 | 1.00 | 61.39 | C |
| ATOM | 3448 | NZ | LYS | A | 428 | 8.766 | −16.755 | −8.862 | 1.00 | 63.16 | N |
| ATOM | 3449 | C | LYS | A | 428 | 16.057 | −18.749 | −7.846 | 1.00 | 35.12 | C |
| ATOM | 3450 | O | LYS | A | 428 | 17.070 | −18.108 | −7.554 | 1.00 | 56.23 | O |
| ATOM | 3451 | N | ALA | A | 429 | 16.108 | −15.817 | −8.635 | 1.00 | 52.61 | N |
| ATOM | 3452 | CA | ALA | A | 429 | 17.392 | −20.298 | −9.172 | 1.00 | 50.07 | C |
| ATOM | 3453 | CB | ALA | A | 429 | 17.569 | −21.789 | −8.924 | 1.00 | 46.85 | C |
| ATOM | 3454 | C | ALA | A | 429 | 17.566 | −20.000 | −10.649 | 1.00 | 49.71 | C |
| ATOM | 3455 | C | ALA | A | 429 | 16.582 | −19.920 | −11.412 | 1.00 | 45.38 | O |
| ATOM | 3456 | N | ASP | A | 430 | 16.031 | −19.041 | −11.034 | 1.00 | 47.51 | N |
| ATOM | 3457 | CA | ASP | A | 430 | 19.218 | −19.698 | −12.425 | 1.00 | 49.63 | C |
| ATOM | 3458 | CB | ASP | A | 430 | 20.631 | −19.111 | −12.498 | 1.00 | 57.80 | C |
| ATOM | 3459 | CG | ASP | A | 430 | 20.654 | −17.613 | −12.288 | 1.00 | 56.51 | C |
| ATOM | 3460 | CD1 | ASP | A | 430 | 20.318 | −16.890 | −13.259 | 1.00 | 50.79 | O |
| ATOM | 3461 | CD2 | ASP | A | 430 | 21.019 | −17.176 | −11.167 | 1.00 | 52.14 | O |
| ATOM | 3462 | C | ASP | A | 430 | 19.222 | −21.059 | −13.118 | 1.00 | 50.23 | C |
| ATOM | 3463 | C | ASP | A | 430 | 18.897 | −21.175 | −14.309 | 1.00 | 48.68 | O |
| ATOM | 3464 | N | ILE | A | 431 | 19.630 | −22.076 | −12.356 | 1.00 | 48.91 | N |
| ATOM | 3465 | CA | ILE | A | 431 | 19.808 | −23.431 | −12.837 | 1.00 | 46.08 | C |
| ATOM | 3466 | CB | ILE | A | 431 | 21.249 | −23.722 | −13.327 | 1.00 | 48.90 | C |
| ATOM | 3467 | CG1 | ILE | A | 431 | 21.835 | −22.576 | −14.191 | 1.00 | 45.38 | C |
| ATOM | 3468 | CD1 | ILE | A | 431 | 23.314 | −22.742 | −14.500 | 1.00 | 46.74 | C |
| ATOM | 3469 | CG2 | ILE | A | 431 | 21.317 | −25.108 | −13.989 | 1.00 | 42.93 | C |
| ATOM | 3470 | C | ILE | A | 431 | 19.607 | −24.389 | −11.680 | 1.00 | 48.51 | C |
| ATOM | 3471 | O | ILE | A | 431 | 20.213 | −24.226 | −10.606 | 1.00 | 42.81 | O |
| ATOM | 3472 | N | ILE | A | 432 | 18.766 | −25.395 | −11.916 | 1.00 | 45.48 | N |
| ATOM | 3473 | CA | ILE | A | 432 | 18.675 | −26.533 | −11.043 | 1.00 | 38.85 | C |
| ATOM | 3474 | CB | ILE | A | 432 | 17.208 | −26.861 | −10.737 | 1.00 | 43..70 | C |
| ATOM | 3475 | CG1 | ILE | A | 432 | 16.398 | −27.037 | −12.024 | 1.00 | 41.81 | C |
| ATOM | 3476 | CD1 | ILE | A | 432 | 14.895 | −27.023 | −11.822 | 1.00 | 42.49 | C |
| ATOM | 3477 | CG2 | ILE | A | 432 | 16.595 | −25.761 | −9.871 | 1.00 | 45.05 | C |
| ATOM | 3478 | C | ILE | A | 432 | 19.420 | −27.713 | −11.682 | 1.00 | 39.52 | C |
| ATOM | 3479 | O | ILE | A | 432 | 19.369 | −27.936 | −12.894 | 1.00 | 40.89 | O |
| ATOM | 3480 | N | VAL | A | 433 | 20.142 | −28.450 | −10.863 | 1.00 | 36.02 | N |
| ATOM | 3481 | CA | VAL | A | 433 | 20.846 | −29.624 | −11.311 | 1.00 | 37.68 | C |
| ATOM | 3482 | CB | VAL | A | 433 | 22.359 | −29.502 | −11.043 | 1.00 | 37.51 | C |
| ATOM | 3483 | CB1 | VAL | A | 433 | 23.077 | −30.773 | −11.428 | 1.00 | 39.74 | C |
| ATOM | 3484 | CB2 | VAL | A | 433 | 22.947 | −28.353 | −11.828 | 1.00 | 36.75 | C |
| ATOM | 3485 | c | VAL | A | 433 | 20.282 | −30.815 | −10.542 | 1.00 | 37.43 | C |
| ATOM | 3486 | O | VAL | A | 433 | 20.106 | −30.736 | −9.324 | 1.00 | 36.34 | O |
| ATOM | 3487 | N | SER | A | 434 | 19.970 | −31.901 | −11.246 | 1.00 | 35.12 | N |
| ATOM | 3488 | CA | SER | A | 434 | 19.446 | −33.104 | −10.575 | 1.00 | 34.08 | C |
| ATOM | 3489 | CB | SER | A | 434 | 17.902 | −33.099 | −10.479 | 1.00 | 33.11 | C |
| ATOM | 3490 | OG | SER | A | 434 | 17.285 | −33.266 | −11.749 | 1.00 | 31.71 | O |
| ATOM | 3491 | C | SER | A | 434 | 19.860 | −34.321 | −11.302 | 1.00 | 32.40 | C |
| ATOM | 3492 | O | SER | A | 434 | 20.032 | −34.287 | −12.507 | 1.00 | 36.25 | O |
| ATOM | 3493 | N | GLU | A | 135 | 20.002 | −35.405 | −10.558 | 1.00 | 36.15 | N |
| ATOM | 3494 | CA | GLU | A | 435 | 20.192 | −36.725 | −11.132 | 1.00 | 33.79 | C |
| ATOM | 3495 | CB | GLU | A | 435 | 21.644 | −37.160 | −10.928 | 1.00 | 33.50 | C |
| ATOM | 3496 | CG | GLU | A | 435 | 22.048 | −38.321 | −11.821 | 1.00 | 37.87 | C |
| ATOM | 3497 | CD | GLU | A | 435 | 21.439 | −39.648 | −11.384 | 1.00 | 43.18 | C |
| ATOM | 3498 | OE1 | GLU | A | 435 | 22.176 | −40.434 | −10.749 | 1.00 | 41.55 | O |
| ATOM | 3499 | OE2 | GLU | A | 435 | 20.223 | −39.901 | −11.666 | 1.00 | 42.31 | O |
| ATOM | 3500 | C | GLU | A | 435 | 19.195 | −37.707 | −10.468 | 1.00 | 31.78 | C |
| ATOM | 3501 | O | GLU | A | 435 | 19.512 | −38.438 | −9.517 | 1.00 | 27.62 | O |
| ATOM | 3522 | N | LEU | A | 436 | 17.968 | −37.706 | −10.959 | 1.00 | 34.36 | N |
| ATOM | 3503 | CA | LEU | A | 436 | 16.911 | −38.466 | −10.289 | 1.00 | 34.93 | C |
| ATOM | 3504 | CB | LEU | A | 436 | 15.789 | −37.518 | −9.934 | 1.00 | 37.03 | C |
| ATOM | 3505 | CG | LEU | A | 436 | 16.283 | −36.309 | −9.174 | 1.00 | 35.30 | C |
| ATOM | 3506 | CD1 | LEU | A | 436 | 15.148 | −35.305 | −9.069 | 1.00 | 30.46 | C |
| ATOM | 3507 | CD2 | LEU | A | 436 | 16.820 | −36.787 | −7.822 | 1.00 | 33.66 | C |
| ATOM | 3508 | C | LEU | A | 436 | 16.353 | −39.538 | −11.189 | 1.00 | 36.85 | C |
| ATOM | 3509 | O | LEU | A | 436 | 15.137 | −39.812 | −11.167 | 1.00 | 35.76 | O |
| ATOM | 3510 | N | LEU | A | 437 | 17.242 | −40.118 | −11.991 | 1.00 | 35.38 | N |
| ATOM | 3511 | CA | LEU | A | 437 | 16.843 | −40.871 | −13.152 | 1.00 | 36.33 | C |
| ATOM | 3512 | CB | LEU | A | 437 | 17.636 | −40.420 | −14.372 | 1.00 | 39.74 | C |
| ATOM | 3513 | CG | LEU | A | 437 | 17.392 | −39.026 | −14.941 | 1.00 | 44.82 | C |
| ATOM | 3514 | CD1 | LEU | A | 437 | 18.119 | −38.924 | −16.278 | 1.00 | 44.46 | C |
| ATOM | 3515 | CD2 | LEU | A | 437 | 15.905 | −38.729 | −15.109 | 1.00 | 43.81 | C |
| ATOM | 3516 | C | LEU | A | 437 | 17.102 | −42.325 | −12.939 | 1.00 | 36.50 | C |
| ATOM | 3517 | O | LEU | A | 437 | 18.158 | −42.696 | −12.416 | 1.00 | 37.27 | O |
| ATOM | 3518 | N | GLY | A | 438 | 16.153 | −43.151 | −13.381 | 1.00 | 32.53 | N |
| ATOM | 3519 | CA | GLY | A | 438 | 16.294 | −44.602 | −13.286 | 1.00 | 28.23 | C |
| ATOM | 3520 | C | GLY | A | 438 | 16.527 | −45.182 | −14.653 | 1.00 | 27.06 | C |
| ATOM | 3521 | O | GLY | A | 438 | 16.533 | −44.445 | −15.626 | 1.00 | 25.68 | O |
| ATOM | 3522 | N | SER | A | 439 | 16.671 | −46.504 | −14.737 | 1.00 | 27.86 | N |
| ATOM | 3523 | CA | SER | A | 439 | 17.078 | −47.135 | −15.991 | 1.00 | 29.53 | C |
| ATOM | 3524 | CB | SER | A | 439 | 17.393 | −48.619 | −15.821 | 1.00 | 29.29 | C |
| ATOM | 3525 | OG | SER | A | 439 | 18.269 | −48.858 | −14.734 | 1.00 | 29.81 | O |
| ATOM | 3526 | C | SER | A | 439 | 16.054 | −46.936 | −17.084 | 1.00 | 28.89 | C |
| ATOM | 3527 | O | SER | A | 439 | 16.363 | −47.147 | −18.259 | 1.00 | 28.58 | O |
| ATOM | 3528 | N | PHE | A | 440 | 14.851 | −46.508 | −16.706 | 1.00 | 29.34 | N |
| ATOM | 3529 | CA | PHE | A | 440 | 13.784 | −46.292 | −17.674 | 1.00 | 28.69 | C |
| ATOM | 3530 | CB | PHE | A | 440 | 12.586 | −47.162 | −17.292 | 1.00 | 28.88 | C |
| ATOM | 3531 | CG | PHE | A | 440 | 11.620 | −47.419 | −18.418 | 1.00 | 27.75 | C |
| ATOM | 3532 | CD1 | PHE | A | 440 | 12.071 | −47.863 | −19.672 | 1.00 | 28.40 | C |
| ATOM | 3533 | CE1 | PHE | A | 440 | 11.175 | −48.112 | −20.703 | 1.00 | 28.14 | C |
| ATOM | 3534 | CZ | PHE | A | 440 | 9.795 | −47.348 | −20.486 | 1.00 | 25.56 | C |
| ATOM | 3535 | CE2 | PHE | A | 440 | 9.344 | −47.541 | −19.252 | 1.00 | 25.16 | C |
| ATOM | 3536 | CD2 | PHE | A | 440 | 10.248 | −47.261 | −18.224 | 1.00 | 27.14 | C |
| ATOM | 3537 | C | PHE | A | 440 | 13.413 | −44.305 | −17.670 | 1.00 | 31.14 | C |
| ATOM | 3538 | O | PHE | A | 440 | 12.252 | −44.431 | −17.932 | 1.00 | 30.17 | O |
| ATOM | 3539 | N | ALA | A | 441 | 14.417 | −43.975 | −17.357 | 1.00 | 31.21 | N |
| ATOM | 3540 | CA | ALA | A | 441 | 14.326 | −42.116 | −17.263 | 1.00 | 32.43 | C |
| ATOM | 3541 | CB | ALA | A | 441 | 13.783 | −41.897 | −18.557 | 1.00 | 34.42 | C |
| ATOM | 3542 | C | ALA | A | 441 | 13.506 | −42.045 | −16.080 | 1.00 | 34.90 | C |
| ATOM | 3543 | O | ALA | A | 441 | 14.050 | −41.520 | −15.063 | 1.00 | 37.01 | O |
| ATOM | 3544 | N | ASP | A | 442 | 12.183 | −42.127 | −16.221 | 1.00 | 36.69 | N |
| ATOM | 3545 | CA | ASP | A | 442 | 11.283 | −41.467 | −15.315 | 1.00 | 33.27 | C |
| ATOM | 3546 | CB | ASP | A | 442 | 10.129 | −40.186 | −16.113 | 1.00 | 35.56 | C |
| ATOM | 3347 | CG | ASP | A | 442 | 9.359 | −41.347 | −16.852 | 1.00 | 37.14 | C |
| ATOM | 3548 | OD1 | ASP | A | 442 | 9.641 | −42.189 | −18.048 | 1.00 | 38.37 | O |
| ATOM | 3549 | OD2 | ASP | A | 442 | 8.484 | −42.164 | −16.213 | 1.00 | 37.82 | O |
| ATOM | 3550 | C | ASP | A | 442 | 10.767 | −42.369 | −14.188 | 1.00 | 32.89 | C |
| ATOM | 3551 | C | ASP | A | 442 | 9.923 | −41.952 | −13.398 | 1.00 | 30.30 | O |
| ATOM | 3552 | N | ASN | A | 443 | 11.297 | −43.581 | −14.101 | 1.00 | 30.07 | N |
| ATOM | 3553 | CA | ASN | A | 443 | 10.808 | −44.567 | −13.159 | 1.00 | 29.57 | C |
| ATOM | 3554 | CB | ASN | A | 443 | 11.158 | −45.989 | −13.641 | 1.00 | 26.20 | C |
| ATOM | 3555 | CG | ASN | A | 443 | 12.664 | −46.233 | −13.704 | 1.00 | 28.08 | C |
| ATOM | 3556 | CD1 | ASN | A | 443 | 13.403 | −45.523 | −14.410 | 1.00 | 25.29 | O |
| ATOM | 3557 | ND2 | ASN | A | 443 | 13.126 | −47.245 | −12.963 | 1.00 | 24.02 | N |
| ATOM | 3558 | C | ASN | A | 443 | 11.260 | −44.328 | −11.718 | 1.00 | 32.98 | C |
| ATOM | 3559 | C | ASN | A | 443 | 10.878 | −48.069 | −10.800 | 1.00 | 34.91 | O |
| ATOM | 3560 | N | GLU | A | 444 | 12.065 | −43.291 | −11.507 | 1.00 | 35.49 | N |
| ATOM | 3561 | CA | GLU | A | 444 | 12.361 | −42.847 | −10.144 | 1.00 | 14.91 | C |
| ATOM | 3562 | CD | GLU | A | 444 | 13.870 | −42.721 | −9.875 | 1.00 | 36.96 | C |
| ATOM | 3563 | CG | GLU | A | 444 | 14.532 | −44.097 | −9.855 | 1.00 | 35.58 | C |
| ATOM | 3564 | CD | GLU | A | 444 | 16.003 | −44.114 | −9.426 | 1.00 | 39.30 | C |
| ATOM | 3565 | OE1 | GLU | A | 444 | 16.590 | −43.090 | −8.993 | 1.00 | 42.13 | O |
| ATOM | 3566 | OE2 | GLU | A | 444 | 16.584 | −45.203 | −9.503 | 1.00 | 39.69 | O |
| ATOM | 3567 | C | GLU | A | 444 | 11.623 | −42.586 | −9.798 | 1.00 | 34.95 | C |
| ATOM | 3568 | O | GLU | A | 444 | 11.833 | −41.024 | −8.745 | 1.00 | 36.50 | O |
| ATOM | 3569 | N | LEU | A | 445 | 10.708 | −41.167 | −10.659 | 1.00 | 36.11 | N |
| ATOM | 3570 | CA | LEU | A | 445 | 9.848 | −40.039 | −10.347 | 1.00 | 38.39 | C |
| ATOM | 3571 | CB | LEU | A | 445 | 9.192 | −40.215 | −8.974 | 1.00 | 36.53 | C |
| ATOM | 3572 | CG | LEU | A | 415 | 7.913 | −41.060 | −8.953 | 1.00 | 38.86 | C |
| ATOM | 3573 | CD1 | LEU | A | 445 | 7.851 | −42.125 | −10.040 | 1.00 | 39.35 | C |
| ATOM | 3574 | CD2 | LEU | A | 445 | 7.749 | −41.682 | −7.583 | 1.00 | 36.78 | C |
| ATOM | 3575 | C | LEU | A | 445 | 10.530 | −38.666 | −10.461 | 1.00 | 39.27 | C |
| ATOM | 3576 | O | LEU | A | 445 | 10.058 | −37.672 | −9.888 | 1.00 | 37.05 | O |
| ATOM | 3577 | N | SER | A | 446 | 11.606 | −38.613 | −11.247 | 1.00 | 39.82 | N |
| ATOM | 3578 | CA | SER | A | 446 | 12.206 | −37.341 | −11.634 | 1.00 | 38.00 | C |
| ATOM | 3579 | CB | SER | A | 446 | 13.297 | −37.559 | −12.668 | 1.00 | 40.03 | C |
| ATOM | 3580 | CG | SER | A | 446 | 14.001 | −36.356 | −12.922 | 1.00 | 39.42 | O |
| ATOM | 3581 | C | SER | A | 446 | 11.190 | −36.289 | −12.122 | 1.00 | 35.49 | C |
| ATOM | 3582 | O | SER | A | 446 | 11.515 | −35.127 | −11.765 | 1.00 | 38.11 | O |
| ATOM | 3583 | N | PRO | A | 447 | 10.178 | −36.682 | −12.917 | 1.00 | 36.32 | N |
| ATOM | 3584 | CA | PRO | A | 447 | 9.180 | −35.658 | −13.281 | 1.00 | 35.17 | C |
| ATOM | 3585 | CB | PRO | A | 447 | 8.176 | −36.442 | −14.132 | 1.00 | 33.91 | C |
| ATOM | 3586 | CG | PRO | A | 447 | 9.049 | −37.453 | −14.815 | 1.00 | 35.97 | C |
| ATOM | 3587 | CD | PRO | A | 447 | 10.044 | −37.883 | −13.771 | 1.00 | 35.20 | C |
| ATOM | 3588 | C | PRO | A | 447 | 8.469 | −35.015 | −12.115 | 1.00 | 36.19 | C |
| ATOM | 3589 | O | PRO | A | 447 | 8.334 | −33.792 | −12.088 | 1.00 | 35.70 | O |
| ATOM | 3590 | N | GLU | A | 448 | 8.012 | −35.830 | −11.163 | 1.00 | 41.44 | N |
| ATOM | 3591 | CA | GLU | A | 448 | 7.148 | −35.341 | −10.092 | 1.00 | 40.30 | C |
| ATOM | 3592 | CB | GLU | A | 448 | 6.463 | −36.491 | −9.339 | 1.00 | 40.88 | C |
| ATOM | 3593 | CG | GLU | A | 448 | 5.367 | −37.251 | −10.098 | 1.00 | 40.59 | C |
| ATOM | 3594 | CD | GLU | A | 448 | 5.870 | −38.158 | −11.221 | 1.00 | 41.65 | C |
| ATOM | 3595 | OE1 | GLU | A | 448 | 7.087 | −58.507 | −11.273 | 1.00 | 41.68 | O |
| ATOM | 3596 | OE2 | GLU | A | 448 | 5.032 | −38.513 | −12.081 | 1.00 | 38.15 | O |
| ATOM | 3597 | C | GLU | A | 448 | 8.011 | −34.513 | −9.158 | 1.00 | 38.67 | C |
| ATOM | 3598 | O | GLU | A | 448 | 7.589 | −33.468 | −8.663 | 1.00 | 35.98 | O |
| ATOM | 3599 | N | CYS | A | 449 | 9.231 | −34.979 | −8.935 | 1.00 | 36.18 | N |
| ATOM | 3600 | CA | CYS | A | 449 | 10.158 | −34.269 | −8.082 | 1.00 | 35.68 | C |
| ATOM | 3601 | CB | CYS | A | 449 | 11.454 | −35.067 | −7.931 | 1.00 | 37.23 | C |
| ATOM | 3602 | SG | CYS | A | 449 | 11.285 | −36.536 | −6.885 | 1.00 | 39.22 | S |
| ATOM | 3603 | C | CYS | A | 449 | 10.451 | −32.876 | −8.656 | 1.00 | 39.15 | C |
| ATOM | 3604 | O | CYS | A | 449 | 10.282 | −31.864 | −7.979 | 1.00 | 39.58 | O |
| ATOM | 3605 | N | LEU | A | 450 | 10.873 | −32.820 | −9.914 | 1.00 | 42.61 | N |
| ATOM | 3606 | CA | LEU | A | 450 | 11.250 | −31.555 | −10.502 | 1.00 | 42.01 | C |
| ATOM | 3607 | CB | LEU | A | 450 | 12.039 | −31.769 | −11.784 | 1.00 | 40.39 | C |
| ATOM | 3608 | CG | LEU | A | 450 | 13.445 | −32.394 | −11.668 | 1.00 | 46.06 | C |
| ATOM | 3609 | CD1 | LEU | A | 450 | 14.103 | −32.533 | −13.045 | 1.00 | 40.62 | C |
| ATOM | 3610 | CD2 | LEU | A | 450 | 14.372 | −31.657 | −10.693 | 1.00 | 41.29 | C |
| ATOM | 3611 | C | LEU | A | 450 | 10.043 | −30.617 | −10.698 | 1.00 | 44.72 | C |
| ATOM | 3612 | O | LEU | A | 450 | 10.182 | −29.395 | −10.561 | 1.00 | 44.85 | O |
| ATOM | 3613 | N | ASP | A | 451 | 8.873 | −31.183 | −10.992 | 1.00 | 43.98 | N |
| ATOM | 3614 | CA | ASP | A | 451 | 7.650 | −30.392 | −11.128 | 1.00 | 44.28 | C |
| ATOM | 3615 | CB | ASP | A | 451 | 6.427 | −31.287 | −11.392 | 1.00 | 45.24 | C |
| ATOM | 3616 | CG | ASP | A | 451 | 6.323 | −31.744 | −12.819 | 1.00 | 47.47 | C |
| ATOM | 3617 | OD1 | ASP | A | 451 | 7.263 | −31.489 | −13.606 | 1.00 | 47.02 | O |
| ATOM | 3618 | OD2 | ASP | A | 451 | 5.289 | −32.374 | −13.147 | 1.00 | 50.02 | O |
| ATOM | 3619 | C | ASP | A | 451 | 7.359 | −29.514 | −9.861 | 1.00 | 43.71 | C |
| ATOM | 3620 | O | ASP | A | 451 | 6.881 | −28.485 | −9.916 | 1.00 | 44.74 | O |
| ATOM | 3621 | N | GLY | A | 452 | 7.588 | −30.254 | −8.719 | 1.00 | 43.07 | N |
| ATOM | 3622 | CA | GLY | A | 452 | 7.249 | −29.666 | −7.432 | 1.00 | 43.54 | C |
| ATOM | 3623 | C | GLY | A | 452 | 8.332 | −28.722 | −6.946 | 1.00 | 43.28 | C |
| ATOM | 3624 | O | GLY | A | 452 | 8.221 | −28.145 | −5.885 | 1.00 | 47.72 | O |
| ATOM | 3625 | N | ALA | A | 453 | 9.376 | −28.573 | −7.745 | 1.00 | 43.67 | N |
| ATOM | 3626 | CA | ALA | A | 453 | 10.517 | −27.732 | −7.421 | 1.00 | 46.71 | C |
| ATOM | 3627 | CB | ALA | A | 453 | 11.823 | −28.512 | −7.591 | 1.00 | 37.90 | C |
| ATOM | 3628 | C | ALA | A | 453 | 10.522 | −26.518 | −8.335 | 1.00 | 47.26 | C |
| ATOM | 3629 | O | ALA | A | 453 | 10.948 | −25.437 | −7.936 | 1.00 | 48.26 | O |
| ATOM | 3630 | N | GLN | A | 454 | 10.039 | −26.700 | −9.559 | 1.00 | 49.01 | N |
| ATOM | 3631 | CA | GLN | A | 454 | 10.246 | −25.699 | −10.591 | 1.00 | 55.86 | C |
| ATOM | 3632 | CB | GLN | A | 454 | 9.746 | −26.186 | −11.956 | 1.00 | 56.88 | C |
| ATOM | 3633 | CG | GLN | A | 454 | 8.240 | −26.196 | −12.147 | 1.00 | 53.74 | C |
| ATOM | 3634 | CD | GLN | A | 454 | 7.824 | −26.564 | −13.536 | 1.00 | 53.69 | C |
| ATOM | 3635 | OE1 | GLN | A | 454 | 8.653 | −27.068 | −14.356 | 1.00 | 50.68 | O |
| ATOM | 3636 | NE2 | GLN | A | 454 | 6.520 | −26.531 | −13.798 | 1.00 | 58.30 | N |
| ATOM | 3637 | C | GLN | A | 454 | 9.730 | −24.293 | −10.244 | 1.00 | 56.30 | C |
| ATOM | 3638 | O | GLN | A | 454 | 10.262 | −23.308 | −10.758 | 1.00 | 56.85 | O |
| ATOM | 3639 | N | HIS | A | 455 | 8.728 | −24.201 | −9.366 | 1.00 | 56.87 | N |
| ATOM | 3640 | CA | HIS | A | 455 | 8.212 | −22.398 | −8.911 | 1.00 | 58.44 | C |
| ATOM | 3641 | CB | HIS | A | 455 | 7.139 | −23.081 | −7.843 | 1.00 | 55.12 | C |
| ATOM | 3642 | CG | HIS | A | 455 | 7.673 | −23.511 | −6.551 | 1.00 | 56.09 | C |
| ATOM | 3643 | ND1 | HIS | A | 455 | 8.046 | −22.793 | −5.507 | 1.00 | 56.78 | N |
| ATOM | 3644 | CE1 | HIS | A | 455 | 8.481 | −23.531 | −4.504 | 1.00 | 53.88 | C |
| ATOM | 3645 | NE2 | HIS | A | 455 | 8.411 | −24.800 | −4.866 | 1.00 | 52.42 | N |
| ATOM | 3646 | CD2 | HIS | A | 455 | 7.921 | −24.377 | −6.142 | 1.00 | 55.29 | C |
| ATOM | 3647 | C | HIS | A | 455 | 9.315 | −21.977 | −8.364 | 1.00 | 59.08 | C |
| ATOM | 3648 | O | HIS | A | 455 | 9.113 | −20.768 | −8.258 | 1.00 | 62.19 | O |
| ATOM | 3649 | N | PHE | A | 456 | 10.464 | −22.543 | −7.995 | 1.00 | 56.78 | N |
| ATOM | 3650 | CA | PHE | A | 456 | 11.603 | −21.723 | −7.583 | 1.00 | 50.65 | C |
| ATOM | 3651 | CB | PHE | A | 456 | 12.082 | −22.060 | −6.162 | 1.00 | 50.01 | C |
| ATOM | 3652 | CG | PHE | A | 456 | 12.617 | −23.453 | −5.983 | 1.00 | 49.62 | C |
| ATOM | 3653 | CD1 | PHE | A | 456 | 13.873 | −23.312 | −6.472 | 1.00 | 50.07 | C |
| ATOM | 3654 | CE1 | PHE | A | 456 | 14.377 | −25.092 | −6.278 | 1.00 | 48.90 | C |
| ATOM | 3655 | CZ | PHE | A | 456 | 13.639 | −26.024 | −5.555 | 1.00 | 49.75 | C |
| ATOM | 3656 | CE2 | PHE | A | 456 | 12.396 | −25.574 | −5.045 | 1.00 | 50.47 | C |
| ATOM | 3657 | CD2 | PHE | A | 456 | 11.895 | −24.394 | −5.252 | 1.00 | 50.17 | C |
| ATOM | 3658 | C | PHE | A | 456 | 12.748 | −21.563 | −8.600 | 1.00 | 51.88 | C |
| ATOM | 3659 | O | PHE | A | 456 | 13.886 | −21.314 | −8.266 | 1.00 | 52.26 | O |
| ATOM | 3600 | N | LEU | A | 457 | 12.429 | −21.991 | −9.846 | 1.00 | 52.58 | N |
| ATOM | 3661 | CA | LEU | A | 457 | 13.314 | −21.742 | −10.967 | 1.00 | 53.51 | C |
| ATOM | 3662 | CB | LEU | A | 457 | 13.223 | −22.894 | −11.972 | 1.00 | 51.36 | C |
| ATOM | 3663 | CG | LEU | A | 457 | 14.201 | −22.951 | −13.161 | 1.00 | 51.83 | C |
| ATOM | 3664 | CD1 | LEU | A | 457 | 15.598 | −23.426 | −12.768 | 1.00 | 47.88 | C |
| ATOM | 3665 | CD2 | LEU | A | 457 | 13.643 | −23.316 | −14.284 | 1.00 | 49.11 | C |
| ATOM | 3666 | C | LEU | A | 457 | 12.896 | −20.421 | −11.610 | 1.00 | 58.86 | C |
| ATOM | 3667 | O | LEU | A | 457 | 11.698 | −20.179 | −11.845 | 1.00 | 61.53 | O |
| ATOM | 3668 | N | LYS | A | 458 | 13.870 | −19.354 | −11.886 | 1.00 | 61.04 | N |
| ATOM | 3669 | CA | LYS | A | 458 | 13.566 | −18.254 | −12.522 | 1.00 | 65.44 | C |
| ATOM | 3670 | CB | LYS | A | 458 | 14.817 | −17.272 | −12.629 | 1.00 | 65.97 | C |
| ATOM | 3671 | CG | LYS | A | 458 | 15.367 | −16.328 | −11.275 | 1.00 | 69.42 | C |
| ATOM | 3672 | CD | LYS | A | 458 | 16.342 | −15.767 | −11.364 | 1.00 | 71.98 | C |
| ATOM | 3673 | CE | LYS | A | 458 | 17.662 | −16.193 | −11.981 | 1.00 | 69.50 | C |
| ATOM | 3674 | NZ | LYS | A | 458 | 18.649 | −15.077 | −11.934 | 1.00 | 76.12 | N |
| ATOM | 3675 | C | LYS | A | 458 | 12.921 | −18.454 | −13.888 | 1.00 | 64.10 | C |
| ATOM | 3676 | O | LYS | A | 458 | 13.089 | −19.513 | −14.495 | 1.00 | 62.83 | O |
| ATOM | 3677 | N | ASP | A | 459 | 12.185 | −17.445 | −14.360 | 1.00 | 66.20 | N |
| ATOM | 3678 | CA | ASP | A | 459 | 11.379 | −17.566 | −15.594 | 1.00 | 62.13 | C |
| ATOM | 3679 | CB | ASP | A | 459 | 10.603 | −16.273 | −15.883 | 1.00 | 64.79 | C |
| ATOM | 3680 | CG | ASP | A | 459 | 9.410 | −16.082 | −14.945 | 1.00 | 68.11 | C |
| ATOM | 3681 | CD1 | ASP | A | 459 | 9.096 | −17.005 | −14.169 | 1.00 | 71.41 | O |
| ATOM | 3682 | CD2 | ASP | A | 459 | 8.775 | −15.009 | −14.976 | 1.00 | 68.15 | O |
| ATOM | 3683 | C | ASP | A | 459 | 12.173 | −18.025 | −16.818 | 1.00 | 57.16 | C |
| ATOM | 3684 | O | ASP | A | 459 | 11.636 | −18.715 | −17.683 | 1.00 | 54.66 | O |
| ATOM | 3685 | N | ASP | A | 460 | 13.444 | −17.630 | −16.868 | 1.00 | 52.18 | N |
| ATOM | 3686 | CA | ASP | A | 460 | 14.372 | −18.005 | −17.926 | 1.00 | 51.95 | C |
| ATOM | 3687 | CB | ASP | A | 460 | 15.235 | −16.785 | −18.322 | 1.00 | 57.42 | C |
| ATOM | 3688 | CG | ASP | A | 460 | 16.060 | −16.228 | −17.139 | 1.00 | 59.48 | C |
| ATOM | 3689 | CD1 | ASP | A | 460 | 15.735 | −15.123 | −16.666 | 1.00 | 67.30 | O |
| ATOM | 3690 | CD2 | ASP | A | 460 | 17.005 | −16.896 | −16.652 | 1.00 | 57.79 | O |
| ATOM | 3691 | C | ASP | A | 460 | 15.304 | −19.118 | −17.443 | 1.00 | 49.25 | C |
| ATOM | 3692 | O | ASP | A | 460 | 16.377 | −19.325 | −18.020 | 1.00 | 45.85 | O |
| ATOM | 3693 | N | GLY | A | 461 | 14.915 | −19.802 | −16.369 | 1.00 | 49.05 | N |
| ATOM | 3694 | CA | GLY | A | 161 | 15.789 | −20.783 | −15.707 | 1.00 | 52.89 | C |
| ATOM | 3695 | C | GLY | A | 461 | 16.067 | −22.022 | −16.539 | 1.00 | 48.92 | C |
| ATOM | 3696 | O | GLY | A | 461 | 15.282 | −22.383 | −17.417 | 1.00 | 49.28 | O |
| ATOM | 3697 | N | VAL | A | 462 | 17.206 | −22.643 | −16.274 | 1.00 | 46.61 | N |
| ATOM | 3698 | CA | VAL | A | 462 | 17.604 | −23.871 | −16.941 | 1.00 | 45.73 | C |
| ATOM | 3699 | CB | VAL | A | 462 | 19.042 | −23.794 | −17.490 | 1.00 | 46.56 | C |
| ATOM | 3700 | CG1 | VAL | A | 462 | 19.473 | −25.127 | −18.077 | 1.00 | 44.99 | C |
| ATOM | 3701 | CG2 | VAL | A | 462 | 19.155 | −22.718 | −18.549 | 1.00 | 48.32 | C |
| ATOM | 3702 | C | VAL | A | 462 | 17.526 | −25.037 | −15.954 | 1.00 | 45.90 | C |
| ATOM | 3703 | O | VAL | A | 462 | 17.967 | −24.928 | −14.801 | 1.00 | 43.87 | O |
| ATOM | 3704 | N | SER | A | 463 | 16.950 | −26.148 | −16.413 | 1.00 | 42.18 | N |
| ATOM | 3705 | CA | SER | A | 463 | 17.045 | −27.401 | −15.692 | 1.00 | 38.29 | C |
| ATOM | 3706 | CB | SER | A | 463 | 15.686 | −28.111 | −15.669 | 1.00 | 41.25 | C |
| ATOM | 3707 | OG | SER | A | 463 | 15.823 | −29.436 | −15.193 | 1.00 | 42.50 | O |
| ATOM | 3708 | C | SER | A | 463 | 18.085 | −28.275 | −16.371 | 1.00 | 35.76 | C |
| ATOM | 3709 | O | SER | A | 463 | 18.059 | −28.458 | −17.588 | 1.00 | 34.40 | O |
| ATOM | 3710 | N | ILE | A | 464 | 19.007 | −28.792 | −15.583 | 1.00 | 33.38 | N |
| ATOM | 3711 | CA | ILE | A | 464 | 19.873 | −29.840 | −16.038 | 1.00 | 34.53 | C |
| ATOM | 3712 | CB | ILE | A | 464 | 21.351 | −29.420 | −15.922 | 1.00 | 33.64 | C |
| ATOM | 3713 | CG1 | ILE | A | 464 | 21.659 | −28.311 | −16.935 | 1.00 | 35.95 | C |
| ATOM | 3714 | CD1 | ILE | A | 464 | 22.934 | −27.532 | −16.642 | 1.00 | 36.35 | C |
| ATOM | 3715 | CG2 | ILE | A | 464 | 22.258 | −30.618 | −16.147 | 1.00 | 32.17 | C |
| ATOM | 3716 | C | ILE | A | 464 | 19.573 | −31.108 | −15.213 | 1.00 | 36.22 | C |
| ATOM | 3717 | O | ILE | A | 464 | 19.893 | −31.181 | −14.005 | 1.00 | 37.04 | O |
| ATOM | 3718 | N | PRO | A | 465 | 18.985 | −32.134 | −15.857 | 1.00 | 37.82 | N |
| ATOM | 3719 | CA | PRO | A | 465 | 18.728 | −32.342 | −17.292 | 1.00 | 36.47 | C |
| ATOM | 3720 | CB | PRO | A | 465 | 18.326 | −33.821 | −17.381 | 1.00 | 39.49 | C |
| ATOM | 3721 | CG | PRO | A | 465 | 18.280 | −34.345 | −15.980 | 1.00 | 38.33 | C |
| ATOM | 3722 | CD | PRO | A | 465 | 18.382 | −33.197 | −15.032 | 1.00 | 38.98 | C |
| ATOM | 3723 | C | PRO | A | 465 | 17.589 | −31.521 | −17.816 | 1.00 | 34.71 | C |
| ATOM | 3724 | O | PRO | A | 465 | 16.688 | −31.143 | −17.036 | 1.00 | 31.60 | O |
| ATOM | 3725 | N | GLY | A | 466 | 17.626 | −31.246 | −19.121 | 1.00 | 32.54 | N |
| ATOM | 3726 | CA | GLY | A | 466 | 16.502 | −30.565 | −19.784 | 1.00 | 35.93 | C |
| ATOM | 3727 | C | GLY | A | 466 | 15.452 | −31.537 | −20.287 | 1.00 | 37.70 | C |
| ATOM | 3728 | O | GLY | A | 466 | 14.269 | −31.208 | −20.402 | 1.00 | 40.35 | O |
| ATOM | 3729 | N | GLU | A | 467 | 15.874 | −32.768 | −20.545 | 1.00 | 39.25 | N |
| ATOM | 3730 | CA | GLU | A | 467 | 15.023 | −33.707 | −21.237 | 1.00 | 42.33 | C |
| ATOM | 3731 | CB | GLU | A | 467 | 15.091 | −33.414 | −22.728 | 1.00 | 47.99 | C |
| ATOM | 3732 | CG | GLU | A | 467 | 13.804 | −33.658 | −23.475 | 1.00 | 52.47 | C |
| ATOM | 3733 | CD | GLU | A | 467 | 14.025 | −33.685 | −24.975 | 1.00 | 56.29 | C |
| ATOM | 3734 | OE1 | GLU | A | 467 | 14.899 | −34.439 | −25.447 | 1.00 | 55.85 | O |
| ATOM | 3735 | OE2 | GLU | A | 467 | 13.319 | −32.946 | −25.682 | 1.00 | 61.10 | O |
| ATOM | 3736 | C | GLU | A | 467 | 15.472 | −35.125 | −21.004 | 1.00 | 40.60 | C |
| ATOM | 3737 | O | GLU | A | 467 | 16.671 | −35.390 | −20.859 | 1.00 | 42.00 | O |
| ATOM | 3738 | N | TYR | A | 468 | 14.508 | −36.037 | −20.964 | 1.00 | 36.50 | N |
| ATOM | 3739 | CA | TYR | A | 468 | 14.819 | −37.472 | −21.004 | 1.00 | 35.84 | |
| ATOM | 3740 | CB | TYR | A | 468 | 15.138 | −38.356 | −19.604 | 1.00 | 35.20 | C |
| ATOM | 3741 | CG | TYR | A | 468 | 14.351 | −37.502 | −18.433 | 1.00 | 33.67 | C |
| ATOM | 3742 | CD1 | TYR | A | 468 | 13.110 | −38.347 | −18.068 | 1.00 | 36.91 | C |
| ATOM | 3743 | CE1 | TYR | A | 468 | 12.385 | −37.541 | −16.999 | 1.00 | 34.65 | C |
| ATOM | 3744 | CZ | TYR | A | 468 | 32.909 | −36.487 | −16.256 | 1.00 | 38.00 | C |
| ATOM | 3745 | OH | TYR | A | 468 | 12.200 | −35.973 | −15.179 | 1.00 | 35.86 | O |
| ATOM | 3746 | CE2 | TYR | A | 468 | 14.152 | −35.946 | −16.590 | 1.00 | 34.55 | C |
| ATOM | 3747 | CD2 | TYR | A | 468 | 14.853 | −36.460 | −17.671 | 1.00 | 34.10 | C |
| ATOM | 3748 | C | TYR | A | 468 | 13.762 | −38.292 | −21.749 | 1.00 | 35.53 | C |
| ATOM | 3749 | O | TYR | A | 468 | 12.566 | −37.922 | −21.799 | 1.00 | 34.36 | O |
| ATOM | 3750 | N | THR | A | 469 | 14.211 | −39.401 | −22.332 | 1.00 | 34.97 | N |
| ATOM | 3751 | CA | THR | A | 469 | 13.345 | −40.259 | −23.140 | 1.00 | 34.32 | C |
| ATOM | 3752 | CB | THR | A | 469 | 13.580 | −39.369 | −24.638 | 1.00 | 35.21 | C |
| ATOM | 3753 | OG1 | THR | A | 469 | 13.176 | −38.625 | −24.914 | 1.00 | 36.49 | O |
| ATOM | 3754 | CG2 | THR | A | 469 | 12.807 | −40.941 | −25.563 | 1.00 | 33.21 | C |
| ATOM | 3755 | C | THR | A | 469 | 13.600 | −41.740 | −22.827 | 1.00 | 33.51 | C |
| ATOM | 3756 | O | THR | A | 469 | 14.744 | −42.156 | −22.775 | 1.00 | 34.44 | O |
| ATOM | 3757 | N | SER | A | 470 | 12.539 | −42.527 | −22.639 | 1.00 | 31.27 | N |
| ATOM | 3758 | CA | SER | A | 470 | 12.689 | −43.967 | −22.421 | 1.00 | 28.62 | C |
| ATOM | 3759 | CB | SER | A | 470 | 11.708 | −44.491 | −21.370 | 1.00 | 30.06 | C |
| ATOM | 3760 | OG | SER | A | 470 | 10.368 | −44.250 | −21.745 | 1.00 | 33.04 | O |
| ATOM | 3761 | C | SER | A | 470 | 12.561 | −44.744 | −23.721 | 1.00 | 26.98 | C |
| ATOM | 3762 | O | SER | A | 470 | 12.077 | −44.228 | −24.732 | 1.00 | 27.81 | O |
| ATOM | 3763 | N | PHE | A | 471 | 13.048 | −45.972 | −23.718 | 1.00 | 26.36 | N |
| ATOM | 3764 | CA | PHE | A | 471 | 13.015 | −46.799 | −24.909 | 1.00 | 27.71 | C |
| ATOM | 3765 | CB | PHE | A | 471 | 14.363 | −46.323 | −25.594 | 1.00 | 28.62 | C |
| ATOM | 3766 | CG | PHE | A | 471 | 14.735 | −45.550 | −26.261 | 1.00 | 30.95 | C |
| ATOM | 3767 | CD1 | PHE | A | 471 | 15.306 | −44.510 | −25.528 | 1.00 | 31.54 | C |
| ATOM | 3768 | CE1 | PHE | A | 471 | 15.684 | −43.335 | −26.143 | 1.00 | 34.67 | C |
| ATOM | 3769 | CZ | PHE | A | 471 | 15.521 | −43.195 | −27.513 | 1.00 | 37.25 | C |
| ATOM | 3770 | CE2 | PHE | A | 471 | 14.964 | −44.238 | −28.262 | 1.00 | 37.40 | C |
| ATOM | 3771 | CD2 | PHE | A | 471 | 14.585 | −45.414 | −27.634 | 1.00 | 32.17 | C |
| ATOM | 3772 | C | PHE | A | 471 | 12.705 | −48.221 | −24.530 | 1.00 | 27.45 | C |
| ATOM | 3773 | O | PHE | A | 471 | 13.040 | −48.554 | −23.433 | 1.00 | 29.36 | O |
| ATOM | 3774 | N | LEU | A | 472 | 12.110 | −48.359 | −25.464 | 1.00 | 26.18 | N |
| ATOM | 3775 | CA | LEU | A | 472 | 11.812 | −50.361 | −25.230 | 1.00 | 25.44 | C |
| ATOM | 3776 | CB | LEU | A | 472 | 10.308 | −50.555 | −24.973 | 1.00 | 26.32 | C |
| ATOM | 3777 | CG | LEU | A | 472 | 9.747 | −50.074 | −23.651 | 1.00 | 26.42 | C |
| ATOM | 3778 | CD1 | LEU | A | 472 | 8.214 | −50.032 | −23.748 | 1.00 | 28.93 | C |
| ATOM | 3779 | CD2 | LEU | A | 472 | 10.221 | −51.009 | −22.553 | 1.00 | 22.56 | C |
| ATOM | 3780 | C | LEU | A | 472 | 12.209 | −51.122 | −26.453 | 1.00 | 24.46 | C |
| ATOM | 3781 | O | LEU | A | 472 | 12.144 | −50.577 | −27.544 | 1.00 | 23.87 | O |
| ATOM | 3782 | N | ALA | A | 473 | 12.632 | −52.366 | −26.253 | 1.00 | 25.21 | N |
| ATOM | 3783 | CA | ALA | A | 473 | 12.862 | −53.331 | −27.323 | 1.00 | 27.08 | C |
| ATOM | 3784 | CB | ALA | A | 473 | 14.358 | −53.419 | −27.624 | 1.00 | 27.57 | C |
| ATOM | 3785 | C | ALA | A | 473 | 12.348 | −54.690 | −26.825 | 1.00 | 28.80 | C |
| ATOM | 3786 | O | ALA | A | 473 | 12.564 | −55.044 | −25.657 | 1.00 | 28.40 | O |
| ATOM | 3787 | N | PRO | A | 474 | 11.671 | −55.465 | −27.686 | 1.00 | 30.26 | N |
| ATOM | 3788 | CA | PRO | A | 474 | 11.257 | −56.792 | −27.208 | 1.00 | 33.51 | C |
| ATOM | 3789 | CB | PRO | A | 474 | 10.263 | −57.257 | −28.276 | 1.00 | 29.99 | C |
| ATOM | 3790 | CG | PRO | A | 474 | 10.688 | −56.549 | −29.497 | 1.00 | 29.65 | C |
| ATOM | 3791 | CD | PRO | A | 474 | 11.164 | −55.194 | −29.032 | 1.00 | 31.91 | C |
| ATOM | 3792 | C | PRO | A | 474 | 12.452 | −57.742 | −27.162 | 1.00 | 33.68 | C |
| ATOM | 3793 | O | PRO | A | 474 | 13.356 | −57.597 | −27.965 | 1.00 | 36.48 | O |
| ATOM | 3794 | N | ILE | A | 475 | 12.439 | −58.697 | −26.243 | 1.00 | 32.95 | N |
| ATOM | 3795 | CA | ILE | A | 475 | 13.514 | −59.644 | −26.135 | 1.00 | 35.44 | C |
| ATOM | 3796 | CB | ILE | A | 475 | 14.433 | −59.368 | −24.929 | 1.00 | 35.15 | C |
| ATOM | 3797 | CG1 | ILE | A | 475 | 13.657 | −59.463 | −23.640 | 1.00 | 34.40 | C |
| ATOM | 3798 | CD1 | ILE | A | 475 | 14.421 | −60.197 | −22.568 | 1.00 | 36.57 | C |
| ATOM | 3799 | CG2 | ILE | A | 475 | 15.083 | −58.010 | −25.048 | 1.00 | 36.57 | C |
| ATOM | 3800 | C | ILE | A | 475 | 13.025 | −61.072 | −26.022 | 1.00 | 37.97 | C |
| ATOM | 3801 | O | ILE | A | 475 | 11.935 | −61.348 | −25.505 | 1.00 | 37.69 | O |
| ATOM | 3802 | N | SER | A | 476 | 13.852 | −61.975 | −26.540 | 1.00 | 38.71 | N |
| ATOM | 3803 | CA | SER | A | 476 | 13.732 | −63.377 | −26.212 | 1.00 | 41.02 | C |
| ATOM | 3804 | CB | SER | A | 476 | 14.034 | −64.228 | −27.438 | 1.00 | 42.98 | C |
| ATOM | 3805 | OG | SER | A | 476 | 13.439 | −65.491 | −27.270 | 1.00 | 49.06 | O |
| ATOM | 3806 | C | SER | A | 476 | 14.739 | −63.719 | −25.140 | 1.00 | 38.30 | C |
| ATOM | 3807 | O | SER | A | 476 | 15.896 | −63.351 | −25.248 | 1.00 | 40.90 | O |
| ATOM | 3808 | N | SER | A | 477 | 14.310 | −64.415 | −24.101 | 1.00 | 38.25 | N |
| ATOM | 3809 | CA | SER | A | 477 | 15.261 | −65.066 | −23.212 | 1.00 | 37.24 | C |
| ATOM | 3810 | CB | SER | A | 477 | 16.121 | −64.106 | −22.400 | 1.00 | 34.49 | C |
| ATOM | 3811 | OG | SER | A | 477 | 16.596 | −64.777 | −21.225 | 1.00 | 31.70 | O |
| ATOM | 3812 | C | SER | A | 477 | 14.517 | −65.947 | −22.274 | 1.00 | 37.97 | C |
| ATOM | 3813 | O | SER | A | 477 | 13.838 | −65.490 | −21.365 | 1.00 | 38.64 | O |
| ATOM | 3814 | N | SER | A | 478 | 14.721 | −67.231 | −22.457 | 1.00 | 41.24 | N |
| ATOM | 3815 | CA | SER | A | 478 | 14.101 | −68.197 | −21.594 | 1.00 | 41.48 | C |
| ATOM | 3816 | CG | SER | A | 478 | 14.022 | −69.524 | −22.330 | 1.00 | 39.86 | C |
| ATOM | 3817 | OG | SER | A | 478 | 13.681 | −69.260 | −23.720 | 1.00 | 37.54 | O |
| ATOM | 3818 | C | SER | A | 478 | 14.843 | −68.236 | −20.267 | 1.00 | 43.44 | C |
| ATOM | 3819 | O | SER | A | 478 | 14.250 | −68.484 | −19.218 | 1.00 | 46.81 | O |
| ATOM | 3820 | N | LYS | A | 479 | 16.132 | −67.918 | −20.295 | 1.00 | 49.48 | C |
| ATOM | 3821 | CA | LYS | A | 479 | 16.901 | −67.829 | −19.042 | 1.00 | 49.54 | C |
| ATOM | 3822 | CB | LYS | A | 479 | 18.383 | −67.507 | −19.333 | 1.00 | 49.96 | C |
| ATOM | 3823 | CG | LYS | A | 479 | 19.185 | −66.872 | −18.194 | 1.00 | 54.04 | C |
| ATOM | 3824 | CD | LYS | A | 479 | 19.444 | −67.824 | −17.033 | 1.00 | 58.10 | C |
| ATOM | 3825 | CE | LYS | A | 479 | 20.404 | −67.239 | −16.005 | 1.00 | 58.81 | C |
| ATOM | 3826 | NZ | LYS | A | 479 | 20.268 | −67.926 | −14.680 | 1.00 | 59.05 | N |
| ATOM | 3827 | C | LYS | A | 479 | 16.249 | −66.818 | −18.080 | 1.00 | 45.25 | C |
| ATOM | 3828 | O | LYS | A | 479 | 15.952 | −67.148 | −16.938 | 1.00 | 47.22 | O |
| ATOM | 3829 | N | LEU | A | 480 | 16.016 | −65.599 | −18.568 | 1.00 | 41.05 | N |
| ATOM | 3830 | CA | LEU | A | 480 | 15.438 | −64.512 | −17.769 | 1.00 | 37.55 | C |
| ATOM | 3831 | CB | LEU | A | 480 | 15.621 | −63.176 | −18.488 | 1.00 | 39.10 | C |
| ATOM | 3832 | CG | LEU | A | 480 | 17.062 | −62.638 | −18.446 | 1.00 | 40.63 | C |
| ATOM | 3833 | CD1 | LEU | A | 480 | 17.241 | −61.466 | −19.392 | 1.00 | 35.94 | C |
| ATOM | 3834 | CD2 | LEU | A | 480 | 17.449 | −62.271 | −17.011 | 1.00 | 35.26 | C |
| ATOM | 3835 | C | LEU | A | 480 | 13.967 | −64.730 | −17.423 | 1.00 | 37.13 | C |
| ATOM | 3836 | O | LEU | A | 480 | 13.532 | −64.486 | −16.277 | 1.00 | 35.35 | O |
| ATOM | 3837 | N | TYR | A | 481 | 13.204 | −65.214 | −18.399 | 1.00 | 36.73 | N |
| ATOM | 3838 | CA | TYR | A | 481 | 11.852 | −65.667 | −18.105 | 1.00 | 39.08 | C |
| ATOM | 3839 | CB | TYR | A | 481 | 11.240 | −66.424 | −19.291 | 1.00 | 39.31 | O |
| ATOM | 3840 | CG | TYR | A | 481 | 9.851 | −66.976 | −18.970 | 1.00 | 43.01 | C |
| ATOM | 3841 | CD1 | TYR | A | 481 | 8.729 | −66.145 | −19.019 | 1.00 | 39.80 | C |
| ATOM | 3842 | CE1 | TYR | A | 481 | 7.468 | −66.624 | −18.714 | 1.00 | 42.47 | C |
| ATOM | 3843 | CZ | TYR | A | 481 | 7.296 | −67.959 | −18.367 | 1.00 | 44.78 | C |
| ATOM | 3844 | OH | TYR | A | 481 | 6.020 | −68.404 | −18.070 | 1.00 | 47.31 | O |
| ATOM | 3845 | CE2 | TYR | A | 481 | 8.394 | −68.821 | −18.300 | 1.00 | 44.74 | C |
| ATOM | 3846 | CD2 | TYR | A | 481 | 9.663 | −68.329 | −18.597 | 1.00 | 41.69 | C |
| ATOM | 3847 | C | TYR | A | 481 | 11.890 | −66.551 | −16.870 | 1.00 | 37.00 | C |
| ATOM | 3848 | O | TYR | A | 481 | 11.096 | −66.399 | −15.942 | 1.00 | 39.68 | O |
| ATOM | 3849 | N | ASN | A | 482 | 12.840 | −67.469 | −16.850 | 1.00 | 40.44 | N |
| ATOM | 3850 | CA | ASN | A | 482 | 12.911 | −68.446 | −15.776 | 1.00 | 42.63 | C |
| ATOM | 3851 | CB | ASN | A | 482 | 13.772 | −69.616 | −16.217 | 1.00 | 49.98 | C |
| ATOM | 3852 | CG | ASN | A | 482 | 13.116 | −70.941 | −15.947 | 1.00 | 53.15 | C |
| ATOM | 3853 | OD1 | ASN | A | 482 | 12.250 | −71.055 | −15.082 | 1.00 | 60.93 | O |
| ATOM | 3854 | ND2 | ASN | A | 482 | 13.525 | −71.956 | −16.687 | 1.00 | 54.03 | N |
| ATOM | 3855 | C | ASN | A | 482 | 13.392 | −67.868 | −14.461 | 1.00 | 41.27 | C |
| ATOM | 3856 | O | ASN | A | 482 | 12.958 | −68.279 | −13.384 | 1.00 | 45.15 | O |
| ATOM | 3857 | N | GLU | A | 483 | 14.287 | −66.897 | −14.557 | 1.00 | 40.44 | N |
| ATOM | 3858 | CA | GLU | A | 483 | 14.707 | −66.132 | −13.408 | 1.00 | 40.71 | C |
| ATOM | 3859 | CB | GLU | A | 483 | 15.847 | −65.192 | −13.829 | 1.00 | 48.52 | C |
| ATOM | 3860 | CG | GLU | A | 483 | 17.218 | −65.855 | −13.906 | 1.00 | 54.49 | C |
| ATOM | 3861 | CD | GLU | A | 483 | 17.896 | −65.875 | −12.545 | 1.00 | 64.45 | C |
| ATOM | 3862 | CE1 | GLU | A | 483 | 18.350 | −64.795 | −12.088 | 1.00 | 74.66 | O |
| ATOM | 3863 | OE2 | GLU | A | 483 | 17.959 | −66.953 | −11.915 | 1.00 | 61.65 | O |
| ATOM | 3864 | C | GLU | A | 483 | 13.548 | −65.334 | −12.804 | 1.00 | 33.63 | C |
| ATOM | 3865 | O | GLU | A | 483 | 13.418 | −65.231 | −11.598 | 1.00 | 30.55 | O |
| ATOM | 3866 | N | VAL | A | 484 | 12.709 | −64.736 | −13.638 | 1.00 | 33.60 | N |
| ATOM | 3867 | CA | VAL | A | 484 | 11.549 | −63.993 | −13.109 | 1.00 | 32.59 | C |
| ATOM | 3868 | CB | VAL | A | 484 | 10.782 | −63.243 | −14.218 | 1.00 | 33.26 | C |
| ATOM | 3869 | CG1 | VAL | A | 484 | 9.500 | −62.599 | −13.670 | 1.00 | 34.88 | C |
| ATOM | 3870 | CG2 | VAL | A | 484 | 11.680 | −62.230 | −14.900 | 1.00 | 31.77 | C |
| ATOM | 3871 | C | VAL | A | 484 | 10.606 | −64.959 | −12.408 | 1.00 | 33.54 | C |
| ATOM | 3872 | O | VAL | A | 484 | 10.238 | −64.754 | −11.249 | 1.00 | 34.98 | O |
| ATOM | 3873 | N | ARG | A | 485 | 10.238 | −66.022 | −13.119 | 1.00 | 37.13 | N |
| ATOM | 3874 | CA | ARG | A | 485 | 9.315 | −67.031 | −12.609 | 1.00 | 43.14 | C |
| ATOM | 3875 | CB | ARG | A | 485 | 9.250 | −68.215 | −13.578 | 1.00 | 47.02 | C |
| ATOM | 3876 | CG | ARG | A | 485 | 7.851 | −68.762 | −13.771 | 1.00 | 51.39 | C |
| ATOM | 3877 | CD | ARG | A | 485 | 7.803 | −69.363 | −14.807 | 1.00 | 53.67 | C |
| ATOM | 3878 | NE | ARG | A | 485 | 8.755 | −70.903 | −14.442 | 1.00 | 64.44 | N |
| ATOM | 3879 | CZ | ARG | A | 485 | 8.574 | −72.203 | −14.636 | 1.00 | 59.60 | C |
| ATOM | 3880 | NH1 | ARG | A | 485 | 7.447 | −72.544 | −15.197 | 1.00 | 52.57 | N |
| ATOM | 3881 | NH2 | ARG | A | 485 | 9.527 | −73.051 | −14.247 | 1.00 | 53.85 | N |
| ATOM | 3882 | C | ARG | A | 485 | 9.776 | −67.496 | −11.243 | 1.00 | 44.23 | C |
| ATOM | 3883 | O | ARG | A | 485 | 8.972 | −67.748 | −10.339 | 1.00 | 45.60 | O |
| ATOM | 3884 | N | ALA | A | 486 | 11.090 | −67.582 | −11.075 | 1.00 | 44.91 | N |
| ATOM | 3885 | CA | ALA | A | 486 | 11.635 | −68.205 | −9.824 | 1.00 | 45.17 | C |
| ATOM | 3886 | CB | ALA | A | 486 | 13.087 | −68.353 | −10.006 | 1.00 | 45.86 | C |
| ATOM | 3887 | C | ALA | A | 486 | 11.492 | −67.113 | −8.677 | 1.00 | 43.26 | C |
| ATOM | 3888 | O | ALA | A | 486 | 11.842 | −67.422 | −7.550 | 1.00 | 42.42 | O |
| ATOM | 3889 | N | CYS | A | 487 | 10.961 | −65.926 | −8.961 | 1.00 | 44.56 | N |
| ATOM | 3890 | CA | CYS | A | 487 | 10.781 | −64.890 | −7.928 | 1.00 | 41.73 | C |
| ATOM | 3891 | CB | CYS | A | 487 | 10.908 | −63.506 | −8.539 | 1.00 | 45.62 | C |
| ATOM | 3892 | SG | CYS | A | 487 | 12.591 | −62.954 | −8.776 | 1.00 | 43.43 | S |
| ATOM | 3893 | C | CYS | A | 487 | 9.431 | −64.907 | −7.233 | 1.00 | 41.89 | C |
| ATOM | 3894 | O | CYS | A | 487 | 9.058 | −63.998 | −6.622 | 1.00 | 39.84 | O |
| ATOM | 3895 | N | ARG | A | 488 | 8.675 | −66.000 | −7.358 | 1.00 | 39.16 | N |
| ATOM | 3896 | CA | ARG | A | 488 | 7.454 | −66.148 | −6.575 | 1.00 | 39.60 | C |
| ATOM | 3897 | CB | ARG | A | 488 | 6.735 | −67.449 | −6.932 | 1.00 | 39.96 | C |
| ATOM | 3898 | CG | ARG | A | 488 | 5.383 | −67.378 | −6.247 | 1.00 | 39.74 | C |
| ATOM | 3899 | CD | ARG | A | 488 | 4.452 | −68.385 | −7.105 | 1.00 | 41.72 | C |
| ATOM | 3900 | NE | ARG | A | 488 | 4.778 | −69.796 | −7.053 | 1.00 | 45.36 | N |
| ATOM | 3901 | CZ | ARG | A | 488 | 4.321 | −70.621 | −6.118 | 1.00 | 45.93 | C |
| ATOM | 3902 | NH1 | ARG | A | 488 | 3.524 | −70.156 | −5.149 | 1.00 | 42.85 | N |
| ATOM | 3903 | NH2 | ARG | A | 488 | 4.660 | −71.905 | −6.166 | 1.00 | 42.92 | N |
| ATOM | 3904 | C | ARG | A | 488 | 7.748 | −66.122 | −5.077 | 1.00 | 37.95 | C |
| ATOM | 3905 | O | ARG | A | 488 | 8.613 | −66.332 | −4.618 | 1.00 | 38.39 | O |
| ATOM | 3906 | N | GLU | A | 489 | 7.010 | −65.305 | −4.332 | 1.00 | 38.32 | N |
| ATOM | 3907 | CA | GLU | A | 489 | −2.861 | −65.286 | −2.861 | 1.00 | 41.60 | C |
| ATOM | 3908 | CB | GLU | A | 489 | 7.387 | −63.879 | −2.349 | 1.00 | 40.94 | C |
| ATOM | 3909 | CG | GLU | A | 489 | 8.726 | −63.357 | −2.858 | 1.00 | 52.25 | C |
| ATOM | 3910 | CD | GLU | A | 489 | 9.054 | −61.922 | −2.453 | 1.00 | 55.98 | C |
| ATOM | 3911 | OE1 | GLU | A | 489 | 8.242 | −61.256 | −1.762 | 1.00 | 51.77 | O |
| ATOM | 3912 | OE2 | GLU | A | 489 | 10.150 | −61.461 | −2.850 | 1.00 | 60.06 | O |
| ATOM | 3913 | C | GLU | A | 489 | 5.743 | −65.760 | −2.227 | 1.00 | 43.12 | C |
| ATOM | 3914 | O | GLU | A | 489 | 4.657 | −65.677 | −2.829 | 1.00 | 44.57 | O |
| ATOM | 3915 | N | LYS | A | 490 | 5.843 | −66.248 | −1.003 | 1.00 | 45.49 | N |
| ATOM | 3916 | CA | LYS | A | 490 | 4.668 | −66.568 | −0.216 | 1.00 | 50.61 | C |
| ATOM | 3917 | CB | LYS | A | 490 | 5.103 | −67.273 | 1.061 | 1.00 | 54.32 | C |
| ATOM | 3918 | CG | LYS | A | 490 | 5.942 | −66.381 | 1.960 | 1.00 | 61.26 | C |
| ATOM | 3919 | CD | LYS | A | 490 | 6.934 | −67.250 | 2.812 | 1.00 | 70.53 | C |
| ATOM | 3920 | CE | LYS | A | 490 | 7.663 | −66.272 | 3.723 | 1.00 | 73.83 | C |
| ATOM | 3921 | NZ | LYS | A | 490 | 8.899 | −66.270 | 4.298 | 1.00 | 79.38 | N |
| ATOM | 3922 | C | LYS | A | 490 | 3.915 | −65.264 | 0.105 | 1.00 | 46.60 | C |
| ATOM | 3923 | O | LYS | A | 490 | 4.462 | −64.268 | 0.001 | 1.00 | 44.43 | O |
| ATOM | 3924 | N | ASP | A | 491 | 2.647 | −65.391 | 0.457 | 1.00 | 44.56 | N |
| ATOM | 3925 | CA | ASP | A | 491 | 1.862 | −64.255 | 0.960 | 1.00 | 41.10 | C |
| ATOM | 3926 | CB | ASP | A | 491 | 2.485 | −63.694 | 2.247 | 1.00 | 47.12 | C |
| ATOM | 3927 | CG | ASP | A | 491 | 2.770 | −64.768 | 3.276 | 1.00 | 48.37 | C |
| ATOM | 3928 | OD1 | ASP | A | 491 | 2.122 | −65.841 | 3.231 | 1.00 | 50.02 | O |
| ATOM | 3929 | OD2 | ASP | A | 491 | 3.644 | −64.523 | 4.131 | 1.00 | 50.54 | O |
| ATOM | 3930 | C | ASP | A | 491 | 1.642 | −63.113 | −0.012 | 1.00 | 35.15 | C |
| ATOM | 3931 | O | ASP | A | 491 | 1.450 | −61.970 | 0.416 | 1.00 | 35.05 | O |
| ATOM | 3932 | N | ARG | A | 492 | 1.672 | −63.404 | −1.302 | 1.00 | 31.66 | N |
| ATOM | 3933 | CA | ARG | A | 492 | 1.314 | −62.414 | −2.311 | 1.00 | 34.92 | C |
| ATOM | 3934 | CB | ARG | A | 492 | 2.481 | −61.413 | −2.595 | 1.00 | 35.19 | C |
| ATOM | 3935 | CG | ARG | A | 492 | 3.396 | −61.786 | −3.757 | 1.00 | 34.73 | C |
| ATOM | 3936 | CD | ARG | A | 492 | 4.670 | −60.943 | −3.817 | 1.00 | 33.87 | C |
| ATOM | 3937 | NE | ARG | A | 492 | 5.618 | −61.463 | −4.805 | 1.00 | 31.81 | N |
| ATOM | 3938 | CZ | ARG | A | 492 | 6.690 | −60.799 | −5.253 | 1.00 | 35.40 | C |
| ATOM | 3939 | NH1 | ARG | A | 492 | 6.962 | −59.575 | −4.802 | 1.00 | 37.29 | N |
| ATOM | 3940 | NH2 | ARG | A | 492 | 7.507 | −61.354 | −6.143 | 1.00 | 28.75 | N |
| ATOM | 3941 | C | ARG | A | 492 | 0.875 | −63.161 | −3.563 | 1.00 | 36.40 | C |
| ATOM | 3942 | O | ARG | A | 492 | 1.112 | −64.371 | −3.679 | 1.00 | 42.82 | O |
| ATOM | 3943 | N | ASP | A | 493 | 0.236 | −62.447 | −4.489 | 1.00 | 35.83 | N |
| ATOM | 3944 | CA | ASP | A | 493 | −0.174 | −62.983 | −5.781 | 1.00 | 35.97 | C |
| ATOM | 3945 | CB | ASP | A | 493 | −0.495 | −61.836 | −6.751 | 1.00 | 36.54 | C |
| ATOM | 3946 | CG | ASP | A | 493 | −1.211 | −62.307 | −8.034 | 1.00 | 42.41 | C |
| ATOM | 3947 | CD1 | ASP | A | 493 | −0.871 | −63.374 | −8.611 | 1.00 | 38.14 | O |
| ATOM | 3948 | CD2 | ASP | A | 493 | −2.121 | −61.572 | −8.485 | 1.00 | 48.59 | O |
| ATOM | 3949 | C | ASP | A | 493 | 0.928 | −63.844 | −6.372 | 1.00 | 37.49 | C |
| ATOM | 3950 | O | ASP | A | 493 | 2.053 | −63.369 | −6.520 | 1.00 | 37.96 | O |
| ATOM | 3951 | N | PRO | A | 494 | 0.595 | −65.101 | −6.750 | 1.00 | 39.13 | N |
| ATOM | 3952 | CA | PRO | A | 494 | 1.551 | −66.068 | −7.300 | 1.00 | 36.87 | C |
| ATOM | 3953 | CB | PRO | A | 494 | 0.694 | −67.311 | −7.498 | 1.00 | 35.08 | C |
| ATOM | 3954 | CG | PRO | A | 494 | −0.409 | −67.138 | −6.522 | 1.00 | 37.17 | C |
| ATOM | 3955 | CD | PRO | A | 494 | −0.748 | −65.701 | −6.668 | 1.00 | 37.50 | C |
| ATOM | 3956 | C | PRO | A | 494 | 2.149 | −65.676 | −8.633 | 1.00 | 36.26 | C |
| ATOM | 3957 | O | PRO | A | 494 | 3.051 | −66.362 | −9.120 | 1.00 | 36.47 | O |
| ATOM | 3958 | N | GLU | A | 495 | 1.643 | −64.595 | −9.220 | 1.00 | 35.71 | N |
| ATOM | 3959 | CA | GLU | A | 495 | 2.104 | −64.331 | −10.511 | 1.00 | 34.83 | C |
| ATOM | 3960 | CB | GLU | A | 455 | 1.016 | −64.240 | −11.550 | 1.00 | 37.07 | C |
| ATOM | 3961 | CG | GLU | A | 495 | 0.760 | −65.646 | −12.160 | 1.00 | 41.88 | C |
| ATOM | 3962 | CD | GLU | A | 495 | −0.462 | −65.689 | −13.091 | 1.00 | 46.47 | C |
| ATOM | 3963 | OE1 | GLU | A | 495 | −1.521 | −65.112 | −12.765 | 1.00 | 45.81 | O |
| ATOM | 3964 | OE2 | GLU | A | 495 | −0.370 | −66.285 | −14.181 | 1.00 | 54.44 | O |
| ATOM | 3965 | C | GLU | A | 495 | 2.570 | −62.682 | −10.391 | 1.00 | 34.87 | C |
| ATOM | 3966 | O | GLU | A | 495 | 2.989 | −62.083 | −11.385 | 1.00 | 35.52 | O |
| ATOM | 3967 | N | ALA | A | 496 | 2.520 | −62.121 | −9.184 | 1.00 | 30.93 | N |
| ATOM | 3968 | CA | ALA | A | 496 | 3.054 | −60.776 | −8.977 | 1.00 | 31.20 | C |
| ATOM | 3969 | CB | ALA | A | 496 | 3.016 | −60.391 | −7.516 | 1.00 | 31.15 | C |
| ATOM | 3970 | C | ALA | A | 496 | 4.470 | −60.559 | −9.559 | 1.00 | 30.85 | C |
| ATOM | 3971 | O | ALA | A | 496 | 4.729 | −59.485 | −10.109 | 1.00 | 27.96 | O |
| ATOM | 3972 | N | GLN | A | 497 | 5.369 | −61.557 | −9.451 | 1.00 | 33.19 | N |
| ATOM | 3973 | CA | GLN | A | 497 | 6.779 | −61.460 | −9.983 | 1.00 | 30.30 | C |
| ATOM | 3974 | CB | GLN | A | 497 | 7.404 | −62.832 | −10.297 | 1.00 | 31.86 | C |
| ATOM | 3975 | CG | GLN | A | 497 | 7.138 | −64.021 | −9.423 | 1.00 | 33.67 | C |
| ATOM | 3976 | CD | GLN | A | 497 | 5.719 | −64.517 | −9.437 | 1.00 | 34.62 | C |
| ATOM | 3977 | OE1 | GLN | A | 497 | 4.907 | −64.067 | −8.656 | 1.00 | 36.96 | O |
| ATOM | 3978 | NE2 | GLN | A | 497 | 5.438 | −65.496 | −10.269 | 1.00 | 36.08 | N |
| ATOM | 3979 | C | GLN | A | 497 | 6.748 | −60.841 | −11.365 | 1.00 | 31.62 | C |
| ATOM | 3980 | O | GLN | A | 497 | 7.661 | −60.139 | −11.756 | 1.00 | 32.19 | O |
| ATOM | 3981 | N | PHE | A | 498 | 5.708 | −61.171 | −12.130 | 1.00 | 29.25 | N |
| ATOM | 3982 | CA | PHE | A | 498 | 5.687 | −60.860 | −13.554 | 1.00 | 29.07 | C |
| ATOM | 3983 | CB | PHE | A | 498 | 4.824 | −61.846 | −14.318 | 1.00 | 27.54 | C |
| ATOM | 3984 | CG | PHE | A | 498 | 5.413 | −63.213 | −14.416 | 1.00 | 28.29 | C |
| ATOM | 3985 | CD1 | PHE | A | 498 | 6.410 | −63.485 | −15.341 | 1.00 | 28.96 | C |
| ATOM | 3986 | CE1 | PHE | A | 498 | 6.951 | −64.758 | −15.447 | 1.00 | 28.43 | C |
| ATOM | 3987 | CZ | PHE | A | 498 | 6.468 | −65.776 | −14.644 | 1.00 | 30.85 | C |
| ATOM | 3988 | CE2 | PHE | A | 498 | 5.469 | −65.518 | −13.705 | 1.00 | 30.13 | C |
| ATOM | 3989 | CD2 | PHE | A | 498 | 4.951 | −64.245 | −13.592 | 1.00 | 28.44 | C |
| ATOM | 3990 | C | PHE | A | 498 | 5.145 | −59.485 | −13.837 | 1.00 | 27.10 | C |
| ATOM | 3991 | O | PHE | A | 498 | 5.143 | −59.059 | −14.984 | 1.00 | 26.18 | O |
| ATOM | 3992 | N | GLU | A | 499 | 4.712 | −58.784 | −12.798 | 1.00 | 25.45 | N |
| ATOM | 3993 | CA | GLU | A | 499 | 4.082 | −57.497 | −13.000 | 1.00 | 29.05 | C |
| ATOM | 3994 | CB | GLU | A | 499 | 2.645 | −57.502 | −12.428 | 1.00 | 28.23 | C |
| ATOM | 3995 | CG | GLU | A | 499 | 1.782 | −58.598 | −12.987 | 1.00 | 26.37 | C |
| ATOM | 3996 | CD | GLU | A | 499 | 0.279 | −58.362 | −12.759 | 1.00 | 28.75 | C |
| ATOM | 3997 | OE1 | GLU | A | 499 | −0.151 | −58.269 | −11.610 | 1.00 | 29.19 | O |
| ATOM | 3998 | OE2 | GLU | A | 499 | −0.486 | −59.282 | −13.731 | 1.00 | 28.50 | O |
| ATOM | 3999 | C | GLU | A | 499 | 4.909 | −56.358 | −12.400 | 1.00 | 30.60 | C |
| ATOM | 4000 | O | GLU | A | 499 | 4.329 | −55.395 | −11.880 | 1.00 | 31.00 | O |
| ATOM | 4001 | N | MET | A | 500 | 6.241 | −56.465 | −12.506 | 1.00 | 28.19 | N |
| ATOM | 4002 | CA | MET | A | 500 | 7.130 | −55.454 | −12.022 | 1.00 | 30.11 | C |
| ATOM | 4003 | CB | MET | A | 500 | 7.426 | −55.641 | −10.526 | 1.00 | 30.81 | C |
| ATOM | 4004 | CG | MET | A | 500 | 8.197 | −56.909 | −10.199 | 1.00 | 30.79 | C |
| ATOM | 4005 | SD | MET | A | 500 | 8.320 | −57.083 | −8.419 | 1.00 | 34.96 | O |
| ATOM | 4006 | CE | MET | A | 500 | 6.596 | −57.237 | −7.925 | 1.00 | 36.05 | C |
| ATOM | 4007 | C | MET | A | 500 | 8.424 | −55.474 | −12.830 | 1.00 | 32.05 | C |
| ATOM | 4008 | O | MET | A | 500 | 8.827 | −56.522 | −13.308 | 1.00 | 34.96 | O |
| ATOM | 4009 | N | PRO | A | 501 | 9.060 | −54.298 | −13.011 | 1.00 | 29.79 | N |
| ATOM | 4010 | CA | PRO | A | 501 | 10.265 | −54.200 | −13.810 | 1.00 | 29.34 | C |
| ATOM | 4011 | CB | PRO | A | 501 | 10.260 | −52.238 | −14.258 | 1.00 | 29.52 | C |
| ATOM | 4012 | CG | PRO | A | 501 | 9.508 | −52.004 | −13.184 | 1.00 | 29.84 | C |
| ATOM | 4013 | CD | PRO | A | 501 | 8.596 | −52.984 | −12.517 | 1.00 | 28.13 | C |
| ATOM | 4014 | C | PRO | A | 501 | 11.531 | −54.495 | −12.981 | 1.00 | 30.97 | C |
| ATOM | 4015 | O | PRO | A | 501 | 11.594 | −54.161 | −11.802 | 1.00 | 32.57 | O |
| ATOM | 4016 | N | TYR | A | 502 | 12.508 | −55.155 | −13.588 | 1.00 | 30.83 | N |
| ATOM | 4017 | CA | TYR | A | 502 | 13.753 | −55.463 | −12.902 | 1.00 | 30.46 | C |
| ATOM | 4018 | CB | TYR | A | 502 | 14.013 | −56.967 | −12.883 | 1.00 | 30.47 | C |
| ATOM | 4019 | CG | TYR | A | 502 | 12.905 | −57.764 | −12.195 | 1.00 | 31.04 | C |
| ATOM | 4020 | CD1 | TYR | A | 502 | 11.805 | −58.257 | −12.923 | 1.00 | 30.47 | C |
| ATOM | 4021 | CE1 | TYR | A | 502 | 10.790 | −58.987 | −12.285 | 1.00 | 31.23 | C |
| ATOM | 4022 | CZ | TYR | A | 502 | 10.900 | −59.252 | −10.922 | 1.00 | 31.45 | C |
| ATOM | 4023 | OH | TYR | A | 502 | 9.931 | −59.982 | −10.292 | 1.00 | 31.22 | O |
| ATOM | 4024 | CE2 | TYR | A | 502 | 11.997 | −58.803 | −10.196 | 1.00 | 28.76 | C |
| ATOM | 4025 | CD2 | TYR | A | 502 | 12.980 | −58.064 | −10.834 | 1.00 | 28.41 | C |
| ATOM | 4026 | C | TYR | A | 502 | 14.894 | −54.727 | −13.574 | 1.00 | 29.66 | C |
| ATOM | 4027 | O | TYR | A | 502 | 14.991 | −54.687 | −14.802 | 1.00 | 31.18 | O |
| ATOM | 4028 | N | VAL | A | 503 | 15.710 | −54.683 | −12.767 | 1.00 | 28.71 | N |
| ATOM | 4029 | CA | VAL | A | 503 | 17.034 | −53.648 | −13.207 | 1.00 | 30.59 | C |
| ATOM | 4030 | CB | VAL | A | 503 | 17.683 | −52.671 | −12.200 | 1.00 | 29.00 | C |
| ATOM | 4031 | CG1 | VAL | A | 503 | 19.157 | −52.420 | −12.555 | 1.00 | 30.35 | C |
| ATOM | 4032 | CG2 | VAL | A | 503 | 16.922 | −51.373 | −12.209 | 1.00 | 25.64 | C |
| ATOM | 4033 | C | VAL | A | 503 | 17.871 | −54.912 | −13.303 | 1.00 | 29.24 | C |
| ATOM | 4034 | O | VAL | A | 503 | 17.941 | −55.671 | −12.350 | 1.00 | 31.92 | O |
| ATOM | 4035 | N | VAL | A | 504 | 18.485 | −55.344 | −14.453 | 1.00 | 31.25 | N |
| ATOM | 4036 | CA | VAL | A | 504 | 19.204 | −56.382 | −14.697 | 1.00 | 31.48 | C |
| ATOM | 4037 | CB | VAL | A | 504 | 18.220 | −57.536 | −15.114 | 1.00 | 32.67 | C |
| ATOM | 4038 | CG1 | VAL | A | 504 | 17.170 | −57.653 | −16.138 | 1.00 | 29.79 | C |
| ATOM | 4039 | CG2 | VAL | A | 504 | 18.981 | −58.734 | −15.657 | 1.00 | 27.95 | C |
| ATOM | 4040 | C | VAL | A | 504 | 20.208 | −56.100 | −15.795 | 1.00 | 34.79 | C |
| ATOM | 4041 | O | VAL | A | 504 | 19.877 | −55.393 | −16.743 | 1.00 | 34.45 | O |
| ATOM | 4042 | N | ARG | A | 505 | 21.431 | −56.633 | −15.666 | 1.00 | 39.33 | N |
| ATOM | 4043 | CA | ARG | A | 505 | 22.403 | −56.608 | −16.766 | 1.00 | 38.74 | C |
| ATOM | 4044 | CB | ARG | A | 505 | 23.850 | −56.827 | −16.282 | 1.00 | 43.16 | C |
| ATOM | 4045 | CG | ARG | A | 505 | 24.861 | −55.887 | −16.938 | 1.00 | 45.32 | C |
| ATOM | 4046 | CD | ARG | A | 505 | 25.335 | −56.205 | −18.354 | 1.00 | 45.45 | C |
| ATOM | 4047 | NE | ARG | A | 505 | 24.305 | −56.368 | −19.394 | 1.00 | 48.77 | N |
| ATOM | 4048 | CZ | ARG | A | 505 | 23.803 | −55.401 | −20.172 | 1.00 | 49.01 | C |
| ATOM | 4049 | NH1 | ARG | A | 505 | 22.894 | −55.708 | −21.091 | 1.00 | 46.88 | N |
| ATOM | 4050 | NH2 | ARG | A | 505 | 24.182 | −54.132 | −20.038 | 1.00 | 50.01 | N |
| ATOM | 4051 | C | ARG | A | 505 | 22.061 | −57.725 | −17.732 | 1.00 | 40.57 | C |
| ATOM | 4052 | O | ARG | A | 505 | 22.560 | −58.848 | −17.596 | 1.00 | 40.29 | O |
| ATOM | 4053 | N | LEU | A | 506 | 21.213 | −57.418 | −18.707 | 1.00 | 40.02 | N |
| ATOM | 4054 | CA | LEU | A | 506 | 20.765 | −58.410 | −19.662 | 1.00 | 39.25 | C |
| ATOM | 4055 | CB | LEU | A | 506 | 19.883 | −57.744 | −20.712 | 1.00 | 39.06 | C |
| ATOM | 4056 | CG | LEU | A | 506 | 18.672 | −56.918 | −20.261 | 1.00 | 37.20 | C |
| ATOM | 4057 | CD1 | LEU | A | 506 | 18.197 | −56.151 | −21.466 | 1.00 | 36.14 | C |
| ATOM | 4058 | CD2 | LEU | A | 506 | 17.567 | −57.792 | −19.705 | 1.00 | 33.90 | C |
| ATOM | 4059 | C | LEU | A | 506 | 21.968 | −59.098 | −20.328 | 1.00 | 40.41 | C |
| ATOM | 4060 | O | LEU | A | 506 | 22.756 | −58.455 | −20.993 | 1.00 | 41.20 | O |
| ATOM | 4061 | N | HIS | A | 507 | 22.090 | −60.408 | −20.158 | 1.00 | 39.86 | N |
| ATOM | 4062 | CA | HIS | A | 507 | 23.206 | −61.123 | −20.726 | 1.00 | 41.33 | C |
| ATOM | 4063 | CB | HIS | A | 507 | 24.111 | −61.615 | −19.618 | 1.00 | 41.01 | C |
| ATOM | 4064 | CG | HIS | A | 507 | 25.409 | −62.156 | −20.117 | 1.00 | 44.77 | C |
| ATOM | 4065 | ND1 | HIS | A | 507 | 26.253 | −61.427 | −20.930 | 1.00 | 45.57 | N |
| ATOM | 4066 | CE1 | HIS | A | 507 | 27.316 | −62.155 | −21.217 | 1.00 | 45.15 | C |
| ATOM | 4067 | NE2 | HIS | A | 507 | 27.193 | −63.326 | −20.619 | 1.00 | 44.56 | N |
| ATOM | 4068 | CD2 | HIS | A | 507 | 26.006 | −63.355 | −19.929 | 1.00 | 43.35 | C |
| ATOM | 4069 | C | HIS | A | 507 | 22.832 | −62.276 | −21.678 | 1.00 | 44.65 | C |
| ATOM | 4070 | O | HIS | A | 507 | 23.106 | −62.204 | −22.865 | 1.00 | 44.60 | O |
| ATOM | 4071 | N | ASN | A | 508 | 22.245 | −63.349 | −21.157 | 1.00 | 45.23 | N |
| ATOM | 4072 | CA | ASN | A | 508 | 21.799 | −64.451 | −22.006 | 1.00 | 45.67 | C |
| ATOM | 4073 | CB | ASN | A | 508 | 21.888 | −65.793 | −21.234 | 1.00 | 44.08 | C |
| ATOM | 4074 | CG | ASN | A | 508 | 21.436 | −67.003 | −22.053 | 1.00 | 40.91 | C |
| ATOM | 4075 | OD1 | ASN | A | 508 | 21.490 | −67.031 | −23.287 | 1.00 | 39.33 | O |
| ATOM | 4076 | ND2 | ASN | A | 508 | 20.990 | −68.016 | −21.349 | 1.00 | 41.66 | N |
| ATOM | 4077 | C | ASN | A | 508 | 20.385 | −64.127 | −22.538 | 1.00 | 45.54 | C |
| ATOM | 4078 | O | ASN | A | 508 | 19.371 | −64.516 | −21.951 | 1.00 | 43.77 | O |
| ATOM | 4079 | N | PHE | A | 509 | 20.335 | −63.365 | −23.626 | 1.00 | 43.32 | N |
| ATOM | 4080 | CA | PHE | A | 509 | 19.062 | −62.935 | −24.203 | 1.00 | 44.36 | C |
| ATOM | 4081 | CB | PHE | A | 509 | 18.508 | −61.683 | −23.477 | 1.00 | 42.00 | C |
| ATOM | 4082 | CG | PHE | A | 509 | 19.154 | −60.378 | −23.887 | 1.00 | 38.12 | C |
| ATOM | 4083 | CD1 | PHE | A | 509 | 20.506 | −60.317 | −23.606 | 1.00 | 38.89 | C |
| ATOM | 4084 | CE1 | PHE | A | 509 | 21.095 | −58.909 | −23.987 | 1.00 | 36.99 | C |
| ATOM | 4085 | CZ | PHE | A | 509 | 20.336 | −57.943 | −24.642 | 1.00 | 37.52 | C |
| ATOM | 4086 | CE2 | PHE | A | 509 | 18.984 | −58.193 | −24.908 | 1.00 | 37.76 | C |
| ATOM | 4087 | CD2 | PHE | A | 509 | 18.412 | −59.400 | −24.531 | 1.00 | 36.25 | C |
| ATOM | 4088 | C | PHE | A | 509 | 19.215 | −62.659 | −25.687 | 1.00 | 44.46 | C |
| ATOM | 4089 | O | PHE | A | 509 | 20.332 | −62.559 | −26.193 | 1.00 | 47.70 | O |
| ATOM | 4090 | N | HIS | A | 510 | 18.089 | −62.540 | −26.379 | 1.00 | 42.40 | N |
| ATOM | 4091 | CA | HIS | A | 510 | 18.089 | −62.135 | −27.774 | 1.00 | 43.17 | C |
| ATOM | 4092 | CB | HIS | A | 510 | 17.621 | −63.287 | −28.683 | 1.00 | 39.61 | C |
| ATOM | 4093 | CG | HIS | A | 510 | 17.634 | −62.948 | −30.137 | 1.00 | 37.88 | C |
| ATOM | 4094 | ND1 | HIS | A | 510 | 16.864 | −63.622 | −31.065 | 1.00 | 38.99 | N |
| ATOM | 4095 | CE1 | HIS | A | 510 | 17.083 | −63.105 | −32.264 | 1.00 | 42.21 | C |
| ATOM | 4096 | NE2 | HIS | A | 510 | 17.963 | −62.118 | −32.148 | 1.00 | 40.22 | N |
| ATOM | 4097 | CD2 | HIS | A | 510 | 18.317 | −61.996 | −30.826 | 1.00 | 39.79 | C |
| ATOM | 4098 | C | HIS | A | 510 | 17.185 | −60.926 | −27.912 | 1.00 | 45.60 | C |
| ATOM | 4099 | O | HIS | A | 510 | 16.029 | −60.953 | −27.458 | 1.00 | 45.31 | O |
| ATOM | 4100 | N | GLN | A | 511 | 17.719 | −59.857 | −28.502 | 1.00 | 45.50 | N |
| ATOM | 4101 | CA | GLN | A | 511 | 16.911 | −58.680 | −28.753 | 1.00 | 43.67 | C |
| ATOM | 4102 | CB | GLN | A | 511 | 17.737 | −57.420 | −28.721 | 1.00 | 42.21 | C |
| ATOM | 4103 | CG | GLN | A | 511 | 16.874 | −56.224 | −28.396 | 1.00 | 43.04 | C |
| ATOM | 4104 | CD | GLN | A | 511 | 17.653 | −54.935 | −28.366 | 1.00 | 42.68 | C |
| ATOM | 4105 | OE1 | GLN | A | 511 | 17.941 | −54.345 | −29.419 | 1.00 | 39.62 | O |
| ATOM | 4106 | NE2 | GLN | A | 511 | 17.980 | −54.472 | −27.154 | 1.00 | 41.26 | N |
| ATOM | 4107 | C | GLN | A | 511 | 16.206 | −58.784 | −30.085 | 1.00 | 46.68 | C |
| ATOM | 4108 | O | GLN | A | 511 | 16.852 | −58.863 | −31.140 | 1.00 | 42.58 | O |
| ATOM | 4109 | N | LEU | A | 512 | 14.872 | −58.771 | −30.025 | 1.00 | 45.83 | N |
| ATOM | 4110 | CA | LEU | A | 512 | 14.041 | −59.100 | −31.179 | 1.00 | 40.57 | C |
| ATOM | 4111 | CB | LEU | A | 512 | 12.719 | −59.670 | −30.705 | 1.00 | 39.95 | C |
| ATOM | 4112 | CG | LEU | A | 512 | 12.560 | −61.164 | −30.411 | 1.00 | 40.80 | C |
| ATOM | 4113 | CD1 | LEU | A | 512 | 13.837 | −61.958 | −30.128 | 1.00 | 40.74 | C |
| ATOM | 4114 | CD2 | LEU | A | 512 | 11.549 | −61.331 | −29.296 | 1.00 | 38.75 | C |
| ATOM | 4115 | C | LEU | A | 512 | 13.815 | −57.952 | −32.166 | 1.00 | 41.97 | C |
| ATOM | 4116 | O | LEU | A | 512 | 13.484 | −58.212 | −33.327 | 1.00 | 41.41 | O |
| ATOM | 4117 | N | SER | A | 513 | 13.974 | −56.695 | −31.722 | 1.00 | 39.59 | N |
| ATOM | 4118 | CA | SER | A | 513 | 13.777 | −55.530 | −32.602 | 1.00 | 37.05 | C |
| ATOM | 4119 | CB | SER | A | 513 | 12.286 | −55.213 | −32.764 | 1.00 | 39.61 | C |
| ATOM | 4120 | OG | SER | A | 513 | 12.050 | −54.147 | −33.697 | 1.00 | 36.95 | O |
| ATOM | 4121 | C | SER | A | 513 | 14.536 | −54.299 | −32.083 | 1.00 | 41.65 | C |
| ATOM | 4122 | O | SER | A | 513 | 14.913 | −54.242 | −30.910 | 1.00 | 44.45 | O |
| ATOM | 4123 | N | ALA | A | 514 | 14.762 | −53.307 | −32.944 | 1.00 | 40.16 | N |
| ATOM | 4124 | CA | ALA | A | 514 | 15.467 | −52.093 | −32.520 | 1.00 | 42.63 | C |
| ATOM | 4125 | CB | ALA | A | 514 | 15.704 | −51.175 | −33.713 | 1.00 | 37.56 | C |
| ATOM | 4126 | C | ALA | A | 514 | 14.644 | −51.359 | −31.447 | 1.00 | 45.31 | C |
| ATOM | 4127 | O | ALA | A | 514 | 13.410 | −51.299 | −31.560 | 1.00 | 47.27 | O |
| ATOM | 4128 | N | PRO | A | 515 | 15.316 | −50.777 | −30.423 | 1.00 | 44.84 | N |
| ATOM | 4129 | CA | PRO | A | 515 | 14.586 | −50.022 | −29.388 | 1.00 | 41.50 | C |
| ATOM | 4130 | CB | PRO | A | 515 | 15.699 | 49.462 | −28.502 | 1.00 | 41.79 | C |
| ATOM | 4131 | CG | PRO | A | 515 | 16.843 | −50.420 | −28.680 | 1.00 | 40.80 | C |
| ATOM | 4132 | CD | PRO | A | 515 | 16.762 | −50.836 | −30.133 | 1.00 | 42.94 | C |
| ATOM | 4133 | C | PRO | A | 515 | 13.796 | −48.896 | −29.997 | 1.00 | 40.86 | C |
| ATOM | 4134 | O | PRO | A | 515 | 14.194 | −48.361 | −31.026 | 1.00 | 44.27 | O |
| ATOM | 4135 | N | GLN | A | 516 | 12.665 | −48.549 | −29.396 | 1.00 | 39.37 | N |
| ATOM | 4136 | CA | GLN | A | 516 | 11.897 | −47.394 | −29.894 | 1.00 | 39.21 | C |
| ATOM | 4137 | CB | GLN | A | 516 | 10.652 | −47.842 | −30.674 | 1.00 | 38.84 | C |
| ATOM | 4138 | CG | GLN | A | 516 | 10.942 | −48.305 | −32.102 | 1.00 | 38.41 | C |
| ATOM | 4139 | CD | GLN | A | 516 | 9.687 | −48.727 | −32.842 | 1.00 | 42.85 | C |
| ATOM | 4140 | OE1 | GLN | A | 516 | 8.667 | −48.010 | −32.831 | 1.00 | 42.94 | O |
| ATOM | 4141 | NE2 | GLN | A | 516 | 9.741 | −49.902 | −33.479 | 1.00 | 38.68 | N |
| ATOM | 4142 | C | GLN | A | 516 | 11.548 | −46.479 | −28.730 | 1.00 | 36.08 | C |
| ATOM | 4143 | O | GLN | A | 516 | 11.438 | −46.959 | −27.604 | 1.00 | 36.64 | O |
| ATOM | 4144 | N | PRO | A | 517 | 11.445 | −45.165 | −28.983 | 1.00 | 31.09 | N |
| ATOM | 4145 | CA | PRO | A | 517 | 11.191 | −44.228 | −27.911 | 1.00 | 32.73 | C |
| ATOM | 4146 | CB | PRO | A | 517 | 11.123 | −42.856 | −28.627 | 1.00 | 32.81 | C |
| ATOM | 4147 | CG | PRO | A | 517 | 11.039 | −43.157 | −30.087 | 1.00 | 31.16 | C |
| ATOM | 4148 | CD | PRO | A | 517 | 11.713 | −44.485 | −30.262 | 1.00 | 31.86 | C |
| ATOM | 4149 | C | PRO | A | 517 | 9.856 | −44.544 | −27.267 | 1.00 | 35.76 | C |
| ATOM | 4150 | O | PRO | A | 517 | 8.943 | −44.984 | −27.952 | 1.00 | 35.37 | O |
| ATOM | 4151 | N | CYS | A | 518 | 9.754 | −44.341 | −25.958 | 1.00 | 36.48 | N |
| ATOM | 4152 | CA | CYS | A | 518 | 8.532 | −44.660 | −25.261 | 1.00 | 39.50 | C |
| ATOM | 4153 | CB | CYS | A | 518 | 8.780 | −45.766 | −24.226 | 1.00 | 39.12 | C |
| ATOM | 4154 | SG | CYS | A | 518 | 7.268 | −46.328 | −23.389 | 1.00 | 36.16 | S |
| ATOM | 4155 | C | CYS | A | 518 | 7.917 | −43.378 | −24.654 | 1.00 | 39.96 | C |
| ATOM | 4156 | O | CYS | A | 518 | 7.022 | −42.780 | −25.248 | 1.00 | 36.14 | O |
| ATOM | 4157 | N | PHE | A | 519 | 8.414 | −42.957 | −23.491 | 1.00 | 39.47 | N |
| ATOM | 4158 | CA | PHE | A | 519 | 7.909 | −41.164 | −22.817 | 1.00 | 36.32 | C |
| ATOM | 4159 | CB | PHE | A | 519 | 7.529 | −42.067 | −21.369 | 1.00 | 35.86 | C |
| ATOM | 4160 | CG | PHE | A | 519 | 6.456 | −43.099 | −21.226 | 1.00 | 35.98 | C |
| ATOM | 4161 | CD1 | PHE | A | 519 | 5.392 | −43.157 | −22.135 | 1.00 | 36.14 | C |
| ATOM | 4162 | CE1 | PHE | A | 519 | 4.396 | −44.111 | −22.005 | 1.00 | 34.99 | C |
| ATOM | 4163 | CZ | PHE | A | 519 | 4.444 | −45.008 | −20.953 | 1.00 | 35.02 | C |
| ATOM | 4164 | CE2 | PHE | A | 519 | 5.493 | −44.951 | −20.033 | 1.00 | 36.12 | C |
| ATOM | 4165 | CD2 | PHE | A | 519 | 6.490 | −43.995 | −20.172 | 1.00 | 34.55 | C |
| ATOM | 4166 | C | PHE | A | 519 | 8.967 | −40.705 | −22.798 | 1.00 | 35.09 | C |
| ATOM | 4167 | O | PHE | A | 519 | 10.177 | −41.016 | −22.683 | 1.00 | 37.07 | O |
| ATOM | 4168 | N | ATHR | A | 520 | 8.527 | −39.455 | −22.879 | 0.50 | 33.56 | N |
| ATOM | 4169 | N | BTHR | A | 520 | 8.545 | −39.452 | −22.916 | 0.50 | 34.65 | N |
| ATOM | 4170 | CA | ATHR | A | 520 | 9.444 | −38.323 | −22.879 | 0.50 | 33.17 | C |
| ATOM | 4171 | CA | BTHR | A | 520 | 9.492 | −38.343 | −22.864 | 0.50 | 35.14 | C |
| ATOM | 4172 | CB | ATHR | A | 520 | 9.646 | −37.791 | −24.317 | 0.50 | 32.87 | C |
| ATOM | 4173 | CB | BTHR | A | 520 | 9.873 | −37.849 | −24.286 | 0.50 | 36.15 | C |
| ATOM | 4174 | OG1 | ATHR | A | 520 | 10.332 | −38.781 | −25.096 | 0.50 | 30.07 | O |
| ATOM | 4175 | OG1 | BTHR | A | 520 | 10.858 | −36.807 | −24.199 | 0.50 | 36.12 | O |
| ATOM | 4176 | CG2 | ATHR | A | 520 | 10.452 | −36.486 | −24.319 | 0.50 | 32.50 | C |
| ATOM | 4177 | CG2 | BTHR | A | 520 | 8.648 | −37.341 | −25.040 | 0.50 | 35.78 | C |
| ATOM | 4178 | C | ATHR | A | 520 | 8.985 | −37.208 | −21.937 | 0.50 | 33.94 | C |
| ATOM | 4179 | C | BTHR | A | 520 | 9.007 | −37.197 | −21.971 | 0.50 | 35.15 | C |
| ATOM | 4180 | C | ATHR | A | 520 | 7.794 | −36.857 | −21.898 | 0.50 | 35.23 | O |
| ATOM | 4181 | O | BTHR | A | 520 | 7.824 | −36.823 | −21.996 | 0.50 | 36.56 | O |
| ATOM | 4182 | N | PHE | A | 521 | 9.929 | −36.662 | −21.174 | 1.00 | 33.75 | N |
| ATOM | 4183 | CA | PHE | A | 521 | 9.661 | −35.513 | −20.303 | 1.00 | 33.24 | C |
| ATOM | 4184 | CB | PHE | A | 521 | 9.693 | −35.932 | −18.815 | 1.00 | 30.74 | C |
| ATOM | 4185 | CG | PHE | A | 521 | 8.607 | −36.912 | −18.462 | 1.00 | 32.01 | C |
| ATOM | 4186 | CD1 | PHE | A | 521 | 7.355 | −36.460 | −18.015 | 1.00 | 30.92 | C |
| ATOM | 4187 | CE1 | PHE | A | 521 | 6.328 | −37.358 | −17.745 | 1.00 | 30.82 | C |
| ATOM | 4188 | CZ | PHE | A | 521 | 6.529 | −38.716 | −17.939 | 1.00 | 32.01 | C |
| ATOM | 4189 | CE2 | PHE | A | 521 | 7.773 | −39.176 | −18.398 | 1.00 | 32.28 | C |
| ATOM | 4190 | CD2 | PHE | A | 521 | 8.795 | −38.276 | −18.663 | 1.00 | 29.37 | C |
| ATOM | 4191 | C | PHE | A | 521 | 10.649 | −34.393 | −20.568 | 1.00 | 36.59 | C |
| ATOM | 4192 | C | PHE | A | 521 | 11.829 | −34.649 | −20.869 | 1.00 | 40.93 | O |
| ATOM | 4193 | N | SER | A | 522 | 10.167 | −33.159 | −20.427 | 1.00 | 39.78 | N |
| ATOM | 4194 | CA | SER | A | 522 | 10.973 | −31.937 | −20.583 | 1.00 | 43.55 | C |
| ATOM | 4195 | CB | SER | A | 522 | 10.441 | −31.099 | −21.751 | 1.00 | 40.56 | C |
| ATOM | 4196 | CG | SER | A | 522 | 10.918 | −31.605 | −22.988 | 1.00 | 43.57 | O |
| ATOM | 4197 | C | SER | A | 522 | 10.945 | −31.100 | −19.316 | 1.00 | 41.90 | C |
| ATOM | 4198 | O | SER | A | 522 | 9.923 | −31.042 | −18.628 | 1.00 | 45.51 | O |
| ATOM | 4199 | N | HIS | A | 523 | 12.058 | −30.435 | −19.012 | 1.00 | 43.57 | N |
| ATOM | 4200 | CA | HIS | A | 523 | 12.131 | −29.590 | −17.814 | 1.00 | 43.54 | C |
| ATOM | 4201 | CB | HIS | A | 523 | 12.829 | −30.341 | −16.675 | 1.00 | 42.00 | C |
| ATOM | 4202 | CG | HIS | A | 523 | 12.087 | −31.566 | −16.235 | 1.00 | 47.01 | C |
| ATOM | 4203 | ND1 | HIS | A | 523 | 10.868 | −31.506 | −15.592 | 1.00 | 44.65 | N |
| ATOM | 4204 | OE1 | HIS | A | 523 | 10.442 | −32.734 | −15.352 | 1.00 | 47.71 | C |
| ATOM | 4205 | NE2 | HIS | A | 523 | 11.341 | −33.587 | −15.813 | 1.00 | 46.32 | N |
| ATOM | 4206 | CD2 | HIS | A | 523 | 12.376 | −32.882 | −16.374 | 1.00 | 45.58 | C |
| ATOM | 4207 | C | HIS | A | 523 | 12.794 | −28.241 | −18.084 | 1.00 | 44.54 | C |
| ATOM | 4208 | O | HIS | A | 523 | 13.838 | −28.189 | −18.728 | 1.00 | 48.30 | O |
| ATOM | 4209 | N | PRO | A | 524 | 12.194 | −27.139 | −17.599 | 1.00 | 46.37 | N |
| ATOM | 4210 | CA | PRO | A | 524 | 10.916 | −27.030 | −16.896 | 1.00 | 46.46 | C |
| ATOM | 4211 | CB | PRO | A | 524 | 10.834 | −25.532 | −16.554 | 1.00 | 48.85 | C |
| ATOM | 4212 | CG | PRO | A | 524 | 11.503 | −24.866 | −17.569 | 1.00 | 46.66 | C |
| ATOM | 4213 | CD | PRO | A | 524 | 12.861 | −25.823 | −17.694 | 1.00 | 45.78 | C |
| ATOM | 4214 | C | PRO | A | 524 | 9.740 | −27.389 | −17.784 | 1.00 | 50.51 | C |
| ATOM | 4215 | O | PRO | A | 524 | 9.874 | −27.461 | −19.013 | 1.00 | 51.04 | O |
| ATOM | 4216 | N | ASN | A | 525 | 8.588 | −27.585 | −17.160 | 1.00 | 54.63 | N |
| ATOM | 4217 | CA | ASN | A | 525 | 7.393 | −28.005 | −17.870 | 1.00 | 62.29 | C |
| ATOM | 4218 | CB | ASN | A | 525 | 6.746 | −29.178 | −17.123 | 1.00 | 61.80 | C |
| ATOM | 4219 | CG | ASN | A | 525 | 5.631 | −29.883 | −17.904 | 1.00 | 62.77 | C |
| ATOM | 4220 | OD1 | ASN | A | 525 | 5.123 | −30.897 | −17.459 | 1.00 | 61.63 | O |
| ATOM | 4221 | ND2 | ASN | A | 525 | 5.233 | −29.299 | −19.045 | 1.00 | 55.86 | N |
| ATOM | 4222 | C | ASN | A | 525 | 6.450 | −26.818 | −18.008 | 1.00 | 68.35 | C |
| ATOM | 4223 | O | ASN | A | 525 | 5.790 | −26.401 | −17.054 | 1.00 | 66.72 | O |
| ATOM | 4224 | N | ARG | A | 526 | 6.403 | −26.275 | −19.217 | 1.00 | 80.73 | N |
| ATOM | 4225 | CA | ARG | A | 526 | 5.609 | −25.081 | −19.508 | 1.00 | 90.30 | C |
| ATOM | 4226 | CB | ARG | A | 526 | 6.272 | −24.254 | −20.627 | 1.00 | 92.13 | C |
| ATOM | 4227 | CG | ARG | A | 526 | 7.027 | −25.057 | −21.690 | 1.00 | 92.81 | C |
| ATOM | 4228 | CD | ARG | A | 526 | 8.126 | −24.237 | −22.367 | 1.00 | 90.84 | C |
| ATOM | 4229 | NE | ARG | A | 576 | 9.273 | −23.967 | −21.487 | 1.00 | 95.28 | N |
| ATOM | 4230 | CZ | ARG | A | 526 | 9.427 | −22.880 | −20.719 | 1.00 | 92.60 | C |
| ATOM | 4231 | NH1 | ARG | A | 526 | 8.506 | −21.920 | −20.697 | 1.00 | 89.70 | N |
| ATOM | 4232 | NH2 | ARG | A | 526 | 10.513 | −22.748 | −19.961 | 1.00 | 82.19 | N |
| ATOM | 4233 | C | ARG | A | 526 | 4.117 | −25.348 | −19.797 | 1.00 | 95.58 | C |
| ATOM | 4234 | O | ARG | A | 526 | 3.380 | −24.429 | −20.167 | 1.00 | 100.44 | O |
| ATOM | 4235 | N | ASP | A | 527 | 3.682 | −26.598 | −19.615 | 1.00 | 95.61 | N |
| ATOM | 4236 | CA | ASP | A | 527 | 2.272 | −26.982 | −19.762 | 1.00 | 95.35 | C |
| ATOM | 4237 | CB | ASP | A | 527 | 2.097 | −28.494 | −19.564 | 1.00 | 97.26 | C |
| ATOM | 4238 | CG | ASP | A | 527 | 2.080 | −29.264 | −20.877 | 1.00 | 98.31 | C |
| ATOM | 4239 | OD1 | ASP | A | 527 | 1.740 | −28.659 | −21.919 | 1.00 | 100.45 | O |
| ATOM | 4240 | OD2 | ASP | A | 527 | 2.394 | −30.477 | −20.864 | 1.00 | 90.93 | O |
| ATOM | 4241 | C | ASP | A | 527 | 1.360 | −26.230 | −18.787 | 1.00 | 95.47 | C |
| ATOM | 4242 | O | ASP | A | 527 | 1.672 | −26.145 | −17.594 | 1.00 | 89.43 | O |
| ATOM | 4243 | N | PRO | A | 528 | 0.229 | −25.685 | −19.290 | 1.00 | 98.66 | N |
| ATOM | 4244 | CA | PRO | A | 528 | −0.692 | −24.960 | −18.401 | 1.00 | 97.50 | C |
| ATOM | 4245 | CB | PRO | A | 528 | −1.807 | −24.469 | −19.344 | 1.00 | 95.96 | C |
| ATOM | 4246 | CG | PRO | A | 528 | −1.704 | −25.315 | −20.570 | 1.00 | 99.62 | C |
| ATOM | 4247 | CD | PRO | A | 528 | −0.272 | −25.765 | −20.678 | 1.00 | 97.80 | C |
| ATOM | 4248 | C | PRO | A | 528 | −1.239 | −25.911 | −17.345 | 1.00 | 97.70 | C |
| ATOM | 4249 | O | PRO | A | 528 | −1.247 | −25.586 | −16.155 | 1.00 | 93.98 | O |
| ATOM | 4250 | N | MET | A | 529 | −1.687 | −27.277 | −17.804 | 1.00 | 101.46 | N |
| ATOM | 4251 | CA | MET | A | 529 | −2.020 | −28.199 | −16.946 | 1.00 | 100.10 | C |
| ATOM | 4252 | CB | MET | A | 529 | −3.377 | −28.810 | −17.339 | 1.00 | 111.36 | C |
| ATOM | 4253 | CG | MET | A | 529 | −4.620 | −27.954 | −17.072 | 1.00 | 113.77 | C |
| ATOM | 4254 | SD | MET | A | 529 | −5.132 | −26.777 | −18.355 | 1.00 | 118.43 | S |
| ATOM | 4255 | CE | MET | A | 529 | −5.144 | −27.758 | −19.860 | 1.00 | 118.56 | C |
| ATOM | 4256 | C | MET | A | 529 | −0.905 | −29.228 | −17.131 | 1.00 | 93.15 | C |
| ATOM | 4257 | O | MET | A | 529 | −0.669 | −29.718 | −18.244 | 1.00 | 91.84 | O |
| ATOM | 4258 | N | ILE | A | 530 | −0.197 | −29.532 | −16.051 | 1.00 | 79.63 | N |
| ATOM | 4259 | CA | ILE | A | 530 | 0.832 | −30.554 | −16.112 | 1.00 | 72.45 | C |
| ATOM | 4260 | CB | ILE | A | 530 | 1.910 | −30.354 | −15.025 | 1.00 | 76.57 | C |
| ATOM | 4261 | CG1 | ILE | A | 530 | 2.721 | −29.173 | −15.291 | 1.00 | 74.40 | C |
| ATOM | 4262 | CD1 | ILE | A | 530 | 3.450 | −28.532 | −14.073 | 1.00 | 69.02 | C |
| ATOM | 4263 | CG2 | ILE | A | 530 | 2.821 | −31.585 | −14.934 | 1.00 | 76.35 | C |
| ATOM | 4264 | C | ILE | A | 530 | 0.188 | −31.927 | −15.940 | 1.00 | 66.86 | C |
| ATOM | 4265 | O | ILE | A | 530 | −0.444 | −32.195 | −14.912 | 1.00 | 62.93 | O |
| ATOM | 4266 | N | ASP | A | 531 | 0.342 | −32.787 | −16.949 | 1.00 | 56.94 | N |
| ATOM | 4267 | CA | ASP | A | 531 | 0.004 | −34.199 | −16.785 | 1.00 | 51.67 | C |
| ATOM | 4268 | CB | ASP | A | 531 | −1.254 | −34.554 | −17.557 | 1.00 | 53.38 | C |
| ATOM | 4269 | CG | ASP | A | 531 | −1.838 | −35.362 | −17.110 | 1.00 | 59.11 | C |
| ATOM | 4270 | OD1 | ASP | A | 531 | −1.152 | −36.684 | −16.362 | 1.00 | 62.03 | O |
| ATOM | 4271 | OD2 | ASP | A | 531 | −2.979 | −36.174 | −17.500 | 1.00 | 62.19 | O |
| ATOM | 4272 | C | ASP | A | 531 | 1.157 | −35.202 | −17.079 | 1.00 | 42.39 | C |
| ATOM | 4273 | O | ASP | A | 531 | 1.644 | −35.329 | −18.198 | 1.00 | 35.69 | O |
| ATOM | 4274 | N | ASN | A | 532 | 1.574 | −35.921 | −16.052 | 1.00 | 37.60 | N |
| ATOM | 4275 | CA | ASN | A | 532 | 2.673 | −36.875 | −16.215 | 1.00 | 34.42 | C |
| ATOM | 4276 | CB | ASN | A | 532 | 3.589 | −36.862 | −15.010 | 1.00 | 33.17 | C |
| ATOM | 4277 | CG | ASN | A | 532 | 4.436 | −35.601 | −14.978 | 1.00 | 35.29 | C |
| ATOM | 4278 | OD1 | ASN | A | 532 | 4.699 | −34.996 | −16.037 | 1.00 | 33.71 | O |
| ATOM | 4279 | ND2 | ASN | A | 532 | 4.850 | −35.183 | −13.789 | 1.00 | 34.82 | N |
| ATOM | 4280 | C | ASN | A | 532 | 2.304 | −38.274 | −16.663 | 1.00 | 33.58 | C |
| ATOM | 4281 | O | ASN | A | 532 | 3.199 | −39.029 | −17.029 | 1.00 | 30.98 | O |
| ATOM | 4282 | N | ASN | A | 533 | 0.996 | −38.390 | −16.676 | 1.00 | 29.27 | N |
| ATOM | 4283 | CA | ASN | A | 533 | 0.513 | −39.852 | −17.201 | 1.00 | 29.95 | C |
| ATOM | 4284 | CB | ASN | A | 533 | −0.977 | −40.021 | −16.924 | 1.00 | 33.75 | C |
| ATOM | 4285 | CG | ASN | A | 533 | −1.294 | −40.039 | −15.450 | 1.00 | 34.04 | C |
| ATOM | 4286 | OD1 | ASN | A | 533 | −0.487 | −40.447 | −14.615 | 1.00 | 34.90 | O |
| ATOM | 4287 | ND2 | ASN | A | 533 | −2.468 | −39.590 | −15.124 | 1.00 | 35.65 | N |
| ATOM | 4288 | C | ASN | A | 533 | 0.742 | −39.979 | −18.698 | 1.00 | 31.47 | C |
| ATOM | 4289 | O | ASN | A | 533 | 0.837 | −38.991 | −19.434 | 1.00 | 29.88 | O |
| ATOM | 4290 | N | ARG | A | 534 | 0.829 | −41.212 | −19.171 | 1.00 | 31.06 | N |
| ATOM | 4291 | CA | ARG | A | 534 | 1.148 | −41.423 | −20.568 | 1.00 | 29.30 | C |
| ATOM | 4292 | CB | ARG | A | 534 | 2.658 | −41.558 | −20.735 | 1.00 | 32.16 | C |
| ATOM | 4293 | CG | ARG | A | 534 | 3.467 | −40.335 | −20.381 | 1.00 | 32.37 | C |
| ATOM | 4294 | CD | ARG | A | 534 | 3.306 | −39.306 | −21.469 | 1.00 | 33.07 | C |
| ATOM | 4295 | NE | ARG | A | 534 | 4.388 | −38.331 | −21.407 | 1.00 | 37.32 | N |
| ATOM | 4296 | CZ | ARG | A | 534 | 4.386 | −37.251 | −20.638 | 1.00 | 36.23 | C |
| ATOM | 4297 | NH1 | ARG | A | 534 | 3.366 | −36.987 | −19.821 | 1.00 | 38.45 | N |
| ATOM | 4298 | NH2 | ARG | A | 534 | 5.414 | −36.442 | −20.688 | 1.00 | 35.18 | N |
| ATOM | 4299 | C | ARG | A | 534 | 0.543 | −42.707 | −21.022 | 1.00 | 31.09 | C |
| ATOM | 4300 | O | ARG | A | 534 | 0.374 | −43.628 | −20.210 | 1.00 | 35.36 | O |
| ATOM | 4301 | N | TYR | A | 535 | 0.266 | −42.777 | −22.322 | 1.00 | 31.81 | N |
| ATOM | 4302 | CA | TYR | A | 535 | −0.155 | −43.982 | −23.012 | 1.00 | 33.02 | C |
| ATOM | 4303 | CB | TYR | A | 535 | −1.664 | −43.917 | −23.284 | 1.00 | 35.14 | C |
| ATOM | 4304 | CG | TYR | A | 535 | −2.262 | −45.012 | −24.172 | 1.00 | 38.46 | C |
| ATOM | 4305 | CD1 | TYR | A | 535 | −1.847 | −46.352 | −24.082 | 1.00 | 40.98 | C |
| ATOM | 4306 | CE1 | TYR | A | 535 | −2.416 | −47.347 | −24.879 | 1.00 | 38.50 | C |
| ATOM | 4307 | CZ | TYR | A | 535 | −3.410 | −47.005 | −25.779 | 1.00 | 40.96 | C |
| ATOM | 4308 | OH | TYR | A | 535 | −3.997 | −47.969 | −26.566 | 1.00 | 43.58 | O |
| ATOM | 4309 | CE2 | TYR | A | 535 | −3.839 | −45.691 | −25.887 | 1.00 | 40.74 | C |
| ATOM | 4310 | CD2 | TYR | A | 535 | −3.275 | −44.711 | −25.081 | 1.00 | 39.91 | C |
| ATOM | 4311 | C | TYR | A | 535 | 0.641 | −43.972 | −24.307 | 1.00 | 34.68 | C |
| ATOM | 4312 | O | TYR | A | 535 | 0.707 | −42.958 | −24.988 | 1.00 | 34.57 | O |
| ATOM | 4313 | N | CYS | A | 536 | 1.299 | −45.072 | −24.646 | 1.00 | 36.32 | N |
| ATOM | 4314 | CA | CYS | A | 536 | 2.040 | −45.105 | −25.915 | 1.00 | 34.23 | C |
| ATOM | 4315 | CB | CYS | A | 536 | 3.491 | −44.687 | −25.670 | 1.00 | 38.37 | C |
| ATOM | 4316 | SG | CYS | A | 536 | 4.689 | −45.411 | −26.793 | 1.00 | 48.77 | S |
| ATOM | 4317 | C | CYS | A | 536 | 1.944 | −46.467 | −26.580 | 1.00 | 31.90 | C |
| ATOM | 4318 | O | CYS | A | 536 | 1.870 | −47.475 | −25.884 | 1.00 | 34.91 | O |
| ATOM | 4319 | N | THR | A | 537 | 1.942 | −46.510 | −27.910 | 1.00 | 30.78 | N |
| ATOM | 4320 | CA | THR | A | 537 | 1.737 | −47.770 | −28.639 | 1.00 | 33.05 | C |
| ATOM | 4321 | CB | THR | A | 537 | 0.396 | −47.775 | −29.416 | 1.00 | 33.07 | C |
| ATOM | 4322 | OG1 | THR | A | 537 | −0.694 | −47.698 | −28.483 | 1.00 | 35.08 | O |
| ATOM | 4323 | CG2 | THR | A | 537 | 0.221 | −49.041 | −30.227 | 1.00 | 30.45 | C |
| ATOM | 4324 | C | THR | A | 537 | 2.897 | −48.055 | −29.571 | 1.00 | 35.74 | C |
| ATOM | 4325 | O | THR | A | 537 | 3.150 | −47.277 | −30.474 | 1.00 | 37.19 | O |
| ATOM | 4326 | N | LEU | A | 538 | 3.622 | −49.154 | −29.344 | 1.00 | 37.51 | N |
| ATOM | 4327 | CA | LEU | A | 538 | 4.808 | −49.423 | −30.161 | 1.00 | 36.86 | C |
| ATOM | 4328 | CB | LEU | A | 538 | 6.076 | −49.506 | −29.313 | 1.00 | 39.51 | C |
| ATOM | 4329 | CG | LEU | A | 538 | 6.461 | −48.231 | −28.582 | 1.00 | 38.92 | C |
| ATOM | 4330 | CD1 | LEU | A | 538 | 7.347 | −48.497 | −27.381 | 1.00 | 33.86 | C |
| ATOM | 4331 | CD2 | LEU | A | 538 | 7.143 | −47.311 | −29.564 | 1.00 | 45.41 | C |
| ATOM | 4332 | C | LEU | A | 538 | 4.673 | −50.651 | −31.037 | 1.00 | 36.75 | C |
| ATOM | 4333 | O | LEU | A | 538 | 4.164 | −51.701 | −30.609 | 1.00 | 38.98 | O |
| ATOM | 4334 | N | GLU | A | 539 | 5.144 | −50.512 | −32.269 | 1.00 | 38.21 | N |
| ATOM | 4335 | CA | GLU | A | 539 | 5.106 | −51.594 | −33.233 | 1.00 | 43.00 | C |
| ATOM | 4336 | CB | GLU | A | 539 | 4.263 | −51.207 | −34.444 | 1.00 | 46.10 | C |
| ATOM | 4337 | CG | GLU | A | 539 | 2.766 | −51.236 | −34.142 | 1.00 | 50.71 | C |
| ATOM | 4338 | CD | GLU | A | 539 | 1.916 | −50.805 | −55.315 | 1.00 | 55.28 | C |
| ATOM | 4339 | OE1 | GLU | A | 539 | 1.003 | −49.981 | −35.117 | 1.00 | 64.31 | O |
| ATOM | 4340 | OE2 | GLU | A | 539 | 2.156 | −51.284 | −36.437 | 1.00 | 51.71 | O |
| ATOM | 4341 | C | GLU | A | 539 | 6.511 | −52.033 | −33.612 | 1.00 | 42.12 | C |
| ATOM | 4342 | O | GLU | A | 539 | 7.282 | −51.278 | −34.200 | 1.00 | 45.71 | O |
| ATOM | 4343 | N | PHE | A | 540 | 6.830 | −53.258 | −33.227 | 1.00 | 39.78 | N |
| ATOM | 4344 | CA | PHE | A | 540 | 8.150 | −53.837 | −33.426 | 1.00 | 37.77 | C |
| ATOM | 4345 | CB | PHE | A | 540 | 8.627 | −54.484 | −32.130 | 1.00 | 36.04 | C |
| ATOM | 4346 | CG | PHE | A | 540 | 8.925 | −53.483 | −31.032 | 1.00 | 38.82 | C |
| ATOM | 4347 | CD1 | PHE | A | 540 | 10.083 | −52.694 | −31.072 | 1.00 | 33.19 | C |
| ATOM | 4348 | CE1 | PHE | A | 540 | 10.354 | −51.773 | −30.072 | 1.00 | 32.53 | C |
| ATOM | 4349 | CZ | PHE | A | 540 | 9.471 | −51.603 | −29.015 | 1.00 | 33.59 | C |
| ATOM | 4350 | CE2 | PHE | A | 540 | 8.293 | −52.358 | −28.974 | 1.00 | 40.86 | C |
| ATOM | 4351 | CD2 | PHE | A | 540 | 8.026 | −53.297 | −29.971 | 1.00 | 39.80 | C |
| ATOM | 4352 | C | PHE | A | 540 | 8.162 | −54.851 | −34.566 | 1.00 | 39.93 | C |
| ATOM | 4353 | O | PHE | A | 540 | 7.672 | −55.975 | −34.411 | 1.00 | 36.72 | O |
| ATOM | 4354 | N | PRO | A | 541 | 8.726 | −54.455 | −35.723 | 1.00 | 42.33 | N |
| ATOM | 4355 | CA | PRO | A | 541 | 8.883 | −55.410 | −36.821 | 1.00 | 46.33 | C |
| ATOM | 4356 | CB | PRO | A | 541 | 9.375 | −54.548 | −37.993 | 1.00 | 43.23 | C |
| ATOM | 4357 | CG | PRO | A | 541 | 9.963 | −53.334 | −37.362 | 1.00 | 45.71 | C |
| ATOM | 4358 | CD | PRO | A | 541 | 9.230 | −53.115 | −36.067 | 1.00 | 43.18 | C |
| ATOM | 4359 | C | PRO | A | 541 | 9.905 | −56.438 | −36.429 | 1.00 | 45.99 | C |
| ATOM | 4360 | O | PRO | A | 541 | 10.999 | −56.081 | −36.021 | 1.00 | 57.25 | O |
| ATOM | 4561 | N | VAL | A | 542 | 9.514 | −57.700 | −36.501 | 1.00 | 43.43 | N |
| ATOM | 4362 | CA | VAL | A | 542 | 10.353 | −58.822 | −36.124 | 1.00 | 53.35 | C |
| ATOM | 4363 | CB | VAL | A | 542 | 9.691 | −59.627 | −34.996 | 1.00 | 53.79 | C |
| ATOM | 4364 | CG1 | VAL | A | 542 | 10.407 | −60.947 | −34.740 | 1.00 | 53.82 | C |
| ATOM | 4365 | CG2 | VAL | A | 542 | 9.654 | −58.798 | −33.733 | 1.00 | 53.68 | C |
| ATOM | 4366 | C | VAL | A | 542 | 10.567 | −59.723 | −37.344 | 1.00 | 61.97 | C |
| ATOM | 4367 | O | VAL | A | 542 | 9.615 | −60.011 | −38.091 | 1.00 | 53.68 | O |
| ATOM | 4368 | N | GLU | A | 543 | 11.811 | −60.162 | −37.542 | 1.00 | 63.41 | N |
| ATOM | 4369 | CA | GLU | A | 543 | 12.162 | −60.946 | −38.719 | 1.00 | 68.50 | C |
| ATOM | 4370 | CB | GLU | A | 543 | 12.864 | −60.976 | −39.779 | 100 | 74.54 | C |
| ATOM | 4371 | CG | GLU | A | 543 | 14.227 | −59.529 | −39.380 | 1.00 | 86.41 | C |
| ATOM | 4372 | CD | GLU | A | 543 | 14.129 | −58.281 | −38.528 | 1.00 | 103.86 | C |
| ATOM | 4373 | OE1 | GLU | A | 543 | 13.326 | −57.182 | −38.876 | 1.00 | 112.71 | O |
| ATOM | 4374 | OE2 | GLU | A | 543 | 14.857 | −58.199 | −37.509 | 1.00 | 108.89 | O |
| ATOM | 4375 | C | GLU | A | 543 | 12.924 | −62.260 | −38.450 | 1.00 | 64.74 | C |
| ATOM | 4376 | O | GLU | A | 543 | 13.679 | −62.725 | −39.302 | 1.00 | 67.11 | O |
| ATOM | 4377 | N | VAL | A | 544 | 12.724 | −62.853 | −37.278 | 1.00 | 55.79 | N |
| ATOM | 4378 | CA | VAL | A | 544 | 13.039 | −64.268 | −37.089 | 1.00 | 58.41 | C |
| ATOM | 4379 | CB | VAL | A | 544 | 14.307 | −64.531 | −36.236 | 1.00 | 64.35 | C |
| ATOM | 4380 | CG1 | VAL | A | 544 | 15.525 | −63.825 | −36.820 | 1.00 | 78.92 | C |
| ATOM | 4381 | CG2 | VAL | A | 544 | 14.107 | −64.145 | −34.775 | 1.00 | 64.22 | C |
| ATOM | 4382 | C | VAL | A | 544 | 11.850 | −64.895 | −36.395 | 1.00 | 54.74 | C |
| ATOM | 4383 | O | VAL | A | 544 | 10.961 | −64.187 | −35.974 | 1.00 | 58.25 | O |
| ATOM | 4384 | N | ASN | A | 545 | 11.826 | −66.112 | −36.271 | 1.00 | 54.18 | N |
| ATOM | 4385 | CA | ASN | A | 545 | 10.847 | −66.141 | −35.414 | 1.00 | 55.71 | C |
| ATOM | 4386 | CB | ASN | A | 545 | 10.521 | −68.264 | −35.875 | 1.00 | 56.96 | C |
| ATOM | 4387 | CB | ASN | A | 545 | 10.240 | −68.344 | −37.361 | 1.00 | 60.84 | C |
| ATOM | 4388 | OD1 | ASN | A | 545 | 9.243 | −67.803 | −37.857 | 1.00 | 60.63 | O |
| ATOM | 4389 | ND2 | ASN | A | 545 | 11.128 | −69.009 | −38.085 | 1.00 | 56.88 | N |
| ATOM | 4390 | C | ASN | A | 545 | 11.475 | −66.854 | −34.048 | 1.00 | 55.79 | C |
| ATOM | 4391 | O | ASN | A | 545 | 12.704 | −66.828 | −33.945 | 1.00 | 57.77 | O |
| ATOM | 4392 | N | THR | A | 546 | 10.643 | −66.912 | −33.009 | 1.00 | 51.07 | N |
| ATOM | 4393 | CA | THR | A | 546 | 11.110 | −66.808 | −31.628 | 1.00 | 47.27 | C |
| ATOM | 4394 | CB | THR | A | 546 | 12.009 | −65.569 | −31.385 | 1.00 | 44.80 | C |
| ATOM | 4395 | OG1 | THR | A | 546 | 12.421 | −65.565 | −30.018 | 1.00 | 49.88 | O |
| ATOM | 4396 | CG2 | THR | A | 546 | 11.304 | −64.272 | −31.702 | 1.00 | 41.22 | C |
| ATOM | 4397 | C | THR | A | 546 | 9.982 | −66.815 | −30.612 | 1.00 | 47.57 | C |
| ATOM | 4398 | O | THR | A | 546 | 8.813 | −67.034 | −30.959 | 1.00 | 52.26 | O |
| ATOM | 4399 | N | VAL | A | 547 | 10.354 | −66.591 | −29.355 | 1.00 | 44.96 | N |
| ATOM | 4400 | CA | VAL | A | 547 | 9.403 | −66.430 | −28.266 | 1.00 | 44.34 | C |
| ATOM | 4401 | CB | VAL | A | 547 | 9.454 | −67.635 | −27.354 | 1.00 | 46.29 | C |
| ATOM | 4402 | CG1 | VAL | A | 547 | 8.315 | −67.590 | −26.347 | 1.00 | 42.97 | C |
| ATOM | 4403 | CG2 | VAL | A | 547 | 9.407 | −68.894 | −28.198 | 1.00 | 49.35 | C |
| ATOM | 4404 | C | VAL | A | 547 | 9.686 | −65.191 | −27.422 | 1.00 | 45.25 | C |
| ATOM | 4405 | O | VAL | A | 547 | 10.769 | −65.059 | −26.859 | 1.00 | 48.33 | O |
| ATOM | 4406 | N | LEU | A | 548 | 8.699 | −64.298 | −27.332 | 1.00 | 43.62 | N |
| ATOM | 4407 | CA | LEU | A | 548 | 8.788 | −63.079 | −26.532 | 1.00 | 38.43 | C |
| ATOM | 4408 | CB | LEU | A | 548 | 7.690 | −62.124 | −26.938 | 1.00 | 37.12 | C |
| ATOM | 4409 | CG | LEU | A | 548 | 7.549 | −60.141 | −26.320 | 1.00 | 35.06 | C |
| ATOM | 4410 | CD1 | LEU | A | 548 | 8.633 | −59.814 | −26.852 | 1.00 | 33.20 | C |
| ATOM | 4411 | CD2 | LEU | A | 548 | 6.161 | −60.217 | −26.711 | 1.00 | 31.26 | C |
| ATOM | 4412 | C | LEU | A | 518 | 8.637 | −63.590 | −25.075 | 1.00 | 38.09 | C |
| ATOM | 4413 | O | LEU | A | 548 | 7.696 | −64.075 | −24.684 | 1.00 | 41.95 | O |
| ATOM | 4414 | N | HIS | A | 549 | 9.571 | −62.888 | −24.273 | 1.00 | 35.37 | N |
| ATOM | 4415 | CA | HIS | A | 549 | 9.572 | −63.121 | −22.829 | 1.00 | 33.02 | C |
| ATOM | 4416 | CB | HIS | A | 549 | 10.858 | −63.868 | −22.414 | 1.00 | 34.31 | C |
| ATOM | 4417 | CG | HIS | A | 549 | 10.945 | −65.263 | −22.946 | 1.00 | 36.97 | C |
| ATOM | 4418 | ND1 | HIS | A | 549 | 11.360 | −65.548 | −24.230 | 1.00 | 39.69 | N |
| ATOM | 4419 | CE1 | HIS | A | 549 | 11.319 | −66.854 | −24.418 | 1.00 | 38.88 | C |
| ATOM | 4420 | NE2 | HIS | A | 549 | 10.915 | −67.427 | −23.295 | 1.00 | 34.95 | N |
| ATOM | 4421 | CD2 | HIS | A | 549 | 10.672 | −66.455 | −22.363 | 1.00 | 36.43 | C |
| ATOM | 4422 | C | HIS | A | 549 | 9.519 | −61.802 | −22.067 | 1.00 | 29.26 | C |
| ATOM | 4423 | O | HIS | A | 549 | 9.292 | −61.285 | −20.851 | 1.00 | 27.95 | O |
| ATOM | 4424 | N | GLY | A | 550 | 9.798 | −60.703 | −22.755 | 1.00 | 27.90 | N |
| ATOM | 4425 | CA | GLY | A | 550 | 9.808 | −59.413 | −22.079 | 1.00 | 29.27 | C |
| ATOM | 4426 | C | GLY | A | 550 | 10.290 | −58.264 | −22.917 | 1.00 | 27.71 | C |
| ATOM | 4427 | O | GLY | A | 550 | 10.519 | −58.412 | −24.114 | 1.00 | 30.25 | O |
| ATOM | 4428 | N | PHE | A | 551 | 10.417 | −57.115 | −22.271 | 1.00 | 26.66 | N |
| ATOM | 4429 | CA | PHE | A | 551 | 10.863 | −55.881 | −22.892 | 1.00 | 28.76 | C |
| ATOM | 4430 | CB | PHE | A | 551 | 9.692 | −54.888 | −23.014 | 1.00 | 29.94 | C |
| ATOM | 4431 | CG | PHE | A | 551 | 8.657 | −55.342 | −23.986 | 1.00 | 29.55 | C |
| ATOM | 4432 | CD1 | PHE | A | 551 | 7.708 | −56.283 | −23.619 | 1.00 | 29.41 | C |
| ATOM | 4433 | CE1 | PHE | A | 551 | 6.788 | −56.755 | −24.544 | 1.00 | 30.09 | C |
| ATOM | 4434 | CZ | PHE | A | 551 | 6.824 | −56.309 | −25.853 | 1.00 | 30.37 | C |
| ATOM | 4435 | CE2 | PHE | A | 551 | 7.767 | −55.372 | −26.243 | 1.00 | 31.20 | C |
| ATOM | 4436 | CD2 | PHE | A | 551 | 8.697 | −54.909 | −25.309 | 1.00 | 32.06 | C |
| ATOM | 4437 | C | PHE | A | 551 | 11.983 | −55.299 | −22.065 | 1.00 | 32.30 | C |
| ATOM | 4438 | O | PHE | A | 551 | 11.807 | −55.035 | −20.866 | 1.00 | 31.63 | O |
| ATOM | 4439 | N | ALA | A | 552 | 13.161 | −55.191 | −22.698 | 1.00 | 33.75 | N |
| ATOM | 4440 | CA | ALA | A | 552 | 14.257 | −54.363 | −22.201 | 1.00 | 30.36 | C |
| ATOM | 4441 | CB | ALA | A | 552 | 15.518 | −54.610 | −23.022 | 1.00 | 31.49 | C |
| ATOM | 4442 | C | ALA | A | 552 | 13.869 | −52.894 | −22.295 | 1.00 | 28.76 | C |
| ATOM | 4443 | O | ALA | A | 552 | 13.383 | −52.449 | −23.345 | 1.00 | 28.28 | O |
| ATOM | 4444 | N | GLY | A | 553 | 14.056 | −52.146 | −21.209 | 1.00 | 26.19 | N |
| ATOM | 4445 | CA | GLY | A | 553 | 13.997 | −50.684 | −21.303 | 1.00 | 28.08 | C |
| ATOM | 4146 | C | GLY | A | 553 | 15.374 | −50.020 | −21.132 | 1.00 | 31.88 | C |
| ATOM | 4447 | O | GLY | A | 553 | 16.289 | −50.565 | −20.469 | 1.00 | 31.48 | O |
| ATOM | 4448 | N | TYR | A | 554 | 15.526 | −48.850 | −21.748 | 1.00 | 31.52 | N |
| ATOM | 4449 | CA | TYR | A | 554 | 16.740 | −48.058 | −21.645 | 1.00 | 31.98 | C |
| ATOM | 4450 | CB | TYR | A | 554 | 17.653 | −48.272 | −22.859 | 1.00 | 30.24 | C |
| ATOM | 4451 | CG | TYR | A | 554 | 17.892 | −49.720 | −23.310 | 1.00 | 28.60 | C |
| ATOM | 4452 | CD1 | TYR | A | 554 | 17.138 | −50.288 | −24.341 | 1.00 | 26.94 | C |
| ATOM | 4453 | CE1 | TYR | A | 554 | 17.369 | −51.587 | −24.762 | 1.00 | 26.39 | C |
| ATOM | 4454 | CZ | TYR | A | 554 | 18.383 | −52.335 | −24.157 | 1.00 | 26.08 | C |
| ATOM | 4455 | OH | TYR | A | 554 | 18.623 | −53.637 | −24.569 | 1.00 | 26.89 | O |
| ATOM | 4456 | CE2 | TYR | A | 554 | 19.128 | −51.799 | −23.134 | 1.00 | 23.81 | C |
| ATOM | 4457 | CD2 | TYR | A | 554 | 18.895 | −50.499 | −22.729 | 1.00 | 26.36 | C |
| ATOM | 4458 | C | TYR | A | 554 | 16.284 | −46.614 | −21.641 | 1.00 | 34.81 | C |
| ATOM | 4459 | O | TYR | A | 554 | 15.076 | −46.329 | −21.724 | 1.00 | 33.36 | O |
| ATOM | 4460 | N | PHE | A | 555 | 17.228 | −45.687 | −21.575 | 1.00 | 34.41 | N |
| ATOM | 4461 | CA | PHE | A | 555 | 16.846 | −44.288 | −21.652 | 1.00 | 36.58 | C |
| ATOM | 4462 | CB | PHE | A | 555 | 16.511 | −43.755 | −20.252 | 1.00 | 39.29 | C |
| ATOM | 4463 | CG | PHE | A | 555 | 17.727 | −43.463 | −19.398 | 1.00 | 38.20 | C |
| ATOM | 4464 | CD1 | PHE | A | 555 | 18.209 | −44.440 | −18.584 | 1.00 | 38.56 | C |
| ATOM | 4465 | CE1 | PHE | A | 555 | 19.381 | −44.163 | −17.795 | 1.00 | 39.54 | C |
| ATOM | 4466 | CZ | PHE | A | 555 | 19.963 | −42.899 | −17.818 | 1.00 | 38.33 | C |
| ATOM | 4467 | CE2 | PHE | A | 555 | 19.432 | −41.912 | −18.629 | 1.00 | 37.28 | C |
| ATOM | 4468 | CD2 | PHE | A | 555 | 18.322 | −42.195 | −19.412 | 1.00 | 37.86 | C |
| ATOM | 4469 | C | PHE | A | 555 | 17.938 | −43.425 | −22.259 | 1.00 | 37.99 | C |
| ATOM | 4470 | O | PHE | A | 555 | 19.111 | −43.817 | −22.287 | 1.00 | 39.68 | O |
| ATOM | 4471 | N | GLU | A | 556 | 17.543 | −42.226 | −22.678 | 1.00 | 38.18 | N |
| ATOM | 4472 | CA | GLU | A | 556 | 18.468 | −41.171 | −23.065 | 1.00 | 41.35 | C |
| ATOM | 4473 | CB | GLU | A | 556 | 18.494 | −40.978 | −24.589 | 1.00 | 40.73 | C |
| ATOM | 4474 | CG | GLU | A | 556 | 19.267 | −42.039 | −25.376 | 1.00 | 43.14 | C |
| ATOM | 4475 | CD | GLU | A | 556 | 19.159 | −41.846 | −26.888 | 1.00 | 44.55 | C |
| ATOM | 4476 | OE1 | GLU | A | 556 | 18.370 | −40.982 | −27.345 | 1.00 | 43.88 | O |
| ATOM | 4477 | OE2 | GLU | A | 556 | 19.862 | −42.561 | −27.627 | 1.00 | 42.69 | O |
| ATOM | 4478 | C | GLU | A | 556 | 18.029 | −39.881 | −22.392 | 1.00 | 43.30 | C |
| ATOM | 4479 | O | GLU | A | 556 | 16.825 | −39.679 | −22.128 | 1.00 | 47.79 | O |
| ATOM | 4480 | N | THR | A | 557 | 19.000 | −39.024 | −22.094 | 1.00 | 39.66 | N |
| ATOM | 4481 | CA | THR | A | 557 | 18.708 | −37.717 | −21.527 | 1.00 | 42.85 | C |
| ATOM | 4482 | CB | THR | A | 557 | 18.714 | −37.704 | −19.970 | 1.00 | 43.11 | C |
| ATOM | 4483 | OG1 | THR | A | 557 | 18.091 | −36.494 | −19.498 | 1.00 | 48.44 | O |
| ATOM | 4484 | CG1 | THR | A | 557 | 20.108 | −37.797 | −19.398 | 1.00 | 36.20 | C |
| ATOM | 4485 | C | THR | A | 557 | 19.646 | −36.636 | −22.062 | 1.00 | 42.03 | C |
| ATOM | 4486 | O | THR | A | 557 | 20.843 | −36.862 | −22.241 | 1.00 | 40.52 | O |
| ATOM | 4487 | N | VAL | A | 558 | 19.066 | −35.473 | −22.327 | 1.00 | 40.06 | N |
| ATOM | 4488 | CA | VAL | A | 558 | 19.820 | −34.284 | −22.656 | 1.00 | 38.02 | C |
| ATOM | 4489 | CB | VAL | A | 558 | 19.055 | −33.371 | −23.639 | 1.00 | 36.28 | C |
| ATOM | 4490 | CG1 | VAL | A | 558 | 19.762 | −32.017 | −23.815 | 1.00 | 34.87 | C |
| ATOM | 4491 | CG2 | VAL | A | 558 | 18.872 | −34.071 | −24.984 | 1.00 | 32.39 | C |
| ATOM | 4492 | C | VAL | A | 558 | 20.060 | −33.574 | −21.343 | 1.00 | 38.11 | C |
| ATOM | 4493 | O | VAL | A | 558 | 19.129 | −33.031 | −20.720 | 1.00 | 38.03 | O |
| ATOM | 4494 | N | LEU | A | 559 | 21.309 | −33.627 | −20.913 | 1.00 | 36.93 | N |
| ATOM | 4495 | CA | LEU | A | 559 | 21.780 | −32.861 | −19.767 | 1.00 | 42.23 | C |
| ATOM | 4496 | CB | LEU | A | 559 | 23.203 | −33.315 | −19.405 | 1.00 | 41.25 | C |
| ATOM | 4497 | CG | LEU | A | 559 | 23.479 | −34.203 | −18.190 | 1.00 | 41.81 | C |
| ATOM | 4498 | CD1 | LEU | A | 559 | 22.328 | −35.140 | −17.883 | 1.00 | 37.30 | C |
| ATOM | 4499 | CD2 | LEU | A | 559 | 24.818 | −34.949 | −18.308 | 1.00 | 39.67 | C |
| ATOM | 4500 | C | LEU | A | 559 | 21.747 | −31.363 | −20.112 | 1.00 | 43.20 | C |
| ATOM | 4501 | O | LEU | A | 559 | 21.018 | −30.588 | −19.491 | 1.00 | 45.06 | O |
| ATOM | 4502 | N | TYR | A | 560 | 22.523 | −30.981 | −21.131 | 1.00 | 47.55 | N |
| ATOM | 4503 | CA | TYR | A | 560 | 22.526 | −29.516 | −21.697 | 1.00 | 46.76 | C |
| ATOM | 4504 | CB | TYR | A | 560 | 23.362 | −28.555 | −20.840 | 1.00 | 45.49 | C |
| ATOM | 4505 | CG | TYR | A | 560 | 23.200 | −27.200 | −21.237 | 1.00 | 48.95 | C |
| ATOM | 4506 | CD1 | TYR | A | 560 | 21.983 | −26.531 | −21.041 | 1.00 | 45.72 | C |
| ATOM | 4507 | CE1 | TYR | A | 560 | 21.836 | −25.202 | −21.409 | 1.00 | 49.54 | C |
| ATOM | 4508 | CZ | TYR | A | 560 | 22.920 | −24.514 | −21.984 | 1.00 | 50.74 | C |
| ATOM | 4509 | OH | TYR | A | 560 | 22.769 | −23.192 | −22.355 | 1.00 | 47.80 | O |
| ATOM | 4510 | CE2 | TYR | A | 560 | 24.129 | −23.158 | −22.195 | 1.00 | 45.64 | C |
| ATOM | 4511 | CD2 | TYR | A | 560 | 24.264 | −26.489 | −21.824 | 1.00 | 48.64 | C |
| ATOM | 4512 | C | TYR | A | 560 | 23.014 | −29.588 | −23.155 | 1.00 | 47.86 | C |
| ATOM | 4513 | O | TYR | A | 560 | 24.166 | −29.956 | −23.463 | 1.00 | 45.71 | O |
| ATOM | 4514 | N | GLN | A | 561 | 22.125 | −29.163 | −24.048 | 1.00 | 47.64 | N |
| ATOM | 4515 | CA | GLN | A | 561 | 22.453 | −29.041 | −25.469 | 1.00 | 49.14 | C |
| ATOM | 4516 | CB | GLN | A | 561 | 23.502 | −27.936 | −25.698 | 1.00 | 47.70 | C |
| ATOM | 4517 | CG | GLN | A | 561 | 22.873 | −26.551 | −25.652 | 1.00 | 47.84 | C |
| ATOM | 4518 | CD | GLN | A | 561 | 23.873 | −25.403 | −25.636 | 1.00 | 50.24 | C |
| ATOM | 4519 | OE1 | GLN | A | 561 | 25.104 | −25.592 | −25.745 | 1.00 | 45.33 | O |
| ATOM | 4520 | NE2 | GLN | A | 561 | 23.339 | −24.188 | −25.506 | 1.00 | 45.55 | N |
| ATOM | 4521 | C | GLN | A | 561 | 22.891 | −30.358 | −26.101 | 1.00 | 48.85 | C |
| ATOM | 4522 | O | GLN | A | 561 | 22.055 | −31.222 | −26.346 | 1.00 | 48.76 | O |
| ATOM | 4523 | N | ASP | A | 562 | 24.188 | −30.492 | −26.375 | 1.00 | 46.70 | N |
| ATOM | 4524 | CA | ASP | A | 562 | 24.725 | −31.697 | −27.002 | 1.00 | 45.21 | C |
| ATOM | 4525 | CB | ASP | A | 562 | 25.766 | −31.365 | −28.091 | 1.00 | 46.34 | C |
| ATOM | 4526 | CG | ASP | A | 562 | 27.030 | −30.695 | −27.538 | 1.00 | 49.44 | C |
| ATOM | 4327 | OD1 | ASP | A | 562 | 26.915 | −29.642 | −26.859 | 1.00 | 48.63 | O |
| ATOM | 4528 | OD2 | ASP | A | 562 | 28.142 | −31.216 | −27.805 | 1.00 | 51.94 | O |
| ATOM | 4329 | C | ASP | A | 562 | 25.327 | −32.635 | −25.986 | 1.00 | 43.64 | C |
| ATOM | 4530 | O | ASP | A | 562 | 25.801 | −33.718 | −26.332 | 1.00 | 45.33 | O |
| ATOM | 4331 | N | ILE | A | 563 | 25.322 | −32.226 | −24.730 | 1.00 | 42.89 | N |
| ATOM | 4532 | CA | ILE | A | 563 | 25.885 | −33.079 | −23.713 | 1.00 | 46.30 | C |
| ATOM | 4533 | CB | ILE | A | 563 | 26.540 | −32.278 | −22.575 | 1.00 | 48.26 | C |
| ATOM | 4534 | CG1 | ILE | A | 563 | 27.893 | −31.737 | −23.043 | 1.00 | 50.44 | C |
| ATOM | 4535 | CD1 | ILE | A | 563 | 27.793 | −30.647 | −24.077 | 1.00 | 54.09 | C |
| ATOM | 4536 | CG2 | ILE | A | 563 | 26.798 | −33.178 | −21.366 | 1.00 | 47.82 | C |
| ATOM | 4537 | C | ILE | A | 563 | 24.804 | −34.016 | −23.206 | 1.00 | 45.57 | C |
| ATOM | 4538 | O | ILE | A | 563 | 23.784 | −33.565 | −22.670 | 1.00 | 46.14 | O |
| ATOM | 4539 | N | THR | A | 564 | 25.033 | −35.315 | −23.401 | 1.00 | 43.66 | N |
| ATOM | 4540 | CA | THR | A | 564 | 24.024 | −36.329 | −23.103 | 1.00 | 43.08 | C |
| ATOM | 4541 | CB | THR | A | 564 | 23.422 | −36.952 | −24.379 | 1.00 | 43.34 | C |
| ATOM | 4542 | OG1 | THR | A | 564 | 24.461 | −37.592 | −25.129 | 1.00 | 41.15 | O |
| ATOM | 4543 | CG2 | THR | A | 564 | 22.673 | −35.900 | −25.227 | 1.00 | 38.71 | C |
| ATOM | 4544 | C | THR | A | 564 | 24.544 | −37.483 | −22.277 | 1.00 | 42.38 | C |
| ATOM | 4545 | O | THR | A | 564 | 25.754 | −37.686 | −22.111 | 1.00 | 44.36 | O |
| ATOM | 4546 | N | LEU | A | 565 | 23.594 | −33.228 | −21.743 | 1.00 | 40.72 | N |
| ATOM | 4547 | CA | LEU | A | 565 | 23.860 | −39.530 | −21.147 | 1.00 | 38.68 | C |
| ATOM | 4548 | CB | LEU | A | 565 | 23.611 | −39.493 | −19.642 | 1.00 | 39.17 | C |
| ATOM | 4549 | CG | LEU | A | 565 | 24.482 | −38.595 | −18.751 | 1.00 | 40.74 | C |
| ATOM | 4550 | CD1 | LEU | A | 565 | 23.953 | −38.673 | −17.330 | 1.00 | 38.87 | C |
| ATOM | 4551 | CD2 | LEU | A | 565 | 25.972 | −38.966 | −18.798 | 1.00 | 38.06 | C |
| ATOM | 4552 | C | LEU | A | 565 | 22.910 | −40.528 | −21.795 | 1.00 | 36.91 | C |
| ATOM | 4553 | O | LEU | A | 565 | 21.722 | −40.202 | −22.072 | 1.00 | 35.07 | O |
| ATOM | 4554 | N | SER | A | 566 | 23.420 | −41.740 | −22.013 | 1.00 | 34.32 | N |
| ATOM | 4555 | CA | SER | A | 566 | 22.630 | −42.784 | −22.651 | 1.00 | 32.79 | C |
| ATOM | 4556 | CB | SER | A | 566 | 22.659 | −42.596 | −24.160 | 1.00 | 30.65 | C |
| ATOM | 4557 | OG | SER | A | 566 | 22.177 | −43.742 | −24.802 | 1.00 | 35.55 | O |
| ATOM | 4558 | C | SER | A | 566 | 23.045 | −44.200 | −22.250 | 1.00 | 34.88 | C |
| ATOM | 4559 | O | SER | A | 566 | 24.243 | −44.484 | −22.091 | 1.00 | 34.26 | O |
| ATOM | 4560 | N | ILE | A | 567 | 22.042 | −45.060 | −22.041 | 1.00 | 34.36 | N |
| ATOM | 4561 | CA | ILE | A | 567 | 22.251 | −46.513 | −21.941 | 1.00 | 34.98 | C |
| ATOM | 4562 | CB | ILE | A | 567 | 21.794 | −47.138 | −20.604 | 1.00 | 36.17 | C |
| ATOM | 4563 | CG1 | ILE | A | 567 | 20.283 | −46.955 | −20.378 | 1.00 | 35.09 | C |
| ATOM | 4564 | CD1 | ILE | A | 567 | 19.772 | −47.857 | −19.272 | 1.00 | 34.93 | C |
| ATOM | 4565 | CG2 | ILE | A | 567 | 22.646 | −46.642 | −19.447 | 1.00 | 34.25 | C |
| ATOM | 4566 | C | ILE | A | 567 | 21.542 | −47.274 | −23.049 | 1.00 | 35.98 | C |
| ATOM | 4567 | O | ILE | A | 567 | 21.458 | −48.497 | −23.010 | 1.00 | 37.22 | O |
| ATOM | 4568 | N | ARG | A | 568 | 21.026 | −46.553 | −24.035 | 1.00 | 37.18 | N |
| ATOM | 4569 | CA | ARG | A | 568 | 20.475 | −47.203 | −25.200 | 1.00 | 38.53 | C |
| ATOM | 4570 | CB | ARG | A | 568 | 19.726 | −46.180 | −26.013 | 1.00 | 35.61 | C |
| ATOM | 4571 | CG | ARG | A | 568 | 19.430 | −46.624 | −27.421 | 1.00 | 37.27 | C |
| ATOM | 4572 | CD | ARG | A | 568 | 18.711 | −45.506 | −28.140 | 1.00 | 41.12 | C |
| ATOM | 4573 | NE | ARG | A | 568 | 18.468 | −45.805 | −29.544 | 1.00 | 45.59 | N |
| ATOM | 4574 | CZ | ARG | A | 568 | 18.447 | −44.873 | −30.496 | 1.00 | 49.11 | C |
| ATOM | 4575 | NH1 | ARG | A | 568 | 18.671 | −43.585 | −30.198 | 1.00 | 44.43 | N |
| ATOM | 4576 | NH2 | ARG | A | 568 | 18.218 | −45.229 | −31.752 | 1.00 | 46.98 | N |
| ATOM | 4577 | C | ARG | A | 568 | 21.651 | −47.731 | −25.995 | 1.00 | 39.69 | C |
| ATOM | 4578 | O | ARG | A | 568 | 22.467 | −46.946 | −26.428 | 1.00 | 43.37 | O |
| ATOM | 4579 | N | PRO | A | 569 | 21.726 | −49.054 | −26.218 | 1.00 | 43.58 | N |
| ATOM | 4580 | CA | PRO | A | 569 | 22.932 | −49.639 | −26.828 | 1.00 | 42.10 | C |
| ATOM | 4581 | CB | PRO | A | 569 | 22.495 | −51.072 | −27.182 | 1.00 | 41.35 | C |
| ATOM | 4582 | CG | PRO | A | 569 | 21.363 | −51.367 | −26.233 | 1.00 | 42.01 | C |
| ATOM | 4583 | CD | PRO | A | 569 | 20.636 | −50.052 | −26.098 | 1.00 | 44.34 | C |
| ATOM | 4584 | C | PRO | A | 569 | 23.465 | −48.886 | −28.059 | 1.00 | 43.79 | C |
| ATOM | 4585 | O | PRO | A | 569 | 24.651 | −48.657 | −28.125 | 1.00 | 42.51 | O |
| ATOM | 4586 | N | GLU | A | 570 | 22.612 | −48.458 | −28.993 | 1.00 | 46.30 | N |
| ATOM | 4587 | CA | GLU | A | 570 | 23.108 | −47.773 | −30.208 | 1.00 | 45.11 | C |
| ATOM | 4588 | CB | GLU | A | 570 | 21.976 | −47.484 | −31.210 | 1.00 | 46.56 | C |
| ATOM | 4589 | CG | GLU | A | 570 | 21.142 | −48.682 | −31.657 | 1.00 | 53.42 | C |
| ATOM | 4590 | CD | GLU | A | 570 | 19.919 | −48.917 | −30.738 | 1.00 | 59.56 | C |
| ATOM | 4591 | OE1 | GLU | A | 570 | 20.030 | −49.830 | −29.889 | 1.00 | 58.49 | O |
| ATOM | 4592 | OE2 | GLU | A | 570 | 18.941 | −48.167 | −30.834 | 1.00 | 64.19 | O |
| ATOM | 4593 | C | GLU | A | 570 | 23.904 | −46.469 | −29.946 | 1.00 | 44.45 | C |
| ATOM | 4594 | O | GLU | A | 570 | 24.590 | −45.970 | −30.835 | 1.00 | 45.25 | O |
| ATOM | 4595 | N | THR | A | 571 | 23.792 | −45.906 | −28.746 | 1.00 | 43.65 | N |
| ATOM | 4596 | CA | THR | A | 571 | 24.332 | −44.578 | −28.433 | 1.00 | 37.49 | C |
| ATOM | 4597 | CB | THR | A | 571 | 23.268 | −43.460 | −28.583 | 1.00 | 39.97 | C |
| ATOM | 4598 | OG1 | THR | A | 571 | 22.289 | −43.611 | −27.553 | 1.00 | 40.55 | O |
| ATOM | 4599 | CG2 | THR | A | 571 | 22.555 | −43.486 | −29.941 | 1.00 | 39.05 | C |
| ATOM | 4600 | C | THR | A | 571 | 24.790 | −44.575 | −26.983 | 1.00 | 35.77 | C |
| ATOM | 4601 | O | THR | A | 571 | 24.821 | −43.524 | −26.346 | 1.00 | 34.89 | O |
| ATOM | 4602 | N | HIS | A | 572 | 25.113 | −45.758 | −26.458 | 1.00 | 39.75 | N |
| ATOM | 4603 | CA | HIS | A | 572 | 25.543 | −45.926 | −25.060 | 1.00 | 45.87 | C |
| ATOM | 4604 | CB | HIS | A | 572 | 25.985 | −47.369 | −24.780 | 1.00 | 45.83 | C |
| ATOM | 4605 | CG | HIS | A | 572 | 25.833 | −47.781 | −23.343 | 1.00 | 50.03 | C |
| ATOM | 4606 | ND1 | HIS | A | 572 | 26.553 | −47.202 | −22.318 | 1.00 | 48.55 | N |
| ATOM | 4607 | CE1 | HIS | A | 572 | 26.208 | −47.755 | −23.169 | 1.00 | 47.09 | C |
| ATOM | 4608 | NE2 | HIS | A | 572 | 25.288 | −48.672 | −21.407 | 1.00 | 49.45 | N |
| ATOM | 4609 | CD2 | HIS | A | 572 | 25.031 | −48.707 | −22.758 | 1.00 | 50.13 | C |
| ATOM | 4610 | C | HIS | A | 572 | 26.707 | −44.975 | −24.778 | 1.00 | 45.89 | C |
| ATOM | 4611 | O | HIS | A | 572 | 27.553 | −44.795 | −25.646 | 1.00 | 45.59 | O |
| ATOM | 4612 | N | SER | A | 573 | 26.715 | −44.343 | −23.599 | 1.00 | 45.41 | N |
| ATOM | 4613 | CA | SER | A | 573 | 27.828 | −43.498 | −23.168 | 1.00 | 44.45 | C |
| ATOM | 4614 | CB | SER | A | 573 | 27.446 | −42.639 | −21.969 | 1.00 | 41.77 | C |
| ATOM | 4615 | OG | SER | A | 573 | 26.302 | −41.847 | −22.265 | 1.00 | 43.73 | O |
| ATOM | 4616 | C | SER | A | 573 | 28.973 | −44.421 | −22.812 | 1.00 | 43.40 | C |
| ATOM | 4617 | O | SER | A | 573 | 28.800 | −45.340 | −22.016 | 1.00 | 43.42 | O |
| ATOM | 4618 | N | PRO | A | 574 | 30.144 | −44.218 | −23.435 | 1.00 | 52.03 | N |
| ATOM | 4619 | CA | PRO | A | 574 | 31.212 | −45.204 | −23.198 | 1.00 | 54.00 | C |
| ATOM | 4620 | CB | PRO | A | 574 | 32.291 | −44.782 | −24.189 | 1.00 | 49.99 | C |
| ATOM | 4621 | CG | PRO | A | 574 | 31.494 | −44.164 | −25.312 | 1.00 | 53.46 | C |
| ATOM | 4622 | CD | PRO | A | 574 | 30.451 | −43.352 | −24.588 | 1.00 | 49.96 | C |
| ATOM | 4623 | C | PRO | A | 574 | 31.720 | −45.161 | −21.762 | 1.00 | 53.25 | C |
| ATOM | 4624 | O | PRO | A | 574 | 31.881 | −44.077 | −21.200 | 1.00 | 57.19 | O |
| ATOM | 4625 | N | GLY | A | 575 | 31.914 | −46.333 | −21.164 | 1.00 | 48.34 | N |
| ATOM | 4626 | CA | GLY | A | 575 | 32.419 | −46.418 | −19.798 | 1.00 | 47.94 | C |
| ATOM | 4627 | C | GLY | A | 575 | 31.420 | −46.208 | −18.667 | 1.00 | 48.79 | C |
| ATOM | 4628 | O | GLY | A | 575 | 31.728 | −46.505 | −17.514 | 1.00 | 49.08 | O |
| ATOM | 4629 | N | MET | A | 576 | 30.231 | −45.692 | −18.975 | 1.00 | 46.94 | N |
| ATOM | 4630 | CA | MET | A | 576 | 29.236 | −45.422 | −17.943 | 1.00 | 43.02 | C |
| ATOM | 4631 | CB | MET | A | 576 | 28.172 | −44.469 | −18.455 | 1.00 | 39.85 | C |
| ATOM | 4632 | CG | MET | A | 576 | 27.336 | −43.894 | −17.342 | 1.00 | 39.12 | C |
| ATOM | 4633 | SD | MET | A | 576 | 26.052 | −42.827 | −17.997 | 1.00 | 42.80 | S |
| ATOM | 4634 | CE | MET | A | 576 | 29.177 | −43.979 | −19.043 | 1.00 | 41.08 | C |
| ATOM | 4635 | C | MET | A | 576 | 28.576 | −46.702 | −17.468 | 1.00 | 43.38 | C |
| ATOM | 4636 | O | MET | A | 576 | 27.873 | −47.358 | −18.227 | 1.00 | 49.91 | O |
| ATOM | 4637 | N | PHE | A | 577 | 28.803 | −47.048 | −16.210 | 1.00 | 43.02 | N |
| ATOM | 4638 | CA | PHE | A | 577 | 28.258 | −48.273 | −15.637 | 1.00 | 42.04 | C |
| ATOM | 4639 | CB | PHE | A | 577 | 29.374 | −49.090 | −14.959 | 1.00 | 45.88 | C |
| ATOM | 4640 | CG | PHE | A | 577 | 30.106 | −48.356 | −13.858 | 1.00 | 51.74 | C |
| ATOM | 4641 | CD1 | PHE | A | 577 | 29.572 | −48.285 | −12.564 | 1.00 | 53.16 | C |
| ATOM | 4642 | CE1 | PHE | A | 577 | 30.258 | −47.622 | −11.545 | 1.00 | 54.32 | C |
| ATOM | 4643 | CZ | PHE | A | 577 | 31.487 | −47.022 | −11.809 | 1.00 | 48.76 | C |
| ATOM | 4644 | CE2 | PHE | A | 577 | 32.029 | −47.085 | −13.090 | 1.00 | 48.17 | C |
| ATOM | 4645 | CD2 | PHE | A | 577 | 31.347 | −47.747 | −14.104 | 1.00 | 49.18 | C |
| ATOM | 4646 | C | PHE | A | 577 | 27.103 | −48.039 | −14.663 | 1.00 | 37.84 | C |
| ATOM | 4647 | O | PHE | A | 577 | 26.583 | −48.977 | −14.097 | 1.00 | 41.49 | O |
| ATOM | 4648 | N | SER | A | 578 | 26.705 | −46.787 | −14.490 | 1.00 | 36.56 | N |
| ATOM | 4649 | CA | SER | A | 578 | 25.767 | −46.385 | −13.444 | 1.00 | 34.99 | C |
| ATOM | 4650 | CB | SER | A | 578 | 25.731 | −44.870 | −13.340 | 1.00 | 33.78 | C |
| ATOM | 4651 | OG | SER | A | 578 | 26.697 | −44.407 | −12.414 | 1.00 | 38.25 | O |
| ATOM | 4652 | C | SER | A | 578 | 24.336 | −46.947 | −13.581 | 1.00 | 35.47 | C |
| ATOM | 4653 | O | SER | A | 578 | 23.668 | −47.230 | −12.574 | 1.00 | 35.32 | O |
| ATOM | 4654 | N | TRP | A | 579 | 23.892 | −47.221 | −14.809 | 1.00 | 33.06 | N |
| ATOM | 4655 | CA | TRP | A | 579 | 22.605 | −47.849 | −15.042 | 1.00 | 36.72 | C |
| ATOM | 4656 | CB | TRP | A | 579 | 21.627 | −46.908 | −15.750 | 1.00 | 35.28 | C |
| ATOM | 4657 | CG | TRP | A | 579 | 21.341 | −45.657 | −15.009 | 1.00 | 37.41 | C |
| ATOM | 4658 | CD1 | TRP | A | 579 | 20.411 | −45.488 | −14.037 | 1.00 | 40.14 | C |
| ATOM | 4659 | NE1 | TRP | A | 579 | 20.407 | −44.383 | −13.590 | 1.00 | 40.42 | N |
| ATOM | 4660 | CE2 | TRP | A | 579 | 21.361 | −43.484 | −14.277 | 1.00 | 39.25 | C |
| ATOM | 4661 | CD2 | TRP | A | 579 | 21.971 | −44.380 | −15.182 | 1.00 | 38.00 | C |
| ATOM | 4662 | CE3 | TRP | A | 579 | 22.992 | −43.907 | −15.999 | 1.00 | 40.06 | C |
| ATOM | 4663 | CZ3 | TRP | A | 579 | 23.359 | −42.565 | −15.898 | 1.00 | 39.21 | C |
| ATOM | 4664 | CH2 | TRP | A | 579 | 22.729 | −41.706 | −14.999 | 1.00 | 38.40 | C |
| ATOM | 4665 | CZ2 | TRP | A | 579 | 21.736 | −42.144 | −14.178 | 1.00 | 39.15 | C |
| ATOM | 4666 | C | TRP | A | 579 | 22.730 | −49.110 | −15.863 | 1.00 | 35.78 | C |
| ATOM | 4667 | O | TRP | A | 579 | 23.273 | −49.097 | −16.954 | 1.00 | 37.63 | O |
| ATOM | 4668 | N | PHE | A | 580 | 22.196 | −50.202 | −15.329 | 1.00 | 37.09 | N |
| ATOM | 4669 | CA | PHE | A | 580 | 21.918 | −51.374 | −16.131 | 1.00 | 32.82 | C |
| ATOM | 4670 | CB | PHE | A | 580 | 21.878 | −52.613 | −15.245 | 1.00 | 33.81 | C |
| ATOM | 4671 | CG | PHE | A | 580 | 23.210 | −53.602 | −14.671 | 1.00 | 32.62 | C |
| ATOM | 4672 | CD1 | PHE | A | 580 | 24.389 | −52.710 | −15.346 | 1.00 | 33.84 | C |
| ATOM | 4673 | CE1 | PHE | A | 580 | 25.619 | −53.093 | −14.820 | 1.00 | 35.72 | C |
| ATOM | 4674 | CZ | PHE | A | 580 | 25.681 | −53.779 | −13.616 | 1.00 | 34.17 | C |
| ATOM | 4675 | CE2 | PHE | A | 580 | 24.501 | −54.101 | −12.950 | 1.00 | 36.40 | C |
| ATOM | 4676 | CD2 | PHE | A | 580 | 23.278 | −53.723 | −13.485 | 1.00 | 32.25 | C |
| ATOM | 4677 | C | PHE | A | 580 | 20.565 | −51.168 | −16.821 | 1.00 | 31.25 | C |
| ATOM | 4678 | O | PHE | A | 580 | 19.775 | −50.316 | −16.394 | 1.00 | 28.16 | O |
| ATOM | 4679 | N | PRO | A | 581 | 20.297 | −51.947 | −17.890 | 1.00 | 31.63 | N |
| ATOM | 4680 | CA | PRO | A | 581 | 18.976 | −52.013 | −18.493 | 1.00 | 29.14 | C |
| ATOM | 4681 | CB | PRO | A | 581 | 19.138 | −53.093 | −19.560 | 1.00 | 30.28 | C |
| ATOM | 4682 | CG | PRO | A | 581 | 20.581 | −53.083 | −19.930 | 1.00 | 32.23 | C |
| ATOM | 4683 | CD | PRO | A | 581 | 21.282 | −52.768 | −18.632 | 1.00 | 31.21 | C |
| ATOM | 4684 | C | PRO | A | 581 | 17.879 | −52.437 | −17.497 | 1.00 | 31.91 | C |
| ATOM | 4685 | O | PRO | A | 581 | 18.171 | −52.992 | −16.417 | 1.00 | 30.17 | O |
| ATOM | 4686 | N | ILE | A | 582 | 16.625 | −52.166 | −17.863 | 1.00 | 30.04 | N |
| ATOM | 4687 | CA | ILE | A | 582 | 15.490 | −52.601 | −17.073 | 1.00 | 30.27 | C |
| ATOM | 4688 | CB | ILE | A | 582 | 14.559 | −51.408 | −16.716 | 1.00 | 29.87 | C |
| ATOM | 4689 | CG1 | ILE | A | 582 | 13.934 | −51.621 | −15.335 | 1.00 | 31.76 | C |
| ATOM | 4690 | CD1 | ILE | A | 582 | 13.257 | −50.423 | −14.714 | 1.00 | 30.06 | C |
| ATOM | 4691 | CG2 | ILE | A | 582 | 13.458 | −51.280 | −17.739 | 1.00 | 30.30 | C |
| ATOM | 4692 | C | ILE | A | 582 | 14.701 | −53.711 | −17.807 | 1.00 | 30.18 | C |
| ATOM | 4693 | O | ILE | A | 582 | 14.640 | −53.746 | −19.032 | 1.00 | 28.98 | O |
| ATOM | 4694 | N | LEU | A | 583 | 14.078 | −54.613 | −17.059 | 1.00 | 30.84 | N |
| ATOM | 4695 | CA | LEU | A | 583 | 13.297 | −55.654 | −17.703 | 1.00 | 31.06 | C |
| ATOM | 4696 | CB | LEU | A | 583 | 13.865 | −57.046 | −17.408 | 1.00 | 32.07 | C |
| ATOM | 4697 | CG | LEU | A | 583 | 12.971 | −58.248 | −17.763 | 1.00 | 35.14 | C |
| ATOM | 4698 | CD1 | LEU | A | 583 | 12.582 | −58.265 | −19.236 | 1.00 | 32.31 | C |
| ATOM | 4699 | CD2 | LEU | A | 583 | 13.741 | −59.503 | −17.405 | 1.00 | 34.76 | C |
| ATOM | 4700 | C | LEU | A | 583 | 11.826 | −55.584 | −17.321 | 1.00 | 28.77 | C |
| ATOM | 4701 | O | LEU | A | 583 | 11.439 | −55.627 | −16.153 | 1.00 | 25.49 | O |
| ATOM | 4702 | N | PHE | A | 584 | 11.005 | −55.486 | −18.335 | 1.00 | 28.79 | N |
| ATOM | 4703 | CA | PHE | A | 584 | 9.576 | −55.681 | −18.150 | 1.00 | 27.90 | C |
| ATOM | 4704 | CB | PHE | A | 584 | 8.826 | −54.615 | −18.947 | 1.00 | 29.29 | C |
| ATOM | 4705 | CG | PHE | A | 584 | 8.821 | −53.273 | −18.292 | 1.00 | 31.25 | C |
| ATOM | 4706 | CD1 | PHE | A | 584 | 7.904 | −52.986 | −17.263 | 1.00 | 35.48 | C |
| ATOM | 4707 | CE1 | PHE | A | 584 | 7.880 | −51.722 | −16.658 | 1.00 | 36.00 | C |
| ATOM | 4708 | CZ | PHE | A | 584 | 8.777 | −50.746 | −17.082 | 1.00 | 33.80 | C |
| ATOM | 4709 | CE2 | PHE | A | 584 | 9.681 | −51.019 | −18.112 | 1.00 | 31.95 | C |
| ATOM | 4710 | CD2 | PHE | A | 584 | 9.685 | −52.273 | −18.719 | 1.00 | 31.91 | C |
| ATOM | 4711 | C | PHE | A | 584 | 9.246 | −57.090 | −18.652 | 1.00 | 26.33 | C |
| ATOM | 4712 | O | PHE | A | 584 | 9.364 | −57.352 | −19.840 | 1.00 | 22.93 | O |
| ATOM | 4713 | N | PRO | A | 585 | 8.910 | −58.014 | −17.742 | 1.00 | 27.86 | N |
| ATOM | 4714 | CA | PRO | A | 585 | 8.477 | −59.363 | −18.076 | 1.00 | 30.72 | C |
| ATOM | 4715 | CB | PRO | A | 585 | 8.483 | −60.097 | −16.730 | 1.00 | 30.16 | C |
| ATOM | 4716 | CG | PRO | A | 585 | 8.994 | −39.128 | −15.708 | 1.00 | 31.56 | C |
| ATOM | 4717 | CD | PRO | A | 585 | 8.839 | −57.763 | −16.294 | 1.00 | 32.21 | C |
| ATOM | 4718 | C | PRO | A | 585 | 7.052 | −59.362 | −18.604 | 1.00 | 33.64 | C |
| ATOM | 4719 | O | PRO | A | 585 | 6.357 | −58.353 | −18.454 | 1.00 | 37.92 | O |
| ATOM | 4720 | N | ILE | A | 586 | 6.644 | −60.472 | −19.233 | 1.00 | 33.99 | N |
| ATOM | 4721 | CA | ILE | A | 586 | 5.219 | −60.801 | −19.444 | 1.00 | 35.82 | C |
| ATOM | 4722 | CB | ILE | A | 586 | 4.795 | −60.654 | −20.939 | 1.00 | 32.51 | C |
| ATOM | 4723 | CG1 | ILE | A | 586 | 5.635 | −61.549 | −21.842 | 1.00 | 32.89 | C |
| ATOM | 4724 | CD1 | ILE | A | 586 | 4.960 | −61.882 | −23.162 | 1.00 | 30.42 | C |
| ATOM | 4725 | CG2 | ILE | A | 586 | 4.903 | −59.204 | −21.411 | 1.00 | 28.88 | C |
| ATOM | 4726 | C | ILE | A | 586 | 4.867 | −62.203 | −18.861 | 1.00 | 37.85 | C |
| ATOM | 4727 | O | ILE | A | 586 | 5.680 | −63.123 | −18.925 | 1.00 | 42.70 | O |
| ATOM | 4728 | N | LYS | A | 587 | 3.668 | −62.356 | −18.294 | 1.00 | 39.46 | N |
| ATOM | 4729 | CA | LYS | A | 587 | 3.228 | −63.604 | −17.638 | 1.00 | 38.62 | C |
| ATOM | 4730 | CB | LYS | A | 587 | 1.758 | −63.535 | −17.233 | 1.00 | 38.40 | C |
| ATOM | 4731 | CG | LYS | A | 587 | 1.530 | −63.021 | −15.834 | 1.00 | 42.66 | C |
| ATOM | 4732 | CD | LYS | A | 587 | 0.124 | −62.459 | −15.678 | 1.00 | 44.02 | C |
| ATOM | 4733 | CE | LYS | A | 587 | −0.090 | −61.901 | −14.273 | 1.00 | 47.08 | C |
| ATOM | 4734 | NZ | LYS | A | 587 | −1.455 | −62.210 | −13.729 | 1.00 | 52.67 | N |
| ATOM | 4735 | C | LYS | A | 587 | 3.403 | −64.858 | −18.475 | 1.00 | 41.64 | C |
| ATOM | 4736 | O | LYS | A | 587 | 3.846 | −65.888 | −17.950 | 1.00 | 43.44 | O |
| ATOM | 4737 | N | GLN | A | 588 | 3.042 | −64.773 | −19.757 | 1.00 | 37.57 | N |
| ATOM | 4738 | CA | GLN | A | 588 | 3.110 | −65.905 | −20.647 | 1.00 | 35.17 | C |
| ATOM | 4739 | CB | GLN | A | 588 | 1.703 | −66.343 | −21.086 | 1.00 | 35.67 | C |
| ATOM | 4740 | CG | GLN | A | 588 | 0.975 | −67.139 | −20.010 | 1.00 | 37.57 | C |
| ATOM | 4741 | CD | GLN | A | 588 | 1.569 | −68.538 | −19.817 | 1.00 | 40.46 | C |
| ATOM | 4742 | OE1 | GLN | A | 588 | 1.641 | −69.349 | −20.762 | 1.00 | 37.67 | O |
| ATOM | 4743 | NE2 | GLN | A | 588 | 1.983 | −68.833 | −18.590 | 1.00 | 40.56 | N |
| ATOM | 4744 | C | GLN | A | 588 | 3.959 | −65.592 | −21.852 | 1.00 | 36.10 | C |
| ATOM | 4745 | O | GLN | A | 588 | 3.768 | −64.579 | −22.494 | 1.00 | 37.94 | O |
| ATOM | 4746 | N | PRO | A | 589 | 4.930 | −66.459 | −22.154 | 1.00 | 37.77 | N |
| ATOM | 4747 | CA | PRO | A | 589 | 5.727 | −66.401 | −23.375 | 1.00 | 34.97 | C |
| ATOM | 4748 | CB | PRO | A | 589 | 6.367 | −67.778 | −23.409 | 1.00 | 36.69 | C |
| ATOM | 4749 | CG | PRO | A | 589 | 6.431 | −68.211 | −21.984 | 1.00 | 35.55 | C |
| ATOM | 4750 | CD | PRO | A | 589 | 5.214 | −67.661 | −21.352 | 1.00 | 38.03 | C |
| ATOM | 4751 | C | PRO | A | 589 | 4.772 | −66.310 | −24.560 | 1.00 | 36.58 | C |
| ATOM | 4752 | O | PRO | A | 589 | 3.713 | −66.942 | −24.537 | 1.00 | 34.02 | O |
| ATOM | 4753 | N | ILE | A | 590 | 5.127 | −65.557 | −25.587 | 1.00 | 36.10 | N |
| ATOM | 4754 | CA | ILE | A | 590 | 4.288 | −65.493 | −26.768 | 1.00 | 36.21 | C |
| ATOM | 4755 | CB | ILE | A | 590 | 3.700 | −64.089 | −27.008 | 1.00 | 35.72 | C |
| ATOM | 4756 | CG1 | ILE | A | 590 | 2.765 | −63.648 | −25.881 | 1.00 | 35.44 | C |
| ATOM | 4757 | CD1 | ILE | A | 590 | 2.426 | −62.163 | −25.949 | 1.00 | 34.24 | C |
| ATOM | 4758 | CG2 | ILE | A | 590 | 2.975 | −64.046 | −28.350 | 1.00 | 36.86 | C |
| ATOM | 4759 | C | ILE | A | 590 | 5.140 | −65.809 | −27.976 | 1.00 | 38.80 | C |
| ATOM | 4760 | O | ILE | A | 590 | 6.205 | −65.210 | −28.159 | 1.00 | 40.26 | O |
| ATOM | 4761 | N | THR | A | 591 | 4.647 | −66.722 | −28.813 | 1.00 | 40.29 | N |
| ATOM | 4762 | CA | THR | A | 591 | 5.340 | −67.126 | −30.033 | 1.00 | 40.21 | C |
| ATOM | 4763 | CB | THR | A | 591 | 4.791 | −68.465 | −30.551 | 1.00 | 41.07 | C |
| ATOM | 4764 | OG1 | THR | A | 591 | 5.168 | −69.512 | −29.645 | 1.00 | 39.01 | O |
| ATOM | 4765 | CG2 | THR | A | 591 | 5.310 | −68.768 | −31.984 | 1.00 | 38.32 | C |
| ATOM | 4766 | C | THR | A | 591 | 5.157 | −66.080 | −31.108 | 1.00 | 42.39 | C |
| ATOM | 4767 | O | THR | A | 591 | 4.026 | −65.699 | −31.428 | 1.00 | 43.38 | O |
| ATOM | 4768 | N | VAL | A | 592 | 6.269 | −65.613 | −31.661 | 1.00 | 40.06 | N |
| ATOM | 4769 | CA | VAL | A | 592 | 6.223 | −64.683 | −32.773 | 1.00 | 37.48 | C |
| ATOM | 4770 | CB | VAL | A | 592 | 6.943 | −63.366 | −32.424 | 1.00 | 34.09 | C |
| ATOM | 4771 | CG1 | VAL | A | 592 | 6.916 | −62.349 | −33.577 | 1.00 | 32.38 | C |
| ATOM | 4772 | CG2 | VAL | A | 592 | 6.368 | −62.753 | −31.166 | 1.00 | 31.00 | C |
| ATOM | 4773 | C | VAL | A | 592 | 6.879 | −65.365 | −33.970 | 1.00 | 46.22 | C |
| ATOM | 4774 | O | VAL | A | 592 | 7.879 | −66.074 | −33.818 | 1.00 | 55.51 | O |
| ATOM | 4775 | N | ARG | A | 593 | 6.306 | −65.160 | −35.152 | 1.00 | 50.97 | N |
| ATOM | 4776 | CA | ARG | A | 593 | 6.851 | −65.680 | −36.401 | 1.00 | 52.82 | C |
| ATOM | 4777 | CB | ARG | A | 593 | 5.754 | −66.298 | −37.257 | 1.00 | 52.58 | C |
| ATOM | 4778 | CG | ARG | A | 593 | 5.257 | −67.653 | −36.789 | 1.00 | 61.00 | C |
| ATOM | 4779 | CD | ARG | A | 593 | 4.171 | −68.226 | −37.742 | 1.00 | 73.00 | C |
| ATOM | 4780 | NE | ARG | A | 593 | 3.336 | −66.990 | −38.161 | 1.00 | 83.14 | N |
| ATOM | 4781 | CZ | ARG | A | 593 | 2.444 | −67.006 | −39.151 | 1.00 | 86.90 | C |
| ATOM | 4782 | NH1 | ARG | A | 593 | 2.227 | −68.115 | −39.866 | 1.00 | 88.00 | N |
| ATOM | 4783 | NH2 | ARG | A | 593 | 1.765 | −65.894 | −39.427 | 1.00 | 85.25 | N |
| ATOM | 4784 | C | ARG | A | 593 | 7.533 | −64.601 | −37.225 | 1.00 | 54.26 | C |
| ATOM | 4785 | O | ARG | A | 593 | 7.146 | −63.423 | −37.206 | 1.00 | 54.61 | O |
| ATOM | 4786 | N | GLU | A | 594 | 8.531 | −65.029 | −37.984 | 1.00 | 49.91 | N |
| ATOM | 4787 | CA | GLU | A | 594 | 9.227 | −64.154 | −38.884 | 1.00 | 51.62 | C |
| ATOM | 4788 | CB | GLU | A | 594 | 10.177 | −64.969 | −39.754 | 1.00 | 60.23 | C |
| ATOM | 4789 | CG | GLU | A | 594 | 10.954 | −64.131 | −40.756 | 1.00 | 73.65 | C |
| ATOM | 4790 | CD | GLU | A | 594 | 10.850 | −64.669 | −42.171 | 1.00 | 83.56 | C |
| ATOM | 4791 | OE1 | GLU | A | 594 | 10.185 | −65.719 | −42.371 | 1.00 | 87.61 | O |
| ATOM | 4792 | OE2 | GLU | A | 594 | 11.429 | −64.032 | −43.080 | 1.00 | 81.54 | O |
| ATOM | 4793 | C | GLU | A | 594 | 8.229 | −63.408 | −39.743 | 1.00 | 49.64 | C |
| ATOM | 4794 | O | GLU | A | 594 | 7.284 | −64.000 | −40.255 | 1.00 | 51.92 | O |
| ATOM | 4795 | N | GLY | A | 595 | 8.441 | −62.107 | −39.892 | 1.00 | 48.83 | N |
| ATOM | 4796 | CA | GLY | A | 595 | 7.547 | −61.252 | −40.680 | 1.00 | 49.47 | C |
| ATOM | 4797 | C | GLY | A | 595 | 6.475 | −60.543 | −39.859 | 1.00 | 48.86 | C |
| ATOM | 4798 | O | GLY | A | 595 | 5.949 | −59.518 | −40.286 | 1.00 | 47.80 | O |
| ATOM | 4799 | N | GLN | A | 596 | 6.161 | −61.081 | −38.678 | 1.00 | 45.91 | N |
| ATOM | 4800 | CA | GLN | A | 596 | 5.164 | −60.484 | −37.794 | 1.00 | 44.36 | C |
| ATOM | 4801 | CB | GLN | A | 596 | 4.672 | −61.518 | −36.797 | 1.00 | 44.70 | C |
| ATOM | 4802 | CG | GLN | A | 596 | 3.810 | −62.589 | −37.446 | 1.00 | 42.00 | C |
| ATOM | 4803 | CD | GLN | A | 596 | 3.225 | −63.553 | −36.447 | 1.00 | 43.69 | C |
| ATOM | 4804 | OE1 | GLN | A | 596 | 3.842 | −63.873 | −35.422 | 1.00 | 44.56 | O |
| ATOM | 4805 | NE2 | GLN | A | 596 | 2.014 | −64.016 | −36.726 | 1.00 | 43.80 | N |
| ATOM | 4806 | C | GLN | A | 596 | 5.596 | −59.202 | −37.066 | 1.00 | 44.96 | C |
| ATOM | 4807 | O | GLN | A | 596 | 6.791 | −58.907 | −36.916 | 1.00 | 44.21 | O |
| ATOM | 4808 | N | THR | A | 597 | 4.601 | −58.420 | −36.656 | 1.00 | 42.62 | N |
| ATOM | 4809 | CA | THR | A | 597 | 4.834 | −57.256 | −35.817 | 1.00 | 44.65 | C |
| ATOM | 4810 | CB | THR | A | 597 | 4.023 | −56.048 | −36.305 | 1.00 | 43.57 | C |
| ATOM | 4811 | OG1 | THR | A | 597 | 4.453 | −55.712 | −37.615 | 1.00 | 42.79 | O |
| ATOM | 4812 | CG2 | THR | A | 597 | 4.262 | −54.842 | −35.441 | 1.00 | 45.47 | C |
| ATOM | 4813 | C | THR | A | 597 | 4.470 | −57.592 | −34.368 | 1.00 | 44.82 | C |
| ATOM | 4814 | O | THR | A | 597 | 3.432 | −58.210 | −34.103 | 1.00 | 48.73 | O |
| ATOM | 4815 | N | ILE | A | 598 | 5.345 | −57.233 | −33.438 | 1.00 | 42.27 | N |
| ATOM | 4816 | CA | ILE | A | 598 | 4.970 | −57.261 | −32.033 | 1.00 | 40.02 | C |
| ATOM | 4817 | CB | ILE | A | 598 | 6.138 | −57.505 | −31.100 | 1.00 | 38.63 | C |
| ATOM | 4818 | CG1 | ILE | A | 598 | 6.586 | −58.963 | −31.187 | 1.00 | 40.93 | C |
| ATOM | 4819 | CD1 | ILE | A | 598 | 8.044 | −59.167 | −30.812 | 1.00 | 39.40 | C |
| ATOM | 4820 | CG2 | ILE | A | 598 | 5.731 | −57.154 | −29.682 | 1.00 | 38.83 | C |
| ATOM | 4821 | C | ILE | A | 598 | 4.444 | −55.889 | −31.724 | 1.00 | 38.22 | C |
| ATOM | 4822 | O | ILE | A | 598 | 5.150 | −54.905 | −31.958 | 1.00 | 35.75 | O |
| ATOM | 4823 | N | CYS | A | 599 | 3.197 | −55.829 | −31.236 | 1.00 | 35.61 | N |
| ATOM | 4824 | CA | CYS | A | 599 | 2.625 | −54.585 | −30.753 | 1.00 | 31.06 | C |
| ATOM | 4825 | CB | CYS | A | 599 | 1.241 | −54.334 | −31.335 | 1.00 | 35.89 | C |
| ATOM | 4826 | SG | CYS | A | 599 | 0.636 | −52.683 | −30.916 | 1.00 | 41.08 | S |
| ATOM | 4827 | C | CYS | A | 599 | 2.538 | −54.637 | −29.249 | 1.00 | 30.23 | C |
| ATOM | 4828 | O | CYS | A | 599 | 2.042 | −55.611 | −28.679 | 1.00 | 29.81 | O |
| ATOM | 4829 | N | VAL | A | 600 | 3.057 | −53.601 | −28.598 | 1.00 | 28.90 | N |
| ATOM | 4830 | CA | VAL | A | 600 | 2.987 | −53.498 | −27.156 | 1.00 | 27.20 | C |
| ATOM | 4831 | CB | VAL | A | 600 | 4.373 | −53.606 | −26.510 | 1.00 | 27.37 | C |
| ATOM | 4832 | CG1 | VAL | A | 600 | 5.113 | −52.285 | −26.666 | 1.00 | 26.91 | C |
| ATOM | 4833 | CG2 | VAL | A | 600 | 4.227 | −53.913 | −25.027 | 1.00 | 29.09 | C |
| ATOM | 4834 | C | VAL | A | 600 | 2.403 | −52.131 | −26.793 | 1.00 | 27.09 | C |
| ATOM | 4835 | O | VAL | A | 600 | 2.377 | −51.208 | −27.603 | 1.00 | 26.91 | O |
| ATOM | 4836 | N | ARG | A | 601 | 1.954 | −51.995 | −25.558 | 1.00 | 28.21 | N |
| ATOM | 4837 | CA | ARG | A | 601 | 1.301 | −50.774 | −25.134 | 1.00 | 29.32 | C |
| ATOM | 4838 | CB | ARG | A | 601 | −0.217 | −50.962 | −25.180 | 1.00 | 30.55 | C |
| ATOM | 4839 | CG | ARG | A | 601 | −0.631 | −51.459 | −26.566 | 1.00 | 34.08 | C |
| ATOM | 4840 | CD | ARG | A | 601 | −1.802 | −50.742 | −27.198 | 1.00 | 37.18 | C |
| ATOM | 4841 | NE | ARG | A | 601 | −2.250 | −51.520 | −28.350 | 1.00 | 40.32 | N |
| ATOM | 4842 | CE | ARG | A | 601 | −3.126 | −51.098 | −29.250 | 1.00 | 40.09 | C |
| ATOM | 4843 | NH1 | ARG | A | 601 | −3.687 | −49.908 | −29.142 | 1.00 | 38.52 | N |
| ATOM | 4844 | NH2 | ARG | A | 601 | −3.473 | −51.886 | −30.245 | 1.00 | 44.37 | N |
| ATOM | 4845 | C | ARG | A | 601 | 1.749 | −50.553 | −23.751 | 1.00 | 28.44 | C |
| ATOM | 4846 | O | ARG | A | 601 | 1.690 | −51.472 | −22.939 | 1.00 | 31.36 | O |
| ATOM | 4847 | N | PHE | A | 602 | 2.232 | −49.351 | −23.477 | 1.00 | 27.22 | N |
| ATOM | 4848 | CA | PHE | A | 602 | 2.656 | −49.005 | −22.140 | 1.00 | 27.82 | C |
| ATOM | 4849 | CB | PHE | A | 602 | 4.178 | −48.687 | −22.104 | 1.00 | 29.30 | C |
| ATOM | 4850 | CG | PHE | A | 602 | 5.019 | −49.869 | −21.716 | 1.00 | 29.85 | C |
| ATOM | 4851 | CD1 | PHE | A | 602 | 5.171 | −50.939 | −22.584 | 1.00 | 30.20 | C |
| ATOM | 4852 | CE1 | PHE | A | 602 | 5.934 | −52.038 | −22.209 | 1.00 | 31.47 | C |
| ATOM | 4853 | CZ | PHE | A | 602 | 6.530 | −52.076 | −20.958 | 1.00 | 29.86 | C |
| ATOM | 4854 | CE2 | PHE | A | 602 | 6.386 | −51.016 | −20.086 | 1.00 | 28.02 | C |
| ATOM | 4855 | CD2 | PHE | A | 602 | 5.631 | −49.973 | −20.470 | 1.00 | 30.02 | C |
| ATOM | 4856 | C | PHE | A | 602 | 1.890 | −47.823 | −21.638 | 1.00 | 28.17 | C |
| ATOM | 4857 | O | PHE | A | 602 | 1.708 | −46.852 | −22.372 | 1.00 | 31.12 | O |
| ATOM | 4858 | N | TRP | A | 603 | 1.493 | −47.880 | −20.372 | 1.00 | 28.08 | N |
| ATOM | 4859 | CA | TRP | A | 603 | 0.920 | −46.723 | −19.712 | 1.00 | 29.76 | C |
| ATOM | 4860 | CB | TRP | A | 603 | −0.498 | −46.999 | −19.209 | 1.00 | 29.27 | C |
| ATOM | 4861 | CG | TRP | A | 603 | −1.522 | −47.381 | −20.199 | 1.00 | 29.41 | C |
| ATOM | 4862 | CD1 | TRP | A | 603 | −2.525 | −46.592 | −20.666 | 1.00 | 34.06 | C |
| ATOM | 4863 | NE1 | TRP | A | 603 | −3.358 | −47.302 | −21.505 | 1.00 | 32.68 | N |
| ATOM | 4864 | CE2 | TRP | A | 603 | −2.901 | −48.589 | −21.592 | 1.00 | 32.38 | C |
| ATOM | 4865 | CD2 | TRP | A | 603 | −1.745 | −48.684 | −20.767 | 1.00 | 33.00 | C |
| ATOM | 4866 | CE3 | TRP | A | 603 | −1.076 | −49.919 | −20.679 | 1.00 | 30.10 | C |
| ATOM | 4867 | CZ3 | TRP | A | 603 | −1.570 | −50.997 | −21.402 | 1.00 | 30.19 | C |
| ATOM | 4868 | CE2 | TRP | A | 603 | −2.726 | −50.865 | −22.212 | 1.00 | 31.82 | C |
| ATOM | 4869 | CZ2 | TRP | A | 603 | −3.383 | −49.669 | −22.335 | 1.00 | 30.03 | C |
| ATOM | 4870 | C | TRP | A | 603 | 1.740 | −46.301 | −18.520 | 1.00 | 30.92 | C |
| ATOM | 4871 | O | TRP | A | 603 | 2.204 | −47.137 | −17.738 | 1.00 | 34.13 | O |
| ATOM | 4872 | N | ARG | A | 604 | 1.887 | −44.994 | −18.334 | 1.00 | 30.95 | N |
| ATOM | 4873 | CA | ARG | A | 604 | 2.431 | −44.516 | −17.059 | 1.00 | 31.62 | C |
| ATOM | 4874 | CB | ARG | A | 604 | 3.499 | −43.442 | −17.277 | 1.00 | 29.97 | C |
| ATOM | 4875 | CG | ARG | A | 604 | 4.171 | −42.973 | −16.009 | 1.00 | 30.02 | C |
| ATOM | 4876 | CD | ARG | A | 604 | 5.158 | −41.369 | −16.367 | 1.00 | 32.29 | C |
| ATOM | 4877 | NE | ARG | A | 604 | 6.188 | −41.571 | −15.368 | 1.00 | 32.23 | N |
| ATOM | 4878 | CZ | ARG | A | 604 | 6.108 | −40.340 | −14.325 | 1.00 | 32.29 | C |
| ATOM | 4879 | NH1 | ARG | A | 604 | 5.031 | −40.117 | −14.123 | 1.00 | 29.49 | N |
| ATOM | 4880 | NH2 | ARG | A | 604 | 7.125 | −40.735 | −13.473 | 1.00 | 30.96 | N |
| ATOM | 4881 | C | ARG | A | 604 | 1.251 | −43.929 | −16.316 | 1.00 | 28.77 | C |
| ATOM | 4882 | O | ARG | A | 604 | 0.625 | −43.057 | −16.840 | 1.00 | 27.90 | O |
| ATOM | 4883 | N | CYS | A | 605 | 1.041 | −44.365 | −15.079 | 1.00 | 29.39 | N |
| ATOM | 4884 | CA | CYS | A | 605 | −0.139 | −44.095 | −14.280 | 1.00 | 32.34 | C |
| ATOM | 4885 | CB | CYS | A | 605 | −0.890 | −45.407 | −14.020 | 1.00 | 31.30 | C |
| ATOM | 4886 | SG | CYS | A | 605 | −1.411 | −46.261 | −15.524 | 1.00 | 39.03 | S |
| ATOM | 4887 | C | CYS | A | 605 | 0.201 | −43.464 | −12.936 | 1.00 | 34.02 | C |
| ATOM | 4888 | O | CYS | A | 605 | 1.351 | −43.683 | −12.447 | 1.00 | 30.58 | O |
| ATOM | 4889 | N | SER | A | 606 | −0.800 | −42.809 | −12.327 | 1.00 | 32.64 | N |
| ATOM | 4890 | CA | SER | A | 606 | −0.583 | −42.176 | −11.032 | 1.00 | 35.70 | C |
| ATOM | 4891 | CB | SER | A | 606 | 0.276 | −40.918 | −11.165 | 1.00 | 33.50 | C |
| ATOM | 4892 | OG | SER | A | 606 | −0.252 | −40.063 | −12.130 | 1.00 | 33.87 | O |
| ATOM | 4893 | C | SER | A | 606 | −1.774 | −41.840 | −10.159 | 1.00 | 39.90 | C |
| ATOM | 4894 | O | SER | A | 606 | −2.903 | −41.708 | −10.623 | 1.00 | 40.91 | O |
| ATOM | 4895 | N | ASN | A | 607 | −1.439 | −41.728 | −8.874 | 1.00 | 41.51 | N |
| ATOM | 4896 | CA | ASN | A | 607 | −2.245 | −41.283 | −7.762 | 1.00 | 41.72 | C |
| ATOM | 4897 | CB | ASN | A | 607 | −2.115 | −42.293 | −6.606 | 1.00 | 41.12 | C |
| ATOM | 4898 | CG | ASN | A | 607 | −3.228 | −43.282 | −6.590 | 1.00 | 45.65 | C |
| ATOM | 4899 | OD1 | ASN | A | 607 | −4.248 | −43.052 | −7.219 | 1.00 | 54.04 | O |
| ATOM | 4900 | ND2 | ASN | A | 607 | −3.052 | −44.891 | −5.886 | 1.00 | 45.49 | N |
| ATOM | 4901 | C | ASN | A | 607 | −1.549 | −40.053 | −7.267 | 1.00 | 43.27 | C |
| ATOM | 4902 | O | ASN | A | 607 | −0.445 | −39.723 | −7.732 | 1.00 | 46.72 | O |
| ATOM | 4903 | N | SER | A | 608 | −2.138 | −39.423 | −6.263 | 1.00 | 41.81 | N |
| ATOM | 4904 | CA | SER | A | 608 | −1.395 | −38.472 | −5.456 | 1.00 | 48.56 | C |
| ATOM | 4905 | CB | SER | A | 608 | −2.344 | −37.657 | −4.591 | 1.00 | 47.17 | C |
| ATOM | 4906 | OG | SER | A | 608 | −2.970 | −38.916 | −3.661 | 1.00 | 47.12 | O |
| ATOM | 4907 | C | SER | A | 608 | −0.397 | −39.211 | −4.544 | 1.00 | 46.92 | C |
| ATOM | 4908 | O | SER | A | 608 | 0.412 | −38.183 | −3.892 | 1.00 | 46.75 | O |
| ATOM | 4909 | N | LYS | A | 609 | −0.469 | −40.537 | −4.498 | 1.00 | 47.67 | N |
| ATOM | 4910 | CA | LYS | A | 609 | 0.375 | −41.318 | −3.597 | 1.00 | 45.38 | C |
| ATOM | 4911 | CB | LYS | A | 609 | −0.474 | −42.236 | −2.717 | 1.00 | 48.04 | C |
| ATOM | 4912 | CG | LYS | A | 609 | −1.327 | −41.462 | −1.719 | 1.00 | 51.99 | C |
| ATOM | 4913 | CD | LYS | A | 609 | −2.304 | −42.149 | −0.968 | 1.00 | 58.45 | C |
| ATOM | 4914 | CE | LYS | A | 609 | −3.005 | −41.542 | 0.113 | 1.00 | 62.26 | C |
| ATOM | 4915 | NZ | LYS | A | 609 | −3.714 | −42.405 | 1.098 | 1.00 | 68.97 | N |
| ATOM | 4916 | C | LYS | A | 609 | 1.455 | −42.210 | −4.312 | 1.00 | 43.47 | C |
| ATOM | 4917 | O | LYS | A | 609 | 2.539 | −42.317 | −3.756 | 1.00 | 44.38 | O |
| ATOM | 4918 | N | LYS | A | 610 | 1.173 | −42.546 | −5.541 | 1.00 | 39.76 | N |
| ATOM | 4919 | CA | LYS | A | 610 | 2.090 | −43.450 | −6.247 | 1.00 | 34.16 | C |
| ATOM | 4920 | CB | LYS | A | 610 | 1.863 | −44.884 | −5.808 | 1.00 | 35.25 | C |
| ATOM | 4921 | CG | LYS | A | 610 | 0.415 | −45.327 | −5.885 | 1.00 | 40.18 | C |
| ATOM | 4922 | CD | LYS | A | 610 | 0.090 | −46.488 | −4.949 | 1.00 | 46.15 | C |
| ATOM | 4923 | CE | LYS | A | 610 | −1.253 | −47.092 | −5.388 | 1.00 | 52.18 | C |
| ATOM | 4924 | NZ | LYS | A | 610 | −1.886 | −48.014 | −4.394 | 1.00 | 51.75 | N |
| ATOM | 4925 | C | LYS | A | 610 | 2.022 | −43.338 | −7.756 | 1.00 | 32.41 | C |
| ATOM | 4926 | O | LYS | A | 610 | 1.085 | −42.765 | −8.304 | 1.00 | 30.34 | O |
| ATOM | 4927 | N | VAL | A | 611 | 3.049 | −43.878 | −8.410 | 1.00 | 30.71 | N |
| ATOM | 4928 | CA | VAL | A | 611 | 3.200 | −43.864 | −9.867 | 1.00 | 29.29 | C |
| ATOM | 4929 | CB | VAL | A | 611 | 4.328 | −42.892 | −10.308 | 1.00 | 26.64 | C |
| ATOM | 4930 | CG1 | VAL | A | 611 | 4.514 | −42.879 | −11.823 | 1.00 | 25.87 | C |
| ATOM | 4931 | CG2 | VAL | A | 611 | 3.992 | −41.490 | −9.856 | 1.00 | 27.81 | C |
| ATOM | 4932 | C | VAL | A | 611 | 3.552 | −45.284 | −10.259 | 1.00 | 30.10 | C |
| ATOM | 4933 | O | VAL | A | 611 | 4.278 | −45.962 | −9.522 | 1.00 | 33.64 | O |
| ATOM | 4934 | N | TRP | A | 612 | 3.040 | −45.156 | −11.393 | 1.00 | 29.97 | N |
| ATOM | 4935 | CA | TRP | A | 612 | 3.306 | −47.136 | −11.807 | 1.00 | 29.28 | C |
| ATOM | 4936 | CB | TRP | A | 612 | 2.388 | −48.120 | −11.085 | 1.00 | 31.57 | C |
| ATOM | 4937 | CG | TRP | A | 612 | 0.907 | −48.007 | −11.434 | 1.00 | 34.40 | C |
| ATOM | 4938 | CD1 | TRP | A | 612 | 0.240 | −48.705 | −12.408 | 1.00 | 35.80 | C |
| ATOM | 4939 | NE1 | TRP | A | 612 | −1.100 | −48.363 | −12.406 | 1.00 | 36.76 | N |
| ATOM | 4940 | CE2 | TRP | A | 612 | −1.312 | −47.426 | −11.427 | 1.00 | 34.76 | C |
| ATOM | 4941 | CD2 | TRP | A | 612 | −0.066 | −47.175 | −10.800 | 1.00 | 32.54 | C |
| ATOM | 4942 | CE3 | TRP | A | 612 | −0.014 | −46.249 | −9.761 | 1.00 | 35.88 | C |
| ATOM | 4943 | CZ3 | TRP | A | 612 | −1.221 | −45.596 | −9.373 | 1.00 | 35.89 | C |
| ATOM | 4944 | CH2 | TRP | A | 612 | −2.426 | −45.872 | −10.024 | 1.00 | 33.04 | C |
| ATOM | 4945 | CZ2 | TRP | A | 612 | −2.493 | −46.772 | −11.053 | 1.00 | 32.19 | C |
| ATOM | 4946 | C | TRP | A | 612 | 3.150 | −47.273 | −13.296 | 1.00 | 28.46 | C |
| ATOM | 4947 | O | TRP | A | 612 | 2.615 | −46.371 | −13.905 | 1.00 | 29.52 | O |
| ATOM | 4948 | N | TYR | A | 613 | 3.660 | −48.369 | −13.870 | 1.00 | 26.94 | N |
| ATOM | 4949 | CA | TYR | A | 613 | 3.452 | −48.672 | −15.267 | 1.00 | 26.79 | C |
| ATOM | 4950 | CB | TYR | A | 613 | 4.763 | −48.991 | −15.981 | 1.00 | 28.09 | C |
| ATOM | 4951 | CG | TYR | A | 613 | 5.823 | −47.918 | −15.906 | 1.00 | 30.59 | C |
| ATOM | 4952 | CD1 | TYR | A | 613 | 5.760 | −46.790 | −16.701 | 1.00 | 31.35 | C |
| ATOM | 4953 | CE1 | TYR | A | 613 | 6.726 | −45.812 | −16.628 | 1.00 | 30.54 | C |
| ATOM | 4954 | CZ | TYR | A | 613 | 7.806 | −45.989 | −15.779 | 1.00 | 33.09 | C |
| ATOM | 4955 | OH | TYR | A | 613 | 8.767 | −45.021 | −15.683 | 1.00 | 31.56 | O |
| ATOM | 4956 | CE2 | TYR | A | 613 | 7.907 | −47.110 | −14.983 | 1.00 | 31.40 | C |
| ATOM | 4957 | CD2 | TYR | A | 613 | 6.919 | −48.061 | −15.055 | 1.00 | 33.28 | C |
| ATOM | 4958 | C | TYR | A | 613 | 2.450 | −49.833 | −15.410 | 1.00 | 26.01 | C |
| ATOM | 4959 | O | TYR | A | 613 | 2.283 | −50.655 | −14.517 | 1.00 | 26.96 | O |
| ATOM | 4960 | N | GLU | A | 614 | 1.753 | −49.867 | −16.524 | 1.00 | 24.95 | N |
| ATOM | 4961 | CA | GLU | A | 614 | 0.920 | −51.019 | −16.882 | 1.00 | 26.42 | C |
| ATOM | 4962 | CB | GLU | A | 614 | −0.578 | −50.696 | −16.748 | 1.00 | 23.76 | C |
| ATOM | 4963 | CG | GLU | A | 614 | −0.931 | −49.958 | −15.460 | 1.00 | 25.71 | C |
| ATOM | 4964 | CD | GLU | A | 614 | −2.451 | −49.812 | −15.223 | 1.00 | 28.65 | C |
| ATOM | 4965 | OE1 | GLU | A | 614 | −3.210 | −49.874 | −16.198 | 1.00 | 28.30 | O |
| ATOM | 4966 | OE2 | GLU | A | 614 | −2.897 | −49.610 | −14.065 | 1.00 | 28.34 | O |
| ATOM | 4967 | C | GLU | A | 614 | 1.279 | −51.237 | −18.333 | 1.00 | 24.92 | C |
| ATOM | 4968 | O | GLU | A | 614 | 1.553 | −50.266 | −19.042 | 1.00 | 25.71 | O |
| ATOM | 4969 | N | TRP | A | 615 | 1.324 | −52.489 | −18.772 | 1.00 | 22.44 | N |
| ATOM | 4970 | CA | TRP | A | 615 | 1.684 | −52.767 | −20.159 | 1.00 | 22.31 | C |
| ATOM | 4971 | C3 | TRP | A | 615 | 3.221 | −53.010 | −20.284 | 1.00 | 19.67 | C |
| ATOM | 4972 | CG | TRP | A | 615 | 3.778 | −54.113 | −19.399 | 1.00 | 16.52 | C |
| ATOM | 4973 | CD1 | TRP | A | 615 | 4.084 | −55.373 | −19.795 | 1.00 | 15.70 | C |
| ATOM | 4974 | NE1 | TRP | A | 615 | 4.553 | −56.116 | −18.714 | 1.00 | 16.37 | N |
| ATOM | 4975 | CE2 | TRP | A | 615 | 4.629 | −55.311 | −17.613 | 1.00 | 15.84 | C |
| ATOM | 4976 | CD2 | TRP | A | 615 | 4.136 | −54.028 | −18.000 | 1.00 | 16.48 | C |
| ATOM | 4977 | CE3 | TRP | A | 615 | 4.098 | −52.994 | −17.038 | 1.00 | 17.46 | C |
| ATOM | 4978 | CZ3 | TRP | A | 615 | 4.541 | −53.271 | −15.732 | 1.00 | 17.49 | C |
| ATOM | 4979 | CH2 | TRP | A | 615 | 5.056 | −54.591 | −15.388 | 1.00 | 17.84 | C |
| ATOM | 4980 | CZ2 | TRP | A | 615 | 5.082 | −55.604 | −16.314 | 1.00 | 16.05 | C |
| ATOM | 4981 | C | TRP | A | 615 | 0.893 | −53.954 | −20.701 | 1.00 | 22.95 | C |
| ATOM | 4982 | O | TRP | A | 615 | 0.466 | −54.770 | −19.923 | 1.00 | 25.17 | O |
| ATOM | 4983 | N | ALA | A | 616 | 0.679 | −54.025 | −22.018 | 1.00 | 23.43 | N |
| ATOM | 4984 | CA | ALA | A | 616 | 0.108 | −55.217 | −22.633 | 1.00 | 27.16 | C |
| ATOM | 4985 | CB | ALA | A | 616 | −1.431 | −55.162 | −22.656 | 1.00 | 25.50 | C |
| ATOM | 4986 | C | ALA | A | 616 | 0.638 | −55.375 | −24.036 | 1.00 | 29.00 | C |
| ATOM | 4987 | O | ALA | A | 616 | 0.978 | −54.387 | −24.670 | 1.00 | 34.74 | O |
| ATOM | 4988 | N | VAL | A | 617 | 0.678 | −56.621 | −24.510 | 1.00 | 28.81 | N |
| ATOM | 4989 | CA | VAL | A | 617 | 1.000 | −56.927 | −25.883 | 1.00 | 29.23 | C |
| ATOM | 4990 | CB | VAL | A | 617 | 1.801 | −58.256 | −25.986 | 1.00 | 29.40 | C |
| ATOM | 4991 | CG1 | VAL | A | 617 | 2.138 | −58.612 | −27.435 | 1.00 | 25.91 | C |
| ATOM | 4992 | CG2 | VAL | A | 617 | 3.066 | −58.188 | −25.145 | 1.00 | 28.39 | C |
| ATOM | 4993 | C | VAL | A | 617 | −0.326 | −57.138 | −26.576 | 1.00 | 31.60 | C |
| ATOM | 4994 | O | VAL | A | 617 | −1.187 | −57.859 | −26.069 | 1.00 | 32.66 | O |
| ATOM | 4995 | N | THR | A | 618 | −0.488 | −56.534 | −27.745 | 1.00 | 32.43 | N |
| ATOM | 4996 | CA | THR | A | 618 | −1.729 | −56.688 | −28.470 | 1.00 | 37.18 | C |
| ATOM | 4997 | CB | THR | A | 618 | −2.426 | −55.331 | −28.631 | 1.00 | 36.52 | C |
| ATOM | 4998 | OG1 | THR | A | 618 | −1.536 | −54.433 | −29.297 | 1.00 | 40.33 | O |
| ATOM | 4999 | CG2 | THR | A | 618 | −2.758 | −54.745 | −27.262 | 1.00 | 33.30 | C |
| ATOM | 5000 | C | THR | A | 618 | −1.531 | −57.384 | −29.816 | 1.00 | 39.11 | C |
| ATOM | 5001 | O | THR | A | 618 | −2.498 | −57.545 | −30.524 | 1.00 | 42.16 | O |
| ATOM | 5002 | N | ALA | A | 619 | −0.279 | −57.571 | −30.174 | 1.00 | 38.66 | N |
| ATOM | 5003 | CA | ALA | A | 619 | 0.045 | −58.379 | −31.425 | 1.00 | 36.77 | C |
| ATOM | 5004 | CB | ALA | A | 619 | 0.047 | −57.418 | −32.607 | 1.00 | 34.93 | C |
| ATOM | 5005 | C | ALA | A | 619 | 1.411 | −59.960 | −31.290 | 1.00 | 40.19 | C |
| ATOM | 5006 | O | ALA | A | 619 | 2.332 | −58.461 | −30.719 | 1.00 | 39.98 | O |
| ATOM | 5007 | N | PRO | A | 620 | 1.577 | −60.266 | −31.883 | 1.00 | 39.98 | N |
| ATOM | 5008 | CA | PRO | A | 620 | 0.663 | −60.358 | −32.860 | 1.00 | 44.00 | C |
| ATOM | 5009 | CB | PRO | A | 620 | 1.527 | −61.917 | −33.543 | 1.00 | 41.97 | C |
| ATOM | 5010 | CG | PRO | A | 620 | 2.453 | −62.366 | −32.474 | 1.00 | 40.49 | C |
| ATOM | 5011 | CD | PRO | A | 620 | 2.726 | −61.153 | −31.621 | 1.00 | 42.13 | C |
| ATOM | 5012 | C | PRO | A | 620 | −0.514 | −61.514 | −32.191 | 1.00 | 43.86 | C |
| ATOM | 5013 | O | PRO | A | 620 | −1.420 | −61.960 | −32.869 | 1.00 | 53.37 | O |
| ATOM | 5014 | N | VAL | A | 621 | −0.477 | −61.551 | −30.867 | 1.00 | 44.26 | N |
| ATOM | 5015 | CA | VAL | A | 621 | −1.508 | −62.132 | −30.033 | 1.00 | 43.92 | C |
| ATOM | 5016 | CB | VAL | A | 621 | −1.113 | −63.573 | −29.682 | 1.00 | 44.45 | C |
| ATOM | 5017 | CG1 | VAL | A | 621 | −0.618 | −63.593 | −28.250 | 1.00 | 41.70 | C |
| ATOM | 5018 | CG2 | VAL | A | 621 | −2.286 | −64.495 | −29.957 | 1.00 | 51.65 | C |
| ATOM | 5019 | C | VAL | A | 621 | −1.595 | −61.208 | −28.812 | 1.00 | 42.77 | C |
| ATOM | 5020 | O | VAL | A | 621 | −0.695 | −60.393 | −28.620 | 1.00 | 46.48 | O |
| ATOM | 5021 | N | CYS | A | 622 | −2.659 | −61.297 | −28.011 | 1.00 | 37.06 | N |
| ATOM | 5022 | CA | CYS | A | 622 | −2.821 | −60.425 | −26.858 | 1.00 | 32.33 | C |
| ATOM | 5023 | CB | CYS | A | 622 | −4.303 | −60.107 | −26.619 | 1.00 | 36.29 | C |
| ATOM | 5024 | SG | CYS | A | 622 | −4.865 | −58.591 | −27.375 | 1.00 | 41.40 | S |
| ATOM | 5025 | C | CYS | A | 622 | −2.325 | −61.067 | −25.592 | 1.00 | 31.02 | C |
| ATOM | 5026 | O | CYS | A | 622 | −2.424 | −62.281 | −25.437 | 1.00 | 32.77 | O |
| ATOM | 5027 | N | SER | A | 623 | −1.881 | −60.243 | −24.647 | 1.00 | 29.02 | N |
| ATOM | 5028 | CA | SER | A | 623 | −1.665 | −60.700 | −23.273 | 1.00 | 28.10 | C |
| ATOM | 5029 | CB | SER | A | 623 | −0.279 | −60.278 | −22.794 | 1.00 | 26.61 | C |
| ATOM | 5030 | OG | SER | A | 623 | −0.235 | −58.882 | −22.527 | 1.00 | 26.07 | O |
| ATOM | 5031 | C | SER | A | 623 | −2.689 | −60.064 | −22.342 | 1.00 | 28.29 | C |
| ATOM | 5032 | O | SER | A | 623 | −3.436 | −59.167 | −22.725 | 1.00 | 28.98 | O |
| ATOM | 5033 | N | ALA | A | 624 | −2.700 | −60.526 | −21.110 | 1.00 | 27.76 | N |
| ATOM | 5034 | CA | ALA | A | 624 | −3.332 | −59.810 | −20.042 | 1.00 | 29.60 | C |
| ATOM | 5035 | CB | ALA | A | 624 | −3.217 | −60.609 | −18.760 | 1.00 | 27.00 | C |
| ATOM | 5036 | C | ALA | A | 624 | −2.650 | −58.461 | −19.868 | 1.00 | 34.19 | C |
| ATOM | 5037 | O | ALA | A | 624 | −1.422 | −58.318 | −20.114 | 1.00 | 40.81 | O |
| ATOM | 5038 | N | ILE | A | 625 | −3.439 | −57.468 | −19.460 | 1.00 | 33.95 | N |
| ATOM | 5039 | CA | ILE | A | 625 | −2.905 | −56.203 | −19.006 | 1.00 | 32.45 | C |
| ATOM | 5040 | CB | ILE | A | 625 | −4.024 | −55.177 | −18.753 | 1.00 | 30.13 | C |
| ATOM | 5041 | CG1 | ILE | A | 625 | −4.553 | −54.655 | −20.101 | 1.00 | 34.84 | C |
| ATOM | 5042 | CD1 | ILE | A | 625 | −5.952 | −54.045 | −20.030 | 1.00 | 31.34 | C |
| ATOM | 5043 | CG2 | ILE | A | 625 | −3.529 | −53.990 | −17.928 | 1.00 | 27.13 | C |
| ATOM | 5044 | C | ILE | A | 625 | −2.084 | −56.485 | −17.740 | 1.00 | 32.13 | C |
| ATOM | 5045 | O | ILE | A | 625 | −2.580 | −57.104 | −16.798 | 1.00 | 32.88 | O |
| ATOM | 5046 | N | HIS | A | 626 | −0.830 | −56.040 | −17.725 | 1.00 | 27.24 | N |
| ATOM | 5047 | CA | HIS | A | 626 | 0.034 | −56.259 | −16.575 | 1.00 | 26.03 | C |
| ATOM | 5048 | CB | HIS | A | 626 | 1.473 | −56.490 | −17.043 | 1.00 | 28.39 | C |
| ATOM | 5049 | CG | HIS | A | 626 | 1.706 | −57.879 | −17.531 | 1.00 | 30.28 | C |
| ATOM | 5050 | ND1 | HIS | A | 626 | 1.142 | −58.360 | −18.691 | 1.00 | 30.98 | N |
| ATOM | 5051 | CE1 | HIS | A | 626 | 1.501 | −59.619 | −18.867 | 1.00 | 31.27 | C |
| ATOM | 5052 | NE2 | HIS | A | 626 | 2.252 | −59.583 | −17.841 | 1.00 | 32.81 | N |
| ATOM | 5053 | CD2 | HIS | A | 626 | 2.393 | −58.913 | −16.987 | 1.00 | 30.97 | C |
| ATOM | 5054 | C | HIS | A | 626 | −0.014 | −55.137 | −15.575 | 1.00 | 24.55 | C |
| ATOM | 5055 | O | HIS | A | 626 | −0.097 | −53.962 | −15.943 | 1.00 | 24.63 | O |
| ATOM | 5056 | N | ASN | A | 627 | 0.044 | −55.496 | −14.298 | 1.00 | 23.08 | N |
| ATOM | 5057 | CA | ASN | A | 627 | −0.020 | −54.510 | −13.205 | 1.00 | 24.02 | C |
| ATOM | 5058 | CB | ASN | A | 627 | 1.263 | −53.699 | −13.172 | 1.00 | 22.76 | C |
| ATOM | 5059 | CG | ASN | A | 627 | 1.520 | −53.081 | −11.842 | 1.00 | 22.18 | C |
| ATOM | 5060 | OD1 | ASN | A | 627 | 1.189 | −53.657 | −10.820 | 1.00 | 23.85 | O |
| ATOM | 5061 | ND2 | ASN | A | 627 | 2.162 | −51.925 | −11.838 | 1.00 | 23.07 | N |
| ATOM | 5062 | C | ASN | A | 627 | −1.245 | −53.545 | −13.185 | 1.00 | 26.56 | C |
| ATOM | 5063 | O | ASN | A | 627 | −1.109 | −52.382 | −12.770 | 1.00 | 28.15 | O |
| ATOM | 5064 | N | PRO | A | 628 | −2.445 | −54.032 | −13.574 | 1.00 | 27.25 | N |
| ATOM | 5065 | CA | PRO | A | 628 | −3.616 | −53.159 | −13.500 | 1.00 | 26.95 | C |
| ATOM | 5066 | CB | PRO | A | 628 | −4.773 | −54.140 | −13.675 | 1.00 | 28.74 | C |
| ATOM | 5067 | CG | PRO | A | 628 | −4.238 | −55.455 | −13.241 | 1.00 | 27.58 | C |
| ATOM | 5068 | CD | PRO | A | 628 | −2.840 | −55.439 | −13.789 | 1.00 | 27.24 | C |
| ATOM | 5069 | C | PRO | A | 628 | −3.737 | −52.547 | −12.136 | 1.00 | 27.28 | C |
| ATOM | 5070 | O | PRO | A | 628 | −3.804 | −53.295 | −11.168 | 1.00 | 29.56 | O |
| ATOM | 5071 | N | THR | A | 629 | −3.760 | −51.216 | −12.022 | 1.00 | 28.21 | N |
| ATOM | 5072 | CA | THR | A | 629 | −3.984 | −50.598 | −10.700 | 1.00 | 30.64 | C |
| ATOM | 5073 | CB | THR | A | 629 | −5.185 | −51.310 | −9.983 | 1.00 | 33.47 | C |
| ATOM | 5074 | OG1 | THR | A | 629 | −6.420 | −50.716 | −10.420 | 1.00 | 35.52 | O |
| ATOM | 5075 | CG2 | THR | A | 629 | −5.119 | −51.306 | −8.471 | 1.00 | 32.79 | C |
| ATOM | 5076 | C | THR | A | 629 | −2.730 | −50.479 | −9.800 | 1.00 | 30.81 | C |
| ATOM | 5077 | O | THR | A | 629 | −2.791 | −49.934 | −8.702 | 1.00 | 30.13 | O |
| ATOM | 5078 | N | GLY | A | 630 | −1.583 | −50.963 | −10.263 | 1.00 | 32.03 | N |
| ATOM | 5079 | CA | GLY | A | 630 | −0.407 | −50.998 | 9.386 | 1.00 | 31.61 | C |
| ATOM | 5080 | C | GLY | A | 630 | −0.599 | −52.054 | −8.321 | 1.00 | 32.28 | C |
| ATOM | 5081 | O | GLY | A | 630 | −0.152 | −51.906 | −7.192 | 1.00 | 34.33 | O |
| ATOM | 5082 | N | ARG | A | 631 | −1.283 | −53.135 | −8.678 | 1.00 | 34.44 | N |
| ATOM | 5083 | CA | ARG | A | 631 | −1.624 | −54.149 | −7.704 | 1.00 | 33.67 | C |
| ATOM | 5084 | CB | ARG | A | 631 | −2.679 | −55.103 | −8.274 | 1.00 | 32.97 | C |
| ATOM | 5085 | CG | ARG | A | 631 | −2.172 | −55.955 | −9.438 | 1.00 | 29.11 | C |
| ATOM | 5086 | CD | ARG | A | 631 | −3.075 | −57.143 | −9.658 | 1.00 | 26.90 | C |
| ATOM | 5087 | NE | ARG | A | 631 | −2.746 | −57.786 | −10.910 | 1.00 | 26.60 | N |
| ATOM | 5088 | CZ | ARG | A | 631 | −3.599 | −58.487 | −11.655 | 1.00 | 29.54 | C |
| ATOM | 5089 | NH1 | ARG | A | 631 | −4.888 | −58.606 | −11.292 | 1.00 | 28.56 | N |
| ATOM | 5090 | NH2 | ARG | A | 631 | −3.171 | −59.026 | −12.798 | 1.00 | 24.76 | N |
| ATOM | 5091 | C | ARG | A | 631 | −0.371 | −54.924 | −7.284 | 1.00 | 36.83 | C |
| ATOM | 5092 | O | ARG | A | 631 | −0.317 | −55.427 | −6.173 | 1.00 | 39.92 | O |
| ATOM | 5093 | N | SER | A | 632 | 0.619 | −55.013 | −8.176 | 1.00 | 35.89 | N |
| ATOM | 5094 | CA | SER | A | 632 | 1.862 | −55.753 | −7.908 | 1.00 | 39.28 | C |
| ATOM | 5095 | CB | SER | A | 632 | 2.165 | −56.750 | −9.037 | 1.00 | 39.77 | C |
| ATOM | 5096 | OG | SER | A | 632 | 1.029 | −57.544 | −9.328 | 1.00 | 42.56 | O |
| ATOM | 5097 | C | SER | A | 632 | 3.094 | −54.875 | −7.670 | 1.00 | 38.25 | C |
| ATOM | 5098 | O | SER | A | 632 | 4.034 | −55.311 | −7.026 | 1.00 | 42.10 | O |
| ATOM | 5099 | N | TYR | A | 633 | 3.074 | −53.643 | −8.163 | 1.00 | 38.88 | N |
| ATOM | 5100 | CA | TYR | A | 633 | 4.205 | −52.721 | −8.041 | 1.00 | 37.34 | C |
| ATOM | 5101 | CB | TYR | A | 633 | 5.318 | −53.135 | −9.001 | 1.00 | 43.23 | C |
| ATOM | 5102 | CG | TYR | A | 633 | 6.089 | −52.028 | −9.673 | 1.00 | 47.49 | C |
| ATOM | 5103 | CD1 | TYR | A | 633 | 7.255 | −51.530 | −9.104 | 1.00 | 52.62 | C |
| ATOM | 5104 | CE1 | TYR | A | 633 | 7.979 | −50.524 | −9.720 | 1.00 | 55.61 | C |
| ATOM | 5105 | CZ | TYR | A | 633 | 7.539 | −49.999 | −10.928 | 1.00 | 59.81 | C |
| ATOM | 5106 | OR | TYR | A | 633 | 8.284 | −48.988 | −11.541 | 1.00 | 65.89 | O |
| ATOM | 5107 | CE2 | TYR | A | 633 | 6.378 | −50.484 | −11.515 | 1.00 | 50.57 | C |
| ATOM | 5108 | CD2 | TYR | A | 633 | 5.682 | −51.509 | −10.896 | 1.00 | 43.26 | C |
| ATOM | 5109 | C | TYR | A | 633 | 3.766 | −51.289 | −8.305 | 1.00 | 37.34 | C |
| ATOM | 5110 | O | TYR | A | 633 | 3.054 | −51.022 | −9.291 | 1.00 | 37.53 | O |
| ATOM | 5111 | N | THR | A | 634 | 4.174 | −50.399 | −7.394 | 1.00 | 35.61 | N |
| ATOM | 5112 | CA | THR | A | 634 | 4.106 | −48.941 | −7.534 | 1.00 | 35.41 | C |
| ATOM | 5113 | CB | THR | A | 634 | 2.999 | −48.362 | −6.659 | 1.00 | 33.01 | C |
| ATOM | 5114 | OG1 | THR | A | 634 | 3.226 | −48.788 | −5.308 | 1.00 | 49.65 | O |
| ATOM | 5115 | CG2 | THR | A | 634 | 1.608 | −48.829 | −7.121 | 1.00 | 37.53 | C |
| ATOM | 5116 | C | THR | A | 634 | 5.408 | −48.302 | −7.008 | 1.00 | 37.80 | C |
| ATOM | 5117 | O | THR | A | 634 | 6.196 | −48.933 | −6.291 | 1.00 | 34.42 | O |
| ATOM | 5118 | N | ILE | A | 635 | 5.604 | −47.034 | −7.343 | 1.00 | 37.14 | N |
| ATOM | 5119 | CA | ILE | A | 635 | 6.688 | −46.232 | −6.801 | 1.00 | 3.54 | C |
| ATOM | 5120 | CE | ILE | A | 635 | 7.487 | −45.585 | −7.941 | 1.00 | 35.15 | C |
| ATOM | 5121 | CG1 | ILE | A | 635 | 7.984 | −46.651 | −8.906 | 1.00 | 37.42 | C |
| ATOM | 5122 | CD1 | ILE | A | 635 | 7.760 | −46.254 | −10.356 | 1.00 | 42.58 | C |
| ATOM | 5123 | CG2 | ILE | A | 635 | 8.667 | −44.805 | −7.414 | 1.00 | 34.70 | C |
| ATOM | 5124 | C | ILE | A | 635 | 6.039 | −45.157 | −5.942 | 1.00 | 35.55 | C |
| ATOM | 5125 | O | ILE | A | 635 | 5.157 | −44.433 | −6.413 | 1.00 | 35.81 | O |
| ATOM | 5126 | N | GLY | A | 636 | 6.447 | −45.069 | −4.679 | 1.00 | 33.67 | N |
| ATOM | 5127 | CA | GLY | A | 636 | 5.824 | −44.133 | −3.767 | 1.00 | 32.06 | C |
| ATOM | 5128 | C | GLY | A | 636 | 6.189 | −42.670 | −3.990 | 1.00 | 35.89 | C |
| ATOM | 5129 | O | GLY | A | 636 | 7.386 | −42.321 | −4.135 | 1.00 | 33.11 | O |
| ATOM | 5130 | N | LEU | A | 637 | 5.159 | −41.815 | −3.983 | 1.00 | 34.23 | N |
| ATOM | 5131 | CA | LEU | A | 637 | 5.310 | −40.348 | −3.984 | 1.00 | 37.38 | C |
| ATOM | 5132 | CB | LEU | A | 637 | 4.096 | −39.670 | −4.629 | 1.00 | 40.29 | C |
| ATOM | 5133 | CG | LEU | A | 637 | 4.205 | −39.649 | −6.143 | 1.00 | 38.91 | C |
| ATOM | 5134 | CD1 | LEU | A | 637 | 2.946 | −39.042 | −6.687 | 1.00 | 38.94 | C |
| ATOM | 5135 | CD2 | LEU | A | 637 | 5.440 | −38.870 | −6.589 | 1.00 | 36.40 | C |
| ATOM | 5136 | C | LEU | A | 637 | 5.541 | −39.729 | −2.606 | 1.00 | 38.18 | C |
| ATOM | 5137 | O | LEU | A | 637 | 5.693 | −40.447 | −1.617 | 1.00 | 42.38 | O |
| ATOM | 5138 | N | LEU | B | 19 | 45.135 | −123.802 | −20.014 | 1.00 | 122.04 | N |
| ATOM | 5139 | CA | LEU | B | 19 | 45.158 | −122.321 | −20.781 | 1.00 | 125.22 | C |
| ATOM | 5140 | CB | LEU | B | 19 | 44.740 | −121.324 | −19.897 | 1.00 | 122.10 | C |
| ATOM | 5141 | CG | LEU | B | 19 | 45.477 | −120.970 | −18.590 | 1.00 | 119.63 | C |
| ATOM | 5142 | CD1 | LEU | B | 19 | 45.526 | −119.459 | −18.391 | 1.00 | 108.38 | C |
| ATOM | 5143 | CD2 | LEU | B | 19 | 44.889 | −121.662 | −17.360 | 1.00 | 113.33 | C |
| ATOM | 5144 | C | LEU | B | 19 | 46.523 | −122.281 | −21.453 | 1.00 | 128.86 | C |
| ATOM | 5145 | O | LEU | B | 19 | 47.563 | −122.341 | −20.789 | 1.00 | 126.81 | O |
| ATOM | 5146 | N | PRO | B | 20 | 46.519 | −122.034 | −22.782 | 1.00 | 131.42 | N |
| ATOM | 5147 | CA | PRO | B | 20 | 47.705 | −121.697 | −23.594 | 1.00 | 133.21 | C |
| ATOM | 5148 | CB | PRO | B | 20 | 47.107 | −121.460 | −24.987 | 1.00 | 126.10 | C |
| ATOM | 5149 | CG | PRO | B | 20 | 45.903 | −122.330 | −25.015 | 1.00 | 122.61 | C |
| ATOM | 5150 | CD | PRO | B | 20 | 45.332 | −122.262 | −23.629 | 1.00 | 126.00 | C |
| ATOM | 5151 | C | PRO | B | 20 | 48.466 | −120.440 | −23.122 | 1.00 | 135.70 | C |
| ATOM | 5152 | O | PRO | B | 20 | 47.963 | −119.718 | −22.253 | 1.00 | 129.20 | O |
| ATOM | 5153 | N | PRO | B | 21 | 49.677 | −120.186 | −23.681 | 1.00 | 142.28 | N |
| ATOM | 5154 | CA | PRO | B | 21 | 50.398 | −118.937 | −23.389 | 1.00 | 142.60 | C |
| ATOM | 5155 | CB | PRO | B | 21 | 51.748 | −119.133 | −24.096 | 1.00 | 137.99 | C |
| ATOM | 5156 | CG | PRO | B | 21 | 51.482 | −120.143 | −25.160 | 1.00 | 137.69 | C |
| ATOM | 5157 | CD | PRO | B | 21 | 50.472 | −121.074 | −24.555 | 1.00 | 142.20 | C |
| ATOM | 5158 | C | PRO | B | 21 | 49.673 | −117.215 | −23.963 | 1.00 | 144.51 | C |
| ATOM | 5159 | O | PRO | B | 21 | 48.983 | −117.838 | −24.984 | 1.00 | 146.22 | O |
| ATOM | 5160 | N | ASN | B | 22 | 49.822 | −116.561 | −23.301 | 1.00 | 137.84 | N |
| ATOM | 5161 | CA | ASN | B | 22 | 49.198 | −115.289 | −23.725 | 1.00 | 132.31 | C |
| ATOM | 5162 | CB | ASN | B | 22 | 49.565 | −114.969 | −25.194 | 1.00 | 128.60 | C |
| ATOM | 5163 | CG | ASN | B | 22 | 48.894 | −113.712 | −25.724 | 1.00 | 118.32 | C |
| ATOM | 5164 | OD1 | ASN | B | 22 | 48.950 | −112.655 | −25.099 | 1.00 | 116.11 | O |
| ATOM | 5165 | ND2 | ASN | B | 22 | 48.268 | −113.822 | −26.896 | 1.00 | 113.32 | N |
| ATOM | 5166 | C | ASN | B | 32 | 47.674 | −115.223 | −23.479 | 1.00 | 132.28 | C |
| ATOM | 5167 | O | ASN | B | 32 | 47.079 | −114.138 | −23.467 | 1.00 | 122.50 | O |
| ATOM | 5168 | N | ALA | B | 23 | 47.059 | −116.387 | 23.268 | 1.00 | 131.82 | N |
| ATOM | 5169 | CA | ALA | B | 23 | 45.619 | −116.492 | −23.036 | 1.00 | 130.48 | C |
| ATOM | 5170 | CB | ALA | B | 23 | 45.158 | −117.933 | −23.215 | 1.00 | 131.09 | C |
| ATOM | 5171 | C | ALA | B | 23 | 45.233 | −115.981 | −21.650 | 1.00 | 132.56 | C |
| ATOM | 5172 | O | ALA | B | 23 | 45.935 | −116.256 | −20.672 | 1.00 | 134.71 | O |
| ATOM | 5173 | N | PRO | B | 24 | 44.122 | −115.222 | −21.564 | 1.00 | 131.61 | N |
| ATOM | 5174 | CA | PRO | B | 24 | 43.585 | −114.810 | −20.265 | 1.00 | 130.05 | C |
| ATOM | 5175 | CB | PRO | B | 24 | 42.449 | −113.847 | −20.637 | 1.00 | 129.79 | C |
| ATOM | 5176 | CG | PRO | B | 24 | 42.745 | −113.416 | −22.033 | 1.00 | 128.82 | C |
| ATOM | 5177 | CD | PRO | B | 24 | 43.380 | −114.609 | −22.679 | 1.00 | 129.68 | C |
| ATOM | 5178 | C | PRO | B | 24 | 43.037 | −116.008 | −19.484 | 1.00 | 127.75 | C |
| ATOM | 5179 | O | PRO | B | 24 | 42.530 | −116.965 | −20.084 | 1.00 | 118.40 | O |
| ATOM | 5180 | N | ALA | B | 25 | 43.151 | −115.947 | −18.158 | 1.00 | 126.80 | N |
| ATOM | 5181 | CA | ALA | B | 25 | 42.723 | −117.037 | −17.276 | 1.00 | 126.19 | C |
| ATOM | 5182 | CB | ALA | B | 25 | 43.270 | −116.826 | −15.868 | 1.00 | 118.51 | C |
| ATOM | 5183 | C | ALA | B | 25 | 41.199 | −117.390 | −17.251 | 1.00 | 121.78 | C |
| ATOM | 5181 | O | ALA | B | 25 | 40.676 | −118.295 | −17.073 | 1.00 | 123.15 | O |
| ATOM | 5185 | N | CYS | B | 26 | 40.501 | −116.073 | −17.443 | 1.00 | 116.91 | N |
| ATOM | 5186 | CA | CYS | B | 26 | 39.046 | −116.035 | −17.390 | 1.00 | 104.49 | C |
| ATOM | 5187 | CB | CYS | B | 26 | 38.590 | −115.288 | −16.137 | 1.00 | 104.37 | C |
| ATOM | 5188 | SG | CYS | B | 26 | 36.797 | −115.164 | −15.991 | 1.00 | 112.51 | S |
| ATOM | 5189 | C | CYS | B | 26 | 38.460 | −115.383 | −18.641 | 1.00 | 94.78 | C |
| ATOM | 5190 | O | CYS | B | 26 | 39.060 | −114.471 | −19.222 | 1.00 | 93.14 | O |
| ATOM | 5191 | N | MET | B | 27 | 37.283 | −115.851 | −19.048 | 1.00 | 83.06 | N |
| ATOM | 5192 | CA | MET | B | 27 | 36.618 | −115.312 | −20.231 | 1.00 | 75.90 | C |
| ATOM | 5193 | CB | MET | B | 27 | 36.148 | −116.436 | −21.158 | 1.00 | 74.40 | C |
| ATOM | 5194 | CG | MET | B | 27 | 35.419 | −115.934 | −22.395 | 1.00 | 73.20 | C |
| ATOM | 5195 | SD | MET | B | 27 | 35.082 | −117.224 | −23.593 | 1.00 | 77.25 | S |
| ATOM | 5196 | CE | MET | B | 27 | 33.710 | −118.043 | −22.820 | 1.00 | 74.69 | C |
| ATOM | 5197 | C | MET | B | 27 | 35.435 | −114.409 | −19.891 | 1.00 | 71.76 | C |
| ATOM | 5198 | O | MET | B | 27 | 34.529 | −114.821 | −19.160 | 1.00 | 70.50 | O |
| ATOM | 5199 | N | GLU | B | 28 | 35.456 | −113.195 | −20.456 | 1.00 | 69.16 | N |
| ATOM | 5200 | CA | GLU | B | 28 | 34.360 | −112.205 | −20.396 | 1.00 | 62.12 | C |
| ATOM | 5201 | CB | GLU | B | 28 | 34.543 | −111.188 | −71.522 | 1.00 | 57.61 | C |
| ATOM | 5202 | CG | GLU | B | 28 | 35.469 | −110.034 | −21.192 | 1.00 | 60.60 | C |
| ATOM | 5203 | CD | GLU | B | 28 | 36.944 | −110.373 | −21.291 | 1.00 | 65.36 | C |
| ATOM | 5204 | OE1 | GLU | B | 28 | 37.290 | −111.407 | −21.892 | 1.00 | 71.13 | O |
| ATOM | 5205 | OE2 | GLU | B | 28 | 37.765 | −109.598 | −20.764 | 1.00 | 65.72 | O |
| ATOM | 5206 | C | GLU | B | 28 | 32.963 | −112.835 | −20.488 | 1.00 | 56.57 | C |
| ATOM | 5207 | O | GLU | B | 28 | 32.720 | −113.688 | −21.332 | 1.00 | 55.25 | O |
| ATOM | 5208 | N | ARG | B | 29 | 32.058 | −112.420 | −19.610 | 1.00 | 56.30 | N |
| ATOM | 5209 | CA | ARG | B | 29 | 30.757 | −113.085 | −19.467 | 1.00 | 55.73 | C |
| ATOM | 5210 | CB | ARG | B | 29 | 30.023 | −112.555 | −18.230 | 1.00 | 60.17 | C |
| ATOM | 5211 | CG | ARG | B | 29 | 28.526 | −112.846 | −18.177 | 1.00 | 65.14 | C |
| ATOM | 5212 | CD | ARG | B | 29 | 28.190 | −114.100 | −17.383 | 1.00 | 72.48 | C |
| ATOM | 5213 | NE | ARG | B | 29 | 26.744 | −114.314 | −17.320 | 1.00 | 76.48 | N |
| ATOM | 5214 | CZ | ARG | B | 29 | 26.156 | −115.400 | −16.816 | 1.00 | 78.40 | C |
| ATOM | 5215 | NH1 | ARG | B | 29 | 24.823 | −115.494 | −16.813 | 1.00 | 74.25 | N |
| ATOM | 5216 | NH2 | ARG | B | 29 | 26.892 | −116.393 | −16.321 | 1.00 | 69.36 | N |
| ATOM | 5217 | C | ARG | B | 29 | 29.866 | −112.995 | −20.710 | 1.00 | 53.36 | C |
| ATOM | 5218 | O | ARG | B | 29 | 29.227 | −113.987 | −21.094 | 1.00 | 58.60 | O |
| ATOM | 5219 | N | GLN | B | 30 | 29.831 | −111.828 | −21.350 | 1.00 | 52.03 | N |
| ATOM | 5220 | CA | GLN | B | 30 | 28.881 | −111.607 | −22.445 | 1.00 | 52.43 | C |
| ATOM | 5221 | CB | GLN | B | 30 | 28.360 | −110.165 | −22.483 | 1.00 | 52.12 | C |
| ATOM | 5222 | CG | GLN | B | 30 | 27.459 | −109.810 | −21.306 | 1.00 | 50.58 | C |
| ATOM | 5223 | CD | GLN | B | 30 | 28.233 | −109.562 | −20.013 | 1.00 | 47.46 | C |
| ATOM | 5224 | OE1 | GLN | B | 30 | 29.379 | −109.075 | −20.021 | 1.00 | 47.64 | O |
| ATOM | 5225 | NE2 | GLN | B | 30 | 27.606 | −109.886 | −18.896 | 1.00 | 41.54 | N |
| ATOM | 5226 | C | GLN | B | 30 | 29.437 | −112.047 | −23.795 | 1.00 | 53.91 | C |
| ATOM | 5227 | O | GLN | B | 30 | 30.243 | −111.339 | −24.418 | 1.00 | 48.03 | O |
| ATOM | 5228 | N | LEU | B | 31 | 28.988 | −113.224 | −24.230 | 1.00 | 52.77 | N |
| ATOM | 5229 | CA | LEU | B | 31 | 29.369 | −113.765 | −25.522 | 1.00 | 54.99 | C |
| ATOM | 5230 | CB | LEU | B | 31 | 29.130 | −115.282 | −25.562 | 1.00 | 54.27 | C |
| ATOM | 5231 | CG | LEU | B | 31 | 29.895 | −116.112 | −24.502 | 1.00 | 51.55 | C |
| ATOM | 5232 | CD1 | LEU | B | 31 | 29.537 | −117.587 | −24.557 | 1.00 | 46.64 | C |
| ATOM | 5233 | CD2 | LEU | B | 31 | 31.401 | −115.919 | −24.634 | 1.00 | 49.67 | C |
| ATOM | 5234 | C | LEU | B | 31 | 28.625 | −113.046 | −26.642 | 1.00 | 55.20 | C |
| ATOM | 5235 | O | LEU | B | 31 | 27.392 | −113.026 | −26.659 | 1.00 | 58.64 | O |
| ATOM | 5236 | N | GLU | B | 32 | 29.388 | −112.458 | −27.563 | 1.00 | 55.75 | N |
| ATOM | 5237 | CA | GLU | B | 32 | 28.847 | −111.646 | −28.654 | 1.00 | 55.00 | C |
| ATOM | 5238 | CB | GLU | B | 32 | 29.635 | −110.337 | −28.770 | 1.00 | 57.15 | C |
| ATOM | 5239 | CG | GLU | B | 32 | 28.814 | −109.171 | −29.337 | 1.00 | 64.38 | C |
| ATOM | 5240 | CD | GLU | B | 32 | 27.472 | −108.983 | −28.629 | 1.00 | 62.78 | C |
| ATOM | 5241 | OE1 | GLU | B | 32 | 27.435 | −109.026 | −27.371 | 1.00 | 61.50 | O |
| ATOM | 5242 | OE2 | GLU | B | 32 | 26.453 | −108.806 | −29.332 | 1.00 | 57.80 | O |
| ATOM | 5243 | C | GLU | B | 32 | 28.808 | −112.330 | −30.026 | 1.00 | 55.64 | C |
| ATOM | 5244 | O | GLU | B | 32 | 27.918 | −112.045 | −30.841 | 1.00 | 59.67 | O |
| ATOM | 5245 | N | ALA | B | 33 | 29.774 | −113.215 | −30.284 | 1.00 | 55.92 | N |
| ATOM | 5246 | CA | ALA | B | 33 | 29.984 | −113.777 | −31.623 | 1.00 | 55.87 | C |
| ATOM | 5247 | CB | ALA | B | 33 | 30.914 | −112.878 | −32.431 | 1.00 | 54.95 | C |
| ATOM | 5248 | C | ALA | B | 33 | 30.512 | −115.206 | −31.618 | 1.00 | 57.12 | C |
| ATOM | 5249 | O | ALA | B | 33 | 31.312 | −115.588 | −30.756 | 1.00 | 53.36 | O |
| ATOM | 5250 | N | ALA | B | 34 | 30.061 | −115.979 | −32.607 | 1.00 | 62.10 | N |
| ATOM | 5251 | CA | ALA | B | 34 | 30.468 | −117.174 | −32.806 | 1.00 | 57.99 | C |
| ATOM | 5252 | CB | ALA | B | 34 | 29.505 | −118.311 | −32.101 | 1.00 | 52.57 | C |
| ATOM | 5253 | C | ALA | B | 34 | 30.518 | −117.695 | −34.294 | 1.00 | 58.37 | C |
| ATOM | 5254 | O | ALA | B | 34 | 29.490 | −117.630 | −34.973 | 1.00 | 55.15 | O |
| ATOM | 5255 | N | ARG | B | 35 | 31.709 | −118.044 | −34.789 | 1.00 | 62.02 | N |
| ATOM | 5256 | CA | ARG | B | 35 | 31.933 | −118.310 | −36.226 | 1.00 | 62.92 | C |
| ATOM | 5257 | CB | ARG | B | 35 | 32.549 | −117.088 | −36.936 | 1.00 | 64.58 | C |
| ATOM | 5258 | CG | ARG | B | 35 | 31.833 | −115.760 | −36.741 | 1.00 | 67.32 | C |
| ATOM | 5259 | CD | ARG | B | 35 | 30.473 | −115.697 | −37.424 | 1.00 | 66.64 | C |
| ATOM | 5260 | NE | ARG | B | 35 | 29.674 | −114.627 | −36.838 | 1.00 | 68.37 | N |
| ATOM | 5261 | CZ | ARG | B | 35 | 29.818 | −113.333 | −37.120 | 1.00 | 70.62 | C |
| ATOM | 5262 | NH1 | ARG | B | 35 | 30.719 | −112.922 | −38.006 | 1.00 | 63.99 | N |
| ATOM | 5263 | NH2 | ARG | B | 35 | 29.048 | −112.444 | −36.511 | 1.00 | 75.51 | N |
| ATOM | 5264 | C | ARG | B | 35 | 32.828 | −119.521 | −36.503 | 1.00 | 61.65 | C |
| ATOM | 5265 | O | ARG | B | 35 | 34.024 | −119.519 | −36.160 | 1.00 | 57.20 | O |
| ATOM | 5266 | N | TYR | B | 36 | 32.257 | −120.535 | −37.153 | 1.00 | 59.56 | N |
| ATOM | 5267 | CA | TYR | B | 36 | 33.037 | −121.691 | −37.613 | 1.00 | 60.10 | C |
| ATOM | 5268 | CB | TYR | B | 36 | 32.125 | −122.847 | −38.000 | 1.00 | 58.85 | C |
| ATOM | 5269 | CG | TYR | B | 36 | 31.583 | −123.663 | −36.846 | 1.00 | 60.85 | C |
| ATOM | 5270 | CD1 | TYR | B | 36 | 32.430 | −124.410 | −36.033 | 1.00 | 59.59 | C |
| ATOM | 5271 | CE1 | TYR | B | 36 | 31.929 | −125.170 | −34.987 | 1.00 | 62.51 | C |
| ATOM | 5272 | CZ | TYR | B | 36 | 30.563 | −125.197 | −34.746 | 1.00 | 59.97 | C |
| ATOM | 5273 | OH | TYR | B | 36 | 30.061 | −125.945 | −33.707 | 1.00 | 56.36 | O |
| ATOM | 5274 | CE2 | TYR | B | 36 | 29.701 | −124.474 | −35.551 | 1.00 | 63.47 | C |
| ATOM | 5275 | CD2 | TYR | B | 36 | 30.211 | −123.719 | −36.596 | 1.00 | 62.54 | C |
| ATOM | 5276 | C | TYR | B | 36 | 33.898 | −121.356 | −38.824 | 1.00 | 64.81 | C |
| ATOM | 5277 | O | TYR | B | 36 | 33.386 | −120.859 | −39.832 | 1.00 | 63.35 | O |
| ATOM | 5278 | N | ARG | B | 37 | 35.200 | −121.636 | −38.715 | 1.00 | 64.78 | N |
| ATOM | 5279 | CA | ARG | B | 37 | 36.118 | −121.683 | −39.866 | 1.00 | 64.49 | C |
| ATOM | 5280 | CB | ARG | B | 37 | 37.555 | −121.731 | −39.353 | 1.00 | 59.53 | C |
| ATOM | 5281 | CG | ARG | B | 37 | 38.627 | −121.553 | −40.401 | 1.00 | 57.03 | C |
| ATOM | 5282 | CD | ARG | B | 37 | 39.962 | −121.193 | −39.728 | 1.00 | 60.91 | C |
| ATOM | 5283 | NE | ARG | B | 37 | 40.495 | −122.450 | −39.000 | 1.00 | 59.93 | N |
| ATOM | 5284 | CZ | ARG | B | 37 | 41.706 | −122.496 | −38.443 | 1.00 | 60.38 | C |
| ATOM | 5285 | NH1 | ARG | B | 37 | 42.526 | −121.448 | −38.521 | 1.00 | 56.88 | N |
| ATOM | 5286 | NH2 | ARG | B | 37 | 42.104 | −123.593 | −37.805 | 1.00 | 60.97 | N |
| ATOM | 5287 | C | ARG | B | 37 | 35.809 | −122.931 | −40.707 | 1.00 | 66.20 | C |
| ATOM | 5288 | O | ARG | B | 37 | 35.160 | −123.857 | −40.215 | 1.00 | 69.97 | O |
| ATOM | 5289 | N | SER | B | 38 | 36.260 | −122.966 | −41.963 | 1.00 | 73.01 | N |
| ATOM | 5290 | CA | SER | B | 38 | 35.964 | −124.112 | −42.853 | 1.00 | 74.02 | C |
| ATOM | 5291 | CB | SER | B | 38 | 36.388 | −123.856 | −44.308 | 1.00 | 72.12 | C |
| ATOM | 5292 | OG | SER | B | 38 | 37.533 | −123.825 | −44.384 | 1.00 | 81.51 | O |
| ATOM | 5293 | C | SER | B | 38 | 36.521 | −125.437 | −42.328 | 1.00 | 76.31 | C |
| ATOM | 5294 | O | SER | B | 38 | 35.884 | −126.485 | −42.490 | 1.00 | 76.52 | O |
| ATOM | 5295 | N | ASP | B | 39 | 37.676 | −125.583 | −41.663 | 1.00 | 77.98 | N |
| ATOM | 5296 | CA | ASP | B | 39 | 38.256 | −126.574 | −41.020 | 1.00 | 79.09 | C |
| ATOM | 5297 | CB | ASP | B | 39 | 39.723 | −126.334 | −40.630 | 1.00 | 76.84 | C |
| ATOM | 5298 | CG | ASP | B | 39 | 39.872 | −125.623 | −39.300 | 1.00 | 79.14 | C |
| ATOM | 5299 | OD1 | ASP | B | 39 | 39.002 | −124.794 | −38.958 | 1.00 | 78.59 | O |
| ATOM | 5300 | OD2 | ASP | B | 39 | 40.870 | −125.888 | −38.595 | 1.00 | 77.33 | O |
| ATOM | 5301 | C | ASP | B | 39 | 37.446 | −127.699 | −39.818 | 1.00 | 80.70 | C |
| ATOM | 5302 | O | ASP | B | 39 | 37.704 | −128.198 | −39.325 | 1.00 | 87.43 | O |
| ATOM | 5303 | N | GLY | B | 40 | 36.486 | −126.306 | −39.341 | 1.00 | 80.72 | N |
| ATOM | 5304 | CA | GLY | B | 40 | 35.549 | −126.742 | −38.289 | 1.00 | 70.56 | C |
| ATOM | 5305 | C | GLY | B | 40 | 35.811 | −126.231 | −36.880 | 1.00 | 64.86 | C |
| ATOM | 5306 | O | GLY | B | 40 | 35.085 | −126.591 | −35.945 | 1.00 | 59.32 | O |
| ATOM | 5307 | N | ALA | B | 41 | 36.855 | −125.412 | −36.731 | 1.00 | 58.55 | N |
| ATOM | 5308 | CA | ALA | B | 41 | 37.219 | −124.790 | −35.455 | 1.00 | 60.17 | C |
| ATOM | 5309 | CB | ALA | B | 41 | 38.698 | −124.429 | −35.457 | 1.00 | 56.92 | C |
| ATOM | 5310 | C | ALA | B | 41 | 36.358 | −123.548 | −35.127 | 1.00 | 63.32 | C |
| ATOM | 5311 | O | ALA | B | 41 | 35.912 | −122.832 | −36.037 | 1.00 | 64.27 | O |
| ATOM | 5312 | N | LEU | B | 42 | 36.154 | −123.292 | −33.829 | 1.00 | 63.15 | N |
| ATOM | 5313 | CA | LEU | B | 42 | 35.267 | −122.216 | −33.351 | 1.00 | 60.47 | C |
| ATOM | 5314 | CB | LEU | B | 42 | 34.408 | −122.703 | −32.189 | 1.00 | 61.52 | C |
| ATOM | 5315 | CG | LEU | B | 42 | 33.106 | −121.947 | −31.913 | 1.00 | 62.54 | C |
| ATOM | 5316 | CD1 | LEU | B | 42 | 32.437 | −121.435 | −33.183 | 1.00 | 59.88 | C |
| ATOM | 5317 | CD2 | LEU | B | 42 | 32.144 | −122.850 | −31.149 | 1.00 | 64.25 | C |
| ATOM | 5318 | C | LEU | B | 42 | 35.965 | −120.928 | −32.928 | 1.00 | 60.17 | C |
| ATOM | 5319 | O | LEU | B | 42 | 36.839 | −120.936 | −32.048 | 1.00 | 58.84 | O |
| ATOM | 5320 | N | LEU | B | 43 | 35.560 | −119.832 | −33.573 | 1.00 | 56.67 | N |
| ATOM | 5321 | CA | LEU | B | 43 | 35.961 | −118.474 | −33.200 | 1.00 | 57.88 | C |
| ATOM | 5322 | CB | LEU | B | 43 | 36.156 | −117.599 | −34.444 | 1.00 | 55.28 | C |
| ATOM | 5323 | CG | LEU | B | 43 | 36.432 | −116.101 | −34.236 | 1.00 | 54.55 | C |
| ATOM | 5324 | CD1 | LEU | B | 43 | 37.652 | −115.811 | −33.360 | 1.00 | 54.33 | C |
| ATOM | 5325 | CD2 | LEU | B | 43 | 36.559 | −115.401 | −35.577 | 1.00 | 51.41 | C |
| ATOM | 5326 | C | LEU | B | 43 | 34.904 | −117.836 | −32.308 | 1.00 | 58.21 | C |
| ATOM | 5327 | O | LEU | B | 43 | 33.735 | −117.721 | −32.710 | 1.00 | 60.87 | O |
| ATOM | 5328 | N | LEU | B | 44 | 35.325 | −117.428 | −31.110 | 1.00 | 57.56 | N |
| ATOM | 5329 | CA | LEU | B | 44 | 34.469 | −116.713 | −30.150 | 1.00 | 55.53 | C |
| ATOM | 5330 | CB | LEU | B | 44 | 34.402 | −117.435 | −28.803 | 1.00 | 53.04 | C |
| ATOM | 5331 | CG | LEU | B | 44 | 33.863 | −118.855 | −28.725 | 1.00 | 58.81 | C |
| ATOM | 5332 | CD1 | LEU | B | 44 | 33.851 | −119.306 | −27.268 | 1.00 | 62.93 | C |
| ATOM | 5333 | CD2 | LEU | B | 44 | 32.478 | −118.983 | −29.336 | 1.00 | 55.87 | C |
| ATOM | 5334 | C | LEU | B | 44 | 34.977 | −115.305 | −29.890 | 1.00 | 53.33 | C |
| ATOM | 5335 | O | LEU | B | 44 | 36.178 | −115.102 | −29.669 | 1.00 | 52.60 | O |
| ATOM | 5336 | N | GLY | B | 45 | 34.052 | −114.343 | −29.915 | 1.00 | 54.02 | N |
| ATOM | 5337 | CA | GLY | B | 45 | 34.292 | −112.988 | −29.395 | 1.00 | 47.39 | C |
| ATOM | 5338 | C | GLY | B | 45 | 33.418 | −112.764 | −28.177 | 1.00 | 46.88 | C |
| ATOM | 5339 | O | GLY | B | 45 | 32.250 | −113.200 | −28.149 | 1.00 | 45.83 | O |
| ATOM | 5340 | N | ALA | B | 46 | 33.979 | −112.107 | −27.164 | 1.00 | 45.98 | N |
| ATOM | 5341 | CA | ALA | B | 46 | 33.248 | −111.811 | −25.926 | 1.00 | 46.29 | C |
| ATOM | 5342 | CB | ALA | B | 46 | 33.436 | −112.920 | −24.896 | 1.00 | 46.04 | C |
| ATOM | 5343 | C | ALA | B | 46 | 33.649 | −110.464 | −25.324 | 1.00 | 48.53 | C |
| ATOM | 5344 | O | ALA | B | 46 | 34.825 | −110.049 | −25.414 | 1.00 | 44.72 | O |
| ATOM | 5345 | N | SER | B | 47 | 32.660 | −109.804 | −24.713 | 1.00 | 43.57 | N |
| ATOM | 5346 | CA | SER | B | 47 | 32.850 | −106.517 | −24.041 | 1.00 | 44.03 | C |
| ATOM | 5347 | CB | SER | B | 47 | 31.949 | −107.415 | −24.640 | 1.00 | 44.79 | C |
| ATOM | 5348 | OG | SER | B | 47 | 32.085 | −107.258 | −26.048 | 1.00 | 46.51 | O |
| ATOM | 5349 | C | SER | B | 47 | 32.519 | −108.635 | −22.570 | 1.00 | 43.23 | C |
| ATOM | 5350 | O | SER | B | 47 | 31.747 | −109.508 | −22.145 | 1.00 | 43.50 | O |
| ATOM | 5351 | N | SER | B | 48 | 33.112 | −107.743 | −21.793 | 1.00 | 46.73 | N |
| ATOM | 5352 | CA | SER | B | 48 | 32.694 | −107.509 | −20.413 | 1.00 | 49.82 | C |
| ATOM | 5353 | CB | SER | B | 48 | 33.922 | −107.320 | −19.523 | 1.00 | 51.45 | C |
| ATOM | 5354 | OG | SER | B | 48 | 33.566 | −106.762 | −18.280 | 1.00 | 57.12 | O |
| ATOM | 5355 | C | SER | B | 48 | 31.812 | −106.248 | −20.400 | 1.00 | 47.36 | C |
| ATOM | 5356 | O | SER | B | 48 | 32.320 | −105.124 | −20.480 | 1.00 | 33.79 | O |
| ATOM | 5357 | N | LEU | B | 49 | 30.498 | −106.453 | −20.325 | 1.00 | 46.95 | N |
| ATOM | 5358 | CA | LEU | B | 49 | 29.533 | −105.354 | −20.374 | 1.00 | 48.39 | C |
| ATOM | 5359 | CB | LEU | B | 49 | 28.279 | −105.786 | −21.148 | 1.00 | 48.09 | C |
| ATOM | 5360 | CG | LEU | B | 49 | 28.494 | −106.173 | −22.615 | 1.00 | 47.28 | C |
| ATOM | 5361 | CD1 | LEU | B | 49 | 27.159 | −106.417 | −23.291 | 1.00 | 42.93 | C |
| ATOM | 5362 | CD2 | LEU | B | 49 | 29.330 | −105.151 | −23.384 | 1.00 | 43.35 | C |
| ATOM | 5363 | C | LEU | B | 49 | 29.119 | −104.795 | −19.002 | 1.00 | 53.14 | C |
| ATOM | 5364 | O | LEU | B | 49 | 28.250 | −103.903 | −18.921 | 1.00 | 54.59 | O |
| ATOM | 5365 | N | SER | B | 50 | 29.727 | −105.320 | −17.935 | 1.00 | 48.18 | N |
| ATOM | 5366 | CA | SER | B | 50 | 29.417 | −104.899 | −16.578 | 1.00 | 47.02 | C |
| ATOM | 5367 | CB | SER | B | 50 | 28.179 | −105.642 | −16.083 | 1.00 | 46.27 | C |
| ATOM | 5368 | OG | SER | B | 50 | 27.602 | −104.949 | −15.003 | 1.00 | 41.36 | O |
| ATOM | 5369 | C | SER | B | 50 | 30.608 | −105.119 | −15.645 | 1.00 | 51.44 | C |
| ATOM | 5370 | O | SER | B | 50 | 31.643 | −105.635 | −16.074 | 1.00 | 59.56 | O |
| ATOM | 5371 | N | GLY | B | 51 | 30.483 | −104.714 | −14.379 | 1.00 | 51.37 | N |
| ATOM | 5372 | CA | GLY | B | 51 | 31.595 | −104.803 | −13.435 | 1.00 | 45.64 | C |
| ATOM | 5373 | C | GLY | B | 51 | 32.710 | −103.810 | −13.746 | 1.00 | 49.05 | C |
| ATOM | 5374 | O | GLY | B | 51 | 32.555 | −102.938 | −14.597 | 1.00 | 49.85 | O |
| ATOM | 5375 | N | ARG | B | 52 | 33.845 | −103.957 | −13.062 | 1.00 | 48.23 | N |
| ATOM | 5376 | CA | ARG | B | 52 | 34.892 | −102.947 | −13.058 | 1.00 | 43.11 | C |
| ATOM | 5377 | CB | ARG | B | 52 | 36.012 | −103.327 | −12.074 | 1.00 | 50.58 | C |
| ATOM | 5378 | CG | ARG | B | 52 | 37.191 | −102.346 | −12.051 | 1.00 | 50.59 | C |
| ATOM | 5379 | CD | ARG | B | 52 | 38.425 | −102.968 | −11.405 | 1.00 | 58.68 | C |
| ATOM | 5380 | NE | ARG | B | 52 | 38.276 | −103.133 | −9.957 | 1.00 | 63.00 | N |
| ATOM | 5381 | CZ | ARG | B | 52 | 38.892 | −102.389 | −9.036 | 1.00 | 64.43 | C |
| ATOM | 5382 | NH1 | ARG | B | 52 | 39.731 | −101.428 | −9.407 | 1.00 | 66.95 | N |
| ATOM | 5383 | NH2 | ARG | B | 52 | 38.673 | −102.609 | −7.739 | 1.00 | 63.06 | N |
| ATOM | 5384 | C | ARG | B | 52 | 35.486 | −102.666 | −14.438 | 1.00 | 47.71 | C |
| ATOM | 5385 | O | ARG | B | 52 | 35.681 | −101.517 | −14.817 | 1.00 | 48.24 | O |
| ATOM | 5386 | N | CYS | B | 53 | 35.795 | −103.719 | −15.172 | 1.00 | 50.01 | N |
| ATOM | 5387 | CA | CYS | B | 53 | 36.554 | −103.581 | −16.393 | 1.00 | 54.14 | C |
| ATOM | 5388 | CB | CYS | B | 53 | 37.750 | −104.540 | −16.364 | 1.00 | 63.31 | C |
| ATOM | 5389 | SG | CYS | B | 53 | 39.000 | −104.183 | −17.624 | 1.00 | 86.33 | S |
| ATOM | 5390 | C | CYS | B | 53 | 35.658 | −103.850 | −17.595 | 1.00 | 49.46 | C |
| ATOM | 5391 | O | CYS | B | 53 | 35.057 | −104.908 | −17.702 | 1.00 | 43.16 | O |
| ATOM | 5392 | N | TRP | B | 54 | 35.548 | −102.884 | −18.495 | 1.00 | 50.85 | N |
| ATOM | 5393 | CA | TRP | B | 54 | 34.698 | −103.082 | −19.666 | 1.00 | 54.37 | C |
| ATOM | 5394 | CB | TRP | B | 54 | 33.767 | −101.885 | −19.919 | 1.00 | 54.51 | C |
| ATOM | 5395 | CG | TRP | B | 54 | 32.683 | −101.659 | −18.882 | 1.00 | 54.18 | C |
| ATOM | 5396 | CD1 | TRP | B | 54 | 32.276 | −102.316 | −17.885 | 1.00 | 54.37 | C |
| ATOM | 5397 | NE1 | TRP | B | 54 | 31.243 | −101.950 | −17.161 | 1.00 | 53.97 | N |
| ATOM | 5398 | CE2 | TRP | B | 54 | 30.961 | −100.717 | −17.692 | 1.00 | 54.25 | C |
| ATOM | 5399 | CD2 | TRP | B | 54 | 31.844 | −100.503 | −18.779 | 1.00 | 53.42 | C |
| ATOM | 5400 | CE3 | TRP | B | 54 | 31.757 | −99.300 | −19.498 | 1.00 | 51.81 | C |
| ATOM | 5401 | CZ3 | TRP | B | 54 | 30.807 | −98.365 | −19.123 | 1.00 | 49.71 | C |
| ATOM | 5402 | CH2 | TRP | B | 54 | 29.951 | −98.601 | −18.032 | 1.00 | 53.72 | C |
| ATOM | 5403 | CZ2 | TRP | B | 54 | 30.010 | −99.768 | −17.306 | 1.00 | 56.60 | C |
| ATOM | 5404 | C | TRP | B | 54 | 35.566 | −103.392 | −20.870 | 1.00 | 50.79 | C |
| ATOM | 5405 | O | TRP | B | 54 | 36.005 | −102.511 | −21.599 | 1.00 | 52.17 | O |
| ATOM | 5406 | N | ALA | B | 55 | 35.824 | −104.669 | −21.067 | 1.00 | 53.99 | N |
| ATOM | 5407 | CA | ALA | B | 55 | 36.799 | −105.062 | −22.066 | 1.00 | 55.25 | C |
| ATOM | 5408 | CB | ALA | B | 55 | 38.094 | −105.509 | −21.391 | 1.00 | 53.09 | C |
| ATOM | 5409 | C | ALA | B | 55 | 36.251 | −106.152 | −22.969 | 1.00 | 53.39 | C |
| ATOM | 5410 | O | ALA | B | 55 | 35.034 | −106.393 | −23.026 | 1.00 | 48.98 | O |
| ATOM | 5411 | N | GLY | B | SG | 37.171 | −106.801 | −23.672 | 1.00 | 52.41 | N |
| ATOM | 5412 | CA | GLY | B | 56 | 36.836 | −107.825 | −24.636 | 1.00 | 54.87 | C |
| ATOM | 5413 | C | GLY | B | 56 | 38.002 | −108.140 | −24.948 | 1.00 | 51.99 | C |
| ATOM | 5414 | O | GLY | B | 56 | 39.166 | −108.416 | −24.695 | 1.00 | 56.59 | O |
| ATOM | 5415 | N | SER | B | 57 | 37.668 | −109.878 | −25.529 | 1.00 | 48.06 | N |
| ATOM | 5416 | CA | SER | B | 57 | 38.603 | −110.947 | −25.751 | 1.00 | 49.26 | C |
| ATOM | 5417 | CB | SER | B | 57 | 38.667 | −111.843 | −24.516 | 1.00 | 44.78 | C |
| ATOM | 5418 | OG | SER | B | 57 | 37.444 | −112.317 | −24.349 | 1.00 | 48.55 | O |
| ATOM | 5419 | C | SER | B | 57 | 38.235 | −111.768 | −26.984 | 1.00 | 51.52 | C |
| ATOM | 5420 | O | SER | B | 57 | 37.062 | −111.819 | −27.411 | 1.00 | 49.00 | O |
| ATOM | 5421 | N | LEU | B | 58 | 39.258 | −112.400 | −27.555 | 1.00 | 53.96 | N |
| ATOM | 5422 | CA | LEU | B | 58 | 39.080 | −113.262 | −28.715 | 1.00 | 56.94 | C |
| ATOM | 5423 | CB | LEU | B | 58 | 39.752 | −112.639 | −29.950 | 1.00 | 56.06 | C |
| ATOM | 5424 | CG | LEU | B | 58 | 39.375 | −111.199 | −30.365 | 1.00 | 55.08 | C |
| ATOM | 5425 | CD1 | LEU | B | 58 | 40.350 | −110.646 | −31.392 | 1.00 | 54.75 | C |
| ATOM | 5426 | CD2 | LEU | B | 58 | 37.949 | −111.691 | −30.902 | 1.00 | 59.32 | C |
| ATOM | 5427 | C | LEU | B | 58 | 39.597 | −114.680 | −28.399 | 1.00 | 60.19 | C |
| ATOM | 5428 | O | LEU | B | 58 | 40.600 | −114.844 | −27.699 | 1.00 | 60.83 | O |
| ATOM | 5429 | N | TRP | B | 59 | 38.886 | −115.692 | −28.898 | 1.00 | 59.11 | N |
| ATOM | 5430 | CA | TRP | B | 59 | 39.162 | −117.090 | −28.581 | 1.00 | 58.86 | C |
| ATOM | 5431 | CB | TRP | B | 59 | 38.201 | −117.597 | −27.499 | 1.00 | 61.66 | C |
| ATOM | 5432 | CG | TRP | B | 59 | 38.216 | −116.789 | −26.228 | 1.00 | 67.37 | C |
| ATOM | 5433 | CD1 | TRP | B | 59 | 37.444 | −115.703 | −25.942 | 1.00 | 66.43 | C |
| ATOM | 5434 | NE1 | TRP | B | 59 | 37.735 | −115.229 | −24.689 | 1.00 | 66.49 | N |
| ATOM | 5435 | CE2 | TRP | B | 59 | 38.717 | −116.605 | −24.137 | 1.00 | 68.32 | C |
| ATOM | 5436 | CD2 | TRP | B | 59 | 39.047 | −117.602 | −25.080 | 1.00 | 69.61 | C |
| ATOM | 5437 | CE3 | TRP | B | 59 | 40.038 | −117.939 | −24.756 | 1.00 | 68.34 | C |
| ATOM | 5438 | CZ3 | TRP | B | 59 | 40.660 | −117.849 | −23.514 | 1.00 | 67.30 | C |
| ATOM | 5439 | CH2 | TRP | B | 59 | 40.308 | −116.843 | −22.598 | 1.00 | 70.76 | C |
| ATOM | 5440 | CZ2 | TRP | B | 59 | 39.343 | −115.915 | −22.889 | 1.00 | 69.72 | C |
| ATOM | 5441 | C | TRP | B | 59 | 39.054 | −118.003 | −29.808 | 1.00 | 62.33 | C |
| ATOM | 5442 | O | TRP | B | 59 | 38.171 | −117.821 | −30.659 | 1.00 | 59.73 | O |
| ATOM | 5443 | N | LEU | B | 60 | 39.960 | −118.983 | −29.885 | 1.00 | 65.32 | N |
| ATOM | 5444 | CA | LEU | B | 60 | 39.891 | −120.056 | −30.889 | 1.00 | 66.59 | C |
| ATOM | 5445 | CB | LEU | B | 60 | 41.073 | −119.989 | −31.862 | 1.00 | 65.79 | C |
| ATOM | 5446 | CG | LEU | B | 60 | 41.108 | −121.033 | −32.982 | 1.00 | 68.66 | C |
| ATOM | 5447 | CD1 | LEU | B | 60 | 39.847 | −121.047 | −33.842 | 1.00 | 68.50 | C |
| ATOM | 5448 | CD2 | LEU | B | 60 | 42.348 | −120.811 | −33.834 | 1.00 | 72.56 | C |
| ATOM | 5449 | C | LEU | B | 60 | 39.793 | −121.438 | −30.247 | 1.00 | 63.98 | C |
| ATOM | 5450 | O | LEU | B | 60 | 40.612 | −121.799 | −29.393 | 1.00 | 62.36 | O |
| ATOM | 5451 | N | PHE | B | 61 | 38.772 | −122.186 | −30.665 | 1.00 | 65.11 | N |
| ATOM | 5452 | CA | PHE | B | 61 | 38.482 | −123.518 | −30.142 | 1.00 | 66.17 | C |
| ATOM | 5453 | CB | PHE | B | 61 | 37.124 | −123.532 | −29.441 | 1.00 | 61.66 | C |
| ATOM | 5454 | CG | PHE | B | 61 | 37.180 | −123.118 | −28.001 | 1.00 | 64.27 | C |
| ATOM | 5455 | CD1 | PHE | B | 61 | 37.229 | −121.771 | −27.649 | 1.00 | 65.66 | C |
| ATOM | 5456 | CE1 | PHE | B | 61 | 37.280 | −121.388 | −26.321 | 1.00 | 63.55 | C |
| ATOM | 5457 | CZ | PHE | B | 61 | 37.275 | −122.348 | −25.325 | 1.00 | 61.87 | C |
| ATOM | 5458 | CE2 | PHE | B | 61 | 37.220 | −123.692 | −25.660 | 1.00 | 64.80 | C |
| ATOM | 5459 | CD2 | PHE | B | 61 | 37.168 | −124.069 | −26.992 | 1.00 | 64.27 | C |
| ATOM | 5460 | C | PHE | B | 61 | 38.499 | −124.580 | −31.237 | 1.00 | 71.20 | C |
| ATOM | 5461 | O | PHE | B | 61 | 37.505 | −124.745 | −31.965 | 1.00 | 69.73 | O |
| ATOM | 5462 | N | LYS | B | 62 | 39.620 | −125.303 | −31.348 | 1.00 | 75.64 | N |
| ATOM | 5463 | CA | LYS | B | 62 | 39.733 | −126.401 | −32.322 | 1.00 | 73.86 | C |
| ATOM | 5464 | CB | LYS | B | 62 | 41.184 | −126.849 | −32.517 | 1.00 | 73.47 | C |
| ATOM | 5465 | CG | LYS | B | 62 | 41.873 | −126.173 | −33.693 | 1.00 | 71.94 | C |
| ATOM | 5466 | CD | LYS | B | 62 | 43.332 | −125.877 | −33.386 | 1.00 | 73.59 | C |
| ATOM | 5467 | CE | LYS | B | 62 | 44.044 | −125.292 | −34.596 | 1.00 | 76.70 | C |
| ATOM | 5468 | NZ | LYS | B | 62 | 45.383 | −124.742 | −34.246 | 1.00 | 79.53 | N |
| ATOM | 5469 | C | LYS | B | 62 | 38.832 | −127.557 | −31.931 | 1.00 | 70.78 | C |
| ATOM | 5470 | O | LYS | B | 62 | 38.354 | −128.279 | −32.797 | 1.00 | 68.15 | O |
| ATOM | 5471 | N | ASP | B | 63 | 38.605 | −127.710 | −30.625 | 1.00 | 74.32 | N |
| ATOM | 5472 | CA | ASP | B | 63 | 37.529 | −128.557 | −30.098 | 1.00 | 73.63 | C |
| ATOM | 5473 | CB | ASP | B | 63 | 38.004 | −129.373 | −28.878 | 1.00 | 69.99 | C |
| ATOM | 5474 | CG | ASP | B | 63 | 36.875 | −130.187 | −28.224 | 1.00 | 70.47 | C |
| ATOM | 5475 | OD1 | ASP | B | 63 | 35.813 | −130.423 | −28.859 | 1.00 | 71.18 | O |
| ATOM | 5476 | OD2 | ASP | B | 63 | 37.053 | −130.595 | −27.058 | 1.00 | 70.19 | O |
| ATOM | 5477 | C | ASP | B | 63 | 36.324 | −127.670 | −29.736 | 1.00 | 73.80 | C |
| ATOM | 5478 | O | ASP | B | 63 | 36.333 | −126.995 | −28.698 | 1.00 | 70.05 | O |
| ATOM | 5479 | N | PRO | B | 64 | 35.272 | −127.692 | −30.578 | 1.00 | 75.57 | N |
| ATOM | 5480 | CA | PRO | B | 64 | 34.127 | −126.811 | −30.390 | 1.00 | 75.22 | C |
| ATOM | 5481 | CB | PRO | B | 64 | 33.330 | −127.001 | −31.681 | 1.00 | 75.72 | C |
| ATOM | 5482 | CG | PRO | B | 64 | 33.618 | −128.404 | −32.081 | 1.00 | 78.46 | C |
| ATOM | 5483 | CD | PRO | B | 64 | 35.034 | −128.676 | −31.653 | 1.00 | 80.07 | C |
| ATOM | 5484 | C | PRO | B | 64 | 33.274 | −127.192 | −29.177 | 1.00 | 78.38 | C |
| ATOM | 5485 | O | PRO | B | 64 | 32.573 | −126.338 | −28.630 | 1.00 | 78.07 | O |
| ATOM | 5486 | N | CYS | B | 65 | 33.343 | −128.458 | −28.765 | 1.00 | 75.54 | N |
| ATOM | 5487 | CA | CYS | B | 65 | 32.539 | −128.962 | −27.653 | 1.00 | 70.48 | C |
| ATOM | 5488 | CB | CYS | B | 65 | 32.381 | −130.479 | −27.753 | 1.00 | 74.27 | C |
| ATOM | 5489 | SG | CYS | B | 65 | 31.359 | −131.015 | −29.142 | 1.00 | 83.37 | S |
| ATOM | 5490 | C | CYS | B | 65 | 33.125 | −128.585 | −26.302 | 1.00 | 65.15 | C |
| ATOM | 5491 | O | CYS | B | 65 | 32.470 | −128.765 | −25.274 | 1.00 | 62.17 | O |
| ATOM | 5492 | N | ALA | B | 66 | 34.353 | −128.062 | −26.322 | 1.00 | 67.16 | N |
| ATOM | 5493 | CA | ALA | B | 66 | 35.100 | −127.655 | −25.121 | 1.00 | 67.74 | C |
| ATOM | 5494 | CB | ALA | B | 66 | 36.599 | −127.802 | −25.360 | 1.00 | 61.84 | C |
| ATOM | 5495 | C | ALA | B | 66 | 34.782 | −126.213 | −24.722 | 1.00 | 71.06 | C |
| ATOM | 5496 | O | ALA | B | 66 | 34.987 | −125.814 | −23.575 | 1.00 | 72.73 | O |
| ATOM | 5497 | N | ALA | B | 67 | 34.312 | −125.432 | −25.690 | 1.00 | 73.44 | N |
| ATOM | 5498 | CA | ALA | B | 67 | 33.871 | −124.064 | −25.454 | 1.00 | 74.62 | C |
| ATOM | 5499 | CB | ALA | B | 67 | 33.552 | −123.388 | −26.775 | 1.00 | 73.21 | C |
| ATOM | 5500 | C | ALA | B | 67 | 32.655 | −124.056 | −24.518 | 1.00 | 72.90 | C |
| ATOM | 5501 | O | ALA | B | 67 | 31.769 | −124.912 | −24.642 | 1.00 | 74.98 | O |
| ATOM | 5502 | N | PRO | B | 68 | 32.593 | −123.081 | −23.592 | 1.00 | 67.66 | N |
| ATOM | 5503 | CA | PRO | B | 68 | 33.421 | −121.876 | −23.466 | 1.00 | 64.96 | C |
| ATOM | 5504 | CB | PRO | B | 68 | 32.405 | −120.839 | −22.987 | 1.00 | 62.70 | C |
| ATOM | 5505 | CG | PRO | B | 68 | 33.343 | −121.625 | −22.274 | 1.00 | 61.91 | C |
| ATOM | 5506 | CD | PRO | B | 68 | 31.495 | −123.082 | −22.610 | 1.00 | 62.93 | C |
| ATOM | 5507 | C | PRO | B | 68 | 34.591 | −121.930 | −22.476 | 1.00 | 67.13 | C |
| ATOM | 5508 | O | PRO | B | 68 | 35.180 | −120.890 | −22.181 | 1.00 | 66.74 | O |
| ATOM | 5509 | N | ASN | B | 69 | 34.928 | −123.116 | −21.972 | 1.00 | 74.46 | N |
| ATOM | 5510 | CA | ASN | B | 69 | 36.024 | −123.282 | −20.995 | 1.00 | 76.32 | C |
| ATOM | 5511 | CB | ASN | B | 69 | 36.104 | −124.739 | −20.515 | 1.00 | 79.83 | C |
| ATOM | 5512 | CG | ASN | B | 69 | 37.004 | −124.914 | −19.296 | 1.00 | 84.12 | C |
| ATOM | 5513 | OD1 | ASN | B | 69 | 38.120 | −124.377 | −19.225 | 1.00 | 76.10 | O |
| ATOM | 5514 | ND2 | ASN | B | 69 | 36.522 | −125.686 | −18.328 | 1.00 | 84.17 | N |
| ATOM | 5515 | C | ASN | B | 69 | 37.394 | −122.818 | −21.507 | 1.00 | 74.95 | C |
| ATOM | 5516 | O | ASN | B | 69 | 37.905 | −123.147 | −22.493 | 1.00 | 75.84 | O |
| ATOM | 5517 | N | GLU | B | 70 | 37.984 | −121.853 | −20.800 | 1.00 | 75.29 | N |
| ATOM | 5518 | CA | GLU | B | 70 | 39.214 | −121.154 | −21.222 | 1.00 | 72.14 | C |
| ATOM | 5519 | CB | GLU | B | 70 | 39.480 | −119.929 | −20.331 | 1.00 | 70.59 | C |
| ATOM | 5520 | CG | GLU | B | 70 | 38.303 | −118.984 | −20.121 | 1.00 | 74.54 | C |
| ATOM | 5521 | CD | GLU | B | 70 | 37.292 | −119.478 | −19.087 | 1.00 | 80.40 | C |
| ATOM | 5522 | OE1 | GLU | B | 70 | 37.696 | −120.187 | −18.138 | 1.00 | 81.12 | O |
| ATOM | 5523 | OE2 | GLU | B | 70 | 36.085 | −119.156 | −19.220 | 1.00 | 79.46 | O |
| ATOM | 5524 | C | GLU | B | 70 | 40.466 | −122.037 | −21.220 | 1.00 | 74.65 | C |
| ATOM | 5525 | O | GLU | B | 70 | 41.402 | −121.197 | −21.993 | 1.00 | 74.05 | O |
| ATOM | 5526 | N | GLY | B | 71 | 40.479 | −123.036 | −20.336 | 1.00 | 75.79 | N |
| ATOM | 5527 | CA | GLY | B | 71 | 41.588 | −123.991 | −20.212 | 1.00 | 76.90 | C |
| ATOM | 5528 | C | GLY | B | 71 | 41.730 | −124.950 | −21.382 | 1.00 | 76.63 | C |
| ATOM | 5529 | O | GLY | B | 71 | 42.838 | −125.393 | −21.694 | 1.00 | 80.23 | O |
| ATOM | 5530 | N | PHE | B | 72 | 40.609 | −125.264 | −22.030 | 1.00 | 76.43 | N |
| ATOM | 5531 | CA | PHE | B | 72 | 40.604 | −126.157 | −23.190 | 1.00 | 76.57 | C |
| ATOM | 5532 | CB | PHE | B | 72 | 39.519 | −127.244 | −23.066 | 1.00 | 85.06 | C |
| ATOM | 5533 | CG | PHE | B | 72 | 39.420 | −127.876 | −21.698 | 1.00 | 96.46 | C |
| ATOM | 5534 | CD1 | PHE | B | 72 | 40.564 | −128.153 | −20.938 | 1.00 | 97.43 | C |
| ATOM | 5535 | CE1 | PHE | B | 72 | 40.462 | −128.733 | −19.684 | 1.00 | 95.54 | C |
| ATOM | 5536 | CZ | PHE | B | 72 | 39.215 | −129.067 | −19.181 | 1.00 | 98.02 | C |
| ATOM | 5537 | CE2 | PHE | B | 72 | 38.068 | −128.814 | −19.928 | 1.00 | 101.08 | C |
| ATOM | 5538 | CD2 | PHE | B | 72 | 38.173 | −128.226 | −21.180 | 1.00 | 96.64 | C |
| ATOM | 5539 | C | PHE | B | 72 | 40.453 | −125.411 | −24.524 | 1.00 | 71.05 | C |
| ATOM | 5540 | O | PHE | B | 72 | 39.796 | −125.896 | −25.442 | 1.00 | 62.87 | O |
| ATOM | 5541 | N | CYS | B | 73 | 41.063 | −124.238 | −24.638 | 1.00 | 65.91 | N |
| ATOM | 5542 | CA | CYS | B | 73 | 41.050 | −123.536 | −25.911 | 1.00 | 68.70 | C |
| ATOM | 5543 | CB | CYS | B | 73 | 40.790 | −122.039 | −25.714 | 1.00 | 65.55 | C |
| ATOM | 5544 | SG | CYS | B | 73 | 42.212 | −121.052 | −25.170 | 1.00 | 61.04 | S |
| ATOM | 5545 | C | CYS | B | 73 | 42.373 | −123.750 | −26.643 | 1.00 | 69.79 | C |
| ATOM | 5546 | O | CYS | B | 73 | 43.380 | −124.092 | −26.018 | 1.00 | 68.25 | O |
| ATOM | 5547 | N | SER | B | 74 | 42.358 | −123.543 | −27.960 | 1.00 | 66.27 | N |
| ATOM | 5548 | CA | SER | B | 74 | 43.574 | −123.528 | −28.768 | 1.00 | 62.43 | C |
| ATOM | 5549 | CB | SER | B | 74 | 43.242 | −123.795 | −30.238 | 1.00 | 62.44 | C |
| ATOM | 5550 | OG | SER | B | 74 | 42.206 | −124.768 | −30.370 | 1.00 | 62.11 | O |
| ATOM | 5551 | C | SER | B | 74 | 44.320 | −122.194 | −28.606 | 1.00 | 67.33 | C |
| ATOM | 5552 | O | SER | B | 74 | 45.535 | −122.180 | −28.361 | 1.00 | 68.91 | O |
| ATOM | 5553 | N | ALA | B | 75 | 43.597 | −121.075 | −28.739 | 1.00 | 67.04 | N |
| ATOM | 5554 | CA | ALA | B | 75 | 44.181 | −119.734 | −28.518 | 1.00 | 63.80 | C |
| ATOM | 5555 | CB | ALA | B | 75 | 44.750 | −119.167 | −29.816 | 1.00 | 56.92 | C |
| ATOM | 5556 | C | ALA | B | 75 | 43.205 | −118.738 | −27.872 | 1.00 | 61.56 | C |
| ATOM | 5557 | O | ALA | B | 75 | 41.995 | −118.974 | −27.836 | 1.00 | 58.17 | O |
| ATOM | 5558 | N | GLY | B | 76 | 43.756 | −117.630 | −27.372 | 1.00 | 63.02 | N |
| ATOM | 5559 | CA | GLY | B | 76 | 42.999 | −116.590 | −26.657 | 1.00 | 62.34 | C |
| ATOM | 5560 | C | GLY | B | 76 | 43.775 | −115.290 | −26.455 | 1.00 | 62.73 | C |
| ATOM | 5561 | O | GLY | B | 76 | 45.000 | −115.309 | −26.303 | 1.00 | 67.31 | O |
| ATOM | 5562 | N | VAL | B | 77 | 43.070 | −114.157 | −26.474 | 1.00 | 59.01 | N |
| ATOM | 5563 | CA | VAL | B | 77 | 43.707 | −112.855 | −26.239 | 1.00 | 59.09 | C |
| ATOM | 5564 | CB | VAL | B | 77 | 44.520 | −112.380 | −27.477 | 1.00 | 53.25 | C |
| ATOM | 5565 | CG1 | VAL | B | 77 | 43.599 | −111.937 | −28.605 | 1.00 | 53.56 | C |
| ATOM | 5566 | CG2 | VAL | B | 77 | 45.484 | −111.258 | −27.103 | 1.00 | 52.13 | C |
| ATOM | 5567 | C | VAL | B | 77 | 42.741 | −111.749 | −25.729 | 1.00 | 61.91 | C |
| ATOM | 5568 | O | VAL | B | 77 | 41.550 | −111.734 | −26.051 | 1.00 | 62.67 | O |
| ATOM | 5569 | N | GLN | B | 78 | 43.290 | −110.827 | −24.936 | 1.00 | 68.63 | N |
| ATOM | 5570 | CA | GLN | B | 78 | 42.570 | −109.660 | −24.429 | 1.00 | 71.74 | C |
| ATOM | 5571 | CB | GLN | B | 78 | 43.208 | −109.194 | −23.113 | 1.00 | 72.99 | C |
| ATOM | 5572 | CG | GLN | B | 78 | 42.308 | −108.367 | −22.200 | 1.00 | 85.58 | C |
| ATOM | 5573 | CD | GLN | B | 78 | 41.186 | −109.174 | −21.551 | 1.00 | 91.02 | C |
| ATOM | 5574 | OE1 | GLN | B | 78 | 41.285 | −110.394 | −21.389 | 1.00 | 91.83 | O |
| ATOM | 5575 | NE2 | GLN | B | 78 | 40.108 | −108.485 | −21.171 | 1.00 | 85.89 | N |
| ATOM | 5576 | C | GLN | B | 78 | 42.603 | −108.526 | −25.459 | 1.00 | 70.28 | C |
| ATOM | 5577 | O | GLN | B | 78 | 43.644 | −108.261 | −26.057 | 1.00 | 67.58 | O |
| ATOM | 5578 | N | THR | B | 79 | 41.454 | −107.886 | −25.685 | 1.00 | 68.94 | N |
| ATOM | 5579 | CA | THR | B | 79 | 41.377 | −106.656 | −26.476 | 1.00 | 64.97 | C |
| ATOM | 5580 | CB | THR | B | 79 | 40.255 | −106.710 | −27.530 | 1.00 | 68.42 | C |
| ATOM | 5581 | OG1 | THR | B | 79 | 39.009 | −107.054 | −26.907 | 1.00 | 65.59 | O |
| ATOM | 5582 | CG2 | THR | B | 79 | 40.568 | −107.741 | −28.592 | 1.00 | 66.84 | C |
| ATOM | 5583 | C | THR | B | 79 | 41.108 | −105.510 | −25.514 | 1.00 | 63.42 | C |
| ATOM | 5584 | O | THR | B | 79 | 40.465 | −105.720 | −24.479 | 1.00 | 66.48 | O |
| ATOM | 5585 | N | GLU | B | 80 | 41.610 | −104.318 | −25.837 | 1.00 | 57.62 | N |
| ATOM | 5586 | CA | GLU | B | 80 | 41.404 | −103.128 | −24.999 | 1.00 | 59.51 | C |
| ATOM | 5587 | CB | GLU | B | 80 | 41.955 | −101.875 | −25.694 | 1.00 | 57.98 | C |
| ATOM | 5588 | CG | GLU | B | 80 | 41.508 | −101.731 | −27.145 | 1.00 | 62.77 | C |
| ATOM | 5589 | CD | GLU | B | 80 | 41.758 | −100.352 | −27.741 | 1.00 | 67.64 | C |
| ATOM | 5590 | OE1 | GLU | B | 80 | 42.359 | −99.487 | −27.052 | 1.00 | 68.32 | O |
| ATOM | 5591 | OE2 | GLU | B | 80 | 41.348 | −100.143 | −28.915 | 1.00 | 63.81 | O |
| ATOM | 5592 | C | GLU | B | 80 | 39.917 | −102.922 | −24.606 | 1.00 | 59.50 | C |
| ATOM | 5593 | O | GLU | B | 80 | 39.578 | −102.874 | −23.417 | 1.00 | 60.55 | O |
| ATOM | 5594 | N | ALA | B | 81 | 39.043 | −102.837 | −25.609 | 1.00 | 53.52 | N |
| ATOM | 5595 | CA | ALA | B | 81 | 37.620 | −102.582 | −25.393 | 1.00 | 52.59 | C |
| ATOM | 5596 | CB | ALA | B | 81 | 37.197 | −101.317 | −26.122 | 1.00 | 48.50 | C |
| ATOM | 5597 | C | ALA | B | 81 | 36.761 | −103.772 | −25.822 | 1.00 | 49.06 | C |
| ATOM | 5598 | O | ALA | B | 81 | 37.274 | −104.792 | −26.265 | 1.00 | 50.75 | O |
| ATOM | 5599 | N | GLY | B | 82 | 35.450 | −103.635 | −25.663 | 1.00 | 47.91 | N |
| ATOM | 5600 | CA | GLY | B | 82 | 34.538 | −104.733 | −25.914 | 1.00 | 40.65 | C |
| ATOM | 5601 | C | GLY | B | 82 | 34.511 | −105.027 | −27.395 | 1.00 | 43.29 | C |
| ATOM | 5602 | O | GLY | B | 82 | 34.985 | −104.221 | −28.213 | 1.00 | 43.04 | O |
| ATOM | 5603 | N | VAL | B | 83 | 33.959 | −106.187 | −27.743 | 1.00 | 40.31 | N |
| ATOM | 5604 | CA | VAL | B | 83 | 33.813 | −106.549 | −29.129 | 1.00 | 41.79 | C |
| ATOM | 5605 | CB | VAL | B | 83 | 34.458 | −107.935 | −29.456 | 1.00 | 43.54 | C |
| ATOM | 5606 | CG1 | VAL | B | 83 | 35.948 | −107.951 | −29.081 | 1.00 | 42.32 | C |
| ATOM | 5607 | CG2 | VAL | B | 83 | 33.762 | −109.060 | −28.722 | 1.00 | 46.85 | C |
| ATOM | 5608 | C | VAL | B | 83 | 32.326 | −106.476 | −29.451 | 1.00 | 44.91 | C |
| ATOM | 5609 | O | VAL | B | 83 | 31.513 | −107.224 | −28.885 | 1.00 | 43.60 | O |
| ATOM | 5610 | N | ALA | B | 84 | 31.967 | −105.549 | −30.334 | 1.00 | 46.77 | N |
| ATOM | 5611 | CA | ALA | B | 84 | 30.567 | −105.402 | −30.740 | 1.00 | 45.92 | C |
| ATOM | 5612 | CB | ALA | B | 84 | 30.241 | −103.967 | −31.108 | 1.00 | 40.86 | C |
| ATOM | 5613 | C | ALA | B | 84 | 30.224 | −106.337 | −31.886 | 1.00 | 50.06 | C |
| ATOM | 5614 | O | ALA | B | 84 | 29.088 | −106.811 | −31.965 | 1.00 | 48.82 | O |
| ATOM | 5615 | N | ASP | B | 85 | 31.195 | −106.582 | −32.778 | 1.00 | 49.29 | N |
| ATOM | 5616 | CA | ASP | B | 85 | 31.018 | −107.535 | −33.888 | 1.00 | 49.35 | C |
| ATOM | 5617 | CB | ASP | B | 85 | 30.286 | −106.887 | −35.081 | 1.00 | 48.04 | C |
| ATOM | 5618 | CG | ASP | B | 85 | 29.606 | −107.911 | −36.003 | 1.00 | 49.56 | C |
| ATOM | 5619 | OD1 | ASP | B | 85 | 29.375 | −109.078 | −35.580 | 1.00 | 50.15 | O |
| ATOM | 5620 | OD2 | ASP | B | 85 | 29.288 | −107.525 | −37.158 | 1.00 | 51.07 | O |
| ATOM | 5621 | C | ASP | B | 85 | 37.349 | −108.144 | −34.353 | 1.00 | 43.41 | C |
| ATOM | 5622 | O | ASP | B | 85 | 33.414 | −107.582 | −34.126 | 1.00 | 42.50 | O |
| ATOM | 5623 | N | LEU | B | 86 | 32.239 | −109.274 | −35.043 | 1.00 | 50.43 | N |
| ATOM | 5624 | CA | LEU | B | 86 | 33.361 | −110.118 | −35.393 | 1.00 | 53.46 | C |
| ATOM | 5625 | CB | LEU | B | 86 | 33.389 | −111.345 | −34.476 | 1.00 | 52.35 | C |
| ATOM | 5626 | CG | LEU | B | 86 | 34.793 | −111.624 | −33.993 | 1.00 | 51.68 | C |
| ATOM | 5627 | CD1 | LEU | B | 86 | 35.446 | −110.277 | −33.742 | 1.00 | 53.35 | C |
| ATOM | 5628 | CD2 | LEU | B | 86 | 34.783 | −112.475 | −32.739 | 1.00 | 43.81 | C |
| ATOM | 5629 | C | LEU | B | 86 | 33.204 | −110.531 | −36.798 | 1.00 | 55.11 | C |
| ATOM | 5630 | O | LEU | B | 86 | 32.084 | −110.739 | −37.297 | 1.00 | 53.79 | O |
| ATOM | 5631 | N | THR | B | 87 | 34.337 | −110.664 | −37.419 | 1.00 | 57.19 | N |
| ATOM | 5632 | CA | THR | B | 87 | 34.341 | −111.776 | −38.630 | 1.00 | 56.20 | C |
| ATOM | 5633 | CB | THR | B | 87 | 33.781 | −111.924 | −39.848 | 1.00 | 54.78 | C |
| ATOM | 5634 | OG1 | THR | B | 87 | 33.427 | −111.970 | −40.856 | 1.00 | 54.41 | O |
| ATOM | 5635 | CG2 | THR | B | 87 | 34.785 | −110.911 | −40.396 | 1.00 | 53.53 | C |
| ATOM | 5636 | C | THR | B | 87 | 35.707 | −112.336 | −38.979 | 1.00 | 57.27 | C |
| ATOM | 5637 | O | THR | B | 87 | 36.743 | −111.581 | −38.766 | 1.00 | 56.85 | O |
| ATOM | 5638 | N | TRP | B | 88 | 35.683 | −113.554 | −39.518 | 1.00 | 59.12 | N |
| ATOM | 5639 | CA | TRP | B | 88 | 36.860 | −114.185 | −40.114 | 1.00 | 57.50 | C |
| ATOM | 5640 | CB | TRP | B | 88 | 36.537 | −115.609 | −40.564 | 1.00 | 51.93 | C |
| ATOM | 5641 | CG | TRP | B | 88 | 36.580 | −116.647 | −39.503 | 1.00 | 48.77 | C |
| ATOM | 5642 | CD1 | TRP | B | 88 | 35.524 | −117.183 | −39.033 | 1.00 | 47.99 | C |
| ATOM | 5643 | NE1 | TRP | B | 88 | 35.958 | −118.269 | −38.065 | 1.00 | 49.94 | N |
| ATOM | 5644 | CE2 | TRP | B | 88 | 37.315 | −118.121 | −37.905 | 1.00 | 45.78 | C |
| ATOM | 5645 | CD2 | TRP | B | 88 | 37.742 | −117.109 | −38.796 | 1.00 | 46.50 | C |
| ATOM | 5646 | CE3 | TRP | B | 88 | 39.107 | −116.172 | −38.834 | 1.00 | 46.20 | C |
| ATOM | 5647 | CZ3 | TRP | B | 88 | 39.987 | −117.430 | −37.980 | 1.00 | 42.74 | C |
| ATOM | 5648 | CH2 | TRP | B | 88 | 39.535 | −118.423 | −37.094 | 1.00 | 45.00 | C |
| ATOM | 5649 | CZ2 | TRP | B | 88 | 38.205 | −118.189 | −37.046 | 1.00 | 47.93 | C |
| ATOM | 5650 | C | TRP | B | 88 | 37.265 | −113.421 | −41.352 | 1.00 | 57.69 | C |
| ATOM | 5651 | O | TRP | B | 88 | 36.410 | −113.067 | −42.158 | 1.00 | 59.83 | O |
| ATOM | 5652 | N | VAL | B | 89 | 38.561 | −113.155 | −41.486 | 1.00 | 58.41 | N |
| ATOM | 5653 | CA | VAL | B | 89 | 39.156 | −112.157 | −42.781 | 1.00 | 61.05 | C |
| ATOM | 5654 | CB | VAL | B | 89 | 40.093 | −111.146 | −42.730 | 1.00 | 60.94 | C |
| ATOM | 5655 | CG1 | VAL | B | 89 | 40.412 | −111.178 | −44.144 | 1.00 | 60.12 | C |
| ATOM | 5656 | CG2 | VAL | B | 89 | 39.474 | −110.523 | −41.917 | 1.00 | 60.45 | C |
| ATOM | 5657 | C | VAL | B | 89 | 39.976 | −114.082 | −43.185 | 1.00 | 62.90 | C |
| ATOM | 5658 | O | VAL | B | 89 | 40.966 | −114.422 | −42.536 | 1.00 | 65.92 | O |
| ATOM | 5659 | N | GLY | B | 90 | 39.560 | −114.752 | −44.249 | 1.00 | 62.93 | N |
| ATOM | 5660 | CA | GLY | B | 90 | 40.245 | −115.955 | −44.680 | 1.00 | 65.51 | C |
| ATOM | 5661 | C | GLY | B | 90 | 40.162 | −117.030 | −43.618 | 1.00 | 70.19 | C |
| ATOM | 5662 | O | GLY | B | 90 | 39.073 | −117.117 | −43.094 | 1.00 | 76.58 | O |
| ATOM | 5663 | N | AGLU | B | 91 | 41.312 | −117.608 | −43.284 | 0.50 | 67.86 | N |
| ATOM | 5664 | N | BGLU | B | 91 | 41.308 | −117.628 | −43.302 | 0.50 | 66.02 | N |
| ATOM | 5665 | CA | AGLU | B | 91 | 41.374 | −118.712 | −42.331 | 0.50 | 68.13 | C |
| ATOM | 5666 | CA | BGLU | B | 91 | 41.384 | −118.730 | −42.343 | 0.50 | 65.12 | C |
| ATOM | 5667 | CB | AGLU | B | 91 | 41.990 | 119.955 | −42.991 | 0.50 | 70.16 | C |
| ATOM | 5668 | CB | BGLU | B | 91 | 41.998 | −119.981 | −43.005 | 0.50 | 64.51 | C |
| ATOM | 5669 | CG | AGLU | B | 91 | 41.696 | −120.088 | −44.478 | 0.50 | 67.57 | C |
| ATOM | 5670 | CG | BGLU | B | 91 | 41.039 | −121.138 | −43.289 | 0.50 | 59.02 | C |
| ATOM | 5671 | CD | AGLU | B | 91 | 42.375 | −119.605 | −45.298 | 0.50 | 65.13 | C |
| ATOM | 5672 | CD | BGLU | B | 91 | 39.804 | −120.728 | −44.066 | 0.50 | 56.64 | C |
| ATOM | 5673 | OE1 | AGLU | B | 91 | 43.499 | −118.605 | −44.939 | 0.50 | 62.09 | O |
| ATOM | 5674 | OE1 | BGLU | B | 91 | 39.795 | −120.898 | −45.299 | 0.50 | 54.80 | O |
| ATOM | 5675 | OE2 | AGLU | B | 91 | 41.787 | −118.550 | −46.301 | 0.50 | 64.70 | O |
| ATOM | 5676 | OE2 | BGLU | B | 91 | 38.839 | −120.140 | −43.446 | 0.50 | 56.26 | O |
| ATOM | 5677 | C | AGLU | B | 91 | 42.170 | −118.340 | −41.082 | 0.50 | 67.29 | C |
| ATOM | 5678 | C | BGLU | B | 91 | 42.183 | −118.351 | −41.092 | 0.50 | 65.46 | C |
| ATOM | 5679 | O | AGLU | B | 91 | 42.133 | −119.057 | −40.079 | 0.50 | 64.27 | O |
| ATOM | 5680 | O | BGLU | B | 91 | 42.155 | −119.074 | −40.092 | 0.50 | 62.61 | O |
| ATOM | 5681 | N | ARG | B | 92 | 42.893 | −117.125 | −41.155 | 1.00 | 66.84 | N |
| ATOM | 5682 | CA | ARG | B | 92 | 43.744 | −116.769 | −40.049 | 1.00 | 67.02 | C |
| ATOM | 5683 | CB | ARG | B | 92 | 45.215 | −116.870 | −40.452 | 1.00 | 73.16 | C |
| ATOM | 5684 | CG | ARG | B | 92 | 45.816 | −118.258 | −40.349 | 1.00 | 80.20 | C |
| ATOM | 5685 | CD | ARG | B | 92 | 47.334 | −118.151 | −40.278 | 1.00 | 87.72 | C |
| ATOM | 5686 | NE | ARG | B | 92 | 47.975 | −119.464 | −40.240 | 1.00 | 93.30 | N |
| ATOM | 5687 | CZ | ARG | B | 92 | 48.346 | −120.156 | −41.316 | 1.00 | 92.04 | C |
| ATOM | 5688 | NH1 | ARG | B | 92 | 48.139 | −119.667 | −42.538 | 1.00 | 90.50 | N |
| ATOM | 5689 | NH2 | ARG | B | 92 | 48.924 | −121.341 | −41.169 | 1.00 | 86.09 | N |
| ATOM | 5690 | C | ARG | B | 92 | 43.452 | −115.332 | −39.561 | 1.00 | 67.73 | C |
| ATOM | 5691 | O | ARG | B | 92 | 43.964 | −114.500 | −38.514 | 1.00 | 62.28 | O |
| ATOM | 5692 | N | GLY | B | 93 | 42.654 | −114.590 | −40.330 | 1.00 | 64.44 | N |
| ATOM | 5693 | CA | GLY | B | 93 | 42.340 | −113.202 | −40.002 | 1.00 | 41.13 | C |
| ATOM | 5694 | C | GLY | B | 93 | 41.065 | −113.072 | −39.180 | 1.00 | 59.46 | C |
| ATOM | 5695 | O | GLY | B | 93 | 40.082 | −113.787 | −39.417 | 1.00 | 54.10 | O |
| ATOM | 5696 | N | ILE | B | 94 | 41.096 | −112.166 | −38.203 | 1.00 | 57.53 | N |
| ATOM | 5697 | CA | ILE | B | 94 | 39.914 | −111.836 | −37.389 | 1.00 | 54.08 | C |
| ATOM | 5698 | CB | ILE | B | 94 | 40.043 | −112.345 | −35.945 | 1.00 | 54.86 | C |
| ATOM | 5699 | CG1 | ILE | B | 94 | 40.244 | −113.864 | −35.944 | 1.00 | 55.49 | C |
| ATOM | 5700 | CD1 | ILE | B | 94 | 41.274 | −114.323 | −34.939 | 1.00 | 51.82 | C |
| ATOM | 5701 | CG2 | ILE | B | 94 | 38.802 | −111.986 | −35.142 | 1.00 | 55.06 | C |
| ATOM | 5702 | C | ILE | B | 94 | 39.689 | −110.334 | −37.378 | 1.00 | 50.67 | C |
| ATOM | 5703 | O | ILE | B | 94 | 40.533 | −109.565 | −36.886 | 1.00 | 43.90 | O |
| ATOM | 5704 | N | LEU | B | 95 | 38.551 | −109.924 | −37.937 | 1.00 | 50.80 | N |
| ATOM | 5705 | CA | LEU | B | 95 | 38.243 | −108.503 | −38.068 | 1.00 | 51.25 | C |
| ATOM | 5706 | CB | LEU | B | 95 | 37.784 | −108.180 | −39.488 | 1.00 | 44.58 | C |
| ATOM | 5707 | CG | LEU | B | 95 | 37.586 | −106.699 | −39.758 | 1.00 | 42.82 | C |
| ATOM | 5708 | CD1 | LEU | B | 95 | 38.915 | −105.947 | −39.730 | 1.00 | 42.64 | C |
| ATOM | 5709 | CD2 | LEU | B | 95 | 36.869 | 106.516 | −41.082 | 1.00 | 39.58 | C |
| ATOM | 5710 | C | LEU | B | 95 | 37.215 | −108.075 | −37.004 | 1.00 | 52.47 | C |
| ATOM | 5711 | O | LEU | B | 95 | 36.124 | −108.668 | −36.883 | 1.00 | 51.94 | O |
| ATOM | 5712 | N | VAL | B | 96 | 37.586 | −107.056 | −36.234 | 1.00 | 48.50 | N |
| ATOM | 5713 | CA | VAL | B | 96 | 36.867 | −106.727 | −34.999 | 1.00 | 50.03 | C |
| ATOM | 5714 | CB | VAL | B | 96 | 37.786 | −106.901 | −33.769 | 1.00 | 49.73 | C |
| ATOM | 5715 | CG1 | VAL | B | 96 | 37.091 | −106.470 | −32.490 | 1.00 | 45.99 | C |
| ATOM | 5716 | CG2 | VAL | B | 96 | 38.236 | −108.343 | −33.661 | 1.00 | 53.02 | C |
| ATOM | 5717 | C | VAL | B | 96 | 36.257 | −105.322 | −35.000 | 1.00 | 47.30 | C |
| ATOM | 5718 | O | VAL | B | 96 | 36.947 | −104.329 | −35.320 | 1.00 | 44.51 | O |
| ATOM | 5719 | N | ALA | B | 97 | 34.963 | −105.263 | −34.660 | 1.00 | 42.46 | N |
| ATOM | 5720 | CA | ALA | B | 97 | 34.253 | −103.995 | −34.416 | 1.00 | 41.57 | C |
| ATOM | 5721 | CB | ALA | B | 97 | 32.788 | −104.097 | −34.846 | 1.00 | 42.70 | C |
| ATOM | 5722 | C | ALA | B | 97 | 34.352 | −103.652 | −32.927 | 1.00 | 41.26 | C |
| ATOM | 5723 | O | ALA | B | 97 | 33.706 | −104.305 | −32.078 | 1.00 | 41.62 | O |
| ATOM | 5724 | N | SER | B | 98 | 35.157 | −102.045 | −32.598 | 1.00 | 38.90 | N |
| ATOM | 5725 | CA | SER | B | 98 | 35.332 | −102.311 | −31.202 | 1.00 | 42.17 | C |
| ATOM | 5726 | CB | SER | B | 98 | 36.801 | −102.119 | −30.901 | 1.00 | 40.48 | C |
| ATOM | 5727 | OG | SER | B | 98 | 36.969 | −101.528 | −29.630 | 1.00 | 42.09 | O |
| ATOM | 5728 | C | SER | B | 98 | 34.535 | −101.096 | −30.685 | 1.00 | 43.84 | C |
| ATOM | 5729 | O | SER | B | 98 | 34.391 | −100.086 | −31.390 | 1.00 | 43.91 | O |
| ATOM | 5730 | N | ASP | B | 99 | 34.079 | −101.208 | −29.432 | 1.00 | 41.97 | N |
| ATOM | 5731 | CA | ASP | B | 99 | 33.619 | −100.069 | −28.619 | 1.00 | 45.30 | C |
| ATOM | 5732 | CB | ASP | B | 99 | 33.533 | −100.483 | −27.153 | 1.00 | 41.29 | C |
| ATOM | 5733 | CG | ASP | B | 99 | 32.333 | −101.329 | −26.860 | 1.00 | 40.81 | C |
| ATOM | 5734 | OD1 | ASP | B | 99 | 31.361 | −101.222 | −27.619 | 1.00 | 46.09 | O |
| ATOM | 5735 | OD2 | ASP | B | 99 | 32.333 | −102.081 | −25.858 | 1.00 | 39.83 | O |
| ATOM | 5736 | C | ASP | B | 99 | 34.507 | −98.816 | −28.722 | 1.00 | 49.99 | C |
| ATOM | 5737 | O | ASP | B | 99 | 34.004 | −97.690 | −28.683 | 1.00 | 54.23 | O |
| ATOM | 5738 | N | SER | B | 100 | 35.820 | −99.022 | −28.856 | 1.00 | 49.39 | N |
| ATOM | 5739 | CA | SER | B | 100 | 36.813 | −97.935 | −28.826 | 1.00 | 49.13 | C |
| ATOM | 5740 | CB | SER | B | 100 | 38.237 | −98.516 | −28.766 | 1.00 | 52.42 | C |
| ATOM | 5741 | OG | SER | B | 100 | 38.542 | −99.333 | −29.903 | 1.00 | 55.48 | O |
| ATOM | 5742 | C | SRR | B | 100 | 36.705 | −96.935 | −29.981 | 1.00 | 49.01 | C |
| ATOM | 5743 | O | SER | B | 100 | 37.349 | −95.876 | −29.952 | 1.00 | 48.38 | O |
| ATOM | 5744 | N | GLY | B | 101 | 35.886 | −97.263 | −30.981 | 1.00 | 50.84 | N |
| ATOM | 5745 | CA | GLY | B | 101 | 35.845 | −96.509 | −32.244 | 1.00 | 51.68 | C |
| ATOM | 5746 | C | GLY | B | 101 | 36.693 | −97.183 | −33.317 | 1.00 | 50.34 | C |
| ATOM | 5747 | O | GLY | B | 101 | 36.683 | −96.802 | −34.507 | 1.00 | 45.27 | O |
| ATOM | 5748 | N | ALA | B | 102 | 37.418 | −98.209 | −32.891 | 1.00 | 51.11 | N |
| ATOM | 5749 | CA | ALA | B | 102 | 38.379 | −98.845 | −33.763 | 1.00 | 56.19 | C |
| ATOM | 5750 | CB | ALA | B | 102 | 39.635 | −99.242 | −32.996 | 1.00 | 54.35 | C |
| ATOM | 5751 | C | ALA | B | 102 | 37.798 | −100.038 | −34.495 | 1.00 | 55.69 | C |
| ATOM | 5752 | O | ALA | B | 102 | 36.939 | −100.781 | −33.975 | 1.00 | 55.57 | O |
| ATOM | 5753 | N | VAL | B | 103 | 38.267 | −100.185 | −35.728 | 1.00 | 54.81 | N |
| ATOM | 5754 | CA | VAL | B | 103 | 38.209 | −101.449 | −36.424 | 1.00 | 52.81 | C |
| ATOM | 5755 | CB | VAL | B | 103 | 37.888 | −101.256 | −37.906 | 1.00 | 52.16 | C |
| ATOM | 5756 | CG1 | VAL | B | 103 | 37.906 | −102.590 | −38.632 | 1.00 | 55.23 | C |
| ATOM | 5757 | CG2 | VAL | B | 103 | 36.521 | −100.624 | −38.049 | 1.00 | 50.21 | C |
| ATOM | 5758 | C | VAL | B | 103 | 39.589 | −102.344 | −36.247 | 1.00 | 55.29 | C |
| ATOM | 5759 | O | VAL | B | 103 | 40.597 | −101.381 | −36.508 | 1.00 | 53.32 | O |
| ATOM | 5760 | N | GLU | B | 104 | 39.653 | −103.272 | −35.748 | 1.00 | 60.95 | N |
| ATOM | 5761 | CA | GLU | B | 104 | 40.958 | −103.932 | −35.677 | 1.00 | 60.99 | C |
| ATOM | 5762 | CB | GLU | B | 104 | 41.505 | −103.997 | −34.248 | 1.00 | 63.20 | C |
| ATOM | 5763 | CG | GLU | B | 104 | 40.571 | −104.575 | −33.203 | 1.00 | 67.97 | C |
| ATOM | 5764 | CD | GLU | B | 104 | 40.969 | −104.165 | −31.793 | 1.00 | 71.48 | C |
| ATOM | 5765 | OE1 | GLU | B | 104 | 41.869 | −103.311 | −31.642 | 1.00 | 77.45 | O |
| ATOM | 5766 | OE2 | GLU | B | 104 | 40.379 | −104.589 | −30.831 | 1.00 | 74.06 | O |
| ATOM | 5767 | C | GLU | B | 104 | 41.033 | −105.275 | −36.384 | 1.00 | 56.46 | C |
| ATOM | 5768 | O | GLU | B | 104 | 40.064 | −106.345 | −36.417 | 1.00 | 58.51 | O |
| ATOM | 5769 | N | LEU | B | 105 | 42.193 | −105.503 | −36.990 | 1.00 | 59.13 | N |
| ATOM | 5770 | CA | LEU | B | 105 | 42.549 | −106.785 | −37.387 | 1.00 | 58.31 | C |
| ATOM | 5771 | CB | LEU | B | 105 | 43.176 | −106.597 | −38.966 | 1.00 | 55.08 | C |
| ATOM | 5772 | CG | LEU | B | 105 | 43.569 | −107.352 | −39.760 | 1.00 | 54.00 | C |
| ATOM | 5773 | CD1 | LEU | B | 105 | 47.385 | −108.782 | −40.024 | 1.00 | 46.73 | C |
| ATOM | 5774 | CD2 | LEU | B | 105 | 44.251 | −107.454 | −41.065 | 1.00 | 49.72 | C |
| ATOM | 5775 | C | LEU | B | 105 | 43.531 | −107.501 | −36.687 | 1.00 | 57.78 | C |
| ATOM | 5776 | O | LEU | B | 105 | 44.607 | −106.572 | −36.364 | 1.00 | 55.27 | O |
| ATOM | 5777 | N | TRP | B | 106 | 43.120 | −108.592 | −36.269 | 1.00 | 58.28 | N |
| ATOM | 5778 | CA | TRP | B | 106 | 43.955 | −109.508 | −35.512 | 1.00 | 64.66 | C |
| ATOM | 5779 | CB | TRP | B | 106 | 43.216 | −110.112 | −34.261 | 1.00 | 63.89 | C |
| ATOM | 5780 | CG | TRP | B | 106 | 43.037 | −109.951 | −33.209 | 1.00 | 65.51 | C |
| ATOM | 5781 | CD1 | TRP | B | 106 | 42.125 | −108.946 | −33.224 | 1.00 | 65.38 | C |
| ATOM | 5782 | NE1 | TRP | B | 106 | 42.268 | −107.261 | −32.112 | 1.00 | 68.29 | N |
| ATOM | 5783 | CE2 | TRP | B | 106 | 43.283 | −107.751 | −31.338 | 1.00 | 65.34 | C |
| ATOM | 5784 | CD2 | TRP | B | 106 | 43.800 | −108.583 | −32.000 | 1.00 | 66.87 | C |
| ATOM | 5785 | CE3 | TRP | B | 106 | 44.867 | −109.574 | −31.416 | 1.00 | 66.51 | C |
| ATOM | 5786 | CZ3 | TRP | B | 106 | 45.376 | −109.113 | −30.199 | 1.00 | 68.73 | C |
| ATOM | 5787 | CH2 | TRP | B | 106 | 44.835 | −107.979 | −29.568 | 1.00 | 66.02 | C |
| ATOM | 5788 | CZ2 | TRP | B | 106 | 43.796 | −107.286 | −30.123 | 1.00 | 64.52 | C |
| ATOM | 5789 | C | TRP | B | 106 | 44.358 | −110.775 | −36.422 | 1.00 | 69.77 | C |
| ATOM | 5790 | O | TRP | B | 106 | 43.514 | −111.147 | −37.153 | 1.00 | 68.02 | O |
| ATOM | 5791 | N | GLU | B | 107 | 45.650 | −111.101 | −36.389 | 1.00 | 69.34 | N |
| ATOM | 5792 | CA | GLU | B | 107 | 46.215 | −112.200 | −37.187 | 1.00 | 75.38 | C |
| ATOM | 5793 | CB | GLU | B | 107 | 47.551 | −111.175 | −37.831 | 1.00 | 78.57 | C |
| ATOM | 5794 | CG | GLU | B | 107 | 47.929 | −112.109 | −39.118 | 1.00 | 88.81 | C |
| ATOM | 5795 | CD | GLU | B | 107 | 47.318 | −111.906 | −40.382 | 1.00 | 92.66 | C |
| ATOM | 5796 | OE1 | GLU | B | 107 | 47.505 | −110.693 | −40.636 | 1.00 | 95.47 | O |
| ATOM | 5797 | OE2 | GLU | B | 307 | 46.665 | −112.655 | −41.144 | 1.00 | 92.03 | O |
| ATOM | 5798 | C | GLU | B | 107 | 46.402 | −113.443 | −36.310 | 1.00 | 71.05 | C |
| ATOM | 5799 | O | GLU | B | 107 | 46.779 | −113.152 | −35.135 | 1.00 | 71.02 | O |
| ATOM | 5800 | N | LEU | B | 108 | 46.118 | −114.606 | −36.873 | 1.00 | 74.21 | N |
| ATOM | 5801 | CA | LEU | B | 108 | 46.342 | −115.853 | −36.161 | 1.00 | 75.03 | C |
| ATOM | 5802 | CB | LEU | B | 108 | 45.405 | −116.929 | −36.700 | 1.00 | 69.06 | C |
| ATOM | 5803 | CG | LEU | B | 108 | 44.970 | −118.697 | −35.826 | 1.00 | 67.07 | C |
| ATOM | 5804 | CD1 | LEU | B | 108 | 44.761 | −117.664 | −34.389 | 1.00 | 68.92 | C |
| ATOM | 5805 | CD2 | LEU | B | 108 | 43.690 | −118.687 | −36.395 | 1.00 | 66.46 | C |
| ATOM | 5806 | C | LEU | B | 108 | 47.816 | −116.792 | −36.246 | 1.00 | 77.98 | C |
| ATOM | 5807 | O | LEU | B | 108 | 48.529 | −115.954 | −37.195 | 1.00 | 78.20 | O |
| ATOM | 5808 | N | ASP | B | 109 | 48.255 | −117.624 | −35.225 | 1.00 | 81.34 | N |
| ATOM | 5809 | CA | ASP | B | 109 | 49.602 | −117.601 | −35.137 | 1.00 | 83.51 | C |
| ATOM | 5810 | CB | ASP | B | 109 | 49.676 | −118.425 | −33.849 | 1.00 | 77.06 | C |
| ATOM | 5811 | CG | ASP | B | 109 | 51.025 | −118.167 | −33.177 | 1.00 | 75.75 | C |
| ATOM | 5812 | OD1 | ASP | B | 109 | 51.907 | −117.604 | −33.629 | 1.00 | 71.99 | O |
| ATOM | 5813 | OD2 | ASP | B | 109 | 51.189 | −119.688 | −32.168 | 1.00 | 76.57 | O |
| ATOM | 5814 | C | ASP | B | 109 | 49.947 | −118.527 | −36.309 | 1.00 | 87.08 | C |
| ATOM | 5815 | O | ASP | B | 109 | 49.056 | −119.066 | −36.982 | 1.00 | 88.10 | O |
| ATOM | 5816 | N | GLU | B | 110 | 51.243 | −118.727 | −36.537 | 1.00 | 93.37 | N |
| ATOM | 5817 | CA | GLU | B | 110 | 51.708 | −119.781 | −37.445 | 1.00 | 93.19 | C |
| ATOM | 5818 | C3 | GLU | B | 110 | 53.223 | −119.672 | −37.673 | 1.00 | 55.61 | C |
| ATOM | 5819 | CG | GLU | B | 110 | 53.729 | −120.409 | −38.908 | 1.00 | 104.01 | C |
| ATOM | 5820 | CD | GLU | B | 110 | 53.310 | −119.746 | −40.214 | 1.00 | 108.41 | C |
| ATOM | 5821 | OE1 | GLU | B | 110 | 53.844 | −118.662 | −40.539 | 1.00 | 105.68 | O |
| ATOM | 5822 | OE2 | GLU | B | 110 | 52.453 | −120.316 | −40.924 | 1.00 | 109.59 | O |
| ATOM | 5823 | C | GLU | B | 110 | 51.307 | −121.173 | −36.904 | 1.00 | 85.85 | C |
| ATOM | 5824 | O | GLU | B | 110 | 50.695 | −121.976 | −37.620 | 1.00 | 74.75 | O |
| ATOM | 5825 | N | ASN | B | 111 | 51.627 | −121.431 | −35.632 | 1.00 | 80.97 | N |
| ATOM | 5826 | CA | ASN | B | 111 | 51.203 | −122.650 | −34.937 | 1.00 | 80.19 | C |
| ATOM | 5827 | CB | ASN | B | 111 | 51.920 | −122.789 | −33.596 | 1.00 | 84.20 | C |
| ATOM | 5828 | CG | ASN | B | 111 | 53.346 | −122.285 | −33.637 | 1.00 | 88.96 | C |
| ATOM | 5829 | OD1 | ASN | B | 111 | 53.603 | −121.116 | −33.950 | 1.00 | 91.63 | O |
| ATOM | 5830 | ND2 | ASN | B | 111 | 54.285 | −123.160 | −33.303 | 1.00 | 86.31 | N |
| ATOM | 5831 | C | ASN | B | 111 | 49.713 | −122.633 | −34.659 | 1.00 | 77.46 | C |
| ATOM | 5832 | O | ASN | B | 111 | 49.115 | −123.683 | −34.392 | 1.00 | 74.75 | O |
| ATOM | 5833 | N | GLU | B | 112 | 49.144 | −121.423 | −34.715 | 1.00 | 77.89 | N |
| ATOM | 5834 | CA | GLU | B | 112 | 47.735 | −121.122 | −34.401 | 1.00 | 70.23 | C |
| ATOM | 5835 | CB | GLU | B | 112 | 46.772 | −121.931 | −35.284 | 1.00 | 67.30 | C |
| ATOM | 5836 | CG | GLU | B | 112 | 46.947 | −121.664 | −36.770 | 1.00 | 64.55 | C |
| ATOM | 5837 | CD | GLU | B | 112 | 45.829 | −122.252 | −37.617 | 1.00 | 64.45 | C |
| ATOM | 5838 | OE1 | GLU | B | 112 | 45.052 | −123.093 | −37.107 | 1.00 | 57.22 | O |
| ATOM | 5839 | OE2 | GLU | B | 112 | 45.738 | −121.868 | −38.805 | 1.00 | 65.46 | O |
| ATOM | 5840 | C | GLU | B | 112 | 47.450 | −121.307 | −32.906 | 1.00 | 68.36 | C |
| ATOM | 5841 | O | GLU | B | 112 | 46.430 | −121.898 | −32.515 | 1.00 | 68.28 | O |
| ATOM | 5842 | N | THR | B | 113 | 48.369 | −120.791 | −32.088 | 1.00 | 63.09 | N |
| ATOM | 5843 | CA | THR | B | 113 | 48.350 | −120.976 | −30.631 | 1.00 | 71.03 | C |
| ATOM | 5844 | CB | THR | B | 113 | 49.520 | −121.865 | −30.121 | 1.00 | 70.56 | C |
| ATOM | 5845 | OG1 | THR | B | 113 | 50.763 | −121.384 | −30.655 | 1.00 | 65.84 | O |
| ATOM | 5846 | CG2 | THR | B | 113 | 49.310 | −123.341 | −30.501 | 1.00 | 70.31 | C |
| ATOM | 5847 | C | THR | B | 113 | 48.427 | −119.637 | −29.912 | 1.00 | 70.98 | C |
| ATOM | 5848 | O | THR | B | 113 | 48.414 | −119.583 | −78.679 | 1.00 | 69.74 | O |
| ATOM | 5849 | N | LEU | B | 114 | 48.552 | −118.566 | −30.693 | 1.00 | 73.88 | N |
| ATOM | 5850 | CA | LEU | B | 114 | 48.375 | 117.207 | −30.183 | 1.00 | 70.79 | C |
| ATOM | 5851 | CB | LEU | B | 114 | 49.660 | −116.650 | −29.536 | 1.00 | 82.57 | C |
| ATOM | 5852 | CG | LEU | B | 114 | 50.965 | −116.317 | −30.260 | 1.00 | 83.70 | C |
| ATOM | 5853 | CD1 | LEU | B | 114 | 50.823 | −115.041 | −31.079 | 1.00 | 91.55 | C |
| ATOM | 5854 | CD2 | LEU | B | 114 | 52.084 | −116.157 | −29.240 | 1.00 | 83.00 | C |
| ATOM | 5855 | C | LEU | B | 114 | 47.757 | −116.268 | −31.233 | 1.00 | 73.23 | C |
| ATOM | 5856 | O | LEU | B | 114 | 47.735 | −116.576 | −32.437 | 1.00 | 73.81 | O |
| ATOM | 5857 | N | ILE | B | 115 | 47.218 | −115.145 | −30.755 | 1.00 | 63.30 | N |
| ATOM | 5858 | CA | ILE | B | 115 | 46.455 | −114.212 | −31.597 | 1.00 | 53.15 | C |
| ATOM | 5859 | CB | ILE | B | 115 | 44.940 | −114.192 | −31.233 | 1.00 | 53.75 | C |
| ATOM | 5860 | CG1 | ILE | B | 115 | 44.347 | −115.606 | −31.279 | 1.00 | 53.39 | C |
| ATOM | 5861 | CD1 | ILE | B | 115 | 42.905 | −115.729 | −30.821 | 1.00 | 53.73 | C |
| ATOM | 5862 | CG2 | ILE | B | 115 | 44.172 | −113.274 | −32.174 | 1.00 | 53.26 | C |
| ATOM | 5863 | C | ILE | B | 115 | 47.082 | −112.828 | −31.482 | 1.00 | 53.11 | C |
| ATOM | 5864 | O | ILE | B | 115 | 47.268 | −112.303 | −30.379 | 1.00 | 43.24 | O |
| ATOM | 5865 | N | VAL | B | 116 | 47.413 | −112.237 | −32.620 | 1.00 | 41.29 | N |
| ATOM | 5866 | CA | VAL | B | 116 | 48.282 | −111.070 | −32.593 | 1.00 | 51.68 | C |
| ATOM | 5867 | CB | VAL | B | 116 | 49.760 | −111.461 | −32.990 | 1.00 | 55.64 | C |
| ATOM | 5868 | CG1 | VAL | B | 116 | 49.834 | −112.118 | −34.369 | 1.00 | 55.29 | C |
| ATOM | 5869 | CG2 | VAL | B | 116 | 50.739 | −110.285 | −32.865 | 1.00 | 53.74 | C |
| ATOM | 5870 | C | VAL | B | 116 | 47.677 | −109.905 | −33.374 | 1.00 | 52.15 | C |
| ATOM | 5871 | O | VAL | B | 116 | 47.213 | −110.060 | −34.511 | 1.00 | 52.20 | O |
| ATOM | 5872 | N | SER | B | 117 | 47.642 | −108.742 | −32.732 | 1.00 | 53.01 | N |
| ATOM | 5873 | CA | SER | B | 117 | 47.095 | −107.552 | −33.367 | 1.00 | 56.54 | C |
| ATOM | 5874 | CE | SER | B | 117 | 47.046 | −106.361 | −32.397 | 1.00 | 56.29 | C |
| ATOM | 5875 | OG | SER | B | 117 | 46.914 | −105.124 | −33.093 | 1.00 | 50.72 | O |
| ATOM | 5876 | C | SER | B | 117 | 47.946 | −107.217 | −34.573 | 1.00 | 62.07 | C |
| ATOM | 5877 | O | SER | B | 117 | 49.151 | −107.016 | −34.437 | 1.00 | 59.85 | O |
| ATOM | 5878 | N | LYS | B | 118 | 47.305 | −107.184 | −35.742 | 1.00 | 67.80 | N |
| ATOM | 5879 | CA | LYS | B | 118 | 47.933 | −106.723 | −36.977 | 1.00 | 72.01 | C |
| ATOM | 5880 | CE | LYS | B | 118 | 47.491 | −107.587 | −38.167 | 1.00 | 76.76 | C |
| ATOM | 5881 | CG | LYS | B | 118 | 48.171 | −107.254 | −39.497 | 1.00 | 91.87 | C |
| ATOM | 5882 | CD | LYS | B | 118 | 49.680 | −107.517 | −39.503 | 1.00 | 98.52 | C |
| ATOM | 5883 | CE | LYS | B | 118 | 50.353 | −106.915 | −40.736 | 1.00 | 100.53 | C |
| ATOM | 5884 | NZ | LYS | B | 118 | 51.827 | −107.149 | −40.790 | 1.00 | 97.24 | N |
| ATOM | 5885 | C | LYS | B | 118 | 47.698 | −105.218 | −37.239 | 1.00 | 71.92 | C |
| ATOM | 5886 | O | LYS | B | 118 | 48.640 | −104.496 | −37.592 | 1.00 | 70.46 | O |
| ATOM | 5887 | N | PHE | B | 119 | 46.454 | −104.750 | −37.092 | 1.00 | 69.86 | N |
| ATOM | 5888 | CA | PHE | B | 119 | 46.179 | −103.296 | −37.056 | 1.00 | 63.76 | C |
| ATOM | 5889 | CB | PHE | B | 119 | 46.089 | −102.649 | −38.461 | 1.00 | 61.26 | C |
| ATOM | 5890 | CG | PHE | B | 119 | 44.788 | −102.900 | −39.183 | 1.00 | 65.11 | C |
| ATOM | 5891 | CD1 | PHE | B | 119 | 43.623 | −102.208 | −38.830 | 1.00 | 60.22 | C |
| ATOM | 5892 | CE1 | PHE | B | 119 | 42.431 | −102.448 | −39.490 | 1.00 | 58.00 | C |
| ATOM | 5893 | CZ | PHE | B | 119 | 42.386 | −103.157 | −40.535 | 1.00 | 60.05 | C |
| ATOM | 5894 | CE2 | PHE | B | 119 | 43.535 | −104.031 | −40.914 | 1.00 | 60.93 | C |
| ATOM | 5895 | CD2 | PHE | B | 119 | 44.730 | −103.103 | −40.242 | 1.00 | 60.67 | C |
| ATOM | 5896 | C | PHE | B | 119 | 44.984 | −102.903 | −36.181 | 1.00 | 59.48 | C |
| ATOM | 5897 | O | PHE | B | 119 | 43.999 | −103.650 | −36.052 | 1.00 | 47.71 | O |
| ATOM | 5898 | N | CYS | B | 120 | 45.106 | −101.719 | −35.588 | 1.00 | 54.76 | N |
| ATOM | 5899 | CA | CYS | B | 120 | 44.064 | −101.132 | −34.762 | 1.00 | 55.05 | C |
| ATOM | 5900 | CB | CYS | B | 120 | 44.473 | −101.170 | −33.282 | 1.00 | 56.57 | C |
| ATOM | 5901 | SG | CYS | B | 120 | 43.218 | −100.644 | −32.075 | 1.00 | 56.57 | S |
| ATOM | 5902 | C | CYS | B | 120 | 43.890 | −99.706 | −35.254 | 1.00 | 51.10 | C |
| ATOM | 5903 | O | CYS | B | 120 | 44.731 | −98.844 | −34.990 | 1.00 | 50.10 | O |
| ATOM | 5904 | N | LYS | B | 121 | 42.827 | −99.459 | −36.010 | 1.00 | 48.38 | N |
| ATOM | 5905 | CA | LYS | B | 121 | 42.618 | −98.108 | −36.546 | 1.00 | 53.69 | C |
| ATOM | 5906 | CB | LYS | B | 121 | 42.481 | −98.106 | −38.074 | 1.00 | 52.78 | C |
| ATOM | 5907 | CG | LYS | B | 121 | 43.754 | −98.420 | −38.850 | 1.00 | 58.61 | C |
| ATOM | 5908 | CD | LYS | B | 121 | 44.945 | −97.533 | −38.514 | 1.00 | 55.17 | C |
| ATOM | 5909 | CE | LYS | B | 121 | 46.198 | −98.107 | −39.175 | 1.00 | 57.69 | C |
| ATOM | 5910 | NZ | LY5 | B | 121 | 47.343 | −97.256 | −39.225 | 1.00 | 57.73 | N |
| ATOM | 5911 | C | LYS | B | 121 | 41.451 | −97.557 | −35.892 | 1.00 | 56.87 | C |
| ATOM | 5912 | O | LYS | B | 121 | 40.291 | −97.830 | −35.880 | 1.00 | 50.93 | O |
| ATOM | 5915 | N | TYR | B | 122 | 41.777 | −96.282 | −35.350 | 1.00 | 58.95 | N |
| ATOM | 5914 | CA | TYR | B | 122 | 40.790 | −95.556 | −34.659 | 1.00 | 56.16 | C |
| ATOM | 5915 | CB | TYR | B | 122 | 41.414 | −94.649 | −33.461 | 1.00 | 55.10 | C |
| ATOM | 5916 | CG | TYR | B | 122 | 41.946 | −95.592 | −32.400 | 1.00 | 59.30 | C |
| ATOM | 5917 | CD1 | TYR | B | 122 | 41.073 | −96.518 | −31.573 | 1.00 | 58.52 | C |
| ATOM | 5918 | CE1 | TYR | B | 122 | 41.561 | −97.173 | −30.587 | 1.00 | 59.96 | C |
| ATOM | 5919 | CZ | TYR | B | 122 | 42.948 | −97.303 | −30.413 | 1.00 | 63.52 | C |
| ATOM | 5920 | OH | TYR | B | 122 | 43.459 | −98.351 | −29.449 | 1.00 | 62.44 | O |
| ATOM | 5921 | CE2 | TYR | B | 122 | 43.827 | −96.583 | −31214 | 1.00 | 61.78 | C |
| ATOM | 5922 | CD2 | TYR | B | 122 | 43.325 | −95.137 | −32.200 | 1.00 | 59.48 | C |
| ATOM | 5923 | C | TYR | B | 122 | 40.186 | −94.569 | −35.637 | 1.00 | 51.78 | C |
| ATOM | 5924 | O | TYR | B | 122 | 40.628 | −93.220 | −35.732 | 1.00 | 48.27 | O |
| ATOM | 5925 | N | GLU | B | 123 | 39.172 | −94.642 | −36.350 | 1.00 | 50.95 | N |
| ATOM | 5926 | CA | GLU | B | 123 | 38.625 | −94.305 | −37.483 | 1.00 | 54.01 | C |
| ATOM | 5927 | CB | GLU | B | 123 | 38.788 | −94.531 | −38.753 | 1.00 | 56.70 | C |
| ATOM | 5928 | CG | GLU | B | 123 | 40.105 | −94.689 | −39.458 | 1.00 | 59.72 | C |
| ATOM | 5929 | CD | GLU | B | 123 | 40.177 | −93.296 | −40.021 | 1.00 | 66.43 | C |
| ATOM | 5930 | OE1 | GLU | B | 123 | 39.182 | −92.656 | −40.656 | 1.00 | 73.64 | O |
| ATOM | 5931 | OE2 | GLU | B | 123 | 41.222 | −92.647 | −39.822 | 1.00 | 66.29 | O |
| ATOM | 5932 | C | GLU | B | 123 | 37.164 | −93.654 | −37.325 | 1.00 | 50.95 | C |
| ATOM | 5933 | O | GLU | B | 123 | 36.751 | −92.664 | −37.945 | 1.00 | 43.25 | O |
| ATOM | 5934 | N | HIS | B | 124 | 36.401 | −94.404 | −36.523 | 1.00 | 50.98 | N |
| ATOM | 5935 | CA | HIS | B | 124 | 35.011 | −94.075 | −36.209 | 1.00 | 48.95 | C |
| ATOM | 5936 | CB | HIS | B | 124 | 34.189 | −95.340 | −36.030 | 1.00 | 44.71 | C |
| ATOM | 5937 | CG | HIS | B | 124 | 33.731 | −95.964 | −37.315 | 1.00 | 48.97 | C |
| ATOM | 5938 | ND1 | HIS | B | 124 | 32.740 | −95.411 | −38.104 | 1.00 | 47.84 | N |
| ATOM | 5939 | CE1 | HIS | B | 124 | 32.532 | −96.386 | −39.152 | 1.00 | 45.52 | C |
| ATOM | 5940 | NE2 | HIS | B | 124 | 33.342 | −97.229 | −39.068 | 1.00 | 49.14 | N |
| ATOM | 5941 | CD2 | HIS | B | 124 | 34.099 | −97.118 | −37.927 | 1.00 | 46.43 | C |
| ATOM | 5942 | C | HIS | B | 124 | 34.952 | −93.234 | −34.934 | 1.00 | 51.08 | C |
| ATOM | 5943 | O | HIS | B | 124 | 35.802 | −93.374 | −34.040 | 1.00 | 55.37 | O |
| ATOM | 5944 | N | ASP | B | 125 | 33.948 | −92.367 | −34.843 | 1.00 | 28.37 | N |
| ATOM | 5945 | CA | ASP | B | 125 | 33.830 | −91.460 | −33.697 | 1.00 | 28.93 | C |
| ATOM | 5946 | CB | ASP | B | 125 | 33.236 | −90.121 | −34.151 | 1.00 | 49.49 | C |
| ATOM | 5947 | CG | ASP | B | 125 | 34.234 | −89.266 | −34.904 | 1.00 | 51.25 | C |
| ATOM | 5948 | OD1 | ASP | B | 125 | 35.403 | −89.127 | −34.445 | 1.00 | 53.89 | O |
| ATOM | 5949 | OD2 | ASP | B | 125 | 33.838 | −88.714 | −35951 | 1.00 | 50.85 | O |
| ATOM | 5950 | C | ASP | B | 125 | 33.050 | −92.033 | −32.507 | 1.00 | 46.46 | C |
| ATOM | 5951 | O | ASP | B | 123 | 32.949 | −91.401 | −31.462 | 1.00 | 48.61 | O |
| ATOM | 5952 | N | ASP | B | 126 | 32.520 | −93.239 | −32.665 | 1.00 | 46.14 | N |
| ATOM | 5953 | CA | ASP | B | 126 | 31.676 | −93.866 | −31.652 | 1.00 | 46.41 | C |
| ATOM | 5954 | CB | ASP | B | 126 | 30.203 | −93.519 | −31.939 | 1.00 | 46.11 | C |
| ATOM | 5955 | CG | ASP | B | 126 | 29.287 | −93.612 | −30.705 | 1.00 | 46.94 | C |
| ATOM | 5956 | OD1 | ASP | B | 126 | 29.527 | −94.400 | −29.761 | 1.00 | 41.40 | O |
| ATOM | 5957 | OD2 | ASP | B | 126 | 28.267 | −92.884 | −30.708 | 1.00 | 50.52 | O |
| ATOM | 5958 | C | ASP | B | 126 | 31.893 | −95.372 | −31.781 | 1.00 | 47.52 | C |
| ATOM | 5959 | O | ASP | B | 126 | 32.626 | −95.820 | −32.664 | 1.00 | 48.68 | O |
| ATOM | 5960 | N | ILE | B | 127 | 31.253 | −96.147 | −30.911 | 1.00 | 42.98 | N |
| ATOM | 5961 | CA | ILE | B | 127 | 31.255 | −97.589 | −31.005 | 1.00 | 41.68 | C |
| ATOM | 5962 | CB | ILE | B | 127 | 30.104 | −98.193 | −30.158 | 1.00 | 40.68 | C |
| ATOM | 5963 | CG1 | ILE | B | 127 | 30.317 | −97.912 | −28.664 | 1.00 | 40.95 | C |
| ATOM | 5964 | CD1 | ILE | B | 127 | 29.080 | −98.125 | −27.802 | 1.00 | 37.16 | C |
| ATOM | 5965 | CG2 | ILE | B | 127 | 29.983 | −99.685 | −30.391 | 1.00 | 40.34 | C |
| ATOM | 5966 | C | ILE | B | 127 | 31.085 | −98.034 | −32.462 | 1.00 | 43.52 | C |
| ATOM | 5967 | O | ILE | B | 127 | 30.13 | −97.612 | −33.136 | 1.00 | 44.08 | O |
| ATOM | 5968 | N | VAL | B | 128 | 32.011 | −98.876 | −320943 | 1.00 | 41.21 | N |
| ATOM | 5969 | CA | VAL | B | 128 | 31.821 | −99.595 | −34.197 | 1.00 | 37.73 | C |
| ATOM | 5970 | CB | VAL | B | 128 | 33.141 | −100.076 | −34.826 | 1.00 | 40.52 | C |
| ATOM | 5971 | CG1 | VAL | B | 128 | 32.896 | −100.614 | −36.242 | 1.00 | 36.97 | C |
| ATOM | 5972 | CG2 | VAL | B | 128 | 34.187 | −98.958 | −34.846 | 1.00 | 37.98 | C |
| ATOM | 5973 | C | VAL | B | 128 | 30.953 | −100.791 | −33.856 | 1.00 | 42.83 | C |
| ATOM | 5974 | O | VAL | B | 128 | 31.312 | −101.595 | −32.993 | 1.00 | 43.17 | O |
| ATOM | 5975 | N | SER | B | 129 | 29.806 | −100.905 | −34.525 | 1.00 | 43.61 | N |
| ATOM | 5976 | CA | SER | B | 129 | 28.770 | 101.858 | −34.120 | 1.00 | 45.09 | C |
| ATOM | 5977 | CB | SER | B | 129 | 27.422 | −101.136 | −34.033 | 1.00 | 42.52 | C |
| ATOM | 5978 | OG | SER | B | 129 | 27.124 | −100.568 | −35.290 | 1.00 | 41.63 | O |
| ATOM | 5979 | C | SER | B | 129 | 28.629 | −103.031 | −35.069 | 1.00 | 45.97 | C |
| ATOM | 5980 | O | SER | B | 129 | 78.012 | −104.043 | −34.723 | 1.00 | 47.68 | O |
| ATOM | 5981 | N | THR | B | 130 | 29.165 | −102.887 | 36.280 | 1.00 | 48.86 | N |
| ATOM | 5982 | CA | THR | B | 130 | 29.051 | −103.941 | −37.301 | 1.00 | 47.09 | C |
| ATOM | 5983 | CB | THR | B | 130 | 27.693 | −103.890 | −38.035 | 1.00 | 45.69 | C |
| ATOM | 5984 | OG1 | THR | B | 130 | 27.663 | −104.884 | −39.071 | 1.00 | 47.69 | O |
| ATOM | 5985 | CG2 | THR | B | 130 | 27.436 | −102.508 | −38.626 | 1.00 | 41.63 | C |
| ATOM | 5986 | C | THR | B | 130 | 30.216 | −103.928 | −38.315 | 1.00 | 48.00 | C |
| ATOM | 5987 | O | THR | B | 130 | 30.688 | −102.848 | −38.700 | 1.00 | 44.51 | O |
| ATOM | 5988 | N | VAL | B | 131 | 30.675 | −105.134 | −38.696 | 1.00 | 43.77 | N |
| ATOM | 5989 | CA | VAL | B | 131 | 31.618 | −105.335 | −39.805 | 1.00 | 42.09 | C |
| ATOM | 5990 | CB | VAL | B | 131 | 33.097 | −105.509 | −39.356 | 1.00 | 38.74 | C |
| ATOM | 5991 | CG1 | VAL | B | 131 | 33.615 | −104.244 | −38.726 | 1.00 | 35.06 | C |
| ATOM | 5992 | CG2 | VAL | B | 131 | 33.273 | −106.703 | −38.416 | 1.00 | 36.71 | C |
| ATOM | 5993 | C | VAL | B | 131 | 31.246 | −106.542 | −40.662 | 1.00 | 43.96 | C |
| ATOM | 5994 | O | VAL | B | 131 | 30.689 | −107.523 | −40.168 | 1.00 | 41.95 | O |
| ATOM | 5995 | N | SER | B | 132 | 31.574 | −106.450 | −41.947 | 1.00 | 45.06 | N |
| ATOM | 5996 | CA | SER | B | 132 | 31.426 | −107.554 | −42.883 | 1.00 | 49.46 | C |
| ATOM | 5997 | CB | SER | B | 132 | 30.168 | −107.399 | −43.723 | 1.00 | 48.28 | C |
| ATOM | 5998 | OG | SER | B | 132 | 29.634 | −108.671 | −44.034 | 1.00 | 54.33 | O |
| ATOM | 5999 | C | SER | B | 132 | 32.637 | −107.546 | −43.804 | 1.00 | 52.32 | C |
| ATOM | 6000 | O | SER | B | 132 | 33.179 | −106.467 | −44.110 | 1.00 | 51.62 | O |
| ATOM | 6001 | N | VAL | B | 133 | 33.071 | −108.738 | −44.227 | 1.00 | 51.06 | N |
| ATOM | 6002 | CA | VAL | B | 133 | 34.168 | −108.837 | −45.191 | 1.00 | 52.84 | C |
| ATOM | 6003 | CB | VAL | B | 133 | 35.118 | −110.053 | −44.982 | 1.00 | 54.30 | C |
| ATOM | 6004 | CG1 | VAL | B | 133 | 36.534 | −109.693 | −45.411 | 1.00 | 54.47 | C |
| ATOM | 6005 | CG2 | VAL | B | 133 | 35.139 | −110.524 | −43.547 | 1.00 | 52.45 | C |
| ATOM | 6006 | C | VAL | B | 133 | 33.548 | −108.967 | −46.565 | 1.00 | 48.96 | C |
| ATOM | 6007 | O | VAL | B | 133 | 32.621 | −109.765 | −46.766 | 1.00 | 45.51 | O |
| ATOM | 6008 | N | LEU | B | 134 | 34.072 | −108.181 | −47.499 | 1.00 | 48.68 | N |
| ATOM | 6009 | CA | LEU | B | 134 | 33.616 | −108.117 | −48.889 | 1.00 | 54.82 | C |
| ATOM | 6010 | CB | LEU | B | 134 | 33.960 | −106.194 | −49.584 | 1.00 | 54.90 | C |
| ATOM | 6011 | CG | LEU | B | 134 | 33.160 | −105.585 | −49.061 | 1.00 | 54.13 | C |
| ATOM | 6012 | CD1 | LEU | B | 334 | 33.856 | −104.350 | −49.311 | 1.00 | 50.36 | C |
| ATOM | 6013 | CD2 | LEU | B | 134 | 31.760 | −105.577 | −49.656 | 1.00 | 51.36 | C |
| ATOM | 6014 | C | LEU | B | 134 | 34.142 | −109.169 | −49.643 | 1.00 | 56.89 | C |
| ATOM | 6015 | O | LEU | B | 134 | 35.128 | −110.195 | −49.222 | 1.00 | 59.17 | O |
| ATOM | 6016 | N | SER | B | 135 | 33.467 | −109.132 | −50.736 | 1.00 | 59.13 | N |
| ATOM | 6017 | CA | SER | B | 135 | 33.615 | −111.166 | −51.364 | 1.00 | 60.73 | C |
| ATOM | 6018 | CB | SER | B | 135 | 32.712 | −111.121 | −52.599 | 1.00 | 56.47 | C |
| ATOM | 6019 | OG | SER | B | 135 | 32.997 | −110.111 | −53.561 | 1.00 | 57.62 | O |
| ATOM | 6020 | C | SER | B | 135 | 35.061 | −111.182 | −51.678 | 1.00 | 58.42 | C |
| ATOM | 6021 | O | SER | B | 135 | 35.455 | −112.105 | −51.362 | 1.00 | 56.01 | O |
| ATOM | 6022 | N | SER | B | 136 | 35.845 | −110.575 | −52.260 | 1.00 | 57.69 | N |
| ATOM | 6023 | CA | SER | B | 136 | 37.247 | −110.166 | −52.579 | 1.00 | 62.44 | C |
| ATOM | 6024 | CB | SER | 8 | 136 | 37.958 | −109.143 | −53.158 | 1.00 | 63.02 | C |
| ATOM | 6025 | OG | SER | B | 136 | 38.527 | −108.356 | −52.123 | 1.00 | 65.44 | O |
| ATOM | 6026 | C | SER | B | 136 | 38.022 | −111.169 | −51.357 | 1.00 | 62.61 | C |
| ATOM | 6027 | O | SER | B | 136 | 38.788 | −112.429 | −51.473 | 1.00 | 66.69 | O |
| ATOM | 6028 | N | GLY | B | 137 | 37.800 | −110.125 | −50.206 | 1.00 | 55.48 | N |
| ATOM | 6029 | CA | GLY | B | 137 | 38.539 | −111.087 | −48.974 | 1.00 | 47.50 | C |
| ATOM | 6030 | C | GLY | B | 137 | 39.581 | −109.196 | −48.716 | 1.00 | 49.55 | C |
| ATOM | 6031 | O | GLY | B | 337 | 40.368 | −110.080 | −47.762 | 1.00 | 45.40 | O |
| ATOM | 6032 | N | THR | B | 338 | 39.598 | −108.171 | −49.565 | 1.00 | 49.24 | N |
| ATOM | 6033 | CA | THR | B | 138 | 40.621 | −107.122 | −49.471 | 1.00 | 53.73 | C |
| ATOM | 6034 | CB | THR | B | 138 | 41.237 | −107.632 | −50.849 | 1.00 | 54.31 | C |
| ATOM | 6035 | OG1 | THR | B | 138 | 40.189 | −107.141 | −51.786 | 1.00 | 53.93 | O |
| ATOM | 6036 | CG2 | THR | B | 138 | 42.044 | −108.128 | −51.340 | 1.00 | 52.84 | C |
| ATOM | 6037 | C | THR | B | 138 | 40.094 | −106.403 | −48.899 | 1.00 | 60.37 | C |
| ATOM | 6038 | O | THR | B | 138 | 40.873 | −105.690 | −48.571 | 1.00 | 59.73 | O |
| ATOM | 6039 | N | GLN | B | 138 | 38.767 | −106.505 | −48.805 | 1.00 | 60.19 | N |
| ATOM | 6040 | CA | GLN | B | 139 | 38.104 | −105.110 | −48.280 | 1.00 | 58.93 | C |
| ATOM | 6041 | CB | GLN | B | 139 | 37.533 | −104.184 | −49.426 | 1.00 | 60.43 | C |
| ATOM | 6042 | CG | GLN | B | 139 | 38.567 | −104.017 | −50.436 | 1.00 | 62.28 | C |
| ATOM | 6043 | CD | GLN | 8 | 139 | 37.938 | −103.163 | −51.581 | 1.00 | 62.13 | C |
| ATOM | 6044 | OE1 | GLN | B | 139 | 36.830 | −103.179 | −52.015 | 1.00 | 65.81 | O |
| ATOM | 6045 | NE2 | GLN | B | 139 | 38.637 | −102.154 | −52.073 | 1.00 | 64.61 | N |
| ATOM | 6046 | C | GLN | B | 139 | 36.996 | −105.412 | −47.262 | 1.00 | 55.88 | C |
| ATOM | 6047 | O | GLN | B | 139 | 36.443 | −106.123 | −47.205 | 1.00 | 52.09 | O |
| ATOM | 6048 | N | ALA | B | 140 | 36.673 | −104.608 | −46.459 | 1.00 | 52.32 | N |
| ATOM | 6049 | CA | ALA | B | 140 | 35.563 | −104.124 | −45.525 | 1.00 | 50.26 | C |
| ATOM | 6050 | CB | ALA | B | 140 | 36.039 | −105.166 | −44.186 | 1.00 | 49.35 | C |
| ATOM | 6051 | C | ALA | B | 140 | 34.82 | −103.412 | −45.342 | 1.00 | 48.76 | C |
| ATOM | 6052 | O | ALA | B | 140 | 35.386 | −102.133 | −45.508 | 1.00 | 46.92 | O |
| ATOM | 6053 | N | VAL | B | 141 | 33.535 | −103.133 | −45.017 | 1.00 | 52.43 | N |
| ATOM | 6054 | CA | VAL | B | 141 | 32.678 | −102.192 | −44.654 | 1.00 | 45.01 | C |
| ATOM | 6055 | CB | VAL | B | 141 | 31.412 | −102.103 | −45.538 | 1.00 | 45.75 | C |
| ATOM | 6056 | CG1 | VAL | B | 141 | 30.82 | −103.679 | −45.835 | 1.00 | 44.54 | C |
| ATOM | 6057 | CG2 | VAL | B | 141 | 30.363 | −101.431 | −44.876 | 1.00 | 48.04 | C |
| ATOM | 6058 | C | VAL | B | 141 | 32.311 | −102.514 | −43.175 | 1.00 | 40.96 | C |
| ATOM | 6059 | O | VAL | B | 141 | 31.986 | −103.101 | −42.697 | 1.00 | 40.51 | O |
| ATOM | 6060 | N | SER | B | 142 | 32.417 | −101.404 | −42.456 | 1.00 | 38.48 | N |
| ATOM | 6061 | CA | SER | B | 142 | 32.033 | −101.326 | −41.053 | 1.00 | 36.47 | C |
| ATOM | 6062 | CB | SER | B | 142 | 33.218 | −100.126 | −40.234 | 1.00 | 36.60 | C |
| ATOM | 6063 | OG | SER | B | 142 | 33.728 | −99.627 | −40.787 | 1.00 | 31.80 | O |
| ATOM | 6064 | C | SER | B | 142 | 30.887 | −100.326 | −40.899 | 1.00 | 41.75 | C |
| ATOM | 6065 | O | SER | B | 142 | 30.750 | −99.393 | −41.723 | 1.00 | 38.85 | O |
| ATOM | 6066 | N | GLY | B | 143 | 30.091 | −100.523 | −39.838 | 1.00 | 44.44 | N |
| ATOM | 6067 | CA | GLY | B | 143 | 29.039 | −99.592 | −39.418 | 1.00 | 43.04 | C |
| ATOM | 6068 | C | GLY | B | 143 | 29.236 | −99.117 | −37.993 | 1.00 | 44.12 | C |
| ATOM | 6069 | O | GLY | B | 143 | 29.619 | −99.192 | −37.114 | 1.00 | 46.75 | O |
| ATOM | 6070 | N | SER | B | 144 | 28.973 | −97.840 | −37.751 | 1.00 | 46.78 | N |
| ATOM | 6071 | CA | SER | B | 144 | 29.108 | −97.274 | −36.395 | 1.00 | 47.89 | C |
| ATOM | 6072 | CB | SER | B | 144 | 30.298 | −96.307 | −36.368 | 1.00 | 45.29 | C |
| ATOM | 6073 | OG | SER | B | 144 | 30.465 | −95.674 | −35.109 | 1.00 | 46.89 | O |
| ATOM | 6074 | C | SER | B | 144 | 27.823 | −96.578 | −35.869 | 1.00 | 46.98 | C |
| ATOM | 6075 | O | SER | B | 144 | 26.939 | −96.204 | −36.644 | 1.00 | 46.40 | O |
| ATOM | 6076 | N | LYS | B | 145 | 27.739 | −96.422 | −34.550 | 1.00 | 45.97 | N |
| ATOM | 6077 | CA | LYS | B | 145 | 26.802 | −95.489 | −33.927 | 1.00 | 46.69 | C |
| ATOM | 6078 | CB | LYS | B | 145 | 26.820 | −95.606 | −32.402 | 1.00 | 45.76 | C |
| ATOM | 6079 | CD | LYS | B | 145 | 26.431 | −96.986 | −31.891 | 1.00 | 47.74 | C |
| ATOM | 6080 | CD | LYS | B | 145 | 25.876 | −96.941 | −30.473 | 1.00 | 46.93 | C |
| ATOM | 6081 | CE | LYS | B | 145 | 25.606 | −98.336 | −29.919 | 1.00 | 46.68 | C |
| ATOM | 6082 | NZ | LYS | B | 145 | 24.527 | −99.041 | −30.668 | 1.00 | 50.60 | N |
| ATOM | 6083 | C | LYS | B | 145 | 27.057 | −94.036 | −34.349 | 1.00 | 47.05 | C |
| ATOM | 6084 | O | LYS | B | 145 | 26.179 | −93.184 | −34.174 | 1.00 | 51.38 | O |
| ATOM | 6085 | N | ASP | B | 146 | 28.235 | −93.760 | −34.920 | 1.00 | 43.73 | N |
| ATOM | 6086 | CA | ASP | B | 146 | 28.580 | −92.414 | −35.400 | 1.00 | 43.09 | C |
| ATOM | 6087 | CB | ASP | B | 146 | 30.108 | −92.212 | −35.473 | 1.00 | 48.38 | C |
| ATOM | 6088 | CG | ASP | B | 146 | 30.755 | −92.842 | −36.716 | 1.00 | 46.54 | C |
| ATOM | 6089 | OD1 | ASP | B | 146 | 30.141 | −93.668 | −37.428 | 1.00 | 42.69 | O |
| ATOM | 6090 | OD2 | ASP | B | 146 | 31.913 | −92.484 | −36.978 | 1.00 | 51.98 | O |
| ATOM | 6091 | C | ASP | B | 146 | 27.919 | −92.094 | −36.748 | 1.00 | 44.51 | C |
| ATOM | 6092 | O | ASP | B | 146 | 28.215 | −91.065 | −37.376 | 1.00 | 40.46 | O |
| ATOM | 6093 | N | ILE | B | 147 | 27.023 | −92.996 | −37.365 | 1.00 | 43.50 | N |
| ATOM | 6094 | CA | ILE | B | 147 | 26.183 | −92.866 | −38.363 | 1.00 | 44.54 | C |
| ATOM | 6095 | CB | ILE | B | 147 | 25.351 | −91.553 | −38.339 | 1.00 | 43.56 | C |
| ATOM | 6096 | CG1 | ILE | B | 147 | 24.704 | −91.388 | −36.951 | 1.00 | 45.02 | C |
| ATOM | 6097 | CD1 | ILE | B | 147 | 24.160 | −89.994 | −36.637 | 1.00 | 38.58 | C |
| ATOM | 6098 | CG2 | ILE | B | 147 | 24.261 | −91.584 | −39.406 | 1.00 | 42.12 | C |
| ATOM | 6099 | C | ILE | B | 147 | 26.945 | −93.137 | −39.696 | 1.00 | 46.06 | C |
| ATOM | 6100 | O | ILE | B | 147 | 26.347 | −93.178 | −40.772 | 1.00 | 43.95 | O |
| ATOM | 6101 | N | CYS | B | 148 | 28.259 | −93.363 | −39.609 | 1.00 | 50.83 | N |
| ATOM | 0102 | CA | CYS | B | 148 | 29.087 | −95.656 | −40.799 | 1.00 | 47.32 | C |
| ATOM | 6103 | CB | CYS | B | 148 | 30.450 | −92.968 | −40.666 | 1.00 | 47.77 | C |
| ATOM | 6104 | SG | CYS | B | 148 | 30.257 | −91.178 | −40.611 | 1.00 | 54.93 | S |
| ATOM | 6105 | C | CYS | B | 148 | 29.225 | −95.110 | −41.180 | 1.00 | 45.70 | C |
| ATOM | 6106 | O | CYS | B | 148 | 28.991 | −96.006 | −40.370 | 1.00 | 52.38 | O |
| ATOM | 6107 | N | ILE | B | 149 | 29.563 | −95.333 | −42.446 | 1.00 | 45.22 | N |
| ATOM | 6108 | CA | ILE | B | 149 | 29.784 | −96.643 | −43.038 | 1.00 | 41.76 | C |
| ATOM | 6109 | CB | ILE | B | 149 | 28.638 | −97.038 | −43.986 | 1.00 | 40.55 | C |
| ATOM | 6110 | CG1 | ILE | B | 149 | 27.402 | −97.430 | −43.185 | 1.00 | 43.10 | C |
| ATOM | 6111 | CD1 | ILE | B | 149 | 26.156 | −97.752 | −44.014 | 1.00 | 42.94 | C |
| ATOM | 6112 | CG2 | ILE | B | 149 | 29.026 | −98.215 | −44.862 | 1.00 | 41.80 | C |
| ATOM | 6113 | C | ILE | B | 149 | 31.084 | −96.441 | −43.795 | 1.00 | 45.49 | C |
| ATOM | 6114 | O | ILE | B | 149 | 31.242 | −95.436 | −44.491 | 1.00 | 41.71 | O |
| ATOM | 6115 | N | LYS | B | 150 | 32.044 | −97.348 | −43.601 | 1.00 | 49.26 | N |
| ATOM | 6116 | CA | LYS | B | 150 | 33.407 | −97.131 | −44.108 | 1.00 | 43.72 | C |
| ATOM | 6117 | CB | LYS | B | 150 | 34.369 | −96.788 | −42.967 | 1.00 | 43.39 | C |
| ATOM | 6118 | CG | LYS | B | 150 | 34.202 | −95.404 | −42.377 | 1.00 | 43.07 | C |
| ATOM | 6119 | CD | LYS | B | 150 | 35.273 | −95.143 | −41.333 | 1.00 | 44.63 | C |
| ATOM | 6120 | CE | LYS | B | 150 | 34.890 | −94.024 | −40.382 | 1.00 | 41.66 | C |
| ATOM | 6121 | NZ | LYS | B | 150 | 35.047 | −92.686 | −41.006 | 1.00 | 41.75 | N |
| ATOM | 6122 | C | LYS | B | 150 | 33.942 | −98.328 | −44.880 | 1.00 | 40.53 | C |
| ATOM | 6123 | O | LYS | B | 150 | 33.746 | −99.459 | −44.482 | 1.00 | 48.04 | O |
| ATOM | 6124 | N | VAL | B | 151 | 34.620 | −98.066 | −45.988 | 1.00 | 50.84 | N |
| ATOM | 6125 | CA | VAL | B | 151 | 35.180 | −99.150 | −46.793 | 1.00 | 51.76 | C |
| ATOM | 6126 | CB | VAL | B | 151 | 34.811 | −99.007 | −48.280 | 1.00 | 57.68 | C |
| ATOM | 6127 | CG1 | VAL | B | 151 | 35.560 | −100.037 | −49.116 | 1.00 | 60.72 | C |
| ATOM | 6128 | CG2 | VAL | B | 151 | 33.307 | −99.174 | −48.461 | 1.00 | 54.29 | C |
| ATOM | 6129 | C | VAL | B | 151 | 36.692 | −99.263 | −46.584 | 1.00 | 54.46 | C |
| ATOM | 6130 | O | VAL | B | 151 | 37.446 | −98.300 | −46.760 | 1.00 | 52.05 | O |
| ATOM | 6131 | N | TRP | B | 152 | 37.119 | −100.451 | −46.191 | 1.00 | 54.80 | N |
| ATOM | 6132 | CA | TRP | B | 152 | 38.498 | −100.665 | −45.785 | 1.00 | 61.73 | C |
| ATOM | 6133 | CB | TRP | B | 152 | 38.511 | −101.452 | −44.481 | 1.00 | 60.43 | C |
| ATOM | 6134 | CG | TRP | B | 152 | 37.952 | −100.694 | −43.328 | 1.00 | 61.25 | C |
| ATOM | 6135 | CD1 | TRP | B | 152 | 36.638 | −100.596 | −42.963 | 1.00 | 60.17 | C |
| ATOM | 6136 | NE1 | TRP | B | 152 | 36.516 | −99.815 | −41.841 | 1.00 | 60.54 | N |
| ATOM | 6137 | CE2 | TRP | B | 152 | 37.762 | −99.391 | −41.458 | 1.00 | 63.82 | C |
| ATOM | 6138 | CD2 | TRP | B | 152 | 38.692 | −99.923 | −42.379 | 1.00 | 62.04 | C |
| ATOM | 6139 | CE3 | TRP | B | 152 | 40.055 | −99.640 | −42.208 | 1.00 | 60.02 | C |
| ATOM | 6140 | CZ3 | TRP | B | 152 | 40.441 | −98.841 | −41.137 | 1.00 | 61.80 | C |
| ATOM | 6141 | CH2 | TRP | B | 152 | 39.491 | −98.322 | −40.239 | 1.00 | 62.30 | C |
| ATOM | 6142 | CZ2 | TRP | B | 152 | 38.152 | −98.583 | −40.381 | 1.00 | 62.33 | C |
| ATOM | 6143 | C | TRP | B | 152 | 39.320 | −101.416 | −46.825 | 1.00 | 61.60 | C |
| ATOM | 6144 | O | TRP | B | 152 | 38.827 | −102.369 | −47.435 | 1.00 | 62.66 | O |
| ATOM | 6145 | N | ASP | B | 153 | 40.561 | −100.973 | −47.022 | 1.00 | 62.63 | N |
| ATOM | 6146 | CA | ASP | B | 153 | 41.602 | −101.788 | −47.669 | 1.00 | 65.57 | C |
| ATOM | 6147 | CB | ASP | B | 153 | 42.548 | −100.910 | −48.494 | 1.00 | 74.26 | C |
| ATOM | 6148 | CG | ASP | B | 153 | 43.431 | −101.718 | −49.447 | 1.00 | 81.30 | C |
| ATOM | 6149 | OD1 | ASP | B | 153 | 43.566 | −102.950 | −49.268 | 1.00 | 80.65 | O |
| ATOM | 6150 | OD2 | ASP | B | 153 | 43.994 | −101.106 | −50.383 | 1.00 | 81.57 | O |
| ATOM | 6151 | C | ASP | B | 153 | 42.403 | −102.540 | −46.595 | 1.00 | 59.52 | C |
| ATOM | 6152 | O | ASP | B | 153 | 43.207 | −101.937 | −45.867 | 1.00 | 53.43 | O |
| ATOM | 6153 | N | LEU | B | 154 | 42.177 | −103.850 | −46.497 | 1.00 | 56.41 | N |
| ATOM | 6154 | CA | LEU | B | 154 | 42.777 | −104.659 | −45.415 | 1.00 | 58.59 | C |
| ATOM | 6155 | CB | LEU | B | 154 | 42.110 | −106.023 | −45.303 | 1.00 | 52.80 | C |
| ATOM | 6156 | CG | LEU | B | 154 | 40.614 | −105.884 | −45.027 | 1.00 | 52.72 | C |
| ATOM | 6157 | CD1 | LEU | B | 154 | 39.918 | −157.219 | −45.234 | 1.00 | 50.38 | C |
| ATOM | 6158 | CD2 | LEU | B | 154 | 40.371 | −105.313 | −43.626 | 1.00 | 51.68 | C |
| ATOM | 6159 | C | LEU | B | 154 | 44.277 | −104.837 | −45.547 | 1.00 | 64.03 | C |
| ATOM | 6160 | O | LEU | B | 154 | 44.991 | −104.856 | −44.539 | 1.00 | 62.46 | O |
| ATOM | 6161 | N | ALA | B | 155 | 44.738 | −104.975 | −46.792 | 1.00 | 63.98 | N |
| ATOM | 6162 | CA | ALA | B | 155 | 46.159 | −105.010 | −47.090 | 1.00 | 63.87 | C |
| ATOM | 6163 | CB | ALA | B | 155 | 46.391 | −105.538 | −48.562 | 1.00 | 63.13 | C |
| ATOM | 6164 | C | ALA | B | 155 | 46.800 | −103.675 | −46.715 | 1.00 | 60.36 | C |
| ATOM | 6165 | O | ALA | B | 155 | 47.812 | −103.649 | −46.021 | 1.00 | 58.65 | O |
| ATOM | 6166 | N | GLN | B | 156 | 46.198 | −102.570 | −47.152 | 1.00 | 62.37 | N |
| ATOM | 6167 | CA | GLN | B | 156 | 46.770 | −101.245 | −46.896 | 1.00 | 65.31 | C |
| ATOM | 6168 | CB | GLN | B | 156 | 46.335 | −100.234 | −47.973 | 1.00 | 68.55 | C |
| ATOM | 6169 | CG | GLN | B | 156 | 47.229 | −100.209 | −49.209 | 1.00 | 73.69 | C |
| ATOM | 6170 | CD | GLN | B | 156 | 48.703 | −99.995 | −48.866 | 1.00 | 73.90 | C |
| ATOM | 6171 | OE1 | GLN | B | 156 | 49.112 | −98.893 | −48.491 | 1.00 | 69.89 | O |
| ATOM | 6172 | NE2 | GLN | B | 156 | 49.504 | −101.657 | −48.986 | 1.00 | 72.34 | N |
| ATOM | 6173 | C | GLN | B | 156 | 46.520 | −100.682 | −45.488 | 1.00 | 64.49 | C |
| ATOM | 6174 | O | GLN | B | 156 | 47.181 | −99.721 | −45.082 | 1.00 | 62.29 | O |
| ATOM | 6175 | N | GLN | B | 157 | 45.580 | −101.277 | −44.746 | 1.00 | 64.31 | N |
| ATOM | 6176 | CA | GLN | B | 157 | 45.206 | −100.773 | −43.414 | 1.00 | 63.92 | C |
| ATOM | 6177 | CB | GLN | B | 157 | 46.379 | −100.898 | −42.431 | 1.00 | 65.04 | C |
| ATOM | 6178 | CG | GLN | B | 157 | 47.063 | −102.263 | −42.436 | 1.00 | 62.42 | C |
| ATOM | 6179 | CD | GLN | B | 157 | 48.124 | −102.384 | −41.363 | 1.00 | 57.86 | C |
| ATOM | 6180 | OE1 | GLN | B | 157 | 48.578 | −101.384 | −40.800 | 1.00 | 55.85 | O |
| ATOM | 6181 | NE2 | GLN | B | 157 | 48.522 | −103.613 | −41.069 | 1.00 | 57.89 | N |
| ATOM | 6182 | C | GLN | B | 157 | 44.717 | −99.310 | −43.469 | 1.00 | 63.11 | C |
| ATOM | 6183 | O | GLN | B | 157 | 45.123 | −98.474 | −42.648 | 1.00 | 64.49 | O |
| ATOM | 6184 | N | VAL | B | 158 | 43.878 | −99.101 | −44.459 | 1.00 | 56.09 | N |
| ATOM | 6185 | CA | VAL | B | 158 | 43.271 | −97.681 | −44.558 | 1.00 | 54.26 | C |
| ATOM | 6186 | CB | VAL | B | 158 | 44.046 | −96.721 | −45.508 | 1.00 | 53.74 | C |
| ATOM | 6187 | CG1 | VAL | B | 158 | 45.333 | −96.201 | −44.872 | 1.00 | 51.59 | C |
| ATOM | 6188 | CG2 | VAL | B | 158 | 44.279 | −97.359 | −46.867 | 1.00 | 50.43 | C |
| ATOM | 6189 | C | VAL | B | 158 | 41.797 | −97.703 | −44.978 | 1.00 | 52.41 | C |
| ATOM | 6190 | O | VAL | B | 158 | 41.304 | −98.677 | −45.563 | 1.00 | 50.30 | O |
| ATOM | 6191 | N | VAL | B | 159 | 41.124 | −96.594 | −44.679 | 1.00 | 53.74 | N |
| ATOM | 6192 | CA | VAL | B | 159 | 39.772 | −96.302 | −45.153 | 1.00 | 52.48 | C |
| ATOM | 6193 | CB | VAL | B | 159 | 39.078 | −95.240 | −44.259 | 1.00 | 50.72 | C |
| ATOM | 6194 | CG1 | VAL | B | 159 | 37.690 | −94.879 | −44.794 | 1.00 | 48.98 | C |
| ATOM | 6195 | CG2 | VAL | B | 159 | 38.993 | −95.719 | −42.818 | 1.00 | 51.43 | C |
| ATOM | 6196 | C | VAL | B | 159 | 39.873 | −95.728 | −46.554 | 1.00 | 53.78 | C |
| ATOM | 6197 | O | VAL | B | 159 | 40.491 | −94.677 | −46.748 | 1.00 | 54.13 | O |
| ATOM | 6198 | N | LEU | B | 160 | 39.256 | −96.408 | −47.519 | 1.00 | 54.53 | N |
| ATOM | 6199 | CA | LEU | B | 160 | 39.197 | −95.915 | −48.895 | 1.00 | 58.93 | C |
| ATOM | 6200 | CB | LEU | B | 160 | 39.090 | −97.078 | −49.894 | 1.00 | 57.12 | C |
| ATOM | 6201 | CG | LEU | B | 160 | 40.119 | −98.215 | −49.818 | 1.00 | 59.31 | C |
| ATOM | 6202 | CD1 | LEU | B | 160 | 39.488 | −99.544 | −50.195 | 1.00 | 57.74 | C |
| ATOM | 6203 | CD2 | LEU | B | 160 | 41.364 | −97.949 | −50.657 | 1.00 | 59.81 | C |
| ATOM | 6204 | C | LEU | B | 160 | 38.013 | −94.958 | −49.058 | 1.00 | 63.48 | C |
| ATOM | 6205 | O | LEU | B | 160 | 38.169 | −93.844 | −49.571 | 1.00 | 65.59 | O |
| ATOM | 6206 | N | SER | B | 161 | 36.841 | −95.400 | −48.604 | 1.00 | 64.09 | N |
| ATOM | 6207 | CA | SER | B | 161 | 35.602 | −94.634 | −48.713 | 1.00 | 62.49 | C |
| ATOM | 6208 | CB | SER | B | 161 | 34.682 | −95.286 | −49.735 | 1.00 | 63.66 | C |
| ATOM | 6209 | OG | SER | B | 161 | 35.126 | −95.027 | −51.047 | 1.00 | 70.60 | O |
| ATOM | 6210 | C | SER | B | 161 | 34.859 | −94.512 | −47.381 | 1.00 | 63.00 | C |
| ATOM | 6211 | O | SER | B | 161 | 34.755 | −95.480 | −46.619 | 1.00 | 63.09 | O |
| ATOM | 6212 | N | SER | B | 162 | 34.339 | −93.315 | −47.124 | 1.00 | 58.54 | N |
| ATOM | 6213 | CA | SER | B | 162 | 33.573 | −93.019 | −45.925 | 1.00 | 54.52 | C |
| ATOM | 6214 | CB | SER | B | 162 | 34.348 | −92.030 | −45.072 | 1.00 | 55.15 | C |
| ATOM | 6215 | OG | SER | B | 162 | 33.851 | −92.024 | −43.753 | 1.00 | 59.41 | O |
| ATOM | 6216 | C | SER | B | 162 | 32.210 | −92.432 | −46.324 | 1.00 | 55.16 | C |
| ATOM | 6217 | O | SER | B | 162 | 32.153 | −91.405 | −47.005 | 1.00 | 52.93 | O |
| ATOM | 6218 | N | TYR | B | 163 | 31.124 | −93.090 | −45.912 | 1.00 | 51.82 | N |
| ATOM | 6219 | CA | TYR | B | 163 | 29.769 | −92.704 | −46.311 | 1.00 | 52.43 | C |
| ATOM | 6220 | CB | TYR | B | 163 | 29.125 | −93.814 | −47.140 | 1.00 | 57.72 | C |
| ATOM | 6221 | CG | TYR | B | 163 | 29.906 | −94.248 | −48.357 | 1.00 | 65.81 | C |
| ATOM | 6222 | CD1 | TYR | B | 163 | 30.048 | −93.405 | −49.459 | 1.00 | 67.45 | C |
| ATOM | 6223 | CE1 | TYR | B | 163 | 30.759 | −93.809 | −50.581 | 1.00 | 72.41 | C |
| ATOM | 6224 | CZ | TYR | B | 163 | 31.332 | −95.076 | −50.616 | 1.00 | 75.73 | C |
| ATOM | 6225 | OH | TYR | B | 163 | 32.047 | −95.485 | −51.729 | 1.00 | 77.61 | O |
| ATOM | 6226 | CE2 | TYR | B | 163 | 31.194 | −95.930 | −49.534 | 1.00 | 71.82 | C |
| ATOM | 6227 | CD2 | TYR | B | 163 | 30.483 | −95.516 | −48.417 | 1.00 | 68.74 | C |
| ATOM | 6228 | C | TYR | B | 163 | 28.823 | −92.366 | −45.143 | 1.00 | 54.27 | C |
| ATOM | 6229 | O | TYR | B | 163 | 28.523 | −93.220 | −44.299 | 1.00 | 53.21 | O |
| ATOM | 6230 | N | ARG | B | 164 | 28.338 | −91.123 | −45.115 | 1.00 | 53.72 | N |
| ATOM | 6231 | CA | ARG | B | 164 | 27.251 | −90.715 | −44.223 | 1.00 | 51.32 | C |
| ATOM | 6232 | CB | ARG | B | 164 | 27.447 | −89.280 | −43.762 | 1.00 | 56.01 | C |
| ATOM | 6233 | CG | ARG | B | 164 | 27.999 | −89.136 | −42.371 | 1.00 | 63.23 | C |
| ATOM | 6234 | CD | ARG | B | 164 | 27.957 | −87.686 | −41.928 | 1.00 | 66.12 | C |
| ATOM | 6235 | NE | ARG | B | 164 | 26.930 | −87.461 | −40.921 | 1.00 | 69.62 | N |
| ATOM | 6236 | CZ | ARG | B | 164 | 27.072 | −87.734 | −39.620 | 1.00 | 73.84 | C |
| ATOM | 6237 | NH1 | ARG | B | 164 | 28.205 | −88.262 | −39.158 | 1.00 | 69.44 | N |
| ATOM | 6238 | NH2 | ARG | B | 164 | 26.071 | −87.485 | −38.777 | 1.00 | 68.43 | N |
| ATOM | 6239 | C | ARG | B | 164 | 25.919 | −90.795 | −44.944 | 1.00 | 48.59 | C |
| ATOM | 6240 | O | ARG | B | 164 | 25.333 | −89.772 | −45.278 | 1.00 | 49.51 | O |
| ATOM | 6241 | N | ALA | B | 165 | 25.438 | −91.999 | −45.202 | 1.00 | 47.62 | N |
| ATOM | 6242 | CA | ALA | B | 165 | 24.182 | −92.140 | −45.919 | 1.00 | 48.24 | C |
| ATOM | 6243 | CB | ALA | B | 165 | 24.125 | −93.472 | −46.640 | 1.00 | 47.18 | C |
| ATOM | 6244 | C | ALA | B | 165 | 23.019 | −92.012 | −44.958 | 1.00 | 45.75 | C |
| ATOM | 6245 | O | ALA | B | 165 | 21.974 | −91.469 | −45.301 | 1.00 | 49.86 | O |
| ATOM | 6246 | N | HIS | B | 166 | 23.234 | −92.502 | −43.746 | 1.00 | 44.18 | N |
| ATOM | 6247 | CA | HIS | B | 166 | 22.179 | −92.673 | −42.777 | 1.00 | 45.25 | C |
| ATOM | 6248 | CB | HIS | B | 166 | 22.488 | −93.888 | −41.925 | 1.00 | 45.90 | C |
| ATOM | 6249 | CG | HIS | B | 166 | 22.209 | −95.182 | −42.611 | 1.00 | 45.12 | C |
| ATOM | 6250 | ND1 | HIS | B | 166 | 20.930 | −95.598 | −42.916 | 1.00 | 44.43 | N |
| ATOM | 6251 | CE1 | HIS | B | 166 | 20.986 | −96.780 | −43.499 | 1.00 | 41.52 | C |
| ATOM | 6252 | NE2 | HIS | B | 166 | 22.255 | −97.146 | −43.579 | 1.00 | 43.05 | N |
| ATOM | 6253 | CD2 | HIS | B | 166 | 23.038 | −96.166 | −43.028 | 1.00 | 43.04 | C |
| ATOM | 6254 | C | HIS | B | 166 | 21.940 | −91.455 | −41.893 | 1.00 | 45.31 | C |
| ATOM | 6255 | O | HIS | B | 166 | 22.728 | −90.509 | −41.908 | 1.00 | 43.18 | O |
| ATOM | 6256 | N | ALA | B | 167 | 20.844 | −91.472 | −41.132 | 1.00 | 45.64 | N |
| ATOM | 6257 | CA | ALA | B | 167 | 20.558 | −90.335 | −40.249 | 1.00 | 51.66 | C |
| ATOM | 6258 | CB | ALA | B | 167 | 19.399 | −89.498 | −40.779 | 1.00 | 43.78 | C |
| ATOM | 6259 | C | ALA | B | 167 | 20.383 | −90.716 | −38.766 | 100 | 52.81 | C |
| ATOM | 6260 | O | ALA | B | 167 | 20.002 | −89.895 | −37.918 | 1.00 | 53.10 | O |
| ATOM | 6261 | N | ALA | B | 168 | 20.695 | −91.975 | −38.483 | 1.00 | 48.67 | N |
| ATOM | 6262 | CA | ALA | B | 168 | 20.783 | −92.492 | −37.133 | 100 | 49.49 | C |
| ATOM | 6263 | CB | ALA | B | 168 | 19.435 | −93.021 | −36.680 | 1.00 | 47.49 | C |
| ATOM | 6264 | C | ALA | B | 168 | 21.836 | −93.606 | −37.138 | 1.00 | 49.98 | C |
| ATOM | 6265 | O | ALA | B | 168 | 22.244 | −94.076 | −38.198 | 1.00 | 53.91 | O |
| ATOM | 6266 | N | GLN | B | 169 | 22.281 | −94.014 | −35.957 | 1.00 | 46.69 | N |
| ATOM | 6267 | CA | GLN | B | 169 | 23.323 | −95.022 | −35.819 | 1.00 | 45.50 | C |
| ATOM | 6268 | CB | GLN | B | 169 | 23.483 | −95.416 | −34.351 | 1.00 | 43.61 | C |
| ATOM | 6269 | CG | GLN | B | 169 | 22.165 | −95.636 | −33.641 | 1.00 | 43.43 | C |
| ATOM | 6270 | CD | GLN | B | 169 | 22.321 | −96.272 | −32.275 | 1.00 | 48.75 | C |
| ATOM | 6271 | OE1 | GLN | B | 169 | 22.054 | −97.472 | −32.110 | 1.00 | 47.39 | O |
| ATOM | 6272 | NE2 | GLN | B | 169 | 22.751 | −95.480 | −31.284 | 1.00 | 42.54 | N |
| ATOM | 6273 | C | GLN | B | 169 | 23.056 | −96.149 | −36.697 | 1.00 | 44.90 | C |
| ATOM | 6274 | O | GLN | B | 169 | 21.906 | −96.631 | −36.919 | 1.00 | 41.94 | O |
| ATOM | 6225 | N | VAL | B | 170 | 24.136 | −96.137 | −37.206 | 1.00 | 45.70 | N |
| ATOM | 6276 | CA | VAL | B | 170 | 24.067 | −97.994 | −38.119 | 1.00 | 47.01 | C |
| ATOM | 6227 | CB | VAL | B | 170 | 25.281 | −98.054 | −39.068 | 1.00 | 46.19 | C |
| ATOM | 6278 | CG1 | VAL | B | 170 | 25.017 | −99.082 | −40.156 | 1.00 | 45.48 | C |
| ATOM | 6279 | CG2 | VAL | B | 170 | 25.600 | −96.679 | −39.667 | 1.00 | 42.20 | C |
| ATOM | 6280 | C | VAL | B | 170 | 23.992 | −99.314 | −37.357 | 1.00 | 44.86 | C |
| ATOM | 6281 | O | VAL | B | 170 | 24.732 | −99.526 | −36.405 | 1.00 | 45.97 | O |
| ATOM | 6282 | N | THR | B | 171 | 23.116 | −100.208 | −37.794 | 1.00 | 43.83 | N |
| ATOM | 6283 | CA | THR | B | 171 | 22.775 | −101.388 | −37.006 | 1.00 | 43.63 | C |
| ATOM | 6284 | CB | THR | B | 171 | 21.230 | −101.561 | −36.927 | 1.00 | 42.60 | C |
| ATOM | 6285 | DG1 | THR | B | 171 | 20.629 | −100.335 | −36.492 | 1.00 | 48.40 | O |
| ATOM | 6286 | CG2 | THR | B | 171 | 20.792 | −102.694 | −35.975 | 1.00 | 38.40 | C |
| ATOM | 6287 | C | THR | B | 171 | 23.460 | −102.635 | −37.571 | 1.00 | 48.79 | C |
| ATOM | 6288 | O | THR | B | 171 | 24.010 | −103.432 | −36.810 | 1.00 | 50.35 | O |
| ATOM | 6289 | N | ACYS | B | 172 | 23.406 | −102.817 | −38.897 | 0.50 | 49.19 | N |
| ATOM | 6290 | N | BCYS | B | 172 | 23.418 | −102.771 | −38.896 | 0.50 | 53.08 | N |
| ATOM | 6291 | CA | ACYS | B | 172 | 23.991 | −103.990 | −39.582 | 0.50 | 48.96 | C |
| ATOM | 6292 | CA | BCYS | B | 172 | 24.018 | −103.891 | −39.605 | 0.50 | 55.75 | C |
| ATOM | 6293 | CB | ACYS | B | 172 | 22.944 | −105.111 | −39.753 | 0.50 | 46.57 | C |
| ATOM | 6294 | CB | BCYS | B | 172 | 22.955 | −104.922 | −39.984 | 0.50 | 58.09 | C |
| ATOM | 6295 | SG | ACYS | B | 172 | 23.528 | −106.712 | −40.412 | 0.50 | 40.87 | S |
| ATOM | 6296 | SG | BCYS | B | 172 | 21.895 | −105.481 | −38.639 | 0.50 | 66.01 | S |
| ATOM | 6297 | C | ACYS | B | 172 | 24.524 | −103.600 | −40.952 | 0.50 | 50.55 | C |
| ATOM | 6298 | C | BCYS | B | 172 | 24.653 | −103.410 | −40.887 | 0.50 | 54.70 | C |
| ATOM | 6299 | O | ACYS | B | 172 | 23.863 | −102.860 | −41.689 | 0.50 | 49.76 | O |
| ATOM | 6300 | O | BCYS | B | 172 | 24.185 | −102.447 | −41.500 | 0.50 | 54.67 | O |
| ATOM | 6301 | N | VAL | B | 173 | 25.715 | −104.096 | −41.291 | 1.00 | 52.27 | N |
| ATOM | 6302 | CA | VAL | B | 173 | 26.202 | −104.032 | −42.678 | 1.00 | 48.77 | C |
| ATOM | 6303 | CB | VAL | B | 173 | 27.393 | −103.054 | −42.913 | 1.00 | 47.41 | C |
| ATOM | 6304 | CG1 | VAL | B | 173 | 20.952 | −101.616 | −42.735 | 1.00 | 43.70 | C |
| ATOM | 6305 | CG2 | VAL | B | 173 | 28.622 | −103.384 | −42.059 | 1.00 | 47.25 | C |
| ATOM | 6306 | C | VAL | B | 173 | 26.543 | −105.440 | −43.148 | 1.00 | 50.27 | C |
| ATOM | 6307 | O | VAL | B | 173 | 27.306 | −106.134 | −42.480 | 1.00 | 53.11 | O |
| ATOM | 6308 | N | ALA | B | 174 | 25.975 | −105.861 | −44.282 | 1.00 | 52.47 | N |
| ATOM | 6309 | CA | ALA | B | 174 | 26.292 | −107.177 | −44.881 | 1.00 | 53.02 | C |
| ATOM | 6310 | CB | ALA | B | 174 | 25.102 | −108.120 | −44.764 | 1.00 | 52.34 | C |
| ATOM | 6311 | C | ALA | B | 174 | 26.777 | −107.124 | −46.336 | 1.00 | 50.54 | C |
| ATOM | 6312 | O | ALA | B | 174 | 26.060 | −106.641 | −47.209 | 1.00 | 51.12 | O |
| ATOM | 6313 | N | ALA | B | 175 | 27.987 | −107.646 | −46.583 | 1.00 | 50.28 | N |
| ATOM | 6314 | CA | ALA | B | 175 | 28.581 | −107.745 | −47.941 | 1.00 | 45.14 | C |
| ATOM | 6315 | CB | ALA | B | 175 | 30.049 | −108.136 | −47.844 | 1.00 | 45.69 | C |
| ATOM | 6316 | C | ALA | B | 175 | 27.828 | −108.757 | −48.792 | 1.00 | 44.90 | C |
| ATOM | 6317 | O | ALA | B | 175 | 27.452 | −109.818 | −48.294 | 1.00 | 44.56 | O |
| ATOM | 6318 | N | SER | B | 176 | 27.583 | −108.446 | −50.067 | 1.00 | 47.56 | N |
| ATOM | 6319 | CA | SER | B | 176 | 26.955 | −109.452 | −50.960 | 1.00 | 47.57 | C |
| ATOM | 6320 | CB | SER | B | 176 | 26.675 | −108.501 | −52.359 | 1.00 | 45.92 | C |
| ATOM | 6321 | OG | SER | B | 176 | 27.878 | −108.659 | −53.045 | 1.00 | 50.75 | O |
| ATOM | 6322 | C | SER | B | 176 | 27.850 | −110.684 | −51.049 | 1.00 | 46.08 | C |
| ATOM | 6323 | O | SER | B | 176 | 29.064 | −110.568 | −50.870 | 1.00 | 44.08 | O |
| ATOM | 6324 | N | PRO | B | 177 | 27.253 | −111.868 | −51.294 | 1.00 | 49.69 | N |
| ATOM | 6325 | CA | PRO | B | 177 | 28.056 | −113.097 | −51.350 | 1.00 | 49.07 | C |
| ATOM | 6326 | CB | PRO | B | 177 | 27.035 | −114.182 | −51.712 | 1.00 | 47.99 | C |
| ATOM | 6327 | CG | PRO | B | 177 | 25.710 | −113.621 | −51.282 | 1.00 | 49.71 | C |
| ATOM | 6328 | CD | PRO | B | 177 | 25.817 | −112.137 | −51.523 | 1.00 | 46.58 | C |
| ATOM | 6329 | C | PRO | B | 177 | 29.156 | −113.030 | −52.411 | 1.00 | 53.54 | C |
| ATOM | 6330 | O | PRO | B | 177 | 30.275 | −113.488 | −52.142 | 1.00 | 53.83 | O |
| ATOM | 6331 | N | HIS | B | 178 | 28.866 | −112.423 | −53.570 | 1.00 | 52.06 | N |
| ATOM | 6332 | CA | HIS | B | 178 | 29.797 | −112.483 | −54.710 | 1.00 | 52.50 | C |
| ATOM | 6333 | CB | HIS | B | 178 | 29.238 | −113.416 | −55.798 | 1.00 | 50.65 | C |
| ATOM | 6334 | CG | HIS | B | 178 | 28.867 | −114.776 | −55.287 | 1.00 | 53.28 | C |
| ATOM | 6335 | ND1 | HIS | B | 178 | 29.785 | −115.626 | −54.692 | 1.00 | 52.11 | N |
| ATOM | 6336 | CE1 | HIS | B | 178 | 29.176 | −116.742 | −54.331 | 1.00 | 51.67 | C |
| ATOM | 6337 | NE2 | HIS | B | 178 | 27.900 | −116.652 | −54.673 | 1.00 | 54.90 | N |
| ATOM | 6338 | CD2 | HIS | B | 178 | 27.679 | −115.430 | −55.269 | 1.00 | 50.81 | C |
| ATOM | 6339 | C | HIS | B | 178 | 30.301 | −111.184 | −55.356 | 1.00 | 52.53 | C |
| ATOM | 6340 | O | HIS | B | 178 | 31.102 | −111.265 | −56.287 | 1.00 | 52.60 | O |
| ATOM | 6341 | N | LYS | B | 179 | 29.858 | −110.010 | −54.892 | 1.00 | 49.84 | N |
| ATOM | 6342 | CA | LYS | B | 179 | 30.275 | −108.735 | −55.522 | 1.00 | 48.91 | C |
| ATOM | 6343 | CB | LYS | B | 179 | 29.096 | −108.044 | −56.211 | 1.00 | 49.15 | C |
| ATOM | 6344 | CG | LYS | B | 179 | 28.364 | −108.894 | −57.232 | 1.00 | 51.10 | C |
| ATOM | 6345 | CD | LYS | B | 179 | 27.227 | −108.107 | −57.872 | 1.00 | 58.82 | C |
| ATOM | 6346 | CE | LYS | B | 179 | 26.029 | −108.994 | −58.184 | 1.00 | 59.99 | C |
| ATOM | 6347 | NZ | LYS | B | 179 | 25.014 | −108.272 | −58.997 | 1.00 | 65.72 | N |
| ATOM | 6348 | C | LYS | B | 179 | 30.926 | −107.773 | −54.546 | 1.00 | 49.84 | C |
| ATOM | 6349 | O | LYS | B | 179 | 30.287 | −107.313 | −53.589 | 1.00 | 49.87 | O |
| ATOM | 6350 | N | ASP | B | 180 | 32.195 | −107.464 | −54.796 | 1.00 | 54.50 | N |
| ATOM | 6351 | CA | ASP | B | 180 | 33.024 | −106.639 | −53.886 | 1.00 | 59.15 | C |
| ATOM | 6352 | CB | ASP | B | 180 | 34.449 | −106.479 | −54.448 | 1.00 | 66.77 | C |
| ATOM | 6353 | CG | ASP | B | 180 | 35.519 | −107.178 | −53.604 | 1.00 | 70.73 | C |
| ATOM | 6354 | OD1 | ASP | B | 180 | 35.238 | −108.223 | 52.076 | 1.00 | 66.36 | O |
| ATOM | 6355 | OD2 | ASP | B | 180 | 36.666 | −106.668 | −53.586 | 1.00 | 77.89 | O |
| ATOM | 6356 | C | ASP | B | 180 | 32.473 | −105.241 | −53.603 | 1.00 | 58.47 | C |
| ATOM | 6357 | O | ASP | B | 180 | 32.892 | −104.585 | −52.647 | 1.00 | 64.72 | O |
| ATOM | 6358 | N | SER | B | 181 | 31.549 | −104.785 | −54.442 | 1.00 | 53.04 | N |
| ATOM | 6359 | CA | SER | B | 181 | 31.110 | −103.407 | −54.412 | 1.00 | 48.52 | C |
| ATOM | 6360 | CB | SER | B | 181 | 31.335 | −102.786 | −55.787 | 1.00 | 49.90 | C |
| ATOM | 6361 | OG | SER | B | 181 | 30.420 | −103.340 | −56.715 | 1.00 | 53.21 | O |
| ATOM | 6362 | C | SER | B | 181 | 29.642 | −103.299 | −54.040 | 1.00 | 45.71 | C |
| ATOM | 6363 | O | SER | B | 181 | 29.067 | −102.220 | −54.101 | 1.00 | 46.22 | O |
| ATOM | 6364 | N | VAL | B | 182 | 29.037 | −104.426 | −53.689 | 1.00 | 43.92 | N |
| ATOM | 6365 | CA | VAL | B | 182 | 27.641 | −104.470 | −53.283 | 1.00 | 44.43 | C |
| ATOM | 6366 | CB | VAL | B | 182 | 26.792 | −105.316 | −54.257 | 1.00 | 44.94 | C |
| ATOM | 6367 | CG1 | VAL | B | 182 | 25.363 | −105.523 | −53.747 | 1.00 | 44.93 | C |
| ATOM | 6368 | CG2 | VAL | B | 182 | 26.765 | −104.663 | −55.626 | 1.00 | 46.16 | C |
| ATOM | 6369 | C | VAL | B | 182 | 27.520 | −105.006 | −51.862 | 1.00 | 46.65 | C |
| ATOM | 6370 | O | VAL | B | 182 | 28.048 | −106.073 | −51.540 | 1.00 | 49.07 | O |
| ATOM | 6371 | N | PHE | B | 183 | 26.829 | −104.244 | −51.017 | 1.00 | 50.34 | N |
| ATOM | 6372 | CA | PHE | B | 183 | 26.508 | −104.658 | −49.641 | 1.00 | 49.09 | C |
| ATOM | 6373 | CB | PHE | B | 183 | 27.672 | −104.353 | −48.698 | 1.00 | 46.11 | C |
| ATOM | 6374 | CG | PHE | B | 183 | 28.117 | −102.920 | −48.714 | 1.00 | 46.94 | C |
| ATOM | 6375 | CD1 | PHE | B | 183 | 27.456 | −101.958 | −47.948 | 1.00 | 48.29 | C |
| ATOM | 6376 | CE1 | PHE | B | 183 | 27.878 | −100.642 | −47.945 | 1.00 | 45.01 | C |
| ATOM | 6377 | CZ | PHE | B | 183 | 28.962 | −100.269 | −48.718 | 1.00 | 45.91 | C |
| ATOM | 6378 | CE2 | PHE | B | 183 | 29.633 | −101.216 | −49.474 | 1.00 | 47.13 | C |
| ATOM | 6379 | CD2 | PHE | B | 183 | 29.212 | −102.534 | −49.463 | 1.00 | 48.06 | C |
| ATOM | 6380 | C | PHE | B | 183 | 25.235 | −103.982 | −49.149 | 1.00 | 47.96 | C |
| ATOM | 6381 | O | PHE | B | 183 | 24.847 | −102.938 | −49.653 | 1.00 | 49.22 | O |
| ATOM | 6382 | N | LEU | B | 184 | 24.593 | −104.590 | −48.162 | 2.00 | 52.39 | N |
| ATOM | 6383 | CA | LEU | B | 184 | 23.383 | −104.042 | −47.558 | 1.00 | 49.46 | C |
| ATOM | 6384 | CB | LEU | B | 184 | 22.392 | −105.151 | −47.218 | 1.00 | 44.43 | C |
| ATOM | 6385 | CG | LEU | B | 184 | 21.700 | −105.373 | −48.367 | 1.00 | 46.28 | C |
| ATOM | 6386 | CD1 | LEU | B | 184 | 21.203 | −107.232 | −47.899 | 1.00 | 45.44 | C |
| ATOM | 6387 | CD2 | LEU | B | 184 | 20.567 | −105.063 | −48.989 | 1.00 | 44.51 | C |
| ATOM | 6388 | C | LEU | B | 184 | 23.760 | −103.337 | −46.279 | 1.00 | 50.58 | C |
| ATOM | 6389 | O | LEU | B | 184 | 24.724 | −103.121 | −45.624 | 1.00 | 50.61 | O |
| ATOM | 6390 | N | SER | B | 185 | 22.989 | −102.316 | −45.917 | 1.00 | 50.01 | N |
| ATOM | 6391 | CA | SER | B | 185 | 23.133 | −101.664 | −44.615 | 1.00 | 47.67 | C |
| ATOM | 6392 | CB | SER | B | 185 | 23.978 | −100.415 | −44.744 | 1.00 | 45.78 | C |
| ATOM | 6393 | OG | SER | B | 185 | 23.183 | −99.150 | −45.242 | 1.00 | 44.74 | O |
| ATOM | 6394 | C | SER | B | 185 | 21.748 | −101.303 | −44.080 | 1.00 | 50.24 | C |
| ATOM | 6395 | O | SER | B | 185 | 20.789 | −101.155 | −44.854 | 1.00 | 49.99 | O |
| ATOM | 6396 | N | CYS | B | 186 | 21.631 | −101.177 | −42.763 | 1.00 | 48.75 | N |
| ATOM | 6397 | CA | CYS | B | 186 | 20.363 | −100.803 | −42.165 | 1.00 | 47.23 | C |
| ATOM | 6398 | CB | CYS | B | 186 | 19.415 | −101.998 | −42.002 | 1.00 | 43.01 | C |
| ATOM | 6399 | SG | CYS | B | 186 | 19.837 | −103.169 | −40.696 | 1.00 | 47.04 | S |
| ATOM | 6400 | C | CYS | B | 186 | 20.565 | −100.044 | −40.861 | 1.00 | 49.34 | C |
| ATOM | 6401 | O | CYS | B | 186 | 21.466 | −100.346 | −40.077 | 1.00 | 50.86 | O |
| ATOM | 6402 | N | SER | B | 187 | 19.712 | −99.045 | −40.655 | 1.00 | 48.60 | N |
| ATOM | 6403 | CA | SER | B | 187 | 19.919 | −98.051 | −39.623 | 1.00 | 45.84 | C |
| ATOM | 6404 | CB | SER | B | 187 | 20.373 | −96.136 | −40.241 | 1.00 | 41.06 | C |
| ATOM | 6405 | OG | SER | B | 187 | 20.474 | −95.716 | −39.270 | 1.00 | 44.44 | O |
| ATOM | 6406 | C | SER | B | 187 | 18.652 | −97.135 | −38.829 | 1.00 | 46.18 | C |
| ATOM | 6407 | O | SER | B | 187 | 17.577 | −98.301 | −39.203 | 1.00 | 43.46 | O |
| ATOM | 6408 | N | GLU | B | 188 | 18.806 | −97.129 | −37.714 | 1.00 | 46.94 | N |
| ATOM | 6409 | CA | GLU | B | 188 | 17.680 | −96.136 | −36.892 | 1.00 | 47.52 | C |
| ATOM | 6410 | CB | GLU | B | 188 | 18.154 | −96.448 | −35.472 | 1.00 | 45.58 | C |
| ATOM | 6411 | CG | GLU | B | 188 | 18.032 | −97.687 | −34.604 | 1.00 | 50.71 | C |
| ATOM | 6412 | CD | GLU | B | 188 | 18.825 | −97.608 | −33.326 | 1.00 | 49.64 | C |
| ATOM | 6413 | OE1 | GLU | B | 188 | 19.251 | −96.505 | −32.942 | 1.00 | 50.80 | O |
| ATOM | 6414 | OE2 | GLU | B | 188 | 19.007 | −98.660 | −32.702 | 1.00 | 53.00 | O |
| ATOM | 6415 | C | GLU | B | 188 | 16.942 | −95.546 | −37.510 | 1.00 | 46.90 | C |
| ATOM | 6416 | O | GLU | B | 188 | 15.966 | −95.041 | −36.945 | 1.00 | 46.50 | O |
| ATOM | 6417 | N | ASP | B | 189 | 17.417 | −95.215 | −36.677 | 1.00 | 43.72 | N |
| ATOM | 6418 | CA | ASP | B | 189 | 16.789 | −94.028 | −39.392 | 1.00 | 44.30 | C |
| ATOM | 6419 | CB | ASP | B | 189 | 17.826 | −93.184 | −40.155 | 1.00 | 41.23 | C |
| ATOM | 6420 | CG | ASP | B | 189 | 18.343 | −93.158 | −41.406 | 1.00 | 40.78 | C |
| ATOM | 6421 | OD1 | ASP | B | 189 | 17.976 | −95.009 | −41.665 | 1.00 | 40.15 | O |
| ATOM | 6422 | OD2 | ASP | B | 189 | 19.132 | −93.221 | −42.145 | 1.00 | 44.73 | O |
| ATOM | 6423 | C | ASP | B | 189 | 15.694 | −94.195 | −40.289 | 1.00 | 42.28 | C |
| ATOM | 6424 | O | ASP | B | 189 | 15.146 | −93.904 | −41.132 | 1.00 | 40.60 | O |
| ATOM | 6425 | N | ASN | B | 190 | 15.405 | −95.174 | −40.079 | 1.00 | 41.95 | N |
| ATOM | 6426 | CA | ASN | B | 190 | 14.399 | −96.638 | −40.824 | 1.00 | 45.50 | C |
| ATOM | 6427 | CB | ASN | B | 190 | 13.009 | −95.981 | −40.788 | 1.00 | 43.91 | C |
| ATOM | 6428 | CG | ASN | B | 190 | 11.908 | −97.011 | −40.647 | 1.00 | 45.12 | C |
| ATOM | 6429 | OD1 | ASN | B | 190 | 12.181 | −98.200 | −40.605 | 1.00 | 46.40 | O |
| ATOM | 6430 | ND2 | ASN | B | 190 | 10.668 | −96.565 | −40.549 | 1.00 | 50.20 | N |
| ATOM | 6431 | C | ASN | B | 190 | 14.741 | −97.095 | −42.246 | 1.00 | 46.35 | C |
| ATOM | 6432 | O | ASN | B | 190 | 13.903 | −97.117 | −42.911 | 1.00 | 47.78 | O |
| ATOM | 6433 | N | ARG | B | 191 | 15.962 | −96.810 | −47.698 | 1.00 | 45.43 | N |
| ATOM | 6434 | CA | ARG | B | 191 | 16.380 | −97.170 | −44.053 | 1.00 | 45.94 | C |
| ATOM | 6435 | CB | ARG | B | 191 | 17.267 | −96.086 | −44.652 | 1.00 | 43.60 | C |
| ATOM | 6436 | CG | ARG | B | 191 | 16.589 | −94.143 | −44.898 | 1.00 | 42.05 | C |
| ATOM | 6437 | CD | ARG | B | 191 | 17.689 | −93.167 | −45.235 | 1.00 | 43.46 | C |
| ATOM | 6438 | NE | ARG | B | 191 | 17.225 | −92.499 | −45.771 | 1.00 | 43.63 | N |
| ATOM | 6439 | CZ | ARG | B | 191 | 17.211 | −91.359 | −45.095 | 1.00 | 42.08 | C |
| ATOM | 6440 | NH1 | ARG | B | 191 | 17.622 | −91.313 | −43.835 | 1.00 | 38.04 | N |
| ATOM | 6441 | NH2 | ARG | B | 191 | 16.783 | −90.259 | −45.693 | 1.00 | 50.38 | N |
| ATOM | 6442 | C | ARG | B | 191 | 17.122 | −98.495 | −44.124 | 1.00 | 48.00 | C |
| ATOM | 6443 | O | ARG | B | 191 | 18.018 | −98.765 | −43.327 | 1.00 | 53.86 | O |
| ATOM | 6444 | N | ILE | B | 192 | 16.724 | −99.329 | −45.073 | 1.00 | 49.37 | N |
| ATOM | 6445 | CA | ILE | B | 192 | 17.628 | −100.332 | −45.615 | 1.00 | 46.91 | C |
| ATOM | 6446 | CB | ILE | B | 192 | 16.957 | −101.685 | −45.850 | 1.00 | 46.17 | C |
| ATOM | 6447 | CG1 | ILE | B | 192 | 16.268 | −102.172 | −44.576 | 1.00 | 42.96 | C |
| ATOM | 6448 | CD1 | ILE | B | 192 | 15.325 | −103.330 | −44.795 | 1.00 | 46.49 | C |
| ATOM | 6449 | CG2 | ILE | B | 192 | 18.017 | −102.681 | −46.291 | 1.00 | 48.69 | C |
| ATOM | 6450 | C | ILE | B | 192 | 18.205 | −99.823 | −46.935 | 1.00 | 46.90 | C |
| ATOM | 6451 | O | ILE | B | 192 | 17.469 | −99.528 | −47.874 | 1.00 | 49.05 | O |
| ATOM | 6452 | N | LEU | B | 193 | 19.527 | −99.723 | −46.995 | 1.00 | 48.29 | N |
| ATOM | 6453 | CA | LEU | B | 193 | 20.209 | −99.310 | −48.209 | 1.00 | 48.54 | C |
| ATOM | 6454 | CB | LEU | B | 193 | 21.191 | −98.192 | −47.903 | 1.00 | 45.96 | C |
| ATOM | 6455 | CG | LEU | B | 193 | 20.613 | −96.909 | −47.334 | 1.00 | 45.52 | C |
| ATOM | 6456 | CD1 | LEU | B | 193 | 21.733 | −95.956 | −46.961 | 1.00 | 42.85 | C |
| ATOM | 6457 | CD2 | LEU | B | 193 | 19.670 | −96.273 | −48.341 | 1.00 | 48.40 | C |
| ATOM | 6458 | C | LEU | B | 193 | 20.948 | −100.445 | −48.923 | 1.00 | 52.72 | C |
| ATOM | 6459 | O | LEU | B | 193 | 21.848 | −101.087 | −48.356 | 1.00 | 52.06 | O |
| ATOM | 6460 | N | LEU | B | 194 | 20.552 | −100.676 | −50.176 | 1.00 | 55.02 | N |
| ATOM | 6461 | CA | LEU | B | 194 | 21.345 | −101.448 | −51.124 | 1.00 | 51.05 | C |
| ATOM | 6462 | CB | LEU | B | 194 | 20.458 | −101.972 | −52.256 | 1.00 | 52.26 | C |
| ATOM | 6463 | CG | LEU | B | 194 | 21.049 | −102.947 | −53.293 | 1.00 | 49.95 | C |
| ATOM | 6464 | CD1 | LEU | B | 194 | 21.608 | −104.180 | −52.601 | 1.00 | 44.60 | C |
| ATOM | 6465 | CD2 | LEU | B | 194 | 19.996 | −103.344 | −54.323 | 1.00 | 48.50 | C |
| ATOM | 6466 | C | LEU | B | 194 | 22.471 | −100.597 | −51.698 | 1.00 | 47.06 | C |
| ATOM | 6467 | O | LEU | B | 194 | 22.225 | −99.581 | −52.349 | 1.00 | 46.72 | O |
| ATOM | 6468 | N | TRP | B | 195 | 23.708 | −101.005 | −51.461 | 1.00 | 46.76 | N |
| ATOM | 6469 | CA | TRP | B | 195 | 24.864 | −100.256 | −51.940 | 1.00 | 45.60 | C |
| ATOM | 6470 | CB | TRP | B | 195 | 25.923 | −100.198 | −50.869 | 1.00 | 48.32 | C |
| ATOM | 6471 | CG | TRP | B | 195 | 25.516 | −99.421 | −49.681 | 1.00 | 51.22 | C |
| ATOM | 6472 | CD1 | TRP | B | 195 | 24.689 | −99.831 | −48.668 | 1.00 | 49.99 | C |
| ATOM | 6473 | NE1 | TRP | B | 195 | 24.564 | −98.839 | −47.733 | 1.00 | 49.37 | N |
| ATOM | 6474 | CE2 | TRP | B | 195 | 25.325 | −97.767 | −48.115 | 1.00 | 48.23 | C |
| ATOM | 6475 | CD2 | TRP | B | 195 | 25.939 | −98.101 | −49.346 | 1.00 | 49.54 | C |
| ATOM | 6476 | CE3 | TRP | B | 195 | 26.783 | −97.164 | −49.964 | 1.00 | 50.45 | C |
| ATOM | 6477 | CZ3 | TRP | B | 195 | 26.973 | −95.928 | −49.343 | 1.00 | 52.27 | C |
| ATOM | 6478 | CH2 | TRP | B | 195 | 26.331 | −95.624 | −48.119 | 1.00 | 50.79 | C |
| ATOM | 6479 | CZ2 | TRP | B | 195 | 25.513 | −96.535 | −47.492 | 1.00 | 48.64 | C |
| ATOM | 6480 | C | TRP | B | 195 | 25.474 | −100.677 | −55.160 | 1.00 | 46.67 | C |
| ATOM | 6481 | O | TRP | B | 195 | 25.385 | −102.082 | −53.378 | 1.00 | 49.56 | O |
| ATOM | 6482 | N | ASP | B | 196 | 26.051 | −100.027 | −54.022 | 1.00 | 46.95 | N |
| ATOM | 6483 | CA | ASP | B | 196 | 26.861 | −100.427 | −55.153 | 1.00 | 45.30 | C |
| ATOM | 6484 | CB | ASP | B | 196 | 26.036 | −100.677 | −56.421 | 1.00 | 45.57 | C |
| ATOM | 6485 | CG | ASP | B | 196 | 26.857 | −101.404 | −57.527 | 1.00 | 48.42 | C |
| ATOM | 6486 | OD1 | ASP | B | 196 | 28.128 | −101.365 | −57.515 | 1.00 | 48.16 | O |
| ATOM | 6487 | OD2 | ASP | B | 196 | 26.225 | −102.025 | −58.407 | 1.00 | 42.72 | O |
| ATOM | 6488 | C | ASP | B | 196 | 27.826 | −99.306 | −55.421 | 1.00 | 43.95 | C |
| ATOM | 6489 | O | ASP | B | 196 | 27.464 | −98.326 | −56.074 | 1.00 | 40.58 | O |
| ATOM | 6490 | N | THR | B | 197 | 29.056 | −99.456 | −54.940 | 1.00 | 43.27 | N |
| ATOM | 6491 | CA | THR | B | 197 | 30.043 | −98.386 | −55.048 | 1.00 | 45.83 | C |
| ATOM | 6492 | CB | THR | B | 197 | 31.271 | −98.628 | −54.145 | 1.00 | 47.18 | C |
| ATOM | 6493 | OG1 | THR | B | 197 | 31.855 | −99.902 | −54.436 | 1.00 | 47.32 | O |
| ATOM | 6494 | CG2 | THR | B | 197 | 30.866 | −98.569 | −52.681 | 1.00 | 50.63 | C |
| ATOM | 6495 | C | THR | B | 197 | 30.504 | −98.061 | −56.475 | 1.00 | 46.51 | C |
| ATOM | 6496 | O | THR | B | 197 | 31.317 | −97.149 | −56.677 | 1.00 | 47.70 | O |
| ATOM | 6497 | N | ARG | B | 198 | 29.989 | −98.790 | −57.459 | 1.00 | 45.50 | N |
| ATOM | 6498 | CA | ARG | B | 198 | 30.303 | −98.487 | −58.856 | 1.00 | 51.52 | C |
| ATOM | 6499 | CB | ARG | B | 198 | 30.040 | −99.705 | −59.754 | 1.00 | 52.25 | C |
| ATOM | 6500 | CG | ARG | B | 198 | 31.047 | −100.852 | −59.565 | 1.00 | 45.62 | C |
| ATOM | 6501 | CD | ARG | B | 198 | 30.623 | −102.140 | −60.277 | 1.00 | 44.90 | C |
| ATOM | 6502 | NE | ARG | B | 198 | 29.288 | −102.600 | −59.897 | 1.00 | 45.05 | N |
| ATOM | 6503 | CZ | ARG | B | 198 | 28.791 | −103.816 | −60.126 | 1.00 | 47.24 | C |
| ATOM | 6504 | NB1 | ARG | B | 198 | 29.506 | −104.744 | −60.747 | 1.00 | 52.04 | N |
| ATOM | 6505 | NB2 | ARG | B | 198 | 27.565 | −104.115 | −59.723 | 1.00 | 41.66 | 5 |
| ATOM | 6506 | C | ARG | B | 198 | 29.540 | −97.242 | −59.336 | 1.00 | 52.90 | C |
| ATOM | 6507 | O | ARG | B | 198 | 30.025 | −96.500 | −60.198 | 1.00 | 54.00 | O |
| ATOM | 6508 | N | CYS | B | 199 | 28.364 | −97.024 | −58.738 | 1.00 | 50.58 | N |
| ATOM | 6509 | CA | CYS | B | 199 | 27.496 | −95.867 | −58.976 | 1.00 | 49.57 | C |
| ATOM | 6510 | CB | CYS | B | 199 | 26.117 | −96.167 | −58.426 | 1.00 | 45.40 | C |
| ATOM | 6511 | SG | CYS | B | 199 | 25.407 | −97.643 | −59.169 | 1.00 | 47.17 | S |
| ATOM | 6512 | O | CYS | B | 199 | 27.980 | −94.547 | −58.367 | 1.00 | 52.36 | C |
| ATOM | 6513 | O | CYS | B | 199 | 28.766 | −94.529 | −57.251 | 1.00 | 50.67 | O |
| ATOM | 6514 | N | PRO | B | 200 | 27.766 | −93.427 | −59.089 | 1.00 | 54.08 | N |
| ATOM | 6515 | CA | PRO | B | 200 | 28.125 | −92.109 | −58.561 | 1.00 | 58.63 | C |
| ATOM | 6516 | CB | PRO | B | 200 | 27.608 | −91.149 | −59.640 | 1.00 | 59.65 | C |
| ATOM | 6517 | CG | PRO | B | 200 | 27.678 | −91.952 | −60.896 | 1.00 | 57.61 | C |
| ATOM | 6518 | CD | PRO | B | 200 | 27.223 | −93.324 | −60.455 | 1.00 | 56.34 | C |
| ATOM | 6519 | C | PRO | B | 200 | 27.432 | −91.150 | −57.223 | 1.00 | 59.01 | C |
| ATOM | 6520 | O | PRO | B | 200 | 28.075 | −91.182 | −56.286 | 1.00 | 58.67 | O |
| ATOM | 6521 | N | LYS | B | 201 | 26.138 | −92.173 | −57.148 | 1.00 | 56.98 | N |
| ATOM | 6522 | CA | LYS | B | 201 | 25.402 | −92.192 | −55.893 | 1.00 | 51.10 | C |
| ATOM | 6523 | CB | LYS | B | 201 | 24.077 | −91.430 | −56.024 | 1.00 | 55.44 | C |
| ATOM | 6524 | CG | LYS | B | 201 | 24.220 | −89.914 | −56.002 | 1.00 | 58.80 | C |
| ATOM | 6525 | CD | LYS | B | 201 | 22.860 | −89.147 | −56.191 | 1.00 | 61.49 | C |
| ATOM | 6526 | CE | LYS | B | 201 | 22.898 | −87.159 | −55.859 | 1.00 | 62.02 | C |
| ATOM | 6527 | NZ | LYS | B | 201 | 21.531 | −87.198 | −55.609 | 1.00 | 63.13 | N |
| ATOM | 6528 | C | LYS | B | 201 | 25.158 | −93.650 | −55.488 | 1.00 | 49.48 | C |
| ATOM | 6529 | O | LYS | B | 201 | 24.182 | −94.263 | −55.944 | 1.00 | 48.49 | O |
| ATOM | 6530 | N | PRO | B | 202 | 26.045 | −94.199 | −54.621 | 1.00 | 47.46 | N |
| ATOM | 6531 | CA | PRO | B | 202 | 26.190 | −95.614 | −54.241 | 1.00 | 45.00 | C |
| ATOM | 6532 | CB | PRO | B | 202 | 27.373 | −95.601 | −53.279 | 1.00 | 46.03 | C |
| ATOM | 6533 | CG | PRO | B | 202 | 27.649 | −94.175 | −52.948 | 1.00 | 42.61 | C |
| ATOM | 6534 | CD | PRO | B | 202 | 27.177 | −93.397 | −54.123 | 1.00 | 44.35 | C |
| ATOM | 6535 | C | PRO | B | 202 | 25.018 | −96.298 | −53.557 | 1.00 | 49.69 | C |
| ATOM | 6536 | O | PRO | B | 202 | 24.756 | −97.483 | −53.813 | 1.00 | 54.45 | O |
| ATOM | 6537 | N | ALA | B | 203 | 24.330 | −95.589 | −52.673 | 1.00 | 49.65 | N |
| ATOM | 6538 | CA | ALA | B | 203 | 23.228 | −96.204 | −51.929 | 1.00 | 49.10 | C |
| ATOM | 6539 | CB | ALA | B | 203 | 23.117 | −95.587 | −50.545 | 1.00 | 49.64 | C |
| ATOM | 6540 | C | ALA | B | 203 | 21.912 | −96.061 | −52.666 | 1.00 | 48.26 | C |
| ATOM | 6541 | O | ALA | B | 203 | 21.723 | −95.146 | −53.465 | 1.00 | 52.14 | O |
| ATOM | 6542 | N | SER | B | 204 | 21.000 | −96.972 | −52.393 | 1.00 | 47.53 | N |
| ATOM | 6543 | CA | SER | B | 204 | 19.659 | −96.885 | −52.922 | 1.00 | 52.70 | C |
| ATOM | 6544 | CB | SER | B | 204 | 19.546 | −97.635 | −54.243 | 1.00 | 54.21 | C |
| ATOM | 6545 | OG | SER | B | 204 | 18.191 | −97.950 | −54.522 | 1.00 | 58.59 | O |
| ATOM | 6546 | C | SER | B | 204 | 18.731 | −97.484 | −51.876 | 1.00 | 56.19 | C |
| ATOM | 6547 | O | SER | B | 204 | 18.994 | −98.578 | −51.363 | 1.00 | 51.72 | O |
| ATOM | 6548 | N | GLN | B | 205 | 17.662 | −96.154 | −51.557 | 1.00 | 56.91 | N |
| ATOM | 6549 | CA | GLN | B | 205 | 16.752 | −97.140 | −50.479 | 1.00 | 59.16 | C |
| ATOM | 6550 | CB | GLN | B | 205 | 15.994 | −95.916 | −49.938 | 1.00 | 59.60 | C |
| ATOM | 6551 | CG | GLN | B | 205 | 14.966 | −96.256 | −48.868 | 1.00 | 64.73 | C |
| ATOM | 6552 | CD | GLN | B | 205 | 14.597 | −95.082 | −47.978 | 1.00 | 66.53 | C |
| ATOM | 6553 | OE1 | GLN | B | 205 | 15.097 | −93.959 | −48.139 | 1.00 | 61.28 | O |
| ATOM | 6554 | NE2 | GLN | B | 205 | 13.720 | −95.347 | −47.014 | 1.00 | 68.46 | N |
| ATOM | 6555 | C | GLN | B | 205 | 15.787 | −98.227 | −50.920 | 1.00 | 57.60 | C |
| ATOM | 6556 | O | GLN | B | 205 | 15.091 | −98.077 | −51.922 | 1.00 | 58.47 | O |
| ATOM | 6557 | N | ILE | B | 206 | 15.752 | −99.322 | −50.171 | 1.00 | 58.48 | N |
| ATOM | 6558 | CA | ILE | B | 206 | 14.923 | −100.466 | −50.545 | 1.00 | 63.78 | C |
| ATOM | 6559 | CB | ILE | B | 206 | 15.765 | −101.593 | −51.226 | 1.00 | 67.07 | C |
| ATOM | 6560 | CG1 | ILE | B | 206 | 14.872 | −102.773 | −51.641 | 1.00 | 73.77 | C |
| ATOM | 6561 | CD1 | ILE | B | 206 | 15.280 | −103.473 | −52.926 | 1.00 | 81.52 | C |
| ATOM | 6562 | CG2 | ILE | B | 206 | 16.932 | −102.048 | −50.347 | 1.00 | 59.50 | C |
| ATOM | 6563 | C | ILE | B | 206 | 14.094 | −100.982 | −49.367 | 1.00 | 65.05 | C |
| ATOM | 6564 | O | ILE | B | 206 | 14.389 | −100.677 | −48.216 | 1.00 | 71.43 | O |
| ATOM | 6565 | N | GLY | B | 207 | 13.058 | −101.755 | −49.668 | 1.00 | 69.02 | N |
| ATOM | 6566 | CA | GLY | B | 207 | 12.215 | −102.363 | −48.648 | 1.00 | 76.76 | C |
| ATOM | 6567 | C | GLY | B | 207 | 10.752 | −102.374 | −49.050 | 1.00 | 83.48 | C |
| ATOM | 6568 | O | GLY | B | 207 | 10.408 | −102.035 | −50.186 | 1.00 | 84.83 | O |
| ATOM | 6569 | N | CYS | B | 208 | 9.890 | −102.747 | −48.107 | 1.00 | 87.58 | N |
| ATOM | 6570 | CA | CYS | B | 208 | 8.464 | −102.931 | −48.384 | 1.00 | 97.06 | C |
| ATOM | 6571 | CB | CYS | B | 208 | 7.750 | −103.532 | −47.168 | 1.00 | 96.17 | C |
| ATOM | 6572 | SG | CYS | B | 208 | 8.613 | −104.508 | −46.361 | 1.00 | 97.87 | S |
| ATOM | 6573 | C | CYS | B | 208 | 7.779 | −101.637 | −48.826 | 1.00 | 100.95 | C |
| ATOM | 6574 | O | CYS | B | 208 | 8.355 | −100.547 | −48.709 | 1.00 | 97.23 | O |
| ATOM | 6575 | N | SER | B | 209 | 6.559 | −101.775 | −49.350 | 1.00 | 103.64 | N |
| ATOM | 6576 | CA | SER | B | 209 | 5.740 | −100.627 | −49.742 | 1.00 | 101.10 | C |
| ATOM | 6577 | CB | SER | B | 209 | 4.325 | −101.072 | −50.132 | 1.00 | 97.87 | C |
| ATOM | 6578 | OG | SER | B | 209 | 4.353 | −102.009 | −51.190 | 1.00 | 50.13 | O |
| ATOM | 6579 | C | SER | B | 209 | 5.682 | −99.610 | −48.600 | 1.00 | 98.95 | C |
| ATOM | 6580 | O | SER | B | 209 | 6.337 | −98.566 | −48.661 | 1.00 | 90.82 | O |
| ATOM | 6581 | N | ALA | B | 210 | 4.917 | −99.939 | −47.557 | 1.00 | 97.90 | N |
| ATOM | 6582 | CA | ALA | B | 210 | 4.796 | −99.087 | −46.374 | 1.00 | 94.18 | C |
| ATOM | 6583 | CB | ALA | B | 210 | 3.538 | −99.437 | −45.585 | 1.00 | 88.51 | C |
| ATOM | 6584 | C | ALA | B | 210 | 6.033 | −99.204 | −45.484 | 1.00 | 89.21 | C |
| ATOM | 6585 | O | ALA | B | 210 | 6.482 | −100.328 | −45.197 | 1.00 | 80.08 | O |
| ATOM | 6586 | N | PRO | B | 211 | 6.606 | −98.046 | −45.073 | 1.00 | 85.86 | N |
| ATOM | 6587 | CA | PRO | B | 211 | 7.574 | −98.033 | −43.972 | 1.00 | 81.23 | C |
| ATOM | 6588 | CB | PRO | B | 211 | 7.792 | −96.539 | −43.717 | 1.00 | 78.50 | C |
| ATOM | 6589 | CG | PRO | B | 211 | 7.619 | −95.928 | −45.066 | 1.00 | 80.90 | C |
| ATOM | 6590 | CD | PRO | B | 211 | 6.526 | −96.727 | −45.735 | 1.00 | 82.92 | C |
| ATOM | 6591 | C | PRO | B | 211 | 6.989 | −98.729 | −42.738 | 1.00 | 72.06 | C |
| ATOM | 6592 | O | PRO | B | 211 | 5.810 | −98.540 | −42.404 | 1.00 | 67.44 | O |
| ATOM | 6593 | N | GLY | B | 212 | 7.807 | −99.562 | −42.106 | 1.00 | 61.06 | N |
| ATOM | 6594 | CA | GLY | B | 212 | 7.378 | −100.350 | −40.970 | 1.00 | 58.55 | C |
| ATOM | 6595 | C | GLY | B | 212 | 8.255 | −100.148 | −39.747 | 1.00 | 56.60 | C |
| ATOM | 6596 | O | GLY | B | 212 | 8.695 | −99.029 | −39.436 | 1.00 | 55.60 | O |
| ATOM | 6597 | N | TYR | B | 213 | 8.509 | −101.247 | −39.051 | 1.00 | 51.17 | N |
| ATOM | 6598 | CA | TYR | B | 213 | 9.328 | −101.218 | −37.860 | 1.00 | 48.44 | C |
| ATOM | 6599 | CB | TYR | B | 213 | 8.962 | −102.409 | −36.999 | 1.00 | 48.19 | C |
| ATOM | 6600 | CG | TYR | B | 213 | 7.582 | −102.276 | −36.392 | 1.00 | 50.77 | C |
| ATOM | 6601 | CD1 | TYR | B | 213 | 7.314 | −101.287 | −35.432 | 1.00 | 52.96 | C |
| ATOM | 6602 | CE1 | TYR | B | 213 | 6.061 | −101.152 | −34.856 | 1.00 | 50.08 | C |
| ATOM | 6603 | CZ | TYR | B | 213 | 5.047 | −102.013 | −35.239 | 1.00 | 53.49 | C |
| ATOM | 6604 | OH | TYR | B | 213 | 3.809 | −101.876 | −34.666 | 1.00 | 43.47 | O |
| ATOM | 6605 | CE2 | TYR | B | 213 | 5.278 | −103.006 | −36.191 | 1.00 | 53.93 | C |
| ATOM | 6606 | CD2 | TYR | B | 213 | 0.542 | −105.150 | −36.765 | 1.00 | 52.74 | C |
| ATOM | 6607 | C | TYR | B | 213 | 10.805 | −101.203 | −38.242 | 1.00 | 47.05 | C |
| ATOM | 6608 | O | TYR | B | 213 | 11.170 | −101.694 | −39.309 | 1.00 | 47.43 | O |
| ATOM | 6609 | N | LEU | B | 214 | 11.645 | −100.611 | −37.392 | 1.00 | 48.80 | N |
| ATOM | 6610 | CA | LEU | B | 214 | 13.066 | −100.400 | −37.721 | 1.00 | 44.85 | C |
| ATOM | 6611 | CB | LEU | B | 214 | 13.820 | −99.742 | −36.566 | 1.00 | 43.48 | C |
| ATOM | 6612 | CG | LEU | B | 214 | 13.448 | −98.318 | −36.156 | 1.00 | 47.66 | C |
| ATOM | 6613 | CD1 | LEU | B | 214 | 14.347 | −97.815 | −35.025 | 1.00 | 43.61 | C |
| ATOM | 6614 | CD2 | LEU | B | 214 | 13.501 | −97.372 | −37.351 | 1.00 | 45.37 | C |
| ATOM | 6615 | C | LEU | B | 214 | 13.751 | −101.711 | −38.080 | 1.00 | 45.96 | C |
| ATOM | 6616 | O | LEU | B | 214 | 13.457 | −102.744 | −37.471 | 1.00 | 47.77 | O |
| ATOM | 6617 | N | PRO | B | 215 | 14.673 | −101.673 | −39.062 | 1.00 | 43.74 | N |
| ATOM | 6618 | CA | PRO | B | 215 | 15.379 | −102.881 | −39.460 | 1.00 | 40.33 | C |
| ATOM | 6619 | CB | PRO | B | 215 | 15.869 | −102.542 | −40.878 | 1.00 | 30.81 | C |
| ATOM | 6620 | CG | PRO | B | 215 | 15.931 | −101.047 | −40.943 | 1.00 | 35.55 | C |
| ATOM | 6621 | CD | PRO | B | 215 | 15.163 | −100.482 | −39.787 | 1.00 | 41.25 | C |
| ATOM | 6622 | C | PRO | B | 215 | 16.530 | −103.152 | −38.519 | 1.00 | 33.29 | C |
| ATOM | 6623 | O | PRO | B | 215 | 17.248 | −102.221 | −38.169 | 1.00 | 41.02 | O |
| ATOM | 6624 | N | THR | B | 216 | 16.714 | −104.409 | −38.122 | 1.00 | 33.06 | N |
| ATOM | 6625 | CA | THR | B | 216 | 17.705 | −104.754 | −37.087 | 1.00 | 45.97 | C |
| ATOM | 6626 | CB | THR | B | 216 | 17.030 | −105.169 | −35.758 | 1.00 | 41.44 | C |
| ATOM | 6627 | OG1 | THR | B | 216 | 16.092 | −106.248 | −35.984 | 1.00 | 40.41 | O |
| ATOM | 6628 | CG2 | THR | B | 216 | 16.323 | −103.971 | −35.124 | 1.00 | 44.92 | C |
| ATOM | 6629 | C | THR | B | 216 | 18.690 | −105.864 | −37.455 | 1.00 | 47.42 | C |
| ATOM | 6630 | O | THR | B | 216 | 19.640 | −106.099 | −36.718 | 1.00 | 51.78 | O |
| ATOM | 6631 | N | SER | B | 217 | 18.462 | −106.566 | −38.565 | 1.00 | 48.48 | N |
| ATOM | 6632 | CA | SER | B | 217 | 19.376 | −107.632 | −38.990 | 1.00 | 49.17 | C |
| ATOM | 6633 | CB | SER | B | 217 | 19.180 | −108.887 | −38.143 | 1.00 | 50.29 | C |
| ATOM | 6634 | OG | SER | B | 217 | 17.808 | −109.268 | −38.108 | 1.00 | 52.33 | O |
| ATOM | 6635 | C | SER | B | 217 | 19.197 | −107.969 | −40.468 | 1.00 | 51.83 | C |
| ATOM | 6636 | O | SER | B | 217 | 18.069 | −107.916 | −40.994 | 1.00 | 43.46 | O |
| ATOM | 6637 | N | LEU | B | 218 | 20.311 | −108.313 | −41.127 | 1.00 | 47.73 | N |
| ATOM | 6638 | CA | LEU | B | 218 | 20.305 | −108.648 | −42.553 | 1.00 | 46.63 | C |
| ATOM | 6639 | CB | LEU | B | 218 | 20.844 | −107.485 | −43.390 | 1.00 | 41.60 | C |
| ATOM | 6640 | CG | LEU | B | 218 | 20.485 | −106.032 | −43.130 | 1.00 | 41.90 | C |
| ATOM | 6641 | CD1 | LEU | B | 218 | 21.682 | −105.131 | −43.439 | 1.00 | 41.08 | C |
| ATOM | 6642 | CD2 | LEU | B | 218 | 19.246 | −105.624 | −43.928 | 1.00 | 43.61 | C |
| ATOM | 6643 | C | LEU | B | 218 | 21.148 | −109.871 | −42.892 | 100 | 50.36 | C |
| ATOM | 6644 | O | LEU | B | 218 | 22.262 | −110.039 | −42.373 | 100 | 53.19 | O |
| ATOM | 6645 | N | ALA | B | 219 | 20.639 | −110.694 | −43.804 | 1.00 | 50.75 | N |
| ATOM | 6646 | CA | ALA | B | 219 | 21.427 | −111.181 | −44.382 | 1.00 | 51.21 | C |
| ATOM | 6647 | CB | ALA | B | 219 | 21.214 | −113.066 | −43.602 | 1.00 | 52.00 | C |
| ATOM | 6648 | C | ALA | B | 219 | 21.074 | −111.993 | −45.850 | 1.00 | 53.82 | C |
| ATOM | 6649 | O | ALA | B | 219 | 19.902 | −112.179 | −46.198 | 1.00 | 52.45 | O |
| ATOM | 6650 | N | TRP | B | 220 | 22.091 | −111.968 | −46.711 | 1.00 | 55.86 | N |
| ATOM | 6651 | CA | TRP | B | 220 | 21.908 | −112.318 | −48.119 | 1.00 | 54.14 | C |
| ATOM | 6652 | CB | TRP | B | 220 | 23.176 | −112.018 | −48.907 | 1.00 | 51.59 | C |
| ATOM | 6653 | CG | TRP | B | 220 | 23.490 | −110.568 | −49.136 | 1.00 | 49.01 | C |
| ATOM | 6654 | CD1 | TRP | B | 220 | 24.320 | −109.776 | −48.385 | 1.00 | 51.13 | C |
| ATOM | 6655 | NE1 | TRP | B | 220 | 24.392 | −108.506 | −48.929 | 1.00 | 48.54 | N |
| ATOM | 6656 | CE2 | TRP | B | 220 | 23.613 | −108.468 | −50.057 | 1.00 | 46.59 | C |
| ATOM | 6657 | CD2 | TRP | B | 220 | 23.037 | −109.152 | −50.224 | 1.00 | 47.66 | C |
| ATOM | 6658 | CE3 | TRP | B | 220 | 22.187 | −109.975 | −51.317 | 1.00 | 52.10 | C |
| ATOM | 6659 | CZ3 | TRP | B | 220 | 21.941 | −108.915 | −52.203 | 1.00 | 48.19 | C |
| ATOM | 6660 | CH2 | TRP | B | 220 | 22.536 | −107.656 | −52.005 | 1.00 | 47.61 | C |
| ATOM | 6661 | CZ2 | TRP | B | 220 | 23.366 | −107.415 | −50.941 | 1.00 | 45.73 | C |
| ATOM | 6662 | C | TRP | B | 220 | 21.627 | −113.807 | −48.215 | 1.00 | 52.77 | C |
| ATOM | 6663 | O | TRP | B | 220 | 22.076 | −114.564 | −47.362 | 1.00 | 53.16 | O |
| ATOM | 6664 | N | HIS | B | 221 | 20.883 | −114.223 | −49.238 | 1.00 | 58.39 | N |
| ATOM | 6665 | CA | HIS | B | 221 | 20.708 | −115.654 | −49.546 | 1.00 | 63.78 | C |
| ATOM | 6666 | CB | HIS | B | 221 | 19.549 | −115.864 | −50.511 | 1.00 | 68.61 | C |
| ATOM | 6667 | CG | HIS | B | 221 | 19.009 | −117.260 | −50.528 | 1.00 | 75.84 | C |
| ATOM | 6668 | ND1 | HIS | B | 221 | 19.528 | −118.250 | −51.335 | 1.00 | 83.58 | N |
| ATOM | 6669 | CE1 | HIS | B | 221 | 18.844 | −119.367 | −51.154 | 1.00 | 83.69 | C |
| ATOM | 6670 | NE2 | HIS | B | 221 | 17.891 | −119.133 | −50.269 | 1.00 | 76.83 | N |
| ATOM | 6671 | CD2 | HIS | B | 221 | 17.971 | −117.823 | −49.864 | 1.00 | 75.45 | C |
| ATOM | 6672 | C | HIS | B | 221 | 21.988 | −116.132 | −50.200 | 1.00 | 62.91 | C |
| ATOM | 6673 | O | HIS | B | 221 | 22.505 | −115.460 | −51.093 | 1.00 | 62.25 | O |
| ATOM | 6674 | N | PRO | B | 222 | 22.513 | −117.289 | −49.765 | 1.00 | 63.45 | N |
| ATOM | 6675 | CA | PRO | B | 222 | 23.843 | −117.624 | −50.260 | 1.00 | 63.40 | C |
| ATOM | 6676 | CB | PRO | B | 222 | 24.312 | −118.738 | −49.319 | 1.00 | 60.35 | C |
| ATOM | 6677 | CG | PRO | B | 222 | 23.121 | −119.144 | −48.506 | 1.00 | 61.84 | C |
| ATOM | 6678 | CD | PRO | B | 222 | 21.922 | −118.177 | −48.969 | 1.00 | 61.06 | C |
| ATOM | 6679 | C | PRO | B | 222 | 23.881 | −118.061 | −51.741 | 1.00 | 67.15 | C |
| ATOM | 6680 | O | PRO | B | 222 | 24.963 | −118.118 | −52.320 | 1.00 | 63.24 | O |
| ATOM | 6681 | N | GLN | B | 223 | 22.721 | −118.119 | −52.355 | 1.00 | 67.84 | N |
| ATOM | 6682 | CA | GLN | B | 223 | 22.675 | −118.699 | −53.775 | 1.00 | 73.25 | C |
| ATOM | 6683 | CB | GLN | B | 223 | 22.074 | −120.083 | −53.928 | 1.00 | 74.05 | C |
| ATOM | 6684 | CG | GLN | B | 223 | 23.051 | −121.165 | −53.524 | 1.00 | 77.35 | C |
| ATOM | 6685 | CD | GLN | B | 223 | 22.429 | −122.142 | −52.568 | 1.00 | 76.75 | C |
| ATOM | 6686 | OE1 | GLN | B | 223 | 21.204 | −122.209 | −52.450 | 1.00 | 76.09 | O |
| ATOM | 6687 | NE2 | GLN | B | 223 | 23.266 | −122.906 | −51.874 | 1.00 | 78.80 | N |
| ATOM | 6688 | C | GLN | B | 223 | 21.949 | −117.706 | −54.674 | 1.00 | 79.55 | C |
| ATOM | 6689 | C | GLN | B | 223 | 22.554 | −117.322 | −55.588 | 1.00 | 80.08 | O |
| ATOM | 6699 | N | GLN | B | 224 | 20.652 | −117.539 | −54.425 | 1.00 | 81.13 | N |
| ATOM | 6691 | CA | GLN | B | 224 | 19.863 | −116.518 | −55.093 | 1.00 | 80.09 | C |
| ATOM | 6692 | CB | GLN | B | 234 | 18.390 | −116.670 | −54.725 | 1.00 | 82.77 | C |
| ATOM | 6693 | CG | GLN | B | 224 | 17.827 | −118.053 | −55.024 | 1.00 | 88.07 | C |
| ATOM | 6694 | CD | GLN | B | 224 | 16.685 | −118.455 | −54.103 | 1.00 | 97.01 | C |
| ATOM | 6695 | OE1 | GLN | B | 224 | 16.794 | −118.379 | −52.878 | 1.00 | 98.43 | O |
| ATOM | 6695 | NE2 | GLN | B | 224 | 15.585 | −118.910 | −54.694 | 1.00 | 100.61 | N |
| ATOM | 6697 | C | GLN | B | 224 | 20.429 | −115.170 | −54.662 | 1.00 | 84.28 | C |
| ATOM | 6698 | O | GLN | B | 224 | 19.945 | −114.544 | −53.719 | 1.00 | 88.72 | O |
| ATOM | 6699 | N | SER | B | 225 | 21.478 | −114.749 | −55.364 | 1.00 | 89.17 | N |
| ATOM | 6700 | CA | SER | B | 225 | 22.331 | −113.617 | −54.979 | 1.00 | 96.78 | C |
| ATOM | 6701 | CB | SER | B | 225 | 23.611 | −113.627 | −55.845 | 1.00 | 101.96 | C |
| ATOM | 6702 | OG | SER | B | 225 | 24.790 | −113.359 | −55.090 | 1.00 | 96.73 | O |
| ATOM | 6703 | C | SER | B | 225 | 21.609 | −112.262 | −55.074 | 1.00 | 92.00 | C |
| ATOM | 6704 | O | SER | B | 225 | 22.221 | −111.195 | −54.917 | 1.00 | 90.50 | O |
| ATOM | 6705 | N | GLU | B | 226 | 20.303 | −112.324 | −55.325 | 1.00 | 91.48 | N |
| ATOM | 6706 | CA | GLU | B | 226 | 19.461 | −111.135 | −55.470 | 1.00 | 91.04 | C |
| ATOM | 6707 | CB | GLU | B | 226 | 18.772 | −111.101 | −56.847 | 1.00 | 97.84 | C |
| ATOM | 6708 | CG | GLU | B | 226 | 19.515 | −111.791 | −57.988 | 1.00 | 107.67 | C |
| ATOM | 6709 | CD | GLU | B | 226 | 19.234 | −113.292 | −58.078 | 1.00 | 117.02 | C |
| ATOM | 6710 | OE1 | GLU | B | 226 | 18.763 | −113.901 | −57.083 | 1.00 | 109.05 | O |
| ATOM | 6711 | OE2 | GLU | B | 226 | 19.496 | −113.871 | −59.158 | 1.00 | 123.34 | O |
| ATOM | 6712 | C | GLU | B | 226 | 18.399 | −111.084 | −54.367 | 1.00 | 82.76 | C |
| ATOM | 6713 | O | GLU | B | 226 | 17.570 | −110.161 | −54.333 | 1.00 | 83.85 | O |
| ATOM | 6714 | N | VAL | B | 227 | 18.410 | −112.083 | −53.482 | 1.00 | 70.57 | N |
| ATOM | 6715 | CA | VAL | B | 227 | 17.475 | −112.106 | −52.355 | 1.00 | 67.27 | C |
| ATOM | 6716 | CB | VAL | B | 227 | 16.483 | −113.317 | −52.366 | 1.00 | 66.65 | C |
| ATOM | 6717 | CG1 | VAL | B | 227 | 16.128 | −113.764 | −53.783 | 1.00 | 66.31 | C |
| ATOM | 6718 | CG2 | VAL | B | 227 | 17.012 | −114.487 | −51.363 | 1.00 | 65.69 | C |
| ATOM | 6719 | C | VAL | B | 227 | 18.177 | −111.985 | −51.001 | 1.00 | 64.13 | C |
| ATOM | 6720 | O | VAL | B | 227 | 19.274 | −112.508 | −50.790 | 1.00 | 62.39 | O |
| ATOM | 6721 | N | PHE | B | 228 | 17.542 | −111.259 | −50.092 | 1.00 | 63.21 | N |
| ATOM | 6722 | CA | PHE | B | 228 | 17.995 | −111.229 | −48.713 | 1.00 | 59.74 | C |
| ATOM | 6723 | CB | PHE | B | 228 | 18.866 | −110.004 | −48.437 | 1.00 | 57.20 | C |
| ATOM | 6724 | CG | PHE | B | 228 | 18.151 | −108.682 | −48.569 | 1.00 | 59.99 | C |
| ATOM | 6725 | CD1 | PHE | B | 228 | 18.027 | −108.057 | −49.804 | 1.00 | 62.80 | C |
| ATOM | 6726 | CE1 | PHE | B | 228 | 17.387 | −106.827 | −49.916 | 1.00 | 60.93 | C |
| ATOM | 6727 | CZ | PHE | B | 228 | 16.884 | −106.203 | −48.789 | 1.00 | 59.44 | C |
| ATOM | 6728 | CE2 | PHE | B | 228 | 17.011 | −106.805 | −47.553 | 1.00 | 56.54 | C |
| ATOM | 6729 | CD2 | PHE | B | 228 | 17.650 | −108.029 | −47.445 | 1.00 | 60.45 | C |
| ATOM | 6730 | C | PHE | B | 228 | 16.814 | −111.285 | −47.769 | 1.00 | 55.67 | C |
| ATOM | 6731 | O | PHE | B | 228 | 15.694 | −110.978 | −48.161 | 1.00 | 55.04 | O |
| ATOM | 6732 | N | VAL | B | 229 | 17.070 | −111.720 | −48.541 | 1.00 | 51.35 | N |
| ATOM | 6733 | CA | VAL | B | 229 | 16.113 | −111.556 | −45.453 | 1.00 | 58.31 | C |
| ATOM | 6734 | CB | VAL | B | 229 | 16.001 | −112.821 | −44.570 | 1.00 | 59.88 | C |
| ATOM | 6735 | CG1 | VAL | B | 229 | 15.326 | −113.950 | −45.336 | 1.00 | 60.26 | C |
| ATOM | 6736 | CC2 | VAL | B | 229 | 17.368 | −113.242 | −44.031 | 1.00 | 55.68 | C |
| ATOM | 6737 | C | VAL | B | 229 | 16.500 | −110.360 | −44.578 | 1.00 | 57.06 | C |
| ATOM | 6738 | O | VAL | B | 229 | 17.678 | −110.005 | −44.483 | 1.00 | 53.72 | O |
| ATOM | 6739 | N | PHE | B | 230 | 15.508 | −109.726 | −43.958 | 1.00 | 57.98 | N |
| ATOM | 6740 | CA | PHE | B | 230 | 15.797 | −108.768 | −42.891 | 1.00 | 58.30 | C |
| ATOM | 6741 | CB | PHE | B | 230 | 15.851 | −107.322 | −43.403 | 1.00 | 61.76 | C |
| ATOM | 6742 | CG | PHE | B | 230 | 14.510 | −106.660 | −43.535 | 1.00 | 63.31 | C |
| ATOM | 6743 | CD1 | PHE | B | 230 | 13.757 | −106.807 | −44.695 | 1.00 | 63.87 | C |
| ATOM | 6744 | CE1 | PHE | B | 230 | 12.528 | −106.184 | −44.828 | 1.00 | 65.25 | C |
| ATOM | 6745 | CZ | PHE | B | 230 | 12.038 | −105.394 | −43.793 | 1.00 | 63.13 | C |
| ATOM | 6748 | CZ2 | PHE | B | 230 | 12.785 | −105.233 | −42.636 | 1.00 | 63.55 | C |
| ATOM | 6747 | CD2 | PHE | B | 230 | 14.011 | −105.859 | −42.514 | 1.00 | 61.43 | C |
| ATOM | 6748 | C | PHE | B | 230 | 14.872 | −108.912 | −41.696 | 1.00 | 51.62 | C |
| ATOM | 6749 | O | PHE | B | 230 | 13.699 | −109.266 | −41.834 | 1.00 | 62.33 | O |
| ATOM | 6750 | N | GLY | B | 231 | 15.419 | −108.667 | −40.517 | 1.00 | 43.63 | N |
| ATOM | 6751 | CA | GLY | B | 231 | 14.654 | −108.795 | −39.295 | 1.00 | 45.82 | C |
| ATOM | 6752 | C | GLY | B | 231 | 14.389 | −107.421 | −38.742 | 1.00 | 43.84 | C |
| ATOM | 6753 | O | GLY | B | 231 | 15.204 | −106.523 | −38.893 | 1.00 | 43.45 | O |
| ATOM | 6754 | N | ASP | B | 232 | 13.251 | −107.236 | −38.096 | 1.00 | 43.91 | N |
| ATOM | 6755 | A | ASP | B | 232 | 12.964 | −105.928 | −37.533 | 1.00 | 43.90 | C |
| ATOM | 6756 | CA | ASP | B | 232 | 11.697 | −105.310 | −38.179 | 1.00 | 43.53 | C |
| ATOM | 6757 | CB | ASP | B | 232 | 10.410 | −106.038 | −37.802 | 1.00 | 51.84 | C |
| ATOM | 6758 | OD1 | ASP | B | 23 | 10.440 | −106.913 | −36.903 | 1.00 | 54.37 | O |
| ATOM | 6759 | OD2 | ASP | B | 232 | 9.350 | −105.721 | −38.406 | 1.00 | 54.57 | O |
| ATOM | 6760 | C | ASP | B | 232 | 12.913 | −105.937 | −36.003 | 1.00 | 43.32 | C |
| ATOM | 6761 | O | ASP | B | 232 | 12.972 | −106.994 | −35.366 | 1.00 | 42.62 | O |
| ATOM | 6762 | N | GLU | B | 233 | 12.782 | −104.109 | −35.426 | 1.00 | 46.16 | N |
| ATOM | 6763 | CA | GLU | B | 233 | 12.708 | −104.185 | −33.971 | 1.00 | 49.17 | C |
| ATOM | 6764 | CB | GLU | B | 233 | 12.836 | −103.115 | −33.614 | 1.00 | 50.41 | C |
| ATOM | 6765 | CG | GLU | B | 233 | 11.615 | −102.110 | −34.031 | 1.00 | 50.69 | C |
| ATOM | 6766 | CD | GLU | B | 233 | 11.801 | −100.827 | −33.821 | 1.00 | 54.72 | C |
| ATOM | 6767 | OE1 | GLU | B | 233 | 12.702 | −100.445 | −33.021 | 1.00 | 54.40 | O |
| ATOM | 6768 | OE2 | GLU | B | 233 | 11.047 | −100.055 | −34.469 | 1.00 | 53.53 | O |
| ATOM | 6769 | C | GLU | B | 233 | 11.418 | −105.329 | −33.354 | 1.00 | 50.69 | C |
| ATOM | 6770 | O | GLU | B | 233 | 11.262 | −105.095 | −32.136 | 1.00 | 53.21 | O |
| ATOM | 6771 | N | ASN | B | 234 | 10.505 | −105.025 | −34.183 | 1.00 | 47.41 | N |
| ATOM | 6772 | CA | ASN | B | 234 | 9.247 | −106.255 | −33.679 | 1.00 | 48.42 | C |
| ATOM | 6773 | CB | ASN | B | 234 | 8.052 | −105.102 | −34.389 | 1.00 | 49.25 | C |
| ATOM | 6774 | CG | ASN | B | 234 | 6.777 | −105.195 | −33.574 | 1.00 | 53.44 | C |
| ATOM | 6775 | OD1 | ASN | B | 234 | 6.796 | −105.433 | −32.345 | 1.00 | 51.90 | O |
| ATOM | 6776 | ND2 | ASN | B | 234 | 5.664 | −105.172 | −34.245 | 1.00 | 50.61 | N |
| ATOM | 6777 | C | ASN | B | 234 | 9.140 | −107.869 | −33.769 | 1.00 | 48.05 | C |
| ATOM | 6778 | O | ASN | B | 234 | 8.116 | −108.235 | −33.413 | 1.00 | 44.76 | O |
| ATOM | 6779 | N | GLY | B | 235 | 10.196 | −108.124 | −34.254 | 1.00 | 49.94 | N |
| ATOM | 6780 | CA | GLY | B | 235 | 10.278 | −109.387 | −34.202 | 1.00 | 47.14 | C |
| ATOM | 6781 | C | GLY | B | 235 | 9.903 | −110.467 | −35.499 | 1.00 | 46.68 | C |
| ATOM | 6782 | O | GLY | B | 235 | 9.784 | −111.497 | −35.567 | 1.00 | 44.02 | O |
| ATOM | 6783 | N | THR | B | 236 | 9.743 | −109.447 | −36.528 | 1.00 | 45.46 | N |
| ATOM | 6784 | CA | THR | B | 236 | 9.355 | −110.301 | −37.835 | 1.00 | 48.24 | C |
| ATOM | 6785 | CB | THR | B | 236 | 8.334 | −109.330 | −38.452 | 1.00 | 49.18 | C |
| ATOM | 6786 | OG1 | THR | B | 236 | 7.145 | −109.366 | −37.660 | 1.00 | 48.92 | O |
| ATOM | 6787 | CG2 | THR | B | 236 | 8.012 | −109.503 | −39.912 | 1.00 | 46.60 | C |
| ATOM | 6788 | C | THR | B | 236 | 10.559 | −110.341 | −38.783 | 1.00 | 49.02 | C |
| ATOM | 6789 | O | THR | B | 236 | 11.351 | −109.320 | −38.923 | 1.00 | 50.66 | O |
| ATOM | 6790 | N | VAL | B | 237 | 10.672 | −111.427 | −39.330 | 1.00 | 53.51 | N |
| ATOM | 6791 | CA | VAL | B | 237 | 11.653 | −111.330 | −40.354 | 1.00 | 53.01 | C |
| ATOM | 6792 | CB | VAL | B | 237 | 12.227 | −113.244 | −40.145 | 1.00 | 50.20 | C |
| ATOM | 6793 | CG1 | VAL | B | 237 | 13.006 | −113.610 | −41.376 | 1.00 | 30.16 | C |
| ATOM | 6794 | CG2 | VAL | B | 237 | 13.089 | −113.364 | −38.887 | 1.00 | 47.26 | C |
| ATOM | 6795 | C | VAL | B | 237 | 10.940 | −111.645 | −41.697 | 1.00 | 54.01 | C |
| ATOM | 6796 | O | VAL | B | 237 | 9.818 | −112.366 | −41.855 | 1.00 | 53.00 | O |
| ATOM | 6797 | N | SER | B | 238 | 11.586 | −110.971 | −42.645 | 1.00 | 51.79 | N |
| ATOM | 6798 | CA | SER | B | 238 | 11.000 | −110.327 | −43.949 | 1.00 | 54.11 | C |
| ATOM | 6799 | CB | SER | B | 238 | 10.470 | −109.301 | −44.031 | 1.00 | 53.21 | C |
| ATOM | 6800 | OG | SER | B | 238 | 9.403 | −109.331 | −43.119 | 1.00 | 55.74 | O |
| ATOM | 6801 | C | SER | B | 238 | 12.000 | −110.585 | −45.061 | 1.00 | 60.16 | C |
| ATOM | 6802 | O | SER | B | 238 | 13.206 | −110.364 | −44.902 | 1.00 | 67.46 | O |
| ATOM | 6803 | N | LEU | B | 239 | 11.477 | −111.443 | −46.193 | 1.00 | 62.87 | N |
| ATOM | 6804 | CA | LEU | B | 239 | 12.280 | −111.828 | −47.344 | 1.00 | 62.68 | C |
| ATOM | 6805 | CB | LEU | B | 239 | 11.945 | −113.376 | −47.737 | 1.00 | 63.72 | C |
| ATOM | 6806 | CG | LEU | B | 239 | 12.078 | −113.889 | −49.130 | 1.00 | 67.13 | C |
| ATOM | 6807 | CD1 | LEU | B | 239 | 13.528 | −114.125 | −49.525 | 1.00 | 68.98 | C |
| ATOM | 6808 | CD2 | LEU | B | 239 | 11.321 | −115.303 | −49.124 | 1.00 | 65.40 | C |
| ATOM | 6809 | C | LEU | B | 239 | 12.067 | −110.829 | −48.478 | 1.00 | 59.66 | C |
| ATOM | 6810 | O | LEU | B | 239 | 10.941 | −110.584 | −48.910 | 1.00 | 59.67 | O |
| ATOM | 6811 | N | VAL | B | 240 | 13.167 | −110.227 | −48.915 | 1.00 | 59.98 | N |
| ATOM | 6812 | CA | VAL | B | 240 | 13.160 | −109.233 | −49.972 | 1.00 | 61.21 | C |
| ATOM | 6813 | CB | VAL | B | 240 | 13.824 | −107.330 | −49.512 | 1.00 | 59.21 | C |
| ATOM | 6814 | CG1 | VAL | B | 240 | 13.591 | −106.818 | −50.534 | 1.00 | 56.34 | C |
| ATOM | 6815 | CG2 | VAL | B | 240 | 13.310 | −107.523 | −48.154 | 1.00 | 56.32 | C |
| ATOM | 6816 | C | VAL | B | 240 | 13.902 | −109.700 | −51.229 | 1.00 | 69.67 | C |
| ATOM | 6817 | O | VAL | B | 240 | 14.924 | −110.405 | −51.154 | 1.00 | 65.53 | O |
| ATOM | 6818 | N | ASP | B | 241 | 13.362 | −109.280 | −52.373 | 1.00 | 76.53 | N |
| ATOM | 6819 | CA | ASP | B | 241 | 13.964 | −109.453 | −53.689 | 1.00 | 81.39 | C |
| ATOM | 6820 | CB | ASP | B | 241 | 12.907 | −110.018 | −54.649 | 1.00 | 83.32 | C |
| ATOM | 6821 | CG | ASP | B | 241 | 13.506 | −110.656 | −55.901 | 1.00 | 87.46 | C |
| ATOM | 6822 | OD1 | ASP | B | 241 | 14.253 | −109.976 | −56.636 | 1.00 | 96.81 | O |
| ATOM | 6823 | OD2 | ASP | B | 241 | 13.200 | −111.838 | −56.171 | 1.00 | 78.82 | O |
| ATOM | 6824 | C | ASP | B | 241 | 14.426 | −108.672 | −54.158 | 1.00 | 80.31 | C |
| ATOM | 6825 | O | ASP | B | 241 | 13.662 | −107.106 | −54.093 | 1.00 | 89.48 | O |
| ATOM | 6826 | N | THR | B | 242 | 15.667 | −107.964 | −54.617 | 1.00 | 78.54 | N |
| ATOM | 6827 | CA | THR | B | 242 | 16.153 | −106.683 | −55.143 | 1.00 | 80.15 | C |
| ATOM | 6828 | CB | THR | B | 242 | 17.692 | −106.600 | −55.167 | 1.00 | 78.00 | C |
| ATOM | 6829 | OG1 | THR | B | 242 | 18.230 | −107.664 | −55.966 | 1.00 | 86.54 | O |
| ATOM | 6830 | CG2 | THR | B | 242 | 18.234 | −106.715 | −53.773 | 1.00 | 75.73 | C |
| ATOM | 6831 | C | THR | B | 242 | 15.571 | −106.338 | −56.524 | 1.00 | 81.95 | C |
| ATOM | 6832 | O | THR | B | 242 | 15.244 | −105.178 | −56.782 | 1.00 | 79.10 | O |
| ATOM | 6833 | N | LYS | B | 243 | 15.432 | −107.350 | −57.387 | 1.00 | 89.88 | N |
| ATOM | 6834 | CA | LYS | B | 243 | 14.877 | −107.189 | −58.745 | 1.00 | 100.06 | C |
| ATOM | 6835 | CB | LYS | B | 243 | 14.907 | −108.518 | −59.523 | 1.00 | 99.48 | C |
| ATOM | 6836 | CG | LYS | B | 243 | 16.296 | −109.099 | −59.763 | 1.00 | 100.34 | C |
| ATOM | 6837 | CD | LYS | B | 243 | 17.183 | −108.147 | −60.559 | 1.00 | 99.82 | C |
| ATOM | 6838 | CE | LYS | B | 243 | 18.658 | −108.472 | −60.379 | 1.00 | 100.33 | C |
| ATOM | 6839 | NZ | LYS | B | 243 | 19.086 | −108.440 | −58.948 | 1.00 | 100.66 | N |
| ATOM | 6840 | C | LYS | B | 243 | 13.460 | −106.606 | −58.748 | 1.00 | 106.09 | C |
| ATOM | 6841 | O | LYS | B | 243 | 13.254 | −105.483 | −59.220 | 1.00 | 111.67 | O |
| ATOM | 6842 | N | SER | B | 244 | 12.502 | −107.366 | −58.208 | 1.00 | 111.53 | N |
| ATOM | 6843 | CA | SER | B | 244 | 11.093 | −106.933 | −57.089 | 1.00 | 113.71 | C |
| ATOM | 6844 | CB | SER | B | 244 | 10.161 | −108.133 | −57.856 | 1.00 | 109.94 | C |
| ATOM | 6845 | OG | SER | B | 244 | 10.597 | −108.927 | −56.764 | 1.00 | 101.67 | O |
| ATOM | 6846 | C | SER | B | 244 | 10.882 | −105.864 | −57.008 | 1.00 | 108.68 | C |
| ATOM | 6847 | O | SER | B | 244 | 9.754 | −105.407 | −56.780 | 1.00 | 107.08 | O |
| ATOM | 6848 | N | THR | B | 245 | 11.981 | −105.487 | −56.350 | 1.00 | 102.97 | N |
| ATOM | 6849 | CA | THR | B | 245 | 12.048 | −104.345 | −55.428 | 1.00 | 99.95 | C |
| ATOM | 6850 | CB | THR | B | 245 | 12.533 | −103.051 | −56.171 | 1.00 | 93.88 | C |
| ATOM | 6851 | OG1 | THR | B | 245 | 12.693 | −101.973 | −55.243 | 1.00 | 85.10 | O |
| ATOM | 6852 | CG2 | THR | B | 245 | 11.590 | −102.625 | −57.324 | 1.00 | 91.37 | C |
| ATOM | 6853 | C | THR | B | 245 | 10.792 | −104.169 | −54.525 | 1.00 | 103.43 | C |
| ATOM | 6854 | O | THR | B | 245 | 10.228 | −103.077 | −54.382 | 1.00 | 104.33 | O |
| ATOM | 6855 | N | SER | B | 246 | 10.369 | −105.283 | −53.928 | 1.00 | 105.85 | N |
| ATOM | 6856 | CA | SER | B | 246 | 9.276 | −105.303 | −52.946 | 1.00 | 113.36 | C |
| ATOM | 6857 | CB | SER | B | 246 | 7.904 | −105.167 | −53.624 | 1.00 | 102.38 | C |
| ATOM | 6858 | OG | SER | B | 246 | 6.977 | −104.344 | −52.748 | 1.00 | 91.51 | O |
| ATOM | 6859 | C | SER | B | 246 | 9.331 | −106.568 | −52.069 | 1.00 | 111.88 | C |
| ATOM | 6860 | O | SER | B | 246 | 9.949 | −107.574 | −52.449 | 1.00 | 105.12 | O |
| ATOM | 6861 | N | CYS | B | 247 | 8.686 | −106.502 | −50.900 | 1.00 | 103.39 | N |
| ATOM | 6862 | CA | CYS | B | 247 | 8.718 | −107.579 | −49.905 | 1.00 | 109.16 | C |
| ATOM | 6863 | CB | CYS | B | 247 | 8.358 | −107.051 | −48.514 | 1.00 | 97.52 | C |
| ATOM | 6864 | SG | CYS | B | 247 | 9.574 | −105.880 | −47.868 | 1.00 | 105.12 | S |
| ATOM | 6865 | C | CYS | B | 247 | 7.790 | −108.714 | −50.287 | 1.00 | 95.97 | C |
| ATOM | 6866 | O | CYS | B | 247 | 6.587 | −108.507 | −50.465 | 1.00 | 89.37 | O |
| ATOM | 6867 | N | VAL | B | 248 | 8.364 | −109.912 | −50.407 | 1.00 | 93.90 | N |
| ATOM | 6868 | CA | VAL | B | 248 | 7.609 | −111.084 | −50.845 | 1.00 | 91.19 | C |
| ATOM | 6869 | CB | VAL | B | 248 | 8.437 | −112.062 | −51.764 | 1.00 | 94.15 | C |
| ATOM | 6870 | CG1 | VAL | B | 248 | 9.581 | −111.342 | −52.477 | 1.00 | 90.77 | C |
| ATOM | 6871 | CG2 | VAL | B | 248 | 8.975 | −113.274 | −51.018 | 1.00 | 91.25 | C |
| ATOM | 6872 | C | VAL | B | 248 | 6.919 | −111.747 | −49.641 | 1.00 | 89.74 | C |
| ATOM | 6873 | O | VAL | B | 248 | 5.691 | −111.668 | −49.506 | 1.00 | 89.01 | O |
| ATOM | 6874 | N | LEU | B | 249 | 7.708 | −112.347 | −48.753 | 1.00 | 81.63 | N |
| ATOM | 6875 | CA | LEU | B | 249 | 7.178 | −113.071 | −47.598 | 1.00 | 75.99 | C |
| ATOM | 6876 | CB | LEU | B | 249 | 7.627 | −114.530 | −47.649 | 1.00 | 73.75 | C |
| ATOM | 6877 | CG | LEU | B | 249 | 6.656 | −115.608 | −48.127 | 1.00 | 81.52 | C |
| ATOM | 6878 | CD1 | LEU | B | 249 | 6.057 | −115.296 | −49.497 | 1.00 | 81.91 | C |
| ATOM | 6879 | CD2 | LEU | B | 249 | 7.365 | −116.956 | −48.126 | 1.00 | 73.65 | C |
| ATOM | 6880 | C | LEU | B | 249 | 7.619 | −112.451 | −46.277 | 1.00 | 71.85 | C |
| ATOM | 6881 | O | LEU | B | 249 | 8.628 | −111.744 | −46.219 | 1.00 | 65.92 | O |
| ATOM | 6882 | N | SER | B | 250 | 6.858 | −112.737 | −45.221 | 1.00 | 71.59 | N |
| ATOM | 6883 | CA | SER | B | 250 | 7.163 | −112.271 | −43.871 | 1.00 | 69.11 | C |
| ATOM | 6884 | CE | SER | B | 250 | 6.689 | −110.829 | −43.674 | 1.00 | 71.29 | C |
| ATOM | 6885 | OG | SER | B | 250 | 5.272 | −110.745 | −43.679 | 1.00 | 74.14 | O |
| ATOM | 6886 | C | SER | B | 250 | 6.494 | −113.169 | −42.841 | 1.00 | 69.88 | C |
| ATOM | 6887 | O | SER | B | 250 | 5.409 | −113.698 | −43.071 | 1.00 | 70.66 | O |
| ATOM | 6888 | N | SER | B | 251 | 7.134 | −113.324 | −41.691 | 1.00 | 70.27 | N |
| ATOM | 6889 | CA | SER | B | 251 | 6.551 | −114.099 | −40.610 | 1.00 | 65.57 | C |
| ATOM | 6890 | CB | SER | B | 251 | 7.137 | −115.508 | −40.610 | 1.00 | 63.81 | C |
| ATOM | 6891 | OG | SER | B | 251 | 6.267 | −116.429 | −39.978 | 1.00 | 64.85 | O |
| ATOM | 6892 | C | SER | B | 251 | 6.828 | −113.215 | −39.275 | 1.00 | 63.43 | C |
| ATOM | 6893 | O | SER | B | 251 | 7.900 | −112.832 | −39.093 | 1.00 | 59.77 | O |
| ATOM | 6894 | N | ALA | B | 252 | 5.864 | −113.290 | −38.352 | 1.00 | 61.97 | N |
| ATOM | 6895 | CA | ALA | B | 252 | 6.097 | −113.095 | −36.948 | 1.00 | 62.07 | C |
| ATOM | 6896 | CB | ALA | B | 252 | 4.782 | −112.143 | −36.258 | 1.00 | 59.40 | C |
| ATOM | 6897 | C | ALA | B | 252 | 6.827 | −114.219 | −36.198 | 1.00 | 61.14 | C |
| ATOM | 6898 | O | ALA | B | 252 | 6.192 | −115.119 | −35.636 | 1.00 | 62.04 | O |
| ATOM | 6899 | N | VAL | B | 253 | 8.163 | −114.170 | −36.215 | 1.00 | 58.05 | N |
| ATOM | 6900 | CA | VAL | B | 253 | 9.000 | −115.248 | −35.670 | 1.00 | 50.85 | C |
| ATOM | 6901 | CB | VAL | B | 253 | 10.377 | −115.336 | −36.381 | 1.00 | 50.52 | C |
| ATOM | 6902 | CG1 | VAL | B | 253 | 11.205 | −116.526 | −35.878 | 1.00 | 45.97 | C |
| ATOM | 6903 | CG2 | VAL | B | 253 | 10.195 | −115.429 | −37.883 | 1.00 | 51.48 | C |
| ATOM | 6904 | C | VAL | B | 253 | 9.229 | −115.041 | −34.185 | 1.00 | 50.29 | C |
| ATOM | 6905 | O | VAL | B | 253 | 9.101 | −115.974 | −33.396 | 1.00 | 48.47 | O |
| ATOM | 6906 | N | HIS | B | 254 | 9.566 | −113.811 | −33.811 | 1.00 | 51.70 | N |
| ATOM | 6907 | CA | HIS | B | 254 | 9.976 | −113.517 | −32.447 | 1.00 | 51.57 | C |
| ATOM | 6908 | CB | HIS | B | 254 | 11.419 | −113.041 | −32.430 | 1.00 | 53.85 | C |
| ATOM | 6909 | CG | HIS | B | 254 | 12.415 | −114.089 | −32.816 | 1.00 | 53.34 | C |
| ATOM | 6910 | ND1 | HIS | B | 254 | 12.767 | −115.123 | −31.979 | 1.00 | 54.14 | N |
| ATOM | 6911 | CE1 | HIS | B | 254 | 13.694 | −115.864 | −32.562 | 1.00 | 53.54 | C |
| ATOM | 6912 | NE2 | HIS | B | 254 | 13.968 | −115.335 | −33.740 | 1.00 | 52.26 | N |
| ATOM | 6913 | CD2 | HIS | B | 254 | 13.185 | −114.221 | −33.921 | 1.00 | 50.52 | C |
| ATOM | 6914 | C | HIS | B | 254 | 9.109 | −112.469 | −31.769 | 1.00 | 52.73 | C |
| ATOM | 6915 | O | HIS | B | 254 | 8.413 | −111.691 | −32.432 | 1.00 | 53.45 | O |
| ATOM | 6916 | N | SER | B | 255 | 9.184 | −112.457 | −30.439 | 1.00 | 54.62 | N |
| ATOM | 6917 | CA | SER | B | 255 | 8.375 | −111.592 | −29.584 | 1.00 | 58.38 | C |
| ATOM | 6918 | CB | SER | B | 255 | 7.840 | −112.381 | −28.380 | 1.00 | 59.19 | C |
| ATOM | 6919 | OG | SER | B | 255 | 8.881 | −112.718 | −27.463 | 1.00 | 62.63 | O |
| ATOM | 6920 | C | SER | B | 255 | 9.203 | −110.400 | −29.106 | 1.00 | 61.62 | C |
| ATOM | 6921 | O | SER | B | 255 | 8.747 | −109.593 | −28.290 | 1.00 | 71.81 | O |
| ATOM | 6922 | N | GLN | B | 256 | 10.430 | −110.320 | −29.602 | 1.00 | 55.43 | N |
| ATOM | 6923 | CA | GLN | B | 256 | 11.331 | −109.209 | −29.339 | 1.00 | 54.99 | C |
| ATOM | 6924 | CB | GLN | B | 256 | 12.273 | −109.502 | −28.164 | 1.00 | 58.46 | C |
| ATOM | 6925 | CG | GLN | B | 256 | 11.611 | −109.745 | −26.814 | 1.00 | 62.71 | C |
| ATOM | 6926 | CD | GLN | B | 256 | 11.877 | −108.632 | −25.829 | 1.00 | 67.29 | C |
| ATOM | 6927 | OE1 | GLN | B | 256 | 11.409 | −107.509 | −26.003 | 1.00 | 66.82 | O |
| ATOM | 6928 | NE2 | GLN | B | 256 | 12.639 | −108.538 | −24.784 | 1.00 | 69.63 | N |
| ATOM | 6929 | C | GLN | B | 256 | 12.157 | −109.026 | −30.603 | 1.00 | 51.65 | C |
| ATOM | 6930 | O | GLN | B | 256 | 12.054 | −109.807 | −31.550 | 1.00 | 50.84 | O |
| ATOM | 6931 | N | CYS | B | 257 | 12.994 | −108.007 | −30.602 | 1.00 | 47.03 | N |
| ATOM | 6932 | CA | CYS | B | 257 | 13.787 | −107.663 | −31.761 | 1.00 | 51.10 | C |
| ATOM | 6933 | CB | CYS | B | 257 | 14.634 | −106.458 | −31.380 | 1.00 | 56.00 | C |
| ATOM | 6934 | SG | CYS | B | 257 | 16.378 | −106.613 | −31.699 | 1.00 | 68.39 | S |
| ATOM | 6935 | C | CYS | B | 257 | 14.632 | −108.842 | −32.316 | 1.00 | 49.77 | C |
| ATOM | 6936 | O | CYS | B | 257 | 15.156 | −109.648 | −31.547 | 1.00 | 47.59 | O |
| ATOM | 6937 | N | VAL | B | 258 | 14.721 | −108.951 | −33.645 | 1.00 | 45.92 | N |
| ATOM | 6938 | CA | VAL | B | 258 | 15.546 | −109.974 | −34.324 | 1.00 | 46.44 | C |
| ATOM | 6939 | CB | VAL | B | 258 | 14.946 | −110.354 | −35.697 | 1.00 | 44.64 | C |
| ATOM | 6940 | CG1 | VAL | B | 258 | 15.716 | −111.511 | −36.312 | 1.00 | 43.04 | C |
| ATOM | 6941 | CG2 | VAL | B | 258 | 13.461 | −110.701 | −35.567 | 1.00 | 44.78 | C |
| ATOM | 6942 | C | VAL | B | 258 | 16.994 | −109.495 | −34.541 | 1.00 | 48.83 | C |
| ATOM | 6943 | O | VAL | B | 258 | 17.254 | −108.689 | −35.437 | 1.00 | 53.78 | O |
| ATOM | 6944 | N | THR | B | 259 | 17.931 | −109.986 | −33.735 | 1.00 | 50.45 | N |
| ATOM | 6945 | CA | THR | B | 259 | 19.323 | −109.484 | −33.780 | 1.00 | 55.42 | C |
| ATOM | 6946 | CB | THR | B | 259 | 19.999 | −109.506 | −32.389 | 1.00 | 58.87 | C |
| ATOM | 6947 | OG1 | THR | B | 259 | 19.557 | −110.657 | −31.662 | 1.00 | 60.13 | O |
| ATOM | 6948 | CG2 | THR | B | 259 | 19.654 | −108.229 | −31.592 | 1.00 | 59.10 | C |
| ATOM | 6949 | C | THR | B | 259 | 20.255 | −110.158 | −34.800 | 1.00 | 53.34 | C |
| ATOM | 6950 | O | THR | B | 259 | 21.277 | −109.583 | −35.184 | 1.00 | 56.21 | O |
| ATOM | 6951 | N | GLY | B | 260 | 19.918 | −111.368 | −35.229 | 1.00 | 50.40 | N |
| ATOM | 6952 | CA | GLY | B | 260 | 20.735 | −112.042 | −36.225 | 1.00 | 48.80 | C |
| ATOM | 6953 | C | GLY | B | 260 | 19.994 | −113.015 | −37.114 | 1.00 | 49.37 | C |
| ATOM | 6954 | O | GLY | B | 260 | 19.029 | −113.673 | −36.681 | 1.00 | 49.37 | O |
| ATOM | 6955 | N | LEU | B | 261 | 20.449 | −113.099 | −38.364 | 1.00 | 48.56 | N |
| ATOM | 6956 | CA | LEU | B | 261 | 19.909 | −114.059 | −39.333 | 1.00 | 50.14 | C |
| ATOM | 6957 | CB | LEU | B | 261 | 18.964 | −113.360 | −40.307 | 1.00 | 46.11 | C |
| ATOM | 6958 | CG | LEU | B | 261 | 17.707 | −112.709 | −39.712 | 1.00 | 44.38 | C |
| ATOM | 6959 | CD1 | LEU | B | 261 | 17.168 | −111.620 | −40.630 | 1.00 | 42.83 | C |
| ATOM | 6960 | CD2 | LEU | B | 261 | 16.639 | −113.753 | −39.411 | 1.00 | 45.14 | C |
| ATOM | 6961 | C | LEU | B | 261 | 21.038 | −114.798 | −40.081 | 1.00 | 54.62 | C |
| ATOM | 6962 | O | LEU | B | 261 | 21.886 | −114.176 | −40.742 | 1.00 | 53.73 | O |
| ATOM | 6963 | N | VAL | B | 262 | 21.054 | −116.124 | −39.951 | 1.00 | 54.69 | N |
| ATOM | 6964 | CA | VAL | B | 262 | 22.133 | −116.942 | −40.509 | 1.00 | 59.88 | C |
| ATOM | 6965 | CB | VAL | B | 262 | 23.060 | −117.515 | −39.405 | 1.00 | 56.78 | C |
| ATOM | 6966 | CG1 | VAL | B | 262 | 24.209 | −118.277 | −40.028 | 1.00 | 48.91 | C |
| ATOM | 6967 | CG2 | VAL | B | 262 | 23.592 | −116.414 | −38.491 | 1.00 | 53.30 | C |
| ATOM | 6968 | C | VAL | B | 262 | 21.583 | −118.096 | −41.355 | 1.00 | 63.67 | C |
| ATOM | 6969 | O | VAL | B | 262 | 20.852 | −118.958 | −40.852 | 1.00 | 61.69 | O |
| ATOM | 6970 | N | PHE | B | 263 | 21.946 | −118.104 | −42.639 | 1.00 | 64.33 | N |
| ATOM | 6971 | CA | PHE | B | 263 | 21.582 | −119.193 | −43.551 | 1.00 | 61.04 | C |
| ATOM | 6972 | CB | PHE | B | 263 | 21.601 | −118.710 | −45.007 | 1.00 | 56.53 | C |
| ATOM | 6973 | CG | PHE | B | 263 | 20.366 | −117.964 | −45.422 | 1.00 | 49.58 | C |
| ATOM | 6974 | CD1 | PHE | B | 263 | 19.140 | −118.620 | −45.543 | 1.00 | 50.36 | C |
| ATOM | 6975 | CE1 | PHE | B | 263 | 17.990 | −117.924 | −45.939 | 1.00 | 48.09 | C |
| ATOM | 6976 | CZ | PHE | B | 263 | 18.062 | −116.563 | −46.218 | 1.00 | 44.10 | C |
| ATOM | 6977 | CE2 | PHE | B | 263 | 19.280 | −115.902 | −46.104 | 1.00 | 46.05 | C |
| ATOM | 6978 | CD2 | PHE | B | 263 | 20.423 | −116.604 | −45.710 | 1.00 | 48.96 | C |
| ATOM | 6979 | C | PHE | B | 263 | 22.500 | −120.408 | −43.385 | 1.00 | 59.97 | C |
| ATOM | 6980 | O | PHE | B | 263 | 23.704 | −120.264 | −43.159 | 1.00 | 62.68 | O |
| ATOM | 6981 | N | SER | B | 264 | 21.925 | −121.604 | −43.498 | 1.00 | 62.69 | N |
| ATOM | 6982 | CA | SER | B | 264 | 22.705 | −122.842 | −43.424 | 1.00 | 63.75 | C |
| ATOM | 6983 | CB | SER | B | 264 | 21.787 | −124.039 | −43.152 | 1.00 | 65.30 | C |
| ATOM | 6984 | OG | SER | B | 264 | 21.044 | −124.393 | −44.255 | 1.00 | 64.51 | O |
| ATOM | 6985 | C | SER | B | 264 | 23.529 | −123.065 | −44.699 | 1.00 | 69.70 | C |
| ATOM | 6986 | O | SER | B | 264 | 23.239 | −122.454 | −45.735 | 1.00 | 57.05 | O |
| ATOM | 6987 | N | PRO | B | 265 | 24.558 | −123.934 | −44.628 | 1.00 | 63.73 | N |
| ATOM | 6988 | CA | PRO | B | 265 | 25.349 | −124.248 | −45.829 | 1.00 | 67.47 | C |
| ATOM | 6989 | CB | PRO | B | 265 | 26.625 | −124.907 | −45.263 | 1.00 | 64.33 | C |
| ATOM | 6990 | CG | PRO | B | 265 | 26.574 | −124.710 | −43.777 | 1.00 | 63.19 | C |
| ATOM | 6991 | CD | PRO | B | 265 | 25.114 | −124.598 | −43.437 | 1.00 | 63.78 | C |
| ATOM | 6992 | C | PRO | B | 265 | 24.634 | −125.194 | −46.820 | 1.00 | 71.48 | C |
| ATOM | 6993 | O | PRO | B | 265 | 25.042 | −125.269 | −47.981 | 1.00 | 63.39 | O |
| ATOM | 6994 | N | HIS | B | 266 | 23.587 | −125.891 | −46.359 | 1.00 | 75.64 | N |
| ATOM | 6995 | CA | HIS | B | 266 | 22.714 | −126.753 | −47.190 | 1.00 | 75.31 | C |
| ATOM | 6996 | CB | HIS | B | 266 | 21.360 | −126.965 | −46.496 | 1.00 | 73.92 | C |
| ATOM | 6997 | CG | HIS | B | 266 | 21.436 | −127.768 | −45.234 | 1.00 | 83.03 | C |
| ATOM | 6998 | ND1 | HIS | B | 266 | 21.033 | −129.084 | −45.166 | 1.00 | 85.03 | N |
| ATOM | 6999 | CE1 | HIS | B | 266 | 21.204 | −129.534 | −43.935 | 1.00 | 85.44 | C |
| ATOM | 7000 | NE2 | HIS | B | 266 | 21.699 | −128.555 | −43.201 | 1.00 | 83.64 | N |
| ATOM | 7001 | CE2 | HIS | B | 266 | 21.857 | −127.440 | −43.990 | 1.00 | 87.51 | C |
| ATOM | 7002 | C | HIS | B | 266 | 22.431 | −126.254 | −48.612 | 1.00 | 83.06 | C |
| ATOM | 7003 | O | HIS | B | 266 | 22.432 | −125.036 | −48.867 | 1.00 | 83.46 | O |
| ATOM | 7004 | N | SER | B | 267 | 22.157 | −127.206 | −49.516 | 1.00 | 84.83 | N |
| ATOM | 7005 | CA | SER | B | 267 | 21.780 | −126.917 | −50.913 | 1.00 | 82.97 | C |
| ATOM | 7006 | CB | SER | B | 267 | 21.452 | −128.206 | −51.677 | 1.00 | 81.45 | C |
| ATOM | 7007 | OG | SER | B | 267 | 22.628 | −128.825 | −52.159 | 1.00 | 73.15 | O |
| ATOM | 7008 | C | SER | B | 267 | 20.606 | −125.944 | −50.985 | 1.00 | 89.96 | C |
| ATOM | 7009 | O | SER | B | 267 | 20.748 | −124.824 | −51.485 | 1.00 | 73.52 | O |
| ATOM | 7010 | N | VAL | B | 268 | 19.447 | −126.378 | −50.496 | 1.00 | 81.45 | N |
| ATOM | 7011 | CA | VAL | B | 268 | 18.347 | −125.453 | −50.223 | 1.00 | 83.90 | C |
| ATOM | 7012 | CB | VAL | B | 268 | 16.973 | −126.019 | −50.668 | 1.00 | 85.21 | C |
| ATOM | 7013 | CG1 | VAL | B | 268 | 16.732 | −127.423 | −50.106 | 1.00 | 91.60 | C |
| ATOM | 7014 | CG2 | VAL | B | 268 | 15.836 | −125.050 | −50.335 | 1.00 | 85.13 | C |
| ATOM | 7015 | C | VAL | B | 268 | 18.445 | −125.085 | −48.728 | 1.00 | 81.84 | C |
| ATOM | 7016 | O | VAL | B | 268 | 18.232 | −125.938 | −47.856 | 1.00 | 79.42 | O |
| ATOM | 7017 | N | PRO | B | 269 | 18.805 | −123.818 | −48.430 | 1.00 | 77.70 | N |
| ATOM | 7018 | CA | PRO | B | 269 | 19.321 | −123.516 | −47.104 | 1.00 | 75.82 | C |
| ATOM | 7019 | CB | PRO | B | 269 | 20.235 | −122.311 | −47.354 | 1.00 | 73.15 | C |
| ATOM | 7020 | CG | PRO | B | 269 | 19.793 | −121.729 | −48.665 | 1.00 | 75.81 | C |
| ATOM | 7021 | CD | PRO | B | 269 | 18.697 | −122.592 | −49.238 | 1.00 | 77.47 | C |
| ATOM | 7022 | C | PRO | B | 269 | 18.256 | −123.168 | −46.072 | 1.00 | 73.40 | C |
| ATOM | 7023 | O | PRO | B | 269 | 17.209 | −122.600 | −46.410 | 1.00 | 71.41 | O |
| ATOM | 7024 | N | PHE | B | 270 | 18.546 | −123.536 | −44.826 | 1.00 | 68.59 | N |
| ATOM | 7025 | CA | PHE | B | 270 | 17.748 | −123.151 | −43.672 | 1.00 | 67.87 | C |
| ATOM | 7026 | CB | PHE | B | 270 | 17.872 | −124.190 | −42.560 | 1.00 | 69.00 | C |
| ATOM | 7027 | CG | PHE | B | 270 | 17.433 | −125.563 | −42.960 | 1.00 | 74.77 | C |
| ATOM | 7028 | CD1 | PHE | B | 270 | 16.085 | −125.861 | −43.103 | 1.00 | 75.44 | C |
| ATOM | 7029 | CE1 | PHE | B | 270 | 15.683 | −127.132 | −43.472 | 1.00 | 81.93 | C |
| ATOM | 7030 | CZ | PHE | B | 270 | 16.633 | −128.120 | −43.694 | 1.00 | 83.83 | C |
| ATOM | 7031 | CE2 | PHE | B | 270 | 17.982 | −127.835 | −43.555 | 1.00 | 75.59 | C |
| ATOM | 7032 | CD2 | PHE | B | 270 | 18.374 | −126.564 | −43.190 | 1.00 | 75.90 | C |
| ATOM | 7033 | C | PHE | B | 270 | 18.239 | −121.813 | −43.148 | 1.00 | 63.63 | C |
| ATOM | 7034 | O | PHE | B | 270 | 19.401 | −121.453 | −43.348 | 1.00 | 66.16 | O |
| ATOM | 7035 | N | LEU | B | 271 | 17.347 | −121.098 | −42.468 | 1.00 | 60.91 | N |
| ATOM | 7036 | CA | LEU | B | 271 | 17.648 | −119.796 | −41.899 | 1.00 | 56.42 | C |
| ATOM | 7037 | CB | LEU | B | 271 | 16.699 | −118.749 | −42.463 | 1.00 | 54.17 | C |
| ATOM | 7038 | CG | LEU | B | 271 | 16.955 | −117.339 | −41.945 | 1.00 | 54.00 | C |
| ATOM | 7039 | CD1 | LEU | B | 271 | 18.336 | −116.904 | −42.412 | 1.00 | 50.39 | C |
| ATOM | 7040 | CD2 | LEU | B | 271 | 15.875 | −116.152 | −42.385 | 1.00 | 46.59 | C |
| ATOM | 7041 | C | LEU | B | 271 | 17.497 | −119.834 | −40.393 | 1.00 | 56.83 | C |
| ATOM | 7042 | O | LEU | B | 271 | 16.401 | −120.060 | −39.878 | 1.00 | 56.65 | O |
| ATOM | 7043 | N | ALA | B | 272 | 18.610 | −119.628 | −39.695 | 1.00 | 55.31 | N |
| ATOM | 7044 | CA | ALA | B | 272 | 18.607 | −119.457 | −38.250 | 1.00 | 52.09 | C |
| ATOM | 7045 | CB | ALA | B | 272 | 19.972 | −119.774 | −37.680 | 1.00 | 48.67 | C |
| ATOM | 7046 | C | ALA | B | 272 | 18.197 | −118.019 | −37.903 | 1.00 | 55.12 | C |
| ATOM | 7047 | O | ALA | B | 272 | 18.675 | −117.647 | −38.495 | 1.00 | 50.38 | O |
| ATOM | 7048 | N | SER | B | 273 | 17.287 | −117.502 | −36.945 | 1.00 | 59.82 | N |
| ATOM | 7049 | CA | SER | B | 273 | 16.788 | −116.613 | −36.497 | 1.00 | 62.85 | C |
| ATOM | 7050 | CB | SER | B | 273 | 15.295 | −116.508 | −36.806 | 1.00 | 62.34 | C |
| ATOM | 7051 | OG | SER | B | 273 | 14.847 | −115.175 | −36.675 | 1.00 | 70.42 | O |
| ATOM | 7052 | C | SER | B | 273 | 17.038 | −116.494 | −34.994 | 1.00 | 59.97 | C |
| ATOM | 7053 | O | SER | B | 273 | 16.632 | −117.370 | −34.219 | 1.00 | 61.35 | O |
| ATOM | 7054 | N | LEU | B | 274 | 17.724 | −115.432 | −34.585 | 1.00 | 53.66 | N |
| ATOM | 7055 | CA | LEU | B | 274 | 18.043 | −115.248 | −33.168 | 1.00 | 50.50 | C |
| ATOM | 7056 | CB | LEU | B | 274 | 19.523 | −115.489 | −32.885 | 1.00 | 47.11 | C |
| ATOM | 7057 | CG | LEU | B | 274 | 20.524 | −114.676 | −33.692 | 1.00 | 46.74 | C |
| ATOM | 7058 | CD1 | LEU | B | 274 | 21.585 | −114.125 | −32.771 | 1.00 | 47.85 | C |
| ATOM | 7059 | CD2 | LEU | B | 274 | 21.164 | −115.520 | −34.772 | 1.00 | 46.89 | C |
| ATOM | 7060 | C | LEU | B | 274 | 17.583 | −113.897 | −32.623 | 1.00 | 51.49 | C |
| ATOM | 7061 | O | LEU | B | 274 | 17.612 | −112.886 | −33.320 | 1.00 | 52.42 | O |
| ATOM | 7062 | N | SER | B | 275 | 17.164 | −113.889 | −31.361 | 1.00 | 53.45 | N |
| ATOM | 7063 | CA | SER | B | 275 | 16.438 | −112.753 | −30.821 | 1.00 | 51.27 | C |
| ATOM | 7064 | CB | SER | B | 275 | 14.936 | −112.976 | −31.022 | 1.00 | 49.79 | C |
| ATOM | 7065 | OG | SER | B | 275 | 14.179 | −111.961 | −30.403 | 1.00 | 50.15 | O |
| ATOM | 7066 | C | SER | B | 275 | 16.724 | −112.444 | −29.355 | 1.00 | 51.05 | C |
| ATOM | 7067 | O | SER | B | 275 | 17.184 | −113.296 | −28.592 | 1.00 | 50.80 | O |
| ATOM | 7068 | N | GLU | B | 276 | 16.424 | −111.203 | −28.978 | 1.00 | 49.86 | N |
| ATOM | 7069 | CA | GLU | B | 276 | 16.444 | −110.770 | −27.590 | 1.00 | 46.14 | C |
| ATOM | 7070 | CB | GLU | B | 276 | 16.318 | −109.252 | −27.509 | 1.00 | 48.16 | C |
| ATOM | 7071 | CG | GLU | B | 276 | 17.585 | −108.518 | −27.919 | 1.00 | 48.40 | C |
| ATOM | 7072 | CD | GLU | B | 276 | 17.459 | −107.006 | −27.902 | 1.00 | 53.03 | C |
| ATOM | 7073 | OE1 | GLU | B | 276 | 16.448 | −106.467 | −27.388 | 1.00 | 53.65 | O |
| ATOM | 7074 | OE2 | GLU | B | 276 | 18.397 | −106.348 | −28.405 | 1.00 | 56.60 | O |
| ATOM | 7075 | C | GLU | B | 276 | 15.344 | −111.449 | −26.770 | 1.00 | 45.44 | C |
| ATOM | 7075 | O | GLU | B | 276 | 15.358 | −111.375 | −25.538 | 1.00 | 43.62 | O |
| ATOM | 7077 | N | ASP | B | 277 | 14.406 | −112.131 | −27.432 | 1.00 | 43.21 | N |
| ATOM | 7078 | CA | ASP | B | 277 | 13.460 | −112.961 | −26.674 | 1.00 | 46.85 | C |
| ATOM | 7079 | CB | ASP | B | 277 | 12.189 | −113.298 | −27.468 | 1.00 | 45.86 | C |
| ATOM | 7080 | CG | ASP | B | 277 | 12.426 | −114.311 | −28.561 | 1.00 | 50.97 | C |
| ATOM | 7081 | OD1 | ASP | B | 277 | 13.553 | −114.870 | −28.659 | 1.00 | 50.06 | O |
| ATOM | 7082 | OD2 | ASP | B | 277 | 11.465 | −114.548 | −29.329 | 1.00 | 50.17 | O |
| ATOM | 7083 | C | ASP | B | 277 | 14.134 | −114.224 | −26.138 | 1.00 | 50.02 | C |
| ATOM | 7084 | O | ASP | B | 277 | 13.469 | −115.091 | −25.568 | 1.00 | 50.76 | O |
| ATOM | 7085 | N | CYS | B | 278 | 15.453 | −114.321 | −26.343 | 1.00 | 50.54 | N |
| ATOM | 7086 | CA | CYS | B | 278 | 16.278 | −115.361 | −25.7361 | 1.00 | 47.79 | C |
| ATOM | 7087 | CB | CYS | B | 278 | 15.925 | −115.523 | −24.254 | 1.00 | 49.58 | C |
| ATOM | 7088 | SG | CYS | B | 278 | 16.583 | −114.211 | −23.206 | 1.00 | 51.58 | S |
| ATOM | 7089 | C | CYS | B | 278 | 16.130 | −116.694 | −26.439 | 1.00 | 48.40 | C |
| ATOM | 7090 | O | CYS | B | 278 | 16.607 | −117.706 | −25.945 | 1.00 | 55.76 | O |
| ATOM | 7091 | N | SER | B | 279 | 15.476 | −116.696 | −27.593 | 1.00 | 46.32 | N |
| ATOM | 7092 | CA | SER | B | 279 | 15.237 | −117.927 | −28.314 | 1.00 | 47.21 | C |
| ATOM | 7093 | CB | SER | B | 279 | 13.727 | −118.142 | −28.540 | 1.00 | 46.97 | C |
| ATOM | 7094 | OG | SER | B | 279 | 13.250 | −117.388 | −29.652 | 1.00 | 45.25 | O |
| ATOM | 7095 | C | SER | B | 279 | 15.982 | −117.991 | −29.650 | 1.00 | 45.71 | C |
| ATOM | 7096 | O | SER | B | 279 | 16.296 | −116.981 | −30.257 | 1.00 | 45.19 | C |
| ATOM | 7097 | N | LEU | B | 280 | 16.247 | −119.210 | −30.095 | 1.00 | 49.75 | N |
| ATOM | 7098 | CA | LEU | B | 280 | 16.672 | −119.481 | −31.458 | 1.00 | 47.47 | C |
| ATOM | 7099 | CB | LEU | B | 280 | 17.897 | −120.423 | −31.466 | 1.00 | 46.54 | C |
| ATOM | 7100 | CG | LEU | B | 280 | 18.150 | −121.337 | −32.683 | 1.00 | 45.50 | C |
| ATOM | 7101 | CD1 | LEU | B | 280 | 18.524 | −120.569 | −33.934 | 1.00 | 41.10 | C |
| ATOM | 7102 | CD2 | LEU | B | 280 | 19.228 | −122.353 | −32.364 | 1.00 | 49.87 | C |
| ATOM | 7103 | C | LEU | B | 280 | 15.507 | −120.108 | −32.213 | 1.00 | 46.68 | C |
| ATOM | 7104 | O | LEU | B | 280 | 14.743 | −120.912 | −31.657 | 1.00 | 51.00 | O |
| ATOM | 7105 | N | ALA | B | 281 | 15.356 | −119.721 | −33.468 | 1.00 | 45.19 | N |
| ATOM | 7106 | CA | ALA | B | 281 | 14.421 | −120.407 | −34.356 | 1.00 | 52.56 | C |
| ATOM | 7107 | CB | ALA | B | 281 | 13.167 | −119.575 | −34.575 | 1.00 | 52.46 | C |
| ATOM | 7108 | C | ALA | B | 281 | 15.089 | −120.740 | −35.686 | 1.00 | 52.88 | C |
| ATOM | 7109 | O | ALA | B | 281 | 16.003 | −120.038 | −36.125 | 1.00 | 56.40 | O |
| ATOM | 7110 | N | VAL | B | 282 | 14.043 | −121.626 | −36.309 | 1.00 | 55.57 | N |
| ATOM | 7111 | CA | VAL | B | 282 | 15.147 | −122.227 | −37.618 | 1.00 | 54.40 | C |
| ATOM | 7112 | CB | VAL | B | 282 | 15.949 | −123.544 | −37.557 | 1.00 | 55.94 | C |
| ATOM | 7113 | CG1 | VAL | B | 282 | 16.279 | −124.038 | −38.962 | 1.00 | 54.62 | C |
| ATOM | 7114 | CG2 | VAL | B | 282 | 17.226 | −123.352 | −36.741 | 1.00 | 53.67 | C |
| ATOM | 7115 | C | VAL | B | 282 | 13.977 | −122.339 | −38.586 | 1.00 | 56.51 | C |
| ATOM | 7116 | O | VAL | B | 282 | 12.979 | −123.032 | −38.314 | 1.00 | 58.40 | O |
| ATOM | 7117 | N | LEU | B | 283 | 14.118 | −121.646 | −39.713 | 1.00 | 54.10 | N |
| ATOM | 7118 | CA | LEU | B | 283 | 13.064 | −121.536 | −40.704 | 1.00 | 58.13 | C |
| ATOM | 7119 | CB | LEU | B | 283 | 12.646 | −120.070 | −40.855 | 1.00 | 54.66 | C |
| ATOM | 7120 | CG | LEU | B | 283 | 12.579 | −119.264 | −39.557 | 1.00 | 51.52 | C |
| ATOM | 7121 | CD1 | LEU | B | 283 | 12.886 | −117.798 | −39.824 | 1.00 | 50.12 | C |
| ATOM | 7122 | CD2 | LEU | B | 283 | 11.239 | −119.458 | −38.864 | 1.00 | 46.58 | C |
| ATOM | 7123 | C | LEU | B | 283 | 13.523 | −122.065 | −42.055 | 1.00 | 63.48 | C |
| ATOM | 7124 | O | LEU | B | 283 | 14.722 | −122.029 | −42.381 | 1.00 | 64.78 | O |
| ATOM | 7125 | N | ASP | B | 284 | 12.560 | −122.542 | −42.839 | 1.00 | 64.15 | N |
| ATOM | 7126 | CA | ASP | B | 284 | 12.823 | −122.975 | −44.211 | 1.00 | 67.77 | C |
| ATOM | 7127 | CB | ASP | B | 284 | 11.848 | −124.078 | −44.631 | 1.00 | 67.86 | C |
| ATOM | 7128 | CG | ASP | B | 284 | 10.391 | −123.664 | −44.482 | 1.00 | 67.69 | C |
| ATOM | 7129 | OD1 | ASP | B | 284 | 10.033 | −122.518 | −44.851 | 1.00 | 69.00 | O |
| ATOM | 7130 | OD2 | ASP | B | 284 | 9.605 | −124.495 | −43.993 | 1.00 | 65.68 | O |
| ATOM | 7131 | C | ASP | B | 284 | 12.747 | −121.806 | −45.193 | 1.00 | 68.67 | C |
| ATOM | 7132 | O | ASP | B | 284 | 12.651 | −120.638 | −44.785 | 1.00 | 65.40 | O |
| ATOM | 7133 | N | SER | B | 285 | 12.767 | −122.143 | −46.485 | 1.00 | 68.90 | N |
| ATOM | 7134 | CA | SER | B | 285 | 12.762 | −121.161 | −47.566 | 1.00 | 69.65 | C |
| ATOM | 7135 | CB | SER | B | 285 | 12.802 | −121.869 | −48.922 | 1.00 | 69.04 | C |
| ATOM | 7136 | OG | SER | B | 285 | 11.640 | −122.657 | −49.109 | 1.00 | 68.13 | O |
| ATOM | 7137 | C | SER | B | 285 | 11.544 | −120.238 | −47.506 | 1.00 | 68.00 | C |
| ATOM | 7138 | O | SER | B | 285 | 11.637 | −119.068 | −47.867 | 1.00 | 65.95 | O |
| ATOM | 7139 | N | SER | B | 286 | 10.415 | −120.773 | −47.048 | 1.00 | 62.57 | N |
| ATOM | 7140 | CA | SER | B | 286 | 9.162 | −120.031 | −47.038 | 1.00 | 63.52 | C |
| ATOM | 7141 | CB | SER | B | 286 | 8.004 | −120.954 | −47.424 | 1.00 | 66.13 | C |
| ATOM | 7142 | OG | SER | B | 286 | 7.696 | −121.347 | −46.361 | 1.00 | 69.98 | O |
| ATOM | 7143 | C | SER | B | 286 | 8.891 | −119.166 | −45.682 | 1.00 | 61.46 | C |
| ATOM | 7144 | O | SER | B | 286 | 7.793 | −118.871 | −45.431 | 1.00 | 57.05 | O |
| ATOM | 7145 | N | LEU | B | 287 | 9.903 | −119.158 | −44.816 | 1.00 | 66.81 | N |
| ATOM | 7146 | CA | LEU | B | 287 | 9.829 | −118.705 | −43.493 | 1.00 | 66.82 | C |
| ATOM | 7147 | CB | LEU | B | 287 | 9.430 | −117.118 | −43.620 | 1.00 | 61.35 | C |
| ATOM | 7148 | CG | LEU | B | 287 | 10.510 | −116.145 | −43.835 | 1.00 | 57.80 | C |
| ATOM | 7149 | CD1 | LEU | B | 287 | 11.741 | −116.616 | −44.596 | 1.00 | 58.11 | C |
| ATOM | 7150 | CD2 | LEU | B | 787 | 9.908 | −114.932 | −44.516 | 1.00 | 58.51 | C |
| ATOM | 7151 | C | LEU | B | 287 | 8.921 | −119.440 | −42.505 | 1.00 | 68.22 | C |
| ATOM | 7152 | O | LEU | B | 287 | 8.461 | −118.863 | −41.516 | 1.00 | 70.14 | O |
| ATOM | 7153 | N | SER | B | 288 | 8.680 | −120.717 | −42.778 | 1.00 | 65.37 | N |
| ATOM | 7154 | CA | SER | B | 288 | 7.903 | −121.558 | −41.886 | 1.00 | 68.93 | C |
| ATOM | 7155 | CB | SER | B | 288 | 7.219 | −122.863 | −42.689 | 1.00 | 67.89 | C |
| ATOM | 7156 | OG | SER | B | 288 | 5.868 | −122.803 | −42.307 | 1.00 | 69.13 | O |
| ATOM | 7157 | C | SER | B | 288 | 8.818 | −122.136 | −40.795 | 1.00 | 68.12 | C |
| ATOM | 7158 | O | SER | B | 288 | 10.012 | −122.166 | −41.027 | 1.00 | 69.43 | O |
| ATOM | 7159 | N | GLU | B | 289 | 8.263 | −122.161 | −39.608 | 1.00 | 66.86 | N |
| ATOM | 7160 | CA | GLU | B | 289 | 9.072 | −122.765 | −38.460 | 1.00 | 69.71 | C |
| ATOM | 7161 | CB | GLU | B | 289 | 8.438 | −122.289 | −37.142 | 1.00 | 70.97 | C |
| ATOM | 7162 | CG | GLU | B | 289 | 9.294 | −122.557 | −35.906 | 1.00 | 73.55 | C |
| ATOM | 7163 | CD | GLU | B | 289 | 8.622 | −122.118 | −34.580 | 1.00 | 74.44 | C |
| ATOM | 7164 | OE1 | GLU | B | 289 | 7.800 | −121.276 | −34.517 | 1.00 | 70.37 | O |
| ATOM | 7165 | OE2 | GLU | B | 289 | 8.944 | −122.190 | −33.574 | 1.00 | 77.32 | O |
| ATOM | 7166 | C | GLU | B | 289 | 9.315 | −124.178 | −38.436 | 1.00 | 69.85 | C |
| ATOM | 7167 | O | GLU | B | 289 | 8.374 | −125.971 | −38.412 | 1.00 | 69.92 | O |
| ATOM | 7168 | N | LEU | B | 290 | 10.588 | −124.861 | −38.442 | 1.00 | 68.55 | N |
| ATOM | 7169 | CA | LEU | B | 290 | 10.993 | −126.063 | −38.331 | 1.00 | 68.20 | C |
| ATOM | 7170 | CB | LEU | B | 290 | 12.135 | −126.367 | −39.308 | 1.00 | 74.77 | C |
| ATOM | 7171 | CG | LEU | B | 290 | 11.866 | −127.149 | −40.595 | 1.00 | 81.50 | C |
| ATOM | 7172 | CD1 | LEU | B | 290 | 11.420 | −126.250 | −41.741 | 1.00 | 79.58 | C |
| ATOM | 7173 | CD2 | LEU | B | 290 | 13.127 | −127.509 | −40.985 | 1.00 | 90.47 | C |
| ATOM | 7174 | C | LEU | B | 290 | 11.459 | −126.415 | −36.919 | 1.00 | 65.96 | C |
| ATOM | 7175 | O | LEU | B | 290 | 11.279 | −127.347 | −36.463 | 1.00 | 61.05 | O |
| ATOM | 7176 | N | PHE | B | 291 | 12.070 | −125.444 | −36.243 | 1.00 | 62.75 | N |
| ATOM | 7177 | CA | PHE | B | 291 | 12.714 | −12.861 | −34.953 | 1.00 | 62.28 | C |
| ATOM | 7178 | CB | PHE | B | 291 | 14.185 | −126.041 | −35.185 | 1.00 | 59.06 | C |
| ATOM | 7179 | CG | PHE | B | 291 | 14.924 | −126.115 | −33.951 | 1.00 | 58.67 | C |
| ATOM | 7180 | CD1 | PHE | B | 291 | 14.458 | −127.191 | −33.193 | 1.00 | 60.75 | C |
| ATOM | 7181 | CE1 | PHE | B | 291 | 15.165 | −128.038 | −32.074 | 1.00 | 62.50 | C |
| ATOM | 7182 | CZ | PHE | B | 291 | 16.360 | −127.422 | −31.712 | 1.00 | 60.46 | C |
| ATOM | 7183 | CE2 | PHE | B | 291 | 16.842 | −126.155 | −32.466 | 1.00 | 58.21 | C |
| ATOM | 7184 | CD2 | PHE | B | 291 | 16.131 | −125.916 | −33.579 | 1.00 | 57.81 | C |
| ATOM | 7185 | C | PHE | B | 291 | 12.609 | −124.390 | −34.107 | 1.00 | 66.10 | C |
| ATOM | 7186 | O | PHE | B | 291 | 12.329 | −123.303 | −34.622 | 1.00 | 67.99 | O |
| ATOM | 7187 | N | ARG | B | 292 | 12.823 | −124.346 | −32.802 | 1.00 | 66.87 | N |
| ATOM | 7188 | CA | ARG | B | 292 | 12.856 | −123.440 | −31.854 | 1.00 | 61.13 | C |
| ATOM | 7189 | CB | ARG | B | 292 | 11.429 | −122.589 | −31.508 | 1.00 | 66.67 | C |
| ATOM | 7190 | CG | ARG | B | 292 | 11.305 | −121.953 | −30.403 | 1.00 | 61.82 | C |
| ATOM | 7191 | CD | ARG | B | 292 | 10.095 | −121.071 | −30.641 | 1.00 | 64.27 | C |
| ATOM | 7192 | NE | ARG | B | 292 | 10.514 | −119.115 | −30.981 | 1.00 | 65.15 | N |
| ATOM | 7193 | CZ | ARG | B | 292 | 10.238 | −119.089 | −32.120 | 1.00 | 57.70 | C |
| ATOM | 7194 | NH1 | ARG | B | 292 | 10.680 | −117.865 | −32.302 | 1.00 | 55.38 | N |
| ATOM | 7195 | NH2 | ARG | B | 292 | 9.514 | −119.668 | −33.063 | 1.00 | 58.53 | N |
| ATOM | 7196 | C | ARG | B | 292 | 13.587 | −123.533 | −30.624 | 1.00 | 60.18 | C |
| ATOM | 7197 | O | ARG | B | 292 | 13.356 | −1265.050 | −30.176 | 1.00 | 61.77 | O |
| ATOM | 7198 | N | SER | B | 293 | 14.486 | −123.112 | −30.089 | 1.00 | 62.62 | N |
| ATOM | 7199 | CA | SER | B | 293 | 15.203 | −123.473 | −28.862 | 1.00 | 57.85 | C |
| ATOM | 7200 | CB | SER | B | 293 | 16.628 | −123.816 | −29.177 | 1.00 | 51.43 | C |
| ATOM | 7201 | OG | SER | B | 293 | 17.311 | −124.252 | −27.983 | 1.00 | 48.58 | O |
| ATOM | 7202 | C | SER | B | 293 | 15.224 | −122.381 | −27.786 | 1.00 | 59.30 | C |
| ATOM | 7203 | O | SER | B | 293 | 15.706 | −121.266 | −28.011 | 1.00 | 61.97 | O |
| ATOM | 7204 | N | GLN | B | 294 | 14.715 | −122.730 | −26.611 | 1.00 | 60.15 | N |
| ATOM | 7205 | CA | GLN | B | 294 | 14.810 | −121.891 | −25.434 | 1.00 | 58.52 | C |
| ATOM | 7206 | CB | GLN | B | 294 | 13.554 | −122.039 | −24.580 | 1.00 | 60.78 | C |
| ATOM | 7207 | CS | GLN | B | 294 | 12.506 | −120.968 | −24.825 | 1.00 | 69.29 | C |
| ATOM | 7208 | CD | GLN | B | 294 | 11.481 | −121.383 | −25.848 | 1.00 | 72.78 | C |
| ATOM | 7209 | OE1 | GLN | B | 294 | 10.830 | −122.418 | −25.705 | 1.00 | 78.23 | O |
| ATOM | 7210 | NE2 | GLN | B | 294 | 11.322 | −120.573 | −26.885 | 1.00 | 74.79 | N |
| ATOM | 7211 | C | GLN | B | 294 | 16.042 | −122.203 | −24.582 | 1.00 | 58.03 | C |
| ATOM | 7212 | O | GLN | B | 294 | 16.152 | −121.724 | −23.453 | 1.00 | 57.21 | O |
| ATOM | 7213 | N | ALA | B | 295 | 16.969 | −122.996 | −25.111 | 1.00 | 55.53 | N |
| ATOM | 7214 | CA | ALA | B | 295 | 18.171 | −123.380 | −24.353 | 1.00 | 58.30 | C |
| ATOM | 7215 | CB | ALA | B | 295 | 19.168 | −124.065 | −25.261 | 1.00 | 55.38 | C |
| ATOM | 7216 | C | ALA | B | 295 | 18.846 | −122.206 | −23.645 | 1.00 | 57.69 | C |
| ATOM | 7217 | O | ALA | B | 295 | 19.459 | −122.361 | −22.575 | 1.00 | 51.39 | O |
| ATOM | 7218 | N | HIS | B | 296 | 18.750 | −121.029 | −24.251 | 1.00 | 59.48 | N |
| ATOM | 7219 | CA | HIS | B | 296 | 19.381 | −119.858 | −23.661 | 1.00 | 60.91 | C |
| ATOM | 7220 | CB | HIS | B | 296 | 19.782 | −118.863 | −24.737 | 1.00 | 55.79 | C |
| ATOM | 7221 | CG | HIS | B | 296 | 21.097 | −119.175 | −25.366 | 1.1.00 | 58.58 | C |
| ATOM | 7222 | ND1 | HIS | B | 296 | 22.290 | −119.079 | −24.681 | 1.00 | 60.13 | N |
| ATOM | 7223 | CE1 | HIS | B | 296 | 23.285 | −119.403 | −25.486 | 1.00 | 56.70 | C |
| ATOM | 7224 | NE2 | HIS | B | 296 | 22.781 | −119.712 | −26.667 | 1.00 | 55.91 | N |
| ATOM | 7225 | CD2 | HIS | B | 296 | 21.412 | −119.586 | −26.616 | 1.00 | 59.19 | C |
| ATOM | 7226 | C | HIS | B | 296 | 18.491 | −119.208 | −22.618 | 1.00 | 60.84 | C |
| ATOM | 7227 | O | HIS | B | 296 | 17.258 | −119.281 | −22.704 | 1.00 | 56.96 | O |
| ATOM | 7228 | N | ARG | B | 297 | 19.123 | −118.591 | −21.624 | 1.00 | 58.38 | N |
| ATOM | 7229 | CA | ARG | B | 297 | 18.376 | −117.865 | −20.604 | 1.00 | 62.80 | C |
| ATOM | 7230 | CB | ARG | B | 297 | 18.431 | −118.598 | −19.257 | 1.00 | 66.50 | C |
| ATOM | 7231 | CG | ARG | B | 297 | 19.834 | −118.784 | −18.719 | 1.00 | 76.98 | C |
| ATOM | 7232 | CD | ARG | B | 297 | 19.923 | −119.918 | −17.711 | 1.00 | 83.38 | C |
| ATOM | 7233 | NE | ARG | B | 297 | 21.326 | −120.139 | −17.369 | 1.00 | 89.38 | N |
| ATOM | 7234 | CZ | ARG | B | 297 | 22.046 | −121.183 | −17.770 | 1.00 | 89.10 | C |
| ATOM | 7235 | NH1 | ARG | B | 297 | 21.492 | −122.139 | −18.508 | 1.00 | 85.46 | N |
| ATOM | 7236 | NH2 | ARG | B | 297 | 23.323 | −121.275 | −17.421 | 1.00 | 85.05 | N |
| ATOM | 7237 | C | ARG | B | 297 | 18.802 | −116.386 | −20.511 | 1.00 | 60.97 | C |
| ATOM | 7238 | O | ARG | B | 297 | 18.538 | −115.705 | −19.519 | 1.00 | 60.68 | O |
| ATOM | 7239 | N | ASP | B | 298 | 19.459 | −115.910 | −21.566 | 1.00 | 53.96 | N |
| ATOM | 7240 | CA | ASP | B | 298 | 19.701 | −114.487 | −21.788 | 1.00 | 49.71 | C |
| ATOM | 7241 | CB | ASP | B | 298 | 20.995 | −114.022 | −21.126 | 1.00 | 49.80 | C |
| ATOM | 7242 | CG | ASP | B | 298 | 20.972 | −112.537 | −20.808 | 1.00 | 50.56 | C |
| ATOM | 7243 | OD1 | ASP | B | 298 | 20.075 | −111.826 | −21.313 | 1.00 | 50.90 | O |
| ATOM | 7244 | OD2 | ASP | B | 298 | 21.845 | −112.075 | −20.051 | 1.00 | 53.54 | O |
| ATOM | 7245 | C | ASP | B | 298 | 19.726 | −114.251 | −23.299 | 1.00 | 46.39 | C |
| ATOM | 7246 | O | ASP | B | 298 | 19.504 | −115.201 | −24.040 | 1.00 | 47.43 | O |
| ATOM | 7247 | N | PHE | B | 299 | 19.971 | −113.016 | −23.764 | 1.00 | 44.74 | N |
| ATOM | 7248 | CA | PHE | B | 299 | 20.023 | −112.738 | −25.219 | 1.00 | 45.19 | C |
| ATOM | 7249 | CB | PHE | B | 299 | 20.516 | −111.323 | −25.550 | 1.00 | 42.44 | C |
| ATOM | 7250 | CG | PHE | B | 299 | 19.675 | −110.201 | −24.980 | 1.00 | 41.78 | C |
| ATOM | 7251 | CD1 | PHE | B | 299 | 18.351 | −110.393 | −24.596 | 1.00 | 42.08 | C |
| ATOM | 7252 | CE1 | PHE | B | 299 | 17.600 | −109.335 | −24.090 | 1.00 | 41.31 | C |
| ATOM | 7253 | CZ | PHE | B | 299 | 18.164 | −108.068 | −23.977 | 1.00 | 39.17 | C |
| ATOM | 7254 | CE2 | PHE | B | 299 | 19.472 | −107.858 | −24.372 | 1.00 | 37.06 | C |
| ATOM | 7255 | CD2 | PHE | B | 299 | 20.212 | −108.911 | −24.883 | 1.00 | 40.49 | C |
| ATOM | 7256 | C | PHE | B | 299 | 20.888 | −113.742 | −25.976 | 1.00 | 45.56 | C |
| ATOM | 7257 | O | PHE | B | 299 | 21.929 | −114.166 | −25.474 | 1.00 | 49.81 | O |
| ATOM | 7258 | N | VAL | B | 300 | 20.423 | −114.132 | −27.164 | 1.00 | 48.82 | N |
| ATOM | 7259 | CA | VAL | B | 300 | 21.184 | −114.963 | −28.111 | 1.00 | 45.02 | C |
| ATOM | 7260 | CB | VAL | B | 300 | 20.279 | −115.920 | −28.916 | 1.00 | 45.94 | C |
| ATOM | 7261 | CG1 | VAL | B | 300 | 21.141 | −116.905 | −29.684 | 1.00 | 47.60 | C |
| ATOM | 7262 | CG2 | VAL | B | 300 | 19.294 | −116.666 | −28.019 | 1.00 | 43.45 | C |
| ATOM | 7263 | C | VAL | B | 300 | 21.859 | −114.033 | −29.110 | 1.00 | 44.62 | C |
| ATOM | 7264 | O | VAL | B | 300 | 21.188 | −113.354 | −29.866 | 1.00 | 44.64 | O |
| ATOM | 7265 | N | ARG | B | 301 | 23.182 | −114.017 | −29.126 | 1.00 | 43.62 | N |
| ATOM | 7266 | CA | ARG | B | 301 | 23.909 | −113.059 | −29.938 | 1.00 | 44.35 | C |
| ATOM | 7267 | CB | ARG | B | 301 | 25.072 | −112.129 | −29.127 | 1.00 | 46.69 | C |
| ATOM | 7268 | CG | ARG | B | 301 | 24.606 | −111.888 | −27.837 | 1.00 | 51.57 | C |
| ATOM | 7269 | CD | ARG | B | 301 | 24.413 | −110.197 | −28.049 | 1.00 | 51.74 | C |
| ATOM | 7270 | NE | ARG | B | 301 | 23.048 | −110.921 | −28.379 | 1.00 | 50.59 | N |
| ATOM | 7271 | CZ | ARG | B | 301 | 22.707 | −108.831 | −28.865 | 1.00 | 50.22 | C |
| ATOM | 7272 | NH1 | ARG | B | 301 | 23.630 | −107.909 | −29.107 | 1.00 | 42.14 | N |
| ATOM | 7273 | NH2 | ARG | B | 301 | 21.432 | −108.370 | −29.113 | 1.00 | 54.50 | N |
| ATOM | 7274 | C | ARG | B | 301 | 24.388 | −113.141 | −31.308 | 1.00 | 46.93 | C |
| ATOM | 7275 | O | ARG | B | 301 | 24.503 | −112.747 | −32.239 | 1.00 | 51.07 | O |
| ATOM | 7276 | N | ASP | B | 302 | 24.684 | −114.829 | −31.445 | 1.00 | 50.52 | N |
| ATOM | 7277 | CA | ASP | B | 302 | 25.162 | −115.334 | −32.729 | 1.00 | 51.37 | C |
| ATOM | 7278 | CB | ASP | B | 302 | 26.686 | −115.231 | −32.814 | 1.00 | 50.82 | C |
| ATOM | 7279 | CG | ASP | B | 302 | 27.170 | −114.147 | −34.205 | 1.00 | 53.02 | C |
| ATOM | 7280 | OD1 | ASP | B | 302 | 26.338 | −114.809 | −35.146 | 1.00 | 52.57 | O |
| ATOM | 7281 | OD2 | ASP | B | 302 | 28.378 | −114.558 | −34.351 | 1.00 | 49.56 | O |
| ATOM | 7282 | C | ASP | B | 302 | 24.702 | −116.753 | −33.035 | 1.00 | 53.50 | C |
| ATOM | 7283 | O | ASP | B | 302 | 24.182 | −117.451 | −32.163 | 1.00 | 51.95 | O |
| ATOM | 7284 | N | ALA | B | 303 | 24.871 | −117.154 | −34.295 | 1.00 | 56.36 | N |
| ATOM | 7285 | CA | ALA | B | 303 | 24.748 | −118.551 | −34.704 | 1.00 | 57.12 | C |
| ATOM | 7286 | CB | ALA | B | 303 | 23.333 | −118.142 | −35.164 | 1.00 | 54.66 | C |
| ATOM | 7287 | C | ALA | B | 303 | 25.748 | −118.182 | −35.820 | 1.00 | 58.75 | C |
| ATOM | 7288 | O | ALA | B | 303 | 25.972 | −118.058 | −36.712 | 1.00 | 63.90 | O |
| ATOM | 7289 | N | THR | B | 304 | 26.363 | −120.068 | −35.752 | 1.00 | 57.93 | N |
| ATOM | 7290 | CA | THR | B | 304 | 27.060 | −120.063 | −36.916 | 1.00 | 52.16 | C |
| ATOM | 7291 | CB | THR | B | 304 | 28.595 | −120.149 | −36.897 | 1.00 | 47.51 | C |
| ATOM | 7292 | OG1 | THR | B | 304 | 29.053 | −120.361 | −35.559 | 1.00 | 49.54 | O |
| ATOM | 7293 | CG2 | THR | B | 304 | 29.044 | −119.399 | −37.768 | 1.00 | 51.09 | C |
| ATOM | 7294 | C | THR | B | 304 | 26.698 | −122.109 | −37.130 | 1.00 | 50.22 | C |
| ATOM | 7295 | O | THR | B | 304 | 26.459 | −122.870 | −36.190 | 1.00 | 47.05 | O |
| ATOM | 7296 | N | TRP | B | 305 | 26.641 | −122.471 | −38.401 | 1.00 | 55.05 | N |
| ATOM | 7297 | CA | TRP | B | 305 | 26.424 | −123.837 | −38.796 | 1.00 | 55.44 | C |
| ATOM | 7298 | CB | TRP | B | 305 | 25.705 | −123.852 | −40.126 | 1.00 | 52.55 | C |
| ATOM | 7299 | CG | TRP | B | 305 | 24.348 | −123.235 | −40.088 | 1.00 | 56.07 | C |
| ATOM | 7300 | CD1 | TRP | B | 305 | 24.047 | −121.922 | −40.265 | 1.00 | 55.38 | C |
| ATOM | 7301 | NE1 | TRP | B | 305 | 22.688 | −121.737 | −40.175 | 1.00 | 53.41 | N |
| ATOM | 7302 | CE2 | TRP | B | 305 | 22.084 | −122.941 | −39.941 | 1.00 | 51.41 | C |
| ATOM | 7303 | CD2 | TRP | B | 305 | 23.097 | −123.912 | −39.883 | 1.00 | 53.19 | C |
| ATOM | 7304 | CE3 | TRP | B | 305 | 22.740 | −125.246 | −39.643 | 1.00 | 55.46 | C |
| ATOM | 7305 | CZ3 | TRP | B | 305 | 21.377 | −125.364 | −39.480 | 1.00 | 53.71 | C |
| ATOM | 7306 | CH2 | TRP | B | 305 | 20.396 | −124.372 | −39.554 | 1.00 | 54.60 | C |
| ATOM | 7307 | CZ2 | TRP | B | 305 | 20.729 | −123.256 | −39.780 | 1.00 | 56.40 | C |
| ATOM | 7308 | C | TRP | B | 305 | 27.778 | −124.329 | −38.922 | 1.00 | 62.46 | C |
| ATOM | 7309 | O | TRP | B | 305 | 28.699 | −124.002 | −39.559 | 1.00 | 59.00 | O |
| ATOM | 7310 | N | SER | B | 306 | 27.913 | −125.695 | −38.296 | 1.00 | 70.55 | N |
| ATOM | 7311 | CA | SER | B | 306 | 29.089 | −126.340 | −38.529 | 1.00 | 80.17 | C |
| ATOM | 7312 | CB | SER | B | 306 | 28.932 | −127.896 | −37.833 | 1.00 | 84.07 | C |
| ATOM | 7313 | OG | SER | B | 306 | 29.965 | −128.793 | −38.214 | 1.00 | 93.77 | O |
| ATOM | 7314 | C | SER | B | 306 | 29.318 | −126.142 | −40.035 | 1.00 | 76.72 | C |
| ATOM | 7315 | O | SER | B | 306 | 28.363 | −127.040 | −40.774 | 1.00 | 74.14 | O |
| ATOM | 7316 | N | PRO | B | 307 | 30.574 | −126.548 | −40.499 | 1.00 | 74.43 | N |
| ATOM | 7317 | CA | PRO | B | 307 | 30.877 | −126.907 | −41.890 | 1.00 | 71.27 | C |
| ATOM | 7318 | CB | PRO | B | 307 | 32.225 | −126.228 | −42.156 | 1.00 | 71.07 | C |
| ATOM | 7319 | CG | PRO | B | 307 | 32.842 | −126.011 | −40.814 | 1.00 | 70.99 | C |
| ATOM | 7320 | CD | PRO | B | 307 | 31.763 | −126.069 | −39.766 | 1.00 | 70.62 | C |
| ATOM | 7321 | C | PRO | B | 307 | 30.975 | −128.430 | −42.030 | 1.00 | 67.09 | C |
| ATOM | 7322 | O | PRO | B | 307 | 30.561 | −128.575 | −43.042 | 1.00 | 66.26 | O |
| ATOM | 7323 | N | LEU | B | 308 | 31.475 | −129.086 | −40.984 | 1.00 | 64.53 | N |
| ATOM | 7324 | CA | LEU | B | 308 | 31.704 | −130.526 | −40.940 | 1.00 | 65.78 | C |
| ATOM | 7325 | CB | LEU | B | 308 | 32.624 | −130.865 | −39.757 | 1.00 | 59.97 | C |
| ATOM | 7326 | CG | LEU | B | 308 | 34.148 | −130.617 | −39.821 | 1.00 | 57.56 | C |
| ATOM | 7327 | CD1 | LEU | B | 308 | 34.592 | −129.470 | −40.725 | 1.00 | 54.63 | C |
| ATOM | 7328 | CD2 | LEU | B | 308 | 34.727 | −130.450 | −38.420 | 1.00 | 53.52 | C |
| ATOM | 7329 | C | LEU | B | 308 | 30.409 | −131.335 | −40.844 | 1.00 | 70.02 | C |
| ATOM | 7330 | O | LEU | B | 308 | 30.240 | −132.322 | −41.559 | 1.00 | 75.78 | O |
| ATOM | 7331 | N | ASN | B | 309 | 29.505 | −130.929 | −39.953 | 1.00 | 70.96 | N |
| ATOM | 7332 | CA | ASN | B | 309 | 28.205 | −131.573 | −39.832 | 1.00 | 71.52 | C |
| ATOM | 7333 | CB | ASN | B | 309 | 28.071 | −132.255 | −38.465 | 1.00 | 71.77 | C |
| ATOM | 7334 | CG | ASN | B | 309 | 26.837 | −133.145 | −38.366 | 1.00 | 74.09 | C |
| ATOM | 7335 | OD1 | ASN | B | 309 | 25.860 | −132.982 | −39.108 | 1.00 | 71.34 | O |
| ATOM | 7336 | ND2 | ASN | B | 309 | 26.877 | −134.094 | −37.438 | 1.00 | 74.86 | N |
| ATOM | 7337 | C | ASN | B | 309 | 27.064 | −130.574 | −40.088 | 1.00 | 75.27 | C |
| ATOM | 7338 | O | ASN | B | 309 | 26.655 | −129.815 | −39.204 | 1.00 | 75.33 | O |
| ATOM | 7339 | N | HIS | B | 310 | 26.549 | −130.591 | −41.309 | 1.00 | 76.43 | N |
| ATOM | 7340 | CA | HIS | B | 310 | 25.577 | −129.594 | −41.763 | 1.00 | 79.78 | C |
| ATOM | 7341 | CB | HIS | B | 310 | 25.359 | −129.724 | −43.281 | 1.00 | 83.64 | C |
| ATOM | 7342 | CG | HIS | B | 310 | 26.454 | −129.113 | −44.104 | 1.00 | 86.38 | C |
| ATOM | 7343 | ND1 | HIS | B | 310 | 26.282 | −178.755 | −45.425 | 1.00 | 89.10 | N |
| ATOM | 7344 | CE1 | HIS | B | 310 | 27.407 | −128.237 | −45.889 | 1.00 | 88.78 | C |
| ATOM | 7345 | NE2 | HIS | B | 310 | 28.298 | −128.234 | −44.914 | 1.00 | 86.75 | N |
| ATOM | 7346 | CD2 | HIS | B | 310 | 27.727 | −128.776 | −43.787 | 1.00 | 85.51 | C |
| ATOM | 7347 | C | HIS | B | 310 | 24.233 | −129.557 | −41.018 | 1.00 | 76.78 | C |
| ATOM | 7348 | O | HIS | B | 310 | 23.388 | −128.720 | −41.318 | 1.00 | 79.52 | O |
| ATOM | 7349 | N | SER | B | 311 | 24.025 | −130.446 | −40.054 | 1.00 | 76.12 | N |
| ATOM | 7350 | CA | SER | B | 311 | 22.791 | −130.401 | −39.264 | 1.00 | 74.23 | C |
| ATOM | 7351 | CB | SER | B | 311 | 22.126 | −131.772 | −39.209 | 1.00 | 76.02 | C |
| ATOM | 7352 | OG | SER | B | 311 | 21.324 | −131.975 | −40.360 | 1.00 | 79.56 | O |
| ATOM | 7353 | C | SER | B | 311 | 23.040 | −129.840 | −37.863 | 1.00 | 74.76 | C |
| ATOM | 7354 | O | SER | B | 311 | 22.098 | −129.611 | −37.087 | 1.00 | 65.64 | O |
| ATOM | 7355 | N | LEU | B | 312 | 24.321 | −129.593 | −37.581 | 1.00 | 71.87 | N |
| ATOM | 7356 | CA | LEU | B | 312 | 24.787 | −129.093 | −36.298 | 1.00 | 73.07 | C |
| ATOM | 7357 | CB | LEU | B | 312 | 26.154 | −129.699 | −35.977 | 1.00 | 74.80 | C |
| ATOM | 7358 | CG | LEU | B | 312 | 26.488 | −130.023 | −34.520 | 1.00 | 83.60 | C |
| ATOM | 7359 | CD1 | LEU | B | 312 | 27.276 | −131.330 | −34.452 | 1.00 | 87.20 | C |
| ATOM | 7360 | CD2 | LEU | B | 312 | 27.223 | −128.879 | −33.821 | 1.00 | 82.53 | C |
| ATOM | 7361 | C | LEU | B | 312 | 24.883 | −127.573 | −36.342 | 1.00 | 71.84 | C |
| ATOM | 7362 | O | LEU | B | 312 | 25.450 | −127.011 | −37.257 | 1.00 | 73.02 | U |
| ATOM | 7363 | N | LEU | B | 313 | 24.332 | −126.920 | −35.317 | 1.00 | 69.74 | N |
| ATOM | 7364 | CA | LEU | B | 313 | 24.266 | −125.445 | −35.243 | 1.00 | 64.39 | C |
| ATOM | 7365 | CB | LEU | B | 313 | 22.854 | −124.963 | −35.569 | 1.00 | 61.46 | C |
| ATOM | 7366 | CG | LEU | B | 313 | 22.557 | −123.479 | −35.376 | 1.00 | 55.13 | C |
| ATOM | 7367 | CD1 | LEU | B | 313 | 23.313 | −122.671 | −36.421 | 1.00 | 51.86 | C |
| ATOM | 7368 | CD2 | LEU | B | 313 | 21.054 | −123.250 | −35.479 | 1.00 | 54.09 | C |
| ATOM | 7369 | C | LEU | B | 313 | 24.661 | −124.899 | −33.878 | 1.00 | 53.33 | C |
| ATOM | 7370 | O | LEU | B | 313 | 24.164 | −125.350 | −32.850 | 1.00 | 59.72 | O |
| ATOM | 7371 | N | THR | B | 314 | 25.536 | −123.906 | −33.868 | 1.00 | 55.10 | N |
| ATOM | 7372 | CA | THR | B | 314 | 26.020 | −123.378 | −32.605 | 1.00 | 57.53 | C |
| ATOM | 7373 | CB | THR | B | 314 | 27.548 | −123.518 | −32.479 | 1.00 | 54.42 | C |
| ATOM | 7374 | OG1 | THR | B | 314 | 27.882 | −124.896 | −32.664 | 1.00 | 63.71 | O |
| ATOM | 7375 | CG2 | THR | B | 314 | 28.016 | −123.091 | −31.087 | 1.00 | 54.24 | C |
| ATOM | 7376 | C | THR | B | 314 | 25.556 | −121.954 | −32.339 | 1.00 | 57.26 | C |
| ATOM | 7377 | O | THR | B | 314 | 25.742 | −121.067 | −33.181 | 1.00 | 64.90 | O |
| ATOM | 7378 | N | THR | B | 315 | 24.941 | −121.757 | −31.169 | 1.00 | 51.87 | N |
| ATOM | 7379 | CA | THR | B | 315 | 24.535 | −120.421 | −30.703 | 1.00 | 47.30 | C |
| ATOM | 7380 | CG | THR | B | 315 | 23.024 | −120.331 | −30.419 | 1.00 | 45.79 | C |
| ATOM | 7381 | OG1 | THR | B | 315 | 22.644 | −121.342 | −29.474 | 1.00 | 43.60 | O |
| ATOM | 7382 | CG2 | THR | B | 315 | 22.217 | −120.477 | −31.705 | 1.00 | 43.54 | C |
| ATOM | 7383 | C | THR | B | 315 | 25.246 | −120.038 | −29.432 | 1.00 | 45.57 | C |
| ATOM | 7384 | O | THR | B | 315 | 25.549 | −120.883 | −28.602 | 1.00 | 41.48 | O |
| ATOM | 7385 | N | VAL | B | 316 | 25.521 | −118.753 | −29.281 | 1.00 | 41.31 | N |
| ATOM | 7386 | CA | VAL | B | 316 | 26.041 | −118.243 | −28.017 | 1.00 | 50.47 | C |
| ATOM | 7387 | CB | VAL | B | 316 | 27.525 | −117.819 | −28.144 | 1.00 | 41.18 | C |
| ATOM | 7388 | CG1 | VAL | B | 316 | 28.335 | −118.974 | −28.714 | 1.00 | 46.57 | C |
| ATOM | 7389 | CG2 | VAL | B | 316 | 27.673 | −116.585 | −29.018 | 1.00 | 46.22 | C |
| ATOM | 7390 | C | VAL | B | 316 | 25.154 | −117.111 | −27.471 | 1.00 | 52.60 | C |
| ATOM | 7391 | O | VAL | B | 316 | 24.422 | −166.467 | −28.226 | 1.00 | 52.74 | O |
| ATOM | 7392 | N | GLY | B | 317 | 25.227 | −116.877 | −26.161 | 1.00 | 55.68 | N |
| ATOM | 7393 | CA | GLY | B | 317 | 24.378 | −115.197 | −25.490 | 1.00 | 55.21 | C |
| ATOM | 7394 | C | GLY | B | 317 | 25.087 | −115.068 | −24.441 | 1.00 | 58.20 | C |
| ATOM | 7395 | O | GLY | B | 317 | 26.287 | −115.212 | −24.224 | 1.00 | 58.56 | O |
| ATOM | 7396 | N | TRP | B | 318 | 24.342 | −114.177 | −23.795 | 1.00 | 59.38 | N |
| ATOM | 7397 | CA | TRP | B | 318 | 24.880 | −113.403 | −22.676 | 1.00 | 51.70 | C |
| ATOM | 7398 | CB | TRP | B | 318 | 24.179 | −112.052 | −22.564 | 1.00 | 54.29 | C |
| ATOM | 7399 | CG | TRP | B | 318 | 24.625 | −111.036 | −23.588 | 1.00 | 54.39 | C |
| ATOM | 7400 | CD1 | TRP | B | 318 | 25.611 | −111.184 | −24.512 | 1.00 | 52.29 | C |
| ATOM | 7401 | NE1 | TRP | B | 318 | 25.742 | −110.042 | −25.260 | 1.00 | 50.57 | N |
| ATOM | 7402 | CE2 | TRP | B | 318 | 24.849 | −109.109 | −24.807 | 1.00 | 52.76 | C |
| ATOM | 7403 | CD2 | TRP | B | 318 | 24.120 | −109.702 | −23.753 | 1.00 | 56.72 | C |
| ATOM | 7404 | CE3 | TRP | B | 318 | 23.123 | −108.954 | −23.116 | 1.00 | 53.70 | C |
| ATOM | 7405 | CZ3 | TRP | B | 318 | 22.894 | −107.660 | −23.551 | 1.00 | 55.24 | C |
| ATOM | 7406 | CH2 | TRP | B | 318 | 23.631 | −107.102 | −24.607 | 1.00 | 50.74 | C |
| ATOM | 7407 | CZ2 | TRP | B | 318 | 24.610 | −107.106 | −25.243 | 1.00 | 47.83 | C |
| ATOM | 7408 | C | TRP | B | 318 | 24.794 | −114.168 | −21.365 | 1.00 | 47.18 | C |
| ATOM | 7409 | O | TRP | B | 318 | 25.199 | −113.670 | −20.329 | 1.00 | 47.16 | O |
| ATOM | 7410 | N | ASP | B | 319 | 24.261 | −115.383 | −21.430 | 1.00 | 49.02 | N |
| ATOM | 7411 | CA | ASP | B | 319 | 24.266 | −116.327 | −20.316 | 1.00 | 57.55 | C |
| ATOM | 7412 | CB | ASP | B | 319 | 23.074 | −117.183 | −20.422 | 1.00 | 46.87 | C |
| ATOM | 7413 | CG | ASP | B | 319 | 22.991 | −117.985 | −21.771 | 1.00 | 46.54 | C |
| ATOM | 7414 | OD1 | ASP | B | 319 | 23.904 | −117.810 | −22.627 | 1.00 | 41.95 | O |
| ATOM | 7415 | OD2 | ASP | B | 319 | 21.985 | −118.707 | −21.977 | 1.00 | 45.97 | O |
| ATOM | 7416 | C | ASP | B | 319 | 25.564 | −117.142 | −20.265 | 1.00 | 54.29 | C |
| ATOM | 7417 | O | ASP | B | 319 | 25.729 | −118.004 | −19.403 | 1.00 | 58.33 | O |
| ATOM | 7418 | N | HIS | B | 320 | 26.474 | −116.872 | −21.197 | 1.00 | 53.30 | N |
| ATOM | 7419 | CA | HIS | B | 320 | 27.747 | −117.578 | −21.277 | 1.00 | 53.88 | C |
| ATOM | 7420 | CB | HIS | B | 320 | 28.484 | −117.469 | −19.948 | 1.00 | 55.71 | C |
| ATOM | 7421 | CG | HIS | B | 320 | 29.970 | −117.161 | −20.081 | 1.00 | 59.17 | C |
| ATOM | 7422 | ND1 | HIS | B | 320 | 30.655 | −116.226 | −20.678 | 1.00 | 57.34 | N |
| ATOM | 7423 | CE1 | HIS | B | 320 | 31.950 | −116.682 | −20.638 | 1.00 | 56.72 | C |
| ATOM | 7424 | NE2 | HIS | B | 320 | 32.128 | −117.846 | −20.038 | 1.00 | 57.96 | N |
| ATOM | 7425 | CD2 | HIS | B | 320 | 30.905 | −118.356 | −19.680 | 1.00 | 52.77 | C |
| ATOM | 7426 | C | HIS | B | 320 | 27.601 | −119.048 | −21.709 | 1.00 | 52.76 | C |
| ATOM | 7427 | C | HIS | B | 320 | 28.526 | −119.840 | −21.572 | 1.00 | 54.40 | O |
| ATOM | 7428 | N | GLN | B | 321 | 26.438 | −119.409 | −22.232 | 1.00 | 52.98 | N |
| ATOM | 7429 | CA | GLN | B | 321 | 26.272 | −120.702 | −22.869 | 1.00 | 53.31 | C |
| ATOM | 7430 | CB | GLN | B | 321 | 24.830 | −121.182 | −22.772 | 1.00 | 56.99 | C |
| ATOM | 7431 | CG | GLN | B | 321 | 24.471 | −121.760 | −21.416 | 1.00 | 63.11 | C |
| ATOM | 7432 | CD | GLN | B | 321 | 23.113 | −122.441 | −21.415 | 1.00 | 68.08 | C |
| ATOM | 7433 | OE1 | GLN | B | 321 | 23.024 | −123.671 | −23.331 | 1.00 | 70.50 | O |
| ATOM | 7434 | NE2 | GLN | B | 321 | 22.042 | −121.645 | −21.525 | 1.00 | 67.55 | N |
| ATOM | 7435 | C | GLN | B | 321 | 26.740 | −120.712 | −24.329 | 1.00 | 54.33 | C |
| ATOM | 7436 | C | GLN | B | 321 | 26.479 | −119.769 | −25.092 | 1.00 | 49.82 | O |
| ATOM | 7437 | N | VAL | B | 322 | 27.468 | −121.780 | −24.682 | 1.00 | 55.30 | N |
| ATOM | 7438 | CA | VAL | B | 322 | 27.786 | −122.137 | −26.064 | 1.00 | 48.79 | C |
| ATOM | 7439 | CB | VAL | B | 332 | 29.289 | −122.460 | −26.233 | 1.00 | 45.29 | C |
| ATOM | 7440 | CG1 | VAL | B | 322 | 29.570 | −123.011 | −27.624 | 1.00 | 43.32 | C |
| ATOM | 7441 | CG2 | VAL | B | 322 | 30.144 | −121.225 | −25.995 | 1.00 | 43.27 | C |
| ATOM | 7442 | C | VAL | B | 322 | 26.966 | −123.391 | −26.332 | 1.00 | 50.58 | C |
| ATOM | 7443 | O | VAL | B | 322 | 27.308 | −124.447 | −25.821 | 1.00 | 50.24 | O |
| ATOM | 7444 | N | VAL | B | 323 | 25.866 | −123.272 | −27.079 | 1.00 | 54.00 | N |
| ATOM | 7445 | CA | VAL | B | 323 | 24.966 | −124.415 | −27.300 | 1.00 | 55.64 | C |
| ATOM | 7446 | CB | VAL | B | 323 | 23.493 | −124.076 | −27.006 | 1.00 | 56.96 | C |
| ATOM | 7447 | CG1 | VAL | B | 323 | 22.593 | −125.289 | −27.222 | 1.00 | 52.42 | C |
| ATOM | 7443 | CG2 | VAL | B | 323 | 23.342 | −123.584 | −25.581 | 1.00 | 58.83 | C |
| ATOM | 7449 | C | VAL | B | 323 | 25.085 | −124.953 | −28.716 | 1.00 | 58.43 | C |
| ATOM | 7459 | O | VAL | B | 323 | 25.115 | −124.187 | −29.679 | 1.00 | 61.02 | O |
| ATOM | 7452 | N | HIS | B | 324 | 25.155 | −126.280 | −28.819 | 1.00 | 62.49 | N |
| ATOM | 7452 | CA | HIS | B | 324 | 25.232 | −126.989 | −30.093 | 1.00 | 59.90 | C |
| ATOM | 7453 | CB | HIS | B | 324 | 26.351 | −128.024 | −30.066 | 1.00 | 57.28 | C |
| ATOM | 7454 | CG | HIS | B | 324 | 27.671 | −127.463 | −29.652 | 1.00 | 58.55 | C |
| ATOM | 7455 | ND1 | HIS | B | 324 | 28.393 | −126.598 | −30.450 | 1.00 | 56.29 | N |
| ATOM | 7456 | CE1 | HIS | B | 324 | 29.518 | −126.273 | −29.832 | 1.00 | 58.20 | C |
| ATOM | 7457 | NE2 | HIS | B | 324 | 29.547 | −126.889 | −28.661 | 1.00 | 60.23 | N |
| ATOM | 7458 | CD2 | HIS | B | 324 | 28.401 | −127.638 | −28.523 | 1.00 | 59.59 | C |
| ATOM | 7459 | C | HIS | B | 324 | 23.907 | −127.676 | −30.370 | 1.00 | 58.90 | C |
| ATOM | 7460 | O | HIS | B | 324 | 23.547 | −128.591 | −29.594 | 1.00 | 58.23 | O |
| ATOM | 7461 | N | HIS | B | 325 | 23.177 | −127.197 | −31.318 | 1.00 | 62.99 | N |
| ATOM | 7462 | CA | HIS | B | 325 | 21.846 | −127.692 | −31.632 | 1.00 | 66.47 | C |
| ATOM | 7463 | CB | HTS | B | 325 | 20.904 | −126.530 | −32.033 | 1.00 | 69.59 | C |
| ATOM | 7464 | CG | HIS | B | 325 | 20.877 | −125.377 | −31.068 | 1.00 | 69.21 | C |
| ATOM | 7465 | ND1 | HIS | B | 325 | 19.865 | −125.198 | −30.146 | 1.00 | 68.23 | N |
| ATOM | 7466 | CE1 | HIS | B | 325 | 20.103 | −124.108 | −29.439 | 1.00 | 63.34 | C |
| ATOM | 7467 | NE2 | HIS | B | 325 | 21.222 | −123.562 | −29.880 | 1.00 | 65.95 | N |
| ATOM | 7468 | CO2 | HIS | B | 325 | 21.725 | −124.334 | −30.900 | 1.00 | 66.11 | C |
| ATOM | 7469 | C | HIS | B | 325 | 21.918 | −128.688 | −32.792 | 1.00 | 67.53 | C |
| ATOM | 7470 | O | HIS | B | 325 | 22.685 | −128.499 | −33.748 | 1.00 | 63.87 | O |
| ATOM | 7471 | N | VAL | B | 326 | 21.094 | −129.728 | −32.715 | 1.00 | 66.86 | N |
| ATOM | 7472 | CA | VAL | B | 326 | 20.930 | −130.664 | −33.822 | 1.00 | 67.27 | C |
| ATOM | 7473 | CB | VAL | B | 326 | 20.944 | −132.137 | −33.347 | 1.00 | 69.38 | C |
| ATOM | 7474 | CG1 | VAL | B | 326 | 22.348 | −132.529 | −32.905 | 1.00 | 59.96 | C |
| ATOM | 7475 | CG2 | VAL | B | 326 | 19.937 | −132.362 | −32.219 | 1.00 | 71.13 | C |
| ATOM | 7476 | C | VAL | B | 326 | 19.641 | −130.344 | −34.554 | 1.00 | 65.52 | C |
| ATOM | 7477 | O | VAL | B | 326 | 18.557 | −130.432 | −33.983 | 1.00 | 72.01 | O |
| ATOM | 7478 | N | VAL | B | 327 | 19.762 | −129.971 | −35.820 | 1.00 | 70.08 | N |
| ATOM | 7479 | CA | VAL | B | 327 | 18.615 | −129.459 | −36.568 | 1.00 | 75.93 | C |
| ATOM | 7480 | CB | VAL | B | 327 | 18.929 | −128.097 | −37.238 | 1.00 | 73.56 | C |
| ATOM | 7481 | CG1 | VAL | B | 327 | 17.692 | −127.523 | −37.916 | 1.00 | 72.87 | C |
| ATOM | 7482 | CG2 | VAL | B | 327 | 19.450 | −127.109 | −36.208 | 1.00 | 73.84 | C |
| ATOM | 7483 | C | VAL | B | 327 | 18.080 | −130.457 | −37.600 | 1.00 | 81.04 | C |
| ATOM | 7484 | O | VAL | B | 327 | 18.810 | −130.871 | −38.508 | 1.00 | 82.14 | O |
| ATOM | 7485 | N | PRO | B | 328 | 16.795 | −130.838 | −37.459 | 1.00 | 85.06 | N |
| ATOM | 7486 | CA | PRO | B | 328 | 16.068 | −131.675 | −38.422 | 1.00 | 88.07 | C |
| ATOM | 7487 | CB | PRO | B | 328 | 14.720 | −131.911 | −37.731 | 1.00 | 88.26 | C |
| ATOM | 7488 | CO | PRO | B | 326 | 14.584 | −130.780 | −56.754 | 1.00 | 87.00 | C |
| ATOM | 7489 | CD | PRO | B | 328 | 15.978 | −130.543 | −36.267 | 1.00 | 83.99 | C |
| ATOM | 7490 | C | PRO | B | 328 | 15.850 | −130.982 | −39.769 | 1.00 | 90.33 | C |
| ATOM | 7491 | O | PRO | B | 328 | 16.771 | −130.909 | −40.587 | 1.00 | 91.20 | O |
| HETATM | 7492 | OXT | SAM | C | 1 | 24.948 | −38.342 | −8.475 | 1.00 | 39.37 | O |
| HETATM | 7493 | C | SAM | C | 1 | 24.982 | −38.422 | −7.205 | 1.00 | 44.52 | C |
| HETATM | 7494 | O | SAM | C | 1 | 25.802 | −39.093 | −6.528 | 1.00 | 42.87 | O |
| HETATM | 7495 | CA | SAM | C | 1 | 23.943 | −37.679 | −6.405 | 1.00 | 46.43 | C |
| HETATM | 7496 | N | SAM | C | 1 | 23.478 | −36.542 | −7.192 | 1.00 | 42.46 | N |
| HETATM | 7497 | CB | SAM | C | 1 | 22.812 | −38.627 | −5.940 | 1.00 | 45.76 | C |
| HETATM | 7498 | CG | SAM | C | 1 | 21.983 | −39.275 | −7.063 | 1.00 | 45.03 | C |
| HETATM | 7499 | SD | SAM | C | 1 | 20.677 | −40.147 | −6.469 | 1.00 | 51.91 | S |
| HETATM | 7500 | CE | SAM | C | 1 | 20.218 | −41.305 | −7.574 | 1.00 | 48.79 | C |
| HETATM | 7501 | C5′ | SAM | C | 1 | 19.350 | −39.169 | −6.224 | 1.00 | 43.20 | C |
| HETATM | 7502 | C4′ | SAM | C | 1 | 19.393 | −38.439 | −4.876 | 1.00 | 38.13 | C |
| HETATM | 7503 | C3′ | SAM | C | 1 | 19.245 | −39.380 | −3.688 | 1.00 | 37.58 | C |
| HETATM | 7504 | O3′ | SAM | C | 1 | 20.289 | −39.130 | −2.729 | 1.00 | 34.68 | O |
| HETATM | 7505 | O2′ | SAM | C | 1 | 17.846 | −39.057 | −3.160 | 1.00 | 33.74 | C |
| HETATM | 7506 | O2′ | SAM | C | 1 | 17.721 | −39.226 | −1.761 | 1.00 | 34.10 | O |
| HETATM | 7507 | C2′ | SAM | c | 1 | 17.640 | −37.610 | −3.524 | 1.00 | 34.87 | C |
| HETATM | 7508 | O4′ | SAM | C | 1 | 18.263 | −37.557 | −4.818 | 1.00 | 37.13 | O |
| HETATM | 7509 | N9 | SAM | C | 1 | 16.224 | −37.195 | −3.708 | 1.00 | 34.32 | N |
| HETATM | 7510 | C4 | SAM | C | 1 | 15.631 | −36.076 | −3.272 | 1.00 | 32.31 | C |
| HETATM | 7511 | C5 | SAM | C | 1 | 14.244 | −36.136 | −3.759 | 1.00 | 31.95 | C |
| HETATM | 7512 | N7 | SAM | C | 1 | 14.129 | −37.256 | −4.463 | 1.00 | 34.39 | N |
| HETATM | 7513 | C8 | SAM | C | 1 | 15.329 | −37.888 | −4.427 | 1.00 | 33.49 | C |
| HETATM | 7514 | N3 | SAM | C | 1 | 15.995 | −35.013 | −2.513 | 1.00 | 32.65 | N |
| HETATM | 7515 | C2 | SAM | C | 1 | 15.100 | −34.043 | −2.242 | 1.00 | 33.83 | C |
| HETATM | 7516 | N1 | SAM | C | 1 | 13.805 | −34.054 | −2.665 | 1.00 | 34.68 | N |
| HETATM | 7517 | C6 | SAM | C | 1 | 13.326 | −35.052 | −3.432 | 1.00 | 32.95 | C |
| HETATM | 7518 | N6 | SAM | C | 1 | 12.046 | −35.076 | −3.885 | 1.00 | 33.43 | N |
| HETATM | 7519 | CAU | EPZ | D | 1 | 22.723 | −44.752 | −1.0.742 | 1.00 | 51.57 | C |
| HETATM | 7520 | CAV | EPZ | D | 1 | 23.120 | −43.372 | −10.237 | 1.00 | 47.74 | C |
| HETATM | 7521 | CAW | EPZ | D | 1 | 23.070 | −43.337 | −8.834 | 1.00 | 51.47 | C |
| HETATM | 7522 | CBC | EPZ | D | 1 | 23.798 | −42.341 | −8.164 | 1.00 | 48.55 | C |
| HETATM | 7523 | CBB | EPZ | D | 1 | 23.771 | −42.282 | −6.772 | 1.00 | 45.51 | C |
| HETATM | 7524 | CBA | EPZ | D | 1 | 23.013 | −43.198 | −6.040 | 1.00 | 44.72 | C |
| HETATM | 7525 | CAZ | EPZ | D | 1 | 22.286 | −44.197 | −6.686 | 1.00 | 46.57 | C |
| HETATM | 7526 | CAX | EPZ | D | 1 | 22.310 | −44.273 | −8.084 | 1.00 | 49.86 | C |
| HETATM | 7527 | CAY | EPZ | D | 1 | 21.563 | −45.292 | −8.682 | 1.00 | 49.39 | C |
| HETATM | 7528 | NAT | EPZ | D | 1 | 21.462 | −45.233 | −10.152 | 1.00 | 51.01 | N |
| HETATM | 7529 | CAS | EPZ | D | 1 | 21.177 | −46.598 | −10.669 | 1.00 | 51.46 | C |
| HETATM | 7530 | CAB | EPZ | D | 1 | 19.672 | −46.866 | −11.023 | 1.00 | 51.32 | C |
| HETATM | 7531 | OAA | EPZ | D | 1 | 18.864 | −46.029 | −10.207 | 1.00 | 48.37 | O |
| HETATM | 7532 | CAC | EPZ | D | 1 | 19.246 | −48.360 | −10.868 | 1.00 | 49.56 | C |
| HETATM | 7533 | NAD | EPZ | D | 1 | 20.418 | −49.234 | −10.581 | 1.00 | 51.11 | N |
| HETATM | 7534 | CAE | EPZ | D | 1 | 21.291 | −49.700 | −11.543 | 1.00 | 54.66 | C |
| HETATM | 7535 | OAF | EPZ | D | 1 | 21.202 | −49.498 | −12.761 | 1.00 | 50.34 | O |
| HETATM | 7536 | CAG | EPZ | D | 1 | 22.466 | −50.553 | −11.034 | 1.00 | 57.14 | C |
| HETATM | 7537 | OAH | EPZ | D | 1 | 22.131 | −51.205 | −9.794 | 1.00 | 60.66 | O |
| HETATM | 7538 | CAI | EPZ | D | 1 | 23.253 | −51.465 | −9.196 | 1.00 | 60.22 | C |
| HETATM | 7539 | CAJ | EPZ | D | 1 | 23.353 | −51.460 | −7.813 | 1.00 | 66.20 | C |
| HETATM | 7540 | NAK | EPZ | D | 1 | 22.376 | −50.825 | −7.122 | 1.00 | 69.49 | N |
| HETATM | 7541 | CAL | EPZ | D | 1 | 22.502 | −50.613 | −5.723 | 1.00 | 69.86 | C |
| HETATM | 7542 | CAM | EPZ | D | 1 | 23.640 | −51.062 | −5.043 | 1.00 | 67.83 | C |
| HETATM | 7543 | CAN | EPZ | D | 1 | 24.643 | −51.719 | −5.760 | 1.00 | 64.77 | C |
| HETATM | 7544 | CAO | EPZ | D | 1 | 24.499 | −51.917 | −7.140 | 1.00 | 65.97 | C |
| HETATM | 7545 | CAP | EPZ | D | 1 | 25.499 | −52.170 | −7.868 | 1.00 | 63.10 | C |
| HETATM | 7346 | CAQ | EPZ | D | 1 | 25.380 | −52.771 | −9.247 | 1.00 | 61.31 | C |
| HETATM | 7547 | CAR | EPZ | D | 1 | 24.252 | −52.117 | −9.922 | 1.00 | 59.51 | C |
| HETATM | 7548 | O3 | GOL | G | 1 | 32.520 | −80.088 | −12.097 | 1.00 | 57.51 | O |
| HETATM | 7549 | C3 | GOL | G | 1 | 31.357 | −79.109 | −12.683 | 1.00 | 55.19 | C |
| HETATM | 7550 | C2 | GOL | G | 1 | 31.384 | −79.902 | −14.145 | 1.00 | 53.68 | C |
| HETATM | 7551 | O2 | GOL | G | 1 | 32.190 | −78.909 | −14.797 | 1.00 | 51.85 | O |
| HETATM | 7552 | C1 | GOL | G | 1 | 29.941 | −79.997 | −14.641 | 1.00 | 49.19 | C |
| HETATM | 7553 | O1 | GOL | G | 1 | 29.576 | −78.199 | −15.490 | 1.00 | 47.87 | O |
| HETATM | 7554 | O3 | GOL | G | 2 | 10.722 | −100.090 | −41.603 | 1.00 | 61.14 | O |
| HETATM | 7555 | C3 | GOL | G | 2 | 10.929 | −99.704 | −42.964 | 1.00 | 60.66 | C |
| HETATM | 7556 | C2 | GOL | G | 2 | 11.663 | −100.167 | −43.596 | 1.00 | 65.75 | C |
| HETATM | 7557 | O2 | GOL | G | 2 | 13.077 | −100.124 | −43.519 | 1.00 | 60.59 | O |
| HETATM | 7558 | C1 | GOL | G | 2 | 11.155 | −101.004 | −45.029 | 1.00 | 66.71 | C |
| HETATM | 7559 | O1 | GOL | G | 2 | 10.019 | −101.183 | −45.004 | 1.00 | 67.89 | O |
| HETATM | 7560 | O | HOH | H | 1 | 16.784 | −35.117 | −12.821 | 1.00 | 26.60 | O |
| HETATM | 7561 | O | HOH | H | 2 | 17.742 | −41.335 | −0.744 | 1.00 | 42.67 | O |
| HETATM | 7562 | O | HOH | H | 3 | 19.835 | −110.178 | −28.740 | 1.00 | 45.13 | O |
| HETATM | 7563 | O | HOH | H | 4 | 20.841 | −88.387 | −51.360 | 1.00 | 38.20 | O |
| HETATM | 7564 | O | HOH | H | 5 | 17.359 | −30.527 | −12.966 | 1.00 | 38.96 | O |
| HETATM | 7565 | O | HOH | H | 6 | 22.920 | −99.424 | −21.217 | 1.00 | 30.84 | O |
| HETATM | 7565 | O | HOH | H | 7 | 35.615 | −99.243 | −13.970 | 1.00 | 43.23 | O |
| HETATM | 7567 | O | HOH | H | 8 | 22.190 | −57.389 | −12.757 | 1.00 | 40.88 | O |
| HETATM | 7568 | O | HOH | H | 9 | 9.057 | −29.800 | −14.394 | 1.00 | 39.76 | O |
| HETATM | 7569 | O | HOH | H | 10 | 23.038 | −67.841 | −28.330 | 1.00 | 52.70 | O |
| HETATM | 7570 | O | HOH | H | 11 | 30.503 | −46.141 | 3.138 | 1.00 | 35.20 | O |
| HETATM | 7571 | O | HOH | H | 12 | 9.315 | −26.602 | −3.239 | 1.00 | 41.70 | O |
| HETATM | 7572 | O | HOH | H | 13 | 14.250 | −98.754 | −46.179 | 1.00 | 45.35 | O |
| HETATM | 7573 | O | HOH | H | 14 | 29.724 | −108.034 | −26.437 | 1.00 | 50.04 | O |
| HETATM | 7574 | O | HOH | H | 15 | 14.682 | −71.012 | −20.059 | 1.00 | 42.57 | O |
| HETATM | 7575 | O | HOH | H | 16 | 38.706 | −103.571 | −28.691 | 1.00 | 43.52 | O |
| HETATM | 7576 | O | HOH | H | 17 | 11.001 | −40.391 | −19.729 | 1.00 | 33.38 | O |
| HETATM | 7577 | O | HOH | H | 18 | 7.321 | −94.376 | −17.224 | 1.00 | 27.67 | O |
| HETATM | 7578 | O | HOH | H | 19 | −1.952 | −43.345 | −17.517 | 1.00 | 34.75 | O |
| HETATM | 7579 | O | HOH | H | 20 | 4.556 | −34.455 | −18.713 | 1.00 | 28.46 | O |
| HETATM | 7580 | O | HOH | H | 21 | 4.088 | −96.955 | −23.287 | 1.00 | 37.22 | O |
| HETATM | 7581 | O | HOH | H | 22 | 12.208 | −51.818 | −33.880 | 1.00 | 30.39 | O |
| HETATM | 7582 | O | HOH | H | 23 | 20.838 | −59.597 | −28.999 | 1.00 | 57.47 | O |
| HETATM | 7583 | O | HOH | H | 24 | 28.635 | −107.628 | −61.389 | 1.00 | 40.13 | O |
| HETATM | 7584 | O | HOH | H | 25 | 9.589 | −19.417 | −5.262 | 1.00 | 42.70 | O |
| HETATM | 7585 | O | HOH | H | 26 | 15.846 | −47.832 | −12.560 | 1.00 | 24.84 | O |
| HETATM | 7586 | O | HOH | H | 27 | 27.592 | −110.742 | −33.963 | 1.00 | 36.13 | O |
| HETATM | 7587 | O | HOH | H | 28 | 4.885 | −63.876 | −5.960 | 1.00 | 33.45 | O |
| HETATM | 7588 | O | HOH | H | 29 | 21.316 | −85.204 | −30.587 | 1.00 | 38.59 | O |
| HETATM | 7589 | O | HOH | H | 30 | 25.374 | −88.050 | −25.419 | 1.00 | 35.49 | O |
| HETATM | 7590 | O | HOH | H | 31 | 23.174 | −112.378 | −38.179 | 1.00 | 44.90 | O |
| HETATM | 7591 | O | HOH | H | 32 | 5.153 | −76.303 | −31.579 | 1.00 | 53.23 | O |
| HETATM | 7592 | O | HOH | H | 33 | 11.330 | −57.144 | −41.083 | 1.00 | 42.53 | O |
| HETATM | 7593 | O | HOH | H | 34 | 5.091 | −33.055 | −8.040 | 1.00 | 38.65 | O |
| HETATM | 7594 | O | HOH | H | 35 | 25.114 | −25.647 | 5.246 | 1.00 | 42.49 | O |
| HETATM | 7595 | O | HOH | H | 36 | 1.961 | −59.487 | −36.635 | 1.00 | 50.24 | O |
| HETATM | 7596 | O | HOH | H | 37 | 28.600 | −109.453 | −15.663 | 1.00 | 41.41 | O |
| HETATM | 7597 | O | HOH | H | 38 | −3.523 | −64.888 | −26.622 | 1.00 | 37.50 | O |
| HETATM | 7598 | O | HOH | H | 39 | 4.909 | −35.169 | −5.292 | 1.00 | 36.18 | O |
| HETATM | 7599 | O | HOH | H | 40 | 14.763 | −118.954 | −23.750 | 1.00 | 45.48 | O |
| HETATM | 7600 | O | HOH | H | 41 | 6.485 | −25.953 | −8.693 | 1.00 | 58.07 | O |
| HETATM | 7601 | O | HOH | H | 42 | 35.439 | −87.406 | −1717 | 1.00 | 45.31 | O |
| HETATM | 7602 | O | HOH | H | 43 | 19.479 | −92.750 | −29.067 | 1.00 | 38.38 | O |
| HETATM | 7603 | O | HOH | H | 44 | 2.421 | −38.513 | −11.813 | 1.00 | 32.86 | O |
| HETATM | 7604 | O | HOH | H | 45 | 23.552 | −116.318 | −43.576 | 1.00 | 38.63 | O |
| HETATM | 7605 | O | HOH | H | 46 | 14.791 | −75.614 | −18.928 | 1.00 | 29.07 | O |
| HETATM | 7606 | O | HOH | H | 47 | −5.233 | −52.672 | −32.401 | 1.00 | 46.54 | O |
| HETATM | 7607 | O | HOH | H | 48 | 11.360 | −48.635 | −11.063 | 1.00 | 33.06 | O |
| HETATM | 7608 | O | HOH | H | 49 | 26.386 | −77.555 | −37.301 | 1.00 | 49.02 | O |
| HETATM | 7609 | O | HOH | H | 50 | 33.895 | −68.746 | −21.311 | 1.00 | 51.92 | O |
| HETATM | 7610 | O | HOH | H | 51 | 1.042 | −43.844 | −29.304 | 1.00 | 40.59 | O |
| HETATM | 7611 | O | HOH | H | 52 | 22.933 | −87.329 | −29.611 | 1.00 | 37.07 | O |
| HETATM | 7612 | O | HOH | H | 53 | 1.554 | −63.358 | −22.604 | 1.00 | 31.97 | O |
| HETATM | 7613 | O | HOH | H | 54 | −2.274 | −53.872 | −31.685 | 1.00 | 41.87 | O |
| HETATM | 7614 | O | HOH | H | 55 | 21.090 | −107.487 | 10.151 | 1.00 | 49.14 | O |
| HETATM | 7615 | O | HOH | H | 56 | 10.423 | −20.793 | −2.866 | 1.00 | 51.05 | O |
| HETATM | 7616 | O | HOH | H | 57 | 23.046 | −96.301 | −56.301 | 1.00 | 44.40 | O |
| HETATM | 7617 | O | HOH | H | 58 | 15.964 | −132.318 | −42.684 | 1.00 | 54.88 | O |
| HETATM | 7618 | O | HOH | H | 59 | 26.453 | −48.445 | 3.602 | 1.00 | 42.29 | O |
| HETATM | 7619 | O | HOH | H | 60 | 12.631 | −89.449 | −27.316 | 1.00 | 30.73 | O |
| HETATM | 7620 | O | HOH | H | 61 | 9.556 | −59.929 | −7.632 | 1.00 | 30.93 | O |
| HETATM | 7621 | O | HOH | H | 62 | 22.130 | −91.640 | −29.076 | 1.00 | 33.54 | O |
| HETATM | 7622 | O | HOH | H | 63 | 49.483 | −98.998 | −41.413 | 1.00 | 45.72 | O |
| HETATM | 7623 | O | HOH | H | 64 | 33.835 | −29.850 | −18.339 | 1.00 | 41.41 | O |
| HETATM | 7624 | O | HOH | H | 65 | 17.499 | −57.505 | −33.304 | 1.00 | 41.36 | O |
| HETATM | 7625 | O | HOH | H | 66 | 13.740 | −98.446 | −13.308 | 1.00 | 31.94 | O |
| HETATM | 7626 | O | HOH | H | 67 | 35.697 | −25.622 | −22.751 | 1.00 | 55.14 | O |
| HETATM | 7627 | O | HOH | H | 68 | 34.404 | −108.405 | −56.891 | 1.00 | 47.35 | O |
| HETATM | 7628 | O | HOH | H | 69 | 14.385 | −110.451 | −23.241 | 1.00 | 50.36 | O |
| HETATM | 7629 | O | HOH | H | 70 | 23.613 | −33.720 | 9.520 | 1.00 | 31.17 | O |
| HETATM | 7630 | O | HOH | H | 71 | 2.381 | −32.464 | −18.615 | 1.00 | 41.66 | O |
| HETATM | 7631 | O | HOH | H | 72 | 16.444 | −91.141 | −33.984 | 1.00 | 44.72 | O |
| HETATM | 7632 | O | HOH | H | 73 | 5.308 | −71.399 | −18.877 | 1.00 | 41.24 | O |
| HETATM | 7633 | O | HOH | H | 74 | 37.136 | −36.893 | −16.136 | 1.00 | 48.27 | O |
| HETATM | 7634 | O | HOH | H | 75 | 34.933 | −79.260 | −13.581 | 1.00 | 51.16 | O |
| HETATM | 7635 | O | HOH | H | 76 | 21.596 | −99.878 | −33.901 | 1.00 | 42.32 | O |
| HETATM | 7636 | O | HOH | H | 77 | 22.905 | −99.744 | −27.618 | 1.00 | 39.33 | O |
| HETATM | 7637 | O | HOH | H | 78 | 18.054 | −63.108 | −9.932 | 1.00 | 31.55 | O |
| HETATM | 7638 | O | HOH | H | 79 | 14.309 | −14.662 | −6.035 | 1.00 | 41.32 | O |
| HETATM | 7639 | O | HOH | H | 80 | −0.650 | −58.855 | −7.740 | 1.00 | 50.06 | O |
| HETATM | 7640 | O | HOH | H | 81 | 0.661 | −35.747 | −13.486 | 1.00 | 45.90 | O |
| HETATM | 7641 | O | HOH | H | 82 | 33.705 | −90.174 | −39.223 | 1.00 | 51.55 | O |
| HETATM | 7642 | O | HOH | H | 83 | 32.989 | −42.748 | −4.139 | 1.00 | 41.04 | O |
| HETATM | 7643 | O | HOH | H | 84 | 38.509 | −30.360 | −13.877 | 1.00 | 53.19 | O |
| HETATM | 7644 | O | HOH | H | 85 | 27.533 | −35.918 | −24.997 | 1.00 | 46.70 | O |
| HETATM | 7645 | O | HOH | H | 86 | 19.478 | −83.192 | −46.474 | 1.00 | 43.36 | O |
| HETATM | 7646 | O | HOH | H | 87 | 18.956 | −28.066 | 6.537 | 1.00 | 44.86 | O |
| HETATM | 7647 | O | HOH | H | 88 | −0.796 | −60.903 | −10.870 | 1.00 | 45.87 | O |
| HETATM | 7648 | O | HOH | H | 89 | 12.439 | −74.919 | −17.717 | 1.00 | 44.20 | O |
| HETATM | 7649 | O | HOH | H | 90 | −0.744 | −64.293 | −24.005 | 1.00 | 35.33 | O |
| HETATM | 7650 | O | HOH | H | 91 | 7.517 | −32.918 | −20.098 | 1.00 | 51.47 | O |
| HETATM | 7651 | O | HOH | H | 92 | 34.425 | −72.078 | −19.482 | 1.00 | 54.74 | O |
| HETATM | 7652 | O | HOH | H | 93 | 24.287 | −111.169 | −42.555 | 1.00 | 47.09 | O |
| HETATM | 7653 | O | HOH | H | 94 | 35.128 | −93.423 | −29.689 | 1.00 | 49.69 | O |
| HETATM | 7654 | O | HOH | H | 95 | 8.511 | −39.094 | 0.456 | 1.00 | 52.43 | O |
| HETATM | 7655 | O | HOH | H | 96 | 13.023 | −40.153 | −12.645 | 1.00 | 35.95 | O |
| HETATM | 7656 | O | HOH | H | 97 | 17.195 | −37.187 | −26.224 | 1.00 | 44.65 | O |
| HETATM | 7657 | O | HOH | H | 98 | 15.566 | −36.949 | −24.395 | 1.00 | 46.58 | O |
| HETATM | 7658 | O | HOH | H | 99 | 6.008 | −23.250 | −17.067 | 1.00 | 60.71 | O |
| HETATM | 7659 | O | HOH | H | 100 | 9.617 | −22.302 | −14.916 | 1.00 | 46.84 | O |
| HETATM | 7660 | O | HOH | H | 101 | 9.604 | −44.019 | −3.621 | 1.00 | 46.74 | O |
| HETATM | 7661 | O | HOH | H | 102 | 31.850 | −37.085 | −2.986 | 1.00 | 55.67 | O |
| HETATM | 7662 | O | HOH | H | 103 | 19.758 | −42.172 | −10.196 | 1.00 | 34.59 | O |
| HETATM | 7663 | O | HOH | H | 104 | 25.613 | −34.121 | −5.639 | 1.00 | 41.13 | O |
| HETATM | 7664 | O | HOH | H | 105 | −5.247 | −55.508 | −32.092 | 1.00 | 48.59 | O |
| HETATM | 7665 | O | HOH | H | 106 | −8.037 | −54.584 | −28.473 | 1.00 | 32.83 | Q |
| HETATM | 7666 | O | HOH | H | 107 | 29.553 | −51.002 | −23.149 | 1.00 | 50.36 | O |
| HETATM | 7667 | O | HOH | H | 108 | 31.118 | −49.039 | −22.091 | 1.00 | 45.35 | O |
| HETATM | 7668 | O | HOH | H | 109 | 17.434 | −67.233 | −28.122 | 1.00 | 49.87 | O |
| HETATM | 7669 | O | HOH | H | 110 | 18.723 | −104.005 | −14.762 | 1.00 | 40.39 | O |
| HETATM | 7670 | O | HOH | H | 111 | 16.565 | −105.73 | −14.308 | 1.00 | 35.54 | O |
| HETATM | 7671 | O | HOH | H | 112 | 32.557 | −93.154 | −2.645 | 1.00 | 45.48 | O |
| HETATM | 7672 | O | HOH | H | 113 | 26.529 | −89.517 | −30.451 | 1.00 | 45.86 | O |
| HETATM | 7673 | O | HOH | H | 114 | 40.988 | −90.58 | −25.626 | 1.00 | 43.01 | O |
| HETATM | 7674 | O | HOH | H | 115 | 47.213 | −71.57 | −32.777 | 1.00 | 52.96 | O |
| HETATM | 7675 | O | HOH | H | 116 | 16.440 | −71.676 | −17.684 | 1.00 | 36.80 | O |
| HETATM | 7676 | O | HOH | H | 117 | −1.199 | −45.264 | −27.906 | 1.00 | 54.47 | O |
| HETATM | 7677 | O | HOH | H | 118 | 14.933 | −60.093 | −7.186 | 1.00 | 45.47 | O |
| HETATM | 7678 | O | HOH | H | 119 | 15.927 | −26.388 | −18.997 | 1.00 | 37.82 | O |
| HETATM | 7679 | O | HOH | H | 120 | 26.369 | −37.067 | −28.308 | 1.00 | 52.94 | O |
| HETATM | 7680 | O | HOH | H | 121 | 6.784 | −33.147 | −16.101 | 1.00 | 53.52 | Q |
| HETATM | 7681 | O | HOH | H | 122 | 15.535 | −47.621 | −10.323 | 1.00 | 46.31 | O |
| HETATM | 7682 | O | HOH | H | 123 | 32.051 | −111.111 | −42.734 | 1.00 | 40.06 | O |
| HETATM | 7683 | O | HOH | H | 124 | 35.961 | −110.552 | −17.534 | 1.00 | 62.41 | O |
| HETATM | 7684 | O | HOH | H | 125 | 32.893 | −37.178 | −9.016 | 1.00 | 49.57 | O |
| HETATM | 7685 | O | HOH | H | 126 | 36.147 | −33.604 | 2.775 | 1.00 | 45.74 | O |
| HETATM | 7686 | O | HOH | H | 127 | 1.635 | −52.607 | −4.981 | 1.00 | 38.75 | O |
| HETATM | 7687 | O | HOH | H | 128 | 13.187 | −78.804 | −9.386 | 1.00 | 44.14 | O |
| HETATM | 7688 | O | HOH | H | 129 | 36.477 | −22.477 | 2.798 | 1.00 | 50.98 | O |
| HETATM | 7689 | O | HOH | H | 130 | 26.203 | −39.200 | −3.621 | 1.00 | 39.60 | O |
| HETATM | 7690 | O | HOH | H | 131 | 31.284 | −35.751 | 4.627 | 1.00 | 31.11 | O |
| HETATM | 7691 | O | HOH | H | 132 | 12.838 | −93.333 | −44.729 | 1.00 | 49.54 | O |
| HETATM | 7692 | O | HOH | H | 133 | 32.760 | −99.308 | −22.724 | 1.00 | 41.49 | O |
| HETATM | 7693 | O | HOH | H | 134 | 33.316 | −104.295 | −22.771 | 1.00 | 36.04 | O |
| HETATM | 7694 | O | HOH | H | 135 | 18.705 | −92.438 | −26.542 | 1.00 | 32.30 | O |
| HETATM | 7695 | O | HOH | H | 136 | 27.095 | −90.254 | −27.427 | 1.00 | 49.28 | O |
| HETATM | 7696 | O | HOH | H | 137 | 5.051 | −108.454 | −30.351 | 1.00 | 48.04 | O |
| HETATM | 7697 | O | HOH | H | 138 | 11.053 | −82.680 | −28.616 | 1.00 | 37.08 | O |
| HETATM | 7698 | O | HOH | H | 139 | 20.941 | −87.426 | −12.382 | 1.00 | 44.41 | O |
| HETATM | 7699 | O | HOH | H | 140 | 25.690 | −112.331 | −15.752 | 1.00 | 35.91 | O |

What is claimed is:

1. A compound that can bind PRMT5 of the formula (I):
Ring AA-M-Ring BB;
wherein
Ring AA is an optionally substituted aryl moiety,
Ring BB is an optionally substituted aryl or heteroaryl moiety, wherein the aryl or heteroaryl moiety is capable of forming a cation-pi interaction with S-adenosyl methionine (SAM),
M is an acyclic linker moiety 3-10 atoms in length, which allows for the planes of Ring AA and Ring BB to be between 75° and 105° relative to each other, and includes a carbonyl group, wherein Ring AA is attached directly to the carbonyl group, or to the alpha-carbon of the carbonyl group,
wherein the compound has a biochemical IC₅₀ for PRMT5 of less than 100 nM.

2. The compound of claim 1, wherein Ring AA is a monocyclic aryl moiety.

3. The compound of claim 1, wherein Ring AA is an optionally substituted, fused bicyclic heteroaryl moiety.

4. The compound of claim 1, wherein Ring AA is an unsubstituted, fused bicyclic heteroaryl moiety.

5. The compound of claim 1, wherein Ring AA is a phenyl moiety fused to a heterocyclic moiety.

6. The compound of claim 1, wherein Ring AA is a phenyl moiety fused to a heteroaryl moiety.

7. The compound of claim 1, wherein Ring AA is a phenyl moiety fused to a 5- or 6-membered heteroaryl moiety.

8. The compound of claim 1, wherein Ring BB is an optionally substituted, bicyclic heteroaryl moiety.

9. The compound of claim 1, wherein Ring BB is an optionally substituted, bicyclic heteroaryl moiety with 1-4 nitrogen atoms.

10. The compound of claim 1, wherein Ring BB is an unsubstituted bicyclic heteroaryl moiety.

11. The compound of claim 1, wherein Ring BB is optionally substituted tetrahydroisoquinoline.

12. The compound of claim 1, wherein Ring BB is unsubstituted tetrahydroisoquinoline.

13. The compound of claim 1, wherein Ring BB is optionally substituted isoindoline.

14. The compound of claim 1, wherein Ring BB is unsubstituted isoindoline.

15. The compound of claim 1, wherein Ring BB is an optionally substituted amino-aryl moiety.

16. The compound of claim 1, wherein Ring BB is optionally substituted benzylamine.

17. The compound of claim 1, wherein Ring BB is unsubstituted benzylamine.

18. The compound of claim 1, wherein M is a linker 4-8 atoms in length.

19. The compound of claim 1, wherein M is a linker 4 atoms in length.

20. The compound of claim 1, wherein M is a linker 5 atoms in length.

21. The compound of claim 1, wherein the atoms of M are selected from the group consisting of C, N, O, and S.

22. The compound of claim 1, wherein the atoms of M are selected from the group consisting of C, N, and O.

23. The compound of claim 1, wherein M comprises an amide moiety.

24. The compound of claim 1, wherein M comprises a hydroxyl moiety.

25. The compound of claim 1, wherein M comprises a sulfonamide moiety.

26. The compound of claim 1, wherein M comprises an ester moiety.

27. The compound of claim 1, wherein M provides a distance between Ring AA and Ring BB ranging from approximately 6 Angstroms to approximately 10 Angstroms.

28. The compound of claim 1, wherein L provides a distance between Ring AA and Ring BB ranging from approximately 8 Angstroms to approximately 9 Angstroms.

29. The compound of claim 1, wherein L allows for the planes of Ring AA and Ring BB to be at an angle ranging from 85° to approximately 95°.

30. A compound of the formula 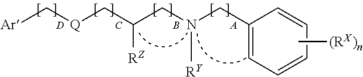
or a pharmaceutically acceptable salt thereof,
wherein
Q is —N(R)C(O)—, —C(O)N(R)—, —N(R)C(O)N(R)—, —N(R)C(O)O—, SO₂NR—, or —OC(O)N(R)—;
each R is independently hydrogen, N-protecting group or optionally substituted C₁₋₆ aliphatic;
Ar′ is a monocyclic or bicyclic aromatic ring having 0-4 heteroatoms independently selected from nitrogen, oxygen, and sulfur, wherein Ar is substituted with 0, 1, 2, 3, 4, or 5 R^{x} groups, as valency permits;
each R^{x} is independently selected from the group consisting of halo, —CN, optionally substituted aliphatic, —OR′, and —N(R″)₂;
R^{y} is hydrogen, amino protecting group or optionally substituted aliphatic;
R^{z} is hydrogen or hydroxyl group;
R′ and R″ are independently hydrogen, N- or O-protecting group, optionally substituted C₁₋₆ aliphatic;
A, B, C, and D are independently 0, 1, or 2;
n is 0, 1, 2, 3, 4, or 5;
the nitrogen substituted by R^{y} optionally forms a ring system with the carbon linked with R^{z}, or forms a ring system with the phenyl group substituted by (R^{x}) n.

31. The compound of claim 30, wherein R^{y} is an optionally substituted aliphatic moiety connected to the ortho position of the phenyl group.

32. The compound of claim 30, wherein R^{z} is a hydroxyl group.

33. The compound of claim 30, wherein Q is —C(O)N(R)—.

34. The compound of claim 30, wherein Ar′ is a monocyclic or bicyclic aromatic ring with one or two nitrogen.

35. The compound of claim 30, wherein Ar′ is

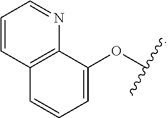 [SMILES: *Oc1cccc2cccnc12]

36. A method for the design and identification of a potential binding compound for protein arginine N-methyltransferase 5 (PRMT5) comprising the steps of:
(a) generating, on a computer, a three-dimensional structure of methyltransferase PRMT5 having the structural coordinates of Table A;
(b) identifying amino acid residues forming an active site in the three-dimensional structure of PRMT5 from step (a), wherein the active site comprises S-adenosyl methionine (SAM) and amino acids Leu319, Phe327, Glu435, Leu437, Glu444, and Phe580, according to Table A;
(c) generating a three-dimensional model of the active site;
(d) designing and/or selecting a compound that potentially binds to the active site using the three-dimensional model of the active site; and
(e) synthesizing and/or choosing the potential binding compound.

37. The method of claim 36, wherein the active site comprises S-adenosyl methionine (SAM) and amino acids Leu319, Phe327, Lys333, Glu435, Leu437, Gly438, Ser439, Glu444, Val503, Ser578, Trp579, and Phe580, according to Table A.

38. The method of claim 36, wherein the active site comprises S-adenosyl methionine (SAM) and amino acids Leu312, Leu319, Thr323, Tyr324, Phe327, Glu328, Lys333, Tyr334, Glu435, Leu437, Gly438, Ser439, Glu444, Val503, Ser578, Trp579, Phe580, and Pro581, according to Table A.

39. A method of identifying a binding compound of protein arginine N-methyltransferase 5 (PRMT5), the method comprising:
computationally identifying a binding compound that binds to PRMT5 using the atomic coordinates of S-adenosyl methionine (SAM) and amino acids Leu319, Phe327, Glu435, Leu437, Glu444, and Phe580, according to Table A.

40. A method of identifying a binding compound of protein arginine N-methyltransferase 5 (PRMT5), the method comprising:
computationally identifying a binding compound that binds to PRMT5 using the atomic coordinates of S-adenosyl methionine (SAM) and amino acids Leu319, Phe327, Lys333, Glu435, Leu437, Gly438, Ser439, Glu444, Val503, Ser578, Trp579, and Phe580, according to Table A.

41. A method of identifying a binding compound of protein arginine N-methyltransferase 5 (PRMT5), the method comprising:
computationally identifying a binding compound that binds to PRMT5 using the atomic coordinates of S-adenosyl methionine (SAM) and amino acids Leu312, Leu319, Thr323, Tyr324, Phe327, Glu328, Lys333, Tyr334, Glu435, Leu437, Gly438, Ser439, Glu444, Val503, Ser578, Trp579, Phe580, and Pro581, according to Table A.

42. A method of identifying a binding compound of protein arginine N-methyltransferase 5 (PRMT5), the method comprising:
a) providing a set of atomic coordinates for a PRMT5 as set forth in Table A; and
b) identifying in silico a binding compound that binds to PRMT5 using the coordinates of step (a).

43. A method of identifying a drug candidate for the treatment of a disease, the method comprising:
a) using the atomic coordinates set forth in Table A to form a three-dimensional structure of PRMT5;
b) selecting a test compound having the best fit with the structure of PRMT5; and
c) assaying the ability of the test compound to modulate PRMT5 activity,
wherein a test compound that modulates PRMT5 activity is considered a drug candidate for treating a disease.

44. A PRMT5 inhibitor having molecular dimensions compatible with the shape of a PRMT5-active site as defined by the atomic coordinates of S-adenosyl methionine (SAM) and amino acids Leu319, Phe327, Glu435, Leu437, Glu444, and Phe580, according to Table A, wherein the compound has a biochemical IC₅₀ for PRMT5 of less than 100 nM.

45. The PRMT5 inhibitor of claim 44, wherein the active site is defined by the atomic coordinates of S-adenosyl methionine (SAM) and amino acids Leu319, Phe327, Lys333, Glu435, Leu437, Gly438, Ser439, Glu444, Val503, Ser578, Trp579, and Phe580, according to Table A.

46. The PRMT5 inhibitor of claim 44, wherein the active site is defined by the atomic coordinates of S-adenosyl methionine (SAM) and amino acids Leu312, Leu319, Thr323, Tyr324, Phe327, Glu328, Lys333, Tyr334, Glu435, Leu437, Gly438, Ser439, Glu444, Val503, Ser578, Trp579, Phe580, and Pro581, according to Table A.

47. The PRMT5 inhibitor of claim 44, wherein the inhibitor is capable of undergoing a pi-cation interaction with SAM.

48. The PRMT5 inhibitor of claim 44, wherein the inhibitor is capable of undergoing a pi-stacking interaction with Phe327.

49. The PRMT5 inhibitor of claim 44, wherein the inhibitor is capable of interacting with Glu444

50. A compound that can bind a PRMT5 of the formula:
Ring AA-M-Ring BB;
wherein
Ring AA is an optionally substituted aromatic moiety;
M is an aliphatic linker;
wherein Ring BB is an aromatic moiety capable of undergoing a pi-cation interaction with S-adenosyl methionine (SAM) and capable of undergoing a pi-stacking interaction with Phe327 of PRMT5;
wherein the planes of Ring AA and Ring BB are at between 75° and 105° relative to each other;
wherein the compound has a biochemical IC₅₀ for PRMT5 of less than 100 nM.

51. A composition comprising a PRMT5 and a compound of the formula:
Ring AA-M-Ring BB;
wherein
Ring AA is an optionally substituted aromatic moiety;
is an aliphatic linker;
wherein Ring BB is an aromatic moiety capable of undergoing a pi-cation interaction with SAM of the PRMT5-SAM complex and capable of undergoing a pi-stacking interaction with Phe327 of PRMT5;
wherein the planes of Ring AA and Ring BB are at between 75° and 105° relative to each other;
wherein the compound has a biochemical IC₅₀ for PRMT5 of less than 100 nM.

52. A computer readable medium comprising the atomic coordinates of PRMT5-Compound A6, as set forth in Table A

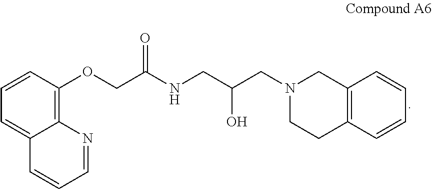 [SMILES: O=C(COc1cccc2cccnc12)NCC(O)CN1CCc2ccccc2C1]

53. The computer readable medium of claim 52 further comprising programming for displaying a molecular model of PRMT5-Compound A6

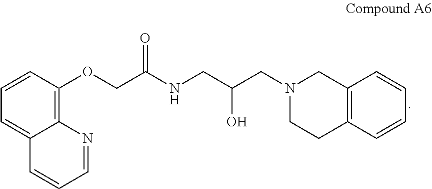 [SMILES: O=C(COc1cccc2cccnc12)NCC(O)CN1CCc2ccccc2C1]

54. The computer readable medium of claim 52 further comprising programming for identifying a binding compound to PRMT5.

55. A crystal structure of PRMT5-Compound A6

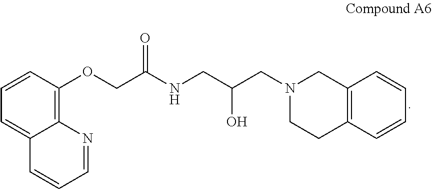 [SMILES: O=C(COc1cccc2cccnc12)NCC(O)CN1CCc2ccccc2C1]

56. A pharmaceutical composition comprising a compound of any one of claims 1-35, 50 or an inhibitor of claims 44-49, or a pharmaceutically acceptable salt thereof, and a pharmaceutically acceptable excipient

57. A kit or packaged pharmaceutical comprising a compound of any one of claims 1-35, 50 or an inhibitor of claims 44-49, or a pharmaceutically acceptable salt thereof, and instructions for use thereof.

58. A method of inhibiting PRMT5 comprising contacting a cell with an effective amount of a compound of any one of claims 1-35, 50 or an inhibitor of claims 44-49, or a pharmaceutically acceptable salt thereof.

59. A method of altering gene expression comprising contacting a cell with an effective amount of a compound of any one of claims 1-35, 50 or an inhibitor of claims 44-49, or a pharmaceutically acceptable salt thereof.

60. A method of altering transcription comprising contacting a cell with an effective amount of a compound of any one of claims 1-35, 50 or an inhibitor of claims 44-49, or a pharmaceutically acceptable salt thereof.

61. The method of any one of claims 58-60, wherein the cell is in vitro.

62. The method of any one of claims 58-60, wherein the cell is in a subject.

63. A method of treating a PRMT5-mediated disorder, comprising administering to a subject in need thereof a therapeutically effective amount of a compound of any one of claims 1-35, 50 or an inhibitor of claims 44-49, or a pharmaceutically acceptable salt thereof, or a pharmaceutical composition of claim 56.

64. The method of claim 63, wherein the disorder is a proliferative disorder.

65. The method of claim 63, wherein the disorder is cancer.

66. The method of claim 65, wherein the cancer is hematopoietic cancer, lung cancer, prostate cancer, melanoma, or pancreatic cancer.

67. The method of claim 63, wherein the disorder is a metabolic disorder.

68. The method of claim 67, wherein the metabolic disorder is diabetes.

69. The method of claim 67, wherein the metabolic disorder is obesity.

70. The method of claim 63, wherein the disorder is a blood disorder.

71. The method of claim 70, wherein the disorder is sickle cell anemia.

72. The method of claim 70, wherein the disorder is β-thalessemia.


SEQUENCE LISTING (7 sequences)

SEQ ID NO: 1 - protein, 637 aa, Homo sapiens
MAAMAVGGAGGSRVSSGRDLNCVPEIADTLGAVAKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

SEQ ID NO: 2 - protein, 620 aa, Homo sapiens
MRGPNSGTEKGRLVIPEKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

SEQ ID NO: 3 - protein, 16 aa, Artificial Sequence
  Synthetic Polypeptide
SGRGKGGKGLGKGGAK

SEQ ID NO: 4 - protein, 9 aa, Artificial Sequence
  Synthetic Polypeptide
MDYKDDDDK

SEQ ID NO: 5 - protein, 7 aa, Artificial Sequence
  Synthetic Polypeptide
MHHHHHH

SEQ ID NO: 6 - protein, 645 aa, Artificial Sequence
  Synthetic Polypeptide
MDYKDDDDKAAMAVGGAGGSRVSSGRDLNCVPEIADTLGAVAKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

SEQ ID NO: 7 - protein, 348 aa, Artificial Sequence
  Synthetic Polypeptide
MHHHHHHRKETPPPLVPPAAREWNLPPNAPACMERQLEAARYRSDGALLLGASSLSGRCWAGSLWLFKDPCAAPNEGFCSAGVQTEAGVADLTWVGERGILVASDSGAVELWELDENETLIVSKFCKYEHDDIVSTVSVLSSGTQAVSGSKDICIKVWDLAQQVVLSSYRAHAAQVTCVAASPHKDSVFLSCSEDNRILLWDTRCPKPASQIGCSAPGYLPTSLAWHPQQSEVFVFGDENGTVSLVDTKSTSCVLSSAVHSQCVTGLVFSPHSVPFLASLSEDCSLAVLDSSLSELFRSQAHRDFVRDATWSPLNHSLLTTVGWDHQVVHHVVPTEPLPAPGPASVTE